(19) United States
(12) Patent Application Publication
(10) Pub. No.: US 2020/0038513 A1
(43) Pub. Date: Feb. 6, 2020

(54) MODULATORS OF FAK PROTEOLYSIS AND ASSOCIATED METHODS OF USE

(71) Applicants: ARVINAS OPERATIONS, INC., New Haven, CT (US); YALE UNIVERSITY, New Haven, CT (US)

(72) Inventors: Craig M. Crews, New Haven, CT (US); Philipp M. Cromm, New Haven, CT (US); Andrew P. Crew, Guilford, CT (US)

(21) Appl. No.: 16/523,219

(22) Filed: Jul. 26, 2019

Related U.S. Application Data
(60) Provisional application No. 62/703,800, filed on Jul. 26, 2018.

(51) Int. Cl. A61K 47/54; A61K 45/06; A61K 31/505; A61K 31/427; A61K 31/454

(52) U.S. Cl. CPC A61K 47/54; A61K 45/06; A61K 31/454; A61K 31/427; A61K 31/505

(57) ABSTRACT

The present disclosure relates to bifunctional compounds, which find utility as modulators of focal adhesion kinase (FAK) or protein tyrosine kinase 2 (PTK2). In particular, the present disclosure is directed to bifunctional compounds, which contain on one end a Von Hippel-Lindau, cereblon, Inhibitors of Apotosis Proteins or mouse double-minute homolog 2 ligand which binds to the respective E3 ubiquitin ligase and on the other end a moiety which binds the target protein, such that the target protein is placed in proximity to the ubiquitin ligase to effect degradation (and inhibition) of target protein. The present disclosure exhibits a broad range of pharmacological activities associated with degradation/inhibition of target protein. Diseases or disorders that result from aggregation or accumulation of the target protein are treated or prevented with compounds and compositions of the present disclosure.

### CROSS-REFERENCE TO RELATED APPLICATIONS

The present application claims the benefit and priority to U.S. Provisional Patent Application No. 62/703,800, filed 26 Jul. 2018 and titled MODULATORS OF PROTEOLYSIS AND ASSOCIATED METHODS OF USE, which is incorporated herein by reference in its entirety for all purposes.

### INCORPORATION BY REFERENCE

U.S. patent application Ser. No. 15/230,354, filed on Aug. 5, 2016, published as U.S. Patent Application Publication No. 2017/0065719; and U.S. patent application Ser. No. 15/206,497 filed 11 Jul. 2016, published as U.S. Patent Application Publication No. 2017/0008904; and U.S. patent application Ser. No. 15/209,648 filed 13 Jul. 2016, published as U.S. Patent Application Publication No. 2017/0037004; and U.S. patent application Ser. No. 15/730,728, filed on Oct. 11, 2017, published as U.S. Patent Application Publication No. 2018/0099940; and U.S. patent application Ser. No. 14/686,640, filed on Apr. 14, 2015, published as U.S. Patent Application Publication No. 2015/0291562; and U.S. patent application Ser. No. 14/792,414, filed on Jul. 6, 2015, published as U.S. Patent Application Publication No. 2016/0058872; and U.S. patent application Ser. No. 14/371,956, filed on Jul. 11, 2014, published as U.S. Patent Application Publication No. 2014/0356322; and U.S. patent application Ser. No. 15/074,820, filed on Mar. 18, 2016, published as U.S. Patent Application Publication No. 2016/0272639; and U.S. patent application Ser. No. 15/885,671, filed Jan. 31, 2018, published as U.S. Patent Application Publication No. 2018/0215731 A1; and International Patent Application No. PCT/US2016/023258, filed Mar. 18, 2016, published as International Patent Application Publication No. WO2016/149668, are incorporated herein by reference in their entirety. Furthermore, all references cited herein are incorporated by reference herein in their entirety.

### FIELD OF THE INVENTION

The description provides bifunctional compounds comprising a target protein binding moiety and a E3 ubiquitin ligase binding moiety, and associated methods of use. The bifunctional compounds are useful as modulators of targeted ubiquitination, especially with respect to a protein tyrosine kinase 2 (PTK2) or Focal Adhesion Kinase (FAK) and/or mutant FAKs, including gain-of-function FAK mutant(s) which are degraded and/or otherwise inhibited by bifunctional compounds according to the present disclosure.

### BACKGROUND

Most small molecule drugs bind enzymes or receptors in tight and well-defined pockets. On the other hand, protein-protein interactions are notoriously difficult to target using small molecules due to their large contact surfaces and the shallow grooves or flat interfaces involved. E3 ubiquitin ligases (of which hundreds are known in humans) confer substrate specificity for ubiquitination, and therefore, are more attractive therapeutic targets than general proteasome inhibitors due to their specificity for certain protein substrates. The development of ligands of E3 ligases has proven challenging, in part due to the fact that they must disrupt protein-protein interactions. However, recent developments have provided specific ligands which bind to these ligases. For example, since the discovery of nutlins, the first small molecule E3 ligase inhibitors, additional compounds have been reported that target E3 ligases but the field remains underdeveloped. For example, since the discovery of Nutlins, the first small molecule E3 ligase mouse double minute 2 homolog (MDM2) inhibitors, additional compounds have been reported that target MDM2 (i.e., human double minute 2 or HDM2) E3 ligases (J. Di, et al. Current Cancer Drug Targets (2011), 11(8), 987-994).

Tumor suppressor gene p53 plays an important role in cell growth arrest and apoptosis in response to DNA damage or stress (A. Vazquez, et al. Nat. Rev. Drug. Dis. (2008), 7, 979-982), and inactivation of p53 has been suggested as one of the major pathway for tumor cell survival (A. J. Levine, et al. Nature (2000), 408, 307-310). In cancer patients, about 50% were found with p53 mutation (M. Hollstein, et al. Science (1991), 233, 49-53), while patients with wild type p53 were often found p53 down regulation by MDM2 through the protein-protein interaction of p53 and MDM2 (P. Chene, et al. Nat. Rev. Cancer (2003), 3, 102-109). Several mechanisms can explain p53 down regulation by MDM2. First, MDM2 binds to N-terminal domain of p53 and blocks expression of p53-responsive genes (J. Momand, et al. Cell (1992), 69, 1237-1245). Second, MDM2 shuttles p53 from nucleus to cytoplasm to facilitate proteolytic degradation (J. Roth, et al. EMBO J. (1998), 17, 554-564). Lastly, MDM2 carries intrinsic E3 ligase activity of conjugating ubiquitin to p53 for degradation through ubiquitin-dependent 26s proteasome system (UPS) (Y. Haupt, et al. Nature (1997) 387, 296-299). As such, because MDM2 functions as E3 ligase, recruiting MDM2 to a disease causing protein and effectuating its ubiquitination and degradation is an approach of high interest for drug discovery.

One E3 ligase with exciting therapeutic potential is the von Hippel-Lindau (VHL) tumor suppressor, the substrate recognition subunit of the E3 ligase complex VCB, which also consists of elongins B and C, Cul2 and Rbx 1. The primary substrate of VHL is Hypoxia Inducible Factor 1α (HIF-1α), a transcription factor that upregulates genes such as the pro-angiogenic growth factor VEGF and the red blood cell inducing cytokine erythropoietin in response to low oxygen levels. The first small molecule ligands of Von Hippel Lindau (VHL) to the substrate recognition subunit of the E3 ligase were generated, and crystal structures were obtained confirming that the compound mimics the binding mode of the transcription factor HIF-1α, the major substrate of VHL.

Cereblon is a protein that in humans is encoded by the CRBN gene. CRBN orthologs are highly conserved from plants to humans, which underscores its physiological importance. Cereblon forms an E3 ubiquitin ligase complex with damaged DNA binding protein 1 (DDB1), Cullin-4A (CUL4A), and regulator of cullins 1 (ROC1). This complex ubiquitinates a number of other proteins. Through a mechanism which has not been completely elucidated, cereblon ubquitination of target proteins results in increased levels of fibroblast growth factor 8 (FGF8) and fibroblast growth factor 10 (FGF10). FGF8 in turn regulates a number of developmental processes, such as limb and auditory vesicle formation. The net result is that this ubiquitin ligase complex is important for limb outgrowth in embryos. In the absence of cereblon, DDB1 forms a complex with DDB2 that functions as a DNA damage-binding protein.

Inhibitors of Apotosis Proteins (IAPs) are a protein family involved in suppressing apoptosis, i.e. cell death. The human IAP family includes 8 members, and numerous other organisms contain IAP homologs. IAPs contain an E3 ligase specific domain and baculoviral IAP repeat (BIR) domains that recognize substrates, and promote their ubiquitination. IAPs promote ubiquitination and can directly bind and inhibit caspases. Caspases are proteases (e.g. caspase-3, caspase-7 and caspace-9) that implement apoptosis. As such, through the binding of caspases, IAPs inhibit cell death. However, pro-apoptotic stimuli can result in the release of mitochondrial proteins DIABLO (also known as second mitrochondria-derived activator of caspases or SMAC) and HTRA2 (also known as Omi). Binding of DIABLO and HTRA2 appears to block IAP activity.

SMAC interacts with essentially all known IAPs including MAP, c-IAP1. c-IAP2. NIL-LAP, Bruce. and survivin. The first four amino acids (AVPI) of mature SMAC bind to a portion of IAPs, which is believed to be essential for blocking the anti-apoptotic effects of IAN.

Bifunctional compounds such as those that are described in U.S. Patent Application Publications 2015-0291562 and 2014-0356322 (incorporated herein by reference), function to recruit endogenous proteins to an E3 ubiquiuin ligase for degradation. In particular, the publications describe bifunctional or proteolysis targeting chimeric (PROTAC) compounds, which find utility as modulators of targeted ubiquitination of a variety of polypeptides and other proteins, which are then degraded and/or otherwise inhibited by the bifunctional compounds.

Focal adhesion kinase (FAK/PTK2) represents a promising cancer target as it is involved in tumor growth, invasion and metastasis. (Cance, W. G., Kurenova, E., Marlowe, T. & Golubovskaya, V. Disrupting the scaffold to improve focal adhesion kinase-targeted cancer therapeutics. Sci. Signal 6, pe10 (2013)). Thus far, FAK targeting has exclusively focused on FAK kinase activity despite strong evidence that FAK exerts a scaffolding role via which Fak is mediating additional signaling cascades (Lee, B. Y., Timpson, P., Horvath, L. G. & Daly, R. J. FAK signaling in human cancer as a target for therapeutics. Pharmacol. Ther. 146, 132-149 (2015)). This additional scaffolding function renders FAK a prime target for small molecule induced protein degradation. FAK degradation might prove especially useful to impede tumor cell mobility and metastasis (Mitra, S. K., Hanson, D. A. & Schlaepfer, D. D. Focal adhesion kinase: in command and control of cell motility. Nat. Rev. Mol. Cell Biol., 6, 56-68 (2005)).

An ongoing need exists in the art for effective treatments for disease associated with overexpression or aggregation of FAK/PTK2. However, non-specific effects, and the inability to target and modulate FAK/PTK2, remain as obstacles to the development of effective treatments. As such, small-molecule therapeutic agents that target FAK/PTK2 and that leverage or potentiate VHL's, cereblon's, MDM2's, and IAPs' substrate specificity would be very useful.

### SUMMARY

The present disclosure describes bifunctional compounds which function to recruit endogenous proteins to an E3 ubiquitin ligase for degradation, and methods of using the same. In particular, the present disclosure provides bifunctional or proteolysis targeting chimeric compounds, which find utility as modulators of targeted ubiquitination of a variety of polypeptides and other proteins, which are then degraded and/or otherwise inhibited by the bifunctional compounds as described herein. An advantage of the compounds provided herein is that a broad range of pharmacological activities is possible, consistent with the degradation/inhibition of targeted polypeptides from virtually any protein class or family. In addition, the description provides methods of using an effective amount of the compounds as described herein for the treatment or amelioration of a disease condition, such as cancer, e.g., solid tumors, carcinoma, adenocarcinoma, cystadenocarcinoma, endometriod carcinoma, mesothelioma, sarcoma, breast cancer, ovarian cancer, lung cancer, head and neck cancer, colorectal cancer, bladder cancer, uterine cancer, prostate cancer, squamous cell carcinoma, leukemia, glioblastoma and renal cancer.

As such, in one aspect the disclosure provides bifunctional compounds, which comprise an E3 ubiquitin ligase binding moiety (i.e., a ligand for an E3 ubquitin ligase or “ULM” group), and a moiety that binds a target protein (i.e., a protein/polypeptide targeting ligand or “PTM” group) such that the target protein/polypeptide is placed in proximity to the ubiquitin ligase to effect degradation (and inhibition) of that protein. In a preferred embodiment, the ULM (ubiquitination ligase modulator) can be Von Hippel-Lindau E3 ubiquitin ligase (VHL) binding moiety (VLM), or a cereblon E3 ubiquitin ligase binding moiety (CLM), or a mouse double minute 2 homolog (MDM2) E3 ubiquitin ligase binding moiety (MLM), or an IAP E3 ubiquitin ligase binding moiety (i.e., a “ILM”). For example, the structure of the bifunctional compound can be depicted as:

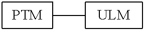

The respective positions of the PTM and ULM moieties (e.g., VLM, CLM, MLM or ILM) as well as their number as illustrated herein is provided by way of example only and is not intended to limit the compounds in any way. As would be understood by the skilled artisan, the bifunctional compounds as described herein can be synthesized such that the number and position of the respective functional moieties can be varied as desired.

In certain embodiments, the bifunctional compound further comprises a chemical linker (“L”). In this example, the structure of the bifunctional compound can be depicted as:

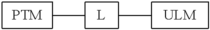

where PTM is a protein/polypeptide targeting moiety, L is a linker, e.g., a bond or a chemical group coupling PTM to ULM, and ULM is a IAP E3 ubiquitin ligase binding moiety, or a Von Hippel-Lindau E3 ubiquitin ligase (VHL) binding moiety (VLM), or a cereblon E3 ubiquitin ligase binding moiety (CLM), or a mouse double minute 2 homolog (MDM2) E3 ubiquitin ligase binding moiety (MLM).

For example, the structure of the bifunctional compound can be depicted as:

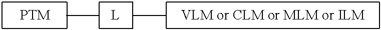

wherein: PTM is a protein/polypeptide targeting moiety; “L” is a linker (e.g. a bond or a chemical linker group) coupling the PTM and at least one of VLM, CLM, MLM, ILM, or a combination thereof; VLM is Von Hippel-Lindau E3 ubiquitin ligase binding moiety that binds to VHL E3 ligase; CLM is cereblon E3 ubiquitin ligase binding moiety that binds to cereblon; MLM is an MDM2 E3 ubiquitin ligase binding moiety that binds MDM2; and ILM is a IAP binding moiety that binds to IAP.

In certain preferred embodiments, the ILM is an AVPI tetrapeptide fragment. As such, in certain additional embodiments, the ILM of the bifunctional compound comprises the amino acids alanine (A), valine (V), proline (P), and isoleucine (I) or their unnatural mimetics, respectively. In additional embodiments, the amino acids of the AVPI tetrapeptide fragment are connected to each other through amide bonds (i.e., —C(O)NH— or —NHC(O)—).

In certain embodiments, the compounds as described herein comprise multiple independently selected ULMs, multiple PTMs, multiple chemical linkers or a combination thereof.

In certain embodiments, ILM comprises chemical moieties such as those described herein.

In additional embodiments, VLM can be hydroxyproline or a derivative thereof. Furthermore, other contemplated VLMs are included in U.S. Patent Application Publication No. 2014/03022523, which as discussed above, is incorporated herein in its entirety.

In an embodiment, the CLM comprises a chemical group derived from an imide, a thioimide, an amide, or a thioamide. In a particular embodiment, the chemical group is a phthalimido group, or an analog or derivative thereof. In a certain embodiment, the CLM is thalidomide, lenalidomide, pomalidomide, analogs thereof, isosteres thereof, or derivatives thereof. Other contemplated CLMs are described in U.S. Patent Application Publication No. 2015/0291562, which is incorporated herein in its entirety.

In certain embodiments, MLM can be nutlin or a derivative thereof. Furthermore, other contemplated MLMs are included in U.S. patent application Ser. No. 15/206,497 filed 11 Jul. 2016, which as discussed above, is incorporated herein in its entirety. In certain additional embodiments, the MLM of the bifunctional compound comprises chemical moieties such as substituted imidazolines, substituted spiro-indolinones, substituted pyrrolidines, substituted piperidinones, substituted morpholinones, substituted pyrrolopyrimidines, substituted imidazolopyridines, substituted thiazoloimidazoline, substituted pyrrolopyrrolidinones, and substituted isoquinolinones.

In additional embodiments, the MLM comprises the core structures mentioned above with adjacent bis-aryl substitutions positioned as cis- or trans-configurations.

In certain embodiments, “L” is a bond. In additional embodiments, the linker “L” is a connector with a linear non-hydrogen atom number in the range of 1 to 20. The connector “L” can contain, but not limited to the functional groups such as ether, amide, alkane, alkene, alkyne, ketone, hydroxyl, carboxylic acid, thioether, sulfoxide, and sulfone. The linker can contain aromatic, heteroaromatic, cyclic, bicyclic and tricyclic moieties. Substitution with halogen, such as Cl, F, Br and I can be included in the linker. In the case of fluorine substitution, single or multiple fluorines can be included.

In certain embodiments, VLM is a derivative of trans-3-hydroxyproline, where both nitrogen and carboxylic acid in trans-3-hydroxyproline are functionalized as amides.

In certain embodiments, CLM is a derivative of piperidine-2,6-dione, where piperidine-2,6-dione can be substituted at the 3-position, and the 3-substitution can be bicyclic hetero-aromatics with the linkage as C—N bond or C—C bond. Examples of CLM can be, but not limited to, pomalidomide, lenalidomide and thalidomide and their derivatives.

In an additional aspect, the description provides therapeutic compositions comprising an effective amount of a compound as described herein or salt form thereof, and a pharmaceutically acceptable carrier. The therapeutic compositions modulate protein degradation and/or inhibition in a patient or subject, for example, an animal such as a human, and can be used for treating or ameliorating disease states or conditions which are modulated through the degraded/inhibited protein. In certain embodiments, the therapeutic compositions as described herein may be used to effectuate the degradation of proteins of interest for the treatment or amelioration of a disease, such a, cancer, (such as, solid tumors, carcinoma, adenocarcinoma, cystadenocarcinoma, endometriod carcinoma, mesothelioma, sarcoma, breast cancer, ovarian cancer, lung cancer, head and neck cancer, colorectal cancer, bladder cancer, uterine cancer, prostate cancer, squamous cell carcinoma, leukemia, glioblastoma and renal cancer.) In yet another aspect, the present disclosure provides a method of ubiquitinating/degrading a target protein in a cell. In certain embodiments, the method comprises administering a bifunctional compound as described herein comprising an ILM and a PTM, a PTM and a VLM, or a PTM and a CLM, or a PTM and a MLM, preferably linked through a linker moiety, as otherwise described herein, wherein the VLM/ILM/CLM/MLM is coupled to the PTM through a linker to target protein that binds to PTM for degradation. Similarly, the PTM can be coupled to VLM or CLM or MLM or ILM through a linker to target a protein or polypeptide for degradation. Degradation of the target protein will occur when the target protein is placed in proximity to the E3 ubiquitin ligase, thus resulting in degradation/inhibition of the effects of the target protein and the control of protein levels. The control of protein levels afforded by the present disclosure provides treatment of a disease state or condition, which is modulated through the target protein by lowering the level of that protein in the cells of a patient.

In still another aspect, the description provides methods for treating or ameliorating a disease, disorder or symptom thereof in a subject or a patient, e.g., an animal such as a human, comprising administering to a subject in need thereof a composition comprising an effective amount, e.g., a therapeutically effective amount, of a compound as described herein or salt form thereof, and a pharmaceutically acceptable carrier, wherein the composition is effective for treating or ameliorating the disease or disorder or symptom thereof in the subject.

In another aspect, the description provides methods for identifying the effects of the degradation of proteins of interest in a biological system using compounds according to the present disclosure.

The preceding general areas of utility are given by way of example only and are not intended to be limiting on the scope of the present disclosure and appended claims. Additional objects and advantages associated with the compositions, methods, and processes of the present disclosure will be appreciated by one of ordinary skill in the art in light of the instant claims, description, and examples. For example, the various aspects and embodiments of the disclosure may be utilized in numerous combinations, all of which are expressly contemplated by the present description. These additional aspects and embodiments are expressly included within the scope of the present disclosure. The publications and other materials used herein to illuminate the background of the disclosure, and in particular cases, to provide additional details respecting the practice, are incorporated by reference.

### BRIEF DESCRIPTION OF THE DRAWINGS

The accompanying drawings, which are incorporated into and form a part of the specification, illustrate several embodiments of the present disclosure and, together with the description, serve to explain the principles of the disclosure. The drawings are only for the purpose of illustrating an embodiment of the disclosure and are not to be construed as limiting the disclosure. Further objects, features and advantages of the disclosure will become apparent from the following detailed description taken in conjunction with the accompanying figures showing illustrative embodiments of the disclosure, in which:

FIGS. 1A and 1B. Illustration of general principle for bifunctional compounds. (1A) Exemplary bifunctional compounds comprise a protein targeting moiety (PTM; darkly shaded rectangle), a ubiquitin ligase binding moiety (ULM; lightly shaded triangle), and optionally a linker moiety (L; black line) coupling or tethering the PTM to the ULM. (1B) Illustrates the functional use of the bifunctional compounds as described herein. Briefly, the ULM recognizes and binds to a specific E3 ubiquitin ligase, and the PTM binds and recruits a target protein bringing it into close proximity to the E3 ubiquitin ligase. Typically, the E3 ubiquitin ligase is complexed with an E2 ubiquitin-conjugating protein, and either alone or via the E2 protein catalyzes attachment of ubiquitin (dark circles) to a lysine on the target protein via an isopeptide bond. The poly-ubiquitinated protein (far right) is then targeted for degradation by the proteosomal machinery of the cell.

FIGS. 2A, 2B, 2C, 2D, 2E, 2F, 2G, 2H, 2I, 2J, 2K and 2L. (2A) Chemical structures of exemplary compound 102, exemplary compound 106, and commercial control defactinib. (B) FAK levels in response to dose escalations of exemplary compound 100, in PC3 cell line after 24 hour treatment. (C) FAK levels in response to dose escalations of exemplary compound 101, in PC3 cell line after 24 hour treatment. (D) FAK levels in response to dose escalations of exemplary compound 102, in PC3 cell line after 24 hour treatment. (E) FAK levels in response to dose escalations of exemplary compound 103, in PC3 cell line after 24 hour treatment. (F) FAK levels in response to dose escalations of exemplary compound 104, in PC3 cell line after 24 hour treatment. (G) FAK levels in response to dose escalations of exemplary compound 105, in PC3 cell line after 24 hour treatment. (H) FAK levels in response to dose escalations of exemplary compound 106, in PC3 cell line after 24 hour treatment. (I) FAK levels in response to dose escalations of exemplary compound 107, in PC3 cell line after 24 hour treatment. (J) FAK levels in response to dose escalations of exemplary compound 108, in PC3 cell line after 24 hour treatment. (K) FAK levels in response to dose escalations of exemplary compound 109, in PC3 cell line after 24 hour treatment. (L) FAK levels in response to dose escalations of defactinib, in PC3 cell line after 24 hour treatment. Representative blots with three replicated per concentration are shown in FIGS. 2B-2L.

FIGS. 3A, 3B, and 3C. Fitted Fak inhibition data of exemplary compounds 100-109 and defactinib (n=2).

FIGS. 4A, 4B, 4C, 4D, 4E, 4F, 4G, 4H, 4G, 4H, 4I, 4J, and 4K. Fak quantification derived from western blots of FIGS. 2B-2L, respectively (n=3, error=standard deviation).

FIGS. 5A and 5B. Fitted Fak degradation data of exemplar compounds 100-105 (A) and exemplar compounds 106-109 (B). n=3, error bars=SEM.

FIGS. 6A, 6B, and 6C. Serum deprived PC3 cells were treated for 24 hours with the exemplary compound 102 (6A), exemplary compound 103 (6B), or defactinib (6C), and western blot analysis was performed, probing for p-Fak levels. Representative blots with three replicated per concentration are shown in FIGS. 6A-6C.

FIGS. 7A, 7B, and 7C. p-Fak quantification derived from the western blots of FIGS. 6A, 6B, and 6C, respectively. n=3, error=SD.

FIGS. 8A and 8B. Effects of Fak degradation (exemplary compound 102) vs. Fak inhibition (defactinib) on total Fak levels, p-Fak(Y397), p-paxillin and p-Akt(S473). 24 hour treatment in serum deprived PC3 cells. n.s. P value>0.05; * P value<0.05; ** P value<0.01; *** P value<0.001.

FIGS. 9A, 9B, 9C, 9D, 9E, and 9F. Fak signaling. Serum deprived PC3 cells were treated for 24 hours with the exemplary compound 106, exemplary compound 102, or defactinib and western blot analysis was performed, probing for the indicated proteins. Each blot resembles a biological replicate, three replicates are shown.

FIGS. 10A, 10B, 10C, and 10D. Fak signaling. Fak (A), p-Fak (B), p-Paxillin (C), p-Akt (S473) (D) quantification derived from western blots of FIGS. 9A-9F. n=3, error=SD.

FIGS. 11A and 11B. Wound healing ability of MDA-MB-231 cells in response to exemplary compound 102 treatment. (A) Dose dependent inhibition of MDA-MB-231 cell migration in response to exemplary compound 102 treatment as determined by the wound healing assay. Wounded area was captured at time 0 hour and after 24 hours. Wound healing capacity was determined by subtracting the wounded area after 24 hours from the wounded area at 0 hour. (B) Graphical representation of percent wound healing. (***P value<0.001). n=3

FIGS. 12A and 12B. Invasion dose response. (A) Dose dependent invasion of MDA-MB-231 cell in response to exemplary compound 102 treatment as determined by the transwell assay. Cells were fixed, permeabilized and stained with crystal violet and examined under a light microscope. Invaded area was captured and cells quantified by counting after 24 hours. (B) Graphical representation of rel. invasion. (***P value<0.001). n=3

FIGS. 13A and 13B. Wound healing assay. (A) Effects of exemplary compound 102 and defactinib on wound healing of MDA-MB-231 cells. Wounded area was captured just after wound introduction and after 24 hours of treatment. (B) Graphical representation of percent wound healing. n.s. P value>0.05; ***P value<0.001. n=3.

FIG. 14. Transwell cell invasion. Invasion of MDA-MB-231 cell in response to exemplary compound 102 and defactinib treatment (100 nM) as determined by transwell assay. Cells were fixed, permeabilized and stained with crystal violet and examined under a light microscope. Invaded area was captured and cells quantified by counting after 24 hours. Graphical representation of relative invasion. n.s. P value>0.05; ***P value<0.001. n=3.

FIGS. 15A and 15B. Validation of RPPA-AR results. (A) Western blot analysis of dose dependent incubation of MDB-MD-231 cells in the presence of 10% FBS with defactinib or exemplary compound 102, respectively. (B) Quantification of western blots from FIG. 15A. n=1.

FIGS. 16A and 16B. Validation of RPPA-p-Akt(S473) results. (A) Western blot analysis of dose dependent incubation of MDB-MD-231 cells in the presence of 10% FBS with defactinib or exemplary compound 102, respectively. (B) Quantification of western blots from FIG. 16A. n=1.

FIGS. 17A and 17B. Validation of RPPA-p-Src(Y527) results. (A) Western blot analysis of dose dependent incubation of MDB-MD-231 cells in the presence of 10% FBS with defactinib or exemplary compound 102, respectively. (B) Quantification of western blots from FIG. 17A. n=1.

FIGS. 18A and 18B. Validation of RPPA-p-S6RP(S240/S244) results. (A) Western blot analysis of dose dependent incubation of MDB-MD-231 cells in the presence of 10% FBS with defactinib or exemplary compound 102, respectively. (B) Quantification of western blots from FIG. 18A. n=1

FIGS. 19A and 19B. S6RP total levels. (A) Western blot analysis of dose dependent incubation of MDB-MD-231 cells in the presence of 10% FBS with defactinib or exemplary compound 102, respectively. (B) Quantification of western blots from FIG. 19A. n=1

FIG. 20A. Illustrates the pharmacokinetics for intraperitoneally and intravenously injected exemplary compound 103 in CD1 mice.

FIG. 20B. Table 9 includes pharmacokinetic data for exemplary compound 103 in CD1 mice.

FIG. 21A. Illustrates the pharmacokinetics for intraperitoneally and intravenously injected exemplary compound 107 in CD1 mice.

FIG. 21B. Table 12 includes pharmacokinetic data for exemplary compound 107 in CD1 mice.

FIG. 22A. Illustrates the pharmacokinetics for intraperitoneally and intravenously injected exemplary compound 100 in CD1 mice.

FIG. 22B. Table 15 includes pharmacokinetic data for exemplary compound 100 in CD1 mice.

FIG. 23A. Illustrates the pharmacokinetics for intraperitoneally and intravenously injected exemplary compound 101 in CD1 mice.

FIG. 23B. Table 18 includes pharmacokinetic data for exemplary compound 101 in CD1 mice.

FIG. 24A. Illustrates the pharmacokinetics for intraperitoneally and intravenously injected exemplary compound 102 in CD1 mice.

FIG. 24B. Table 21 includes pharmacokinetic data for exemplary compound 102 in CD1 mice.

FIGS. 25A, 25B, and 25C. Cell proliferation MDA-MB-231 cells. Dose dependent proliferation of MDA-MB-231 cell in response to exemplary compounds of the present disclosure and defactinib treatment after 96 hours. MTS assay; n=3.

FIGS. 26A, 26B, and 26C. Cell proliferation PC3 cells. Dose dependent proliferation of MDA-MB-231 cell in response to exemplary compounds and defactinib treatment after 96 h. MTS assay; n=3.

### DETAILED DESCRIPTION

The following is a detailed description provided to aid those skilled in the art in practicing the present disclosure. Those of ordinary skill in the art may make modifications and variations in the embodiments described herein without departing from the spirit or scope of the present disclosure. All publications, patent applications, patents, figures and other references mentioned herein are expressly incorporated by reference in their entirety.

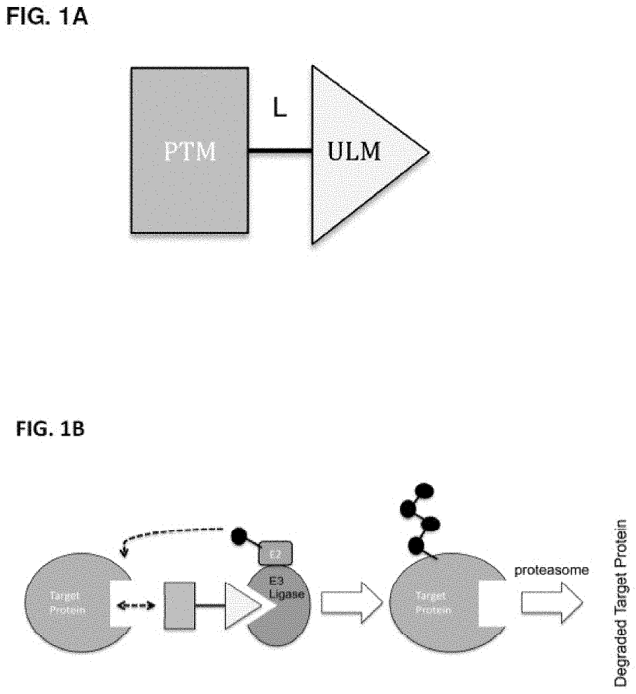

Presently described are compositions and methods that relate to the surprising and unexpected discovery that an E3 ubiquitin ligase protein (e.g., inhibitors of apoptosis proteins (IAP), a Von Hippel-Lindau E3 ubiquitin ligase (VHL), a cereblon E3 ubiquitin ligase, or a mouse double minute 2 homolog (MDM2) E3 ubiquitin ligase) ubiquitinates a target protein once it and the target protein are placed in proximity by a bifunctional or chimeric construct that binds the E3 ubiquitin ligase protein and the target protein. Accordingly the present disclosure provides such compounds and compositions comprising an E3 ubiquintin ligase binding moiety (“ULM”) coupled to a protein target binding moiety (“PTM”), which result in the ubiquitination of a chosen target protein, which leads to degradation of the target protein by the proteasome (see FIG. 1). The present disclosure also provides a library of compositions and the use thereof.

In certain aspects, the present disclosure provides compounds which comprise a ligand, e.g., a small molecule ligand (i.e., having a molecular weight of below 2,000, 1,000, 500, or 200 Daltons), which is capable of binding to a ubiquitin ligase, such as IAP, VHL, MDM2, or cereblon. The compounds also comprise a moiety that is capable of binding to target protein, in such a way that the target protein is placed in proximity to the ubiquitin ligase to effect degradation (and/or inhibition) of that protein. Small molecule can mean, in addition to the above, that the molecule is non-peptidyl, that is, it is not generally considered a peptide, e.g., comprises fewer than 4, 3, or 2 amino acids. In accordance with the present description, the PTM, ULM or bifunctional molecule of the present disclosure can be a small molecule.

Unless otherwise defined, all technical and scientific terms used herein have the same meaning as commonly understood by one of ordinary skill in the art to which this disclosure belongs. The terminology used in the description is for describing particular embodiments only and is not intended to be limiting of the disclosure.

Where a range of values is provided, it is understood that each intervening value, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either both of those included limits are also included in the disclosure.

The following terms are used to describe the present disclosure. In instances where a term is not specifically defined herein, that term is given an art-recognized meaning by those of ordinary skill applying that term in context to its use in describing the present disclosure.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, in certain methods described herein that include more than one step or act, the order of the steps or acts of the method is not necessarily limited to the order in which the steps or acts of the method are recited unless the context indicates otherwise.

The terms “co-administration” and “co-administering” or “combination therapy” refer to both concurrent administration (administration of two or more therapeutic agents at the same time) and time varied administration (administration of one or more therapeutic agents at a time different from that of the administration of an additional therapeutic agent or agents), as long as the therapeutic agents are present in the patient to some extent, preferably at effective amounts, at the same time. In certain preferred aspects, one or more of the present compounds described herein, are coadministered in combination with at least one additional bioactive agent, especially including an anticancer agent. In particularly preferred aspects, the co-administration of compounds results in synergistic activity and/or therapy, including anticancer activity.

The term “compound”, as used herein, unless otherwise indicated, refers to any specific chemical compound disclosed herein and includes tautomers, regioisomers, geometric isomers, and where applicable, stereoisomers, including optical isomers (enantiomers) and other stereoisomers (diastereomers) thereof, as well as pharmaceutically acceptable salts and derivatives, including prodrug and/or deuterated forms thereof where applicable, in context. Deuterated small molecules contemplated are those in which one or more of the hydrogen atoms contained in the drug molecule have been replaced by deuterium.

Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds. The term also refers, in context to prodrug forms of compounds which have been modified to facilitate the administration and delivery of compounds to a site of activity. It is noted that in describing the present compounds, numerous substituents and variables associated with same, among others, are described. It is understood by those of ordinary skill that molecules which are described herein are stable compounds as generally described hereunder. When the bond is shown, both a double bond and single bond are represented or understood within the context of the compound shown and well-known rules for valence interactions.

The term “ubiquitin ligase” refers to a family of proteins that facilitate the transfer of ubiquitin to a specific substrate protein, targeting the substrate protein for degradation. For example, IAP an E3 ubiquitin ligase protein that alone or in combination with an E2 ubiquitin-conjugating enzyme causes the attachment of ubiquitin to a lysine on a target protein, and subsequently targets the specific protein substrates for degradation by the proteasome. Thus, E3 ubiquitin ligase alone or in complex with an E2 ubiquitin conjugating enzyme is responsible for the transfer of ubiquitin to targeted proteins. In general, the ubiquitin ligase is involved in polyubiquitination such that a second ubiquitin is attached to the first; a third is attached to the second, and so forth. Polyubiquitination marks proteins for degradation by the proteasome. However, there are some ubiquitination events that are limited to mono-ubiquitination, in which only a single ubiquitin is added by the ubiquitin ligase to a substrate molecule. Mono-ubiquitinated proteins are not targeted to the proteasome for degradation, but may instead be altered in their cellular location or function, for example, via binding other proteins that have domains capable of binding ubiquitin. Further complicating matters, different lysines on ubiquitin can be targeted by an E3 to make chains. The most common lysine is Lys48 on the ubiquitin chain. This is the lysine used to make polyubiquitin, which is recognized by the proteasome.

The term “patient” or “subject” is used throughout the specification to describe an animal, preferably a human or a domesticated animal, to whom treatment, including prophylactic treatment, with the compositions according to the present disclosure is provided. For treatment of those infections, conditions or disease states which are specific for a specific animal such as a human patient, the term patient refers to that specific animal, including a domesticated animal such as a dog or cat or a farm animal such as a horse, cow, sheep, etc. In general, in the present disclosure, the term patient refers to a human patient unless otherwise stated or implied from the context of the use of the term.

The term “effective” is used to describe an amount of a compound, composition or component which, when used within the context of its intended use, effects an intended result. The term effective subsumes all other effective amount or effective concentration terms, which are otherwise described or used in the present application.

Compounds and Compositions

In one aspect, the description provides compounds comprising an E3 ubiquitin ligase binding moiety (“ULM”) that is an IAP E3 ubiquitin ligase binding moiety (an “ILM”), a cereblon E3 ubiquitin ligase binding moiety (a “CLM”), a Von Hippel-Lindae E3 ubiquitin ligase (VHL) binding moiety (VLM), and/or a mouse double minute 2 homologue (MDM2) E3 ubiquitin ligase binding moiety (MLM). In an exemplary embodiment, the ULM is coupled to a target protein binding moiety (PTM) via a chemical linker (L) according to the structure:

(A) PTM-L-ULM

wherein L is a bond or a chemical linker group, ULM is a E3 ubiquitin ligase binding moiety, and PTM is a target protein binding moiety. The number and/or relative positions of the moieties in the compounds illustrated herein is provided by way of example only. As would be understood by the skilled artisan, compounds described herein can be synthesized with any desired number and/or relative position of the respective functional moieties.

The terms ULM, ILM, VLM, MLM, and CLM are used in their inclusive sense unless the context indicates otherwise. For example, the term ULM is inclusive of all ULMs, including those that bind IAP (i.e., ILMs), MDM2 (i.e., MLM), cereblon (i.e., CLM), and VHL (i.e., VLM). Further, the term ILM is inclusive of all possible IAP E3 ubiquitin ligase binding moieties, the term MLM is inclusive of all possible MDM2 E3 ubiquitin ligase binding moieties, the term VLM is inclusive of all possible VHL binding moieties, and the term CLM is inclusive of all cereblon binding moieties.

In another aspect, the present disclosure provides bifunctional or multifunctional compounds useful for regulating protein activity by inducing the degradation of a target protein. In certain embodiments, the compound comprises an ILM or a VLM or a CLM or a MLM coupled, e.g., linked covalently, directly or indirectly, to a moiety that binds a target protein (i.e., a protein targeting moiety or a “PTM”). In certain embodiments, the ILM/VLM/CLM/MLM and PTM are joined or coupled via a chemical linker (L). The ILM binds the IAP E3 ubiquitin ligase, the VLM binds VHL, CLM binds the cereblon E3 ubiquitin ligase, and MLM binds the MDM2 E3 ubiquitin ligase, and the PTM recognizes a target protein and the interaction of the respective moieties with their targets facilitates the degradation of the target protein by placing the target protein in proximity to the ubiquitin ligase protein. An exemplary bifunctional compound can be depicted as:

(B) PTMILM

(C) PTMCLM

(D) PTMVLM

(E) PTMMLM

In certain embodiments, the bifunctional compound further comprises a chemical linker (“L”). For example, the bifunctional compound can be depicted as:

(F) PTMLILM

(G) PTMLCLM

(H) PTMLVLM

(I) PTMLMLM

wherein the PTM is a protein/polypeptide targeting moiety, the L is a chemical linker, the ILM is a IAP E3 ubiquitin ligase binding moiety, the CLM is a cereblon E3 ubiquitin ligase binding moiety, the VLM is a VHL binding moiety, and the MLM is a MDM2 E3 ubiquitin ligase binding moiety.

In certain embodiments, the ULM (e.g., a ILM, a CLM, a VLM, or a MLM) shows activity or binds to the E3 ubiquitin ligase (e.g., IAP E3 ubiquitin ligase, cereblon E3 ubiquitin ligase, VHL, or MDM2 E3 ubiquitin ligase) with an IC₅₀ of less than about 200 μM. The IC₅₀ can be determined according to any method known in the art, e.g., a fluorescent polarization assay.

In certain additional embodiments, the bifunctional compounds described herein demonstrate an activity with an IC₅₀ of less than about 100, 50, 10, 1, 0.5, 0.1, 0.05, 0.01, 0.005, 0.001 mM, or less than about 100, 50, 10, 1, 0.5, 0.1, 0.05, 0.01, 0.005, 0.001 μM, or less than about 100, 50, 10, 1, 0.5, 0.1, 0.05, 0.01, 0.005, 0.001 nM, or less than about 100, 50, 10, 1, 0.5, 0.1, 0.05, 0.01, 0.005, 0.001 pM.

In certain embodiments, the compounds as described herein comprise multiple PTMs (targeting the same or different protein targets), multiple ULMs, one or more ULMs (i.e., moieties that bind specifically to multiple/different E3 ubiquitin ligase, e.g., VHL, IAP, cereblon, and/or MDM2) or a combination thereof. In any of the aspects or embodiments described herein, the PTMs and ULMs (e.g., ILM, VLM, CLM, and/or MLM) can be coupled directly or via one or more chemical linkers or a combination thereof. In additional embodiments, where a compound has multiple ULMs, the ULMs can be for the same E3 ubiquintin ligase or each respective ULM can bind specifically to a different E3 ubiquitin ligase. In still further embodiments, where a compound has multiple PTMs, the PTMs can bind the same target protein or each respective PTM can bind specifically to a different target protein.

In certain embodiments, where the compound comprises multiple ULMs, the ULMs are identical. In additional embodiments, the compound comprising a plurality of ULMs (e.g., ULM, ULM′, etc.), at least one PTM coupled to a ULM directly or via a chemical linker (L) or both. In certain additional embodiments, the compound comprising a plurality of ULMs further comprises multiple PTMs. In still additional embodiments, the PTMs are the same or, optionally, different. In still further embodiments, wherein the PTMs are different, the respective PTMs may bind the same protein target or bind specifically to a different protein target.

In certain embodiments, the compound may comprise a plurality of ULMs and/or a plurality of ULM's. In further embodiments, the compound comprising at least two different ULMs, a plurality of ULMs, and/or a plurality of ULM's further comprises at least one PTM coupled to a ULM or a ULM′ directly or via a chemical linker or both. In any of the embodiments described herein, a compound comprising at least two different ULMs can further comprise multiple PTMs. In still additional embodiments, the PTMs are the same or, optionally, different. In still further embodiments, wherein the PTMs are different the respective PTMs may bind the same protein target or bind specifically to a different protein target. In still further embodiments, the PTM itself is a ULM (or ULM′), such as an ILM, a VLM, a CLM, a MLM, an ILM′, a VLM′, a CLM′, and/or a MLM′.

In additional embodiments, the description provides the compounds as described herein including their enantiomers, diastereomers, solvates and polymorphs, including pharmaceutically acceptable salt forms thereof, e.g., acid and base salt forms.

Exemplary ILMs

AVPI Tetrapeptide Fragments

In any of the compounds described herein, the ILM can comprise an alanine-valine-proline-isoleucine (AVPI) tetrapeptide fragment or an unnatural mimetic thereof. In certain embodiments, the ILM is selected from the group consisting of chemical structures represented by Formulas (I), (II), (III), (IV), and (V):

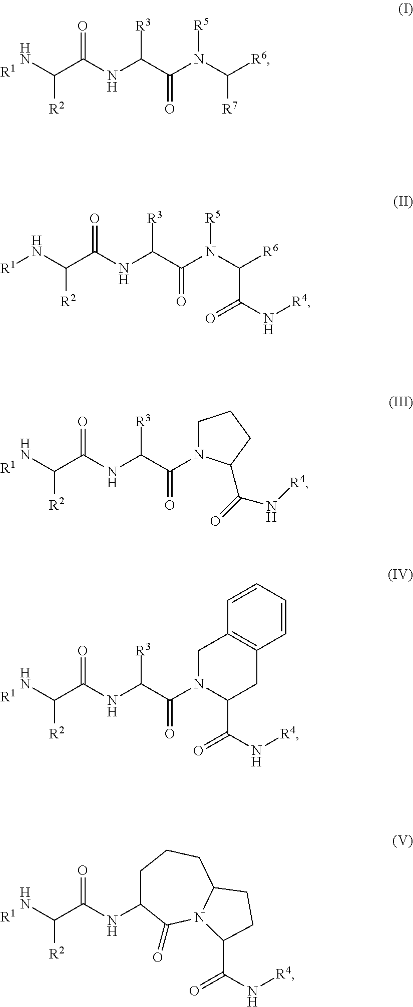

wherein:
R¹ for Formulas (I), (II), (III), (IV), and (V) is selected from H or alkyl;
R² for Formulas (I), (II), (III), (IV), and (V) is selected from H or alkyl;
R³ for Formulas (I), (II), (III), (IV), and (V) is selected from H, alkyl, cycloalkyl and heterocycloalkyl;
R⁵ and R⁶ for Formulas (I), (II), (III), (IV), and (V) are independently selected from H, alkyl, cycloalkyl, heterocycloalkyl, or more preferably, R⁵ and R⁶ taken together for Formulas (I), (II), (III), (IV), and (V) form a pyrrolidine or a piperidine ring further optionally fused to 1-2 cycloalkyl, heterocycloalkyl, aryl or heteroaryl rings, each of which can then be further fused to another cycloalkyl, heterocycloalkyl, aryl or heteroaryl ring;
R³ and R⁵ for Formulas (I), (II), (III), (IV), and (V) taken together can form a 5-8-membered ring further optionally fused to 1-2 cycloalkyl, heterocycloalkyl, aryl or heteroaryl rings;
R⁷ for Formulas (I), (II), (III), (IV), and (V) is selected from cycloalkyl, cycloalkylalkyl, heterocycloalkyl, heterocycloalkylalkyl, aryl, aryl-C(O)—R⁴, arylalkyl, heteroaryl, heteroaryl-C(O)—R⁴, heteroaryl-R⁴, heteroaryl-naphthalene, heteroarylalkyl, or C(O)NH—R⁴, each one further optionally substituted with 1-3 substituents selected from halogen, alkyl, haloalkyl, hydroxyl, alkoxy, cyano, (hetero)cycloalkyl, (hetero)aryl, —C(O)NH—R⁴, or —C(O)—R⁴; and
R⁴ is selected from alkyl, cycloalkyl, heterocycloalkyl, cycloalkylalkyl, heterocycloalkylalkyl, aryl, arylalkyl, heteroaryl, heteroarylalkyl, further optionally substituted with 1-3 substituents as described above.

As shown above, P1, P2, P3, and P4 of Formular (II) correlate with A, V, P, and I, respectively, of the AVPI tetrapeptide fragment or an unnatural mimetic thereof. Similarly, each of Formulas (I) and (III) through (V) have portions correlating with A, V, P, and I of the AVPI tetrapeptide fragment or an unnatural mimetic thereof.

In any of the compounds described herein, the ILM can have the structure of Formula (VI), which is a derivative of IAP antagonists described in WO Pub. No. 2008/014236, or an unnatural mimetic thereof:

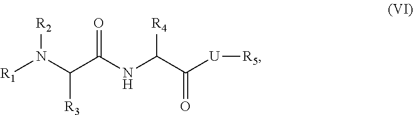

wherein:
R₁ of Formula (VI) is, independently selected from H, C₁-C₄-alky, C₁-C₄-alkenyl, C₁-C₄-alkynyl or C₃-C₁₀-cycloalkyl which are unsubstituted or substituted;
R₂ of Formula (VI) is, independently selected from H, C₁-C₄-alkyl, C₁-C₄-alkenyl, C₁-C₄-alkynyl or C₃-C₁₀-cycloalkyl which are unsubstituted or substituted;
R₃ of Formula (VI) is, independently selected from H, —CF₃, —C₂H₅, C₁-C₄-alkyl, C₁-C₄-alkenyl, C₁-C₄-alkynyl, —CH₂—Z or any R₂ and R₃ together form a heterocyclic ring;
each Z of Formula (VI) is, independently selected from H, —OH, F, Cl, —CH₃, —CF₃, —CH₂Cl, —CH₂F or —CH₂OH;
R₄ of Formula (VI) is, independently selected from C₁-C₁₆ straight or branched alkyl, C₁-C₁₆-alkenyl, C₁-C₁₆-alkynyl, C₃-C₁₀-cycloalkyl, —(CH₂)₀₋₆—Z₁, —(CH₂)₀₋₆-aryl, and —(CH₂)₀₋₆-het, wherein alkyl, cycloalkyl, and phenyl are unsubstituted or substituted;
R₅ of Formula (VI) is, independently selected from H, C₁₋₁₀-alkyl, aryl, phenyl, C₃₋₇-cycloalkyl, —(CH₂)₁₋₆—C₃₋₇-cycloalkyl, —C₁₋₁₀-alkyl-aryl, —(CH₂)₀₋₆—C₃₋₇-cycloalkyl-(CH₂)₀₋₆-phenyl, —(CH₂)₀₋₄—CH[(CH₂)₁₋₄-phenyl]₂, indanyl, —C(O)—C₁₋₁₀-alkyl, —C(O)—(CH₂)₁₋₆—C₃₋₇-cycloalkyl, —C(O)—(CH₂)₀₋₆-phenyl, —(CH₂)₀₋₆—C(O)-phenyl, —(CH₂)₀₋₆-het, —C(O)—(CH₂)₁₋₆-het, or R₅ is selected from a residue of an amino acid, wherein the alkyl, cycloalkyl, phenyl, and aryl substituents are unsubstituted or substituted;
Z₁ of Formula (VI) is, independently selected from —N(R₁₀)—C(O)—C₁₋₁₀-alkyl, —N(R₁₀)—C(O)—(CH₂)₀₋₆—C₃₋₇-cycloalkyl, —N(R₁₀)—C(O)—(CH₂)₀₋₆-phenyl, —N(R₁₀)—C(O)(CH₂)₁₋₆-het, —C(O)—N(R₁₁)(R₁₂), —C(O)—O—C₁₋₁₀-alkyl, —C(O)—O—(CH₂)₁₋₆—C₃₋₇-cycloalkyl, —C(O)—O—(CH₂)₀₋₆-phenyl, —C(O)—O—(CH₂)₁₋₆-het, —O—C(O)—C₁₋₁₀-alkyl, —O—C(O)—(CH₂)₁₋₆—C₃₋₇-cycloalkyl, —O—C(O)—(CH₂)₀₋₆-phenyl, —O—C(O)—(CH₂)₁₋₆-het, wherein alkyl, cycloalkyl, and phenyl are unsubstituted or substituted;
het of Formula (VI) is, independently selected from a 5-7 member heterocyclic ring containing 1-4 heteroatoms selected from N, O, and S, or an 8-12 member fused ring system including at least one 5-7 member heterocyclic ring containing 1, 2, or 3 heteroatoms selected from N, O, and S, which heterocyclic ring or fused ring system is unsubstituted or substituted on a carbon or nitrogen atom;
R₁₀ of Formula (VI) is selected from H, —CH₃, —CF₃, —CH₂OH, or —CH₂Cl;
R₁₁ and R₁₂ of Formula (VI) are independently selected from H, C₁₋₄-alkyl, C₃₋₇-cycloalkyl, —(CH₂)₁₋₆—C₃₋₇-cycloakyl, (CH₂)₀₋₆-phenyl, wherein alkyl, cycloalkyl, and phenyl are unsubstituted or substituted; or R₁₁ and R₁₂ together with the nitrogen form het, and U of Formula (VI) is, independently, as shown in Formula (VII):

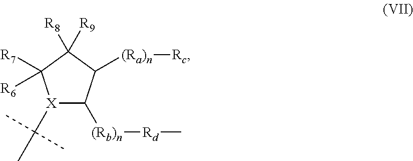

wherein:
each n of Formula (VII) is, independently selected from 0 to 5;
X of Formula (VII) is selected from the group —CH and N;
Rₐ and R_{b}, of Formula (VII) are independently selected from the group O, S, or N atom or C₀₋₈-alkyl wherein one or more of the carbon atoms in the alkyl chain are optionally replaced by a heteroatom selected from O, S, or N, and where each alkyl is, independently, either unsubstituted or substituted;
R_{d} of Formula (VII) is selected from the group Re-Q-(R_{f})ₚ(R_{g})_{q}, and Ar₁-D-Ar₂:
R_{c} of Formula (VII) is selected from the group H or any R_{c} and R_{d} together form a cycloalkyl or het; where if R_{c} and R_{d} form a cycloalkyl or het, R₅ is attached to the formed ring at a C or N atom;
p and q of Formula (VII) are independently selected from 0 or 1;
Rₑ of Formula (VII) is selected from the group C₁₋₈-alkyl and alkylidene, and each Rₑ is either unsubstituted or substituted;
Q is selected from the group N, O, S, S(O), and S(O)₂;
Ar₁ and Ar₂ of Formula (VII) are independently selected from the group of substituted or unsubstituted aryl and het;
R_{f} and R_{g} of Formula (VII) are independently selected from H, —C₁₋₁₀-alkyl, C₁₋₁₀-alkylaryl, —OH, —O—C₁₋₁₀-alkyl, —(CH₂)₀₋₆—C₃₋₇-cycloalky, —O—(CH₂)₀₋₆-aryl, phenyl, aryl, phenyl-phenyl, —(CH₂)₁₋₆-het, —O—(CH₂)₁₋₆-het, —OR₁₃, —C(O)—R₁₃, —C(O)—N(R₁₃)(R₁₄), —N(R₁₃)(R₁₄), —S—R₁₃, —S(O)—R₁₃, —S(O)₂—R₁₃, —S(O)₂—NR₁₃R₁₄, —NR₁₃—S(O)₂—R₁₄, —S—Cₜ₋₁₀-alkyl, aryl-C₁₋₄-alkyl, or het-C₁₋₄-alkyl, wherein alkyl, cycloalkyl, het, and aryl are unsubstituted or substituted, —SO₂—C₁₋₂-alkyl, —SO₂—C₁₋₂-alkylphenyl, —O—C₁₋₄-alkyl, or any R_{g} and R_{f} together form a ring selected from het or aryl;
D of Formula (VII) is selected from the group —CO—, —C(O)—C₁₋₇-alkylene or arylene, —CF₂—, —O—, —S(O)ᵣ where r is 0-2, 1,3-dioxalane, or C₁₋₇-alkyl-OH; where alkyl, alkylene, or arylene are unsubstituted or substituted with one or more halogens, OH, —O—C₁₋₆-alkyl, —S—C₁₋₆-alkyl, or —CF₃; or each D is, independently selected from N(Rₕ);
Rₕ is selected from the group H, unsubstituted or substituted C₁₋₇-alkyl, aryl, unsubstituted or substituted —O—(C₁₋₇-cycloalkyl), —C(O)—C₁₋₁₀-alkyl, —C(O)—C₀₋₁₁-alkyl-aryl, —C—O—C₀₁₋₁₀-alkyl, —C—O—C₀₋₁₀-alkyl-aryl, —SO₂—C₁₋₁₀-alkyl, or —SO₂—(C₀₋₁₀-alkylaryl);
R₆, R₇, R₈, and R₉ of Formula (VII) are, independently, selected from the group H, —C₁₋₁₀-alkyl, —C₁₋₁₀-alkoxy, aryl-C₁₋₁₀-alkoxy, —OH, —O—C₁₋₁₀-alkyl, —(CH₂)₀₋₆—C₃₋₇-cycloalkyl, —O—(CH₂)₀₋₆-aryl, phenyl, —(CH₂)₁₋₆-het, —O—(CH₂)₁₋₆-het, —OR₁₃, —C(O)—R₁₃, —C(O)—N(R₁₃)(R₁₄), —N(R₁₃)(R₁₄), —S—R₁₃, —S(O)—R₁₃, —S(O)₂—R₁₃, —S(O)₂—NR₁₃R₁₄, or —NR₁₃—S(O)₂—R₁₄; wherein each alkyl, cycloalkyl, and aryl is unsubstituted or substituted; and any R₆, R₇, R₈, and R₉ optionally together form a ring system;
R₁₃ and R₁₄ of Formula (VII) are independently selected from the group H, C₁₋₁₀-alkyl, —(CH₂)₀₋₆—C₃₋₇-cycloalkyl, —(CH₂)₀₋₆—(CH)₀₋₁-(aryl)₁₋₂, —C(O)—C₁₋₁₀-alkyl, —C(O)—(CH₂)₁₋₆—C₃₋₇-cycloalkyl, —C(O)—O—(CH₂)₀₋₆-aryl, —C(O)—(CH₂)₀₋₆—O-fluorenyl, —C(O)—NH—(CH₂)₀₋₆-aryl, —C(O)—(CH₂)₀₋₆-aryl, —C(O)—(CH₂)₀₋₆-het, —C(S)—C₁₋₁₀-alkyl, —C(S)—(CH₂)₁₋₆—C₃₋₇-cycloalkyl, —C(S)—O—(CH₂)₀₋₆-aryl, —C(S)—(CH₂)₀₋₆—O-fluorenyl, —C(S)—NH—(CH₂)₀₋₆-aryl, —C(S)—(CH₂)₀₋₆-aryl, or —C(S)—(CH₂)₁₋₆-het, wherein each alkyl, cycloalkyl, and aryl is unsubstituted or substituted: or any R₁₃ and R₁₄ together with a nitrogen atom form het;
wherein alkyl substituents of R₁₃ and R₁₄ of Formula (VII) are unsubstituted or substituted and when substituted, are substituted by one or more substituents selected from C₁₋₁₀-alkyl, halogen, OH, —O—C₁₋₆-alkyl, —S—C₁₋₆-alkyl, and —CF₃; and substituted phenyl or aryl of R₁₃ and R₁₄ are substituted by one or more substituents selected from halogen, hydroxyl, C₁₋₄-alkyl, C₁₋₄-alkoxy, nitro, —CN, —O—C(O)—C₁₋₄-alkyl, and —C(O)—O—C₁₋₄-aryl; or a pharmaceutically acceptable salt or hydrate thereof.

In any of the compounds described herein, the ILM can have the structure of Formula (VIII), which is based on the IAP ligrands described in Ndubaku, C., et al. Antagonism of c-IAP and XIAP proteins is required for efficient induction of cell death by small-molecule IAP antagonists, ACS Chem. Biol., 557-566, 4 (7) (2009), or an unnatural mimetic thereof:

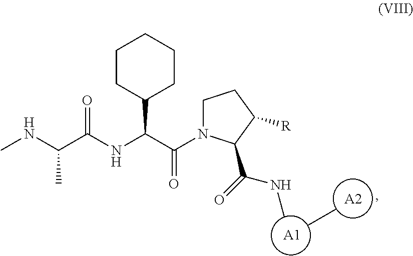

wherein each of A1 and A2 of Formula (VIII) is independently selected from optionally substituted monocyclic, fused rings, aryls and heteroaryls; and

R of Formula (VIII) is selected from H or Me.

In a particular embodiment, the linker group L is attached to A1 of Formula (VIII). In another embodiment, the linker group L is attached to A2 of Formula (VIII).

In a particular embodiment, the ILM is selected from the group consisting of

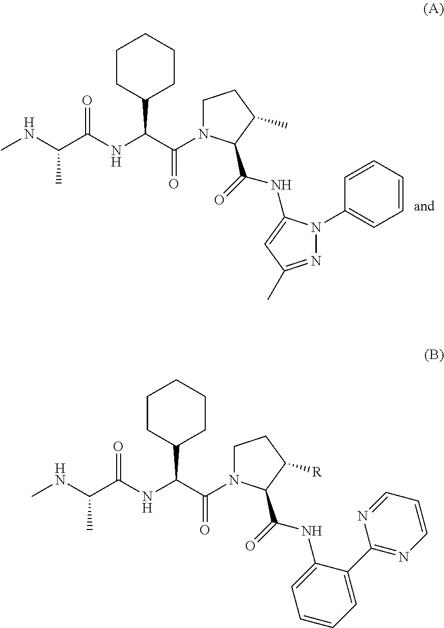

In any of the compounds described herein, the ILM can have the structure of Formula (IX), which is derived from the chemotypes cross-referenced in Mannhold, R., et al. IAP antagonists: promising candidates for cancer therapy, Drug Discov. Today, 15 (5-6), 210-9 (2010), or an unnatural mimetic thereof:

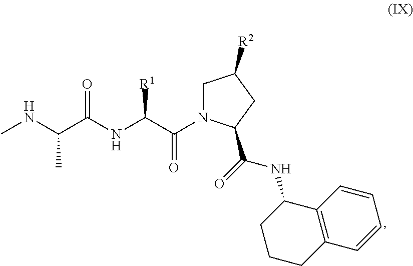

wherein R¹ is selected from alkyl, cycloalkyl and heterocycloalkyl and, most preferably, from isopropyl, tert-butyl, cyclohexyl and tetrahydropyranyl, and R² of Formula (IX) is selected from —OPh or H.

In any of the compounds described herein, the ILM can have the structure of Formula (X), which is derived from the chemotypes cross-referenced in Mannhold, R., et al. IAP antagonists: promising candidates for cancer therapy, Drug Discov. Today, 15 (5-6), 210-9 (2010), or an unnatural mimetic thereof:

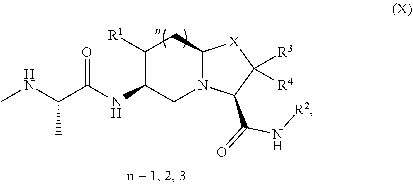

wherein:

R¹ of Formula (X) is selected from H, —CH₂OH, —CH₂CH₂OH, CH₂NH₂, —CH₂CH₂NH₂;

X of Formula (X) is selected from S or CH₂;

R² of Formula (X) is selected from:

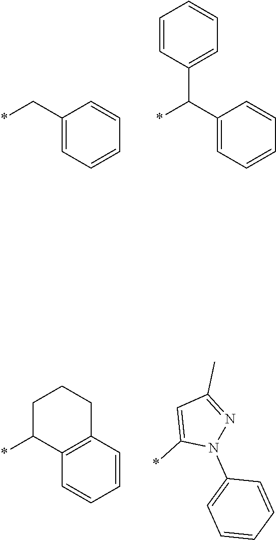

R³ and R⁴ of Formula (X) are independently selected from H or Me

In any of the compounds described herein, the ILM can have the structure of Formula (XI), which is derived from the chemotypes cross-referenced in Mannhold, R., et al. IAP antagonists: promising candidates for cancer therapy, Drug Discov. Today, 15 (5-6), 210-9 (2010), or an unnatural mimetic thereof:

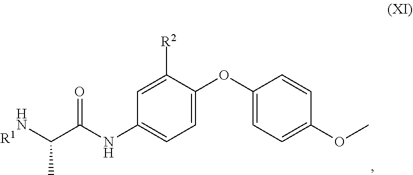

wherein R¹ of Formula (XI) is selected from H or Me, and R² of Formula (XI) is selected from H or

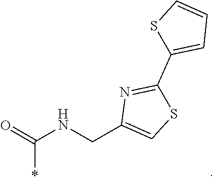 [SMILES: *C(=O)NCc1csc(-c2cccs2)n1]

In any of the compounds described herein, the ILM can have the structure of Formula (XII), which is derived from the chemotypes cross-referenced in Mannhold, R., et al. IAP antagonists: promising candidates for cancer therapy, Drug Discov. Today, 15 (5-6), 210-9 (2010), or an unnatural mimetic thereof:

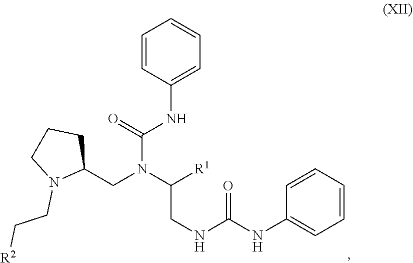

wherein:
R¹ of Formula (XII) is selected from:

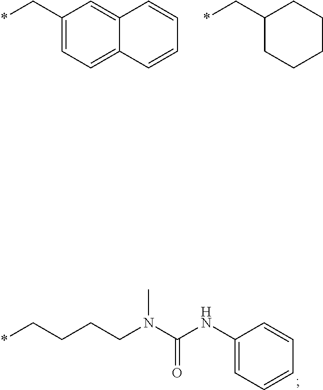

R² of Formula (XII) is selected from:

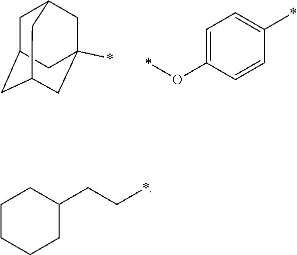

In any of the compounds described herein, the IAP E3 ubiquitin ligase binding moiety is selected from the group consisting of:

In any of the compounds described herein, the ILM can have the structure of Formula (XIII), which is based on the IAP ligands summarized in Flygare, J. A., et al. Small-molecule pan-IAP antagonists: a patent review, Expert Opin. Ther. Pat., 20 (2), 251-67 (2010), or an unnatural mimetic thereof:

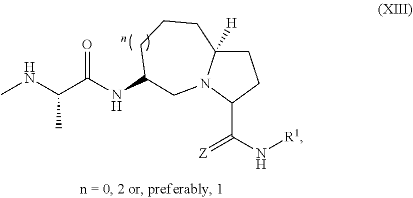

wherein:

Z of Formula (XIII) is absent or O;

R¹ of Formula (XIII) is selected from:

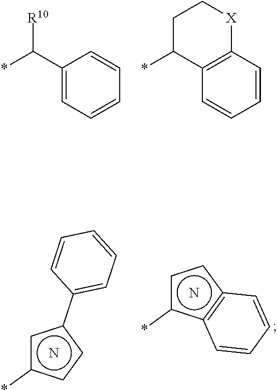

R¹⁰ of

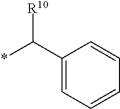

is selected from H, alkyl, or aryl;

X is selected from CH2 and O; and

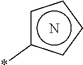

is a nitrogen-containing heteroaryl.

In any of the compounds described herein, the ILM can have the structure of Formula (XIV), which is based on the IAP ligands summarized in Flygare, J. A., et al. Small-molecule pan-IAP antagonists: a patent review, Expert Opin. Ther. Pat., 20 (2), 251-67 (2010), or an unnatural mimetic thereof:

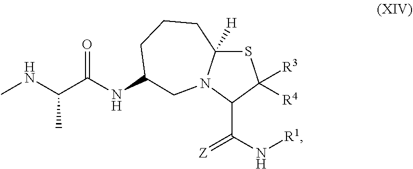

wherein:

Z of Formula (XIV) is absent or O;

R³ and R⁴ of Formula (XIV) are independently selected from H or Me;

R¹ of Formula (XIV) is selected from:

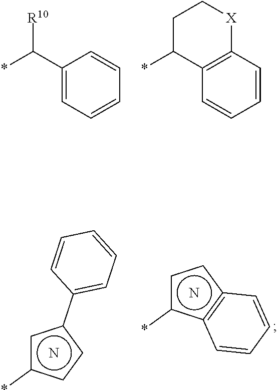

R¹⁰ of

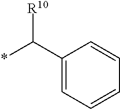

is selected from H, alkyl, or aryl;

X of

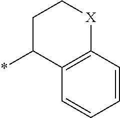

is selected from CH₂ and O; and

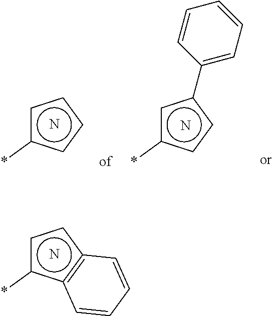

is a nitrogen-containing heteraryl.

In any of the compounds described herein, the ILM is selected from the group consisting of:

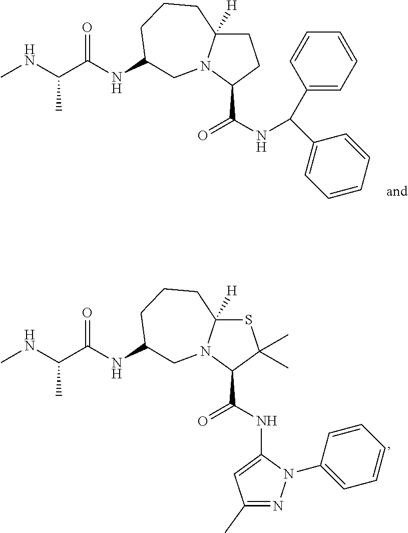

which are derivatives of ligands disclose in US Patent Pub. No. 2008/0269140 and U.S. Pat. No. 7,244,851.

In any of the compounds described herein, the ILM can have the structure of Formula (XV), which was a derivative of the IAP ligand described in WO Pub. No. 2008/128171, or an unnatural mimetic thereof:

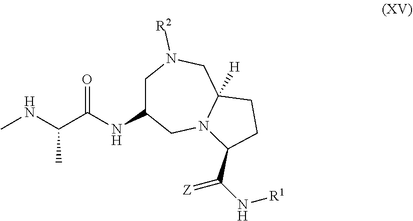

wherein:

Z of Formula (XV) is absent or O;

R¹ of Formula (XV) is selected from:

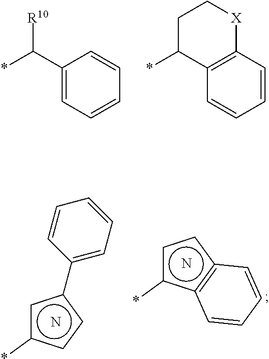

R¹⁰ of

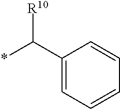

is selected from H, alkyl, or aryl;

X of

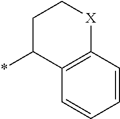

is selected from CH₂ and O; and

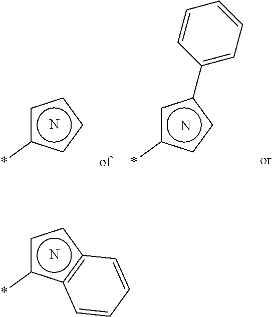

is a nitrogen-containing heteraryl; and
R² of Formula (XV) selected from H, alkyl, or acyl;

In a particular embodiment, the ILM has the following structure:

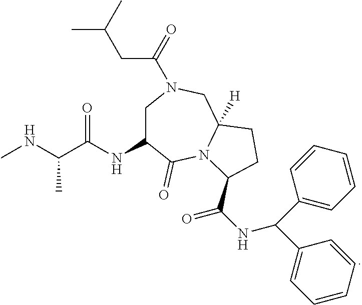

In any of the compounds described herein, the ILM can have the structure of Formula (XVI), which is based on the IAP ligand described in WO Pub. No. 2006/069063, or an unnatural mimetic thereof:

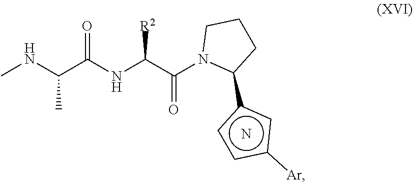

wherein:
R² of Formula (XVI) is selected from alkyl, cycloalkyl and heterocycloalkyl; more preferably, from isopropyl, tert-butyl, cyclohexyl and tetrahydropyranyl, most preferably from cyclohexyl;

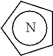

of Formula (XVI) is a 5- or 6-membered nitrogen-containing heteroaryl; more preferably, 5-membered nitrogen-containing heteroaryl, and most preferably thiazole; and
Ar of Formula (XVI) is an aryl or a heteroaryl.

In any of the compounds described herein, the ILM can have the structure of Formula (XVII), which is based on the IAP ligands described in Cohen, F. et al., Antogonists of inhibitors of apoptosis proteins based on thiazole amide isosteres, Bioorg. Med. Chem. Lett., 20(7), 2229-33 (2010), or an unnatural mimetic thereof:

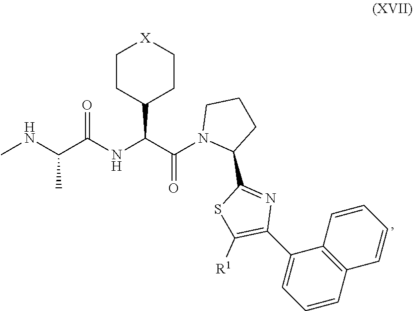

wherein:

R¹ of Formula (XVII) is selected from the group halogen (e.g. fluorine), cyano,

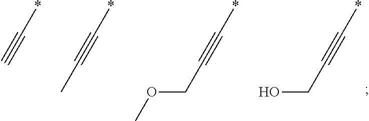

X of Formula (XVII) is selected from the group O or CH₂.

In any of the compounds described herein, the ILM can have the structure of Formula (XVIII), which is based on the IAP ligands described in Cohen, F. et al., Antogonists of inhibitors of apoptosis proteins based on thiazole amide isosteres, Bioorg. Med. Chem. Lett., 20(7), 2229-33 (2010), or an unnatural mimetic thereof:

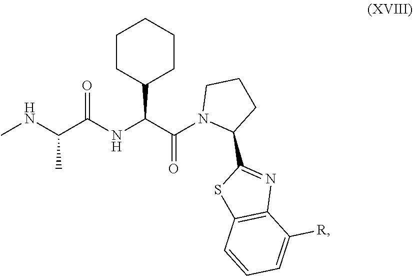

wherein R of Formula (XVIII) is selected from alkyl, aryl, heteroaryl, arylalkyl, heteroarylalkyl or halogen (in variable substitution position).

In any of the compounds described herein, the ILM can have the structure of Formula (XIX), which is based on the IAP ligands described in Cohen, F. et al., Antagonists of inhibitors of apoptosis proteins based on thiazole amide isosteres, Bioorg. Med. Chem. Lett., 20(7), 2229-33 (2010), or an unnatural mimetic thereof:

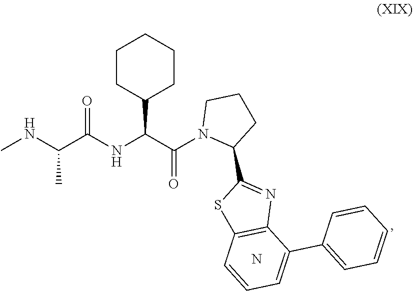

wherein

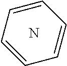

is a 6-member nitrogen heteroaryl.

In a certain embodiment, the ILM of the composition is selected from the group consisting of:

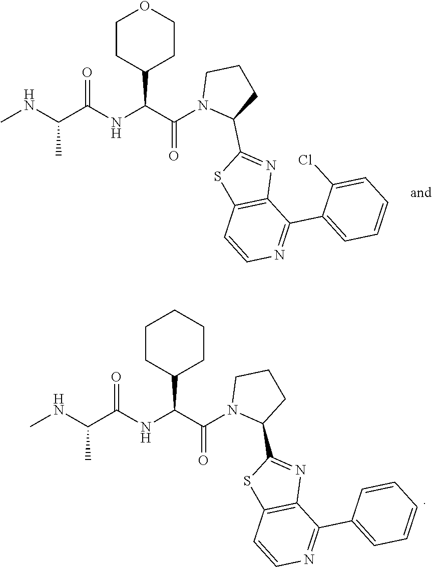 [SMILES: CN[C@@H](C)C(=O)N[C@H](C(=O)N1CCC[C@H]1c1nc2c(-c3ccccc3Cl)nccc2s1)C1CCOCC1 ; CN[C@@H](C)C(=O)N[C@H](C(=O)N1CCC[C@H]1c1nc2c(-c3ccccc3)nccc2s1)C1CCCCC1]

In certain embodiments, the ILM of the composition is selected from the group consisting of:

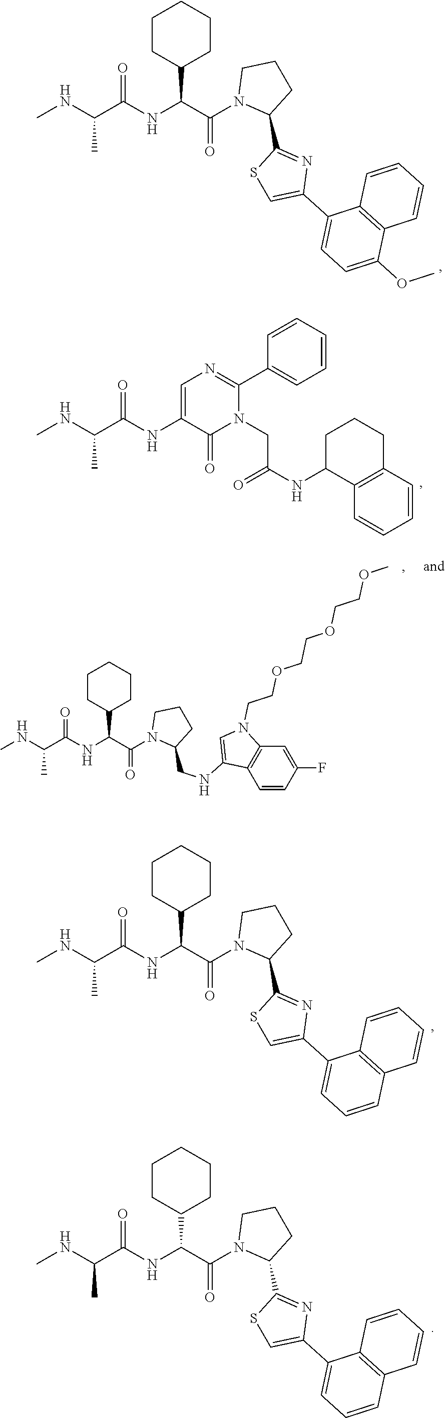

In any of the compounds described herein, the ILM can have the structure of Formula (XX), which is based on the IAP ligands described in WO Pub. No. 2007/101347, or an unnatural mimetic thereof:

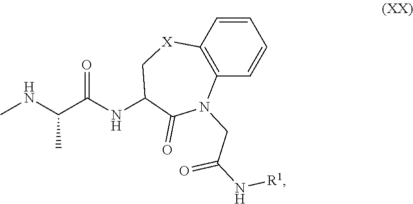

wherein X of Formula (XX) is selected from CH₂, O, NH, or S.

In any of the compounds described herein, the ILM can have the structure of Formula (XXI), which is based on the IAP ligands described in U.S. Pat. Nos. 7,345,081 and 7,419,975. or an unnatural mimetic thereof:

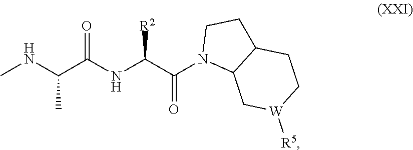

wherein:
R² of Formula (XXI) is selected from:

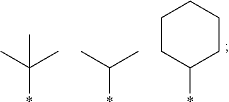

R⁵ of Formula (XXI) is selected from:

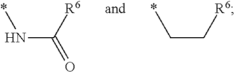

and
W of Formula (XXI) is selected from CH or N; and
R⁶ of

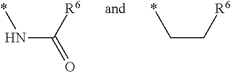

are independently a mono- or bicyclic fused aryl or heteroaryl.

In certain embodiments, the ILM of the compound is selected from the group consisting of:

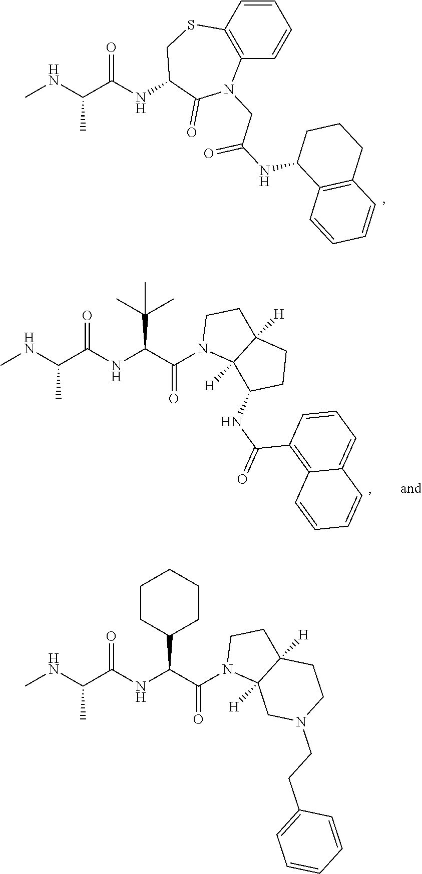 [SMILES: CN[C@@H](C)C(=O)N[C@@H]1CSc2ccccc2N(CC(=O)N[C@@H]2CCCc3ccccc32)C1=O ; CN[C@@H](C)C(=O)N[C@H](C(=O)N1CC[C@H]2CC[C@H](NC(=O)c3cccc4ccccc34)[C@H]21)C(C)(C)C ; CN[C@@H](C)C(=O)N[C@H](C(=O)N1CC[C@H]2CCN(CCc3ccccc3)C[C@H]21)C1CCCCC1]

In any of the compounds described herein, the ILM can have the structure of Formula (XXII) or (XXIV), which are derived from the IAP ligands described in WO Pub. No. 2015/006524 and Perez H L, Discovery of potent heterodimeric antagonists of inhibitor of apoptosis proteins (IAPs) with sustained antitumor activity. J. Med. Chem. 58(3), 1556-62 (2015), or an unnatural mimetic thereof, and the chemical linker to linker group L as shown:

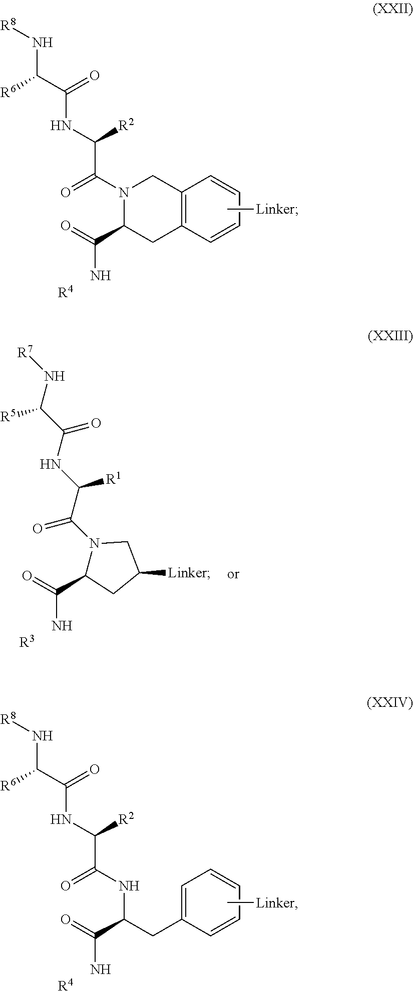

wherein:
R¹ of Formula (XXII), (XXIII) or (XXIV) is selected from optionally substituted alkyl, optionally substituted cycloalkyl, optionally substituted cycloalkylalkyl, optionally substituted heterocyclyl, optionally substituted arylalkyl or optionally substituted aryl;
R² of Formula (XXII), (XXIII) or (XXIV) is selected from optionally substituted alkyl, optionally substituted cycloalkyl, optionally substituted cycloalkylalkyl, optionally substituted heterocyclyl, optionally substituted arylalkyl or optionally substituted aryl;
or alternatively,
R¹ and R² of Formula (XXII), (XXIII) or (XXIV) are independently selected from optionally substituted thioalkyl wherein the substituents attached to the S atom of the thioalkyl are optionally substituted alkyl, optionally substituted branched alkyl, optionally substituted heterocyclyl, —(CH₂)ᵥCOR²⁰, —CH₂CHR²¹COR²² or —CH₂R²³,
wherein:
   v is an integer from 1-3;
   R²⁰ and R²² of —(CH₂)ᵥCOR²⁰ and —CH₂R²³ are independently selected from OH, NR²⁴R²⁵ or OR²⁶;
   R²¹ of —CH₂CHR²¹COR² is selected from NR²⁴R²⁵;
   R²³ of —CH₂R²³ is selected from optionally substituted aryl or optionally substituted heterocyclyl, wherein the optional substituents include alkyl and halogen;
   R²⁴ of NR²⁴R²⁵ is selected from hydrogen or optionally substituted alkyl;
   R²⁵ of NR²⁴R²⁵ is selected from hydrogen, optionally substituted alkyl, optionally substituted branched alkyl, optionally substituted arylalkyl, optionally substituted heterocyclyl, —CH₂(OCH₂CH₂O)ₘCH₃, or a polyamine chain, such as spermine or spermidine;
   R²⁶ of OR²⁶ is selected from optionally substituted alkyl, wherein the optional substituents are OH, halogen or NH₂; and
   m is an integer from 1-8;
   R³ and R⁴ of Formula (XXII), (XXIII) or (XXIV) are independently optionally substituted alkyl, optionally substituted cycloalkyl, optionally substituted aryl, optionally substituted arylalkyl, optionally substituted arylalkoxy, optionally substituted heteroaryl, optionally substituted heterocyclyl, optionally substituted heteroarylalkyl or optionally substituted heterocycloalkyl, wherein the substituents are alkyl, halogen or OH;
   R⁵, R⁶, R⁷ and R⁸ of Formula (XXII), (XXIII) or (XXIV) are independently hydrogen, optionally substituted alkyl or optionally substituted cycloalkyl; and/or a pharmaceutically acceptable salt, tautomer or stereoisomer thereof.

In a particular embodiment, the ILM according to Formulas (XXII) through (XXIV):

R⁷ and R⁸ are selected from the H or Me;
R⁵ and R⁶ are selected from the group comprising:

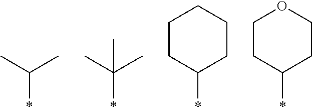

R³ and R⁴ are selected from the group comprising:

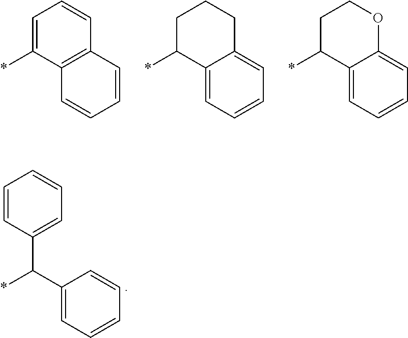

In any of the compounds described herein, the ILM can have the structure of Formula (XXV), (XXVI), (XXVII), or (XXVIII), which are derived from the IAP ligands described in WO Pub. No. 2014/055461 and Kim, K S, Discovery of tetrahydroisoquinoline-based bivalent heterodimeric IAP antagonists. Biooig. Med. Chem. Lett. 24(21), 5022-9 (2014), or an unnatural mimetic thereof, and the chemical linker to linker group L as shown:

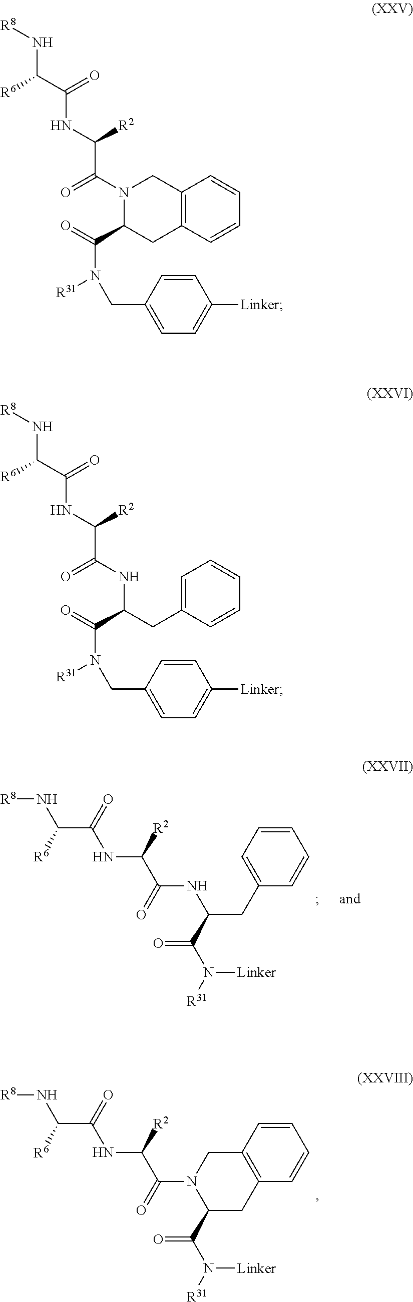

wherein:
R² of Formula (XXV) through (XXVIII) is selected from H, an optionally substituted alkyl, optionally substituted cycloalkyl, optionally substituted cycloalkylalkyl, optionally substituted heterocyclyl, optionally substituted arylalkyl or optionally substituted aryl;
or alternatively;
R¹ and R² of Formula (XXV) and (XXVIII) are independently selected from H, an optionally substituted thioalkyl —CR⁶⁰R⁶¹SR⁷⁰ wherein R⁶⁰ and R⁶¹ are selected from H or methyl, and R⁷⁰ is an optionally substituted alkyl, optionally substituted branched alkyl, optionally substituted heterocyclyl, —(CH₂)ᵥCOR²⁰, —CH₂CHR²¹COR²² or —CH₂R²³;
wherein:
v is an integer from 1-3;
R²⁰ and R²² of —(CH₂)ᵥCOR²⁰ and —CH₂CHR²¹COR²² are independently selected from OH, NR²⁴R²⁵ or OR²⁶;
R²¹ of —CH₂CHR²¹COR²² is selected from NR²⁴R²⁵;
R²³ of —CH₂R²³ is selected from an optionally substituted aryl or optionally substituted heterocyclyl, where the optional substituents include alkyl and halogen;
R²⁴ of NR²⁴R²⁵ is selected from hydrogen or optionally substituted alkyl;
R²⁵ of NR²⁴R²⁵ is selected from hydrogen, optionally substituted alkyl, optionally substituted branched alkyl, optionally substituted arylalkyl, optionally substituted heterocyclyl, —CH₂CH₂(OCH₂CH₂)ₘCH₃, or a polyamine chain —[CH₂CH₂(CH₂)_{δ}NH]_{ψ}CH₂CH₂(CH₂)ω̅ᵣNH₂, such as spermine or spermidine,
wherein δ=0-2, ψ=1-3, ω̅=0-2;
R²⁶ of OR²⁶ is an optionally substituted alkyl, wherein the optional substituents are OH, halogen or NH₂;
m is an integer from 1-8;
R⁶ and R⁸ of Formula (XXV) through (XXVIII) are independently selected from hydrogen, optionally substituted alkyl or optionally substituted cycloalkyl; and
R³¹ of Formulas (XXV) through (XXVIII) is selected from alkyl, aryl, arylalkyl, heteroaryl or heteroarylalkyl optionally further substituted, preferably selected form the group consisting of;

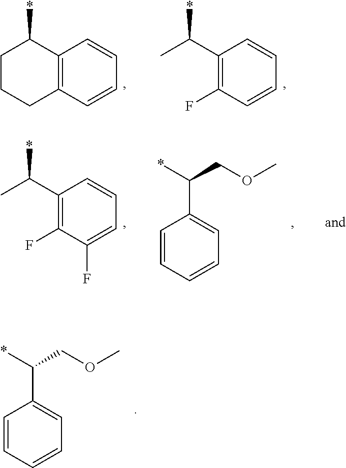

In any of the compounds described herein, the ILM can have the structure of Formula (XXIX) or (XXX), which are derived from the IAP ligands described in WO Pub. No. 2013/071039, or an unnatural mimetic thereof:

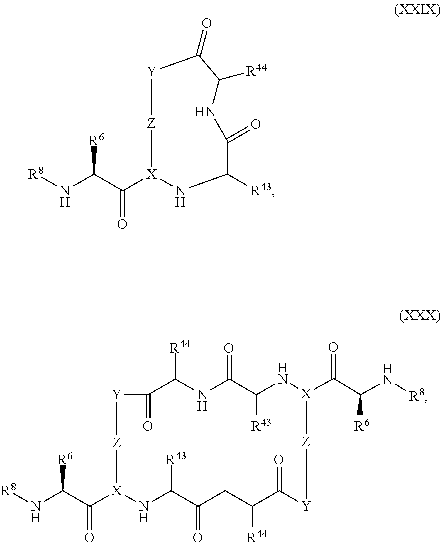

wherein:
R⁴³ and R⁴⁴ of Formulas (XXIX) and (XXX) are independently selected from hydrogen, alkyl, aryl, arylalkyl, heteroaryl, heteroarylalkyl, cycloalkyl, cycloalkylalkyl further optionally substituted, and
R⁶ and R⁸ of Formula (XXIX) and (XXX) are independently selected from hydrogen, optionally substituted alkyl or optionally substituted cycloalkyl.
each X of Formulas (XXIX) and (XXX) is independently selected from:

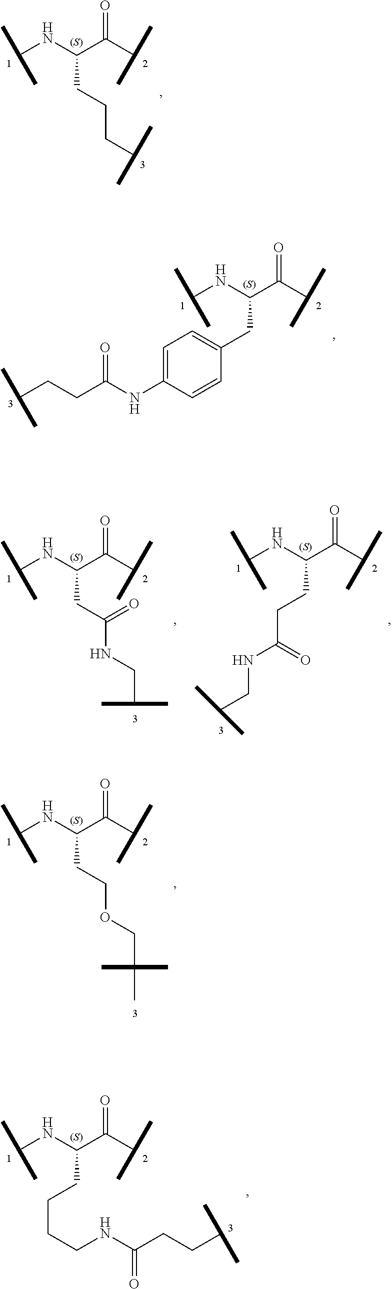

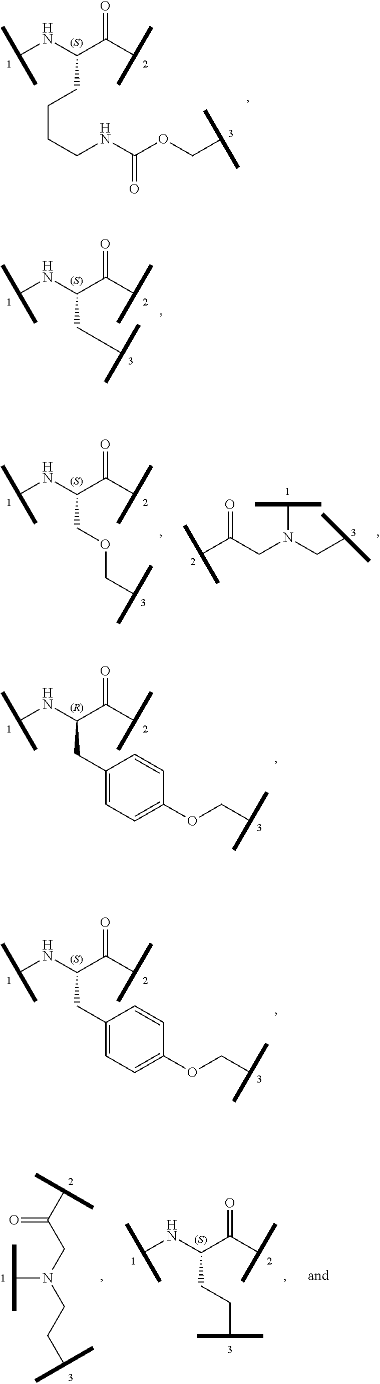

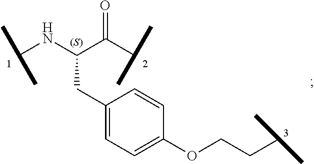

each Z of Formulas (XXIX) and (XXX) is selected from

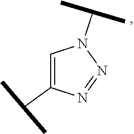

wherein each

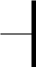

represents a point of attachment to the compound; and
each Y is selected from:

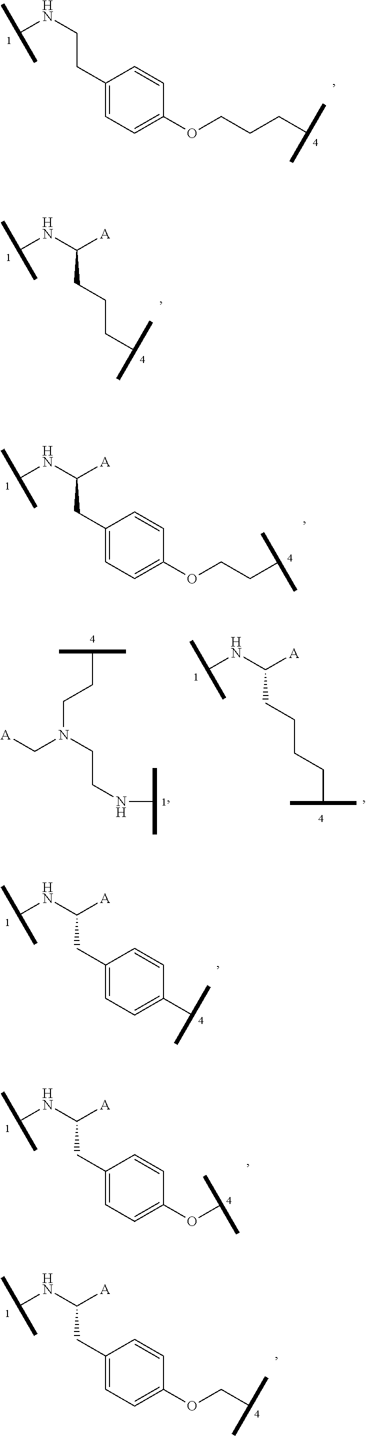

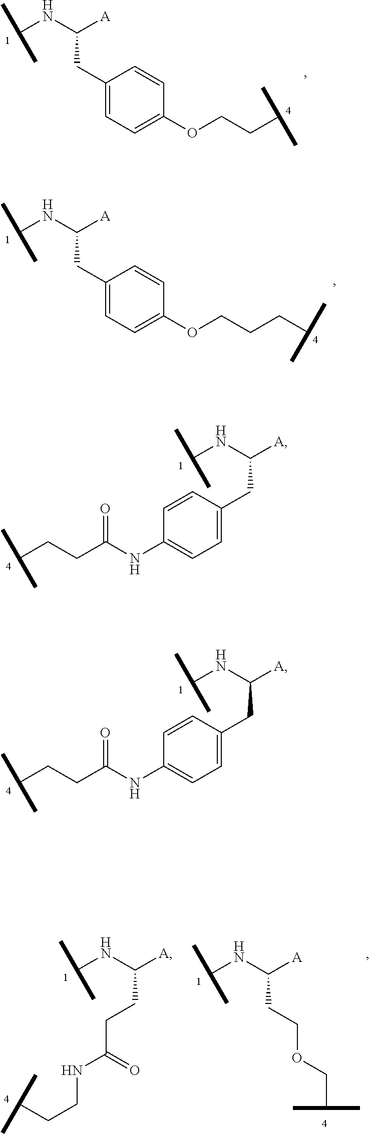

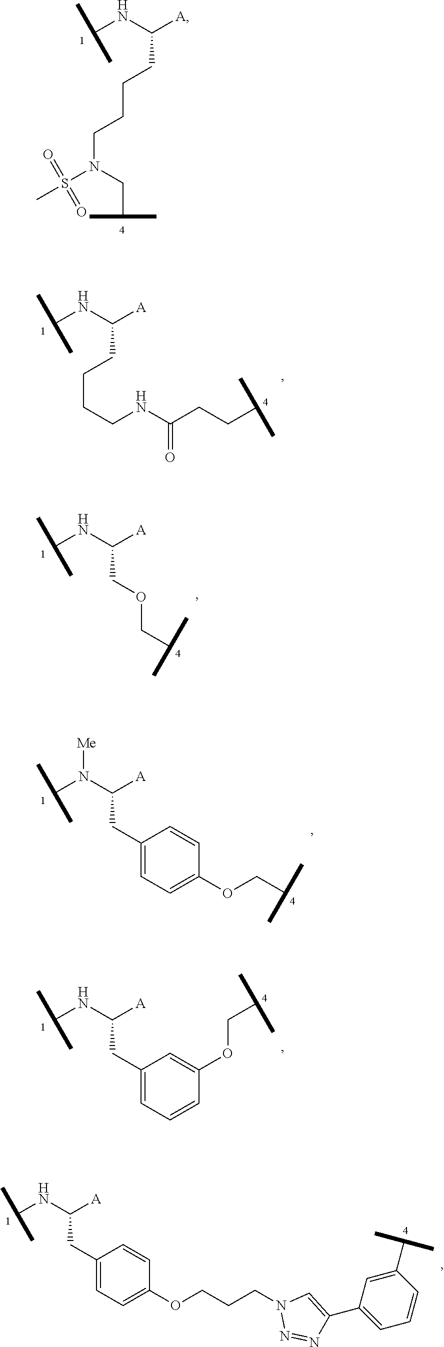

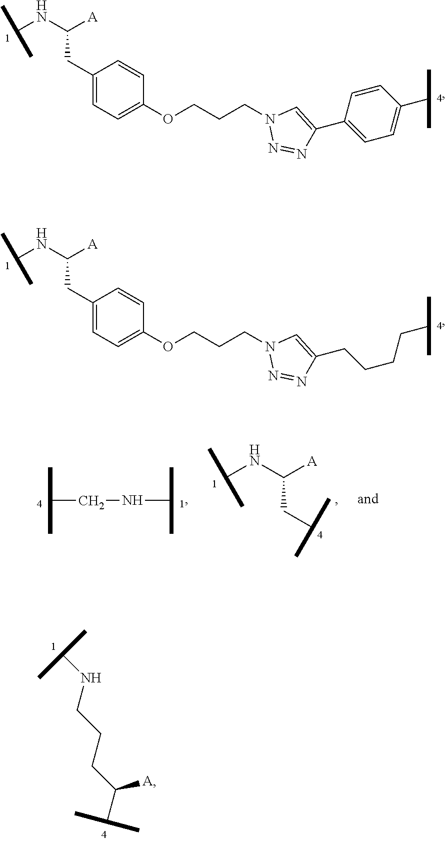

wherein:

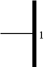

represents a point of attachment to a —C═O portion of the compound;

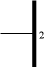

represents a point of attachment to an amino portion of the compound;

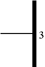

represents a first point of attachment to Z;

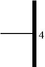

represents a second point of attachment to Z; and
A is selected from —C(O)R³ or

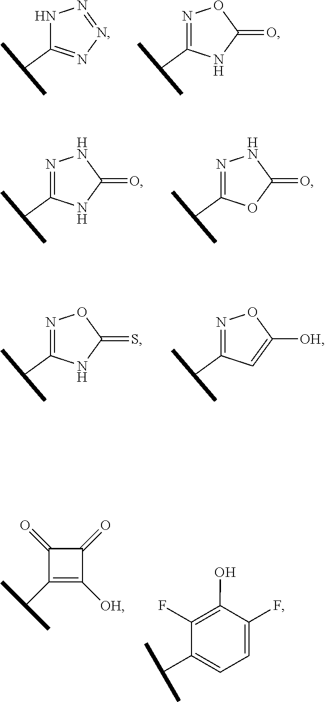

or a tautomeric form of any of the foregoing, wherein:
R³ of —C(O)R³ is selected from OH, NHCN, NHS0₂R¹⁰, NHOR¹¹ or N(R¹²)(R¹³);
R¹⁰ and R¹¹ of NHS0₂R¹⁰ and NHOR¹¹ are independently selected from —C₁-C₄ alkyl, cycloalkyl, aryl, heteroaryl, or heterocycloalkyl, any of which are optionally substituted, and hydrogen;
each of R¹² and R¹³ of N(R¹²)(R¹³) are independently selected from hydrogen, —C₁-C₄ alkyl, —(C₁-C₄ alkylene)-NH—(C₁-C₄ alkyl), benzyl, —(C₁-C₄ alkylene)-C(O)OH,
—(C₁-C₄ alkylene)-C(O)CH₃, —CH(benzyl)-COOH, —C₁-C₄ alkoxy, and
—(C₁-C₄ alkylene)-O—(C₁-C₄ hydroxyalkyl); or R¹² and R¹³ of N(R¹²)(R¹³) are taken together with the nitrogen atom to which they are commonly bound to form a saturated heterocyclyl optionally comprising one additional heteroatom selected from N, O and S, and wherein the saturated heterocycle is optionally substituted with methyl.

In any of the compounds described herein, the ILM can have the structure of Formula (XXXI), which are derived from the IAP ligands described in WO Pub. No. 2013/071039, or an unnatural mimetic thereof:

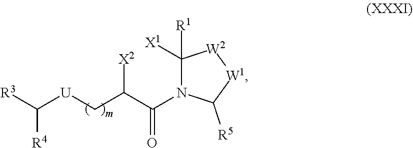

wherein:
W¹ of Formula (XXXI) is selected from O, S, N—R^{A}, or C(R^{8a})(R^{8b});
W² of Formula (XXXI) is selected from O, S, N—R^{A}, or C(R^{8c})(R^{8d}); provided that W¹ and W² are not both O, or both S;
R¹ of Formula (XXXI) is selected from H, C₁-C₆alkyl, C₃-C₆cycloalkyl, —C₁-C₆alkyl-(substituted or unsubstituted C₃-C₆cycloalkyl), substituted or unsubstituted aryl, substituted or unsubstituted heteroaryl, —C₁-C₆alkyl-(substituted or unsubstituted aryl), or —C₁-C₆alkyl-(substituted or unsubstituted heteroaryl);
when X¹ is selected from O, N—R^{A}, S, S(O), or S(O)₂, then X² is C(R^{2a}R^{2b});
or:
X¹ of Formula (XXXI) is selected from CR^{2c}R^{2d} and X² is CR^{2a}R^{2b}, and R^{2c} and R^{2a} together form a bond;
or:
X¹ and X² of Formula (XXXI) are independently selected from C and N, and are members of a fused substituted or unsubstituted saturated or partially saturated 3-10 membered cycloalkyl ring, a fused substituted or unsubstituted saturated or partially saturated 3-10 membered heterocycloalkyl ring, a fused substituted or unsubstituted 5-10 membered aryl ring, or a fused substituted or unsubstituted 5-10 membered heteroaryl ring;
or:
X¹ of Formula (XXXI) is selected from CH₂ and X² is C═O, C═C(R^{C})₂, or C═NR^{C}; where each R^{c} is independently selected from H, —CN, —OH, alkoxy, substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), or —C₁-C₆alkyl-(substituted or unsubstituted heteroaryl);
R^{A} of N—R^{A} is selected from H, C₁-C₆alkyl, —C(═O)C₁-C₂alkyl, substituted or unsubstituted aryl, or substituted or unsubstituted heteroaryl;
R^{2a}, R^{2b}, R^{2c}, R^{2d} of CR^{2c}R^{2d} and CR^{2a}R^{2b} are independently selected from H, substituted or unsubstituted C₁-C₆alkyl, substituted or unsubstituted C₁-C₆heteroalkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), —C₁-C₆alkyl-(substituted or unsubstituted heteroaryl) and —C(═O)R^{B};
R^{B} of —C(═O)R^{B} is selected from substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), —C₁-C₆alkyl-(substituted or unsubstituted heteroaryl), or —NR^{D}R^{E};
R^{D} and R^{E} of NR^{D}R^{E} are independently selected from H, substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), or —C₁-C₆alkyl-(substituted or unsubstituted heteroaryl);
m of Formula (XXXI) is selected from 0, 1 or 2;
—U— of Formula (XXXI) is selected from —NHC(═O)—, —C(═O)NH—, —NHS(═O)₂—, —S(═O)₂NH—, —NHC(═O)NH—, —NH(C═O)O—, —O(C═O)NH—, or —NHS(═O)₂NH—;
R³ of Formula (XLI) is selected from C₁-C₃alkyl, or C₁-C₃fluoroalkyl;
R⁴ of Formula (XLI) is selected from —NHR⁵, —N(R⁵)2, —N+(R⁵)3 or —OR⁵;
each R⁵ of —NHR⁵, —N(R⁵)2, −N+(R⁵)3 and —OR⁵ is independently selected from H, C₁-C₃alkyl, C₁-C₃haloalkyl, C₁-C₃heteroalkyl and —C₁-C₃alkyl-(C₃-C₅cycloalkyl);
or:
R³ and R⁵ of Formula (XXXI) together with the atoms to which they are attached form a substituted or unsubstituted 5-7 membered ring;
or:
R³ of Formula (XXXI) is bonded to a nitrogen atom of U to form a substituted or unsubstituted 5-7 membered ring;
R⁶ of Formula (XXXI) is selected from —NHC(═O)R⁷, —C(═O)NHR⁷, —NHS(═O)2R⁷, —S(═O)₂NHR⁷; —NHC(═O)NHR⁷, —NHS(═O)₂NHR⁷, —(C₁-C₃alkyl)-NHC(═O)R⁷, —(C₁-C₃alkyl)-C(═O)NHR⁷, —(C₁-C₃alkyl)-NHS(═O)2R⁷, —(C₁-C₃alkyl)-S(═O)2NHR⁷; —(C₁-C₃alkyl)-NHC(═O)NHR⁷, —(C₁-C₃alkyl)-NHS(═O)2NHR⁷, substituted or unsubstituted C₂-C₁₀heterocycloalkyl, or substituted or unsubstituted heteroaryl;
each R⁷ of —NHC(═O)R⁷, —C(═O)NHR⁷, —NHS(═O)2R⁷, —S(═O)₂NHR⁷; —NHC(═O)NHR⁷, —NHS(═O)₂NHR⁷, —(C₁-C₃alkyl)-NHC(═O)R⁷, —(C₁-C₃alkyl)-C(═O)NHR⁷, —(C₁-C₃alkyl)-NHS(═O)2R⁷, —(C₁-C₃alkyl)-S(═O)2NHR⁷; —(C₁-C₃alkyl)-NHC(═O)NHR⁷, —(C₁-C₃alkyl)-NHS(═O)2NHR⁷ is independently selected from C₁-C₆alkyl, C₁-C₆haloalkyl, C₁-C₆heteroalkyl, a substituted or unsubstituted C3-C10cycloalkyl, a substituted or unsubstituted C₂-C₁₀heterocycloalkyl, a substituted or unsubstituted aryl, a substituted or unsubstituted heteroaryl, —C₁-C₆alkyl-(substituted or unsubstituted C₃-C₁₀cycloalkyl), —C₁-C₆alkyl-(substituted or unsubstituted C2-C10heterocycloalkyl, —C1-C6alkyl-(substituted or unsubstituted aryl), —C₁-C₆alkyl-(substituted or unsubstituted heteroaryl), —(CH2)p-CH(substituted or unsubstituted aryl)2, —(CH₂)ₚ—CH(substituted or unsubstituted heteroaryl)2, —(CH₂)_{P}—CH(substituted or unsubstituted aryl)(substituted or unsubstituted heteroaryl), -(substituted or unsubstituted aryl)-(substituted or unsubstituted aryl), -(substituted or unsubstituted aryl)-(substituted or unsubstituted heteroaryl), -(substituted or unsubstituted heteroaryl)-(substituted or unsubstituted aryl), or -(substituted or unsubstituted heteroaryl)-(substituted or unsubstituted heteroaryl);
p of R⁷ is selected from 0, 1 or 2;
R^{8a}, R^{8b}, R^{8c}, and R^{8d} of C(R^{8a})(R^{8b}) and C(R^{8c})(R^{8d}) are independently selected from H, C₁-C₆alkyl, C₁-C₆fluoroalkyl, C₁-C₆ alkoxy, C₁-C₆heteroalkyl, and substituted or unsubstituted aryl;
or:
R^{8a} and R^{8d} are as defined above, and R^{8b} and R^{8c} together form a bond;
or:
R^{8a} and R^{8d} are as defined above, and R^{8b} and R^{8c} together with the atoms to which they are attached form a substituted or unsubstituted fused 5-7 membered saturated, or partially saturated carbocyclic ring or heterocyclic ring comprising 1-3 heteroatoms selected from S, O and N, a substituted or unsubstituted fused 5-10 membered aryl ring, or a substituted or unsubstituted fused 5-10 membered heteroaryl ring comprising 1-3 heteroatoms selected from S, O and N;
or:
R^{8a} and R^{8b} are as defined above, and R^{8c} and R^{8d} together with the atoms to which they are attached form a substituted or unsubstituted saturated, or partially saturated 3-7 membered spirocycle or heterospirocycle comprising 1-3 heteroatoms selected from S, O and N;
or:
R^{8a} and R^{8b} are as defined above, and R^{8c} and R^{8d} together with the atoms to which they are attached form a substituted or unsubstituted saturated, or partially saturated 3-7 membered spirocycle or heterospirocycle comprising 1-3 heteroatoms selected from S, O and N;
where each substituted alkyl, heteroalkyl, fused ring, spirocycle, heterospirocycle, cycloalkyl, heterocycloalkyl, aryl or heteroaryl is substituted with 1-3 R⁹; and
each R⁹ of R^{8a}, R^{8b}, R^{8c} and R^{8d} is independently selected from halogen, —OH, —SH, (C═O), CN, C₁-C₄alkyl, C₁-C₄fluoroalkyl, C₁-C₄ alkoxy, C₁-C₄ fluoroalkoxy, —NH₂, —NH(C₁-C₄alkyl), —NH(C₁-C₄alkyl)₂, —C(═O)OH, —C(═O)NH₂, —C(═O)C₁-C₃alkyl, —S(═O)₂CH₃, —NH(C₁-C₄alkyl)-OH, —NH(C₁-C₄alkyl)-O—(C—C₄alkyl), —O(C₁-C₄alkyl)-NH2; —O(C₁-C₄alkyl)-NH—(C₁-C₄alkyl), and —O(C₁-C₄alkyl)-N—(C₁-C₄alkyl)₂, or two R⁹ together with the atoms to which they are attached form a methylene dioxy or ethylene dioxy ring substituted or unsubstituted with halogen, —OH, or C₁-C₃alkyl.

In any of the compounds described herein, the ILM can have the structure of Formula (XXXII), which are derived from the IAP ligands described in WO Pub. No. 2013/071039, or an unnatural mimetic thereof:

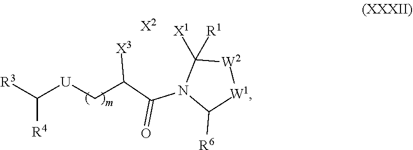

wherein:
W¹ of Formula (XXXII) is O, S, N—R^{A}, or C(R^{8a})(R^{8b});
W² of Formula (XXXH) is O, S, N—R^{A}, or C(R^{8c})(R^{8d}); provided that W¹ and W² are not both O, or both S;
R¹ of Formula (XLII) is selected from H, C₁-C₆alkyl, C₃-C₆cycloalkyl, —C₁-C₆alkyl-(substituted or unsubstituted C₃-C₆cycloalkyl), substituted or unsubstituted aryl, substituted or unsubstituted heteroaryl, —C₁-C₆alkyl-(substituted or unsubstituted aryl), or —C₁-C₆alkyl-(substituted or unsubstituted heteroaryl);
when X¹ of Formula (XXXII) is N—R^{A}, then X² is C═O, or CR^{2c}R^{2d}, and X³ is CR^{2a}R^{2b};
or:
when X¹ of Formula (XXXII) is selected from S, S(O), or S(O)₂, then X² is CR^{2c}R^{2d}, and X³ is CR^{2a}R^{2b};
or:
when X¹ of Formula (XXXII) is O, then X² is CR^{2c}R^{2d} and N—R^{A} and X³ is CR^{2a}R^{2b};
or:
when X¹ of Formula (XXXII) is CH₃, then X² is selected from O, N—R^{A}, S, S(O), or S(O)₂, and X³ is CR^{2a}R^{2b};
when X¹ of Formula (XXXII) is CR^{2e}R^{2f} and X2 is CR^{2c}R^{2d}, and R^{2e} and R^{2c} together form a bond, and X³ of Formula (XXXH) is CR^{2a}R^{2b};
or:
X¹ and X³ of Formula (XXXH) are both CH₂ and X² of Formula (XXXH) is C═O, C═C(R^{C})2, or C═NR^{C}; where each R^{C} is independently selected from H, —CN, —OH, alkoxy, substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), or —C₁-C₆alkyl-(substituted or unsubstituted heteroaryl);
or:
X¹ and X² of Formula (XXXII) are independently selected from C and N, and are members of a fused substituted or unsubstituted saturated or partially saturated 3-10 membered cycloalkyl ring, a fused substituted or unsubstituted saturated or partially saturated 3-10 membered heterocycloalkyl ring, a fused substituted or unsubstituted 5-10 membered aryl ring, or a fused substituted or unsubstituted 5-10 membered heteroaryl ring, and X³ is CR^{2a}R^{2b};
or:
X² and X³ of Formula (XXXII) are independently selected from C and N, and are members of a fused substituted or unsubstituted saturated or partially saturated 3-10 membered cycloalkyl ring, a fused substituted or unsubstituted saturated or partially saturated 3-10 membered heterocycloalkyl ring, a fused substituted or unsubstituted 5-10 membered aryl ring, or a fused substituted or unsubstituted 5-10 membered heteroaryl ring, and X¹ of Formula (XXXII) is CR^{2e}R^{2f};
R^{A} of N—R^{A} is selected from H, C₁-C₆alkyl, —C(═O)C₁-C₂alkyl, substituted or unsubstituted aryl, or substituted or unsubstituted heteroaryl;
R^{2a}, R^{2b}, R^{2c}, R^{2d}, R^{2e}, and R^{2f} of CR^{2c}R^{2d}, CR^{2a}R^{2b} and CR^{2e}R^{2f} are independently selected from H, substituted or unsubstituted C₁-C₆alkyl, substituted or unsubstituted C₁-C₆heteroalkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), —C₁-C₆alkyl-(substituted or unsubstituted heteroaryl) and —C(═O)R^{B};
R^{B} of —C(═O)R^{B} is selected from substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), —C₁-C₆alkyl-(substituted or unsubstituted heteroaryl), or —NR^{D}R^{E};
R^{D} and R^{E} of NR^{D}R^{E} are independently selected from H, substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), or —C₁-C₆alkyl-(substituted or unsubstituted heteroaryl);
m of Formula (XXXII) is selected from 0, 1 or 2;
—U— of Formula (XXXII) is selected from —NHC(═O)—, —C(═O)NH—, —NHS(═O)₂—, —S(═O)₂NH—, —NHC(═O)NH—, —NH(C═O)O—, —O(C═O)NH—, or —NHS(═O)₂NH—;
R³ of Formula (XLII) is selected from C₁-C₃alkyl, or C₁-C₃fluoroalkyl;
R⁴ of Formula (XXXII) is selected from —NHR⁵, —N(R⁵)₂, —N+(R⁵)₃ or —OR⁵;
each R⁵ of —NHR⁵, —N(R⁵)₂, —N+(R⁵)₃ and —OR⁵ is independently selected from H, C₁-C₃alkyl, C₁-C₃haloalkyl, C₁-C₃heteroalkyl and —C₁-C₃alkyl-(C₃-C₅cycloalkyl);
or:
R³ and R⁵ of Formula (XXXII) together with the atoms to which they are attached form a substituted or unsubstituted 5-7 membered ring;
or:
R³ of Formula (XXXII) is bonded to a nitrogen atom of U to form a substituted or unsubstituted 5-7 membered ring;
R⁶ of Formula (XXXII) is selected from —NHC(═O)R⁷, —C(═O)NHR⁷, —NHS(═O)2R⁷, —S(═O)₂NHR⁷; —NHC(═O)NHR⁷, —NHS(═O)₂NHR⁷, —(C₁-C₃alkyl)-NHC(═O)R⁷, —(C₁-C₃alkyl)-C(═O)NHR⁷, —(C₁-C₃alkyl)-NHS(═O)₂R⁷, —(C₁-C₃alkyl)-S(═O)₂NHR⁷; —(C₁-C₃alkyl)-NHC(═O)NHR⁷, —(C₁-C₃alkyl)-NHS(═O)₂NHR⁷, substituted or unsubstituted C₂-C₁₀heterocycloalkyl, or substituted or unsubstituted heteroaryl;
each R⁷ of —NHC(═O)R⁷, —C(═O)NHR⁷, —NHS(═O)2R⁷, —S(═O)₂NHR⁷; —NHC(═O)NHR⁷, —NHS(═O)₂NHR⁷, —(C₁-C₃alkyl)-NHC(═O)R⁷, —(C₁-C₃alkyl)-C(═O)NHR⁷, —(C₁-C₃alkyl)-NHS(═O)2R⁷, —(C₁-C₃alkyl)-S(═O)2NHR⁷; —(C₁-C₃alkyl)-NHC(═O)NHR⁷, —(C₁-C₃alkyl)-NHS(═O)2NHR⁷ is independently selected from C₁-C₆alkyl, C₁-C₆haloalkyl, C₁-C₆heteroalkyl, a substituted or unsubstituted C3-C10cycloalkyl, a substituted or unsubstituted C₂-C₁₀heterocycloalkyl, a substituted or unsubstituted aryl, a substituted or unsubstituted heteroaryl, —C₁-C₆alkyl-(substituted or unsubstituted C₃-C₁₀cycloalkyl), —C₁-C₆alkyl-(substituted or unsubstituted C₂-C₁₀heterocycloalkyl, —C₁-C₆alkyl-(substituted or unsubstituted aryl), —C₁-C₆alkyl-(substituted or unsubstituted heteroaryl), —(CH2)p-CH(substituted or unsubstituted aryl)2, —(CH₂)ₚ—CH(substituted or unsubstituted heteroaryl)₂, —(CH₂)_{P}—CH(substituted or unsubstituted aryl)(substituted or unsubstituted heteroaryl), -(substituted or unsubstituted aryl)-(substituted or unsubstituted aryl), -(substituted or unsubstituted aryl)-(substituted or unsubstituted heteroaryl), -(substituted or unsubstituted heteroaryl)-(substituted or unsubstituted aryl), or -(substituted or unsubstituted heteroaryl)-(substituted or unsubstituted heteroaryl);
p of R⁷ is selected from 0, 1 or 2;
R^{8a}, R^{8b}, R^{8c}, and R^{8d} of C(R^{8a})(R^{8b}) and C(R^{8c})(R^{8d}) are independently selected from H, C₁-C₆alkyl, C₁-C₆fluoroalkyl, C₁-C₆ alkoxy, C₁-C₆heteroalkyl, and substituted or unsubstituted aryl;
or:
R^{8a} and R^{8d} are as defined above, and R^{8b} and R^{8c} together form a bond;
or:
R^{8a} and R^{8d} are as defined above, and R^{8a} and R^{8b} together with the atoms to which they are attached form a substituted or unsubstituted fused 5-7 membered saturated, or partially saturated carbocyclic ring or heterocyclic ring comprising 1-3 heteroatoms selected from S, O and N, a substituted or unsubstituted fused 5-10 membered aryl ring, or a substituted or unsubstituted fused 5-10 membered heteroaryl ring comprising 1-3 heteroatoms selected from S, O and N;
or:
R^{8c} and R^{8d} are as defined above, and R^{8a} and R^{8b} together with the atoms to which they are attached form a substituted or unsubstituted saturated, or partially saturated 3-7 membered spirocycle or heterospirocycle comprising 1-3 heteroatoms selected from S, O and N;
or:
R^{8a} and R^{8b} are as defined above, and R^{8c} and R^{8d} together with the atoms to which they are attached form a substituted or unsubstituted saturated, or partially saturated 3-7 membered spirocycle or heterospirocycle comprising 1-3 heteroatoms selected from S, O and N;
where each substituted alkyl, heteroalkyl, fused ring, spirocycle, heterospirocycle, cycloalkyl, heterocycloalkyl, aryl or heteroaryl is substituted with 1-3 R⁹; and
each R⁹ of R^{8a}, R^{8b}, R^{8c} and R^{8d} is independently selected from halogen, —OH, —SH, (C═O), CN, C₁-C₄alkyl, C1-C4fluoroalkyl, C₁-C₄ alkoxy, C₁-C₄ fluoroalkoxy, —NH₂, —NH(C₁-C₄alkyl), —NH(C₁-C₄alkyl)₂, —C(═O)OH, —C(═O)NH₂, —C(═O)C₁-C₃alkyl, —S(═O)₂CH₃, —NH(C₁-C₄alkyl)-OH, —NH(C₁-C₄alkyl)-O—(C₁-C₄alkyl), —O(C₁-C₄alkyl)-NH2; —O(C₁-C₄alkyl)-NH—(C₁-C₄alkyl), and —O(C₁-C₄alkyl)-N—(C₁-C₄alkyl)₂, or two R⁹ together with the atoms to which they are attached form a methylene dioxy or ethylene dioxy ring substituted or unsubstituted with halogen, —OH, or C₁-C₃alkyl.

In any of the compounds described herein, the ILM can have the structure of Formula (XXXIII), which is derived from the IAP ligands described in WO Pub. No. 2013/071039, or an unnatural mimetic thereof:

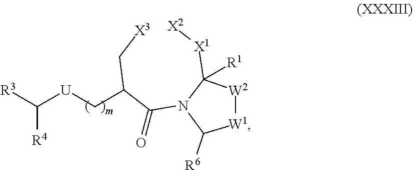

wherein:
W¹ of Formula (XXXIII) is selected from O, S, N—R^{A}, or C(R^{8a})(R^{8b});
W² of Formula (XXXIII) is selected from O, S, N—R^{A}, or C(R^{8c})(R^{8d}); provided that W¹ and W² are not both O, or both S;
R¹ of Formula (XXXIII) is selected from H, C₁-C₆alkyl, C₃-C₆cycloalkyl, —C₁-C₆alkyl-(substituted or unsubstituted C₃-C₆cycloalkyl), substituted or unsubstituted aryl, substituted or unsubstituted heteroaryl, —C₁-C₆alkyl-(substituted or unsubstituted aryl), or —C₁-C₆alkyl-(substituted or unsubstituted heteroaryl);
when X¹ of Formula (XXXIII) is selected from N—R^{A}, S, S(O), or S(O)₂, then X² of Formula (XXXIII) is CR^{2c}R^{2d}, and X³ of Formula (XXXIII) is CR^{2a}R^{2b};
or:
when X¹ of Formula (XXXIII) is O, then X² of Formula (XXXIII) is selected from O, N—R^{A}, S, S(O), or S(O)₂, and X³ of Formula (XXXIII) is CR^{2a}R^{2b};
or:
when X¹ of Formula (XXXIII) is CR^{2e}R^{2f} and X² of Formula (XXXIII) is CR^{2c}R^{2d}, and R^{2e} and R^{2c} together form a bond, and X³ of Formula (XXXIII) is CR^{2a}R^{2b};
or:
X¹ and X² of Formula (XXXIII) are independently selected from C and N, and are members of a fused substituted or unsubstituted saturated or partially saturated 3-10 membered cycloalkyl ring, a fused substituted or unsubstituted saturated or partially saturated 3-10 membered heterocycloalkyl ring, a fused substituted or unsubstituted 5-10 membered aryl ring, or a fused substituted or unsubstituted 5-10 membered heteroaryl ring, and X³ of Formula (XXXIII) is CR^{2a}R^{2b};
or:
X² and X³ of Formula (XXXIII) are independently selected from C and N, and are members of a fused substituted or unsubstituted saturated or partially saturated 3-10 membered cycloalkyl ring, a fused substituted or unsubstituted saturated or partially saturated 3-10 membered heterocycloalkyl ring, a fused substituted or unsubstituted 5-10 membered aryl ring, or a fused substituted or unsubstituted 5-10 membered heteroaryl ring, and X¹ of Formula (XXXIII) is CR^{2e}R^{2f};
R^{A} of N—R^{A} is H, C₁-C₆alkyl, —C(═O)C₁-C₂alkyl, substituted or unsubstituted aryl, or substituted or unsubstituted heteroaryl;
R^{2a}, R^{2b}, R^{2c}, R^{2d}, R^{2e}, and R^{2f} of CR^{2c}R^{2d}, CR^{2a}R^{2b} and CR^{2e}R^{2f} are independently selected from H, substituted or unsubstituted C₁-C₆alkyl, substituted or unsubstituted C₁-C₆heteroalkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), —C₁-C₆alkyl-(substituted or unsubstituted heteroaryl) and —C(═O)R^{B};
R^{B} of —C(═O)R^{B} is substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂—C₅heterocycloalkyl), —C₁-C₆alkyl-(substituted or unsubstituted aryl), —C₁-C₆alkyl-(substituted or unsubstituted heteroaryl), or —NR^{D}R^{E};
R^{D} and R^{E} of NR^{D}R^{E} are independently selected from H, substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), or —C₁-C₆alkyl-(substituted or unsubstituted heteroaryl);
m of Formula (XXXIII) is 0, 1 or 2;
—U— of Formula (XXXIII) is —NHC(═O)—, —C(═O)NH—, —NHS(═O)₂—, —S(═O)₂NH—, —NHC(═O)NH—, —NH(C═O)O—, —O(C═O)NH—, or —NHS(═O)₂NH—;
R³ of Formula (XXXIII) is C₁-C₃alkyl, or C₁-C₃fluoroalkyl;
R⁴ of Formula (XXXIII) is —NHR⁵, —N(R⁵)₂, —N+(R⁵)₃ or —OR⁵;
each R⁵ of —NHR⁵, —N(R⁵)₂, —N+(R⁵)₃ and —OR⁵ is independently selected from H, C₁-C₃alkyl, C₁-C₃haloalkyl, C₁-C₃heteroalkyl and —C₁-C₃alkyl-(C₃-C₅cycloalkyl);
or:
R³ and R⁵ of Formula (XXXIII) together with the atoms to which they are attached form a substituted or unsubstituted 5-7 membered ring;
or:
R³ of Formula (XXXIII) is bonded to a nitrogen atom of U to form a substituted or unsubstituted 5-7 membered ring;
R⁶ of Formula (XXXIII) is selected from —NHC(═O)R⁷, —C(═O)NHR⁷, —NHS(═O)2R⁷, —S(═O)₂NHR⁷; —NHC(═O)NHR⁷, —NHS(═O)₂NHR⁷, —(C₁-C₃alkyl)-NHC(═O)R⁷, —(C₁-C₃alkyl)-C(═O)NHR⁷, —(C₁-C₃alkyl)-NHS(═O)₂R⁷, —(C₁-C₃alkyl)-S(═O)₂NHR⁷; —(C₁-C₃alkyl)-NHC(═O)NHR⁷, —(C₁-C₃alkyl)-NHS(═O)₂NHR⁷, substituted or unsubstituted C₂-C₁₀heterocycloalkyl, or substituted or unsubstituted heteroaryl;
each R⁷ of —NHC(═O)R⁷, —C(═O)NHR⁷, —NHS(═O)2R⁷, —S(═O)₂NHR⁷; —NHC(═O)NHR⁷, —NHS(═O)₂NHR⁷, —(C₁-C₃alkyl)-NHC(═O)R⁷, —(C₁-C₃alkyl)-C(═O)NHR⁷, —(C₁-C₃alkyl)-NHS(═O)2R⁷, —(C₁-C₃alkyl)-S(═O)2NHR⁷; —(C₁-C₃alkyl)-NHC(═O)NHR⁷, —(C₁-C₃alkyl)-NHS(═O)2NHR⁷ is independently selected from C₁-C₆alkyl, C₁-C₆haloalkyl, C₁-C₆heteroalkyl, a substituted or unsubstituted C₃-C₁₀cycloalkyl, a substituted or unsubstituted C₂-C₁₀heterocycloalkyl, a substituted or unsubstituted aryl, a substituted or unsubstituted heteroaryl, —C₁-C₆alkyl-(substituted or unsubstituted C₃-C₁₀cycloalkyl), —C₁-C₆alkyl-(substituted or unsubstituted C₂-C₁₀heterocycloalkyl, —C₁-C₆alkyl-(substituted or unsubstituted aryl), —C₁-C₆alkyl-(substituted or unsubstituted heteroaryl), —(CH₂)ₚ—CH(substituted or unsubstituted aryl)2, —(CH₂)ₚ—CH(substituted or unsubstituted heteroaryl)2, —(CH₂)ₚ—CH(substituted or unsubstituted aryl)(substituted or unsubstituted heteroaryl), -(substituted or unsubstituted aryl)-(substituted or unsubstituted aryl), -(substituted or unsubstituted aryl)-(substituted or unsubstituted heteroaryl), -(substituted or unsubstituted heteroaryl)-(substituted or unsubstituted aryl), or -(substituted or unsubstituted heteroaryl)-(substituted or unsubstituted heteroaryl);
p of R⁷ is 0, 1 or 2;
R^{8a}, R^{8b}, R^{8c}, and R^{8d} of C(R^{8a})(R^{8b}) and C(R^{8c})(R^{8d}) are independently selected from H, C₁-C₆alkyl, C₁-C₆fluoroalkyl, C₁-C₆ alkoxy, C₁-C₆heteroalkyl, and substituted or unsubstituted aryl;
or:
R^{8a} and R^{8d} are as defined above, and R^{8b} and R^{8c} together form a bond;
or:
R^{8a} and R^{8d} are as defined above, and R^{8b} and R^{8c} together with the atoms to which they are attached form a substituted or unsubstituted fused 5-7 membered saturated, or partially saturated carbocyclic ring or heterocyclic ring comprising 1-3 heteroatoms selected from S, O and N, a substituted or unsubstituted fused 5-10 membered aryl ring, or a substituted or unsubstituted fused 5-10 membered heteroaryl ring comprising 1-3 heteroatoms selected from S, O and N;
or:
R^{8c} and R^{8d} are as defined above, and R^{8a} and R^{8b} together with the atoms to which they are attached form a substituted or unsubstituted saturated, or partially saturated 3-7 membered spirocycle or heterospirocycle comprising 1-3 heteroatoms selected from S, O and N;
or:
R^{8a} and R^{8b} are as defined above, and R^{8c} and R^{8d} together with the atoms to which they are attached form a substituted or unsubstituted saturated, or partially saturated 3-7 membered spirocycle or heterospirocycle comprising 1-3 heteroatoms selected from S, O and N;
where each substituted alkyl, heteroalkyl, fused ring, spirocycle, heterospirocycle, cycloalkyl, heterocycloalkyl, aryl or heteroaryl is substituted with 1-3 R⁹; and
each R⁹ of R^{8a}, R^{8b}, R^{8c} and R^{8d} is independently selected from halogen, —OH, —SH, (C═O), CN, C₁-C₄alkyl, C1-C4fluoroalkyl, C₁-C₄ alkoxy, C₁-C₄ fluoroalkoxy, —NH₂, —NH(C₁-C₄alkyl), —NH(C₁-C₄alkyl)₂, —C(═O)OH, —C(=0)NH₂, —C(═O)C₁-C₃alkyl, —S(═O)₂CH₃, —NH(C₁-C₄alkyl)-OH, —NH(C₁-C₄alkyl)-O—(C₁-C₄alkyl), —O(C₁-C₄alkyl)-NH2; —O(C₁-C₄alkyl)-NH—(C₁-C₄alkyl), and —O(C₁-C₄alkyl)-N—(C₁-C₄alkyl)₂, or two R⁹ together with the atoms to which they are attached form a methylene dioxy or ethylene dioxy ring substituted or unsubstituted with halogen, —OH, or C₁-C₃alkyl.

In any of the compounds described herein, the ILM can have the structure of Formula (XXXIV), which is derived from the IAP ligands described in WO Pub. No. 2013/071039, or an unnatural mimetic thereof:

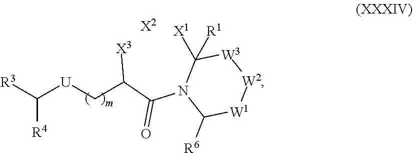

wherein:
W¹ of Formula (XXXIV) is selected from O, S, N—R^{A}, or C(R^{8a})(R^{8b});
W² of Formula (XXXIV) is selected from O, S, N—R^{A}, or C(R^{8c})(R^{8d}); provided that W¹ and W² are not both O, or both S;
W³ of Formula (XXXIV) is selected from O, S, N—R^{A}, or C(R^{8e})(R^{8f}), providing that the ring comprising W¹, W², and W³ does not comprise two adjacent oxygen atoms or sulfur atoms;
R¹ of Formula (XXXIV) is selected from H, C₁-C₆alkyl, C₃-C₆cycloalkyl, —C₁-C₆alkyl-(substituted or unsubstituted C₃-C₆cycloalkyl), substituted or unsubstituted aryl, substituted or unsubstituted heteroaryl, —C₁-C₆alkyl-(substituted or unsubstituted aryl), or —C₁-C₆alkyl-(substituted or unsubstituted heteroaryl);
when X¹ of Formula (XXXIV) is O, then X² of Formula (XXXIV) is selected from CR^{2c}R^{2d} and N—R^{A}, and X³ of Formula (XLIV) is CR^{2a}R^{2b};
or:
when X¹ of Formula (XXXIV) is CH₂, then X² of Formula (XXXIV) is selected from O, N—R^{A}, S, S(O), or S(O)₂, and X³ of Formula (XXXIV) is CR^{2a}R^{2b};
or:
when X¹ of Formula (XXXIV) is CR^{2e}R^{2f} and X² of Formula (XXXIV) is CR^{2c}R^{2d}, and R^{2e} and R^{2c} together form a bond, and X³ of Formula (VLIV) is CR^{2a}R^{2b};
or:
X¹ and X³ of Formula (XXXIV) are both CH₂ and X² of Formula (XXXIV) is C═O, C═C(R^{C})2, or C═NR^{C}; where each R^{C} is independently selected from H, —CN, —OH, alkoxy, substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), or —C₁-C₆alkyl-(substituted or unsubstituted heteroaryl);
or:
X¹ and X² of Formula (XXXIV) are independently selected from C and N, and are members of a fused substituted or unsubstituted saturated or partially saturated 3-10 membered cycloalkyl ring, a fused substituted or unsubstituted saturated or partially saturated 3-10 membered heterocycloalkyl ring, a fused substituted or unsubstituted 5-10 membered aryl ring, or a fused substituted or unsubstituted 5-10 membered heteroaryl ring, and X³ of Formula (XXXIV) is CR^{2a}R^{2b};
or:
X² and X³ of Formula (XXXIV) are independently selected from C and N, and are members of a fused substituted or unsubstituted saturated or partially saturated 3-10 membered cycloalkyl ring, a fused substituted or unsubstituted saturated or partially saturated 3-10 membered heterocycloalkyl ring, a fused substituted or unsubstituted 5-10 membered aryl ring, or a fused substituted or unsubstituted 5-10 membered heteroaryl ring, and X¹ of Formula (XXXIV) is CR^{2e}R^{2f};
R^{A} of N—R^{A} is selected from H, C₁-C₆alkyl, —C(═O)C₁-C₂alkyl, substituted or unsubstituted aryl, or substituted or unsubstituted heteroaryl;
R^{2a}, R^{2b}, R^{2c}, R^{2d}, R^{2e}, and R^{2f} of CR^{2c}R^{2d}, CR^{2a}R^{2b} and CR^{2e}R^{2f} are independently selected from H, substituted or unsubstituted C₁-C₆alkyl, substituted or unsubstituted C₁-C₆heteroalkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), —C₁-C₆alkyl-(substituted or unsubstituted heteroaryl) and —C(═O)R^{B};
R^{B} of —C(═O)R^{B} is selected from substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), —C₁-C₆alkyl-(substituted or unsubstituted heteroaryl), or —NR^{D}R^{E};
R^{D} and R^{E} of NR^{D}R^{E} are independently selected from H, substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), or —C₁-C₆alkyl-(substituted or unsubstituted heteroaryl);
m of Formula (XXXIV) is selected from 0, 1 or 2;
—U— of Formula (XXXIV) is selected from —NHC(═O)—, —C(═O)NH—, —NHS(═O)₂—, —S(═O)₂NH—, —NHC(═O)NH—, —NH(C═O)O—, —O(C═O)NH—, or —NHS(═O)₂NH—;
R³ of Formula (XXXIV) is selected from C₁-C₃alkyl, or C₁-C₃fluoroalkyl;
R⁴ of Formula (XXXIV) is selected from —NHR⁵, —N(R⁵)₂, —N+(R⁵)₃ or —OR⁵;
each R⁵ of —NHR⁵, —N(R⁵)₂, —N+(R⁵)₃ and —OR⁵ is independently selected from H, C₁-C₆alkyl, C₁-C₃haloalkyl, C₁-C₃heteroalkyl and —C₁-C₃alkyl-(C₃-C₅cycloalkyl);
or:
R³ and R⁵ of Formula (XXXIV) together with the atoms to which they are attached form a substituted or unsubstituted 5-7 membered ring;
or:
R³ of Formula (XXIV) is bonded to a nitrogen atom of U to form a substituted or unsubstituted 5-7 membered ring;
R⁶ of Formula (XXIV) is selected from —NHC(═O)R⁷, —C(═O)NHR⁷, —NHS(═O)2R⁷, —S(═O)₂NHR⁷; —NHC(═O)NHR⁷, —NHS(═O)₂NHR⁷, —(C₁-C₃alkyl)-NHC(═O)R⁷, —(C₁-C₃alkyl)-C(═O)NHR⁷, —(C₁-C₃alkyl)-NHS(═O)₂R⁷, —(C₁-C₃alkyl)-S(═O)₂NHR⁷; —(C₁-C₃alkyl)-NHC(═O)NHR⁷, —(C₁-C₃alkyl)-NHS(═O)₂NHR⁷, substituted or unsubstituted C₂-C₁₀heterocycloalkyl, or substituted or unsubstituted heteroaryl;
each R⁷ of —NHC(═O)R⁷, —C(═O)NHR⁷, —NHS(═O)2R⁷, —S(═O)₂NHR⁷; —NHC(═O)NHR⁷, —NHS(═O)₂NHR⁷, —(C₁-C₃alkyl)-NHC(═O)R⁷, —(C₁-C₃alkyl)-C(═O)NHR⁷, —(C₁-C₃alkyl)-NHS(═O)2R⁷, —(C₁-C₃alkyl)-S(═O)2NHR⁷; —(C₁-C₃alkyl)-NHC(═O)NHR⁷, —(C₁-C₃alkyl)-NHS(═O)2NHR⁷ is independently selected from C₁-C₆alkyl, C₁-C₆haloalkyl, C₁-C₆heteroalkyl, a substituted or unsubstituted C₃-C₁₀cycloalkyl, a substituted or unsubstituted C₂-C₁₀heterocycloalkyl, a substituted or unsubstituted aryl, a substituted or unsubstituted heteroaryl, —C₁-C₆alkyl-(substituted or unsubstituted C₃-C₁₀cycloalkyl), —C₁-C₆alkyl-(substituted or unsubstituted C2-C10heterocycloalkyl, —C1-C6alkyl-(substituted or unsubstituted aryl), —C₁-C₆alkyl-(substituted or unsubstituted heteroaryl), —(CH2)p-CH(substituted or unsubstituted aryl)2, —(CH₂)ₚ—CH(substituted or unsubstituted heteroaryl)2, —(CH₂)_{P}—CH(substituted or unsubstituted aryl)(substituted or unsubstituted heteroaryl), -(substituted or unsubstituted aryl)-(substituted or unsubstituted aryl), -(substituted or unsubstituted aryl)-(substituted or unsubstituted heteroaryl), -(substituted or unsubstituted heteroaryl)-(substituted or unsubstituted aryl), or -(substituted or unsubstituted heteroaryl)-(substituted or unsubstituted heteroaryl);
p of R⁷ is selected from 0, 1 or 2;
R^{8a}, R^{8b}, R^{8c}, R^{8d}, R^{8e}, and R^{8f} of C(R^{8a})(R^{8b}), C(R^{8c})(R^{8d}) and C(R^{8e})(R^{8f}) are independently selected from H, C₁-C₆alkyl, C₁-C₆fluoroalkyl, C₁-C₆ alkoxy, C₁-C₆heteroalkyl, and substituted or unsubstituted aryl;
or:
R^{8a}, R^{8d}, R^{8e}, and R^{8f} of C(R^{8a})(R^{8b}), C(R^{8c})(R^{8d}) and C(R^{8e})(R^{8f}) are as defined above, and R^{8b} and R^{8c} together form a bond;
or:
R^{8a}, R^{8b}, R^{8d}, and R^{8f} of C(R^{8a})(R^{8b}), C(R^{8c})(R^{8d}) and C(R^{8e})(R^{8f}) are as defined above, and R^{8c} and R^{8e} together form a bond;
or:
R^{8a}, R^{8d}, R^{8e}, and R^{8f} of C(R^{8a})(R^{8b}), C(R^{8c})(R^{8d}) and C(R^{8e})(R^{8f}) are as defined above, and R^{8b} and R^{8c} together with the atoms to which they are attached form a substituted or unsubstituted fused 5-7 membered saturated, or partially saturated carbocyclic ring or heterocyclic ring comprising 1-3 heteroatoms selected from S, O and N, a substituted or unsubstituted fused 5-10 membered aryl ring, or a substituted or unsubstituted fused 5-10 membered heteroaryl ring comprising 1-3 heteroatoms selected from S, O and N;
or:
R^{8a}, R^{8b}, R^{8e}, and R^{8f} of C(R^{8a})(R^{8b}), C(R^{8c})(R^{8d}) and C(R^{8e})(R^{8f}) are as defined above, and R^{8c} and R^{8e} together with the atoms to which they are attached form a substituted or unsubstituted fused 5-7 membered saturated, or partially saturated carbocyclic ring or heterocyclic ring comprising 1-3 heteroatoms selected from S, O and N, a substituted or unsubstituted fused 5-10 membered aryl ring, or a substituted or unsubstituted fused 5-10 membered heteroaryl ring comprising 1-3 heteroatoms selected from S, O and N;
or:
R^{8c}, R^{8d}, R^{8e}, and R^{8f} of C(R^{8c})(R^{8d}) and C(R^{8e})(R^{8f}) are as defined above, and R^{8a} and R^{8b} together with the atoms to which they are attached form a substituted or unsubstituted saturated, or partially saturated 3-7 membered spirocycle or heterospirocycle comprising 1-3 heteroatoms selected from S, O and N;
or:
R^{8a}, R^{8b}, R^{8e}, and R^{8f} of C(R^{8a})(R^{8b}) and C(R^{8e})(R^{8f}) are as defined above, and R^{8c} and R^{8d} together with the atoms to which they are attached form a substituted or unsubstituted saturated, or partially saturated 3-7 membered spirocycle or heterospirocycle comprising 1-3 heteroatoms selected from S, O and N;
or:
R^{8a}, R^{8b}, R^{8c}, and R^{8d} of C(R^{8a})(R^{8b}) and C(R^{8c})(R^{8d}) are as defined above, and R^{8e} and R^{8f} together with the atoms to which they are attached form a substituted or unsubstituted saturated, or partially saturated 3-7 membered spirocycle or heterospirocycle comprising 1-3 heteroatoms selected from S, O and N;
or:
where each substituted alkyl, heteroalkyl, fused ring, spirocycle, heterospirocycle, cycloalkyl, heterocycloalkyl, aryl or heteroaryl is substituted with 1-3 R⁹; and
each R⁹ of R^{8a}, R^{8b}, R^{8c}, R^{8d}, R^{8e}, and R^{8f} is independently selected from halogen, —OH, —SH, (C═O), CN, C₁-C₄alkyl, C₁-C₄fluoroalkyl, C₁-C₄ alkoxy, C₁-C₄ fluoroalkoxy, —NH₂, —NH(C₁-C₄alkyl), —NH(C₁-C₄alkyl)₂, —C(═O)OH, —C(═O)NH₂, —C(═O)C₁-C₃alkyl, —S(═O)₂CH₃, —NH(C₁-C₄alkyl)-OH, —NH(C₁-C₄alkyl)-O—(C—C₄alkyl), —O(C₁-C₄alkyl)-NH2; —O(C₁-C₄alkyl)-NH—(C₁-C₄alkyl), and —O(C₁-C₄alkyl)-N—(C₁-C₄alkyl)₂, or two R⁹ together with the atoms to which they are attached form a methylene dioxy or ethylene dioxy ring substituted or unsubstituted with halogen, —OH, or C₁-C₃alkyl.

In any of the compounds described herein, the ILM can have the structure of Formula (XXXV), (XXXVI) or (XXXVII), which is derived from the IAP ligands described in Vamos, M., et al., Expedient synthesis of highly potent antagonists of inhibitor of apoptosis proteins (IAPs) with unique selectivity for ML-IAP, ACS Chem. Biol., 8(4), 725-32 (2013), or an unnatural mimetic thereof:

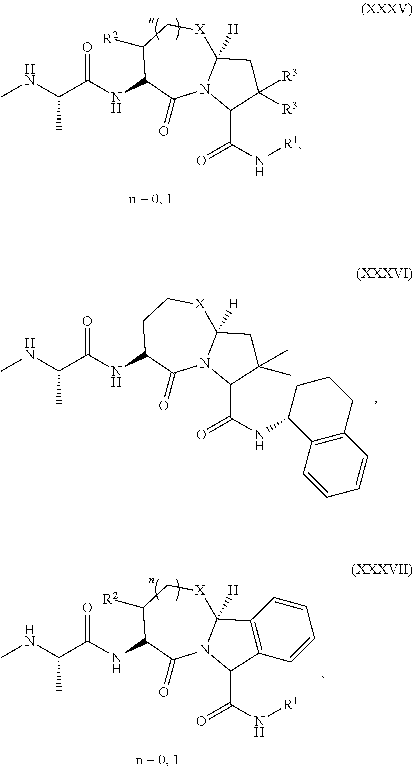

wherein:

R² of Formulas (XXXV) and (XXXVII) are independently selected from H or ME;

R³ and R⁴ of Formula (XXXV) are independently selected from H or ME;

X of Formulas (XXXV) and (XXXVII) is independently selected from O or S; and

R¹ of Formulas (XXXV) and (XXXVII) is selected from:

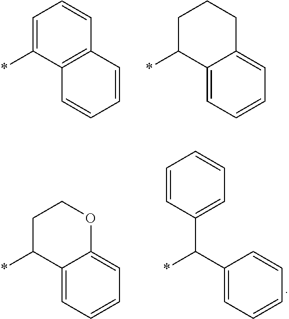

In a particular embodiment, the ILM has a structure according to Formula (XXXVIII):

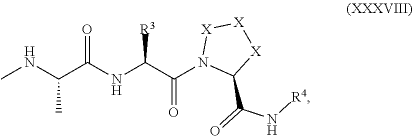

wherein R³ and R⁴ of Formula (XXXVIII) are independently selected from H or ME;

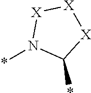

is a 5-member heteocycle selected from:

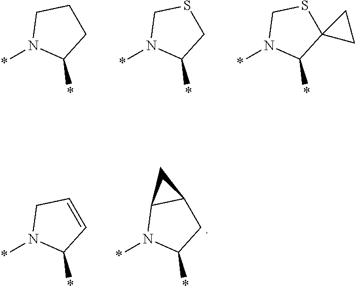

In a particular embodiment, the

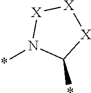

### of Formula (XXXVIII) is

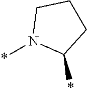 [SMILES: *[C@@H]1CCCN1*]

In a particular embodiment, the ILM has a structure and attached to a linker group L as shown below:

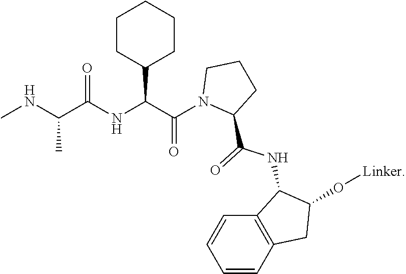

In any of the compounds described herein, the ILM can have the structure of Formula (XXXIX) or (XL), which is based on the IAP ligands described in Hennessy, E J, et al., Discovery of aminopiperidine-based Smac mimetics as IAP antagonists, Bioorg. Med. Chem. Lett., 22(4), 1960-4 (2012), or an unnatural mimetic thereof:

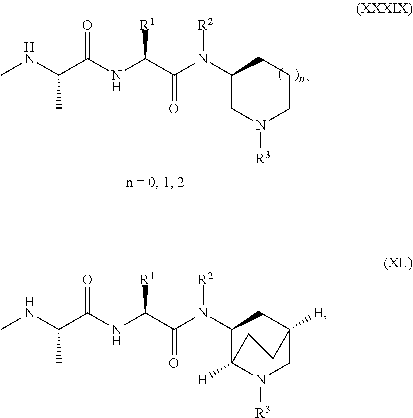

wherein:

R¹ of Formulas (XXXIX) and (XL) is selected from:

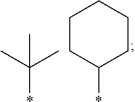

R² of Formulas (XXXIX) and (XL) is selected from H or Me;

R³ of Formulas (XXXIX) and (XL) is selected from:

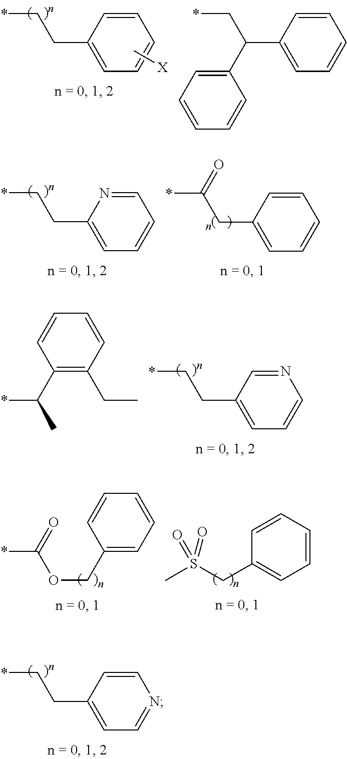

X of is selected from H, halogen, methyl, methoxy, hydroxy, nitro or trifluoromethyl.

In any of the compounds described herein, the ILM can have the structure of and be chemically linked to the linker as shown in Formula (XLI) or (XLII), or an unnatural mimetic thereof:

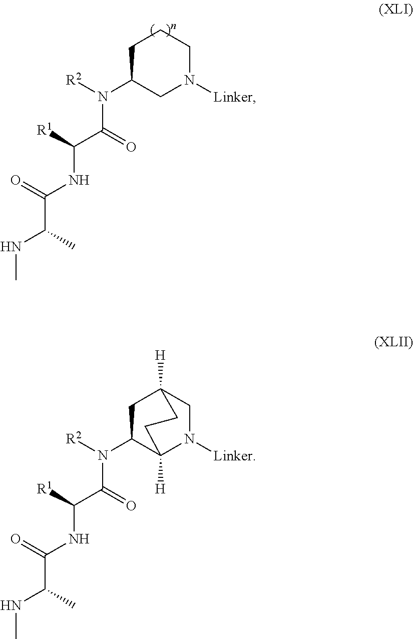

In any of the compounds described herein, the ILM can have the structure of Formula (XLIII), which is based on the IAP ligands described in Cohen, F, et al., Orally bioavailable antagonists of inhibitor of apoptosis proteins based on an azabicyclooctane scaffold, J. Med. Chem., 52(6), 1723-30 (2009), or an unnatural mimetic thereof:

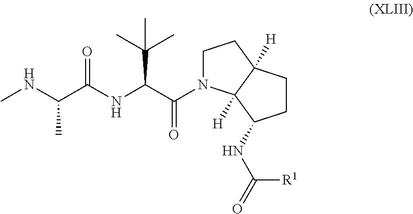

wherein:

R₁ of Formulas (XLIII) is selected from:

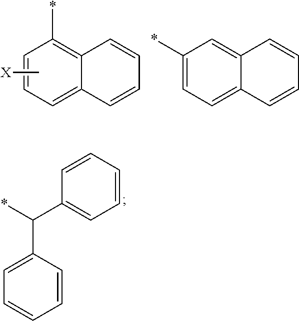

X of

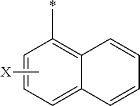

is selected from H, fluoro, methyl or methoxy.

In a particular embodiment, the ILM is represented by the following structure:

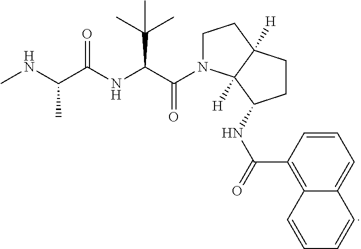 [SMILES: CN[C@@H](C)C(=O)N[C@H](C(=O)N1CC[C@H]2CC[C@H](NC(=O)c3cccc4ccccc34)[C@H]21)C(C)(C)C]

In a particular embodiment, the ILM is selected from the group consisting of, and which the chemical link between the ILM and linker group L is shown:

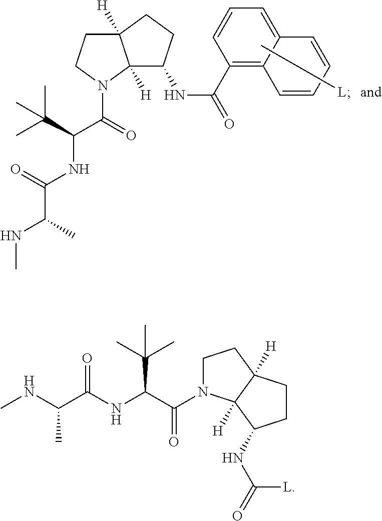

In any of the compounds described herein, the ILM is selected from the group consisting of the structures below, which are based on the IAP ligands described in Asano, M, et al., Design, sterioselective synthesis, and biological evaluation of novel tri-cyclic compounds as inhibitor of apoptosis proteins (IAP) antagonists, Bioorg. Med. Chem., 21(18): 5725-37 (2013), or an unnatural mimetic thereof:

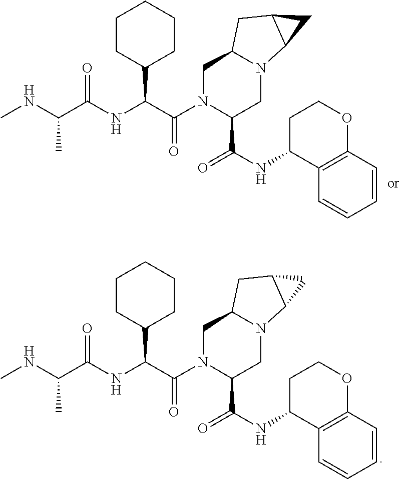 [SMILES: CN[C@@H](C)C(=O)N[C@H](C(=O)N1C[C@H]2C[C@H]3C[C@H]3N2C[C@H]1C(=O)N[C@@H]1CCOc2ccccc21)C1CCCCC1 ; CN[C@@H](C)C(=O)N[C@H](C(=O)N1C[C@H]2C[C@@H]3C[C@@H]3N2C[C@H]1C(=O)N[C@@H]1CCOc2ccccc21)C1CCCCC1]

In a particular embodiment, the ILM is selected from the group consisting of, and which the chemical link between the ILM and linker group L is shown:

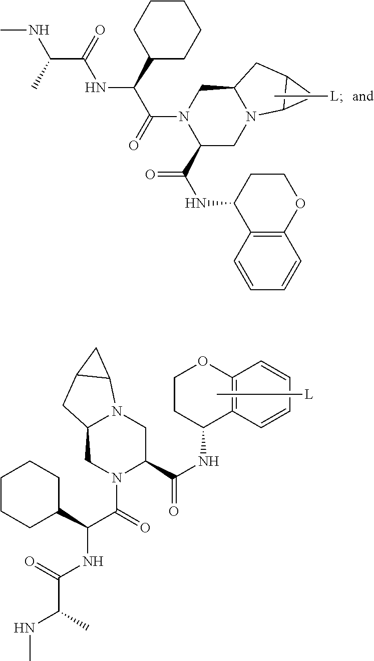

In any of the compounds described herein, the ILM can have the structure of Formula (LVIII), which is based on the IAP ligands described in Asano, M, et al., Design, sterioselective synthesis, and biological evaluation of novel tri-cyclic compounds as inhibitor of apoptosis proteins (IAP) antagonists, Bioorg. Med. Chem., 21(18): 5725-37 (2013), or an unnatural mimetic thereof:

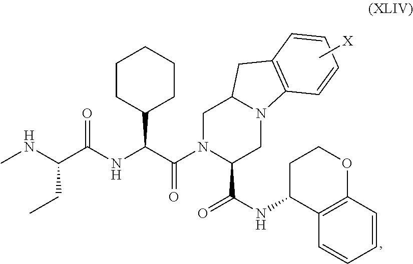

wherein X of Formula (XLIV) is one or two substituents independently selected from H, halogen or cyano.

In any of the compounds described herein, the ILM can have the structure of and be chemically linked to the linker group L as shown in Formula (XLV) or (XLVI), or an unnatural mimetic thereof:

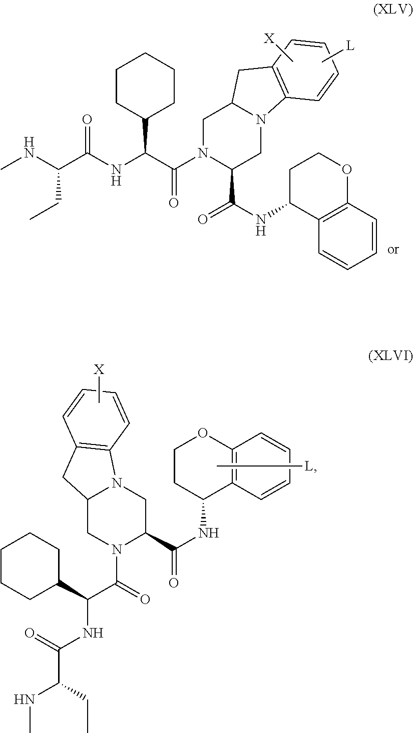

wherein X of Formula (XLV) and (XLVI) is one or two substituents independently selected from H, halogen or cyano, and; and L of Formulas (XLV) and (XLVI) is a linker group as described herein.

In any of the compounds described herein, the ILM can have the structure of Formula (XLVII), which is based on the IAP ligands described in Ardecky, R J, et al., Design, synthesis and evaluation of inhibitor of apoptosis (IAP) antagonists that are highly selective for the BIR2 domain of XIAP, Bioorg. Med. Chem., 23(14): 4253-7 (2013), or an unnatural mimetic thereof:

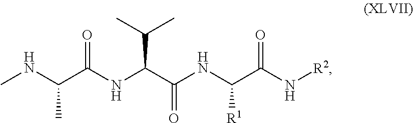

wherein:

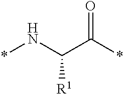

of Formula (XLVII) is a natural or unnatural amino acid; and R² of Formula (XLVII) is selected from:

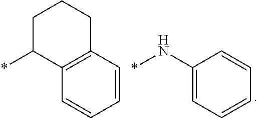

In any of the compounds described herein, the ILM can have the structure of and be chemically linked to the linker group L as shown in Formula (XLVIII) or (XLIX), or an unnatural mimetic thereof:

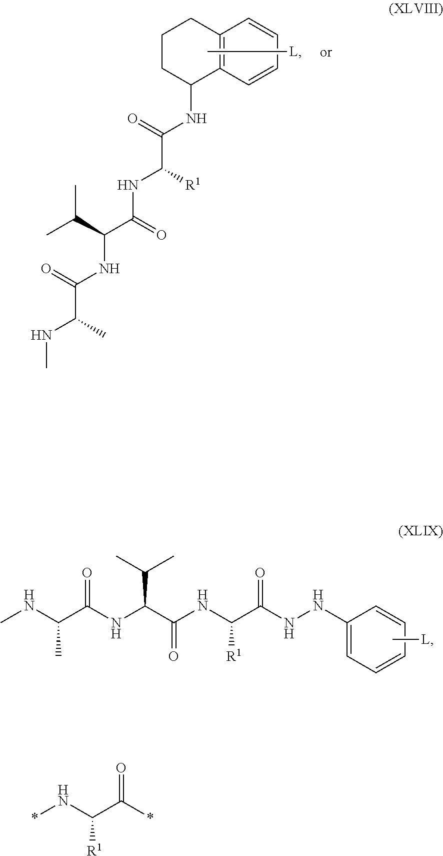

of Formula s (XLVIII) and (XLIX) is a natural or unnatural amino acid; and L of Formula s (XLVIII) and (XLIX) is a linker group as described herein.

In any of the compounds described herein, the ILM can have the structure selected from the group consisting of, which is based on the IAP ligands described in Wang, J, et al., Discovery of novel second mitochondrial-derived activator of caspase mimetics as selective inhibitor or apoptosis protein inhibitors, J. Pharmacol. Exp. Ther., 349(2): 319-29 (2014), or an unnatural mimetic thereof:

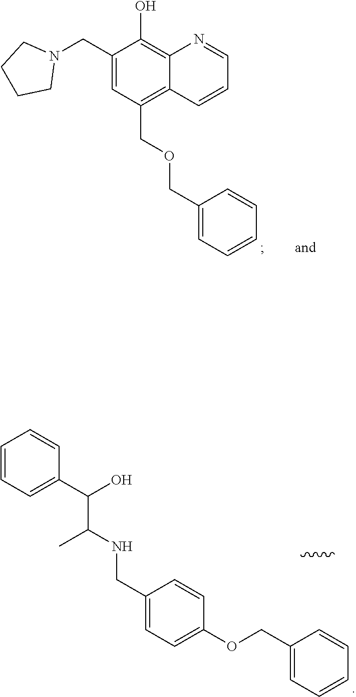

In any of the compounds described herein, the ILM has a structure according to Formula (LXIX), which is based on the IAP ligands described in Hird, A W, et al., Structure-based design and synthesis of tricyclic IAP (Inhibitors of Apoptosis Proteins) inhibitors, Bioorg. Med. Chem. Lett., 24(7): 1820-4 (2014), or an unnatural mimetic thereof:

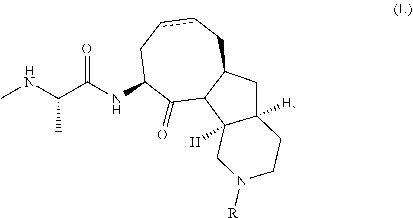

wherein R of Formula (L) is selected from the group consisting of:

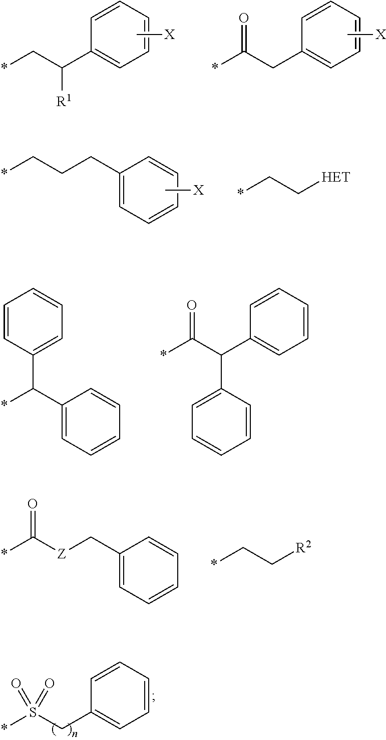

### R1 of

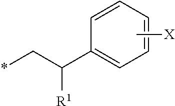

is selected from H or Me;

### R2 of

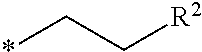

is selected from alkyl or cycloalkyl;

### X of

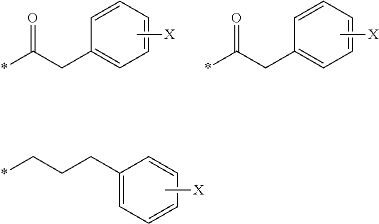

is 1-2 substitutents independently selected from halogen, hydroxy, methoxy, nitro and trifluoromethyl

### Z of

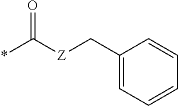

### is O or NH;

### HET of

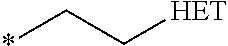

is mono- or fused bicyclic heteroaryl; and [symbol]
of Formula (L) is an optional double bond.
In a particular embodiment, the ILM of the compound has a chemical structure selected from the group consisting of:

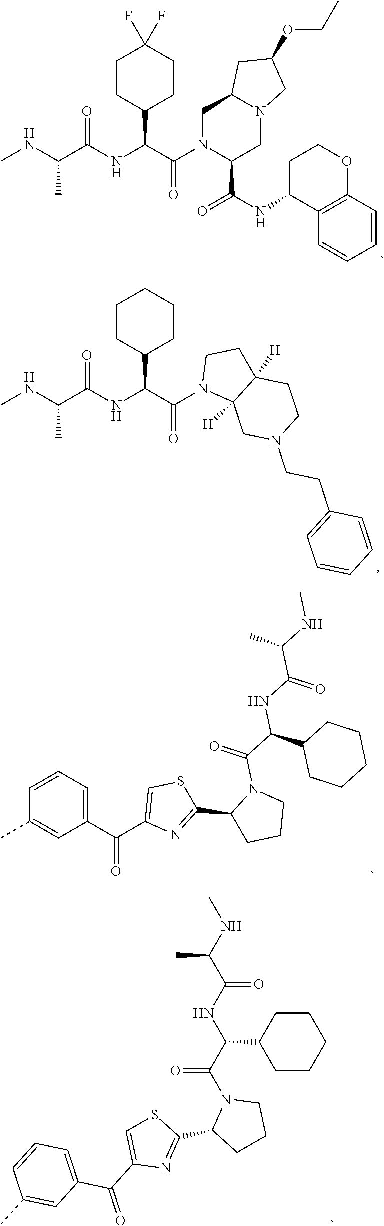

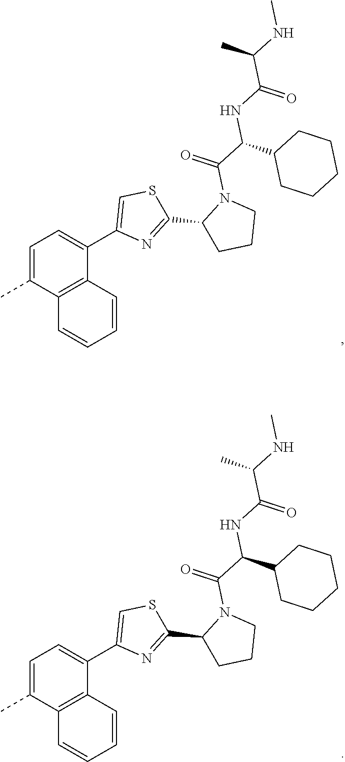

The term “independently” is used herein to indicate that the variable, which is independently applied, varies independently from application to application.

The term “alkyl” shall mean within its context a linear, branch-chained or cyclic fully saturated hydrocarbon radical or alkyl group, preferably a C₁-C₁₀, more preferably a C₁-C₆, alternatively a C₁-C₃ alkyl group, which may be optionally substituted. Examples of alkyl groups are methyl, ethyl, n-butyl, sec-butyl, n-hexyl, n-heptyl, n-octyl, n-nonyl, n-decyl, isopropyl, 2-methylpropyl, cyclopropyl, cyclopropylmethyl, cyclobutyl, cyclopentyl, cyclopentylethyl, cyclohexylethyl and cyclohexyl, among others. In certain embodiments, the alkyl group is end-capped with a halogen group (At, Br, Cl, F, or I). In certain preferred embodiments, compounds according to the present disclosure which may be used to covalently bind to dehalogenase enzymes. These compounds generally contain a side chain (often linked through a polyethylene glycol group) which terminates in an alkyl group which has a halogen substituent (often chlorine or bromine) on its distal end which results in covalent binding of the compound containing such a moiety to the protein.

The term “Alkenyl” refers to linear, branch-chained or cyclic C₂-C₁₀ (preferably C₂-C₆) hydrocarbon radicals containing at least one C═C bond.

The term “Alkynyl” refers to linear, branch-chained or cyclic C₂-C₁₀ (preferably C₂-C₆) hydrocarbon radicals containing at least one C═C bond.

The term “alkylene” when used, refers to a —(CH₂)ₙ— group (n is an integer generally from 0-6), which may be optionally substituted. When substituted, the alkylene group preferably is substituted on one or more of the methylene groups with a C₁-C₆ alkyl group (including a cyclopropyl group or a t-butyl group), but may also be substituted with one or more halo groups, preferably from 1 to 3 halo groups or one or two hydroxyl groups, O—(C₁-C₆ alkyl) groups or amino acid sidechains as otherwise disclosed herein. In certain embodiments, an alkylene group may be substituted with a urethane or alkoxy group (or other group) which is further substituted with a polyethylene glycol chain (of from 1 to 10, preferably 1 to 6, often 1 to 4 ethylene glycol units) to which is substituted (preferably, but not exclusively on the distal end of the polyethylene glycol chain) an alkyl chain substituted with a single halogen group, preferably a chlorine group. In still other embodiments, the alkylene (often, a methylene) group, may be substituted with an amino acid sidechain group such as a sidechain group of a natural or unnatural amino acid, for example, alanine, P-alanine, arginine, asparagine, aspartic acid, cysteine, cystine, glutamic acid, glutamine, glycine, phenylalanine, histidine, isoleucine, lysine, leucine, methionine, proline, serine, threonine, valine, tryptophan or tyrosine.

The term “unsubstituted” shall mean substituted only with hydrogen atoms. A range of carbon atoms which includes C₀ means that carbon is absent and is replaced with H. Thus, a range of carbon atoms which is C₀-C₆ includes carbons atoms of 1, 2, 3, 4, 5 and 6 and for C₀, H stands in place of carbon.

The term “substituted” or “optionally substituted” shall mean independently (i.e., where more than substituent occurs, each substituent is independent of another substituent) one or more substituents (independently up to five substitutents, preferably up to three substituents, often 1 or 2 substituents on a moiety in a compound according to the present disclosure and may include substituents which themselves may be further substituted) at a carbon (or nitrogen) position anywhere on a molecule within context, and includes as substituents hydroxyl, thiol, carboxyl, cyano (C≡N), nitro (NO₂), halogen (preferably, 1, 2 or 3 halogens, especially on an alkyl, especially a methyl group such as a trifluoromethyl), an alkyl group (preferably, C₁-C₁₀, more preferably, C₁-C₆), aryl (especially phenyl and substituted phenyl for example benzyl or benzoyl), alkoxy group (preferably, C₁-C₆ alkyl or aryl, including phenyl and substituted phenyl), thioether (C₁-C₆ alkyl or aryl), acyl (preferably, C₁-C₆ acyl), ester or thioester (preferably, C₁-C₆ alkyl or aryl) including alkylene ester (such that attachment is on the alkylene group, rather than at the ester function which is preferably substituted with a C₁-C₆ alkyl or aryl group), preferably, C₁-C₆ alkyl or aryl, halogen (preferably, F or Cl), amine (including a five- or six-membered cyclic alkylene amine, further including a C₁-C₆ alkyl amine or a C₁-C₆ dialkyl amine which alkyl groups may be substituted with one or two hydroxyl groups) or an optionally substituted —N(C₀-C₆ alkyl)C(O)(O—C₁-C₆ alkyl) group (which may be optionally substituted with a polyethylene glycol chain to which is further bound an alkyl group containing a single halogen, preferably chlorine substituent), hydrazine, amido, which is preferably substituted with one or two C₁-C₆ alkyl groups (including a carboxamide which is optionally substituted with one or two C₁-C₆ alkyl groups), alkanol (preferably, C₁-C₆ alkyl or aryl), or alkanoic acid (preferably, C₁-C₆ alkyl or aryl). Substituents according to the present disclosure may include, for example —SiR₁R₂R₃ groups where each of R₁ and R₂ is as otherwise described herein and R₃ is H or a C₁-C₆ alkyl group, preferably R₁, R₂, R₃ in this context is a C₁-C₃ alkyl group (including an isopropyl or t-butyl group). Each of the above-described groups may be linked directly to the substituted moiety or alternatively, the substituent may be linked to the substituted moiety (preferably in the case of an aryl or heteraryl moiety) through an optionally substituted —(CH₂)ₘ— or alternatively an optionally substituted —(OCH₂)ₘ—, —(OCH₂CH₂)ₘ— or —(CH₂CH₂O)ₘ— group, which may be substituted with any one or more of the above-described substituents. Alkylene groups —(CH₂)ₘ— or —(CH₂)ₙ— groups or other chains such as ethylene glycol chains, as identified above, may be substituted anywhere on the chain. Preferred substitutents on alkylene groups include halogen or C₁-C₆ (preferably C₁-C₃) alkyl groups, which may be optionally substituted with one or two hydroxyl groups, one or two ether groups (O—C₁-C₆ groups), up to three halo groups (preferably F), or a sideshain of an amino acid as otherwise described herein and optionally substituted amide (preferably carboxamide substituted as described above) or urethane groups (often with one or two C₀-C₆ alkyl substitutents, which group(s) may be further substituted). In certain embodiments, the alkylene group (often a single methylene group) is substituted with one or two optionally substituted C₁-C₆ alkyl groups, preferably C₁-C₄ alkyl group, most often methyl or O-methyl groups or a sidechain of an amino acid as otherwise described herein. In the present disclosure, a moiety in a molecule may be optionally substituted with up to five substituents, preferably up to three substituents. Most often, in the present disclosure moieties which are substituted are substituted with one or two substituents.

The term “substituted” (each substituent being independent of any other substituent) shall also mean within its context of use C₁-C₆ alkyl, C₁-C₆ alkoxy, halogen, amido, carboxamido, sulfone, including sulfonamide, keto, carboxy, C₁-C₆ ester (oxyester or carbonylester), C₁-C₆ keto, urethane —O—C(O)—NR₁R₂ or —N(R₁)—C(O)—O—R₁, nitro, cyano and amine (especially including a C₁-C₆ alkylene-NR₁R₂, a mono- or di-C₁-C₆ alkyl substituted amines which may be optionally substituted with one or two hydroxyl groups). Each of these groups contain unless otherwise indicated, within context, between 1 and 6 carbon atoms. In certain embodiments, preferred substituents will include for example, —NH—, —NHC(O)—, —O—, ═O, —(CH₂)ₘ— (here, m and n are in context, 1, 2, 3, 4, 5 or 6), —S—, —S(O)—, SO₂— or —NH—C(O)—NH—, —(CH₂)ₙOH, —(CH₂)ₙSH, —(CH₂)ₙCOOH, C₁-C₆ alkyl, —(CH₂)ₙO—(C₁-C₆ alkyl), —(CH₂)ₙC(O)—(C₁-C₆ alkyl), —(CH₂)ₙOC(O)—(C₁-C₆ alkyl), —(CH₂)ₙC(O)O—(C₁-C₆ alkyl), —(CH₂)ₙNHC(O)—R₁, —(CH₂)ₙC(O)—NR₁R₂, —(OCH₂)ₙOH, —(CH₂O)ₙCOOH, C₁-C₆ alkyl, —(OCH₂)ₙO—(C₁-C₆ alkyl), —(CH₂O)ₙC(O)—(C₁-C₆ alkyl), —(OCH₂)ₙNHC(O)—R₁, —(CH₂O)ₙC(O)—NR₁R₂, —S(O)₂—R_{S}, —S(O)—R_{S} (R_{S} is C₁-C₆ alkyl or a —(CH₂)ₘ—NR₁R₂ group), NO₂, CN or halogen (F, Cl, Br, I, preferably F or Cl), depending on the context of the use of the substituent. R₁ and R₂ are each, within context, H or a C₁-C₆ alkyl group (which may be optionally substituted with one or two hydroxyl groups or up to three halogen groups, preferably fluorine). The term “substituted” shall also mean, within the chemical context of the compound defined and substituent used, an optionally substituted aryl or heteroaryl group or an optionally substituted heterocyclic group as otherwise described herein. Alkylene groups may also be substituted as otherwise disclosed herein, preferably with optionally substituted C₁-C₆ alkyl groups (methyl, ethyl or hydroxymethyl or hydroxyethyl is preferred, thus providing a chiral center), a sidechain of an amino acid group as otherwise described herein, an amido group as described hereinabove, or a urethane group O—C(O)—NR₁R₂ group where R₁ and R₂ are as otherwise described herein, although numerous other groups may also be used as substituents. Various optionally substituted moieties may be substituted with 3 or more substituents, preferably no more than 3 substituents and preferably with 1 or 2 substituents. It is noted that in instances where, in a compound at a particular position of the molecule substitution is required (principally, because of valency), but no substitution is indicated, then that substituent is construed or understood to be H, unless the context of the substitution suggests otherwise.

The term “aryl” or “aromatic”, in context, refers to a substituted (as otherwise described herein) or unsubstituted monovalent aromatic radical having a single ring (e.g., benzene, phenyl, benzyl) or condensed rings (e.g., naphthyl, anthracenyl, phenanthrenyl, etc.) and can be bound to the compound according to the present disclosure at any available stable position on the ring(s) or as otherwise indicated in the chemical structure presented. Other examples of aryl groups, in context, may include heterocyclic aromatic ring systems, “heteroaryl” groups having one or more nitrogen, oxygen, or sulfur atoms in the ring (moncyclic) such as imidazole, furyl, pyrrole, furanyl, thiene, thiazole, pyridine, pyrimidine, pyrazine, triazole, oxazole or fused ring systems such as indole, quinoline, indolizine, azaindolizine, benzofurazan, etc., among others, which may be optionally substituted as described above. Among the heteroaryl groups which may be mentioned include nitrogen-containing heteroaryl groups such as pyrrole, pyridine, pyridone, pyridazine, pyrimidine, pyrazine, pyrazole, imidazole, triazole, triazine, tetrazole, indole, isoindole, indolizine, azaindolizine, purine, indazole, quinoline, dihydroquinoline, tetrahydroquinoline, isoquinoline, dihydroisoquinoline, tetrahydroisoquinoline, quinolizine, phthalazine, naphthyridine, quinoxaline, quinazoline, cinnoline, pteridine, imidazopyridine, imidazotriazine, pyrazinopyridazine, acridine, phenanthridine, carbazole, caibazoline, pyrimidine, phenanthroline, phenacene, oxadiazole, benzimidazole, pyrrolopyridine, pyrrolopyrimidine and pyridopyrimidine; sulfur-containing aromatic heterocycles such as thiophene and benzothiophene; oxygen-containing aromatic heterocycles such as furan, pyran, cyclopentapyran, benzofuran and isobenzofuran; and aromatic heterocycles comprising 2 or more hetero atoms selected from among nitrogen, sulfur and oxygen, such as thiazole, thiadizole, isothiazole, benzoxazole, benzothiazole, benzothiadiazole, phenothiazine, isoxazole, furazan, phenoxazine, pyrazoloxazole, imidazothiazole, thienofuran, furopyrrole, pyridoxazine, furopyridine, furopyrimidine, thienopyrimidine and oxazole, among others, all of which may be optionally substituted.

The term “substituted aryl” refers to an aromatic carbocyclic group comprised of at least one aromatic ring or of multiple condensed rings at least one of which being aromatic, wherein the ring(s) are substituted with one or more substituents. For example, an aryl group can comprise a substituent(s) selected from: —(CH₂)ₙOH, —(CH₂)ₙ—O—(C₁-C₆)alkyl, —(CH₂)ₙ—O—(CH₂)ₙ—(C₁-C₆)alkyl, —(CH₂)ₙ—C(O)(C₀-C₆) alkyl, —(CH₂)ₙ—C(O)O(C₀-C₆)alkyl, —(CH₂)ₙ—OC(O)(C₀-C₆)alkyl, amine, mono- or di-(C₁-C₆ alkyl) amine wherein the alkyl group on the amine is optionally substituted with 1 or 2 hydroxyl groups or up to three halo (preferably F, Cl) groups, OH, COOH, C₁-C₆ alkyl, preferably CH₃, CF₃, OMe, OCF₃, NO₂, or CN group (each of which may be substituted in ortho-, meta- and/or para-positions of the phenyl ring, preferably para-), an optionally substituted phenyl group (the phenyl group itself is preferably connected to a PTM group, including a ULM group, via a linker group), and/or at least one of F, Cl, OH, COOH, CH₃, CF₃, OMe, OCF₃, NO₂, or CN group (in ortho-, meta- and/or para-positions of the phenyl ring, preferably para-), a naphthyl group, which may be optionally substituted, an optionally substituted heteroaryl, preferably an optionally substituted isoxazole including a methylsubstituted isoxazole, an optionally substituted oxazole including a methylsubstituted oxazole, an optionally substituted thiazole including a methyl substituted thiazole, an optionally substituted isothiazole including a methyl substituted isothiazole, an optionally substituted pyrrole including a methylsubstituted pyrrole, an optionally substituted imidazole including a methylimidazole, an optionally substituted benzimidazole or methoxybenzylimidazole, an optionally substituted oximidazole or methyloximidazole, an optionally substituted diazole group, including a methyldiazole group, an optionally substituted triazole group, including a methylsubstituted triazole group, an optionally substituted pyridine group, including a halo-(preferably, F) or methylsubstitutedpyridine group or an oxapyridine group (where the pyridine group is linked to the phenyl group by an oxygen), an optionally substituted furan, an optionally substituted benzofuran, an optionally substituted dihydrobenzofuran, an optionally substituted indole, indolizine or azaindolizine (2, 3, or 4-azaindolizine), an optionally substituted quinoline, and combinations thereof.

“Carboxyl” denotes the group —C(O)OR, where R is hydrogen, alkyl, substituted alkyl, aryl, substituted aryl, heteroaryl or substituted heteroaryl, whereas these generic substituents have meanings which are identical with definitions of the corresponding groups defined herein.

The term “heteroaryl” or “hetaryl” can mean but is in no way limited to an optionally substituted quinoline (which may be attached to the pharmacophore or substituted on any carbon atom within the quinoline ring), an optionally substituted indole (including dihydroindole), an optionally substituted indolizine, an optionally substituted azaindolizine (2, 3 or 4-azaindolizine) an optionally substituted benzimidazole, benzodiazole, benzoxofuran, an optionally substituted imidazole, an optionally substituted isoxazole, an optionally substituted oxazole (preferably methyl substituted), an optionally substituted diazole, an optionally substituted triazole, a tetrazole, an optionally substituted benzofuran, an optionally substituted thiophene, an optionally substituted thiazole (preferably methyl and/or thiol substituted), an optionally substituted isothiazole, an optionally substituted triazole (preferably a 1,2,3-triazole substituted with a methyl group, a triisopropylsilyl group, an optionally substituted —(CH₂)ₘ—O—C₁-C₆ alkyl group or an optionally substituted —(CH₂)ₘ—C(O)—O—C₁-C₆ alkyl group), an optionally substituted pyridine (2-, 3, or 4-pyridine) or a group according to the chemical structure:

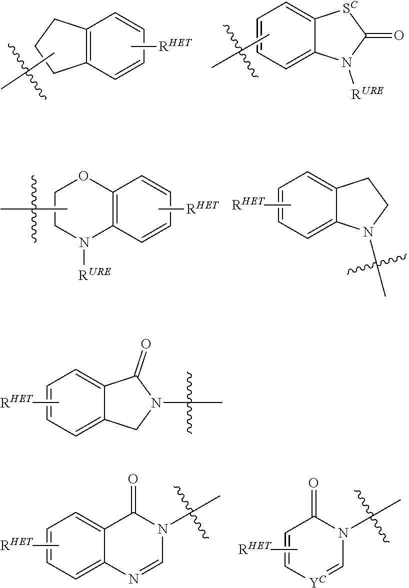

wherein:
S^{c} is CHR^{SS}, NR^{URE}, or O;
R^{HET} is H, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl);
R^{SS} is H, CN, NO₂, halo (preferably F or Cl), optionally substituted C₁-C₆ alkyl (preferably substituted with one or two hydroxyl groups or up to three halo groups), optionally substituted O—(C₁-C₆ alkyl) (preferably substituted with one or two hydroxyl groups or up to three halo groups) or an optionally substituted —C(O)(C₁-C₆ alkyl) (preferably substituted with one or two hydroxyl groups or up to three halo groups);
R^{URE} is H, a C₁-C₆ (preferably H or C₁-C₃ alkyl) or a —C(O)(C₁-C₆ alkyl), each of which groups is optionally substituted with one or two hydroxyl groups or up to three halogen, preferably fluorine groups, or an optionally substituted heterocycle, for example piperidine, morpholine, pyrrolidine, tetrahydrofuran, tetrahydrothiophene, piperidine, piperazine, each of which is optionally substituted, and
Y^{C} is N or C—R^{YC}, where R^{YC} is H, OH, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl).

The terms “aralkyl” and “heteroarylalkyl” refer to groups that comprise both aryl or, respectively, heteroaryl as well as alkyl and/or heteroalkyl and/or carbocyclic and/or heterocycloalkyl ring systems according to the above definitions.

The term “arylalkyl” as used herein refers to an aryl group as defined above appended to an alkyl group defined above. The arylalkyl group is attached to the parent moiety through an alkyl group wherein the alkyl group is one to six carbon atoms. The aryl group in the arylalkyl group may be substituted as defined above.

The term “Heterocycle” refers to a cyclic group which contains at least one heteroatom, e.g., N, O or S, and may be aromatic (heteroaryl) or non-aromatic. Thus, the heteroaryl moieties are subsumed under the definition of heterocycle, depending on the context of its use. Exemplary heteroaryl groups are described hereinabove.

Exemplary heterocyclics include: azetidinyl, benzimidazolyl, 1,4-benzodioxanyl, 1,3-benzodioxolyl, benzoxazolyl, benzothiazolyl, benzothienyl, dihydroimidazolyl, dihydropyranyl, dihydrofuranyl, dioxanyl, dioxolanyl, ethyleneurea, 1,3-dioxolane, 1,3-dioxane, 1,4-dioxane, furyl, homopiperidinyl, imidazolyl, imidazolinyl, imidazolidinyl, indolinyl, indolyl, isoquinolinyl, isothiazolidinyl, isothiazolyl, isoxazolidinyl, isoxazolyl, morpholinyl, naphthyridinyl, oxazolidinyl, oxazolyl, pyridone, 2-pyrrolidone, pyridine, piperazinyl, N-methylpiperazinyl, piperidinyl, phthalimide, succinimide, pyrazinyl, pyrazolinyl, pyridyl, pyrimidinyl, pyrrolidinyl, pyrrolinyl, pyrrolyl, quinolinyl, tetrahydrofuranyl, tetrahydropyranyl, tetrahydroquinoline, thiazolidinyl, thiazolyl, thienyl, tetrahydrothiophene, oxane, oxetanyl, oxathiolanyl, thiane among others.

Heterocyclic groups can be optionally substituted with a member selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxy, carboxyalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SOaryl, —SO— heteroaryl, —SO2-alkyl, —SO2-substituted alkyl, —SO2-aryl, oxo (═O), and —SO2-heteroaryl. Such heterocyclic groups can have a single ring or multiple condensed rings. Examples of nitrogen heterocycles and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, morpholino, piperidinyl, tetrahydrofuranyl, and the like as well as N-alkoxy-nitrogen containing heterocycles. The term “heterocyclic” also includes bicyclic groups in which any of the heterocyclic rings is fused to a benzene ring or a cyclohexane ring or another heterocyclic ring (for example, indolyl, quinolyl, isoquinolyl, tetrahydroquinolyl, and the like).

The term “cycloalkyl” can mean but is in no way limited to univalent groups derived from monocyclic or polycyclic alkyl groups or cycloalkanes, as defined herein, e.g., saturated monocyclic hydrocarbon groups having from three to twenty carbon atoms in the ring, including, but not limited to, cyclopropyl, cyclobutyl, cyclopentyl, cyclohexyl, cycloheptyl and the like. The term “substituted cycloalkyl” can mean but is in no way limited to a monocyclic or polycyclic alkyl group and being substituted by one or more substituents, for example, amino, halogen, alkyl, substituted alkyl, carbyloxy, carbylmercapto, aryl, nitro, mercapto or sulfo, whereas these generic substituent groups have meanings which are identical with definitions of the corresponding groups as defined in this legend.

“Heterocycloalkyl” refers to a monocyclic or polycyclic alkyl group in which at least one ring carbon atom of its cyclic structure being replaced with a heteroatom selected from the group consisting of N, O, S or P. “Substituted heterocycloalkyl” refers to a monocyclic or polycyclic alkyl group in which at least one ring carbon atom of its cyclic structure being replaced with a heteroatom selected from the group consisting of N, O, S or P and the group is containing one or more substituents selected from the group consisting of halogen, alkyl, substituted alkyl, carbyloxy, carbylmercapto, aryl, nitro, mercapto or sulfo, whereas these generic substituent group have meanings which are identical with definitions of the corresponding groups as defined in this legend.

The term “hydrocarbyl” shall mean a compound which contains carbon and hydrogen and which may be fully saturated, partially unsaturated or aromatic and includes aryl groups, alkyl groups, alkenyl groups and alkynyl groups.

The term “independently” is used herein to indicate that the variable, which is independently applied, varies independently from application to application.

The term “lower alkyl” refers to methyl, ethyl or propyl

The term “lower alkoxy” refers to methoxy, ethoxy or propoxy.

In any of the embodiments described herein, the W, X, Y, Z, G, G′, R, R′, R″, Q1-Q4, A, and Rn can independently be covalently coupled to a linker and/or a linker to which is attached one or more PTM, ULM, ILM or ILM′ groups.

Exemplary MLMs

In certain additional embodiments, the MLM of the bifunctional compound comprises chemical moieties such as substituted imidazolines, substituted spiro-indolinones, substituted pyrrolidines, substituted piperidinones, substituted morpholinones, substituted pyrrolopyrimidines, substituted imidazolopyridines, substituted thiazoloimidazoline, substituted pyrrolopyrrolidinones, and substituted isoquinolinones.

In additional embodiments, the MLM comprises the core structures mentioned above with adjacent bis-aryl substitutions positioned as cis- or trans-configurations.

In still additional embodiments, the MLM comprises part of structural features as in RG7112, RG7388, SAR405838, AMG-232, AM-7209, DS-5272, MK-8242, and NVP-CGM-097, and analogs or derivatives thereof.

In certain preferred embodiments, MLM is a derivative of substituted imidazoline represented as Formula (A-1), or thiazoloimidazoline represented as Formula (A-2), or spiro indolinone represented as Formula (A-3), or pyrollidine represented as Formula (A-4), or piperidinone/morphinone represented as Formula (A-5), or isoquinolinone represented as Formula (A-6), or pyrollopyrimidine/imidazolopyridine represented as Formula (A-7), or pyrrolopyrrolidinone/imidazolopyrrolidinone represented as Formula (A-8).

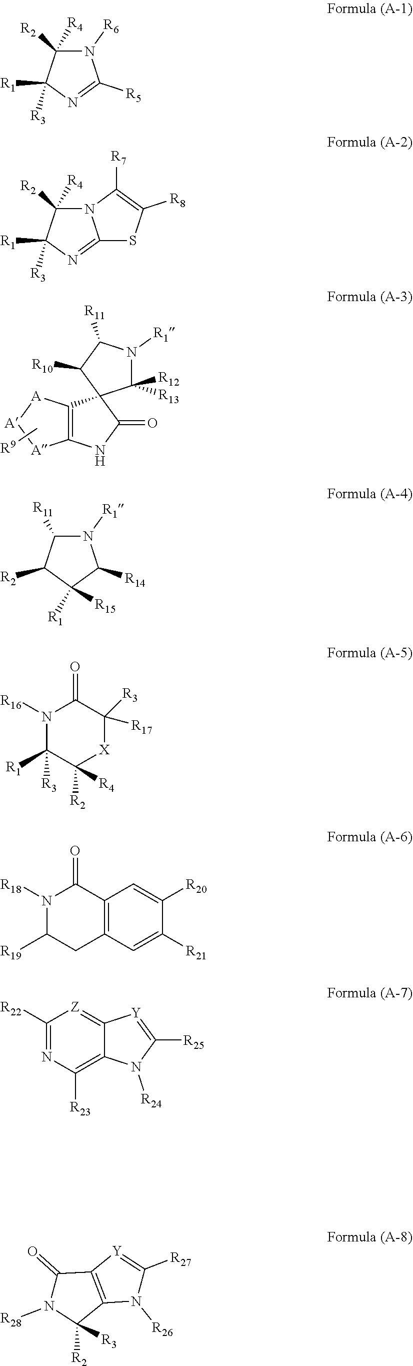

wherein above Formula (A-1) through Formula (A-8),
X of Formula (A-1) through Formula (A-8) is selected from the group consisting of carbon, oxygen, sulfur, sulfoxide, sulfone, and N—R^{a};
   R^{a} is independently H or an alkyl group with carbon number 1 to 6;
Y and Z of Formula (A-1) through Formula (A-8) are independently carbon or nitrogen;
A, A′ and A″ of Formula (A-1) through Formula (A-8) are independently selected from C, N, O or S, can also be one or two atoms forming a fused bicyclic ring, or a 6,5- and 5,5-fused aromatic bicyclic group;
R₁, R₂ of Formula (A-1) through Formula (A-8) are independently selected from the group consisting of an aryl or heteroaryl group, a heteroaryl group having one or two heteroatoms independently selected from sulfur or nitrogen, wherein the aryl or heteroaryl group can be mono-cyclic or bi-cyclic, or unsubstituted or substituted with one to three substituents independently selected from the group consisting of:
   halogen, —CN, C1 to C6 alkyl group, C3 to C6 cycloalkyl, —OH, alkoxy with 1 to 6 carbons, fluorine substituted alkoxy with 1 to 6 carbons, sulfoxide with 1 to 6 carbons, sulfone with 1 to 6 carbons, ketone with 2 to 6 carbons, amides with 2 to 6 carbons, and dialkyl amine with 2 to 6 carbons;
R₃, R₄ of Formula (A-1) through Formula (A-8) are independently selected from the group consisting of H, methyl and C₁ to C₆ alkyl;
R₅ of Formula (A-1) through Formula (A-8) is selected from the group consisting of an aryl or heteroaryl group, a heteroaryl group having one or two heteroatoms independently selected from sulfur or nitrogen, wherein the aryl or heteroaryl group can be mono-cyclic or bi-cyclic, or unsubstituted or substituted with one to three substituents independently selected from the group consisting of:
halogen, —CN, C₁ to C₆ alkyl group, C₃ to C₆ cycloalkyl, —OH, alkoxy with 1 to 6 carbons, fluorine substituted alkoxy with 1 to 6 carbons, sulfoxide with 1 to 6 carbons, sulfone with 1 to 6 carbons, ketone with 2 to 6 carbons, amides with 2 to 6 carbons, dialkyl amine with 2 to 6 carbons, alkyl ether (C₂ to C₆), alkyl ketone (C₃ to C₆), morpholinyl, alkyl ester (C₃ to C₆), alkyl cyanide (C₃ to C₆);
R₆ of Formula (A-1) through Formula (A-8) is H or —C(═O)R^{b}, wherein
   R^{b} of Formula (A-1) through Formula (A-8) is selected from the group consisting of alkyl, cycloalkyl, mono-, di- or tri-substituted aryl or heteroaryl, 4-morpholinyl, 1-(3-oxopiperazunyl), 1-piperidinyl, 4-N—R^{c}-morpholinyl, 4-R^{c}-1-piperidinyl, and 3-R^{c}-1-piperidinyl, wherein
   R^{c} of Formula (A-1) through Formula (A-8) is selected from the group consisting of alkyl, fluorine substituted alkyl, cyano alkyl, hydroxyl-substituted alkyl, cycloalkyl, alkoxyalkyl, amide alkyl, alkyl sulfone, alkyl sulfoxide, alkyl amide, aryl, heteroaryl, mono-, bis- and tri-substituted aryl or heteroaryl, CH2CH2R^{d}, and CH2CH2CH2R^{d}, wherein
   R^{d} of Formula (A-1) through Formula (A-8) is selected from the group consisting of alkoxy, alkyl sulfone, alkyl sulfoxide, N-substituted carboxamide, —NHC(O)-alkyl, —NH—SO₂-alkyl, aryl, substituted aryl, heteroaryl, substituted heteroaryl;
R₇ of Formula (A-1) through Formula (A-8) is selected from the group consisting of H, C1 to C₆ alkyl, cyclic alkyl, fluorine substituted alkyl, cyano substituted alkyl, 5- or 6-membered hetero aryl or aryl, substituted 5- or 6-membered hetero aryl or aryl;
R₈ of Formula (A-1) through Formula (A-8) is selected from the group consisting of —R^{e}—C(O)—R^{f}, —R^{e}-alkoxy, —R^{e}-aryl, —R^{e}-heteroaryl, and —R^{e}—C(O)—R^{f}—C(O)—R⁸, wherein:
   R^{e} of Formula (A-1) through Formula (A-8) is an alkylene with 1 to 6 carbons, or a bond;
   R^{f} of Formula (A-1) through Formula (A-8) is a substituted 4- to 7-membered heterocycle;
   R^{g} of Formula (A-1) through Formula (A-8) is selected from the group consisting of aryl, hetero aryl, substituted aryl or heteroaryl, and 4- to 7-membered heterocycle;
R₉ of Formula (A-1) through Formula (A-8) is selected from the group consisting of a mono-, bis- or tri-substituent on the fused bicyclic aromatic ring in Formula (A-3), wherein the substitutents are independently selected from the group consisting of halogen, alkene, alkyne, alkyl, unsubstituted or substituted with Cl or F;
R₁₀ of Formula (A-1) through Formula (A-8) is selected from the group consisting of an aryl or heteroaryl group, wherein the heteroaryl group can contain one or two heteroatoms as sulfur or nitrogen, aryl or heteroaryl group can be mono-cyclic or bi-cyclic, the aryl or heteroaryl group can be unsubstituted or substituted with one to three substituents, including a halogen, F, Cl, —CN, alkene, alkyne, C1 to C6 alkyl group, C1 to C6 cycloalkyl, —OH, alkoxy with 1 to 6 carbons, fluorine substituted alkoxy with 1 to 6 carbons, sulfoxide with 1 to 6 carbons, sulfone with 1 to 6 carbons, ketone with 2 to 6 carbons;
R₁₁ of Formula (A-1) through Formula (A-8) is —C(O)—N(R^{h})(Rⁱ), wherein R^{h} and Rⁱ are selected from groups consisting of the following:
   H; optionally substituted linear or branched C1 to C6 alkyl; alkoxy substituted alkyl; mono- and di-hydroxy substituted alkyl (e.g., a C3 to C6), sulfone substituted alkyl; optionally substituted aryl; optionally substituted heteraryl; mono-, bis- or tri-substituted aryl or heteroaryl; phenyl-4-carboxylic acid; substituted phenyl-4-carboxylic acid, alkyl carboxylic acid; optionally substituted heteroaryl carboxylic acid; alkyl carboxylic acid; fluorine substituted alkyl carboxylic acid; optionally substituted cycloalky, 3-hydroxycyclobutane, 4-hydroxycyclohehexane, aryl substituted cycloalkyl; heteroaryl substituted cycloalkyl; or Rh and Ri taken together form a ring;
R₁₂ and R₁₃ of Formula (A-1) through Formula (A-8) are independently selected from H, lower alkyl (C₁ to C₆), lower alkenyl (C₂ to C₆), lower alkynyl (C₂ to C₆), cycloalkyl (4, 5 and 6-membered ring), substituted cycloalkyl, cycloalkenyl, substituted cycloalkenyl, 5- and 6-membered aryl and heteroaryl, R₁₂ and R₁₃ can be connected to form a 5- and 6-membered ring with or without substitution on the ring;
R₁₄ of Formula (A-1) through Formula (A-8) is selected from the group consisting of alkyl, substituted alkyl, alkenyl, substituted alkenyl, aryl, substituted aryl, heteroaryl, substituted heteroaryl, heterocycle, substituted heterocycle, cycloalkyl, substituted cycloalkyl, cycloalkenyl and substituted cycloalkenyl;
R₁₅ of Formula (A-1) through Formula (A-8) is CN;
R₁₆ of Formula (A-1) through Formula (A-8) is selected from the group consisting of C₁₋₆ alkyl, C1-6 cycloalkyl, C2-6 alkenyl, C1-6 alkyl or C3-6 cycloalkyl with one or multiple hydrogens replaced by fluorine, alkyl or cycloalkyl with one CH₂ replaced by S(═O), —S, or —S(═O)₂, alkyl or cycloalkyl with terminal CH₃ replaced by S(═O)₂N(alkyl)(alkyl), —C(═O)N(alkyl)(alkyl), —N(alkyl)S(═O)₂(alkyl), —C(═O)2(alkyl), —O(alkyl), C₁₋₆ alkyl or alkyl-cycloalkyl with hydron replaced by hydroxyl group, a 3 to 7 membered cycloalkyl or heterocycloalkyl, optionally containing a —(C═O)— group, or a 5 to 6 membered aryl or heteroaryl group, which heterocycloalkyl or heteroaryl group can contain from one to three heteroatoms independently selected from O, N or S, and the cycloalkyl, heterocycloalkyl, aryl or heteroaryl group can be unsubstituted or substituted with from one to three substituents independently selected from halogen, C1-6 alkyl groups, hydroxylated C1-6 alkyl, C1-6 alkyl containing thioether, ether, sulfone, sulfoxide, fluorine substituted ether or cyano group;
R₁₇ of Formula (A-1) through Formula (A-8) is selected from the group consisting of (CH₂)nC(O)NR^{k}R^{l}, wherein R^{k} and R^{l} are independently selected from H, C1-6 alkyl, hydroxylated C1-6 alkyl, C1-6 alkoxy alkyl, C1-6 alkyl with one or multiple hydrogens replaced by fluorine, C₁₋₆ alkyl with one carbon replaced by S(O), S(O)(O), C1-6 alkoxyalkyl with one or multiple hydrogens replaced by fluorine, C1-6 alkyl with hydrogen replaced by a cyano group, 5 and 6 membered aryl or heteroaryl, alkyl aryl with alkyl group containing 1-6 carbons, and alkyl heteroaryl with alkyl group containing 1-6 carbons, wherein the aryl or heteroaryl group can be further substituted;
R₁₈ of Formula (A-1) through Formula (A-8) is selected from the group consisting of substituted aryl, heteroaryl, alkyl, cycloalkyl, the substitution is preferably —N(C1-4 alkylcycloalkyl), —N(C1-4 alkyl)alkyl-cycloalkyl, and —N(C1-4 alkyl)[(alkyl)-(heterocycle-substituted)-cycloalkyl];
Rig of Formula (A-1) through Formula (A-8) is selected from the group consisting of aryl, heteroaryl, bicyclic heteroaryl, and these aryl or heteroaryl groups can be substituted with halogen, C₁₋₆ alkyl, C₁₋₆ cycloalkyl, CF₃, F, CN, alkyne, alkyl sulfone, the halogen substitution can be mon-bis- or tri-substituted;
R₂₀ and R₂₁ of Formula (A-1) through Formula (A-8) are independently selected from C₁₋₆ alkyl, C₁₋₆ cycloalkyl, C₁₋₆ alkoxy, hydroxylated C₁₋₆ alkoxy, and fluorine substituted C₁₋₆ alkoxy, wherein R₂₀ and R₂₁ can further be connected to form a 5, 6 and 7-membered cyclic or heterocyclic ring, which can further be substituted;
R₂₂ of Formula (A-1) through Formula (A-8) is selected from the group consisting of H, C1-6 alkyl, C1-6 cycloalkyl, carboxylic acid, carboxylic acid ester, amide, reverse amide, sulfonamide, reverse sulfonamide, N-acyl urea, nitrogen-containing 5-membered heterocycle, the 5-membered heterocycles can be further substituted with C1-6 alkyl, alkoxy, fluorine-substituted alkyl, CN, and alkylsulfone;
R₂₃ of Formula (A-1) through Formula (A-8) is selected from aryl, heteroaryl, —O-aryl, —O— heteroaryl, —O-alkyl, —O-alkyl-cycloalkyl, —NH-alkyl, —NH-alkyl-cycloalkyl, —N(H)-aryl, —N(H)-heteroaryl, —N(alkyl)-aryl, —N(alkyl)-heteroaryl, the aryl or heteroaryl groups can be substituted with halogen, C1-6 alkyl, hydroxylated C1-6 alkyl, cycloalkyl, fluorine-substituted C₁₋₆ alkyl, CN, alkoxy, alkyl sulfone, amide and sulfonamide;
R₂₄ of Formula (A-1) through Formula (A-8) is selected from the group consisting of —CH2-(C1-6 alkyl), —CH2-cycloalkyl, —CH2-aryl, CH2-heteroaryl, where alkyl, cycloalkyl, aryl and heteroaryl can be substituted with halogen, alkoxy, hydroxylated alkyl, cyano-substituted alkyl, cycloalkyl and substituted cycloalkyl;
R₂₅ of Formula (A-1) through Formula (A-8) is selected from the group consisting of C1-6 alkyl, C1-6 alkyl-cycloalkyl, alkoxy-substituted alkyl, hydroxylated alkyl, aryl, heteroaryl, substituted aryl or heteroaryl, 5, 6, and 7-membered nitrogen-containing saturated heterocycles, 5,6-fused and 6,6-fused nitrogen-containing saturated heterocycles and these saturated heterocycles can be substituted with C1-6 alkyl, fluorine-substituted C1-6 alkyl, alkoxy, aryl and heteroaryl group;
R₂₆ of Formula (A-1) through Formula (A-8) is selected from the group consisting of C1-6 alkyl, C3-6 cycloalkyl, the alkyl or cycloalkyl can be substituted with —OH, alkoxy, fluorine-substituted alkoxy, fluorine-substituted alkyl, —NH₂, —NH-alkyl, NH—C(O)alkyl, —NH—S(O)₂-alkyl, and —S(O)₂-alkyl;
R₂₇ of Formula (A-1) through Formula (A-8) is selected from the group consisting of aryl, heteroaryl, bicyclic heteroaryl, wherein the aryl or heteroaryl groups can be substituted with C₁₋₆ alkyl, alkoxy, NH2, NH-alkyl, halogen, or —CN, and the substitution can be independently mono-, bis- and tri-substitution;
R₂₈ of Formula (A-1) through Formula (A-8) is selected from the group consisting of aryl, 5 and 6-membered heteroaryl, bicyclic heteroaryl, cycloalkyl, saturated heterocycle such as piperidine, piperidinone, tetrahydropyran, N-acyl-piperidine, wherein the cycloalkyl, saturated heterocycle, aryl or heteroaryl can be further substituted with —OH, alkoxy, mono-, bis- or tri-substitution including halogen, —CN, alkyl sulfone, and fluorine substituted alkyl groups; and
R_{1″} of Formula (A-1) through Formula (A-8) is selected from the group consisting of H, alkyl, aryl substituted alkyl, alkoxy substituted alkyl, cycloalkyl, aryl-substituted cycloalkyl, and alkoxy substituted cycloalkyl.

In certain embodiments, the heterocycles in R^{f} and R^{g} of Formula (A-1) through Formula (A-8) are substituted pyrrolidine, substituted piperidine, substituted piperizine.

More specifically, non-limiting examples of MLMs include those shown below as well as those ‘hybrid’ molecules that arise from the combination of 1 or more of the different features shown in the molecules below.

Using MLM in Formula A-1 through A-8, the following exemplary compounds can be prepared to target a particular protein for degradation, where ‘L” is a connector (i.e. a linker group), and “PTM” is a ligand binding to a target protein.

In certain embodiments, the description provides a bifunctional molecule comprising a structure selected from the group consisting of:

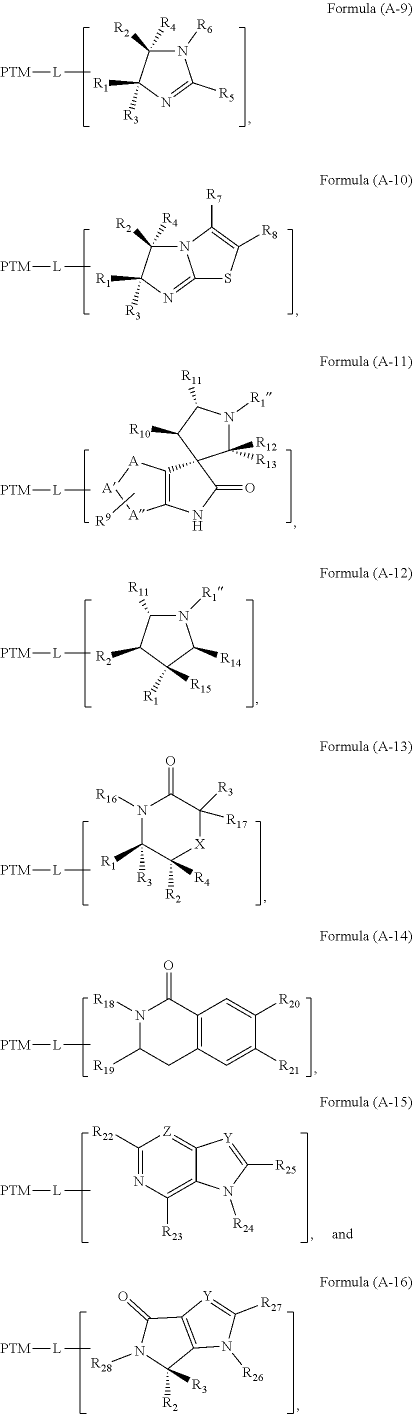

wherein X, R^{a}, Y, Z, A, A′, A″, R₁, R₂, R₃, R₄, R₅, R₆, R^{b}, R^{c}, R^{d}, R^{e}, R^{f}, R^{g}, R₉, R₁₀, R₁₁, R₁₂, R₁₃, R₁₄, R₁₅, R₁₆, R₁₇, R^{k}, R^{l}, R₁₈, R₁₉, R₂₀, R₂₁, R₂₂, R₂₃, R₂₄, R₂₅, R₂₆, R₂₇, R₂₈, and R_{1″} are as defined herein with regard to Formulas (A-1) through (A-8).

In certain embodiments, the description provides bifunctional or chimeric molecules with the structure: PTM-L-MLM, wherein PTM is a protein target binding moiety coupled to an MLM by L, wherein L is a bond (i.e., absent) or a chemical linker. In certain embodiments, the MLM has a structure selected from the group consisting of A-1-1, A-1-2, A-1-3, and A-1-4:

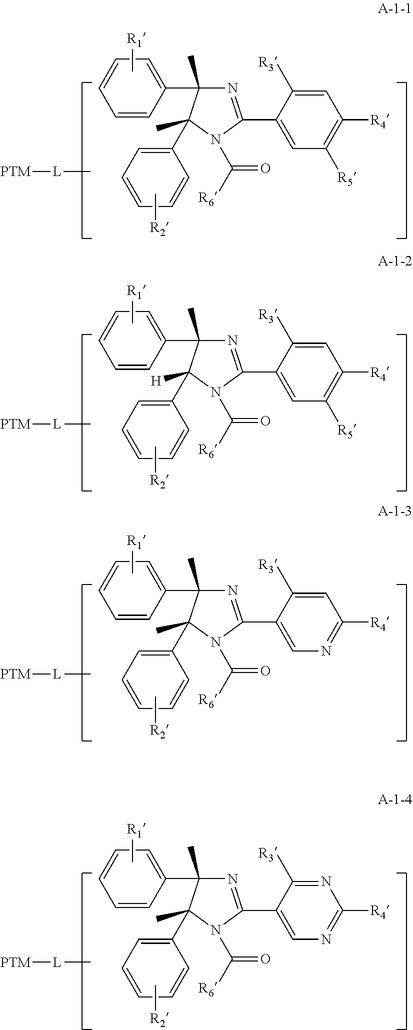

wherein:
R1′ and R2′ of Formulas A-1-1 through A-1-4 (i.e., A-1-1, A-1-2, A-1-3, and A-1-4) are independently selected from the group consisting of F, Cl, Br, I, acetylene, CN, CF₃ and NO₂;
R3′ is selected from the group consisting of —OCH₃, —OCH₂CH₃, —OCH₂CH₂F, —OCH₂CH₂OCH₃, and —OCH(CH₃)2;
R4′ of Formulas A-1-1 through A-1-4 is selected from the group consisting of H, halogen, —CH₃, —CF₃, —OCH₃, —C(CH₃)₃, —CH(CH₃)₂, -cyclopropyl, —CN, —C(CH₃)₂OH, —C(CH₃)₂OCH₂CH₃, —C(CH₃)₂CH₂OH, —C(CH₃)₂CH₂OCH₂CH₃, —C(CH₃)₂CH₂OCH₂CH₂OH, —C(CH₃)₂CH₂OCH₂CH₃, —C(CH₃)₂CN, —C(CH₃)₂C(O)CH₃, —C(CH₃)₂C(O)NHCH₃, —C(CH₃)₂C(O)N(CH₃)₂, —SCH₃, —SCH₂CH₃, —S(O)₂CH₃, —S(O₂)CH₂CH₃, —NHC(CH₃)₃, —N(CH₃)₂, pyrrolidinyl, and 4-morpholinyl;
R5′ of Formulas A-1-1 through A-1-4 is selected from the group consisting of halogen, -cyclopropyl, —S(O)₂CH₃, —S(O)₂CH₂CH_{3,1}-pyrrolidinyl, —NH₂, —N(CH₃)₂, and —NHC(CH₃)₃; and
R6′ of Formulas A-1-1 through A-1-4 is selected from the structures presented below where the linker connection point is indicated as
Beside R6′ as the point for linker attachment, R4′ can also serve as the linker attachment position. In the case that R4′ is the linker connection site, linker will be connected to the terminal atom of R4′ groups shown above.

In certain embodiments, the linker connection position of Formulas A-1-1 through A-1-4 is at least one of R4′ or R6′ or both.

In certain embodiments, R6′ of Formulas A-1-1 through A-1-4 is independently selected from the group consisting of H,

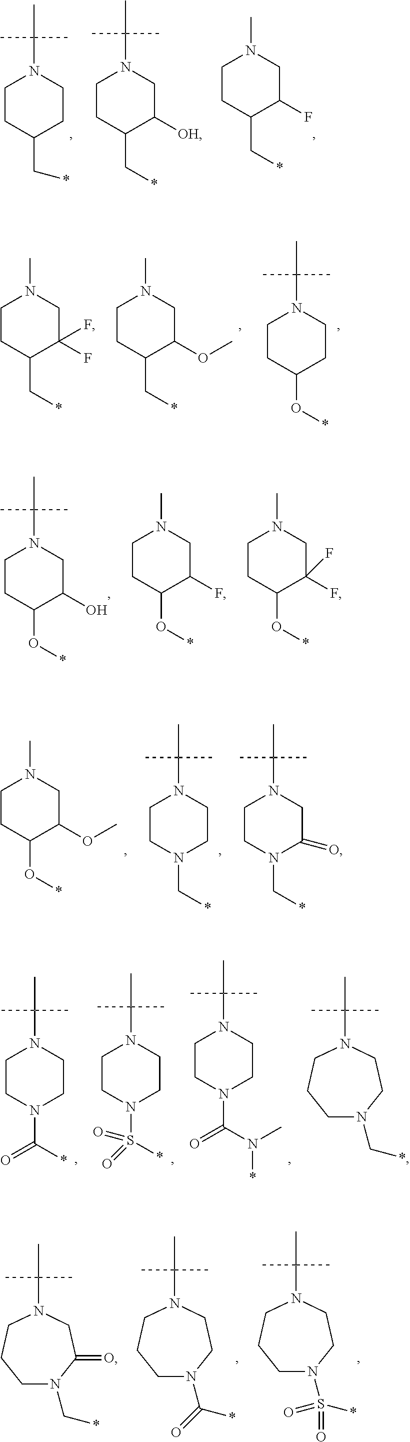

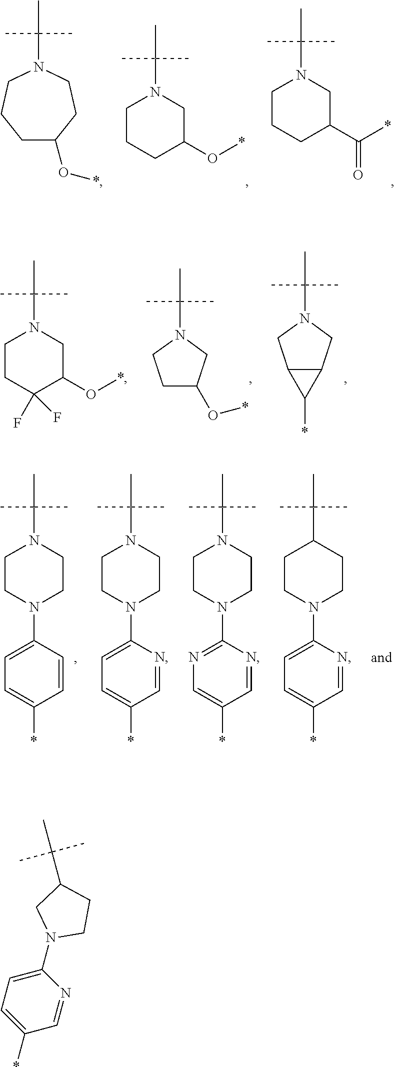

wherein indicates the point of attachment of the linker.

In certain embodiments, the linker of Formula A-4-1 through A-4-6 is attached to at least one of R1′, R2′, R3′, R4′, R5′, R6′, or a combination thereof.

In certain embodiments, the description provides bifunctional or chimeric molecules with the structure: PTM-L-MLM, wherein PTM is a protein target binding moiety coupled to an MLM by L, wherein L is a bond (i.e., absent) or a chemical linker. In certain embodiments, the MLM has a structure selected from the group consisting of A-4-1, A-4-2, A-4-3, A-4-4, A-4-5, and A-4-6:

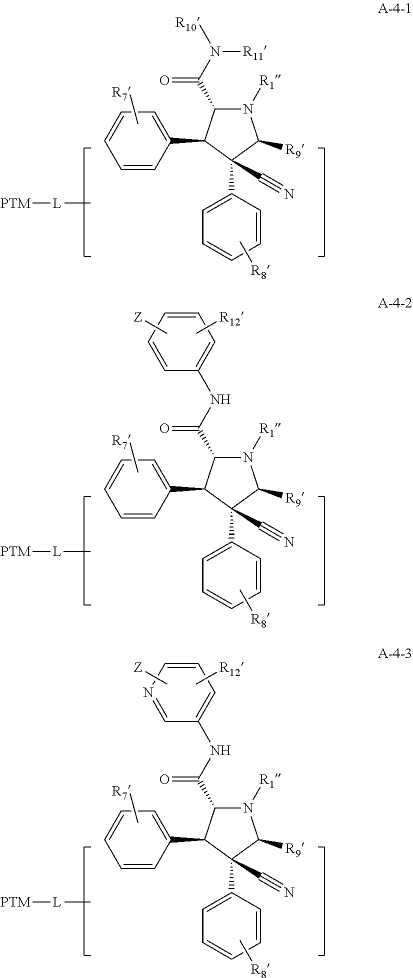

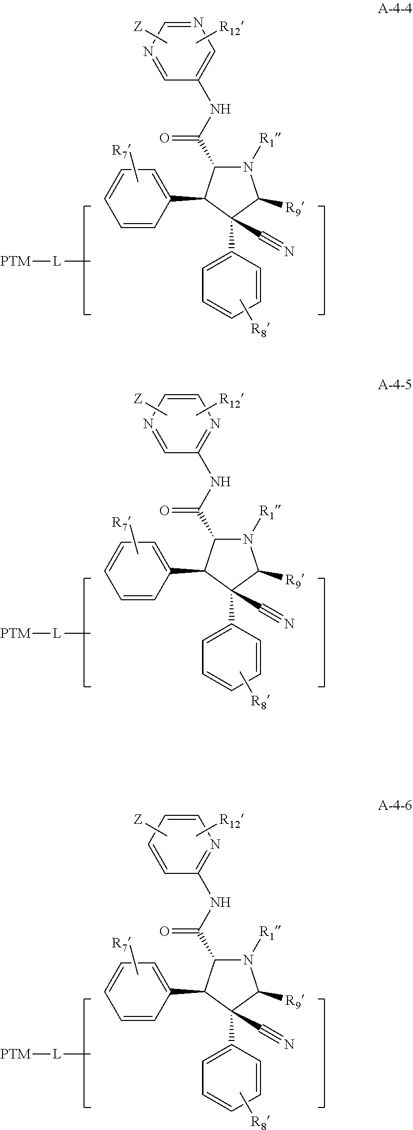

wherein:
R7′ of Formula A-4-1 through A-4-6 (i.e., A-4-1, A-4-2, A-4-3, A-4-4, A-4-5, and A-4-6) is one or more (e.g., 1, 2, 3, or 4) halogens;
R8′ of Formula A-4-1 through A-4-6 is one or more groups (e.g., 1, 2, 3, or 4 groups) selected from the group consisting of H, —F, —Cl, —Br, —I, —CN, —NO₂, ethylnyl, cyclopropyl, methyl, ethyl, isopropyl, vinyl, methoxy, ethoxy, isopropoxy, —OH, other C1-6 alkyl, other C1-6 alkenyl, and C1-6 alkynyl, mono-, di- or tri-substituted;
R9′ of Formula A-4-1 through A-4-6 is selected from the group consisting of alkyl, substituted alkyl, alkenyl, substituted alkenyl, alkynyl, substituted alkynyl, aryl, substituted aryl, hetero aryl, substituted heteroaryl, cycloalkyl, substituted cycloalkyl, alkenyl, and substituted cycloalkenyl;
Z of Formula A-4-1 through A-4-6 is selected from the group consisting of H, —OCH₃, —OCH₂CH₃, and halogen;
R10′ and R11′ of Formula A-4-1 through A-4-6 are each independently selected from the group consisting of H, (CH₂)ₙ—R′, (CH₂)ₙ—NR′R″, (CH₂)ₙ—NR′COR″, (CH₂)ₙ—NR′SO₂R″, (CH₂)ₙ—COOH, (CH₂)ₙ—COOR′, (CH)ₙ—CONR′R″, (CH₂)ₙ—OR′, (CH₂)ₙ—SR′, (CH₂)ₙ—SOR′, (CH₂)ₙ—CH(OH)—R′, (CH₂)ₙ—COR′, (CH₂)ₙ—SO₂R′, (CH₂)ₙ—SONR′R″, (CH₂)ₙ—SO₂NR′R″, (CH₂CH₂O)ₘ—(CH₂)ₙ—R′, (CH₂CH₂O)ₘ—(CH₂)ₙ—OH, (CH₂CH₂O)ₘ—(CH₂)ₙ—OR′, (CH₂CH₂O)ₘ—(CH₂)ₙ—NR′R″, (CH₂CH₂O)ₘ—(CH₂)ₙ—NR′COR″, (CH₂CH₂O)ₘ(CH₂)ₙ—NR′SO₂R″, (CH₂CH₂O)ₘ(CH₂)ₙ—COOH, (CH₂CH₂O)ₘ(CH₂)ₙ—COOR′, (CH₂CH₂O)ₘ—(CH₂)ₙ—CONR′R″, (CH₂CH₂O)ₘ—(CH₂)ₙ—SO₂R′, (CH₂CH₂O)ₘ—(CH₂)ₙ—COR′, (CH₂CH₂O)ₘ—(CH₂)ₙ—SONR′R″, (CH₂CH₂O)ₘ—(CH₂)ₙ—SO₂NR′R″, (CH₂)ₚ—(CH₂CH₂O)ₘ—(CH₂)ₙR′, (CH₂)p-(CH₂CH₂O)ₘ—(CH₂)ₙ—OH, (CH₂)ₚ—(CH₂CH₂O)ₘ—(CH₂)ₙ—OR′, (CH₂)ₚ—(CH₂CH₂O)ₘ—(CH₂)ₙ—NR′R″, (CH₂)ₚ—(CH₂CH₂O)ₘ—(CH₂)ₙ—NR′COR″, (CH₂)ₚ—(CH₂CH₂O)ₘ—(CH₂)ₙ—NR′SO₂R″, (CH₂)ₚ—(CH₂CH₂O)ₘ—(CH₂)ₙ—COOH, (CH₂)ₚ—(CH₂CH₂O)ₘ—(CH₂)ₙ—COOR′, (CH₂)ₚ—(CH₂CH₂O)ₘ—(CH₂)ₙ—CONR′R″, (CH₂)ₚ—(CH₂CH₂O)ₘ—(CH₂)ₙ—SO₂R′, (CH₂)ₚ—(CH₂CH₂O)ₘ—(CH₂)ₙ—COR′, (CH₂)ₚ—(CH₂CH₂O)ₘ—(CH₂)ₙ—SONR′R″, (CH₂)ₚ—(CH₂CH₂O)ₘ—(CH₂)ₙ—SO₂NR′R″, Aryl-(CH₂)ₙ—COOH, and heteroaryl-alkyl-CO-alkyl-NR′R″m, wherein the alkyl may be substituted with OR′, and heteroaryl-(CH₂)ₙ-heterocycle wherein the heterocycle may optionally be substituted with alkyl, hydroxyl, COOR′ and COR′; wherein R′ and R″ are selected from H, alkyl, alkyl substituted with halogen, hydroxyl, NH2, NH(alkyl), N(alkyl)₂, oxo, carboxy, cycloalkyl and heteroaryl;
m, n, and p are independently 0 to 6;
R12′ of Formula A-4-1 through A-4-6 is selected from the group consisting of —O-(alkyl), —O-(alkyl)-alkoxy, —C(O)-(alkyl), —C(OH)-alkyl-alkoxy, —C(O)—NH-(alkyl), —C(O)—N-(alkyl)₂, —S(O)-(alkyl), S(O)₂-(alkyl), —C(O)-(cyclic amine), and —O-aryl-(alkyl), —O-aryl-(alkoxy);
R1″ of Formula A-4-1 through A-4-6 is selected from the group consisting of alkyl, aryl substituted alkyl, alkoxy substituted alkyl, cycloalkyl, ary-substituted cycloalkyl, and alkoxy substituted cycloalkyl.

In any of the aspects or embodiments described herein, the alkyl, alkoxy or the like can be a lower alkyl or lower alkoxy.

In certain embodiments, the linker connection position of Formula A-4-1 through A-4-6 is at least one of Z, R8′, R9′, R10′, R11″, R12″, or R1″.

The method used to design chimeric molecules as presented in A-1-1 through A-1-4, A-4-1 through A-4-6 can be applied to MLM with formula A-2, A-3, A-5, A-6, A-7 and A-8, wherein the solvent exposed area in the MLM can be connected to linker “L” which will be attached to target protein ligand “PTM”, to construct bifunctional molecules of the present disclosure.

Exemplary MDM2 binding moieties include, but not limited, the following:

1. The HDM2/MDM2 inhibitors identified in Vassilev, et al., In vivo activation of the p53 pathway by small-molecule antagonists of MDM2, SCIENCE vol:303, pag:844-848 (2004), and Schneekloth, et al., Targeted intracellular protein degradation induced by a small molecule: En route to chemical proteomics, Bioorg. Med. Chem. Lett. 18 (2008) 5904-5908, including (or additionally) the compounds nutlin-3, nutlin-2, and nutlin-1 (derivatized) as described below, as well as all derivatives and analogs thereof:

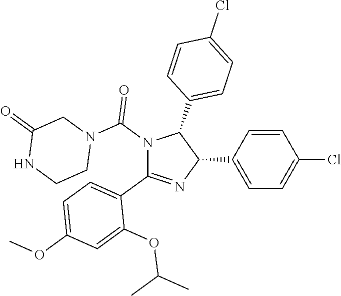 [SMILES: COc1ccc(C2=N[C@@H](c3ccc(Cl)cc3)[C@@H](c3ccc(Cl)cc3)N2C(=O)N2CCNC(=O)C2)c(OC(C)C)c1]

(derivatized where a linker group L or a -(L-MLM)group is attached, for example, at the methoxy group or as a hydroxyl group);

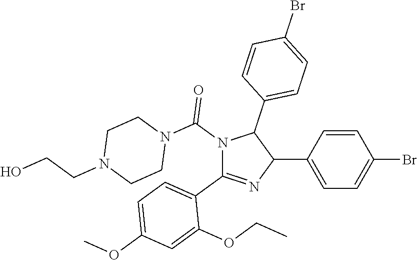 [SMILES: CCOc1cc(OC)ccc1C1=NC(c2ccc(Br)cc2)C(c2ccc(Br)cc2)N1C(=O)N1CCN(CCO)CC1]

(derivatized where a linker group L or a -(L-MLM) group is attached, for example, at the methoxy group or hydroxyl group);

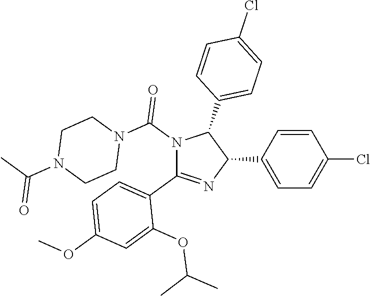 [SMILES: COc1ccc(C2=N[C@@H](c3ccc(Cl)cc3)[C@@H](c3ccc(Cl)cc3)N2C(=O)N2CCN(C(C)=O)CC2)c(OC(C)C)c1]

(derivatized where a linker group L or a -(L-MLM) group is attached, for example, via the methoxy group or as a hydroxyl group); and

2. Trans-4-Iodo-4′-Boranyl-Chalcone

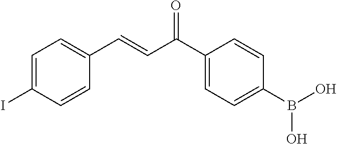 [SMILES: O=C(/C=C/c1ccc(I)cc1)c1ccc(B(O)O)cc1]

(derivatized where a linker group Lora a linker group L or a-(L-MLM) group is attached, for example, via a hydroxy group).

Exemplary CLMs

Neo-Imide Compounds

In one aspect the description provides compounds useful for binding and/or inhibiting cereblon. In certain embodiments, the compound is selected from the group consisting of chemical structures:

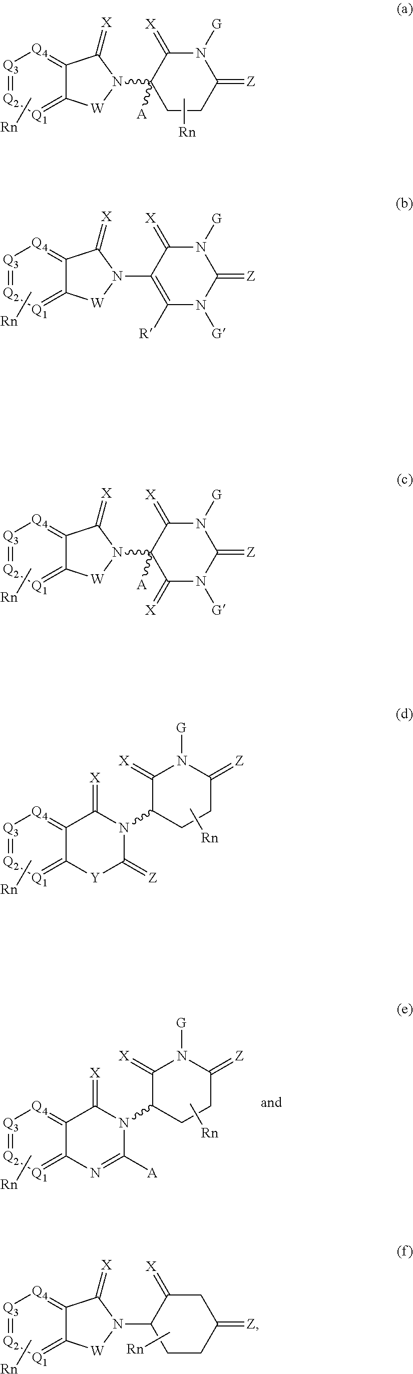

wherein:
W of Formulas (a) through (f) is independently selected from the group CH₂, CHR, C═O, SO₂, NH, and N-alkyl;
X of Formulas (a) through (f) is independently selected from the group absent, O, S and CH₂;
Y of Formulas (a) through (f) is independently selected from the group CH₂, —C═CR′, NH, N-alkyl, N-aryl, N-hetaryl, N-cycloalkyl, N-heterocyclyl, O, and S;
Z of Formulas (a) through (f) is independently selected from the group absent, O, S, or CH₂ except that both X and Z cannot be absent or CH₂;
G and G′ of Formulas (a) through (f) are independently selected from the group H, optionally substituted linear or branched alkyl, OH, R′OCOOR, R′OCONRR″, CH₂-heterocyclyl optionally substituted with R′, and benzyl optionally substituted with R′;
Q1-Q4 of Formulas (a) through (f) represent a carbon C substituted with a group independently selected from H, R, N or N-oxide;
A of Formulas (a) through (f) is independently selected from the group H, optionally substituted linear or branched alkyl, cycloalkyl, Cl and F;
R of Formulas (a) through (f) comprises, but is not limited to: —CONR′R″, —OR′, —NR′R″, —SR′, —SO₂R′, —SO₂NR′R″, —CR′R″—, —CR′NR′R″—, (—CR′O)_{n′}R″, optionally substituted heterocyclyl, optionally substituted -aryl, optionally substituted-heteroaryl, unsubstituted or substituted linear or branched alkyl, optionally substituted-cycloalkyl, optionally substituted-heterocyclyl, —P(O)(OR′)R″, —P(O)R′R″, —OP(O)(OR′)R″, —OP(O)R′R″, —Cl, —F, —Br, —I, —CF₃, —CN, —NR′SO₂NR′R″, —NR′CONR′R″, —CONR′COR″, —NR′C(═N—CN)NR′R″, —C(═N—CN)NR′R″, —NR′C(═N—CN)R″, —NR′C(═C—NO₂)NR′R″, —SO₂NR′COR″, —NO₂, —CO₂R′, —C(C═N—OR′)R″, —CR′═CR′R″, —CCR′, —S(C═O)(C═N—R′)R″, —SF₅ and —OCF₃, wherein at least one R is a functional group or atom independently selected from, for example, O, OH, N, NH, NH₂, C1-C6 alkyl, C1-C6 alkoxy, -alkyl-aryl (e.g., an -alkyl-aryl comprising at least one of C1-C6 alkyl, C4-C7 aryl, or a combination thereof), aryl (e.g., C5-C7 aryl), amine, amide, or carboxy);
R′ and R″ of Formulas (a) through (f) are independently selected from a bond, H, optionally substituted linear or branched alkyl, optionally substituted cycloalkyl, optionally substituted aryl, optionally substituted heteroaryl, optionally substituted heterocyclic, —C(═O)R, optionally substituted heterocyclyl;
n and n′ of Formulas (a) through (f) are independently an integer from 1-10 (e.g. 1-4, 1, 2, 3, 4, 5, 6, 7, 8, 9, or 10);
[symbol] of Formulas (a) through (f) represents a bond that may be stereospecific ((R) or (S)) or non-stereospecific.

Exemplary CLMs

In any of the compounds described herein, the CLM comprises a chemical structure selected from the group:

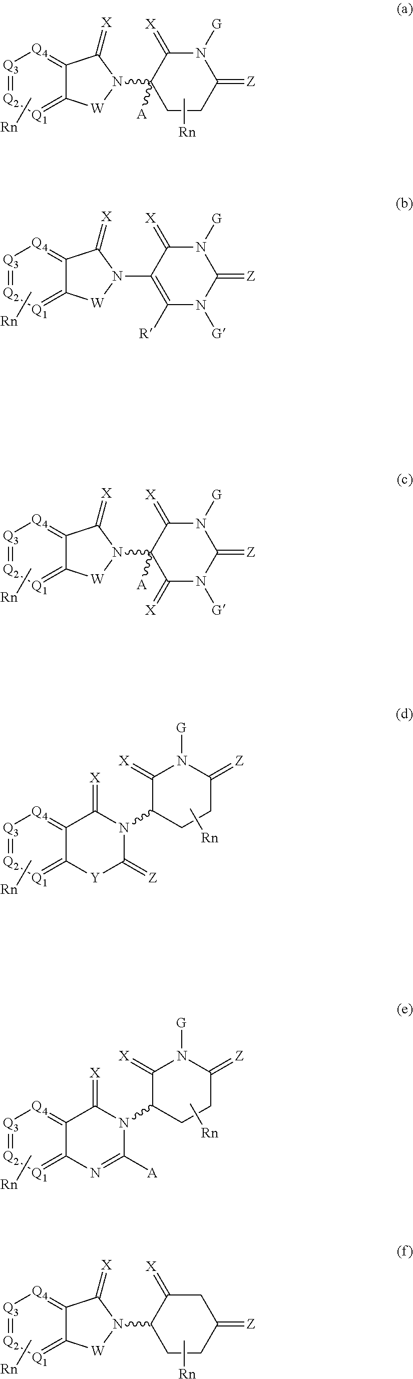

wherein:
W of Formulas (a) through (f) is independently selected from the group CH₂, CHR, C═O, SO₂, NH, and N-alkyl;
X of Formulas (a) through (f) is independently selected from the group absent, O, S and CH₂;
Y of Formulas (a) through (f) is independently selected from the group CH₂, —C═CR′, NH, N-alkyl, N-aryl, N-hetaryl, N-cycloalkyl, N-heterocyclyl, O, and S;
Z of Formulas (a) through (f) is independently selected from the group absent, O, S, and CH₂, except that both X and Z cannot be CH₂ or absent;
G and G′ of Formulas (a) through (f) are independently selected from the group H, optionally substituted linear or branched alkyl, OH, R′OCOOR, R′OCONRR″, CH₂-heterocyclyl optionally substituted with R and benzyl optionally substituted with R′;
Q1-Q4 of Formulas (a) through (f) represent a carbon C substituted with a group independently selected from H, R, N or N-oxide;
A of Formulas (a) through (f) is independently selected from the group H, alkyl (linear, branched, optionally substituted), cycloalkyl, Cl and F;
R of Formulas (a) through (f) comprises, but is not limited to: —CONR′R″, —OR′, —NR′R″, —SR′, —SO2R′, —SO2NR′R″, —CR′R″—, —CR′NR′R″—, (—CR′O)_{n′}R″, optionally substituted heterocyclyl, optionally substituted-aryl, optionally substituted-heteraryl, unsubstituted or substituted linear or branched alkyl, optionally substituted-cycloalkyl, optionally substituted-heterocyclyl, —P(O)(OR′)R″, —P(O)R′R″, —OP(O)(OR′)R″, —OP(O)R′R″, —Cl, —F, —Br, —I, —CF3, —CN, —NR′SO2NR′R″, —NR′CONR′R″, —CONR′COR″, —NR′C(═N—CN)NR′R″, —C(═N—CN)NR′R″, —NR′C(═N—CN)R″, —NR′C(═C—NO2)NR′R″, —SO2NR′COR″, —NO2, —CO2R′, —C(C═N—OR′)R″, —CR′═CR′R″, —CCR′, —S(C═O)(C═N—R′)R″, —SF5 and —OCF3, wherein at least one R (e.g., at least one of O, OH, N, NH, NH₂, C1-C6 alkyl, C1-C6 alkoxy, -alkyl-aryl (e.g., an -alkyl-aryl comprising at least one of C1-C6 alkyl, C4-C7 aryl, or a combination thereof), aryl (e.g., C5-C7 aryl), amine, amide, or carboxy) is modified to be covalently joined to a PTM, a chemical linker group (L), a ULM, a CLM′ (e.g., CLM′ is an additional CLM that has the same or different structure as a first CLM), or a combination thereof;
R′ and R″ of Formulas (a) through (f) are independently selected from a H, optionally substituted alkyl, optionally substituted cycloalkyl, optionally substituted aryl, optionally substituted heteroaryl, optionally substituted heterocyclic, —C(═O)R, optionally substituted heterocyclyl;
n and n′ of Formulas (a) through (f) are independently an integer from 1-10 (e.g., 1-4, 1, 2, 3, 4, 5, 6, 7, 8, 9, or 10);
[symbol] of Formulas (a) through (f) represents a bond that may be stereospecific ((R) or (S)) or non-stereospecific.

In certain embodiments described herein, the CLM or ULM comprises a chemical structure selected from the group:

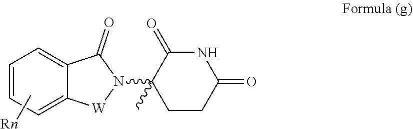

wherein:
W of Formula (g) is independently selected from the group CH₂, C═O, NH, and N-alkyl;
A is a H, methyl, or C1-C6 alkyl (linear, branched, optionally substituted);
R of Formula (g) is independently selected from a H, O, OH, N, NH, NH₂, methyl, optionally substituted alkyl (e.g., optionally substituted linear or branched C1-C6 alkyl), optionally substituted C1-C6 alkoxy, optionally substituted -alkyl-aryl (e.g., an -alkyl-aryl comprising at least one of C1-C6 alkyl, C4-C7 aryl, or a combination thereof), optionally substituted aryl (e.g., C5-C7 aryl), amine, amide, or carboxy, wherein at least one R (e.g., at least one of O, OH, N, NH, NH₂, C1-C6 alkyl, C1-C6 alkoxy, -alkyl-aryl (e.g., an -alkyl-aryl comprising at least one of C1-C6 alkyl, C4-C7 aryl, or a combination thereof), aryl (e.g., C5-C7 aryl), amine, amide, or carboxy) is modified to be covalently joined to a PTM, a chemical linker group (L), a ULM, CLM (or CLM′) or combination thereof;
n is an integer from 1 to 4; and
[symbol] of Formula (g) represents a bond that may be stereospecific ((R) or (S)) or non-stereospecific.

In any of the embodiments described herein, the W, X, Y, Z, G, G′, R, R′, R″, Q1-Q4, A, and R of Formulas (a) through (g) can independently be covalently coupled to a linker and/or a linker to which is attached one or more PTM, ULM, CLM or CLM′ groups.

In any of the aspects or embodiments described herein, Rn comprises from 1 to 4 independently selected functional groups or atoms, for example, O, OH, N, C1-C6 alkyl, C1-C6 alkoxy, -alkyl-aryl (e.g., an -alkyl-aryl comprising at least one of C1-C6 alkyl, C4-C7 aryl, or a combination thereof), aryl (e.g., C5-C7 aryl), amine, amide, or carboxy, and optionally, one of which is modified to be covalently joined to a PTM, a chemical linker group (L), a ULM, CLM (or CLM′) or combination thereof.

In any aspect or embodiment described herein, the R that is modified to be covalently joined to a PTM, a chemical linker group (L), a ULM, a CLM′, or a combination thereof is selected from O, OH, N, NH, NH₂, C1-C6 alkyl, C1-C6 alkoxy, -alkyl-aryl (e.g., an -alkyl-aryl comprising at least one of C1-C6 alkyl, C4-C7 aryl, or a combination thereof), aryl (e.g., C5-C7 aryl), amine, amide, or carboxy.

In some embodiments, the CLM is represented by the following structures with the dashed lines indicating linker attachment points:

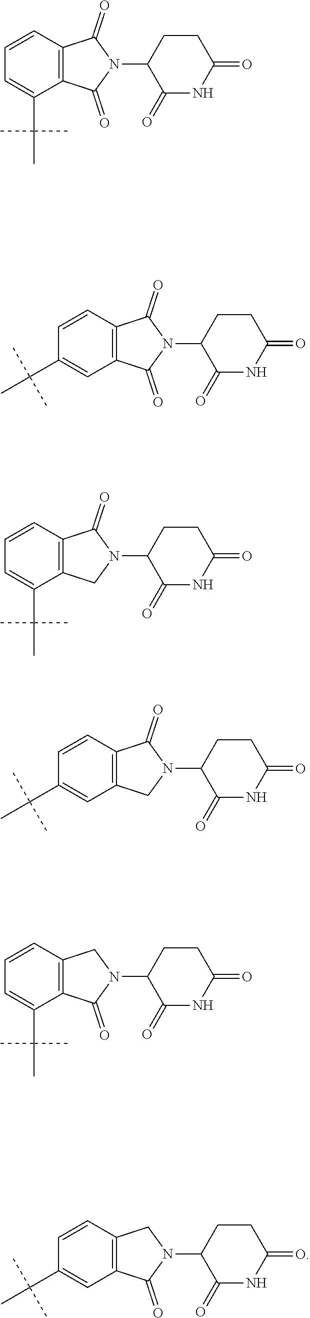

More specifically, non-limiting examples of CLMs include those shown below as well as those “hybrid” molecules that arise from the combination of 1 or more of the different features shown in the molecules below.

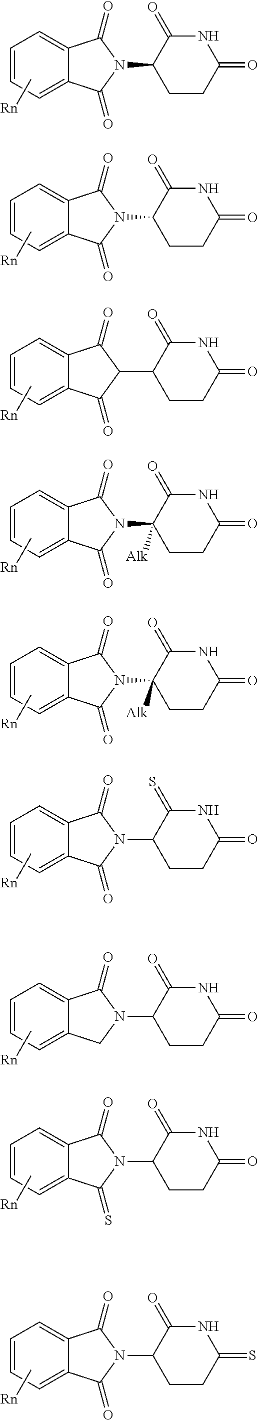

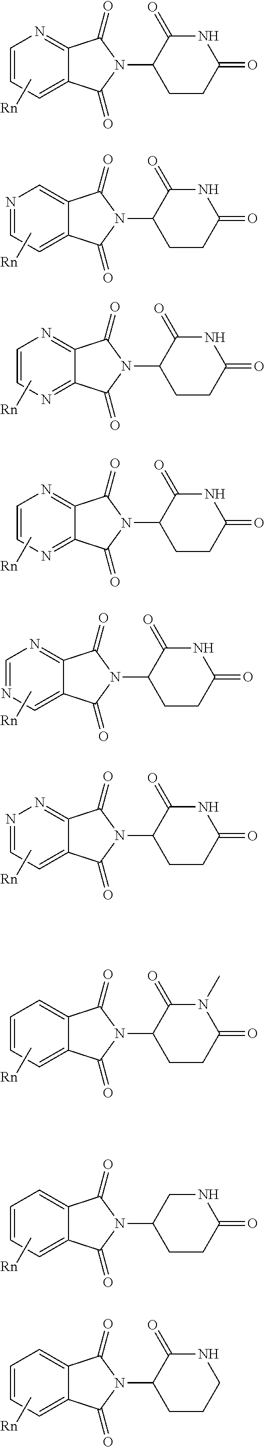

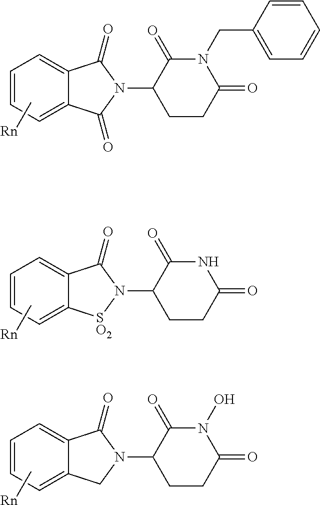

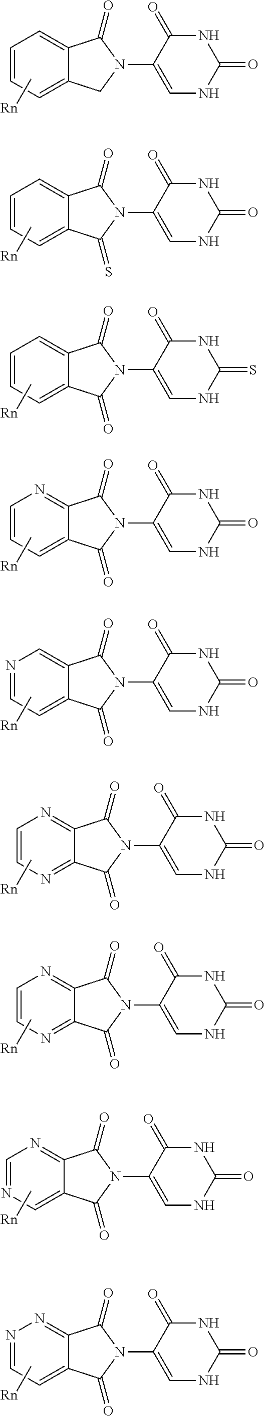

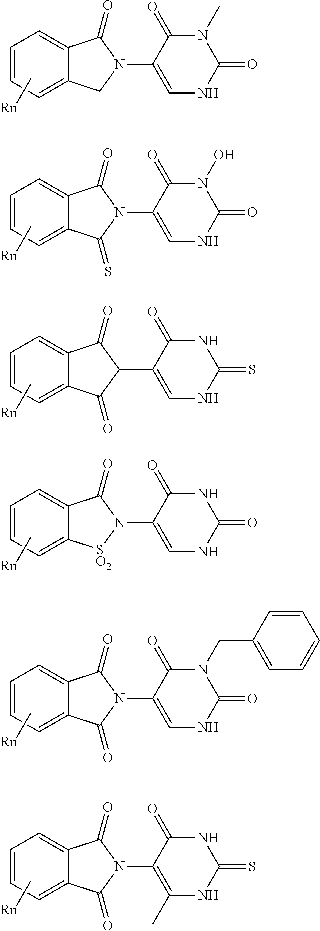

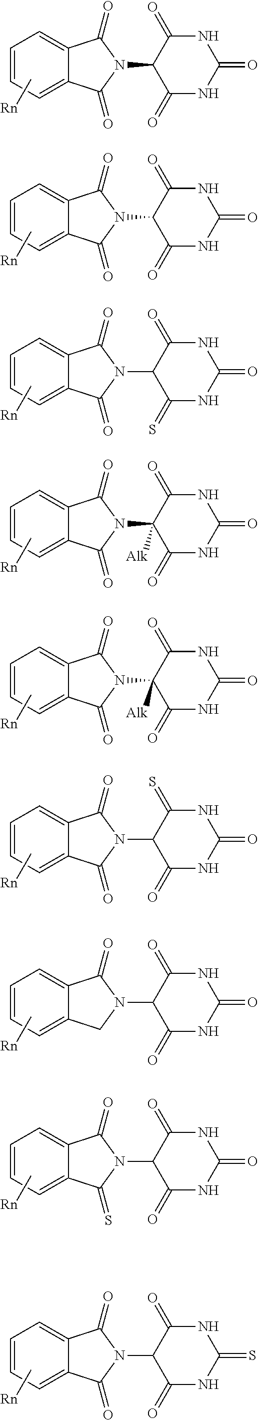

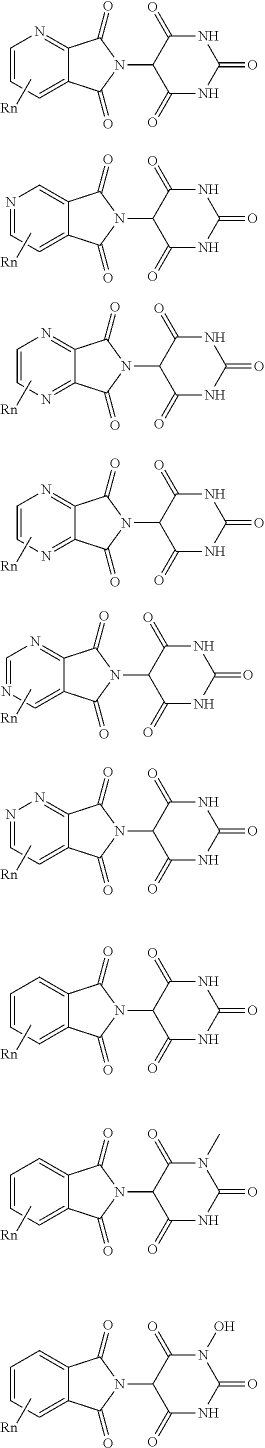

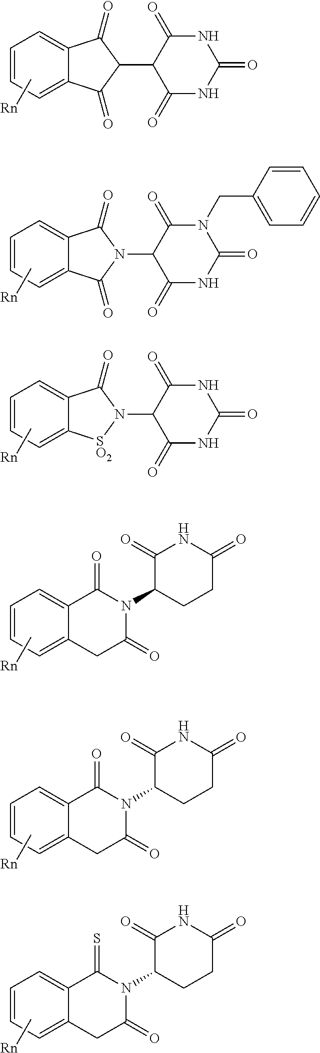

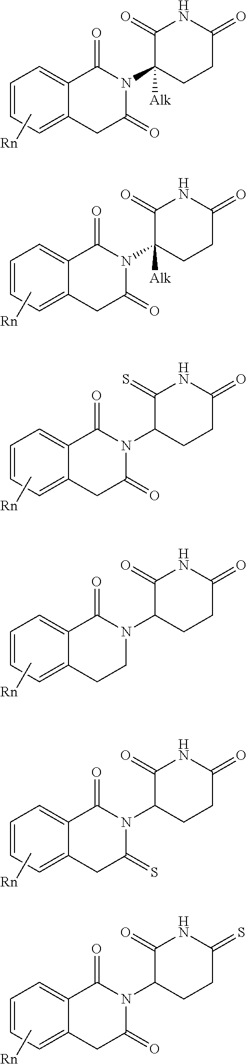

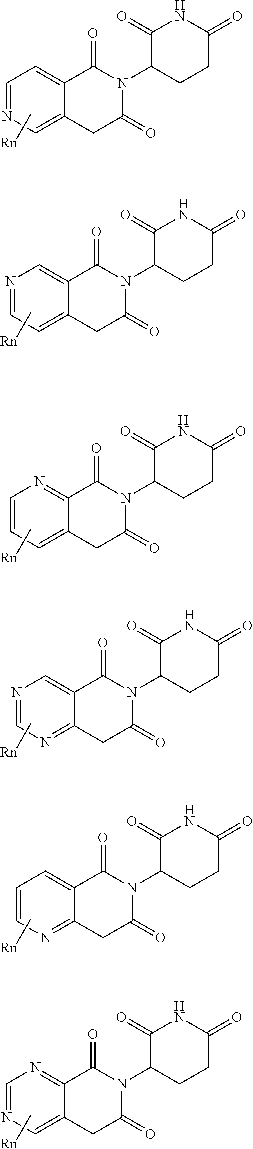

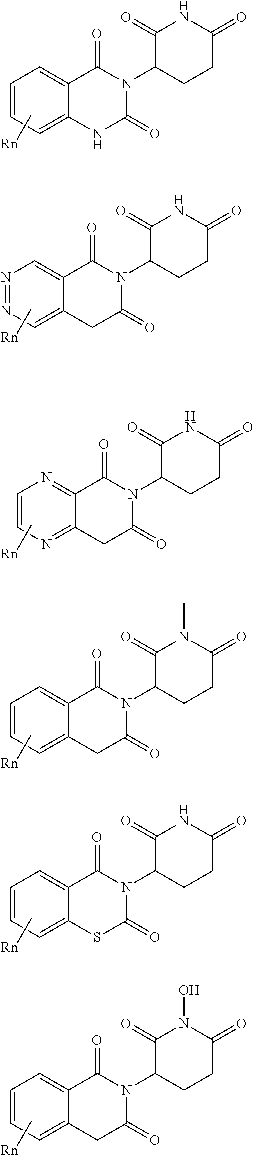

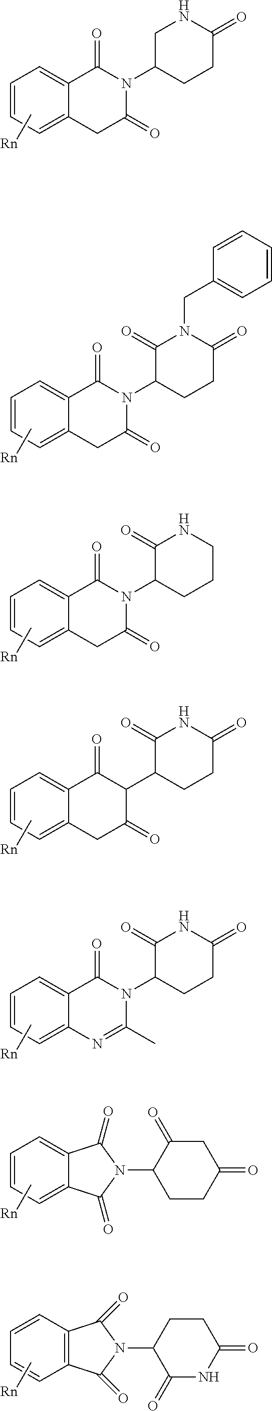

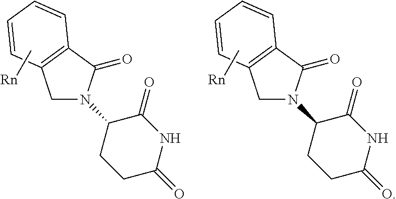

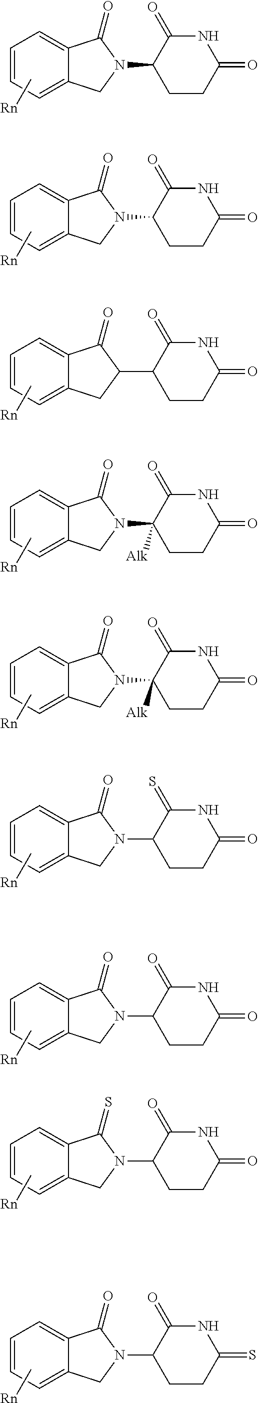

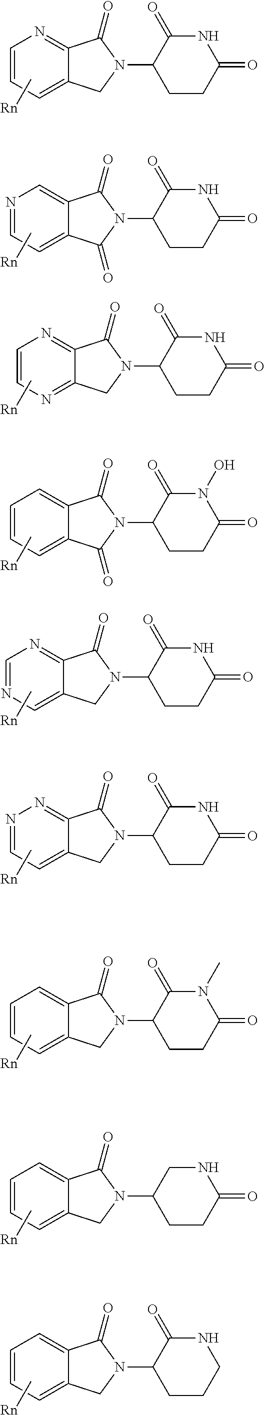

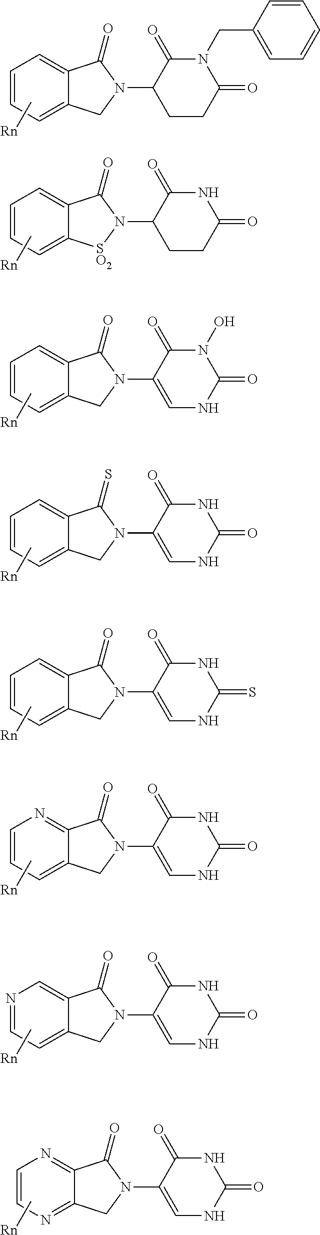

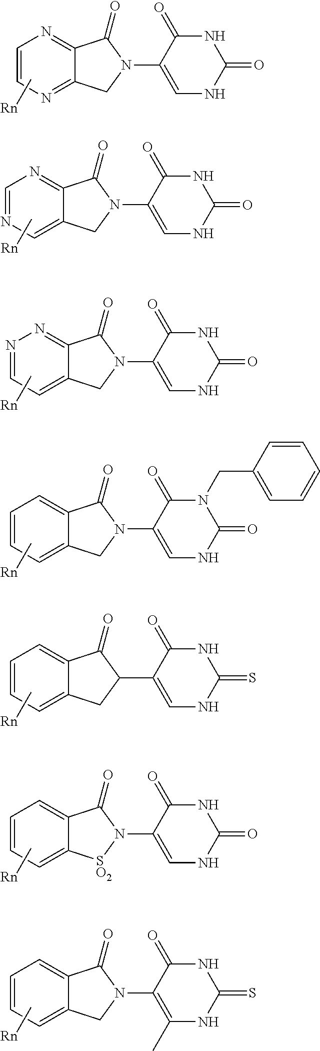

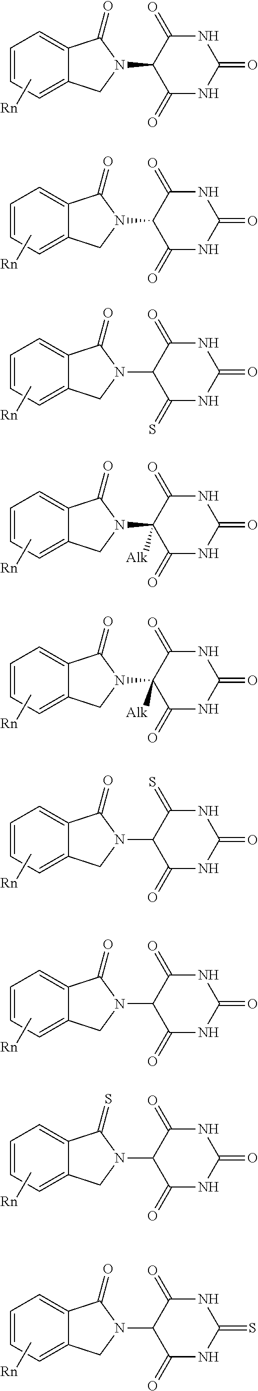

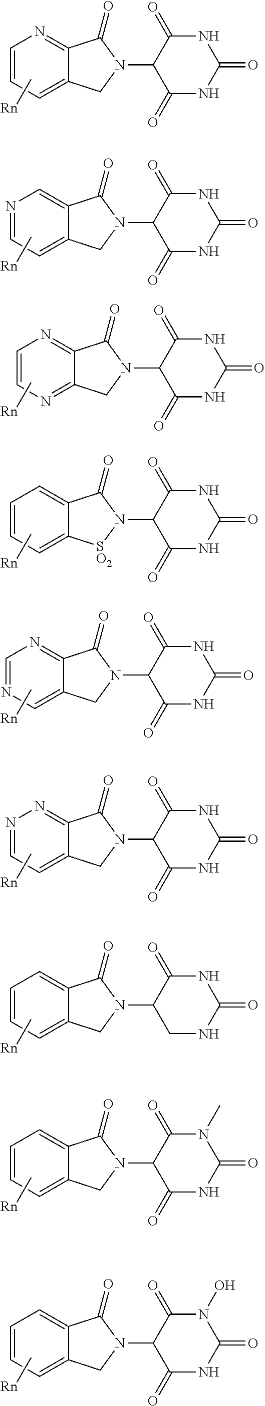

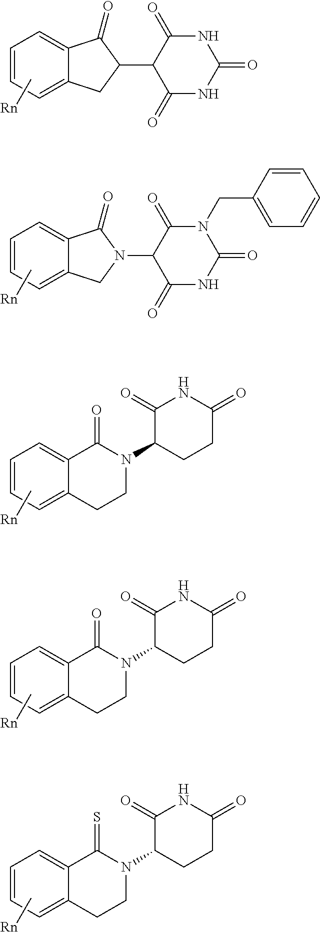

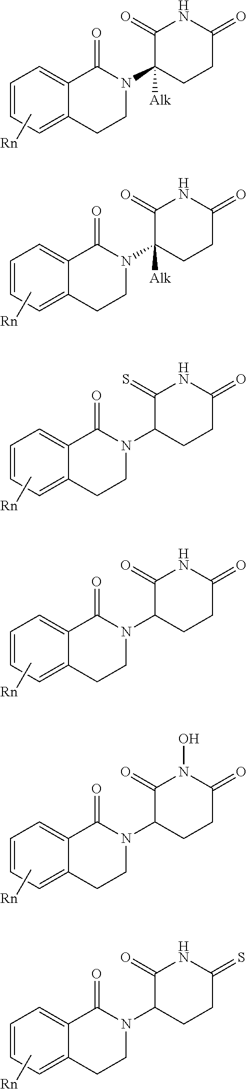

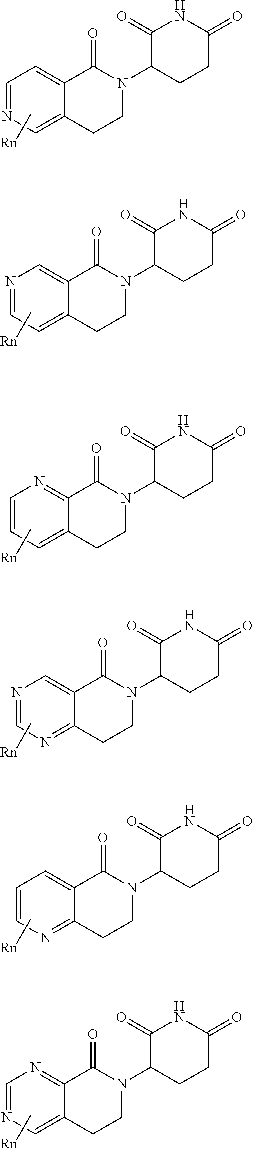

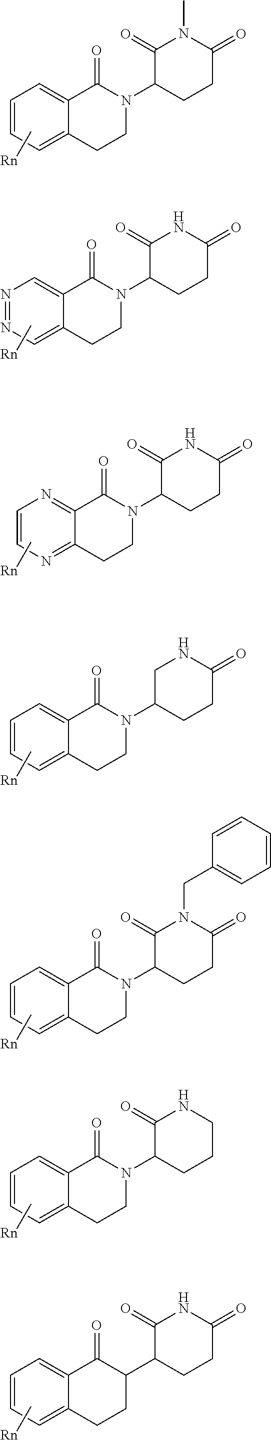

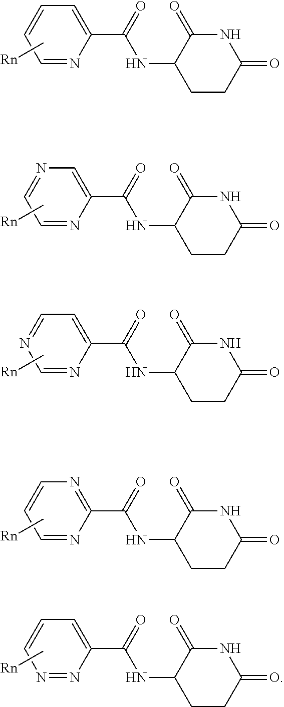

In any of the compounds described herein, the CLM comprises a chemical structure selected from the group:

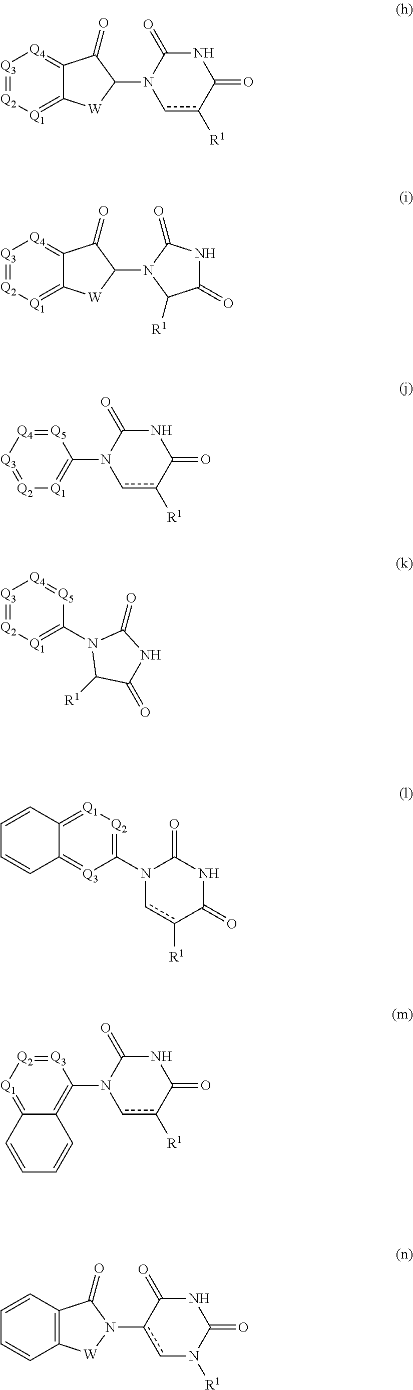

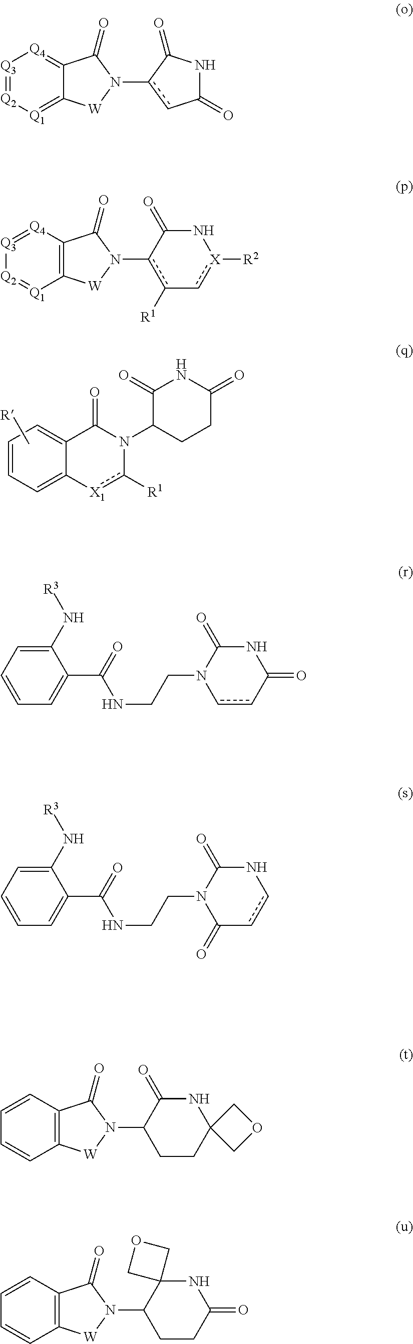

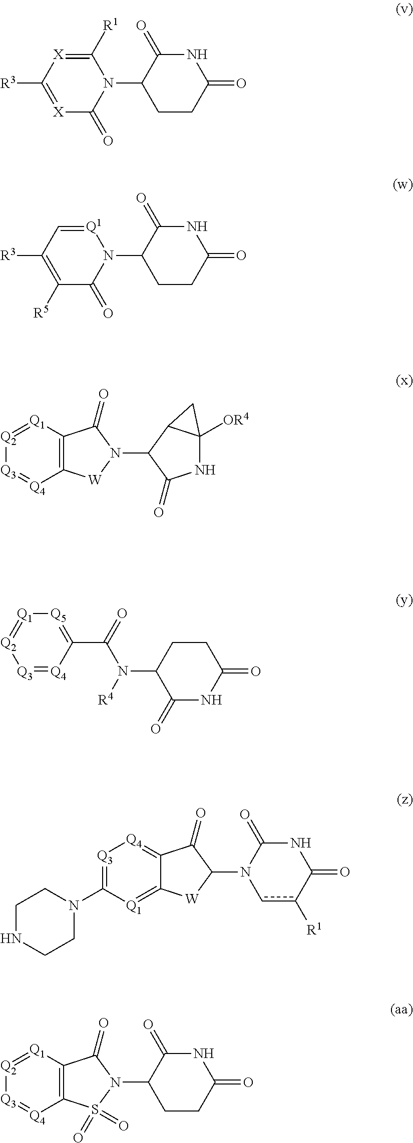

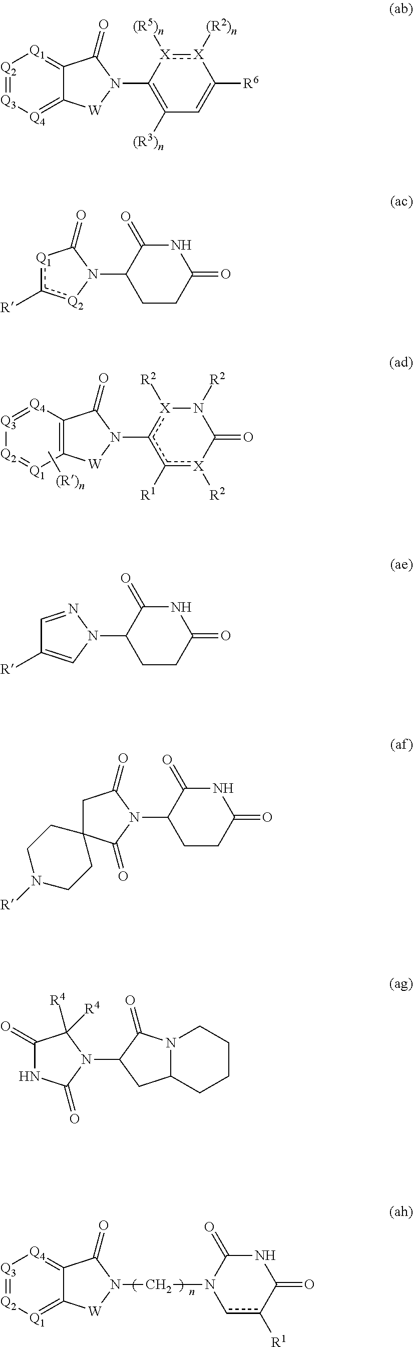

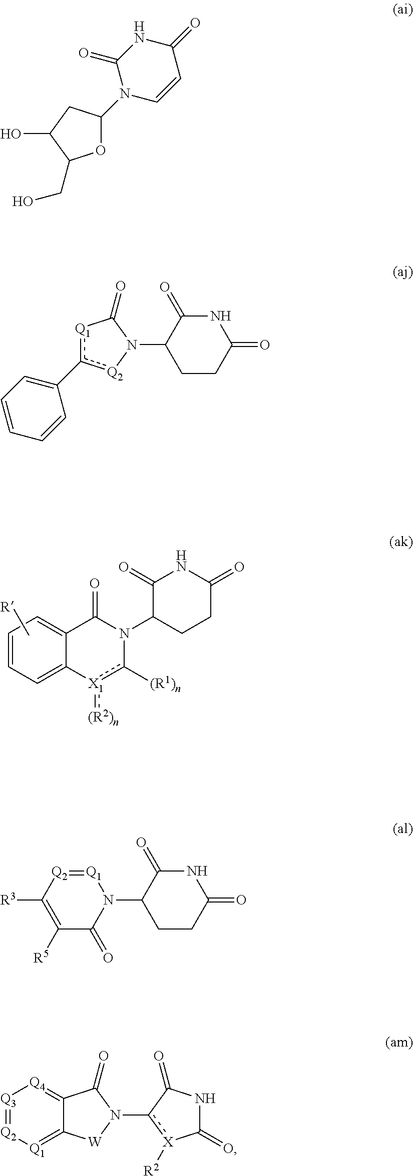

wherein:
W is independently selected from CH₂, CHR, C═O, SO₂, NH, and N-alkyl;
Q₁, Q₂, Q₃, Q₄, Q₅ are each independently represent a carbon C or N substituted with a group independently selected from R′, N or N-oxide;
R¹ is selected from absent, H, OH, CN, C1-C3 alkyl, C═O;
R² is selected from the group absent, H, OH, CN, C1-C3 alkyl, CHF₂, CF₃, CHO, C(═O)NH₂;
R³ is selected from H, alkyl (e.g., C1-C6 or C1-C3 alkyl), substituted alkyl (e.g., substituted C1-C6 or C1-C3 alkyl), alkoxy (e.g., C1-C6 or C1-C3 alkoxyl), substituted alkoxy (e.g., substituted C1-C6 or C1-C3 alkoxyl);
R⁴ is selected from H, alkyl, substituted alkyl;
R⁵ and R⁶ are each independently H, halogen, C(═O)R′, CN, OH, CF₃;
X is C, CH, C═O, or N;
X₁ is C═O, N, CH, or CH₂;
R′ is selected from H, halogen, amine, alkyl (e.g., C1-C3 alkyl), substituted alkyl (e.g., substituted C1-C3 alkyl), alkoxy (e.g., C1-C3 alkoxyl), substituted alkoxy (e.g., substituted C1-C3 alkoxyl), NR²R³, C(═O)OR², optionally substituted phenyl;
each n is independently an integer from 0 to 4;
[symbol] is a single or double bond; and
the CLM is covalently joined to a PTM, a chemical linker group (L), a ULM, CLM (or CLM′) or combination thereof.

In any aspect or embodiment described herein, the CLM or CLM′ is covalently joined to a PTM, a chemical linker group (L), a ULM, a CLM, a CLM′, or a combination thereof via an R group (such as, R, R¹, R², R³, R⁴ or R′), W, X, or a Q group (such as, Q₁, Q₂, Q₃, Q₄, or Q₅).

In any of the embodiments described herein, the CLM or CLM′ is covalently joined to a PTM, a chemical linker group (L), a ULM, a CLM, a CLM′, or a combination thereof via W, X, R, R¹, R², R³, R⁴, R⁵, R′, Q₁, Q₂, Q₃, Q₄, and Q₅.

In any of the embodiments described herein, the W, X, R¹, R², R³, R⁴, R′, Q₁, Q₂, Q₃, Q₄, and Q₅ can independently be covalently coupled to a linker and/or a linker to which is attached to one or more PTM, ULM, ULM′, CLM or CLM′ groups.

More specifically, non-limiting examples of CLMs include those shown below as well as “hybrid” molecules or compounds that arise from combining 1 or more features of the following compounds:

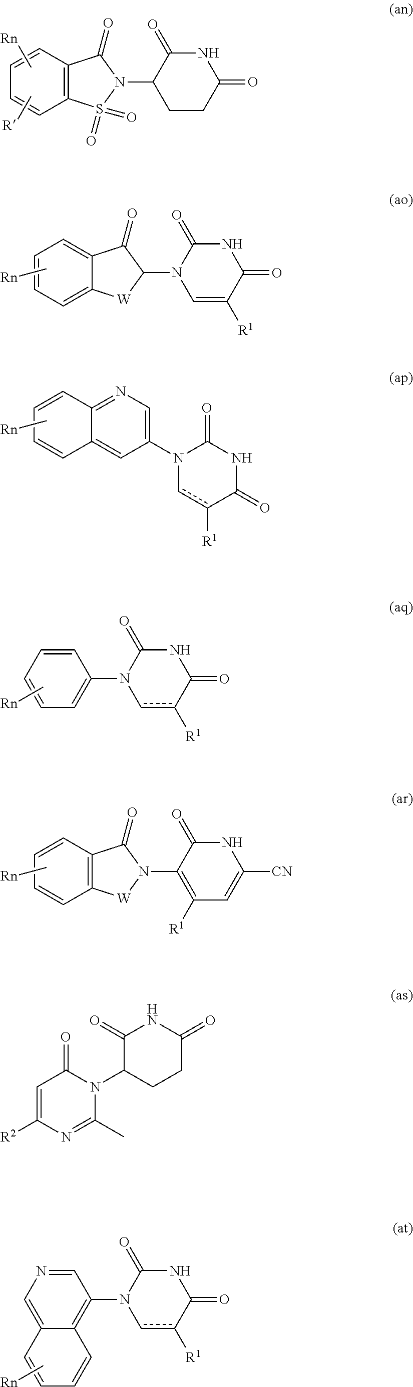

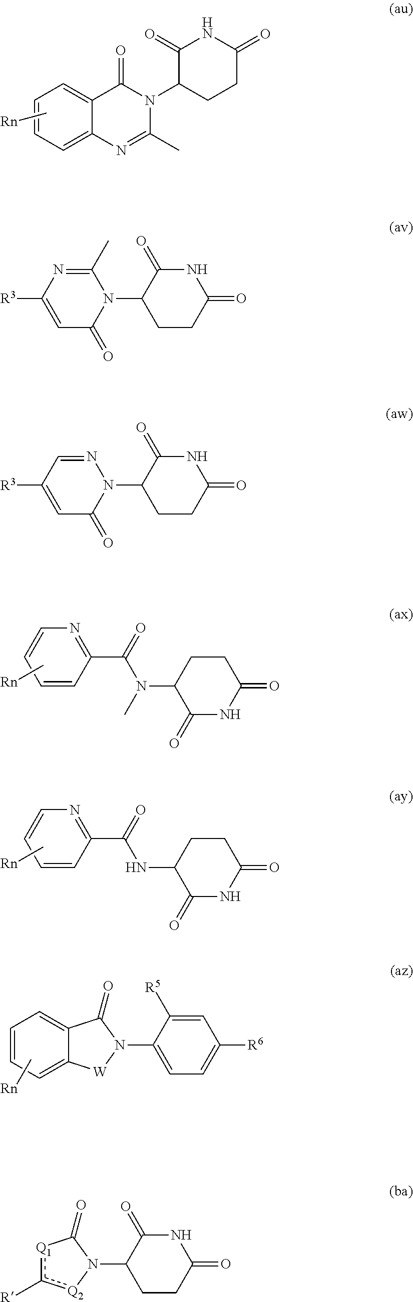

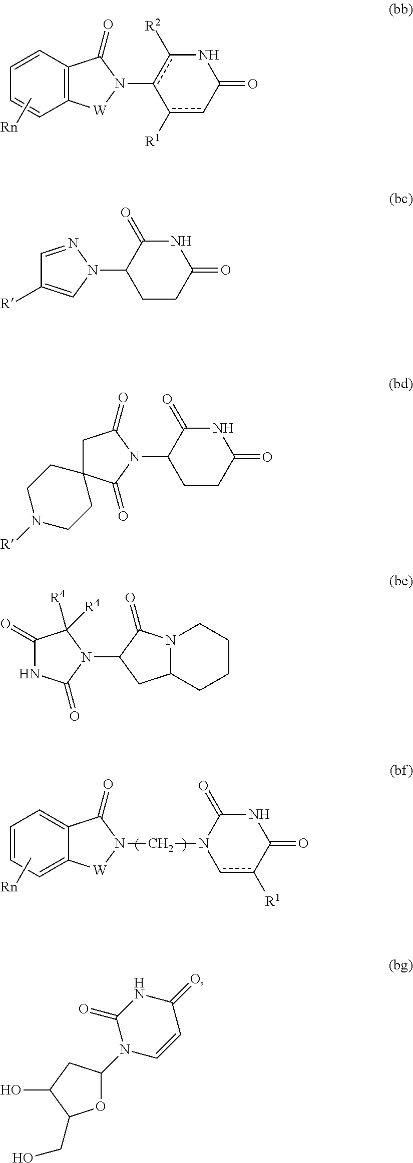

wherein:
W is independently selected from the group CH₂, CHR, C═O, SO₂, NH, and N-alkyl;
R¹ is selected from the group absent, H, CH, CN, C1-C3 alkyl;
R² is H or a C1-C3 alkyl;
R³ is selected from H, alkyl, substituted alkyl, alkoxy, substituted alkoxy;
R⁴ is methyl or ethyl;
R⁵ is H or halo;
R⁶ is H or halo;
R′ is H or an attachment point for a PTM, a PTM′, a chemical linker group (L), a ULM, a CLM, a CLM′,
Q₁ and Q₂ are each independently C or N substituted with a group independently selected from H or C1-C3 alkyl;
[symbol] is a single or double bond;
n is an integer from 1 to 4; and
R comprises is an atom (e.g., H) or functional group.

In any aspect or embodiment described herein, at least one R is modified to be covalently joined to a PTM, a chemical linker group (L), a ULM, a CLM′ (e.g., CLM′ is an additional CLM that has the same or different structure as a first CLM), or a combination thereof.

In any of the embodiments described herein, the W, R¹, R², Q₁, Q₂, Q₃, Q₄, and R can independently be covalently coupled to a linker and/or a linker to which is attached one or more PTM, ULM, ULM′, CLM or CLM′ groups.

In any of the embodiments described herein, the R¹, R², Q₁, Q₂, Q₃, Q₄, and R can independently be covalently coupled to a linker and/or a linker to which is attached one or more PTM, ULM, ULM′, CLM or CLM′ groups.

In any of the embodiments described herein, the Q₁, Q₂, Q₃, Q₄, and R can independently be covalently coupled to a linker and/or a linker to which is attached one or more PTM, ULM, ULM′, CLM or CLM′ groups.

In any aspect or embodiment described herein, R is modified to be covalently joined to the linker group (L), a PTM, a ULM, a second CLM having the same chemical structure as the CLM, a CLM′, a second linker, or any multiple or combination thereof.

In any aspect or embodiment described herein, the CLM is selected from:

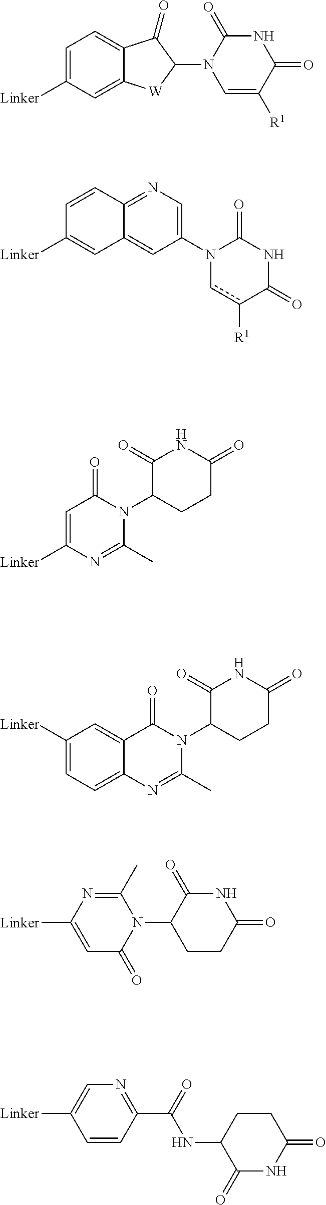

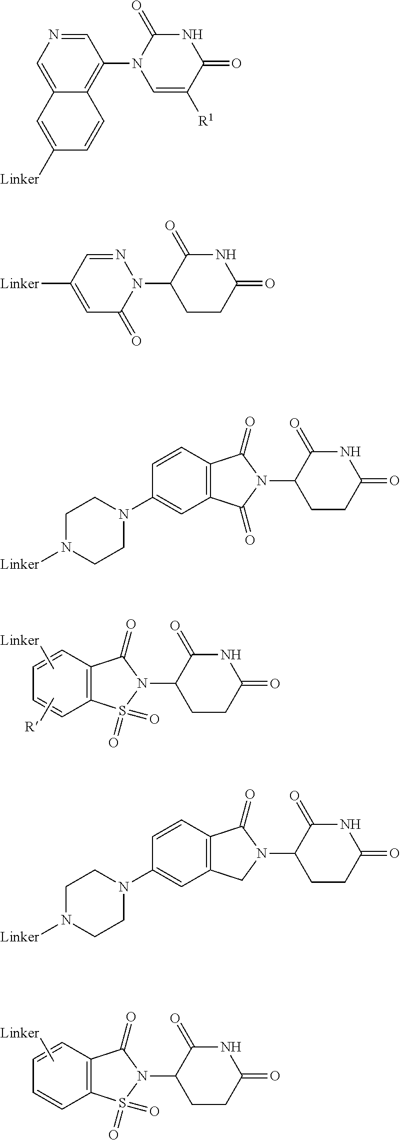

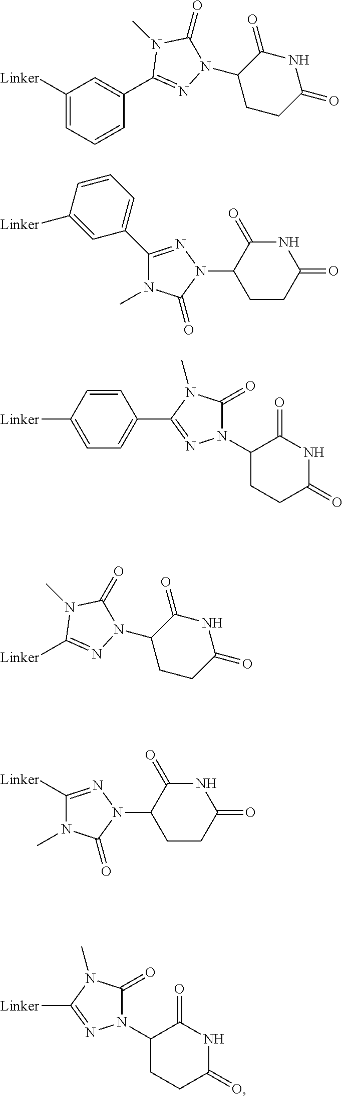

wherein R′ is a halogen and R¹ is as described in any aspect or embodiment described herein.

In certain cases, “CLM” can be imides that bind to cereblon E3 ligase. These imides and linker attachment point can be but not limited to the following structures:

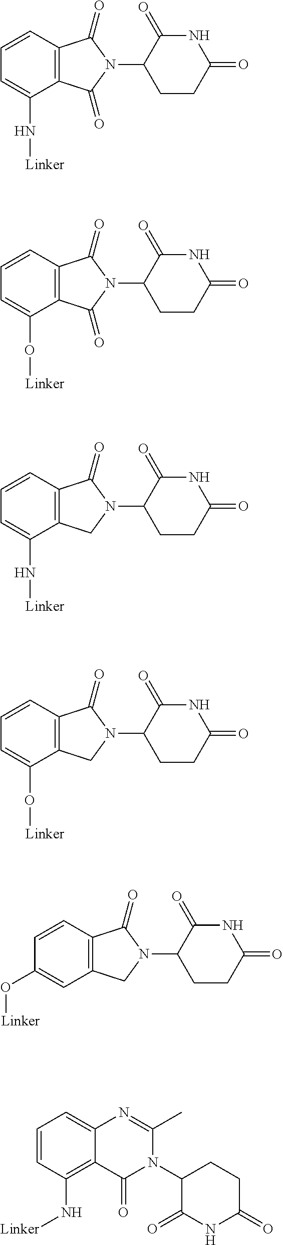

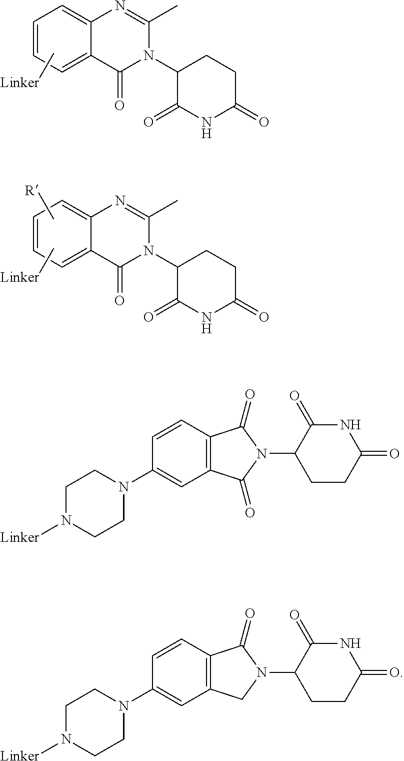

Exemplary VLMs

In certain embodiments of the compounds as described herein, ULM is VLM and comprises a chemical structure selected from the group ULM-a:

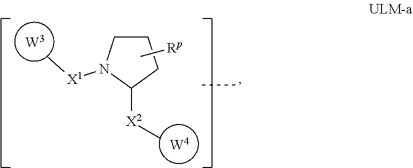

wherein:
a dashed line indicates the attachment of at least one PTM, another ULM or VLM or MLM or ILM or CLM (i.e., ULM′ or VLM′ or CLM′ or ILM′ or MLM′), or a chemical linker moiety coupling at least one PTM, a ULM′ or a VLM′ or a CLM′ or a ILM′ or a MLM′ to the other end of the linker;
X¹, X² of Formula ULM-a are each independently selected from the group of a bond, O, NR^{Y3}, CR^{Y3}R^{Y4}, C═O, C═S, SO, and SO₂;
R^{Y3}, R^{Y4} of Formula ULM-a are each independently selected from the group of H, linear or branched C₁₋₆ alkyl, optionally substituted by 1 or more halo, optionally substituted C₁₋₆ alkoxyl (e.g., optionally substituted by 0-3 R^{p} groups);
R^{P} of Formula ULM-a is 0, 1, 2, or 3 groups, each independently selected from the group H, halo, —OH, C₁₋₃ alkyl, C═O;
W³ of Formula ULM-a is selected from the group of an optionally substituted T, an optionally substituted -T-N(R^{1a}R^{1b})X³, optionally substituted -T-N(R^{1a}R^{1b}), optionally substituted -T-Aryl, an optionally substituted -T-Heteroaryl, an optionally substituted T-biheteroaryl, an optionally substituted -T-Heterocycle, an optionally substituted -T-biheterocycle, an optionally substituted —NR¹-T-Aryl, an optionally substituted —NR¹-T-Heteroaryl or an optionally substituted —NR¹-T-Heterocycle;
X³ of Formula ULM-a is C═O, R¹, R^{1a}, R^{1b};
each of R¹, R^{1a}, R^{1b} is independently selected from the group consisting of H, linear or branched C1-C6 alkyl group optionally substituted by 1 or more halo or —OH groups, R^{Y3}C═O, R^{Y3}C═S, R^{Y3}SO, R^{Y3}SO₂, N(R^{Y3}R^{Y4})C═O, N(R^{Y3}R^{Y4})C═S, N(R^{Y3}R^{Y4})SO, and N(R^{Y3}R^{Y4})SO₂;
T of Formula ULM-a is selected from the group of an optionally substituted alkyl, —(CH₂)ₙ— group, wherein each one of the methylene groups is optionally substituted with one or two substituents selected from the group of halogen, methyl, optionally substituted alkoxy, a linear or branched C1-C6 alkyl group optionally substituted by 1 or more halogen, C(O) NR¹R^{1a}, or NR¹R^{1a} or R¹ and R^{1a} are joined to form an optionally substituted heterocyclyl, or —OH groups or an amino acid side chain optionally substituted;
W⁴ of Formula ULM-a is an optionally substituted —NR1-T-Aryl wherein the aryl group may be optionally substituted with an optionally substituted 5-6 membered heteroaryl, an optionally substituted —NR1-T-Heteroaryl group or an optionally substituted —NR1-T-Heterocycle, where —NR1 is covalently bonded to X² and R¹ is H or CH₃, preferably H; and
n is 0 to 6, often 0, 1, 2, or 3, preferably 0 or 1.

In any of the embodiments described herein, T is selected from the group of an optionally substituted alkyl, —(CH₂)ₙ— group, wherein each one of the methylene groups is optionally substituted with one or two substituents selected from the group of halogen, methyl, optionally substituted alkoxy, a linear or branched C1-C6 alkyl group optionally substituted by 1 or more halogen, C(O) NR¹R^{1a}, or NR¹R^{1a} or R¹ and R^{1a} are joined to form an optionally substituted heterocycle, or —OH groups or an amino acid side chain optionally substituted; and n is 0 to 6, often 0, 1, 2, or 3, preferably 0 or 1.

In certain embodiments, W⁴ of Formula ULM-a is

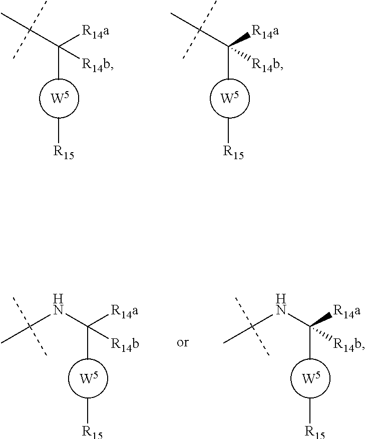

wherein: R₁₄ₐ, R_{14b}, are each independently selected from the group of H, haloalkyl, or optionally substituted alkyl, and W⁵ is optionally substituted.

In any of the embodiments, W⁵ of Formula ULM-a is selected from the group of an optionally substituted phenyl or an optionally substituted 5-10 membered heteroaryl,

R₁₅ of Formula ULM-a is selected from the group of H, halogen, CN, OH, NO₂, NR₁₄ₐR_{14b}, OR₁₄ₐ, CONR₁₄ₐR_{14b}, NR₁₄ₐCOR_{14b}, SO₂NR₁₄ₐR_{14b}, NR₁₄ₐSO₂R_{14b}, optionally substituted alkyl, optionally substituted haloalkyl, optionally substituted haloalkoxy, optionally substituted aryl, optionally substituted heteroaryl, optionally substituted cycloalkyl, or optionally substituted cycloheteroalkyl;

In additional embodiments, W⁴ substituents for use in the present disclosure also include specifically (and without limitation to the specific compound disclosed) the W⁴ substituents which are found in the identified compounds disclosed herein. Each of these W⁴ substituents may be used in conjunction with any number of W³ substituents which are also disclosed herein.

In certain additional embodiments, ULM-a, is optionally substituted by 0-3 R^{p} groups in the pyrrolidine moiety. Each R^{p} is independently H, halo, —OH, C1-3alkyl, C═O.

In any of the embodiments described herein, the W³, W⁴ of Formula ULM-a can independently be covalently coupled to a linker which is attached one or more PTM groups. and wherein the dashed line indicates the site of attachment of at least one PTM, another ULM (ULM′) or a chemical linker moiety coupling at least one PTM or a ULM′ or both to ULM.

In certain embodiments, ULM is VHL and is represented by the structure:

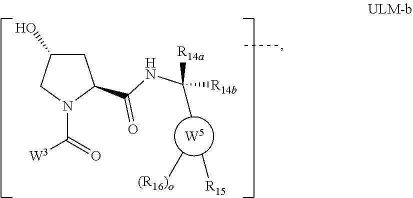

wherein:
W³ of Formula ULM-b is selected from the group of an optionally substituted aryl, optionally substituted heteroaryl, or

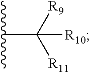

R₉ and R₁₀ of Formula ULM-b are independently hydrogen, optionally substituted alkyl, optionally substituted cycloalkyl, optionally substituted hydroxyalkyl, optionally substituted heteroaryl, or haloalkyl, or R₉, R₁₀, and the carbon atom to which they are attached form an optionally substituted cycloalkyl;
R₁₁ of Formula ULM-b is selected from the group of an optionally substituted heterocyclic, optionally substituted alkoxy, optionally substituted heteroaryl, optionally substituted aryl,

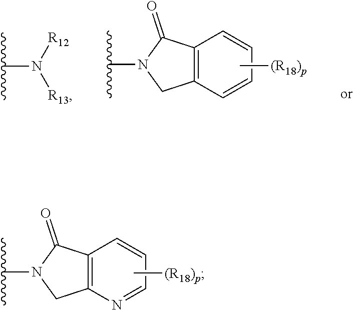

R₁₂ of Formula ULM-b is selected from the group of H or optionally substituted alkyl;
R₁₃ of Formula ULM-b is selected from the group of H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl;
R₁₄ₐ, R_{14b} of Formula ULM-b, are each independently selected from the group of H, haloalkyl, or optionally substituted alkyl;
W⁵ of Formula ULM-b is selected from the group of an optionally substituted phenyl or an optionally substituted 5-10 membered heteroaryl,
R₁₅ of Formula ULM-b is selected from the group of H, halogen, CN, OH, NO₂, NR₁₄ₐR_{14b}, OR₁₄ₐ, CONR₁₄ₐR_{14b}, NR₁₄ₐCOR_{14b}, SO₂NR₁₄ₐR_{14b}, NR₁₄ₐSO₂R_{14b}, optionally substituted alkyl, optionally substituted haloalkyl, optionally substituted haloalkoxy, optionally substituted aryl, optionally substituted heteroaryl, optionally substituted cycloalkyl, or optionally substituted cycloheteroalkyl;
each R₁₆ of Formula ULM-b is independently selected from the group of H, CN, halo, optionally substituted alkyl, optionally substituted haloalkyl, hydroxy, or optionally substituted haloalkoxy;
o of Formula ULM-b is 0, 1, 2, 3, or 4;
R₁₈ of Formula ULM-b is independently selected from the group of H, halo, optionally substituted alkoxy, cyano, optionally substituted alkyl, haloalkyl, haloalkoxy or a linker; and
p of Formula ULM-b is 0, 1, 2, 3, or 4, and wherein the dashed line indicates the site of attachment of at least one PTM, another ULM (ULM′) or a chemical linker moiety coupling at least one PTM or a ULM′ or both to ULM.

In certain embodiments, R₁₅ of Formula ULM-b is

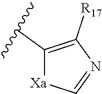

wherein R₁₇ is H, halo, optionally substituted C₃₋₆cycloalkyl, optionally substituted C₁₋₆alkyl, optionally substituted C₁₋₆alkenyl, and C₁₋₆haloalkyl; and Xa is S or O.

In certain embodiments, R₁₇ of Formula ULM-b is selected from the group methyl, ethyl, isopropyl, and cyclopropyl.

In certain additional embodiments, R₁₅ of Formula ULM-b is selected from the group consisting of:

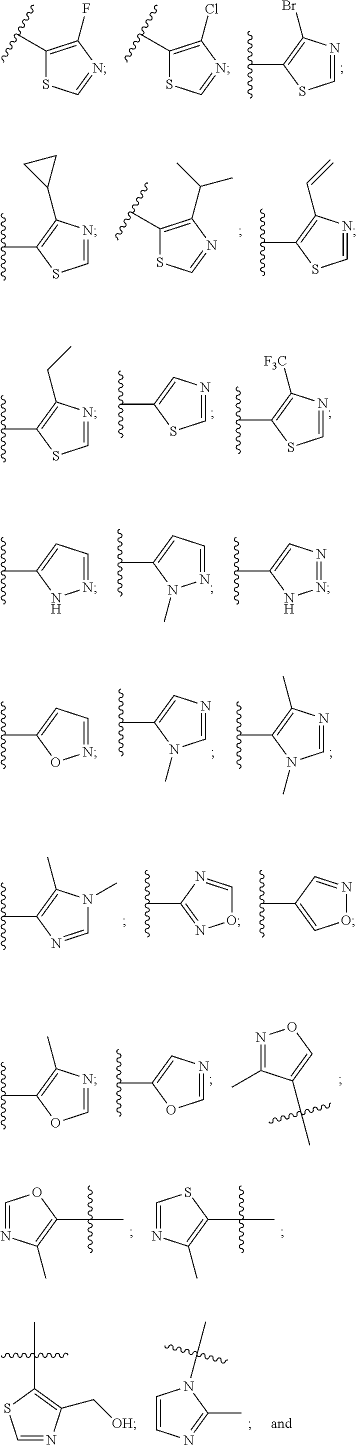

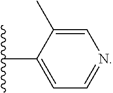 [SMILES: *c1ccncc1C]

In certain embodiments, R₁₁ of Formula ULM-b is selected from the group consisting of:

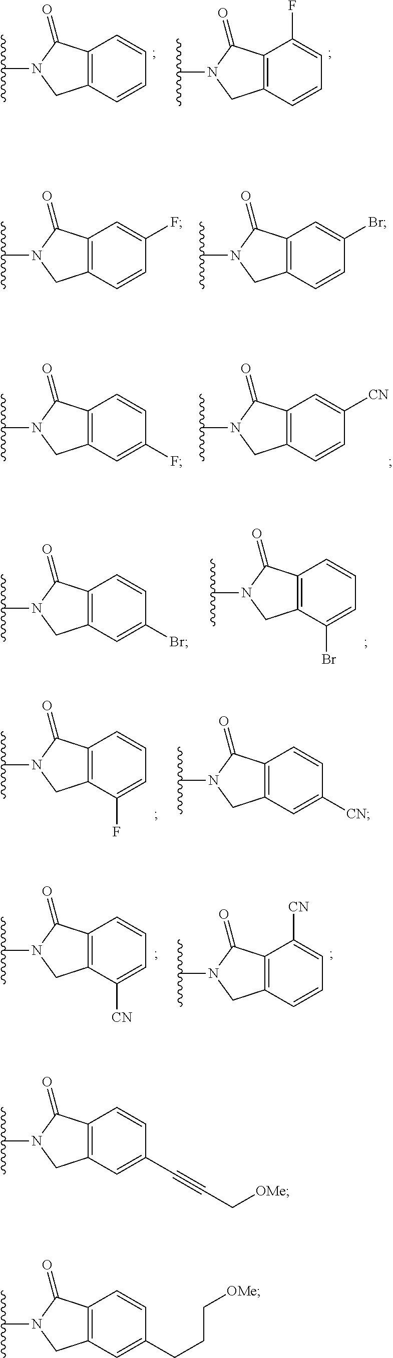

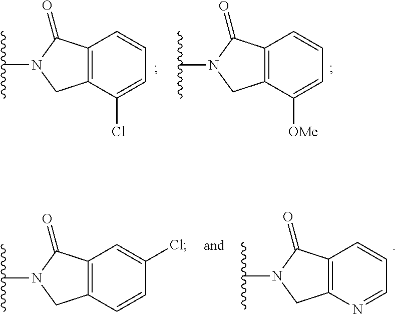

In certain embodiments, ULM has a chemical structure selected from the group of:

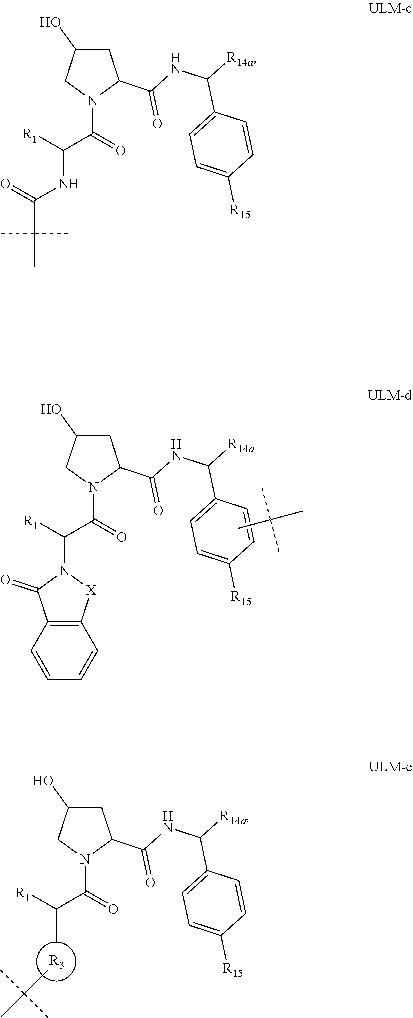

wherein:
R₁ of Formulas ULM-c, ULM-d, and ULM-e is H, ethyl, isopropyl, tert-butyl, sec-butyl, cyclopropyl, cyclobutyl, cyclopentyl, or cyclohexyl; optionally substituted alkyl, optionally substituted hydroxyalkyl, optionally substituted heteroaryl, or haloalkyl;
R₁₄ₐ of Formulas ULM-c, ULM-d, and ULM-e is H, haloalkyl, optionally substituted alkyl, methyl, fluoromethyl, hydroxymethyl, ethyl, isopropyl, or cyclopropyl;
R₁₅ of Formulas ULM-c, ULM-d, and ULM-e is selected from the group consisting of H, halogen, CN, OH, NO₂, optionally substituted heteroaryl, optionally substituted aryl;
optionally substituted alkyl, optionally substituted haloalkyl, optionally substituted haloalkoxy, optionally substituted cycloalkyl, or optionally substituted cycloheteroalkyl;
X of Formulas ULM-c, ULM-d, and ULM-e is C, CH₂, or C═O
R₃ of Formulas ULM-c, ULM-d, and ULM-e is absent or an optionally substituted 5 or 6 membered heteroaryl; and
the dashed line indicates the site of attachment of at least one PTM, another ULM (ULM′) or a chemical linker moiety coupling at least one PTM or a ULM′ or both to ULM.

In certain embodiments, ULM comprises a group according to the chemical structure:

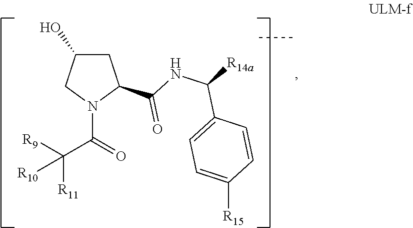

wherein:
R₁₄ₐ of Formula ULM-f is H, haloalkyl, optionally substituted alkyl, methyl, fluoromethyl, hydroxymethyl, ethyl, isopropyl, or cyclopropyl;
R₉ of Formula ULM-f is H;
R₁₀ of Formula ULM-f is H, ethyl, isopropyl, tert-butyl, sec-butyl, cyclopropyl, cyclobutyl, cyclopentyl, or cyclohexyl;
R₁₁ of Formula ULM-f is

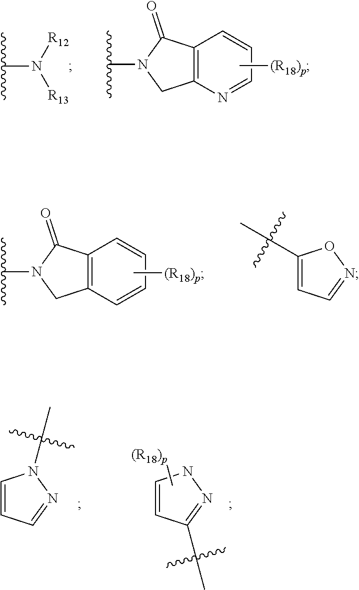

or optionally substituted heteroaryl;
p of Formula ULM-f is 0, 1, 2, 3, or 4;
each Rig of Formula ULM-f is independently halo, optionally substituted alkoxy, cyano, optionally substituted alkyl, haloalkyl, haloalkoxy or a linker;
R₁₂ of Formula ULM-f is H, C═O;
R₁₃ of Formula ULM-f is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclylcarbonyl, or optionally substituted aralkyl,
R₁₅ of Formula ULM-f is selected from the group consisting of H, halogen, Cl, CN, OH, NO₂, optionally substituted heteroaryl, optionally substituted aryl;

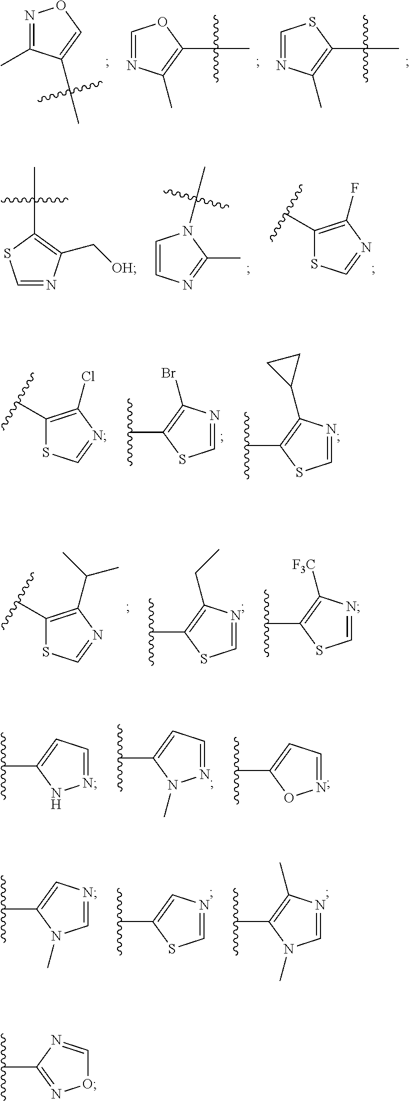

and
the dashed line of Formula ULM-f indicates the site of attachment of at least one PTM, another ULM (ULM′) or a chemical linker moiety coupling at least one PTM or a ULM′ or both to ULM.

In certain embodiments, the ULM is selected from the following structures:

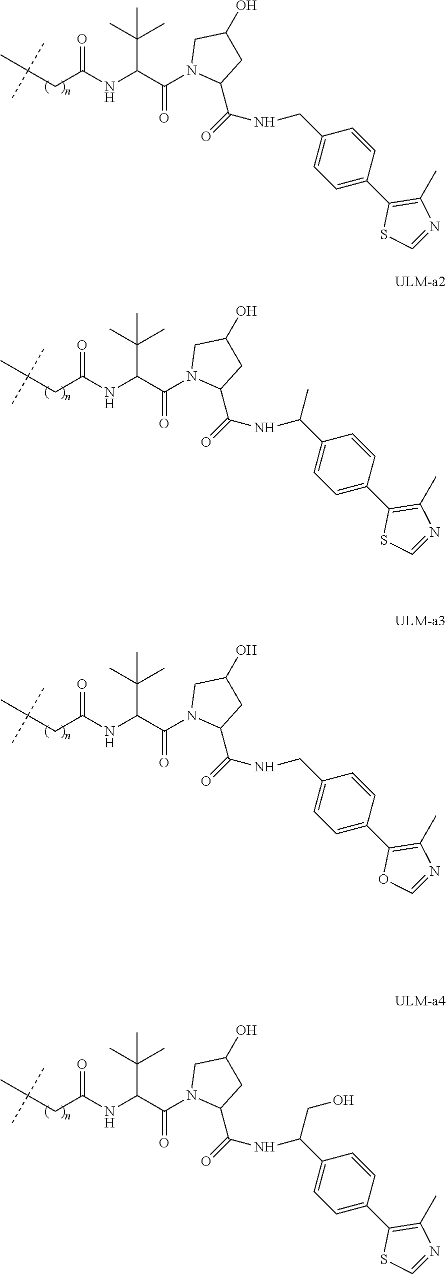

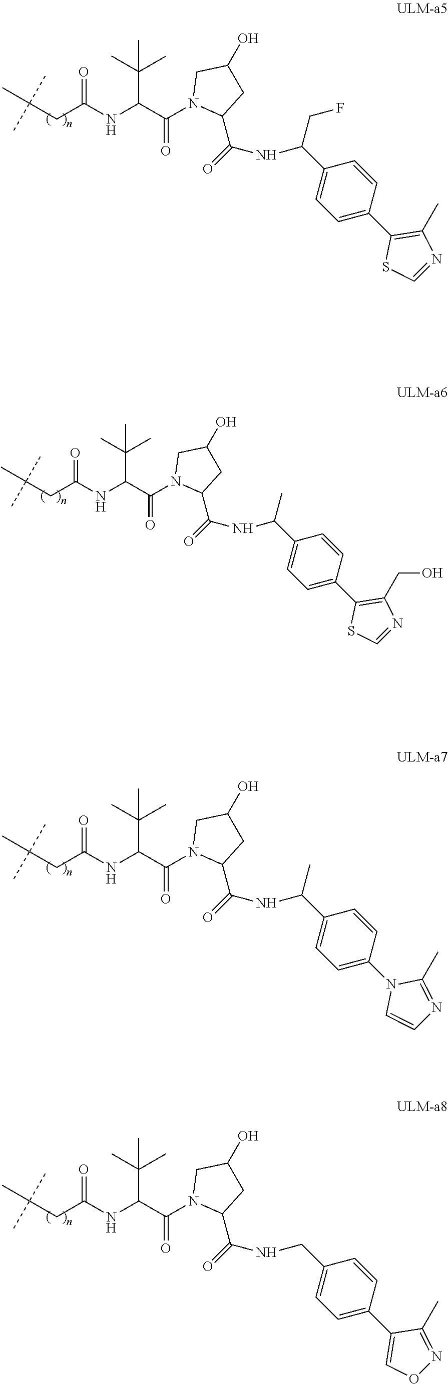

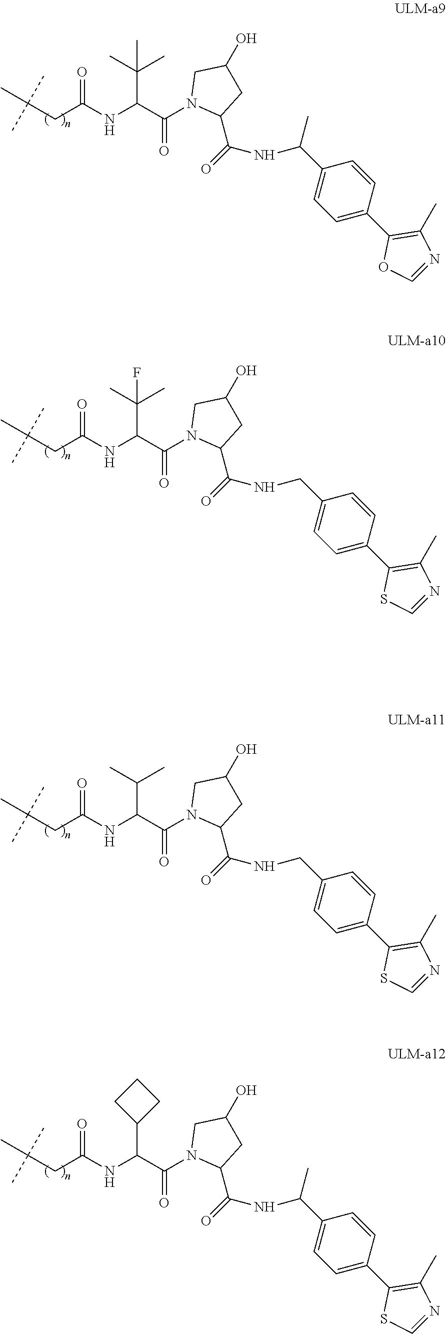

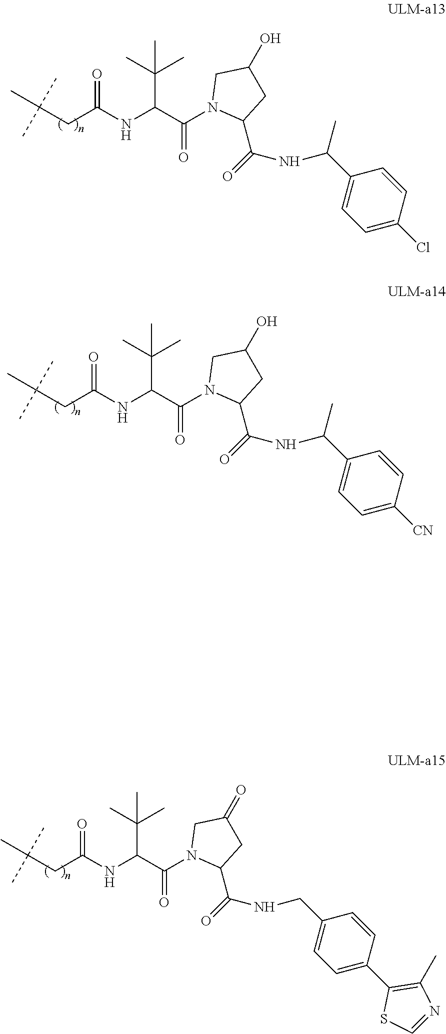

wherein n is 0 or 1.

In certain embodiments, the ULM is selected from the following structures:

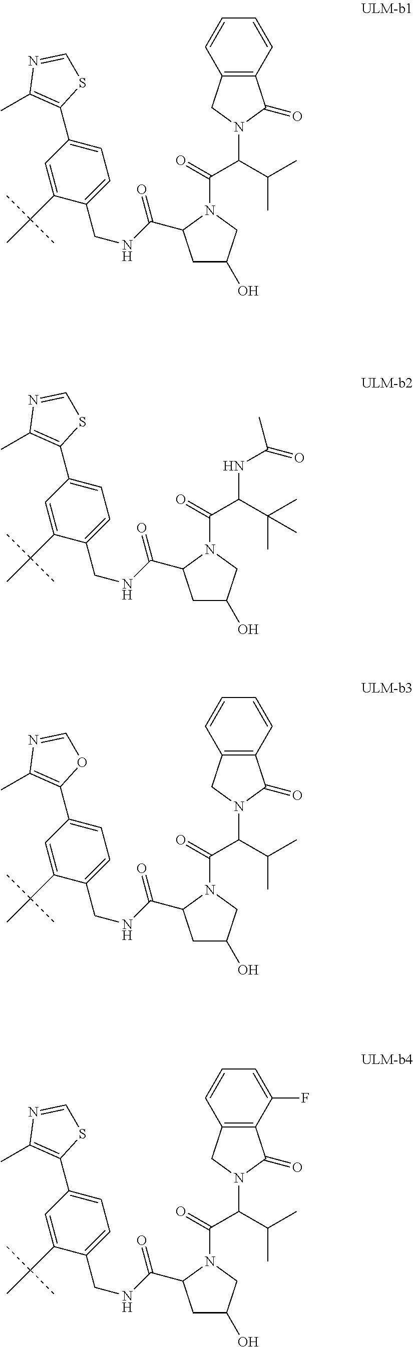

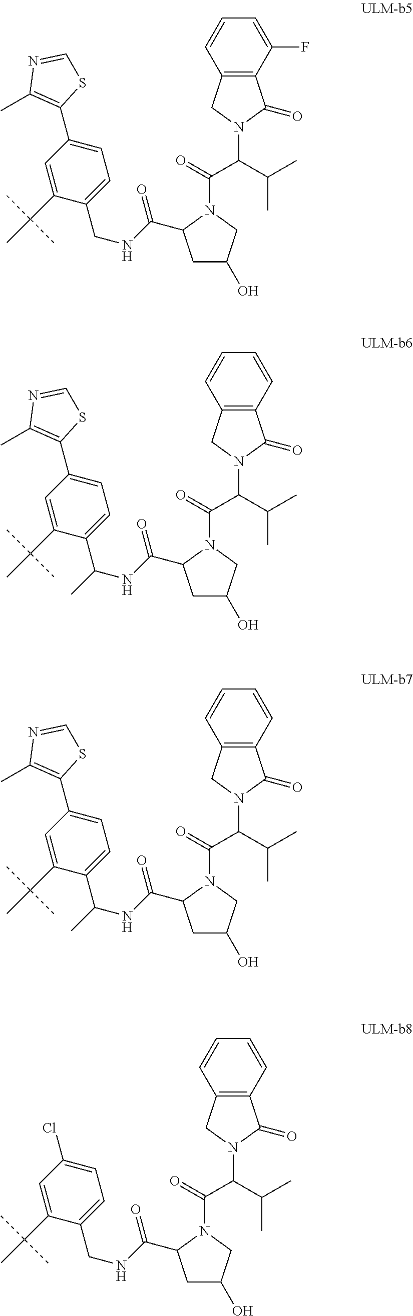

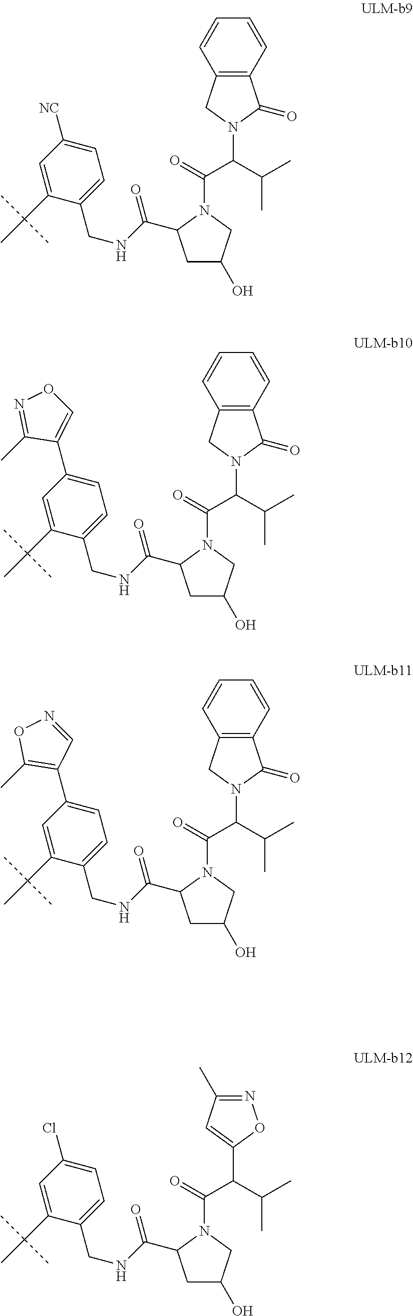

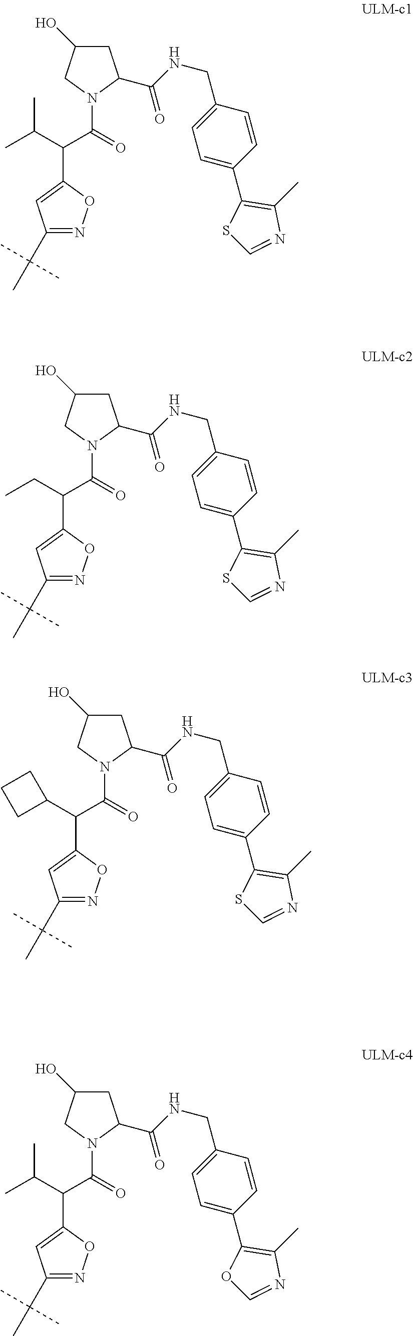

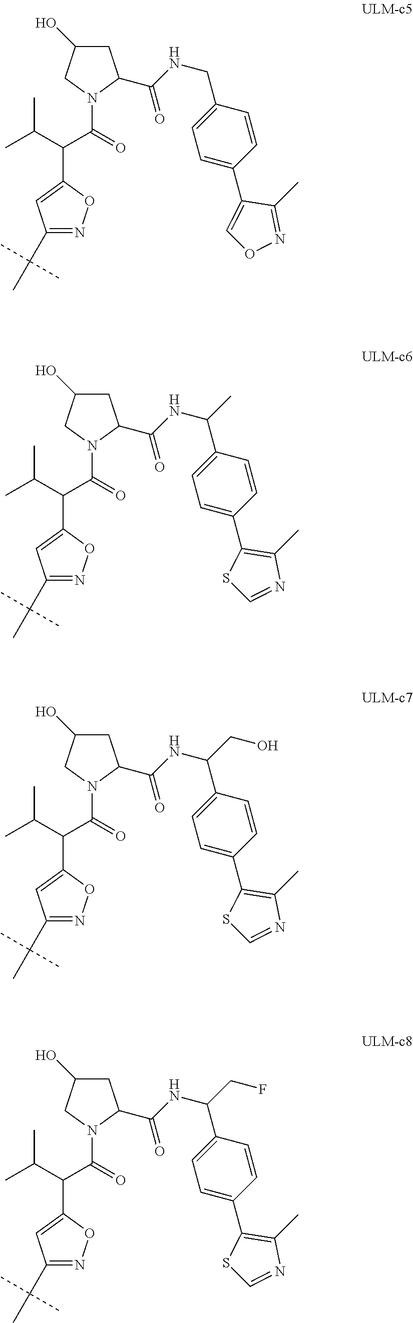

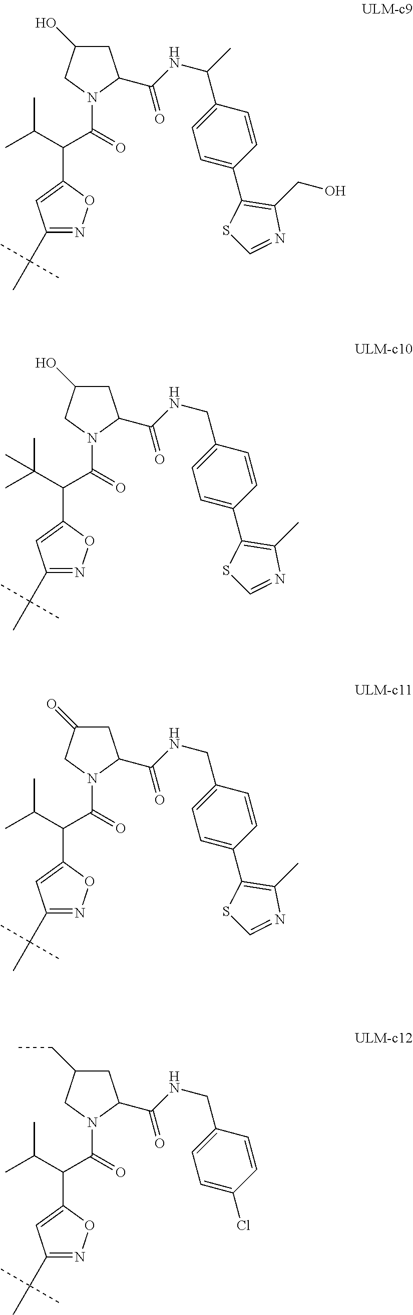

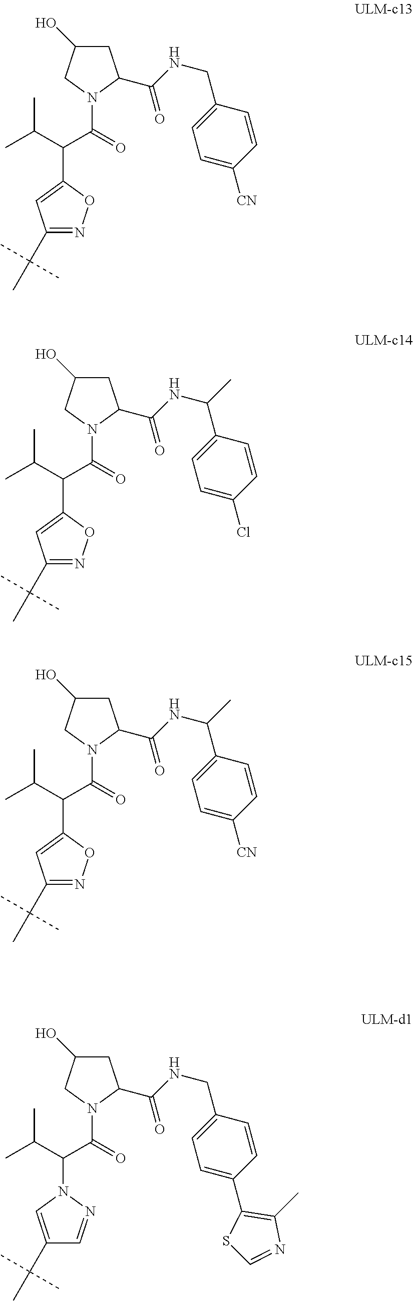

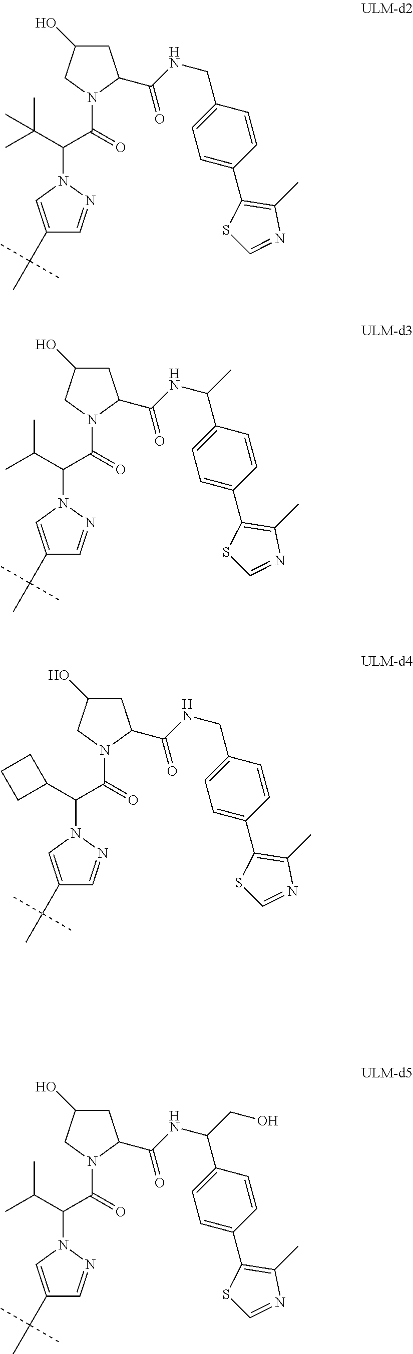

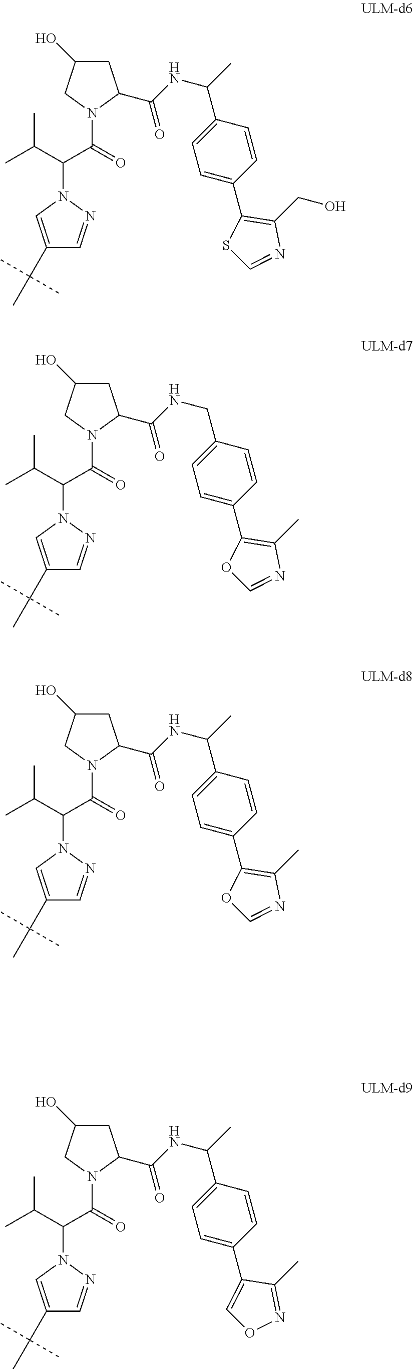

wherein, the phenyl ring in ULM-al through ULM -a15, ULM-b1 through ULM-b12, ULM-c1 through ULM-c15 and ULM-d1 through ULM-d9 is optionally substituted with fluorine, lower alkyl and alkoxy groups, and wherein the dashed line indicates the site of attachment of at least one PTM, another ULM (ULM′) or a chemical linker moiety coupling at least one PTM or a ULM′ or both to ULM-a.

In one embodiment, the phenyl ring in ULM-al through ULM-a15, ULM-b1 through ULM-b12, ULM-c1 through ULM-c15 and ULM-d1 through ULM-d9 can be functionalized as the ester to make it a part of the prodrug.

In certain embodiments, the hydroxyl group on the pyrrolidine ring of ULM-al through ULM-a15, ULM-b1 through ULM-b12, ULM-c1 through ULM-c15 and ULM-d1 through ULM-d9, respectively, comprises an ester-linked prodrug moiety.

In any of the aspects or embodiments described herein, the ULM and where present, ULM′, are each independently a group according to the chemical structure:

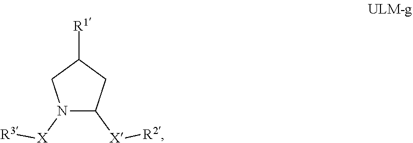

wherein:
R¹ of ULM-g is an optionally substituted C₁-C₆ alkyl group, an optionally substituted —(CH₂)ₙOH, an optionally substituted —(CH₂)ₙSH, an optionally substituted (CH₂)ₙ—O—(C₁-C₆)alkyl group, an optionally substituted (CH₂)ₙ—WCOCW—(C₀-C₆)alkyl group containing an epoxide moiety WCOCW where each W is independently H or a C₁-C₃ alkyl group, an optionally substituted —(CH₂)ₙCOOH, an optionally substituted —(CH₂)ₙC(O)—(C₁-C₆ alkyl), an optionally substituted —(CH₂)ₙNHC(O)—R₁, an optionally substituted —(CH₂)ₙC(O)—NR₁R₂, an optionally substituted —(CH₂)ₙOC(O)—NR₁R₂, —(CH₂O)ₙH, an optionally substituted —(CH₂)ₙOC(O)—(C₁-C₆ alkyl), an optionally substituted —(CH₂)ₙC(O)—O—(C₁-C₆ alkyl), an optionally substituted —(CH₂O)ₙCOOH, an optionally substituted —(OCH₂)ₙO—(C₁-C₆ alkyl), an optionally substituted —(CH₂O)ₙC(O)—(C₁-C₆ alkyl), an optionally substituted —(OCH₂)ₙNHC(O)—R₁, an optionally substituted —(CH₂O)ₙC(O)—NR₁R₂, —(CH₂CH₂O)ₙH, an optionally substituted —(CH₂CH₂O)ₙCOOH, an optionally substituted —(OCH₂CH₂)ₙO—(C₁-C₆ alkyl), an optionally substituted —(CH₂CH₂O)ₙC(O)—(C₁-C₆ alkyl), an optionally substituted —(OCH₂CH₂)ₙNHC(O)—R₁, an optionally substituted —(CH₂CH₂O)ₙC(O)—NR₁R₂, an optionally substituted —SO₂R₅, an optionally substituted S(O)R₅, NO₂, CN or halogen (F, Cl, Br, I, preferably F or Cl);
R₁ and R₂ of ULM-g are each independently H or a C₁-C₆ alkyl group which may be optionally substituted with one or two hydroxyl groups or up to three halogen groups (preferably fluorine);
R_{S} of ULM-g is a C₁-C₆alkyl group, an optionally substituted aryl, heteroaryl or heterocycle group or a —(CH₂)ₘNR₁R₂ group;
X and X′ of ULM-g are each independently C═O, C═S, —S(O), S(O)₂, (preferably X and X′ are both C═O);
R² of ULM-g is an optionally substituted —(CH₂)ₙ—(C═O)ᵤ(NR₁)ᵥ(SO₂)_{w}alkyl group, an optionally substituted —(CH₂)ₙ—(C═O)ᵤ(NR₁)ᵥ(SO₂)_{w}NR_{1N}R_{2N} group, an optionally substituted —(CH₂)ₙ—(C═O)ᵤ(NR₁)ᵥ(SO₂)_{w}-Aryl, an optionally substituted —(CH₂)ₙ—(C═O)ᵤ(NR₁)ᵥ(SO₂)_{w}-Heteroaryl, an optionally substituted —(CH₂)ₙ—(C═O)ᵥNR₁(SO₂)_{w}-Heterocycle, an optionally substituted —NR¹—(CH₂)ₙ—C(O)ᵤ(NR₁)ᵥ(SO₂)_{w}-alkyl, an optionally substituted —NR¹—(CH₂)ₙ—C(O)ᵤ(NR₁)ᵥ(SO₂)_{w}—NR_{1N}R_{2N}, an optionally substituted —NR¹—(CH₂)ₙ—C(O)ᵤ(NR₁)ᵥ(SO₂)_{w}—NR₁C(O)R_{1N}, an optionally substituted —NR¹—(CH₂)ₙ—(C═O)ᵤ(NR₁)ᵥ(SO₂)_{w}-Aryl, an optionally substituted —NR¹—(CH₂)ₙ—(C═O)ᵤ(NR₁)ᵥ(SO₂)_{w}-Heteroaryl or an optionally substituted —NR¹—(CH₂)ₙ—(C═O)ᵥNR₁(SO₂)_{w}—Heterocycle, an optionally substituted —X^{R2′}-alkyl group; an optionally substituted —X^{R2′}-Aryl group; an optionally substituted —X^{R2′}-Heteroaryl group; an optionally substituted —X^{R2′}-Heterocycle group; an optionally substituted;
R^{3′} of ULM-g is an optionally substituted alkyl, an optionally substituted —(CH₂)ₙ—(O)ᵤ(NR₁)ᵥ(SO₂)_{w}-alkyl, an optionally substituted —(CH₂)ₙ—C(O)ᵤ(NR₁)ᵥ(SO₂)_{w}—NR_{1N}R_{2N}, an optionally substituted —(CH₂)ₙ—C(O)ᵤ(NR₁)ᵥ(SO₂)_{w}—NR₁C(O)R_{1N}, an optionally substituted —(CH₂)ₙ—C(O)ᵤ(NR₁)ᵥ(SO₂)_{w}—C(O)NR₁R₂, an optionally substituted —(CH₂)ₙ—C(O)ᵤ(NR₁)ᵥ(SO₂)_{w}-Aryl, an optionally substituted —(CH₂)ₙ—C(O)ᵤ(NR₁)ᵥ(SO₂)_{w}-Heteroaryl, an optionally substituted —(CH₂)ₙ—C(O)ᵤ(NR₁)ᵥ(SO₂)_{w}-Heterocycle, an optionally substituted —NR¹—(CH₂)ₙ—C(O)ᵤ(NR₁)ᵥ(SO₂)_{w}-alkyl, an optionally substituted —NR¹—(CH₂)ₙ—C(O)ᵤ(NR₁)ᵥ(SO₂)_{w}—NR_{1N}R_{2N}, an optionally substituted —NR¹—(CH₂)ₙ—C(O)ᵤ(NR₁)ᵥ(SO₂)_{w}—NR₁C(O)R_{1N}, an optionally substituted —NR¹—(CH₂)ₙ—C(O)ᵤ(NR₁)ᵥ(SO2)_{w}-Aryl, an optionally substituted —NR¹—(CH₂)ₙ—C(O)ᵤ(NR₁)ᵥ(SO₂)_{w}-Heteroaryl, an optionally substituted —NR¹—(CH₂)ₙ—C(O)ᵤ(NR₁)ᵥ(SO₂)_{w}-Heterocycle, an optionally substituted —O—(CH₂)n-(C═O)ᵤ(NR₁)ᵥ(SO₂)_{w}-alkyl, an optionally substituted —O—(CH₂)ₙ—(C═O)ᵤ(NR₁)ᵥ(SO₂)_{w}—NR_{1N}R_{2N}, an optionally substituted —O—(CH₂)n-(C═O)ᵤ(NR₁)ᵥ(SO₂)_{w}—NR₁C(O)R_{1N}, an optionally substituted —O—(CH₂)n-(C═O)ᵤ(NR₁)ᵥ(SO₂)_{w}-Aryl, an optionally substituted —O—(CH₂)ₙ—(C═O)ᵤ(NR₁)ᵥ(SO₂)_{w}-Heteroaryl or an optionally substituted —O—(CH₂)ₙ—(C═O)ᵤ(NR₁)ᵥ(SO₂)_{w}-Heterocycle; —(CH₂)ₙ—(V)_{n′}—(CH₂)ₙ—(V)_{n′}-alkyl group, an optionally substituted —(CH₂)ₙ—(V)_{n′}—(CH₂)ₙ—(V)_{n′}-Aryl group, an optionally substituted —(CH₂)ₙ—(V)_{n′}—(CH₂)ₙ—(V)_{n′}-Heteroaryl group, an optionally substituted —(CH₂)ₙ—(V)_{n′}—(CH₂)ₙ—(V)_{n′}-Heterocycle group, an optionally substituted —(CH₂)ₙ—N(R_{1′})(C═O)_{m′}—(V)_{n′}-alkyl group, an optionally substituted —(CH₂)ₙ—N(R_{1′})(C═O)_{m′}—(V)_{n′}-Aryl group, an optionally substituted —(CH₂)ₙ—N(R_{1′})(C═O)_{m′}—(V)_{n′}-Heteroaryl group, an optionally substituted —(CH₂)ₙ—N(R_{1′})(C═O)_{m′}—(V)_{n′}-Heterocycle group, an optionally substituted —X^{R3′}-alkyl group; an optionally substituted —X^{R3′}-Aryl group; an optionally substituted —X^{R3′}-Heteroaryl group; an optionally substituted —X^{R3′}-Heterocycle group; an optionally substituted;
R_{1N} and R_{2N} of ULM-g are each independently H, C₁-C₆ alkyl which is optionally substituted with one or two hydroxyl groups and up to three halogen groups or an optionally substituted —(CH₂)ₙ-Aryl, —(CH₂)ₙ-Heteroaryl or —(CH₂)ₙ-Heterocycle group;
V of ULM-g is O, S or NR₁;
R₁ of ULM-g is the same as above;
R¹ and R_{1′} of ULM-g are each independently H or a C₁-C₃ alkyl group;
X^{R2′} and X^{R3′} of ULM-g are each independently an optionally substituted —CH₂)ₙ—, —CH₂)ₙ—CH(Xᵥ)═CH(Xᵥ)-(cis or trans), —CH₂)ₙ—CH═CH—, —(CH₂CH₂O)ₙ— or a C₃-C₆ cycloalkyl group, where Xᵥ is H, a halo or a C₁-C₃ alkyl group which is optionally substituted;
each m of ULM-g is independently 0, 1, 2, 3, 4, 5, 6;
each m′ of ULM-g is independently 0 or 1;
each n of ULM-g is independently 0, 1, 2, 3, 4, 5, 6;
each n′ of ULM-g is independently 0 or 1;
each u of ULM-g is independently 0 or 1;
each v of ULM-g is independently 0 or 1;
each w of ULM-g is independently 0 or 1; and
any one or more of R¹, R², R³, X and X′ of ULM-g is optionally modified to be covalently bonded to the PTM group through a linker group when PTM is not ULM′, or when PTM is ULM′, any one or more of R¹, R², R³, X and X′ of each of ULM and ULM′ are optionally modified to be covalently bonded to each other directly or through a linker group, or a pharmaceutically acceptable salt, stereoisomer, solvate or polymorph thereof.

In any of the aspects or embodiments described herein, the ULM and when present, ULM′, are each independently a group according to the chemical structure:

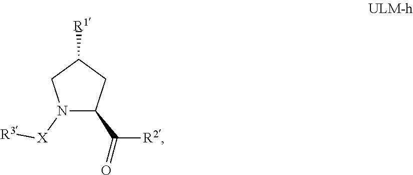

wherein:
each of R^{1′}, R^{2′} and R^{3′} of ULM-h are the same as above and X is C═O, C═S, —S(O) group or a S(O)₂ group, more preferably a C═O group, and
any one or more of R^{1′}, R^{2′} and R^{3′} of ULM-h are optionally modified to bind a linker group to which is further covalently bonded to the PTM group when PTM is not ULM′, or when PTM is ULM′, any one or more of R^{1′}, R^{2′}, R^{3′} of each of ULM and ULM′ are optionally modified to be covalently bonded to each other directly or through a linker group, or
a pharmaceutically acceptable salt, enantiomer, diastereomer, solvate or polymorph thereof.

In any of the aspects or embodiments described herein, the ULM, and when present, ULM′, are each independently according to the chemical structure:

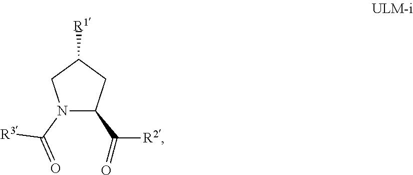

wherein:
any one or more of R^{1′}, R^{2′} and R^{3′} of ULM-I are optionally modified to bind a linker group to which is further covalently bonded to the PTM group when PTM is not ULM′, or when PTM is ULM′, any one or more of R^{1′}, R^{2′}, R^{3′} of each of ULM and ULM′ are optionally modified to be covalently bonded to each other directly or through a linker group, or
a pharmaceutically acceptable salt, enantiomer, diastereomer, solvate or polymorph thereof.

In further preferred aspects of the disclosure, R^{1′} of ULM-g through ULM-i is preferably a hydroxyl group or a group which may be metabolized to a hydroxyl or carboxylic group, such that the compound represents a prodrug form of an active compound. Exemplary preferred R^{1′} groups include, for example, —(CH₂)ₙOH, (CH₂)ₙ—O—(C₁-C₆)alkyl group, —(CH₂)ₙCOOH, (CH₂O)ₙH, an optionally substituted —(CH₂)ₙOC(O)—(C₁-C₆ alkyl), or an optionally substituted —(CH₂)ₙC(O)—O—(C₁-C₆ alkyl), wherein n is 0 or 1. Where R^{1′} is or contains a carboxylic acid group, a hydroxyl group or an amine group, the hydroxyl group, carboxylic acid group or amine (each of which may be optionally substituted), may be further chemically modified to provide a covalent link to a linker group to which the PTM group (including a ULM′ group) is bonded;

X and X′, where present, of ULM-g and ULM-h are preferably a C═O, C═S, —S(O) group or a S(O)₂ group, more preferably a C═O group;

R^{2′} of ULM-g through ULM-i is preferably an optionally substituted —NR¹-T-Aryl, an optionally substituted —NR¹-T-Heteroaryl group or an optionally substituted —NR¹-T-Heterocycle, where R¹ is H or CH₃, preferably H and T is an optionally substituted —(CH₂)ₙ— group, wherein each one of the methylene groups may be optionally substituted with one or two substituents, preferably selected from halogen, an amino acid sidechain as otherwise described herein or a C₁-C₃ alkyl group, preferably one or two methyl groups, which may be optionally substituted; and n is 0 to 6, often 0, 1, 2 or 3, preferably 0 or 1. Alternatively, T may also be a —(CH₂O)ₙ— group, a —(OCH₂)ₙ— group, a —(CH₂CH₂O)ₙ— group, a —(OCH₂CH₂)ₙ— group, all of which groups are optionally substituted.

Preferred Aryl groups for R² of ULM-g through ULM-i include optionally substituted phenyl or naphthyl groups, preferably phenyl groups, wherein the phenyl or naphthyl group is connected to a PTM (including a ULM′ group) with a linker group and/or optionally substituted with a halogen (preferably F or Cl), an amine, monoalkyl- or dialkyl amine (preferably, dimethylamine), F, Cl, OH, COOH, C₁-C₆ alkyl, preferably CH₃, CF₃, OMe, OCF₃, NO₂, or CN group (each of which may be substituted in ortho-, meta- and/or para-positions of the phenyl ring, preferably para-), an optionally substituted phenyl group (the phenyl group itself is optionally connected to a PTM group, including a ULM′, with a linker group), and/or optionally substituted with at least one of F, Cl, OH, COOH, CH₃, CF₃, OMe, OCF₃, NO₂, or CN group (in ortho-, meta- and/or para-positions of the phenyl ring, preferably para-), a naphthyl group, which may be optionally substituted, an optionally substituted heteroaryl, preferably an optionally substituted isoxazole including a methylsubstituted isoxazole, an optionally substituted oxazole including a methylsubstituted oxazole, an optionally substituted thiazole including a methyl substituted thiazole, an optionally substituted isothiazole including a methyl substituted isothiazole, an optionally substituted pyrrole including a methylsubstituted pyrrole, an optionally substituted imidazole including a methylimidazole, an optionally substituted benzimidazole or methoxybenzylimidazole, an optionally substituted oximidazole or methyloximidazole, an optionally substituted diazole group, including a methyldiazole group, an optionally substituted triazole group, including a methylsubstituted triazole group, an optionally substituted pyridine group, including a halo-(preferably, F) or methylsubstitutedpyridine group or an oxapyridine group (where the pyridine group is linked to the phenyl group by an oxygen), an optionally substituted furan, an optionally substituted benzofuran, an optionally substituted dihydrobenzofuran, an optionally substituted indole, indolizine or azaindolizine (2, 3, or 4-azaindolizine), an optionally substituted quinoline, an optionally substituted group according to the chemical structure:

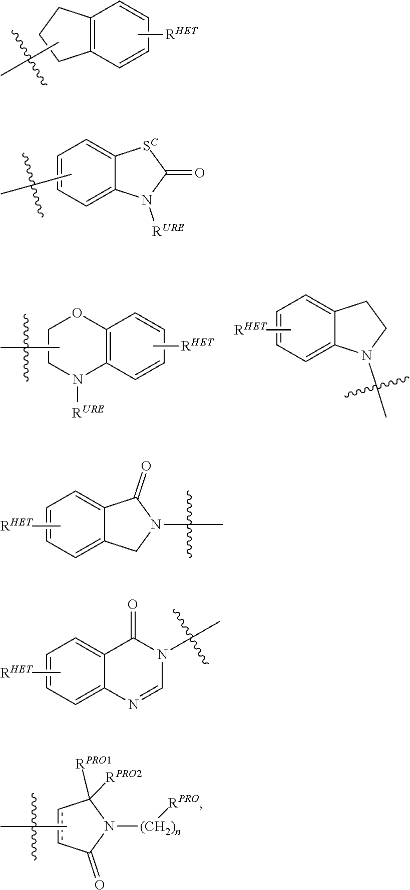

wherein:
S^{c} of ULM-g through ULM-i is CHR^{SS}, NR^{URE}, or O;
R^{HET} of ULM-g through ULM-i is H, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl);
R^{SS} of ULM-g through ULM-i is H, CN, NO₂, halo (preferably F or Cl), optionally substituted C₁-C₆ alkyl (preferably substituted with one or two hydroxyl groups or up to three halo groups), optionally substituted O—(C₁-C₆ alkyl) (preferably substituted with one or two hydroxyl groups or up to three halo groups) or an optionally substituted —C(O)(C₁-C₆ alkyl) (preferably substituted with one or two hydroxyl groups or up to three halo groups);
R^{URE} of ULM-g through ULM-i is H, a C₁-C₆ alkyl (preferably H or C₁-C₃ alkyl) or a —C(O)(C₁-C₆ alkyl) each of which groups is optionally substituted with one or two hydroxyl groups or up to three halogen, preferably fluorine groups, or an optionally substituted phenyl group, an optionally substituted heteroaryl, or an optionally substituted heterocycle, preferably for example piperidine, morpholine, pyrrolidine, tetrahydrofuran);
R^{PRO} of ULM-g through ULM-i is H, optionally substituted C₁-C₆ alkyl or an optionally substituted aryl (phenyl or napthyl), heteroaryl or heterocyclic group selected from the group consisting of oxazole, isoxazole, thiazole, isothiazole, imidazole, diazole, oximidazole, pyrrole, pyrollidine, furan, dihydrofuran, tetrahydrofuran, thiene, dihydrothiene, tetrahydrothiene, pyridine, piperidine, piperazine, morpholine, quinoline, (each preferably substituted with a C₁-C₃ alkyl group, preferably methyl or a halo group, preferably F or Cl), benzofuran, indole, indolizine, azaindolizine;
R^{PRO1} and R^{PRO2 of ULM-g through ULM-i} are each independently H, an optionally substituted C₁-C₃ alkyl group or together form a keto group; and
each n of ULM-g through ULM-i is independently 0, 1, 2, 3, 4, 5, or 6 (preferably 0 or 1), or an optionally substituted heterocycle, preferably tetrahydrofuran, tetrahydrothiene, piperidine, piperazine or morpholine (each of which groups when substituted, are preferably substituted with a methyl or halo (F, Br, Cl), each of which groups may be optionally attached to a PTM group (including a ULM′ group) via a linker group.

In certain preferred aspects,

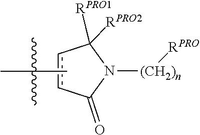

of ULM-g through ULM-i is a

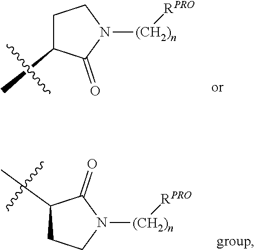

where R^{PRO} and n of ULM-g through ULM-i are the same as above.

Preferred heteroaryl groups for R² of ULM-g through ULM-i include an optionally substituted quinoline (which may be attached to the pharmacophore or substituted on any carbon atom within the quinoline ring), an optionally substituted indole, an optionally substituted indolizine, an optionally substituted azaindolizine, an optionally substituted benzofuran, including an optionally substituted benzofuran, an optionally substituted isoxazole, an optionally substituted thiazole, an optionally substituted isothiazole, an optionally substituted thiophene, an optionally substituted pyridine (2-, 3, or 4-pyridine), an optionally substituted imidazole, an optionally substituted pyrrole, an optionally substituted diazole, an optionally substituted triazole, a tetrazole, an optionally substituted oximidazole, or a group according to the chemical structure:

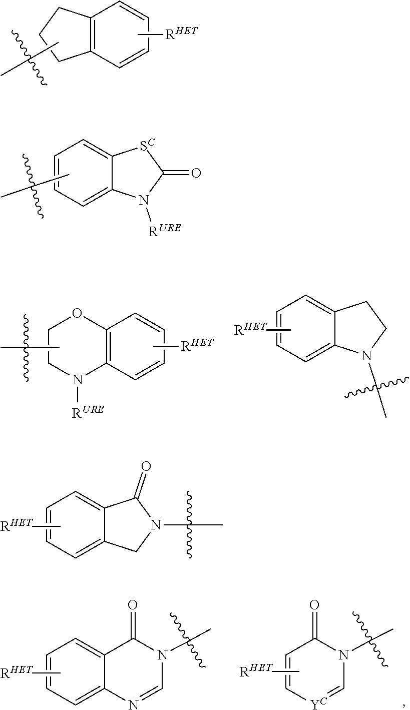

wherein:
S^{c} of ULM-g through ULM-i is CHR^{SS}, NR^{URE}, or O;
R^{HET} ULM-g through ULM-i is H, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ of ULM-g through ULM-i is H or a C₁-C₆ alkyl group (preferably C₁-C₃ alkyl);
R^{SS} of ULM-g through ULM-i is H, CN, NO₂, halo (preferably F or Cl), optionally substituted C₁-C₆ alkyl (preferably substituted with one or two hydroxyl groups or up to three halo groups), optionally substituted O—(C₁-C₆ alkyl) (preferably substituted with one or two hydroxyl groups or up to three halo groups) or an optionally substituted —C(O)(C₁-C₆ alkyl) (preferably substituted with one or two hydroxyl groups or up to three halo groups);
R^{URE} of ULM-g through ULM-i is H, a C₁-C₆ alkyl (preferably H or C₁-C₃ alkyl) or a —C(O)(C₁-C₆ alkyl), each of which groups is optionally substituted with one or two hydroxyl groups or up to three halogen, preferably fluorine groups, or an optionally substituted heterocycle, for example piperidine, morpholine, pyrrolidine, tetrahydrofuran, tetrahydrothiophene, piperidine, piperazine, each of which is optionally substituted, and
Y^{C} of ULM-g through ULM-i is N or C—R^{YC}, where R^{YC} is H, OH, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl), each of which groups may be optionally connected to a PTM group (including a ULM′ group) via a linker group.

Preferred heterocycle groups for R^{2′} of ULM-g through ULM-i include tetrahydrofuran, tetrahydrothiene, tetrahydroquinoline, piperidine, piperazine, pyrrollidine, morpholine, oxane or thiane, each of which groups may be optionally substituted, or a group according to the chemical structure:

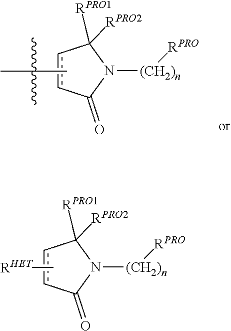

preferably, a

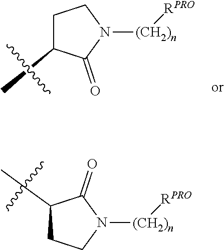

group,
wherein:
R^{PRO} ULM-g through ULM-i is H, optionally substituted C₁-C₆ alkyl or an optionally substituted aryl, heteroaryl or heterocyclic group;
R^{PRO1} and R^{PRO2} of ULM-g through ULM-i are each independently H, an optionally substituted C₁-C₃ alkyl group or together form a keto group and
each n of ULM-g through ULM-i is independently 0, 1, 2, 3, 4, 5, or 6 (often 0 or 1), each of which groups may be optionally connected to a PTM group (including a ULM′ group) via a linker group.

Preferred R^{2′} substituents of ULM-g through ULM-i also include specifically (and without limitation to the specific compound disclosed) the R^{2′} substituents which are found in the identified compounds disclosed herein (which includes the specific compounds which are disclosed in the present specification, and the figures which are attached hereto). Each of these R^{2′} substituents may be used in conjunction with any number of R^{3′} substituents which are also disclosed herein.

R^{3′} of ULM-g through ULM-i is preferably an optionally substituted -T-Aryl, an optionally substituted-T-Heteroaryl, an optionally substituted -T-Heterocycle, an optionally substituted-NR¹-T-Aryl, an optionally substituted —NR¹-T-Heteroaryl or an optionally substituted-NR¹-T-Heterocycle, where R¹ is H or a C₁-C₃ alkyl group, preferably H or CH₃, T is an optionally substituted —(CH₂)ₙ— group, wherein each one of the methylene groups may be optionally substituted with one or two substituents, preferably selected from halogen, a C₁-C₃ alkyl group or the sidechain of an amino acid as otherwise described herein, preferably methyl, which may be optionally substituted; and n is 0 to 6, often 0, 1, 2, or 3 preferably 0 or 1. Alternatively, T may also be a —(CH₂O)ₙ— group, a —(OCH₂)ₙ— group, a —(CH₂CH₂O)ₙ— group, a —(OCH₂CH₂)ₙ— group, each of which groups is optionally substituted.

Preferred aryl groups for R^{3′} of ULM-g through ULM-i include optionally substituted phenyl or naphthyl groups, preferably phenyl groups, wherein the phenyl or naphthyl group is optionally connected to a PTM group (including a ULM′ group) via a linker group and/or optionally substituted with a halogen (preferably F or Cl), an amine, monoalkyl- or dialkyl amine (preferably, dimethylamine), an amido group (preferably a —(CH₂)ₘ—NR₁C(O)R₂ group where m, R₁ and R₂ are the same as above), a halo (often F or Cl), OH, CH₃, CF₃, OMe, OCF₃, NO₂, CN or a S(O)₂R₅ group (R₅ is a a C₁-C₆ alkyl group, an optionally substituted aryl, heteroaryl or heterocycle group or a —(CH₂)ₘNR₁R₂ group), each of which may be substituted in ortho-, meta- and/or para-positions of the phenyl ring, preferably para-), or an Aryl (preferably phenyl), Heteroaryl or Heterocycle. Preferably said substituent phenyl group is an optionally substituted phenyl group (i.e., the substituent phenyl group itself is preferably substituted with at least one of F, Cl, OH, SH, COOH, CH₃, CF₃, OMe, OCF₃, NO₂, CN or a linker group to which is attached a PTM group (including a ULM′ group), wherein the substitution occurs in ortho-, meta- and/or para-positions of the phenyl ring, preferably para-), a naphthyl group, which may be optionally substituted including as described above, an optionally substituted heteroaryl (preferably an optionally substituted isoxazole including a methylsubstituted isoxazole, an optionally substituted oxazole including a methylsubstituted oxazole, an optionally substituted thiazole including a methyl substituted thiazole, an optionally substituted pyrrole including a methylsubstituted pyrrole, an optionally substituted imidazole including a methylimidazole, a benzylimidazole or methoxybenzylimidazole, an oximidazole or methyloximidazole, an optionally substituted diazole group, including a methyldiazole group, an optionally substituted triazole group, including a methylsubstituted triazole group, a pyridine group, including a halo-(preferably, F) or methylsubstitutedpyridine group or an oxapyridine group (where the pyridine group is linked to the phenyl group by an oxygen) or an optionally substituted heterocycle (tetrahydrofuran, tetrahydrothiophene, pyrrolidine, piperidine, morpholine, piperazine, tetrahydroquinoline, oxane or thiane. Each of the aryl, heteroaryl or heterocyclic groups may be optionally connected to a PTM group (including a ULM′ group) via a linker group.

Preferred Heteroaryl groups for R³ of ULM-g through ULM-i include an optionally substituted quinoline (which may be attached to the pharmacophore or substituted on any carbon atom within the quinoline ring), an optionally substituted indole (including dihydroindole), an optionally substituted indolizine, an optionally substituted azaindolizine (2, 3 or 4-azaindolizine) an optionally substituted benzimidazole, benzodiazole, benzoxofuran, an optionally substituted imidazole, an optionally substituted isoxazole, an optionally substituted oxazole (preferably methyl substituted), an optionally substituted diazole, an optionally substituted triazole, a tetrazole, an optionally substituted benzofuran, an optionally substituted thiophene, an optionally substituted thiazole (preferably methyl and/or thiol substituted), an optionally substituted isothiazole, an optionally substituted triazole (preferably a 1,2,3-triazole substituted with a methyl group, a triisopropylsilyl group, an optionally substituted —(CH₂)ₘ—O—C₁-C₆ alkyl group or an optionally substituted —(CH₂)ₘ—C(O)—O—C₁-C₆ alkyl group), an optionally substituted pyridine (2-, 3, or 4-pyridine) or a group according to the chemical structure:

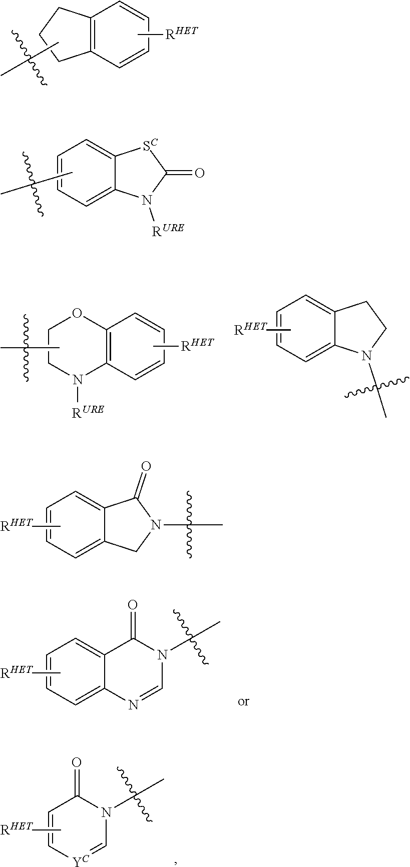

wherein:
S^{c} of ULM-g through ULM-i is CHR^{SS}, NR^{URE}, or O;
R^{HET} of ULM-g through ULM-i is H, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl);
R^{SS} of ULM-g through ULM-i is H, CN, NO₂, halo (preferably F or Cl), optionally substituted C₁-C₆ alkyl (preferably substituted with one or two hydroxyl groups or up to three halo groups), optionally substituted O—(C₁-C₆ alkyl) (preferably substituted with one or two hydroxyl groups or up to three halo groups) or an optionally substituted —C(O)(C₁-C₆ alkyl) (preferably substituted with one or two hydroxyl groups or up to three halo groups);
R^{URE} of ULM-g through ULM-i is H, a C₁-C₆ alkyl (preferably H or C₁-C₃ alkyl) or a —C(O)(C₁-C₆ alkyl), each of which groups is optionally substituted with one or two hydroxyl groups or up to three halogen, preferably fluorine groups, or an optionally substituted heterocycle, for example piperidine, morpholine, pyrrolidine, tetrahydrofuran, tetrahydrothiophene, piperidine, piperazine, each of which is optionally substituted, and
Y^{C} of ULM-g through ULM-i is N or C—R^{YC}, where R^{YC} is H, OH, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl). Each of said heteroaryl groups may be optionally connected to a PTM group (including a ULM′ group) via a linker group.

Preferred heterocycle groups for R³ of ULM-g through ULM-i include tetrahydroquinoline, piperidine, piperazine, pyrrollidine, morpholine, tetrahydrofuran, tetrahydrothiophene, oxane and thiane, each of which groups may be optionally substituted or a group according to the chemical structure:

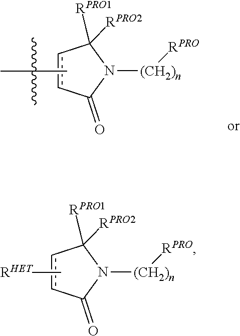

preferably, a

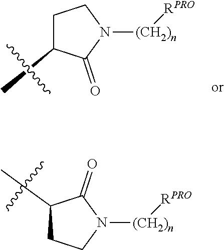

group,
wherein:
R^{PRO} ULM-g through ULM-i is H, optionally substituted C₁-C₆ alkyl or an optionally substituted aryl (phenyl or napthyl), heteroaryl or heterocyclic group selected from the group consisting of oxazole, isoxazole, thiazole, isothiazole, imidazole, diazole, oximidazole, pyrrole, pyrollidine, furan, dihydrofuran, tetrahydrofuran, thiene, dihydrothiene, tetrahydrothiene, pyridine, piperidine, piperazine, morpholine, quinoline, (each preferably substituted with a C₁-C₃ alkyl group, preferably methyl or a halo group, preferably F or Cl), benzofuran, indole, indolizine, azaindolizine;
R^{PRO1} and R^{PRO2} of ULM-g through ULM-i are each independently H, an optionally substituted C₁-C₃ alkyl group or together form a keto group, and each n of ULM-g through ULM-i is 0, 1, 2, 3, 4, 5, or 6 (preferably 0 or 1), wherein each of said Heteocycle groups may be optionally connected to a PTM group (including a ULM′ group) via a linker group.

Preferred R^{3′} substituents of ULM-g through ULM-i also include specifically (and without limitation to the specific compound disclosed) the R^{3′} substituents which are found in the identified compounds disclosed herein (which includes the specific compounds which are disclosed in the present specification, and the figures which are attached hereto). Each of these R^{3′} substituents may be used in conjunction with any number of R^{2′} substituents, which are also disclosed herein.

In certain alternative preferred embodiments, R^{2′} of ULM-g through ULM-i is an optionally substituted —NR₁—X^{R2′}-alkyl group, —NR₁—X^{R2′}-Aryl group; an optionally substituted —NR₁—X^{R2′}-HET, an optionally substituted —NR₁—X^{R2′}-Aryl-HET or an optionally substituted —NR₁—X^{R2′}-HET-Aryl,

wherein:
R₁ of ULM-g through ULM-i is H or a C₁-C₃ alkyl group (preferably H);
X^{R2′} of ULM-g through ULM-i is an optionally substituted —CH₂)ₙ—, —CH₂)ₙ—CH(Xᵥ)═CH(Xᵥ)-(cis or trans), —(CH₂)ₙ—CH═CH—, —(CH₂CH₂O)ₙ— or a C₃-C₆ cycloalkyl group; and
Xᵥ of ULM-g through ULM-i is H, a halo or a C₁-C₃ alkyl group which is optionally substituted with one or two hydroxyl groups or up to three halogen groups;
Alkyl of ULM-g through ULM-i is an optionally substituted C1-C₁₀ alkyl (preferably a C₁-C₆ alkyl) group (in certain preferred embodiments, the alkyl group is end-capped with a halo group, often a Cl or Br);
Aryl of ULM-g through ULM-i is an optionally substituted phenyl or naphthyl group (preferably, a phenyl group); and
HET of ULM-g through ULM-i is an optionally substituted oxazole, isoxazole, thiazole, isothiazole, imidazole, diazole, oximidazole, pyrrole, pyrollidine, furan, dihydrofuran, tetrahydrofuran, thiene, dihydrothiene, tetrahydrothiene, pyridine, piperidine, piperazine, morpholine, benzofuran, indole, indolizine, azaindolizine, quinoline (when substituted, each preferably substituted with a C₁-C₃ alkyl group, preferably methyl or a halo group, preferably F or Cl) or a group according to the chemical structure:

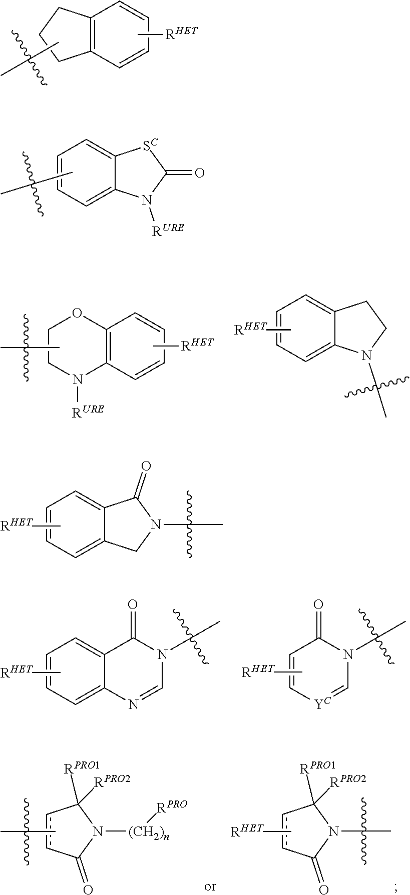

S^{c} of ULM-g through ULM-i is CHR^{SS}, NR^{URE}, or O;
R^{HET} of ULM-g through ULM-i is H, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl);
R^{SS} of ULM-g through ULM-i is H, CN, NO₂, halo (preferably F or Cl), optionally substituted C₁-C₆ alkyl (preferably substituted with one or two hydroxyl groups or up to three halo groups), optionally substituted O—(C₁-C₆ alkyl) (preferably substituted with one or two hydroxyl groups or up to three halo groups) or an optionally substituted —C(O)(C₁-C₆ alkyl) (preferably substituted with one or two hydroxyl groups or up to three halo groups);
R^{URE} of ULM-g through ULM-i is H, a C₁-C₆ alkyl (preferably H or C₁-C₃ alkyl) or a —C(O)(C₁-C₆ alkyl), each of which groups is optionally substituted with one or two hydroxyl groups or up to three halogen, preferably fluorine groups, or an optionally substituted heterocycle, for example piperidine, morpholine, pyrrolidine, tetrahydrofuran, tetrahydrothiophene, piperidine, piperazine, each of which is optionally substituted;
Y^{C} of ULM-g through ULM-i is N or C—R^{YC}, where R^{YC} is H, OH, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl);
R^{PRO} of ULM-g through ULM-i is H, optionally substituted C₁-C₆ alkyl or an optionally substituted aryl (phenyl or napthyl), heteroaryl or heterocyclic group selected from the group consisting of oxazole, isoxazole, thiazole, isothiazole, imidazole, diazole, oximidazole, pyrrole, pyrollidine, furan, dihydrofuran, tetrahydrofuran, thiene, dihydrothiene, tetrahydrothiene, pyridine, piperidine, piperazine, morpholine, quinoline, (each preferably substituted with a C₁-C₃ alkyl group, preferably methyl or a halo group, preferably F or Cl), benzofuran, indole, indolizine, azaindolizine;
R^{PRO1} and R^{PRO2} of ULM-g through ULM-i are each independently H, an optionally substituted C₁-C₃ alkyl group or together form a keto group, and each n of ULM-g through ULM-i is independently 0, 1, 2, 3, 4, 5, or 6 (preferably 0 or 1).

Each of said groups may be optionally connected to a PTM group (including a ULM′ group) via a linker group.

In certain alternative preferred embodiments of the present disclosure, R³ of ULM-g through ULM-i is an optionally substituted —(CH₂)ₙ—(V)_{n′}—(CH₂)ₙ—(V)_{n′}—R^{S3′} group, an optionally substituted-(CH₂)ₙ—N(R_{1′})(C═O)_{m′}—(V)_{n′}—R^{S3′} group, an optionally substituted —X^{R3′}-alkyl group, an optionally substituted —X^{R3′}-Aryl group; an optionally substituted —X^{R3′}-HET group, an optionally substituted —X^{R3′}-Aryl-HET group or an optionally substituted —X^{R3′}-HET-Aryl group,

wherein:
R^{S3} is an optionally substituted alkyl group (C₁-C₁₀, preferably C₁-C₆ alkyl), an optionally substituted Aryl group or a HET group;
R_{1′} is H or a C₁-C₃ alkyl group (preferably H);
V is O, S or NR_{1′};
X^{R3′} is —(CH₂)ₙ—, —(CH₂CH₂O)ₙ—, —CH₂)ₙ—CH(Xᵥ)═CH(Xᵥ)-(cis or trans), —CH₂)ₙ—CH≡CH—, or a C₃-C₆ cycloalkyl group, all optionally substituted;
Xᵥ is H, a halo or a C₁-C₃ alkyl group which is optionally substituted with one or two hydroxyl groups or up to three halogen groups;
Alkyl is an optionally substituted C₁-C₁₀ alkyl (preferably a C₁-C₆ alkyl) group (in certain preferred embodiments, the alkyl group is end-capped with a halo group, often a Cl or Br);
Aryl is an optionally substituted phenyl or napthyl group (preferably, a phenyl group); and
HET is an optionally substituted oxazole, isoxazole, thiazole, isothiazole, imidazole, diazole, oximidazole, pyrrole, pyrollidine, furan, dihydrofuran, tetrahydrofuran, thiene, dihydrothiene, tetrahydrothiene, pyridine, piperidine, piperazine, morpholine, benzofuran, indole, indolizine, azaindolizine, quinoline (when substituted, each preferably substituted with a C₁-C₃ alkyl group, preferably methyl or a halo group, preferably F or Cl), or a group according to the chemical structure:

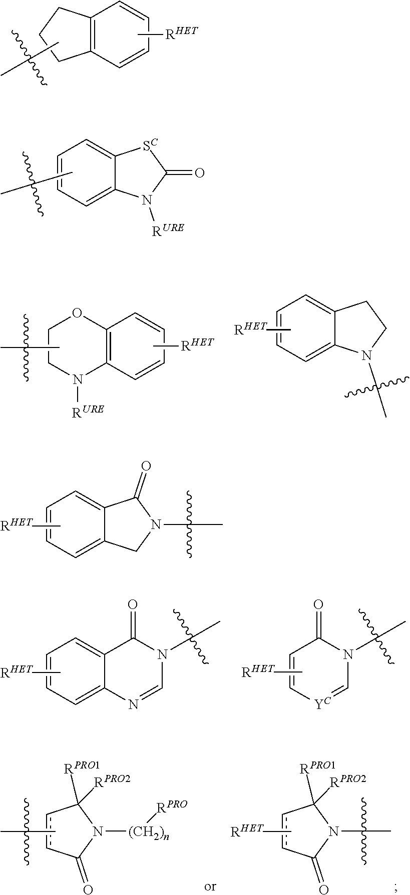

S^{c} of ULM-g through ULM-i is CHR^{SS}, NR^{URE}, or O;
R^{HET} ULM-g through ULM-i is H, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl);
R^{SS} of ULM-g through ULM-i is H, CN, NO₂, halo (preferably F or Cl), optionally substituted C₁-C₆ alkyl (preferably substituted with one or two hydroxyl groups or up to three halo groups), optionally substituted O—(C₁-C₆ alkyl) (preferably substituted with one or two hydroxyl groups or up to three halo groups) or an optionally substituted —C(O)(C₁-C₆ alkyl) (preferably substituted with one or two hydroxyl groups or up to three halo groups);
R^{URE} of ULM-g through ULM-i is H, a C₁-C₆ alkyl (preferably H or C₁-C₃ alkyl) or a —C(O)(C₀-C₆ alkyl), each of which groups is optionally substituted with one or two hydroxyl groups or up to three halogen, preferably fluorine groups, or an optionally substituted heterocycle, for example piperidine, morpholine, pyrrolidine, tetrahydrofuran, tetrahydrothiophene, piperidine, piperazine, each of which is optionally substituted;
Y^{C} of ULM-g through ULM-i is N or C—R^{YC}, where R^{YC} is H, OH, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl);
R^{PRO} of ULM-g through ULM-i is H, optionally substituted C₁-C₆ alkyl or an optionally substituted aryl (phenyl or napthyl), heteroaryl or heterocyclic group selected from the group consisting of oxazole, isoxazole, thiazole, isothiazole, imidazole, diazole, oximidazole, pyrrole, pyrollidine, furan, dihydrofuran, tetrahydrofuran, thiene, dihydrothiene, tetrahydrothiene, pyridine, piperidine, piperazine, morpholine, quinoline, (each preferably substituted with a C₁-C₃ alkyl group, preferably methyl or a halo group, preferably F or Cl), benzofuran, indole, indolizine, azaindolizine;
R^{PRO1} and R^{PRO2} of ULM-g through ULM-i are each independently H, an optionally substituted C₁-C₃ alkyl group or together form a keto group;
each n of ULM-g through ULM-i is independently 0, 1, 2, 3, 4, 5, or 6 (preferably 0 or 1);
each m′ of ULM-g through ULM-i is 0 or 1; and
each n′ of ULM-g through ULM-i is 0 or 1;
wherein each of said compounds, preferably on the alkyl, Aryl or Het groups, is optionally connected to a PTM group (including a ULM′ group) via a linker.

In alternative embodiments, R³′ of ULM-g through ULM-i is —(CH₂)ₙ-Aryl, —(CH₂CH₂O)ₙ-Aryl, —(CH₂)ₙ-HET or —(CH₂CH₂O)ₙ-HET, wherein:
said Aryl of ULM-g through ULM-i is phenyl which is optionally substituted with one or two substitutents, wherein said substituent(s) is preferably selected from —(CH₂)ₙOH, C₁-C₆ alkyl which itself is further optionally substituted with CN, halo (up to three halo groups), OH, —(CH₂)ₙO(C₁-C₆)alkyl, amine, mono- or di-(C₁-C₆ alkyl) amine wherein the alkyl group on the amine is optionally substituted with 1 or 2 hydroxyl groups or up to three halo (preferably F, Cl) groups, or
said Aryl group of ULM-g through ULM-i is substituted with —(CH₂)ₙOH, —(CH₂)ₙ—O—(C₁-C₆)alkyl, —(CH₂)ₙ—O—(CH₂)ₙ—(C₁-C₆)alkyl, —(CH₂)ₙ—C(O)O(C₀-C₆) alkyl, —(CH₂)ₙ—C(O)O(C₀-C₆)alkyl, —(CH₂)ₙ—OC(O)(C₀-C₆)alkyl, amine, mono- or di-(C₁-C₆ alkyl) amine wherein the alkyl group on the amine is optionally substituted with 1 or 2 hydroxyl groups or up to three halo (preferably F, Cl) groups, CN, NO₂, an optionally substituted —(CH₂)ₙ—(V)_{m′}—CH₂)ₙ—(V)_{m′}—(C₁-C₆)alkyl group, a —(V)_{m′}—(CH₂CH₂O)ₙ—R^{PEG} group where V is O, S or NR_{1′}, R_{1′} is H or a C₁-C₃ alkyl group (preferably H) and R^{PEG} is H or a C₁-C₆ alkyl group which is optionally substituted (including being optionally substituted with a carboxyl group), or
said Aryl group of ULM-g through ULM-i is optionally substituted with a heterocycle, including a heteroaryl, selected from the group consisting of oxazole, isoxazole, thiazole, isothiazole, imidazole, diazole, oximidazole, pyrrole, pyrollidine, furan, dihydrofuran, tetrahydrofuran, thiene, dihydrothiene, tetrahydrothiene, pyridine, piperidine, piperazine, morpholine, quinoline, benzofuran, indole, indolizine, azaindolizine, (when substituted each preferably substituted with a C₁-C₃ alkyl group, preferably methyl or a halo group, preferably F or Cl), or a group according to the chemical structure:

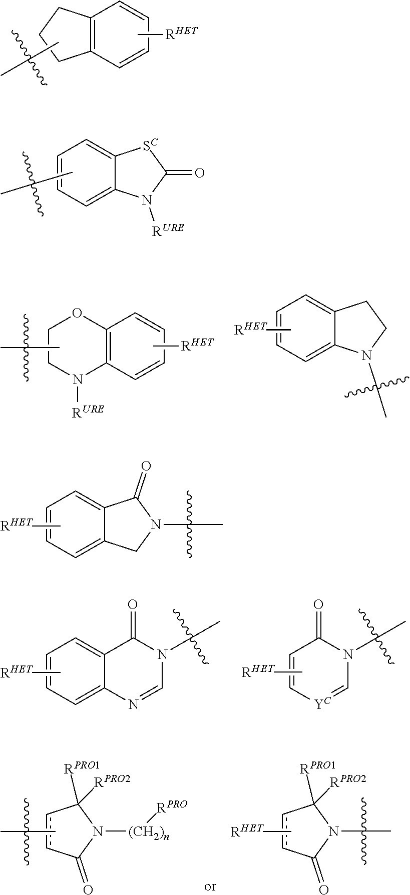

S^{c} of ULM-g through ULM-i is CHR^{SS}, NR^{URE}, or O;
R^{HET} of ULM-g through ULM-i is H, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl);
R^{SS} of ULM-g through ULM-i is H, CN, NO₂, halo (preferably F or Cl), optionally substituted C₁-C₆ alkyl (preferably substituted with one or two hydroxyl groups or up to three halo groups), optionally substituted O—(C₁-C₆ alkyl) (preferably substituted with one or two hydroxyl groups or up to three halo groups) or an optionally substituted —C(O)(C₁-C₆ alkyl) (preferably substituted with one or two hydroxyl groups or up to three halo groups);
R^{URE} of ULM-g through ULM-i is H, a C₁-C₆ alkyl (preferably H or C₁-C₃ alkyl) or a —C(O)(C₀-C₆ alkyl), each of which groups is optionally substituted with one or two hydroxyl groups or up to three halogen, preferably fluorine groups, or an optionally substituted heterocycle, for example piperidine, morpholine, pyrrolidine, tetrahydrofuran, tetrahydrothiophene, piperidine, piperazine, each of which is optionally substituted;
Y^{C} of ULM-g through ULM-i is N or C—R^{YC}, where R^{YC} is H, OH, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl);
R^{PRO} of ULM-g through ULM-i is H, optionally substituted C₁-C₆ alkyl or an optionally substituted aryl (phenyl or napthyl), heteroaryl or heterocyclic group selected from the group consisting of oxazole, isoxazole, thiazole, isothiazole, imidazole, diazole, oximidazole, pyrrole, pyrollidine, furan, dihydrofuran, tetrahydrofuran, thiene, dihydrothiene, tetrahydrothiene, pyridine, piperidine, piperazine, morpholine, quinoline, (each preferably substituted with a C₁-C₃ alkyl group, preferably methyl or a halo group, preferably F or Cl), benzofuran, indole, indolizine, azaindolizine;
R^{PRO1} and R^{PRO2} of ULM-g through ULM-i are each independently H, an optionally substituted C₁-C₃ alkyl group or together form a keto group;
HET of ULM-g through ULM-i is preferably oxazole, isoxazole, thiazole, isothiazole, imidazole, diazole, oximidazole, pyrrole, pyrollidine, furan, dihydrofuran, tetrahydrofuran, thiene, dihydrothiene, tetrahydrothiene, pyridine, piperidine, piperazine, morpholine, quinoline, (each preferably substituted with a C₁-C₃ alkyl group, preferably methyl or a halo group, preferably F or Cl), benzofuran, indole, indolizine, azaindolizine, or a group according to the chemical structure:

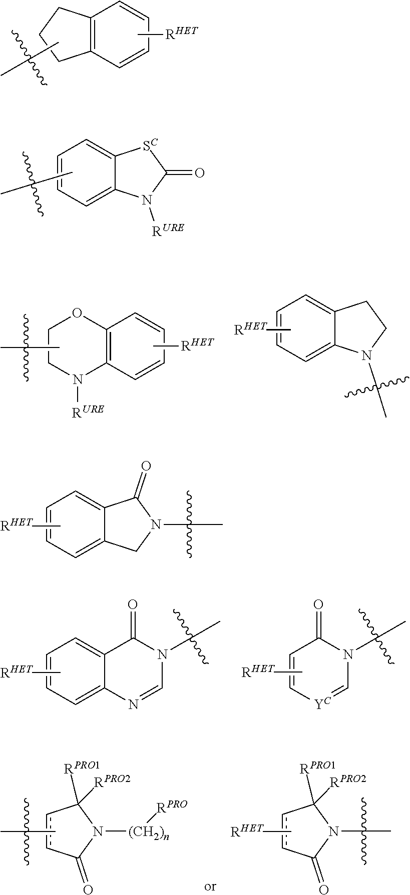

S^{c} of ULM-g through ULM-i is CHR^{SS}, NR^{URE}, or O;
R^{HET} ULM-g through ULM-i is H, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl);
R^{SS} of ULM-g through ULM-i is H, CN, NO₂, halo (preferably F or Cl), optionally substituted C₁-C₆ alkyl (preferably substituted with one or two hydroxyl groups or up to three halo groups), optionally substituted O—(C₁-C₆ alkyl) (preferably substituted with one or two hydroxyl groups or up to three halo groups) or an optionally substituted —C(O)(C₁-C₆ alkyl) (preferably substituted with one or two hydroxyl groups or up to three halo groups);
R^{URE} of ULM-g through ULM-i is H, a C₁-C₆ alkyl (preferably H or C₁-C₃ alkyl) or a —C(O)(C₀-C₆ alkyl), each of which groups is optionally substituted with one or two hydroxyl groups or up to three halogen, preferably fluorine groups, or an optionally substituted heterocycle, for example piperidine, morpholine, pyrrolidine, tetrahydrofuran, tetrahydrothiophene, piperidine, piperazine, each of which is optionally substituted;
Y^{C} of ULM-g through ULM-i is N or C—R^{YC}, where R^{YC} is H, OH, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl);
R^{PRO} of ULM-g through ULM-i is H, optionally substituted C₁-C₆ alkyl or an optionally substituted aryl, heteroaryl or heterocyclic group;
R^{PRO1} and R^{PRO2} of ULM-g through ULM-i are each independently H, an optionally substituted C₁-C₃ alkyl group or together form a keto group;
each m′ of ULM-g through ULM-i is independently 0 or 1; and
each n of ULM-g through ULM-i is independently 0, 1, 2, 3, 4, 5, or 6 (preferably 0 or 1),
wherein each of said compounds, preferably on said Aryl or HET groups, is optionally connected to a PTM group (including a ULM′ group) via a linker group.

In still additional embodiments, preferred compounds include those according to the chemical structure:

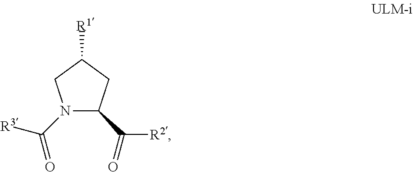

wherein:
R^{1′} of ULM-i is OH or a group which is metabolized in a patient or subject to OH;
R^{2′} of ULM-i is a —NH—CH₂-Aryl-HET (preferably, a phenyl linked directly to a methyl substituted thiazole);
R^{3′} of ULM-i is a —CHR^{CR3′}—NH—C(O)—R^{3P1} group or a —CHR^{CR3′}—R^{3P2} group;
R^{CR3′} of ULM-i is a C₁-C₄ alkyl group, preferably methyl, isopropyl or tert-butyl;
R^{3P1} of ULM-i is C₁-C₃ alkyl (preferably methyl), an optionally substituted oxetane group (preferably methyl substituted, a —(CH₂)ₙOCH₃ group where n is 1 or 2 (preferably 2), or
   a

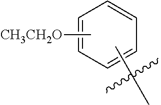

group (the ethyl ether group is preferably meta-substituted on the phenyl moiety), a morpholino group (linked to the carbonyl at the 2- or 3-position;
R^{3P2} of ULM-i is a

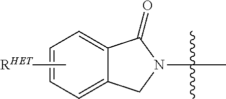

group;
Aryl of ULM-i is phenyl;
HET of ULM-i is an optionally substituted thiazole or isothiazole; and
R^{HET} of ULM-i is H or a halo group (preferably H);
or a pharmaceutically acceptable salt, stereoisomer, solvate or polymorph thereof, wherein each of said compounds is optionally connected to a PTM group (including a ULM′ group) via a linker group.

In certain aspects, bifunctional compounds comprising a ubiquitin E3 ligase binding moiety (ULM), wherein ULM is a group according to the chemical structure:

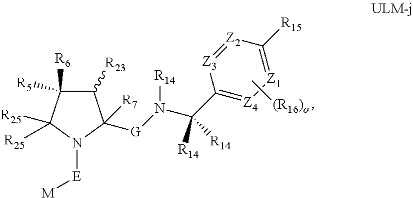

wherein:
each R₅ and R₆ of ULM-j is independently OH, SH, or optionally substituted alkyl or R₅, R₆, and the carbon atom to which they are attached form a carbonyl;
R₇ of ULM-j is H or optionally substituted alkyl;
E of ULM-j is a bond, C═O, or C═S;
G of ULM-j is a bond, optionally substituted alkyl, —COOH or C=J;
J of ULM-j is O or N—R₈;
R₈ of ULM-j is H, CN, optionally substituted alkyl or optionally substituted alkoxy;
M of ULM-j is optionally substituted aryl, optionally substituted heteroaryl, optionally substituted heterocyclic or

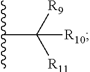

each R₉ and R₁₀ of ULM-j is independently H; optionally substituted alkyl, optionally substituted cycloalkyl, optionally substituted hydroxyalkyl, optionally substituted thioalkyl, a disulphide linked ULM, optionally substituted heteroaryl, or haloalkyl; or R₉, R₁₀, and the carbon atom to which they are attached form an optionally substituted cycloalkyl;
R₁₁ of ULM-j is optionally substituted heterocyclic, optionally substituted alkoxy, optionally substituted heteroaryl, optionally substituted aryl, or

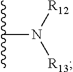

R₁₂ of ULM-j is H or optionally substituted alkyl;
R₁₃ of ULM-j is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl; optionally substituted (oxoalkyl)carbamate,
each R₁₄ of ULM-j is independently H, haloalkyl, optionally substituted cycloalkyl, optionally substituted alkyl or optionally substituted heterocycloalkyl;
R₁₅ of ULM-j is H, optionally substituted heteroaryl, haloalkyl, optionally substituted aryl, optionally substituted alkoxy, or optionally substituted heterocyclyl;
each R₁₆ of ULM-j is independently halo, optionally substituted alkyl, optionally substituted haloalkyl, CN, or optionally substituted haloalkoxy;
each R₂₅ of ULM-j is independently H or optionally substituted alkyl; or both R₂₅ groups can be taken together to form an oxo or optionally substituted cycloalkyl group;
R₂₃ of ULM-j is H or OH;
Z₁, Z₂, Z₃, and Z₄ of ULM-j are independently C or N; and
o of ULM-j is 0, 1, 2, 3, or 4, or a pharmaceutically acceptable salt, stereoisomer, solvate or polymorph thereof.

In certain embodiments, wherein G of ULM-j is C=J, J is O, R₇ is H, each R₁₄ is H, and o is 0.

In certain embodiments, wherein G of ULM-j is C=J, J is O, R₇ is H, each R₁₄ is H, R₁₅ is optionally substituted heteroaryl, and o is 0. In other instances, E is C═O and M is

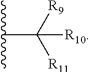

In certain embodiments, wherein E of ULM-j is C═O, R₁₁ is optionally substituted heterocyclic or

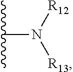

### and M is

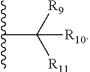

In certain embodiments, wherein E of ULM-j is C═O, M is

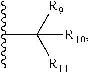

### and R₁₁ is

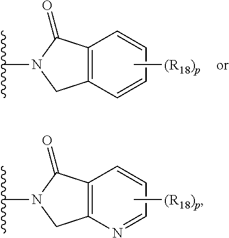

each R₁₈ is independently halo, optionally substituted alkoxy, cyano, optionally substituted alkyl, haloalkyl, or haloalkoxy; and p is 0, 1, 2, 3, or 4.

In certain embodiments, ULM and where present, ULM′, are each independently a group according to the chemical structure:

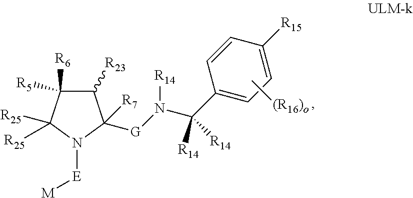

wherein:
G of ULM-k is C=J, J is O;
R₇ of ULM-k is H;
each R₁₄ of ULM-k is H;
o of ULM-k is 0;
R₁₅ of ULM-k is

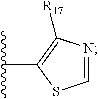

and
R₁₇ of ULM-k is H, halo, optionally substituted cycloalkyl, optionally substituted alkyl, optionally substituted alkenyl, and haloalkyl.

In other instances, R₁₇ of ULM-k is alkyl (e.g., methyl) or cycloalkyl (e.g., cyclopropyl).

In other embodiments, ULM and where present, ULM′, are each independently a group according to the chemical structure:

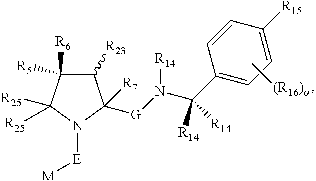

wherein:
G of ULM-k is C=J, J is O;
R₇ of ULM-k is H;
each R₁₄ of ULM-k is H;
o of ULM-k is 0; and

R₁₅ of ULM-k is selected from the group consisting of:

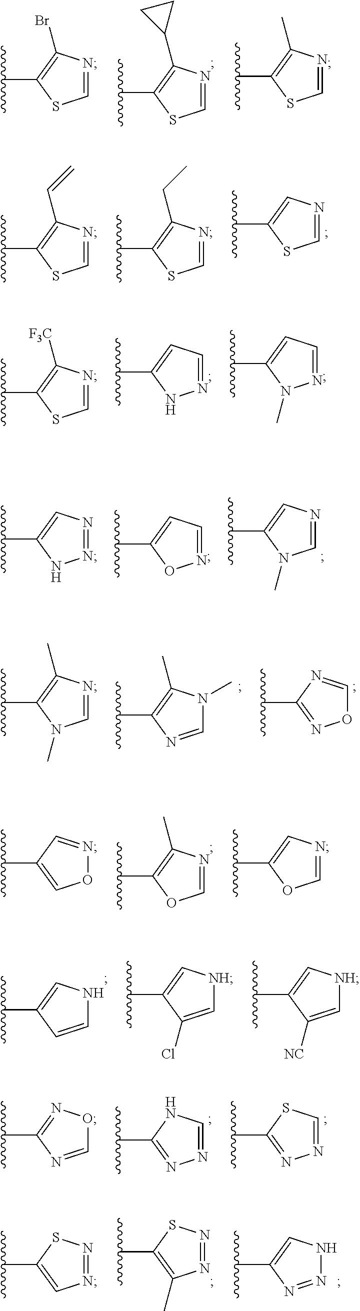

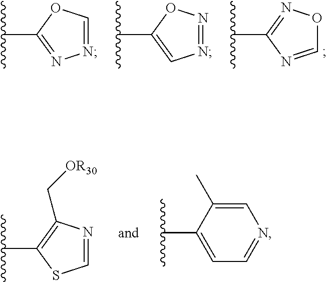

wherein R₃₀ of ULM-k is H or an optionally substituted alkyl.

In other embodiments, ULM and where present, ULM′, are each independently a group according to the chemical structure:

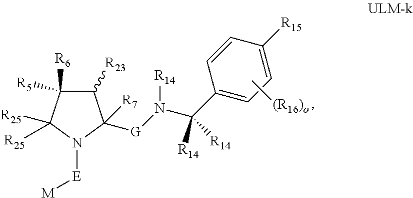

wherein:
E of ULM-k is C═O;
M of ULM-k is

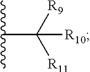

and
R₁₁ of ULM-k is selected from the group consisting of:

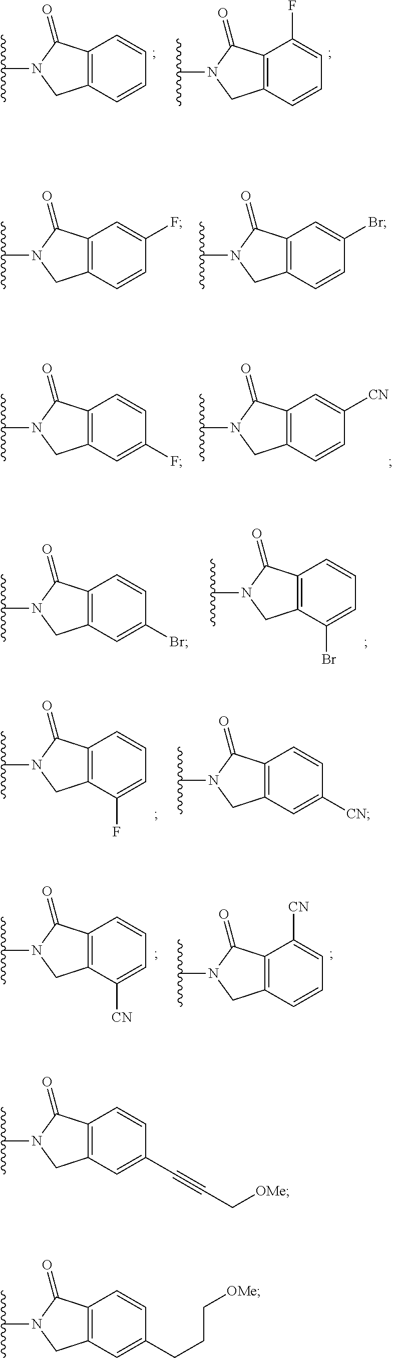

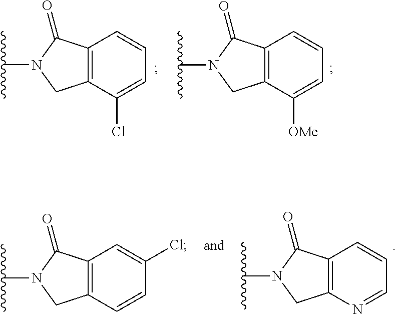

In still other embodiments, a compound of the chemical structure,

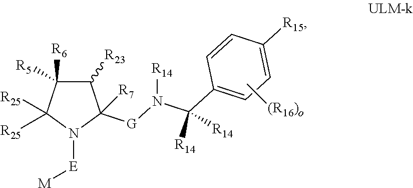

wherein E of ULM-k is C═O;

### R₁₁ of ULM-k is

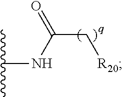

and

### M of ULM-k is

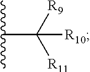

### q of ULM-k is 1 or 2;

R₂₀ of ULM-k is H, optionally substituted alkyl, optionally substituted cycloalkyl, optionally substituted aryl, or

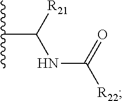

R₂₁ of ULM-k is H or optionally substituted alkyl; and
R₂₂ of ULM-k is H, optionally substituted alkyl, optionally substituted alkoxy, or haloalkyl.

In any embodiment described herein, R₁₁ of ULM-j or ULM-k is selected from the group consisting of:

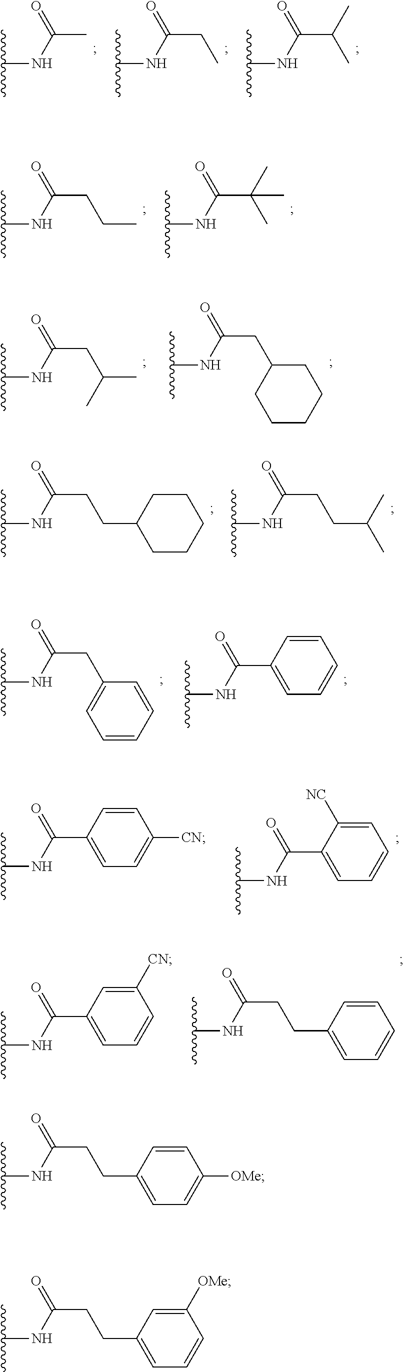

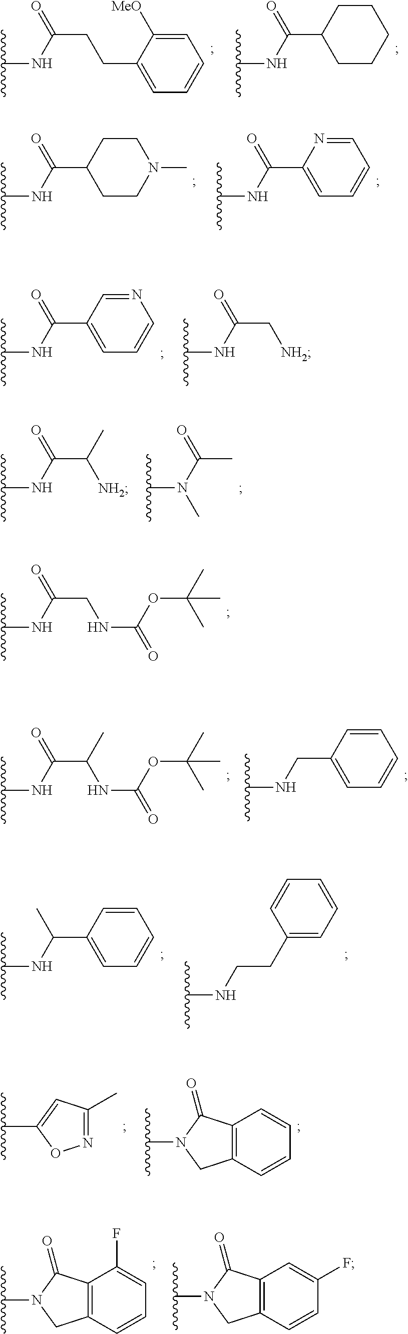

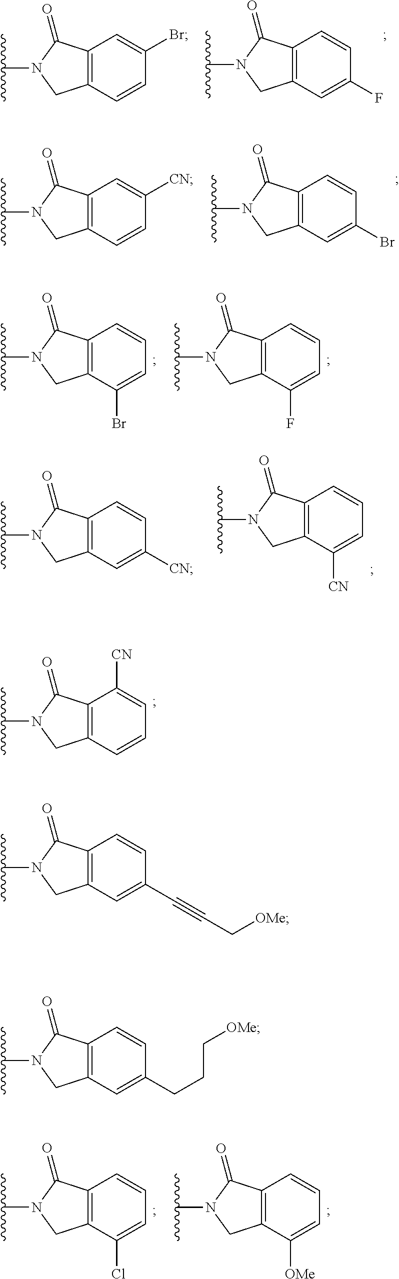

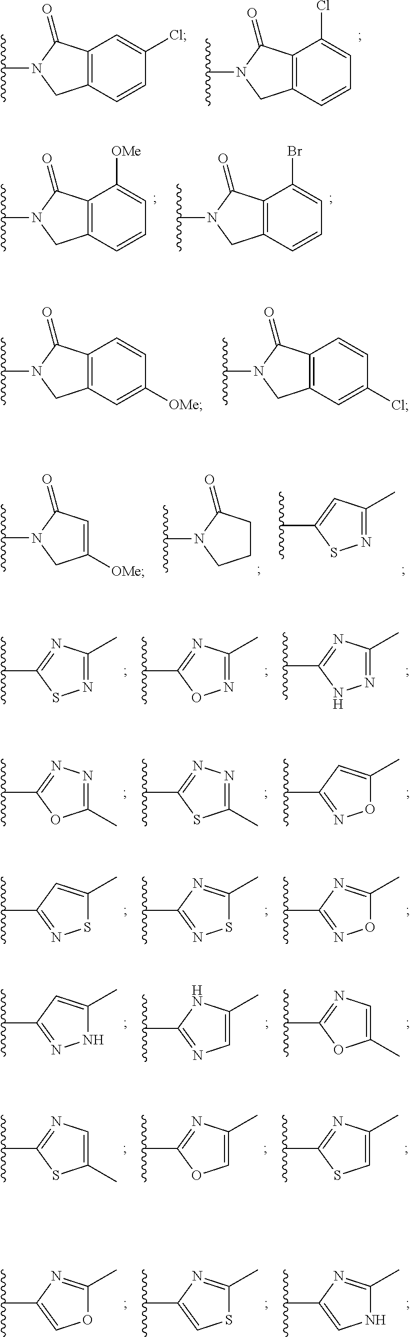

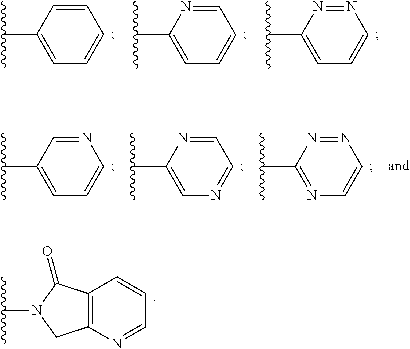

In certain embodiments, R₁₁ of ULM-j or ULM-k is selected from the group consisting of:

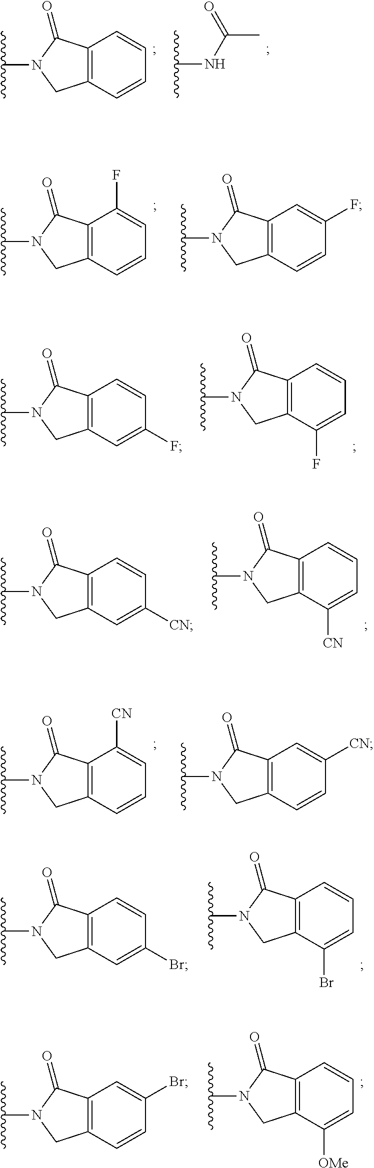

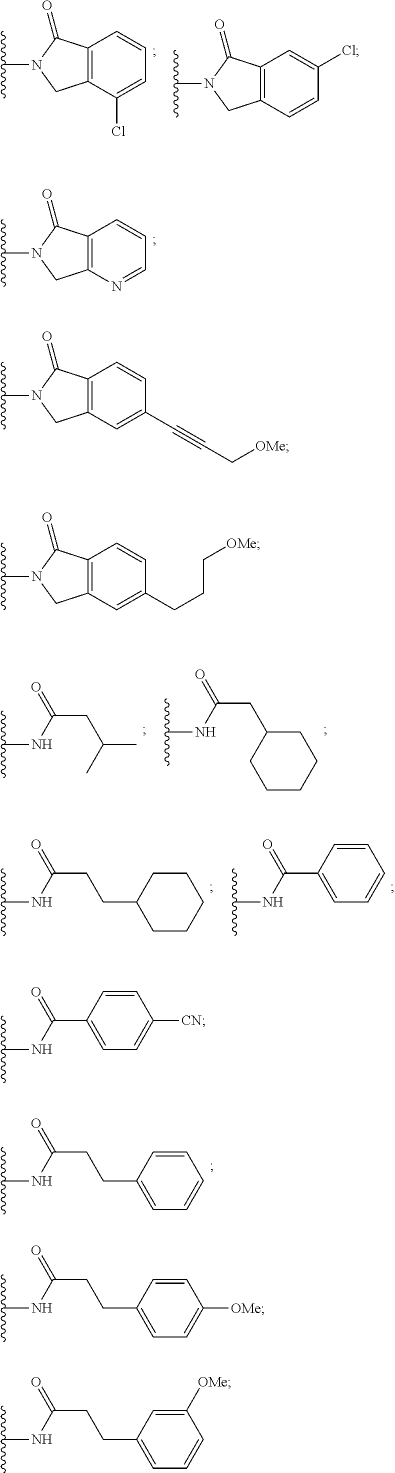

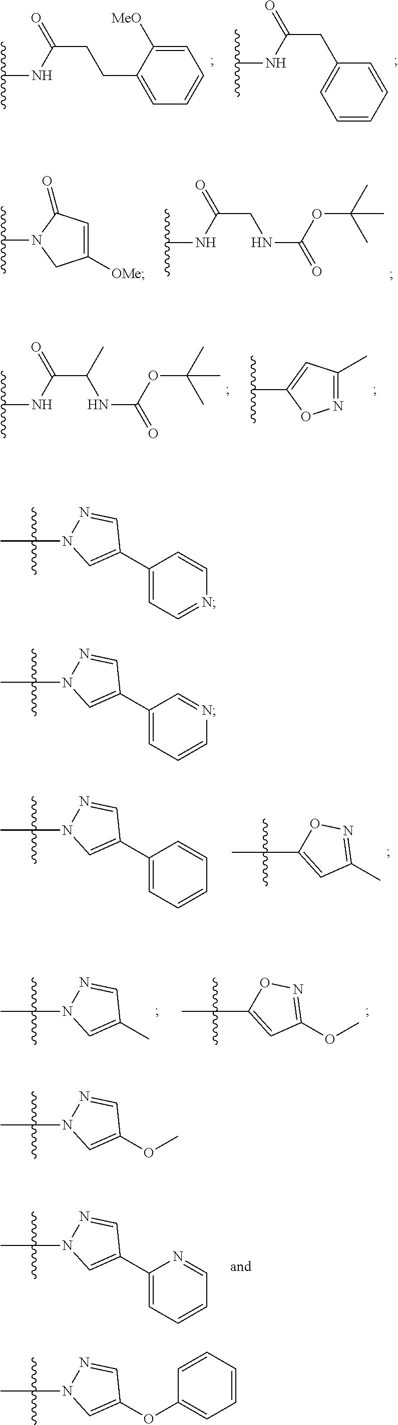

In certain embodiments, ULM (or when present ULM′) is a group according to the chemical structure:

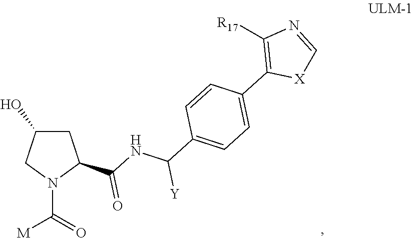

wherein:
X of ULM-1 is O or S;
Y of ULM-1 is H, methyl or ethyl;
R₁₇ of ULM-1 is H, methyl, ethyl, hydroxymethyl or cyclopropyl;
M of ULM-1 is is optionally substituted aryl, optionally substituted heteroaryl, or

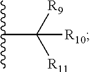

R₉ of ULM-i is H;
R₁₀ of ULM-1 is H, optionally substituted alkyl, optionally substituted haloalkyl, optionally substituted heteroaryl, optionally substituted aryl, optionally substituted hydroxyalkyl, optionally substituted thioalkyl or cycloalkyl;
R11 of ULM-1 is optionally substituted heteroaromatic, optionally substituted heterocyclic, optionally substituted aryl or

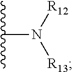

R₁₂ of ULM-1 is H or optionally substituted alkyl; and
R₁₃ of ULM-1 is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl; optionally substituted (oxoalkyl)carbamate.

In some embodiments, ULM and where present, ULM′, are each independently a group according to the chemical structure:

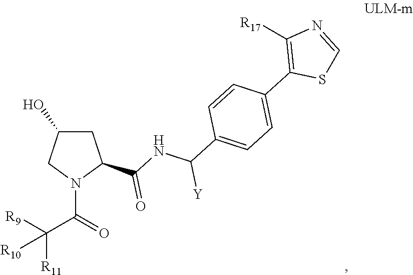

wherein:
Y of ULM-m is H, methyol or ethyl
R₉ ULM-m is H;
R₁₀ is isopropyl, tert-butyl, sec-butyl, cyclopentyl, or cyclohexyl;
R₁₁ of ULM-m is optionally substituted amide, optionally substituted isoindolinone, optionally substituted isooxazole, optionally substituted heterocycles.

In other preferred embodiments of the disclosure, ULM and where present, ULM′, are each independently a group according to the chemical structure:

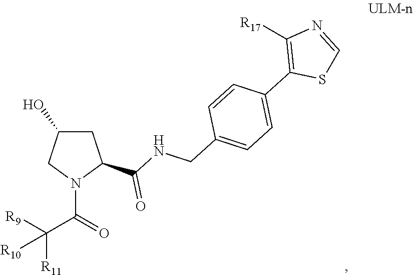

wherein:
R₁₇ of ULM-n is methyl, ethyl, or cyclopropyl; and
R₉, R₁₀, and R₁₁ of ULM-n are as defined above. In other instances, R₉ is H; and
R₁₀ of ULM-n is H, alkyl, or or cycloalkyl (preferably, isopropyl, tert-butyl, sec-butyl, cyclopentyl, or cyclohexyl).

In any of the aspects or embodiments described herein, the ULM (or when present, ULM′) as described herein may be a pharmaceutically acceptable salt, enantiomer, diastereomer, solvate or polymorph thereof. In addition, in any of the aspects or embodiments described herein, the ULM (or when present, ULM′) as described herein may be coupled to a PTM directly via a bond or by a chemical linker.

In certain aspects of the disclosure, the ULM moiety is selected from the group consisting of:

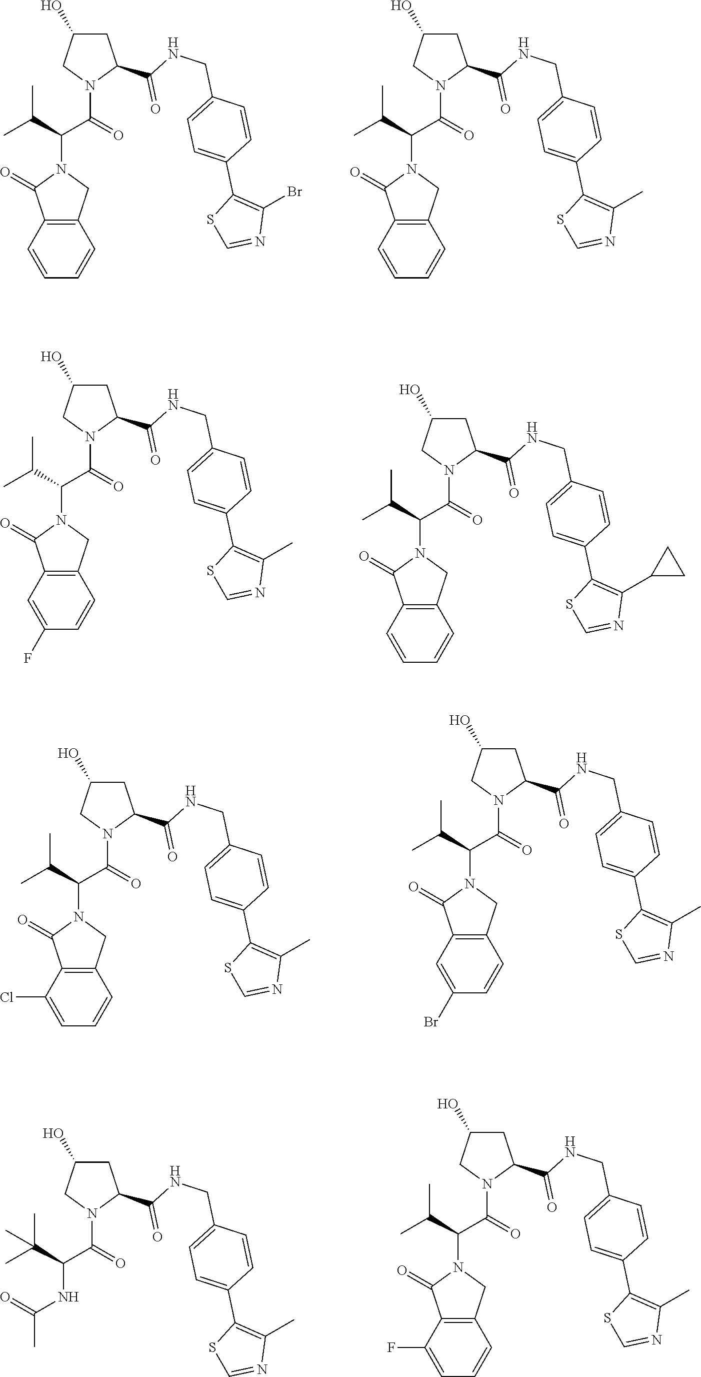

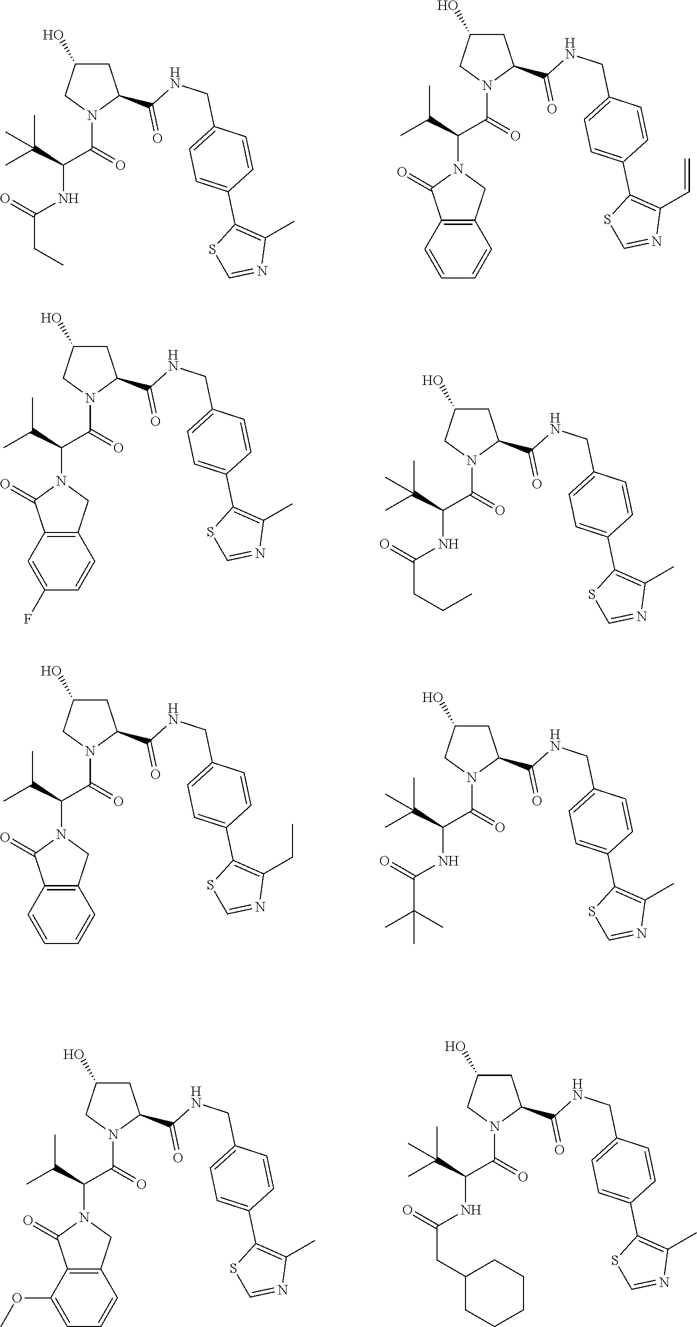

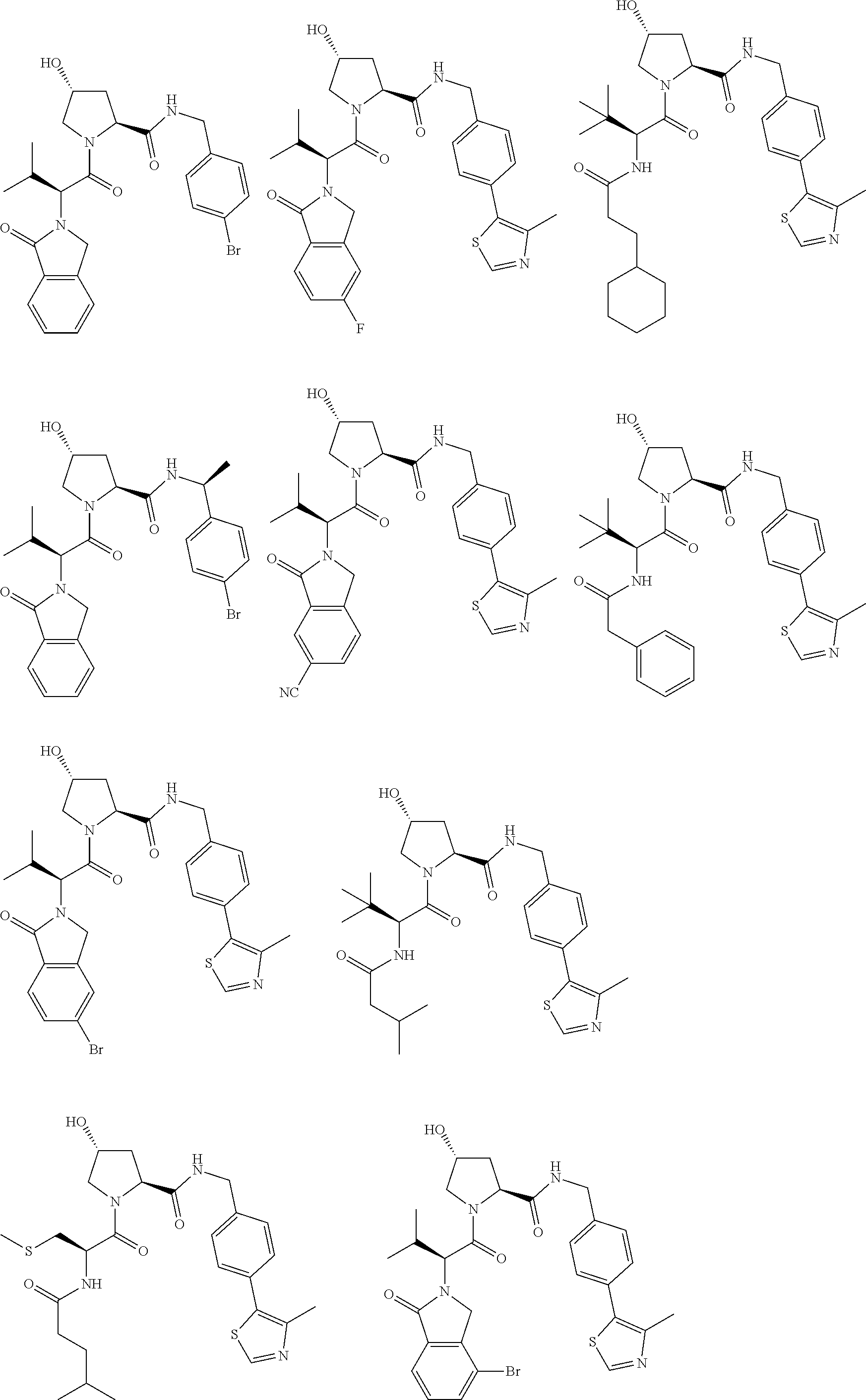

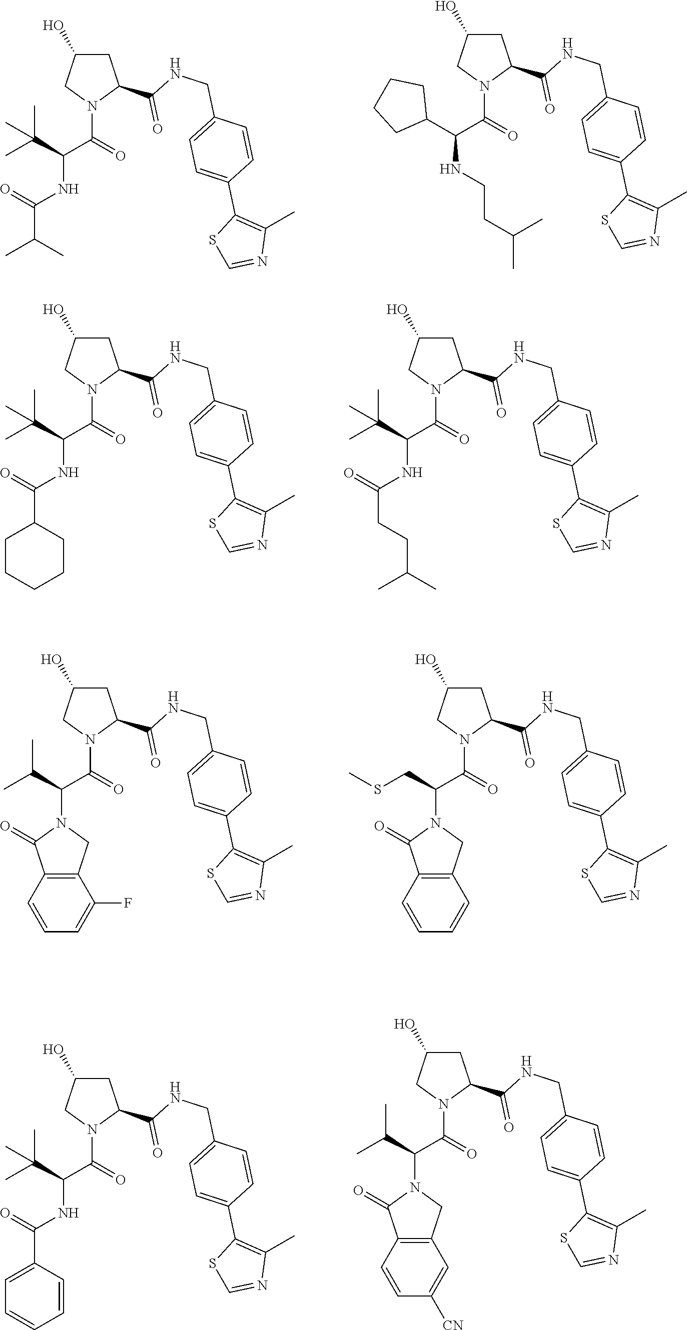

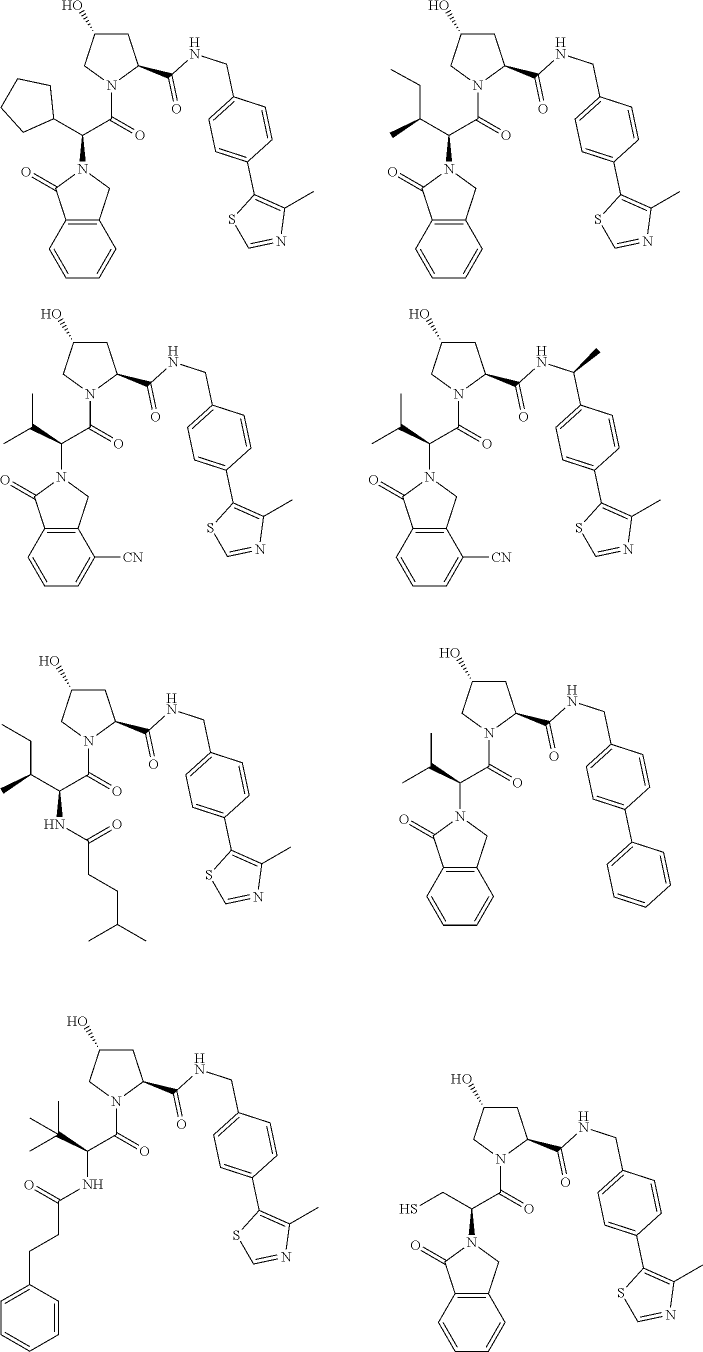

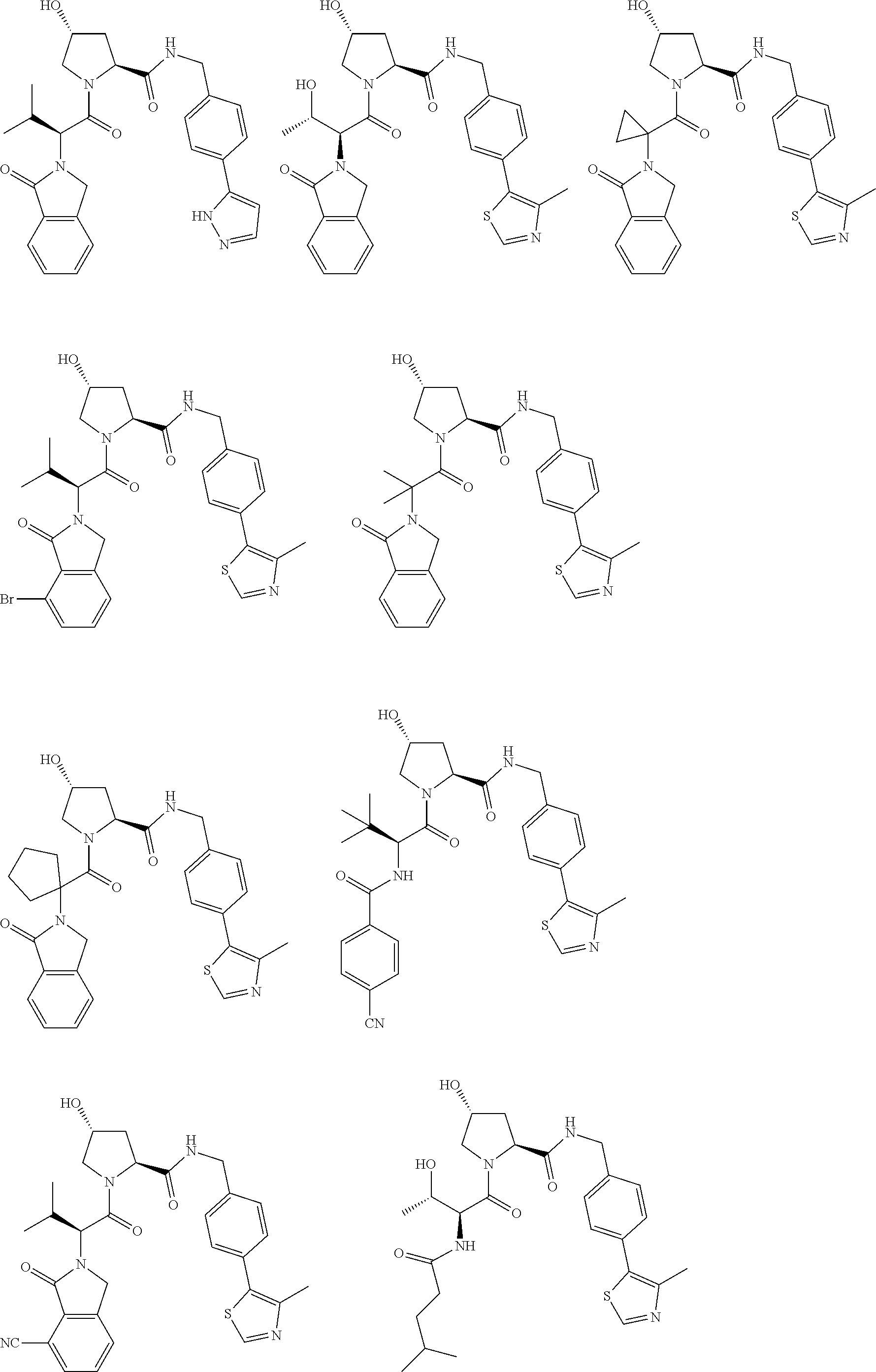

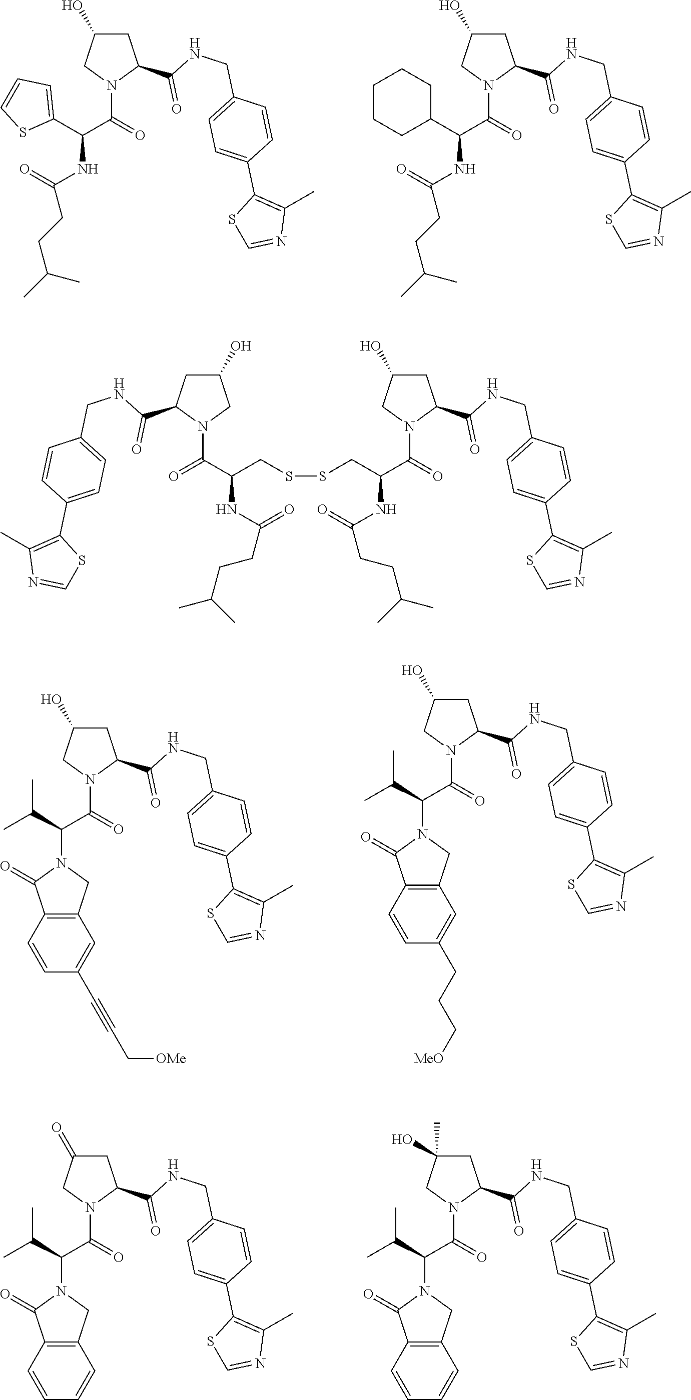

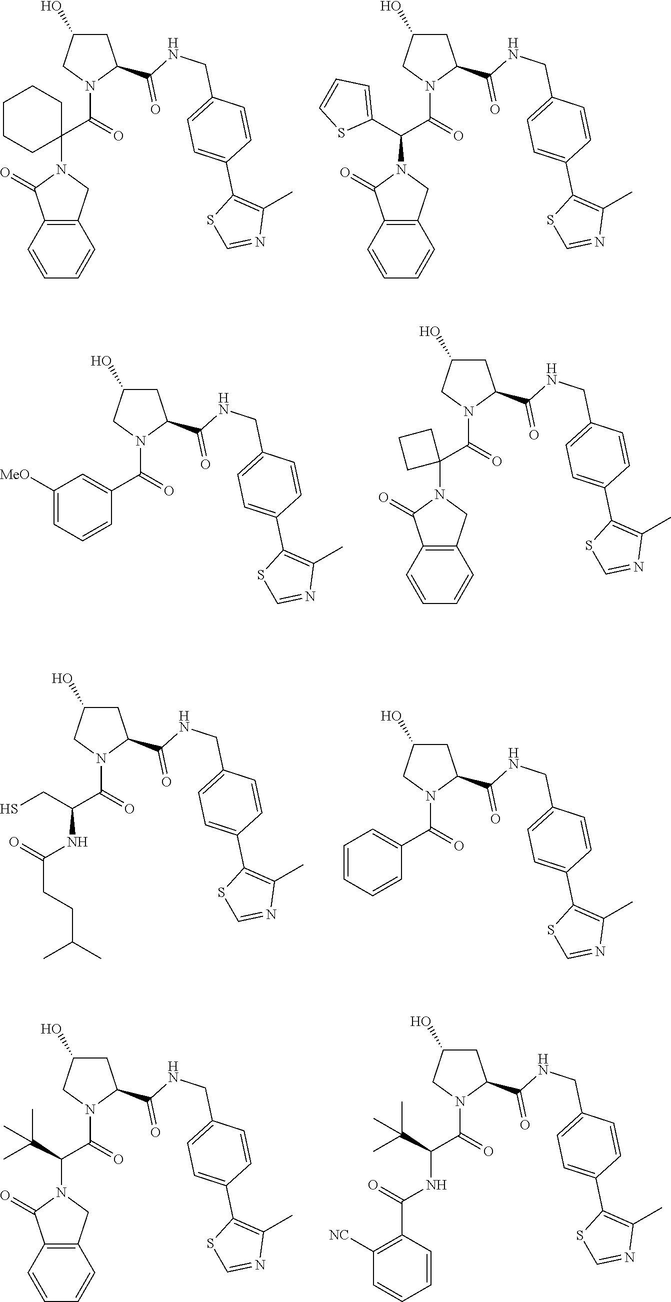

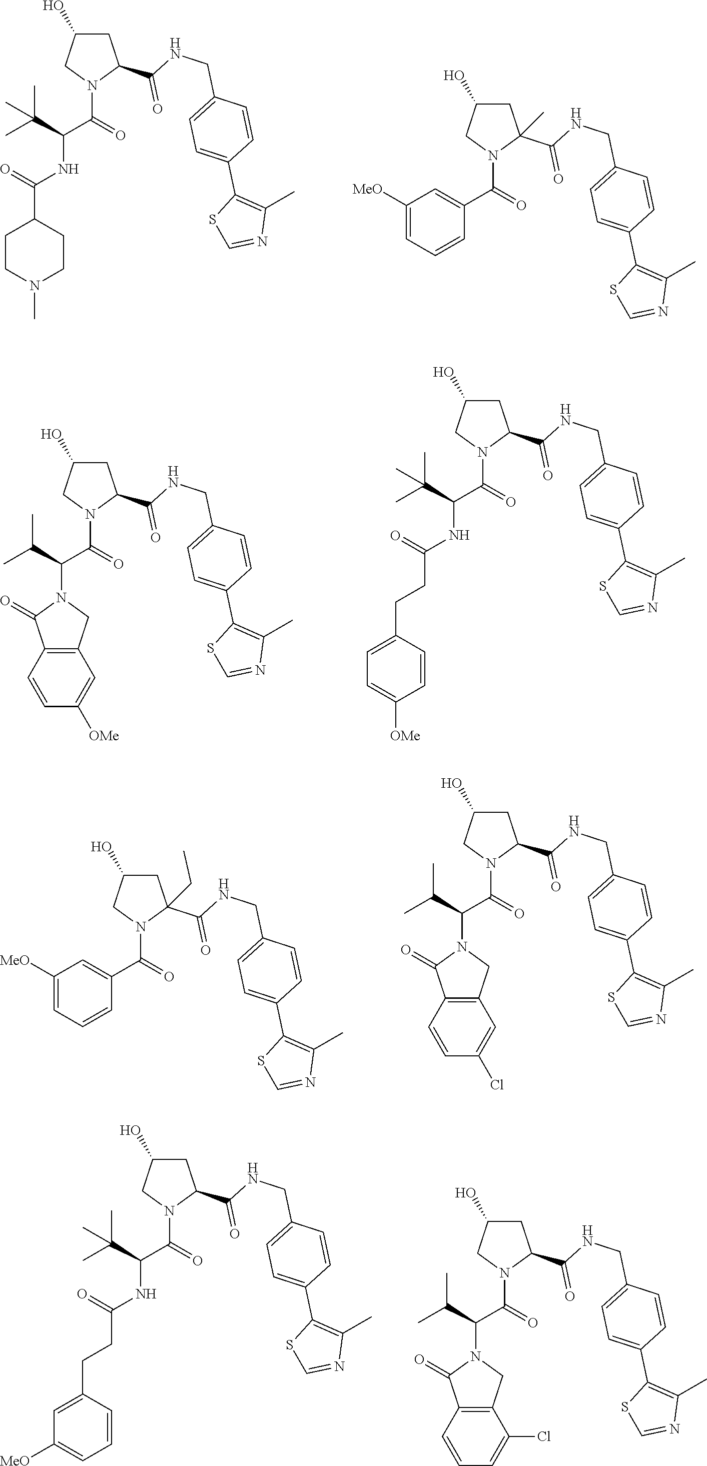

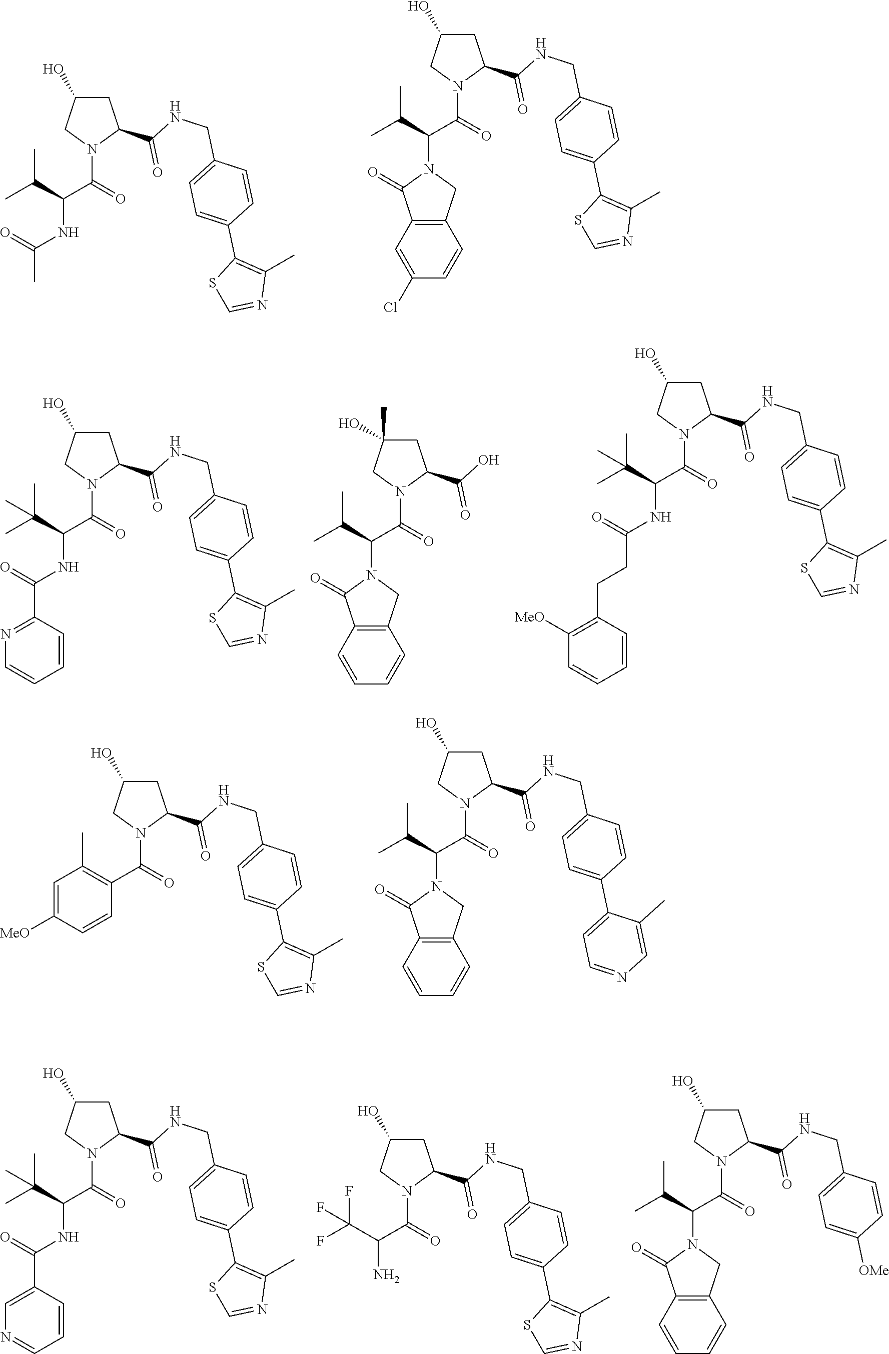

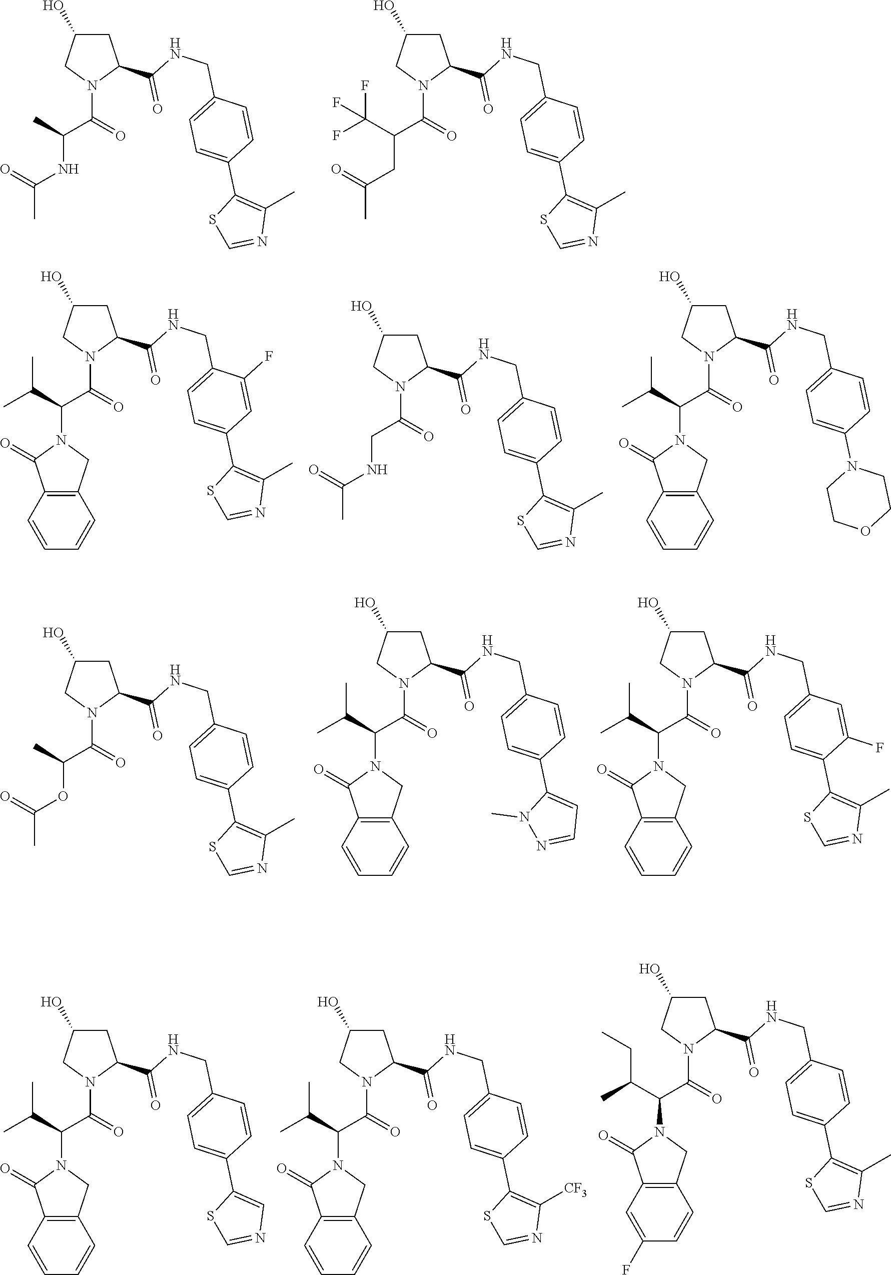

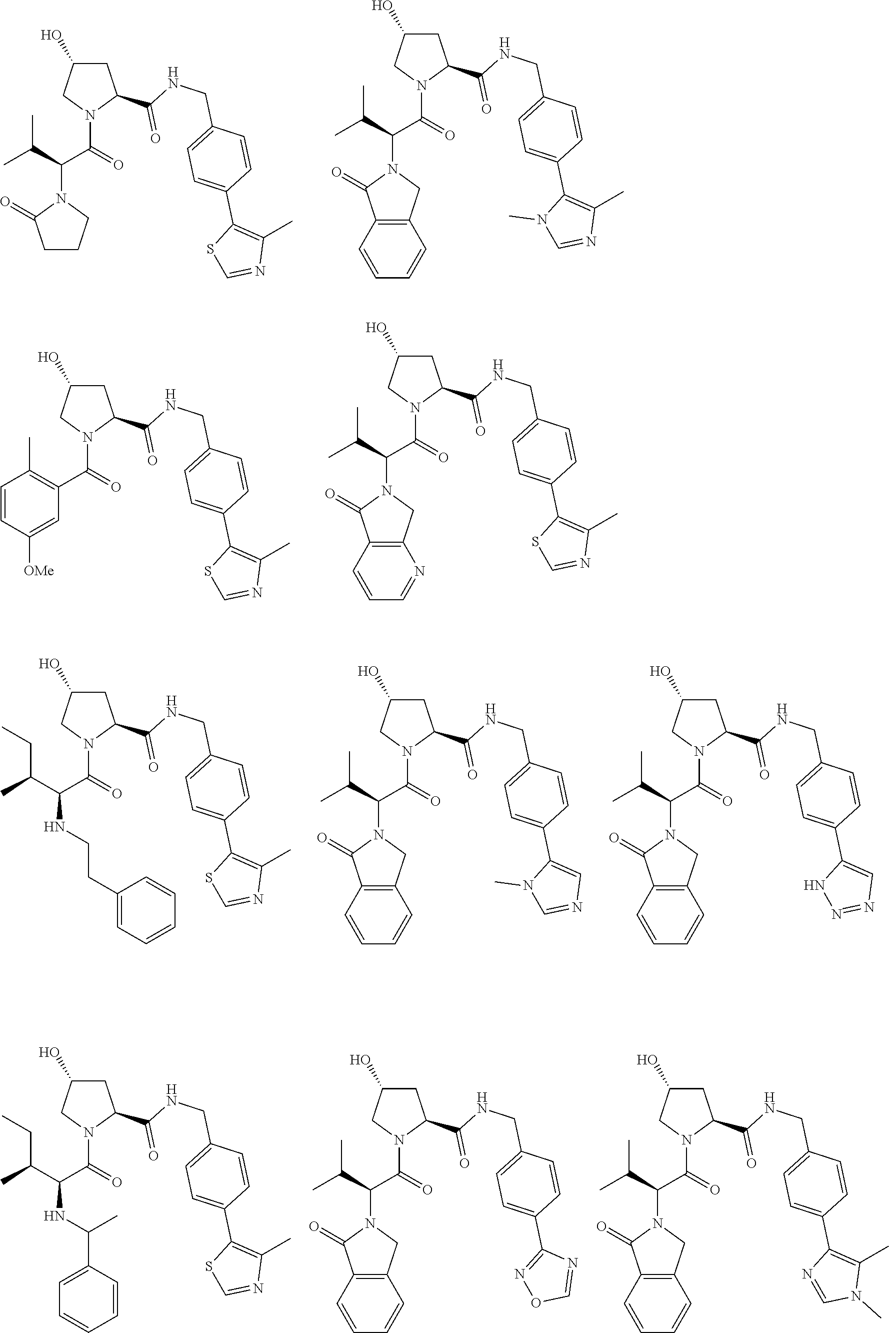

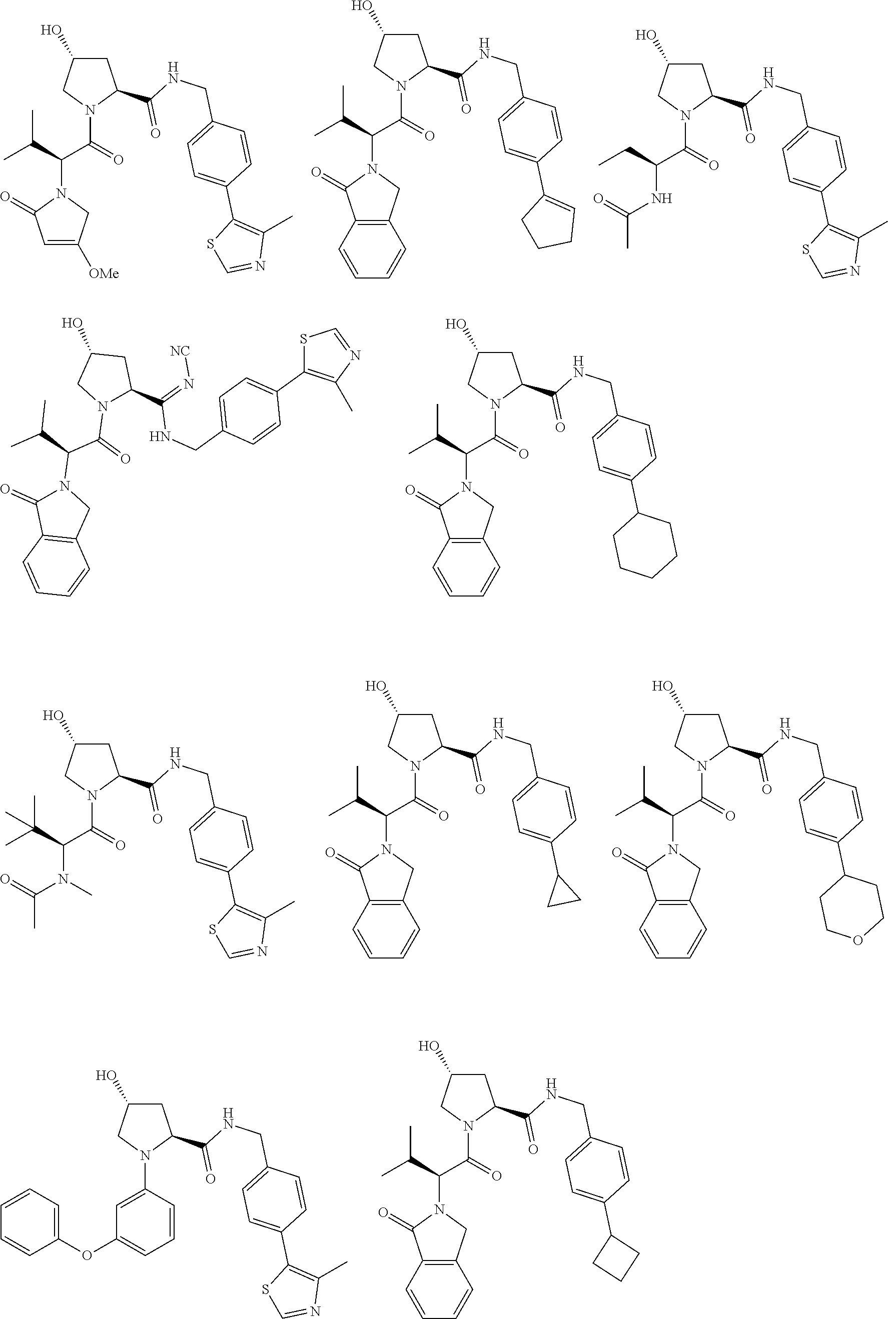

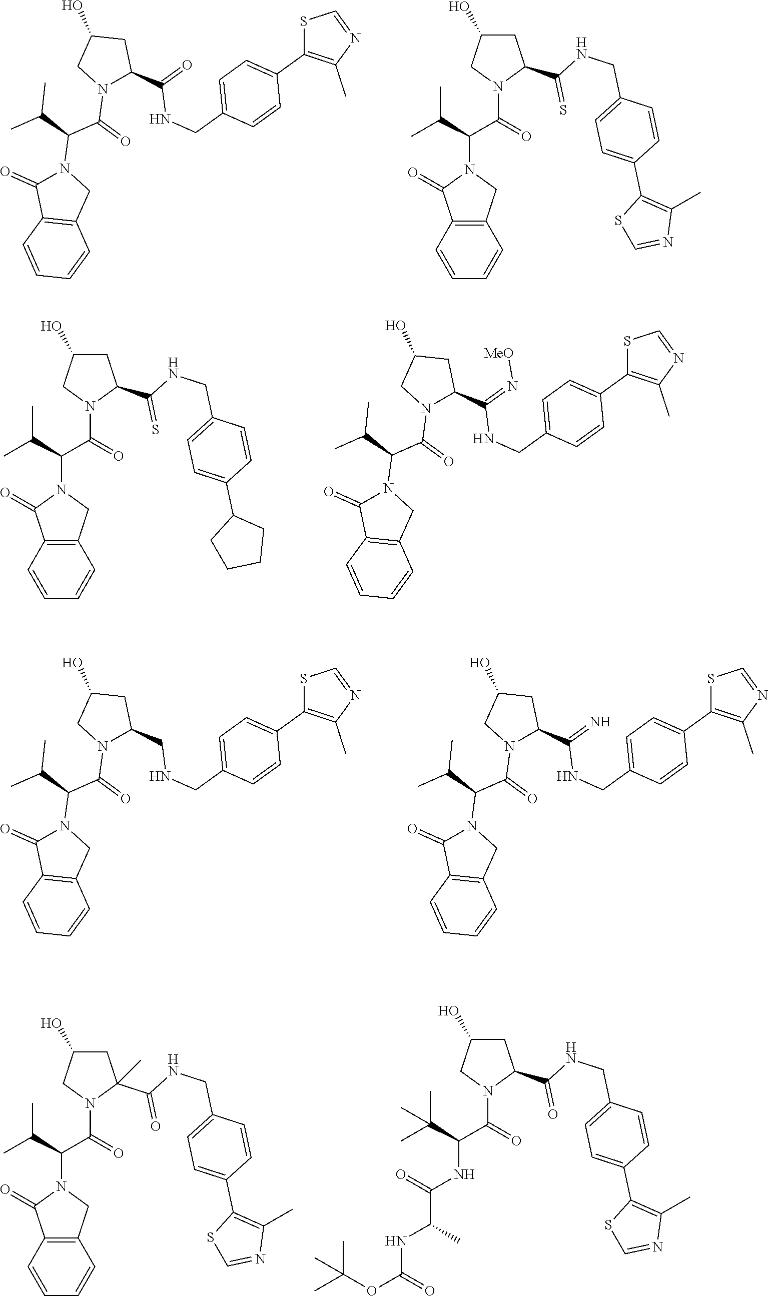

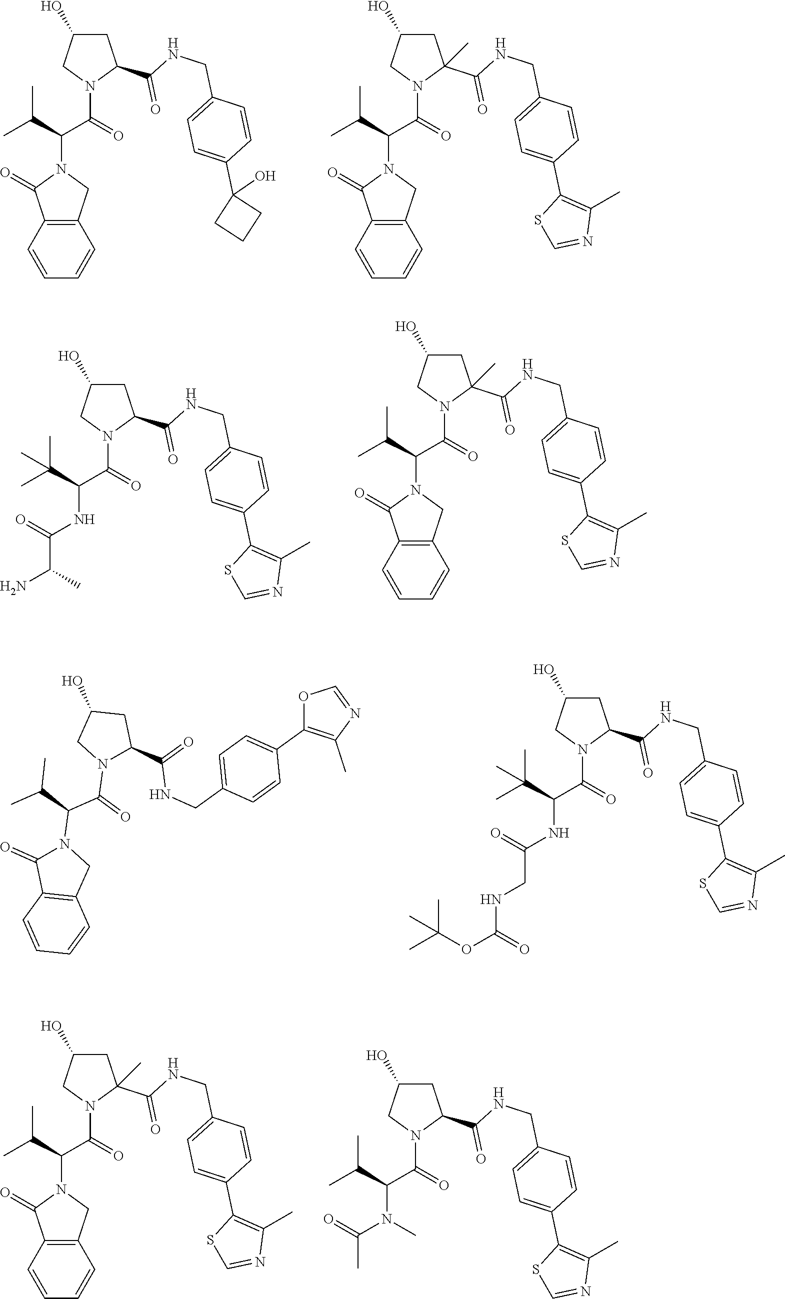

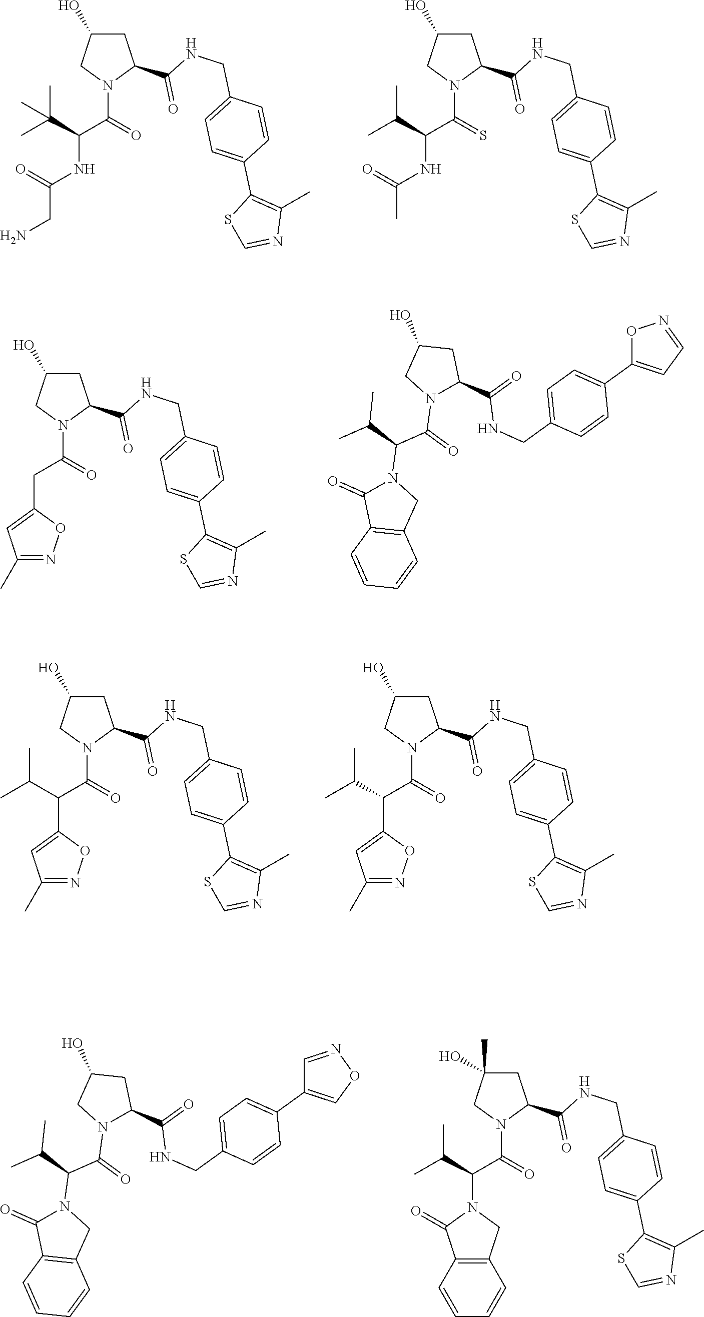

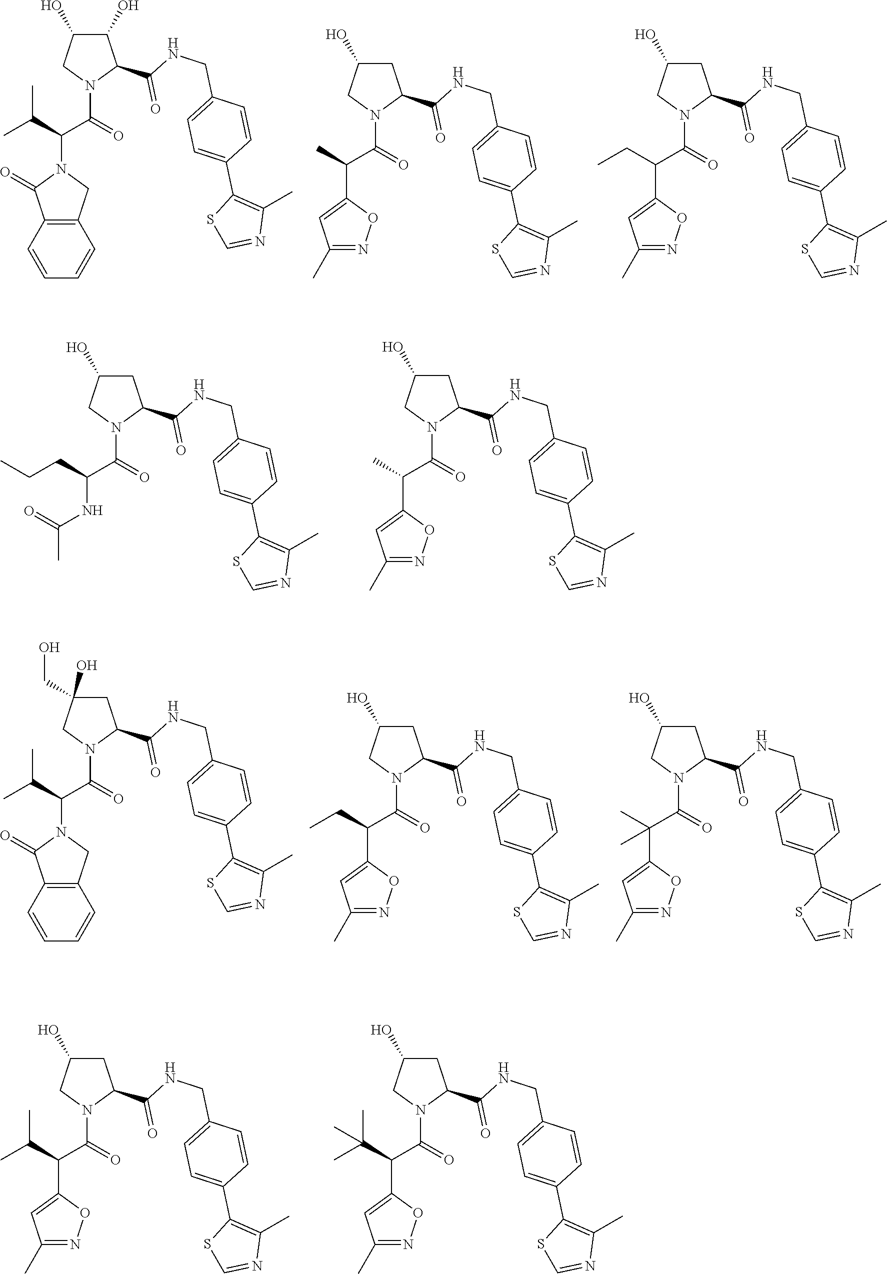

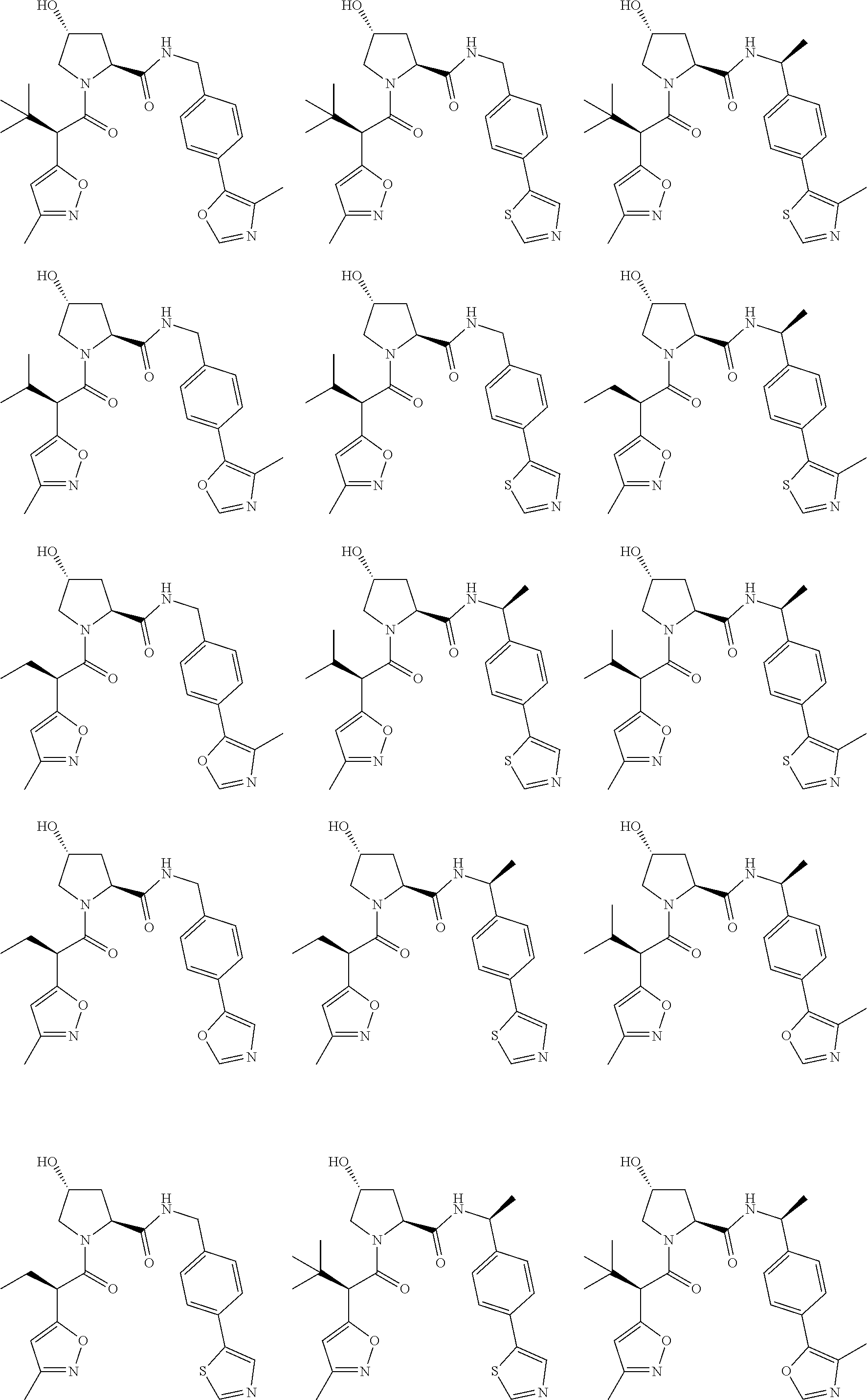

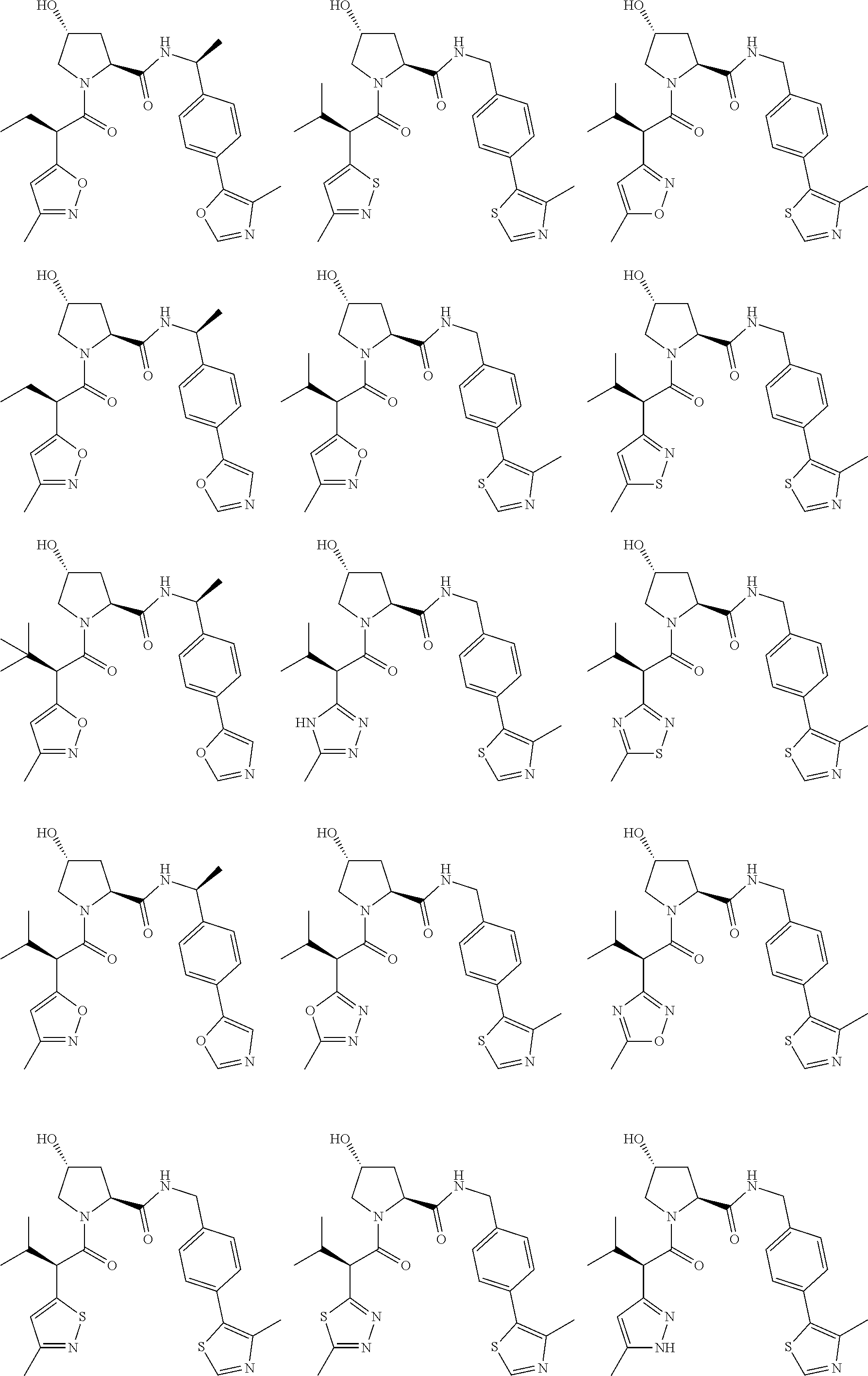

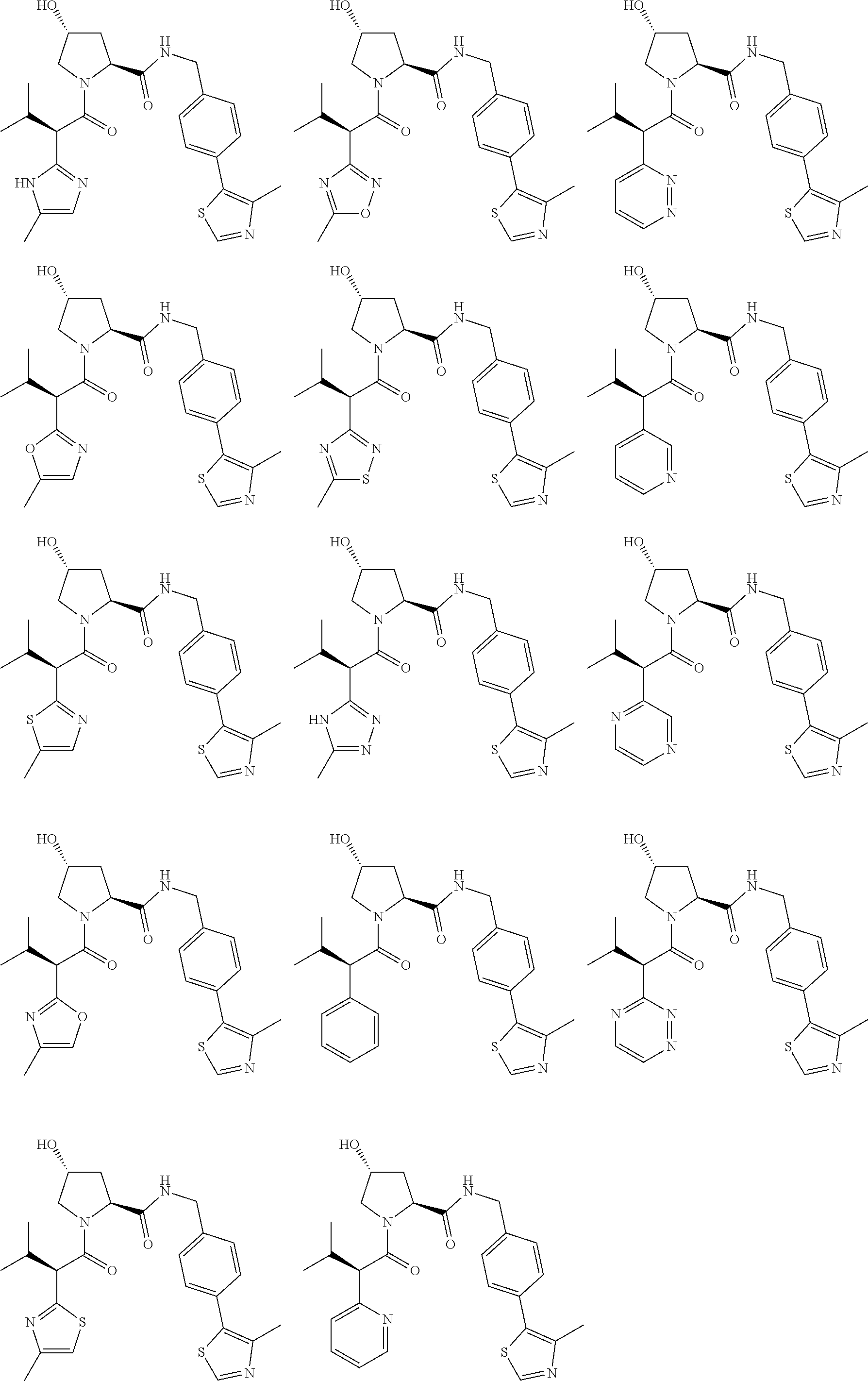

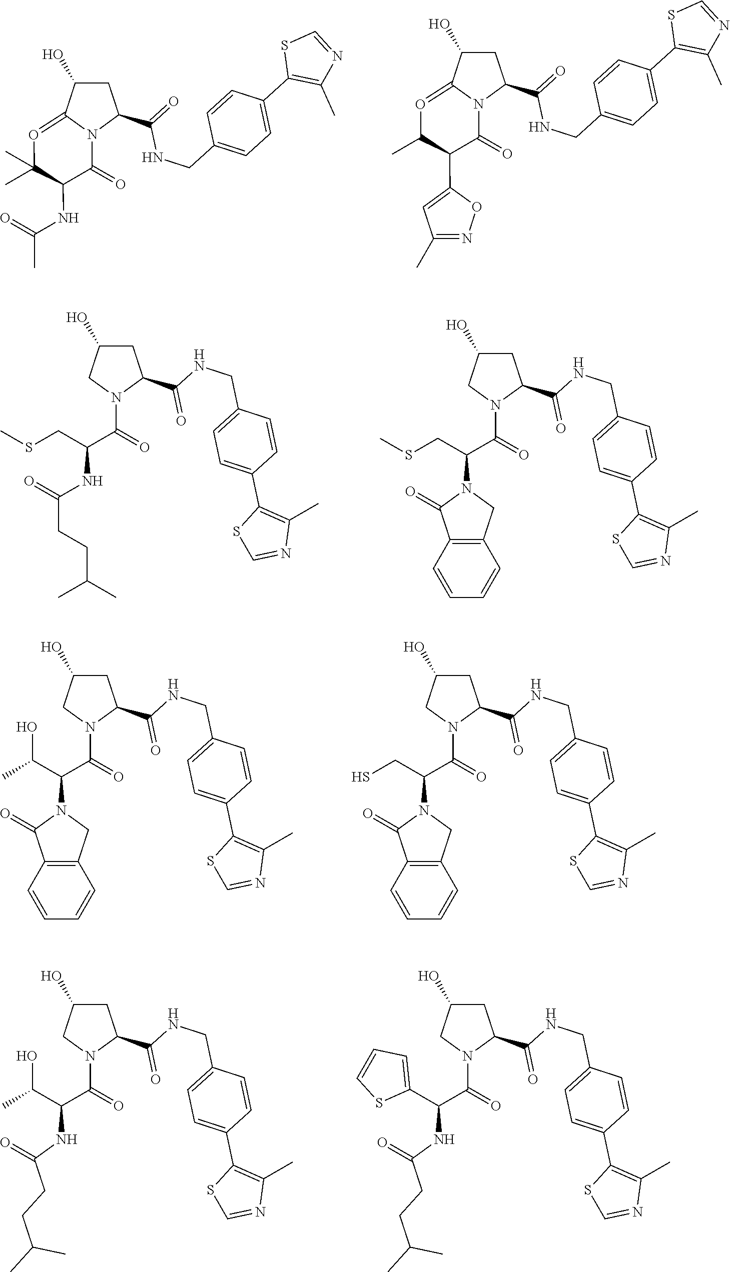

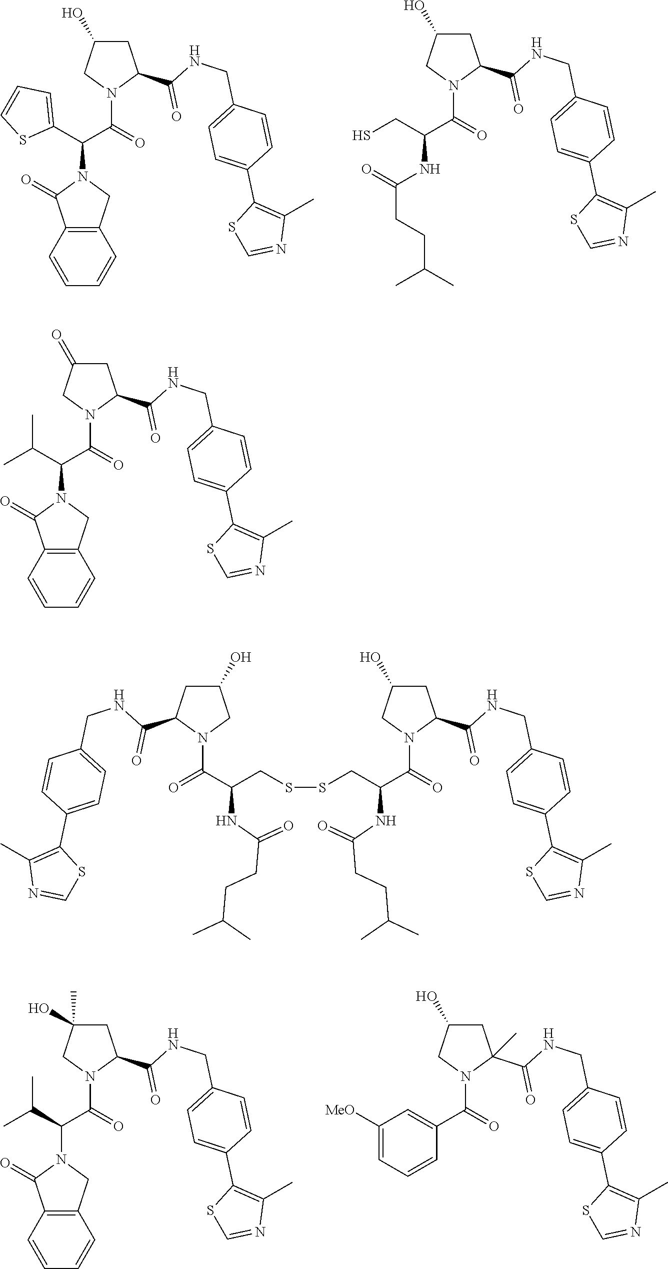

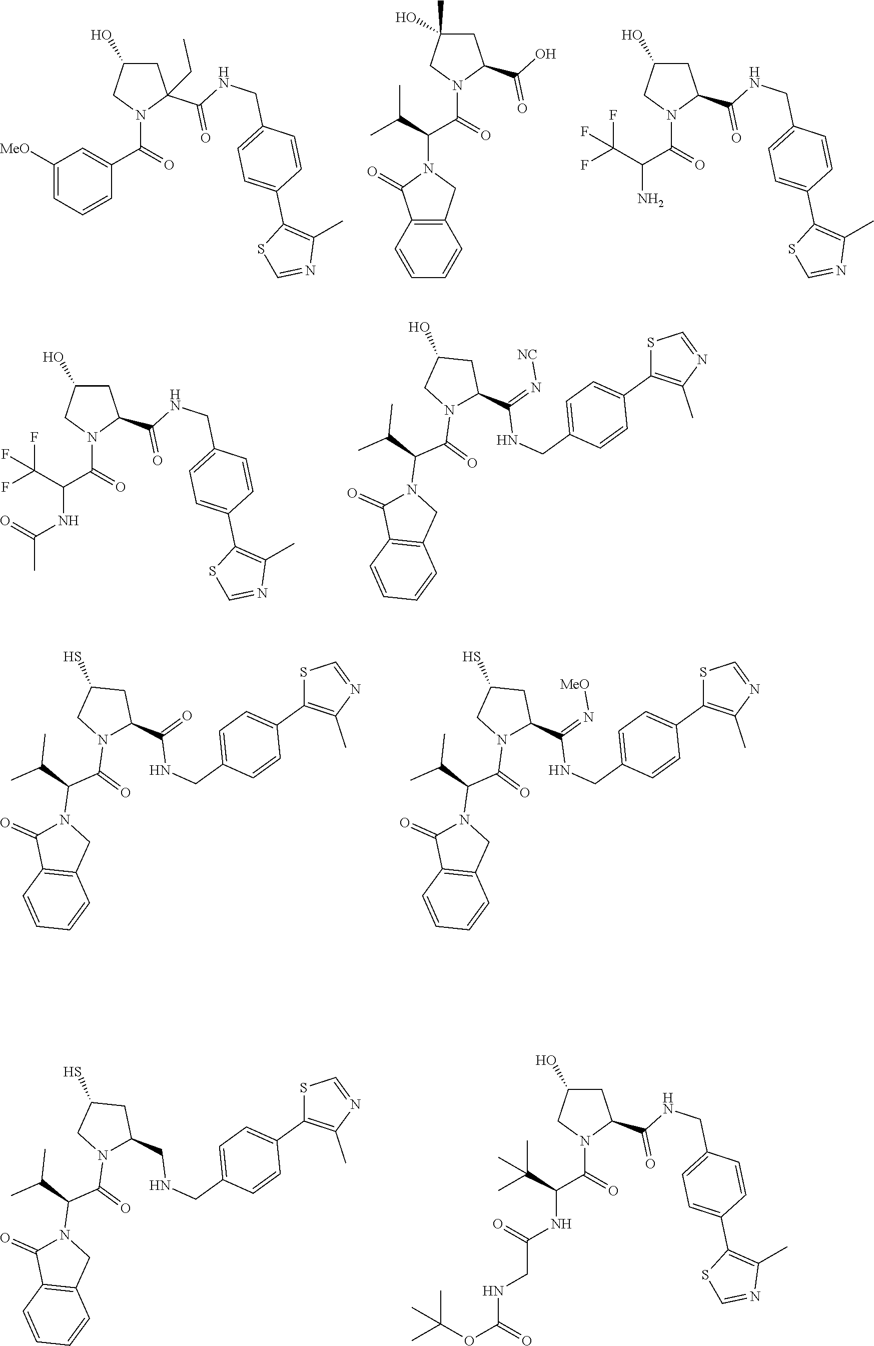

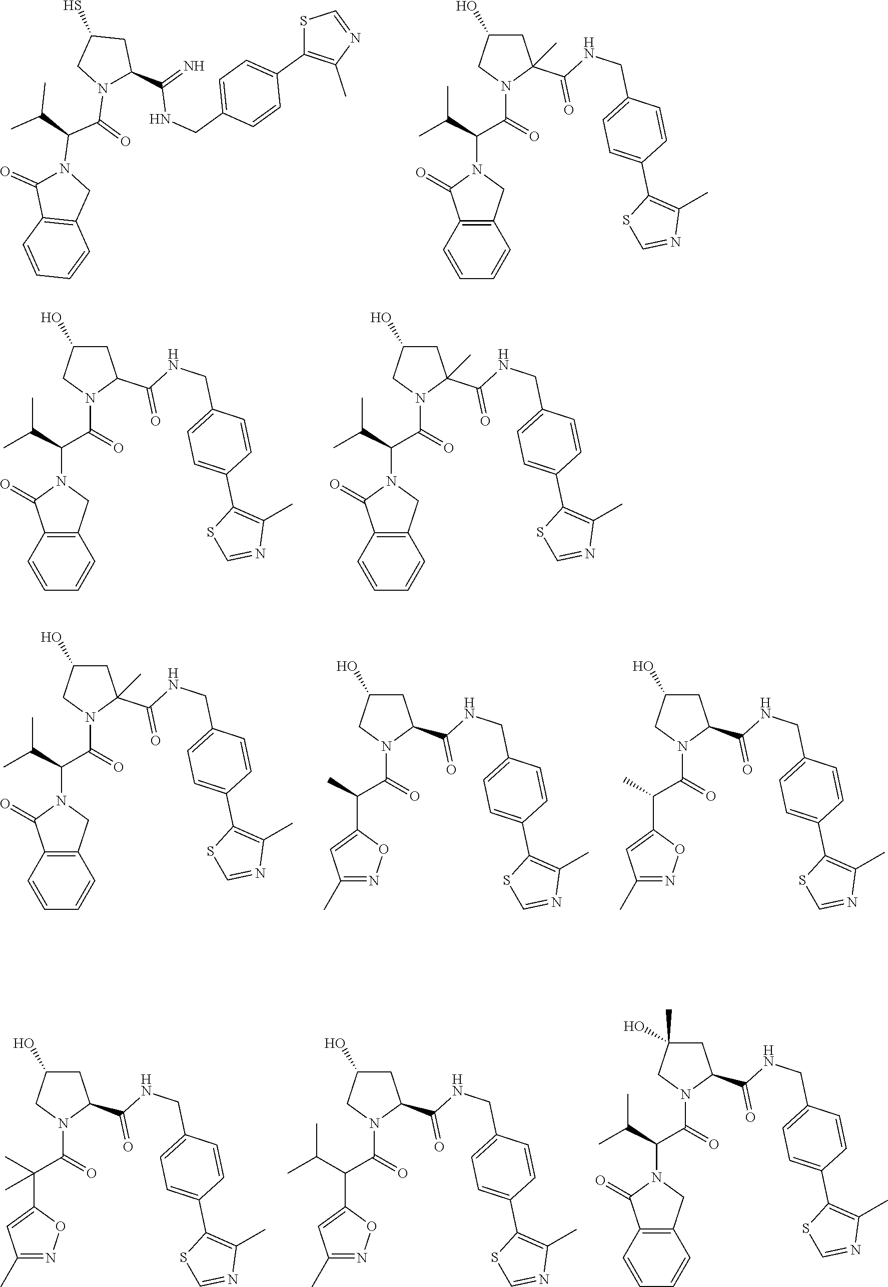

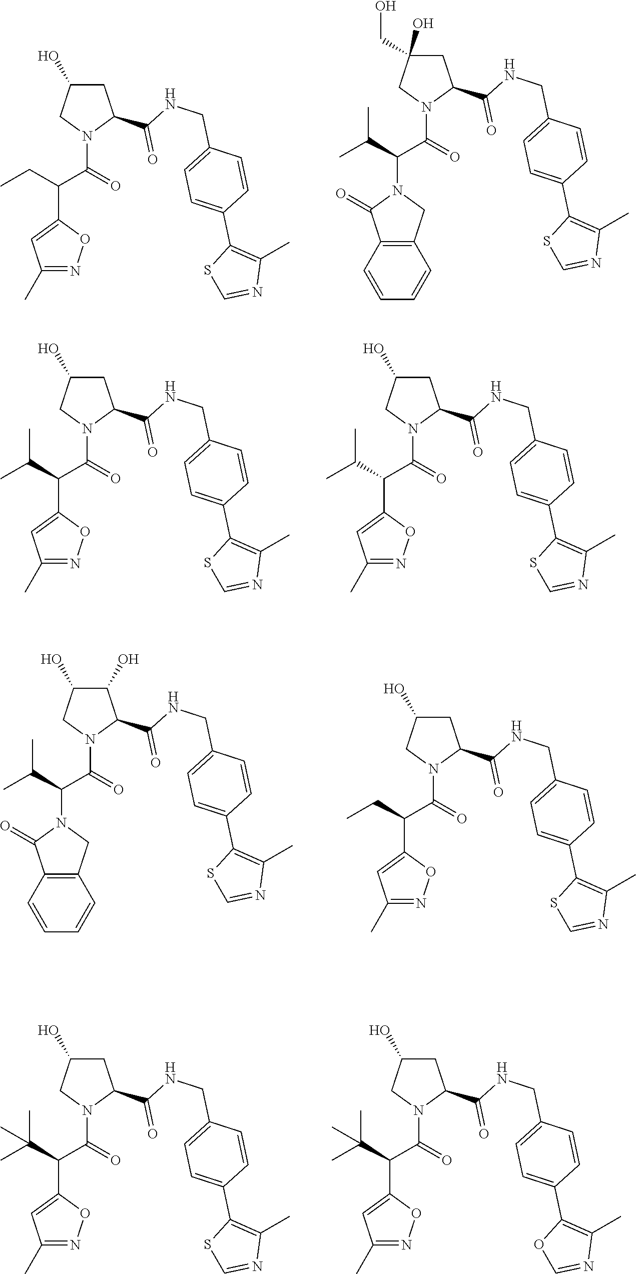

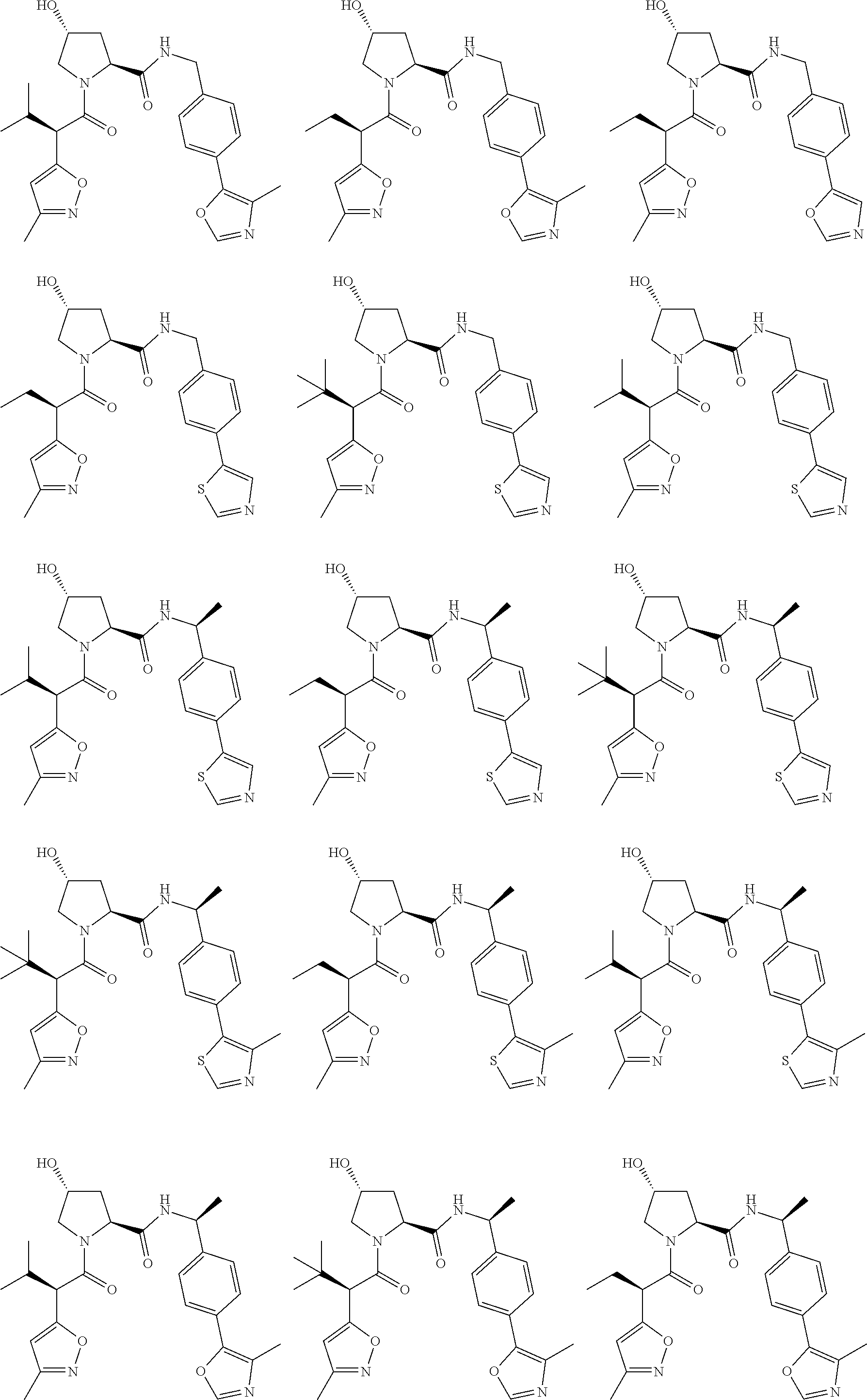

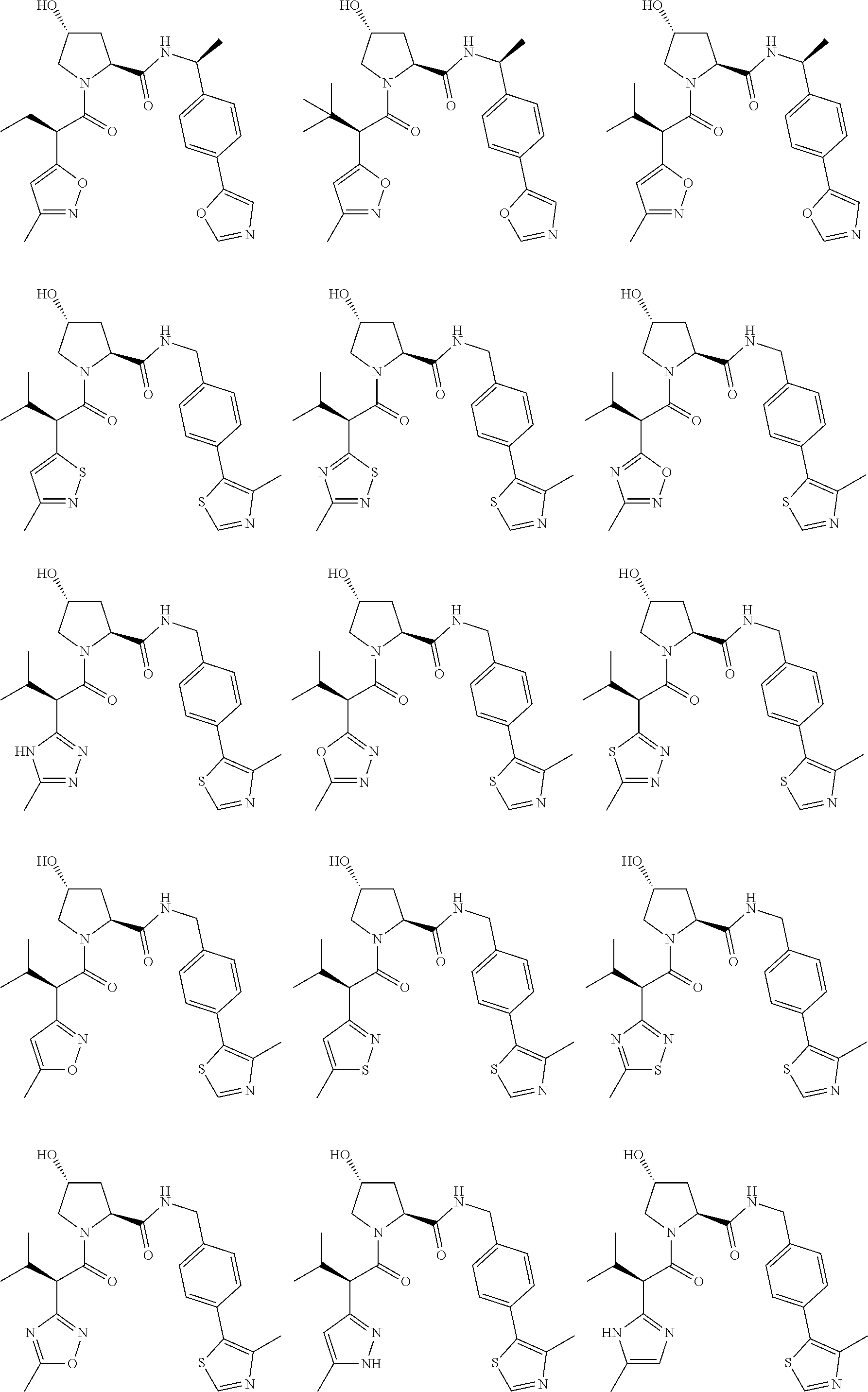

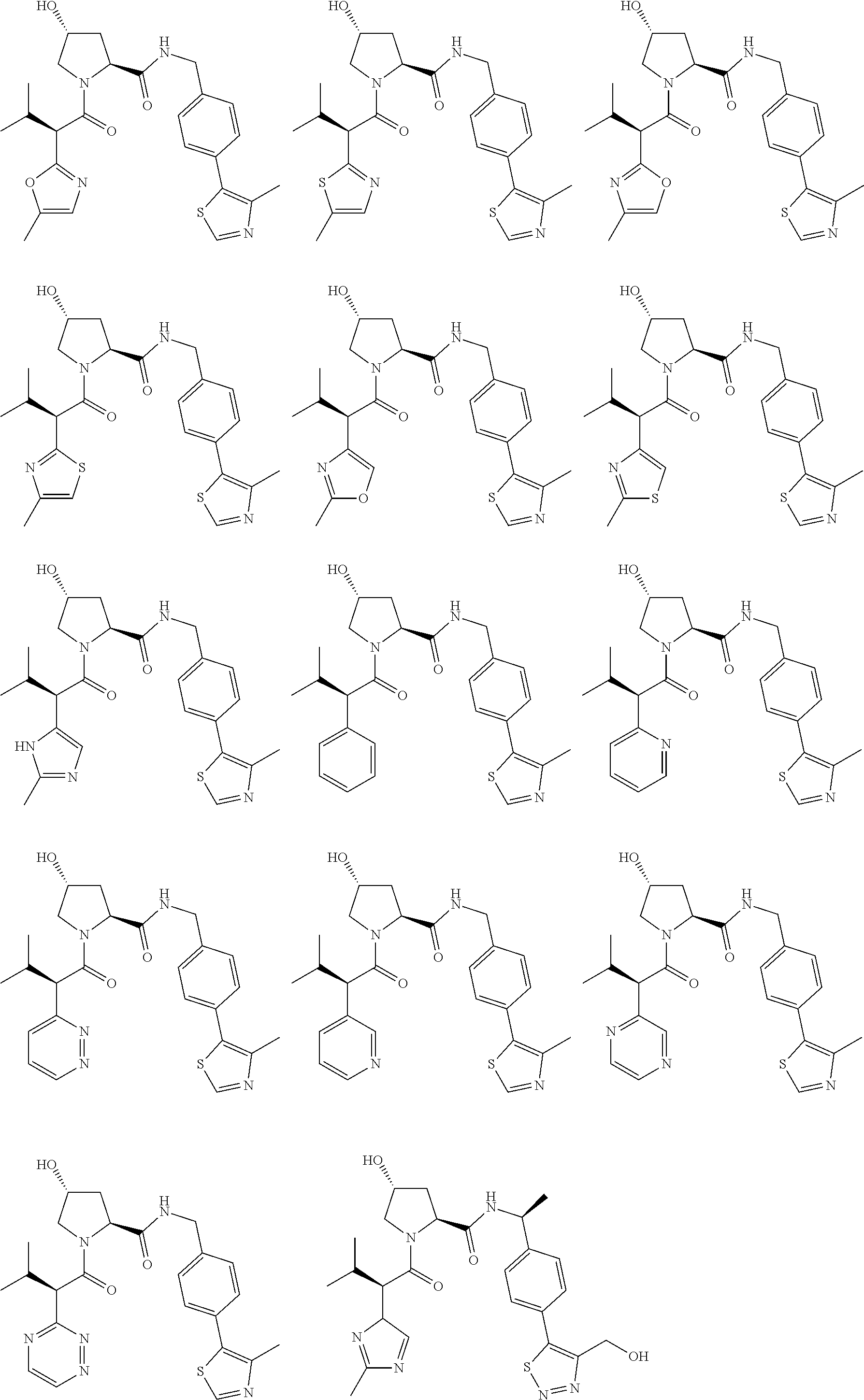

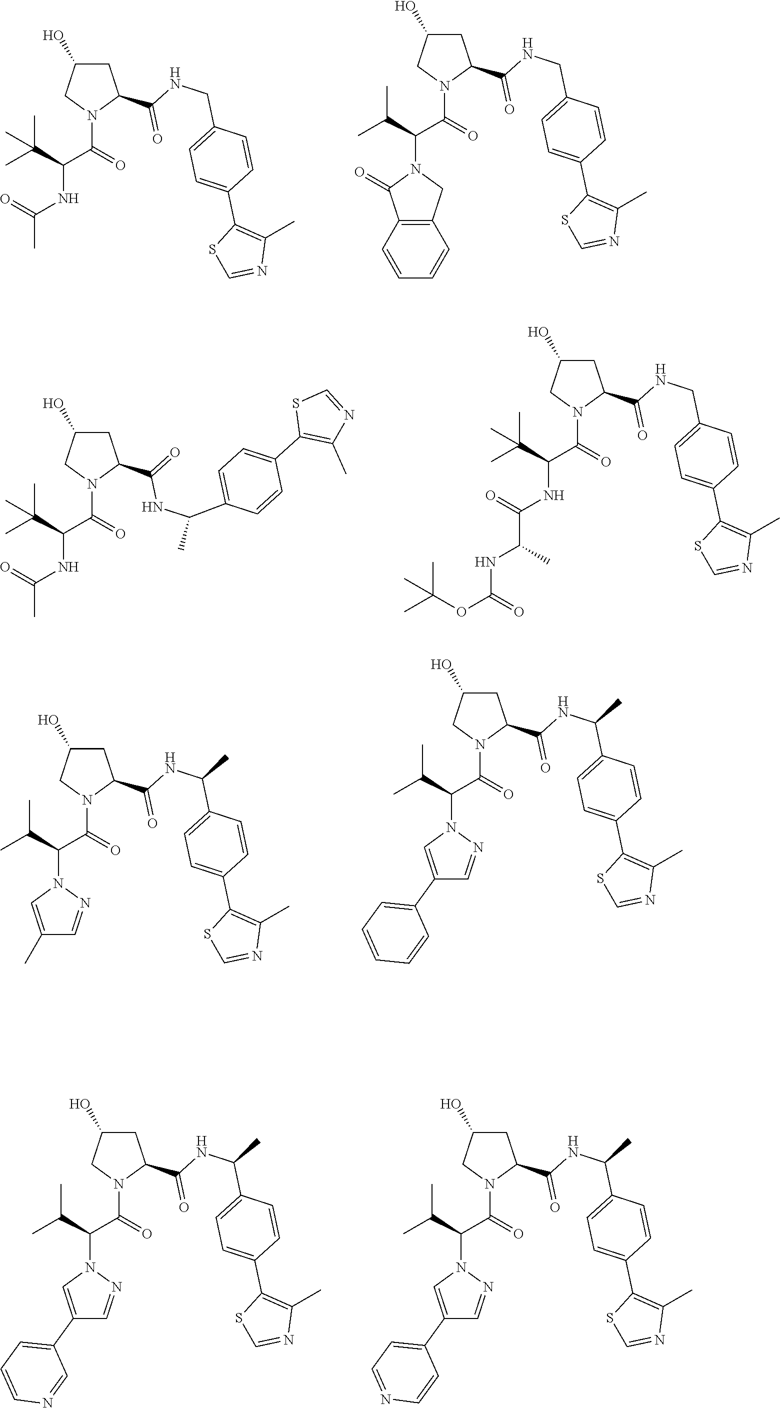

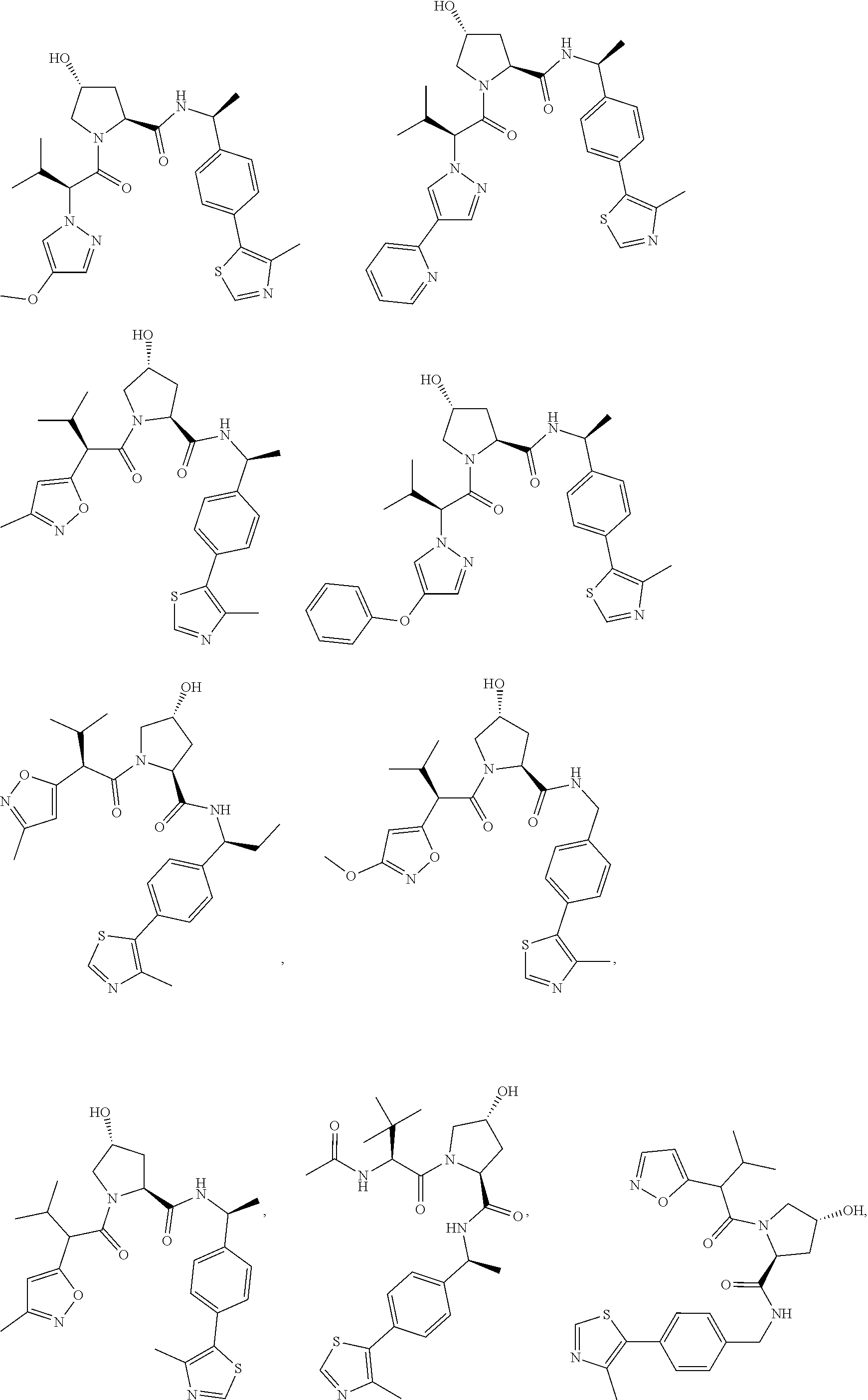

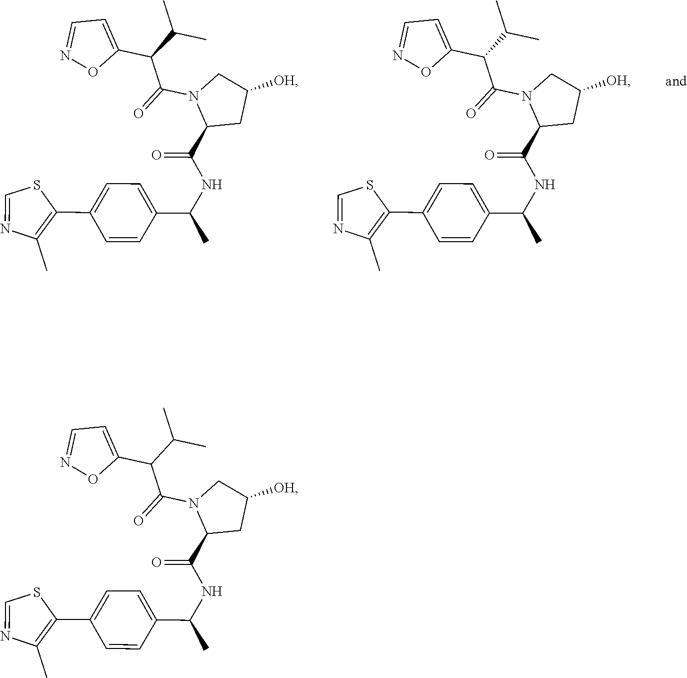 [SMILES: Cc1ncsc1-c1ccc([C@H](C)NC(=O)[C@@H]2C[C@@H](O)CN2C(=O)[C@@H](c2ccno2)C(C)C)cc1 ; Cc1ncsc1-c1ccc([C@H](C)NC(=O)[C@@H]2C[C@@H](O)CN2C(=O)[C@H](c2ccno2)C(C)C)cc1 ; Cc1ncsc1-c1ccc([C@H](C)NC(=O)[C@@H]2C[C@@H](O)CN2C(=O)C(c2ccno2)C(C)C)cc1]

wherein the VLM may be connected to a PTM via a linker, as described herein, at any appropriate location, including, e.g., an aryl, heteroary, phenyl, or phenyl of an indole group, optionally via any appropriate functional group, such as an amine, ester, ether, alkyl, or alkoxy.

### Exemplary Linkers

In certain embodiments, the compounds as described herein include one or more PTMs chemically linked or coupled to one or more ULMs (e.g., at least one of CLM, VLM, MLM, ILM, or a combination thereof) via a chemical linker (L). In certain embodiments, the linker group L is a group comprising one or more covalently connected structural units (e.g., -A₁ . . . (A^{L})_{q}- or -(A^{L})_{q}-), wherein A^{L}₁ is a group coupled to PTM, and (A^{L})_{q} is a group coupled to ULM.

In any aspect or embodiment described herein, the linker (L) to ULM (e.g., VLM, ILM, CLM, or MLM) connection or coupling is a stable L-ULM connection. For example, in any aspect or embodiment described herein, when a linker (L) and a ULM is connected via a heteroatom, any subsequent heteroatom, if present, is separated by at least one single carbon atom (e.g., —CH₂—), such as with an acetal or aminal group. By way of further example, in any aspect or embodiment described herein, when a linker (L) and a ULM is connected via a heteroatom, the heteroatom is not part of a ester.

In any aspect or embodiment described herein, the linker group L is a bond or a chemical linker group represented by the formula -(A^{L})_{q}-, wherein A is a chemical moiety and q is an integer from 1-100 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80), and wherein L is covalently bound to the PTM and the ULM, and provides for sufficient binding of the PTM to the protein target and the ULM to an E3 ubiquitin ligase to result in target protein ubiquitination.

In any aspect or embodiment described herein, the linker group L is -(A^{L})_{q}-, wherein:
(A^{L})_{q} is a group which is connected to at least one of a ULM (such as a CLM or a VLM), PTM moiety, or a combination thereof;
q of the linker is an integer greater than or equal to 1;
each A^{L} is independently selected from the group consisting of, a bond, CR^{L1}R^{L2}, O, S, SO, SO₂, NR^{L3}, SO₂NR^{L3}, SONR^{L3}, CONR^{L3}, NR^{L3}CONR^{L4}, NR^{L3}SO₂NR^{L4}, CO, CR^{L1}═CR^{L2}, C≡C, SiR^{L1}R^{L2}, P(O)R^{L1}, P(O)OR^{L1}, NR^{L3}C(═NCN)NR^{L4}, NR^{U}C(═NCN), NR^{L3}C(═CNO₂)NR^{L4}, C₃₋₁₁cycloalkyl optionally substituted with 0-6 R^{L1} and/or R^{L2} groups, C₅₋₁₃ spirocycloalkyl optionally substituted with 0-9 R^{L1} and/or R^{L2} groups, C₃₋₁₁heterocyclyl optionally substituted with 0-6 R^{L1} and/or R^{L2} groups, C₅₋₁₃ spiroheterocycloalkyl optionally substituted with 0-8 R^{L1} and/or R^{L2} groups, aryl optionally substituted with 0-6 R^{L1} and/or R^{L2} groups, heteroaryl optionally substituted with 0-6 R^{L1} and/or R^{L2} groups, where R^{L1} or R^{L2}, each independently are optionally linked to other groups to form cycloalkyl and/or heterocyclyl moiety, optionally substituted with 0-4 R^{L5} groups; and
R^{L1}, R^{L2}, R^{L3}, R^{L4} and R^{L5} are, each independently, H, halo, C₁₋₈alkyl, OC₁₋₈alkyl, SC₁₋₈alkyl, NHC₁₋₈alkyl, N(C₁₋₈alkyl)₂, C₃₋₁₁cycloalkyl, aryl, heteroaryl, C₃₋₁₁heterocyclyl, OC₃₋₈cycloalkyl, SC₃₋₈cycloalkyl, NHC₃₋₈cycloalkyl, N(C₃₋₈cycloalkyl)₂, N(C₃₋₈cycloalkyl)(C₁₋₈alkyl), OH, NH₂, SH, SO₂C₁₋₈alkyl, P(O)(OC₁₋₈alkyl)(C₁₋₈alkyl), P(O)(OC₁₋₈alkyl)₂, CC—C₁₋₈alkyl, CCH, CH═CH(C₁₋₈alkyl), C(C₁₋₈alkyl)═CH(C₁₋₈alkyl), C(C₁₋₈alkyl)═C(C₁₋₈alkyl)₂, Si(OH)₃, Si(C₁₋₈alkyl)₃, Si(OH)(C₁₋₈alkyl)₂, COC₁₋₈alkyl, CO₂H, halogen, CN, CF₃, CHF₂, CH₂F, NO₂, SF₅, SO₂NHC₁₋₈alkyl, SO₂N(C₁₋₈alkyl)₂, SONHC₁₋₈alkyl, SON(C₁₋₈alkyl)₂, CONHC₁₋₈alkyl, CON(C₁₋₈alkyl)₂, N(C₁₋₈alkyl)CONH(C₁₋₈alkyl), N(C₁₋₈alkyl)CON(C₁₋₈alkyl)₂, NHCONH(C₁₋₈alkyl), NHCON(C₁₋₈alkyl)₂, NHCONH₂, N(C₁₋₈alkyl)SO₂NH(C₁₋₈alkyl), N(C₁₋₈alkyl) SO₂N(C₁₋₈alkyl)₂, NHSO₂NH(C₁₋₈alkyl), NHSO₂N(C₁₋₈alkyl)₂, NHSO₂NH₂.

In certain embodiments, q of the linker is an integer greater than or equal to 0. In certain embodiments, q is an integer greater than or equal to 1.

In certain embodiments, e.g., where q of the linker is greater than 2, (A^{L})_{q} is a group which A^{L}₁ and (A^{L})_{q} wherein the units A^{L} are couple a PTM to a ULML).

In certain embodiments, e.g., where q of the linker is 2, (A^{L})_{q} is a group which is connected to A^{L}₁ and to a ULM.

In certain embodiments, e.g., where q of the linker is 1, the structure of the linker group L is -A^{L}₁-, and A^{L}₁ is a group which is connected to a ULM moiety and a PTM moiety.

In certain embodiments, the unit A^{L} of linker (L) comprises a group represented by a general structure selected from the group consisting of:
—NR(CH₂)ₙ-(lower alkyl)-, —NR(CH₂)ₙ-(lower alkoxyl)-, —NR(CH₂)ₙ-(lower alkoxyl)-OCH₂—, —NR(CH₂)ₙ-(lower alkoxyl)-(lower alkyl)-OCH₂—, —NR(CH₂)ₙ-(cycloalkyl)-(lower alkyl)-OCH₂-, —NR(CH₂)ₙ-(hetero cycloalkyl)-, —NR(CH₂CH₂O)ₙ-(lower alkyl)-O—CH₂—, —NR(CH₂CH₂O)ₙ-(hetero cycloalkyl)-O—CH₂—, —NR(CH₂CH₂O)ₙ-Aryl-O—CH₂—, NR(CH₂CH₂O)ₙ-(hetero aryl)-O—CH₂—, —NR(CH₂CH₂O)ₙ-(cyclo alkyl)-O-(hetero aryl)-O—CH₂—, —NR(CH₂CH₂O)ₙ-(cyclo alkyl)-O-Aryl-O—CH₂—, —NR(CH₂CH₂O)ₙ-(lower alkyl)-NH-Aryl-O—CH₂—, —NR(CH₂CH₂O)ₙ-(lower alkyl)-O-Aryl-CH₂, —NR(CH₂CH₂O)ₙ-cycloalkyl-O-Aryl-, —NR(CH₂CH₂O)ₙ-cycloalkyl-O-(heteroaryl)l-, —NR(CH₂CH₂)ₙ-(cycloalkyl)-O-(heterocycle)-CH₂, —NR(CH₂CH₂)ₙ-(heterocycle)-(heterocycle)-CH₂, N(R1R2)-(heterocycle)-CH₂; where
n of the linker can be 0 to 10;
R of the linker can be H, lower alkyl;
R1 and R2 of the linker can form a ring with the connecting N.

In certain embodiments, the unit A^{L} of linker (L) comprises a group represented by a general structure selected from the group consisting of:
—N(R)—(CH₂)ₘ—O(CH₂)ₙ—O(CH₂)ₒ—O(CH₂)ₚ—O(CH₂)_{q}—O(CH₂)ᵣ—OCH₂—,
—O—(CH₂)ₘ—O(CH₂)ₙ—O(CH₂)ₒ—O(CH₂)ₚ—O(CH₂)_{q}—O(CH₂)ᵣ—OCH₂—,
—O—(CH₂)ₘ—O(CH₂)ₙ—O(CH₂)ₒ—O(CH₂)ₚ—O(CH₂)_{q}—O(CH₂)ᵣ—O—;
—N(R)—(CH₂)ₘ—O(CH₂)ₙ—O(CH₂)ₒ—O(CH₂)ₚ—O(CH₂)_{q}—O(CH₂)ᵣ—O—;
—(CH₂)ₘ—O(CH₂)ₙ—O(CH₂)ₒ—O(CH₂)ₚ—O(CH₂)_{q}—O(CH₂)ᵣ—O—;
—(CH₂)ₘ—O(CH₂)ₙ—O(CH₂)ₒ—O(CH₂)ₚ—O(CH₂)_{q}—O(CH₂)ᵣ—OCH₂—;

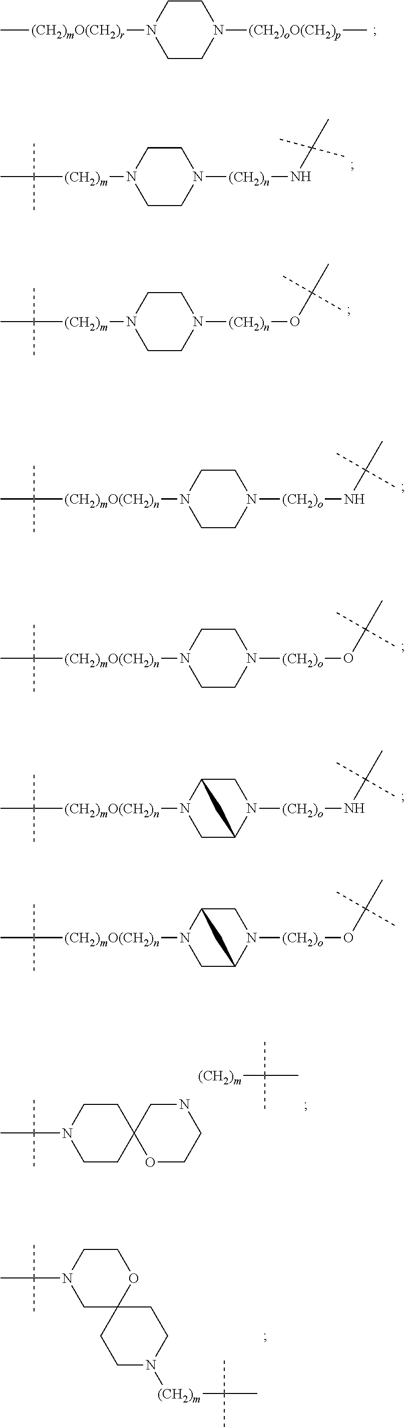

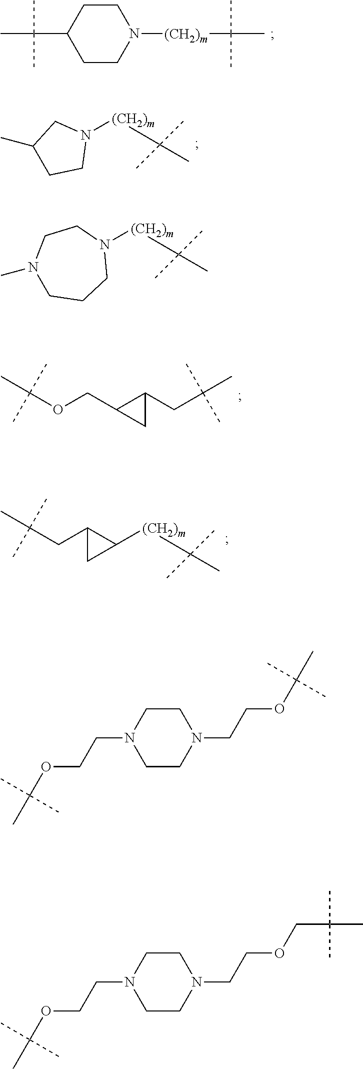

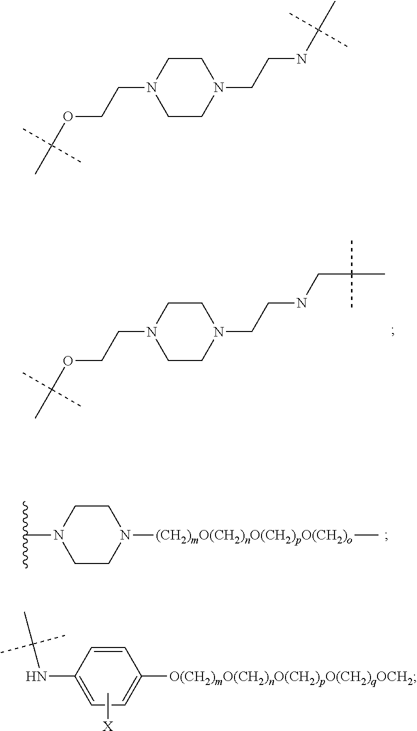

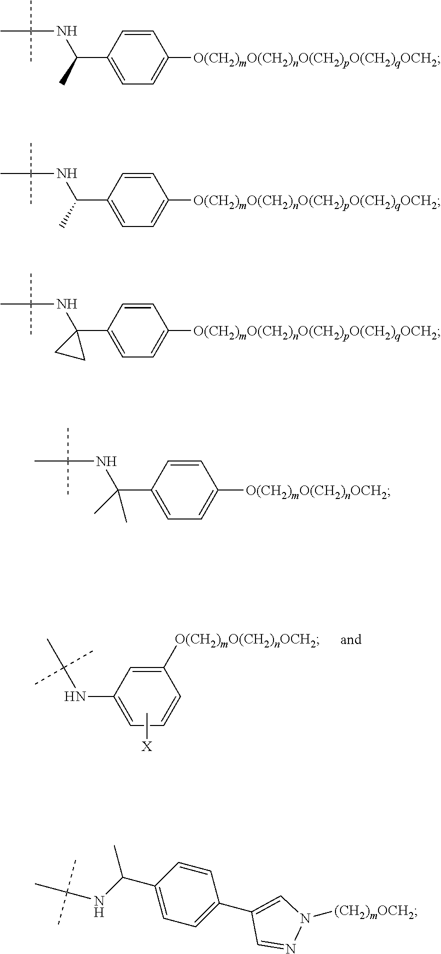

wherein

m, n, o, p, q, and r of the linker are independently 0, 1, 2, 3, 4, 5, 6, 7, 8, 9, 10, 11, 12, 13, 14, 15, 16, 17, 18, 19, 20;

when the number is zero, there is no N—O or O—O bond

R of the linker is H, methyl and ethyl;

X of the linker is H and F

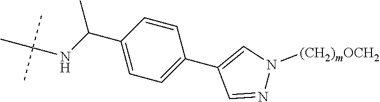

where m of the linker can be 2, 3, 4, 5

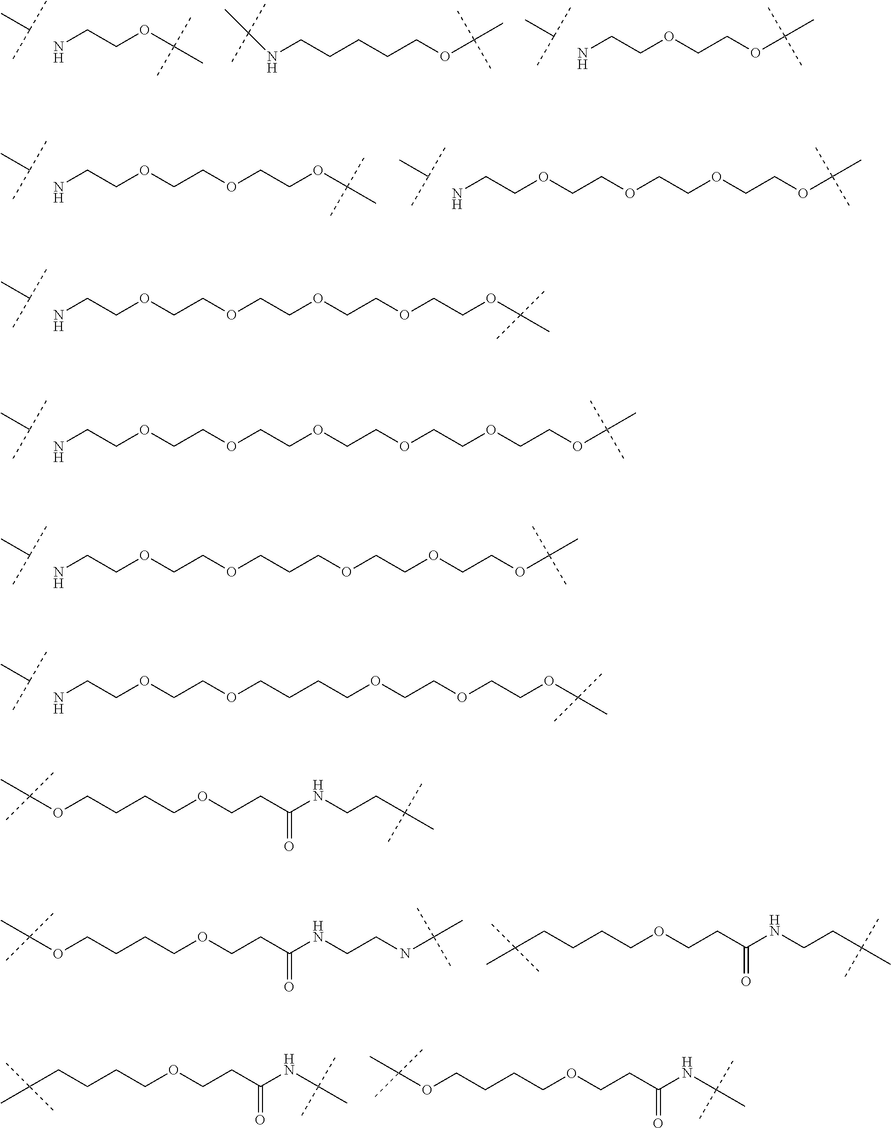

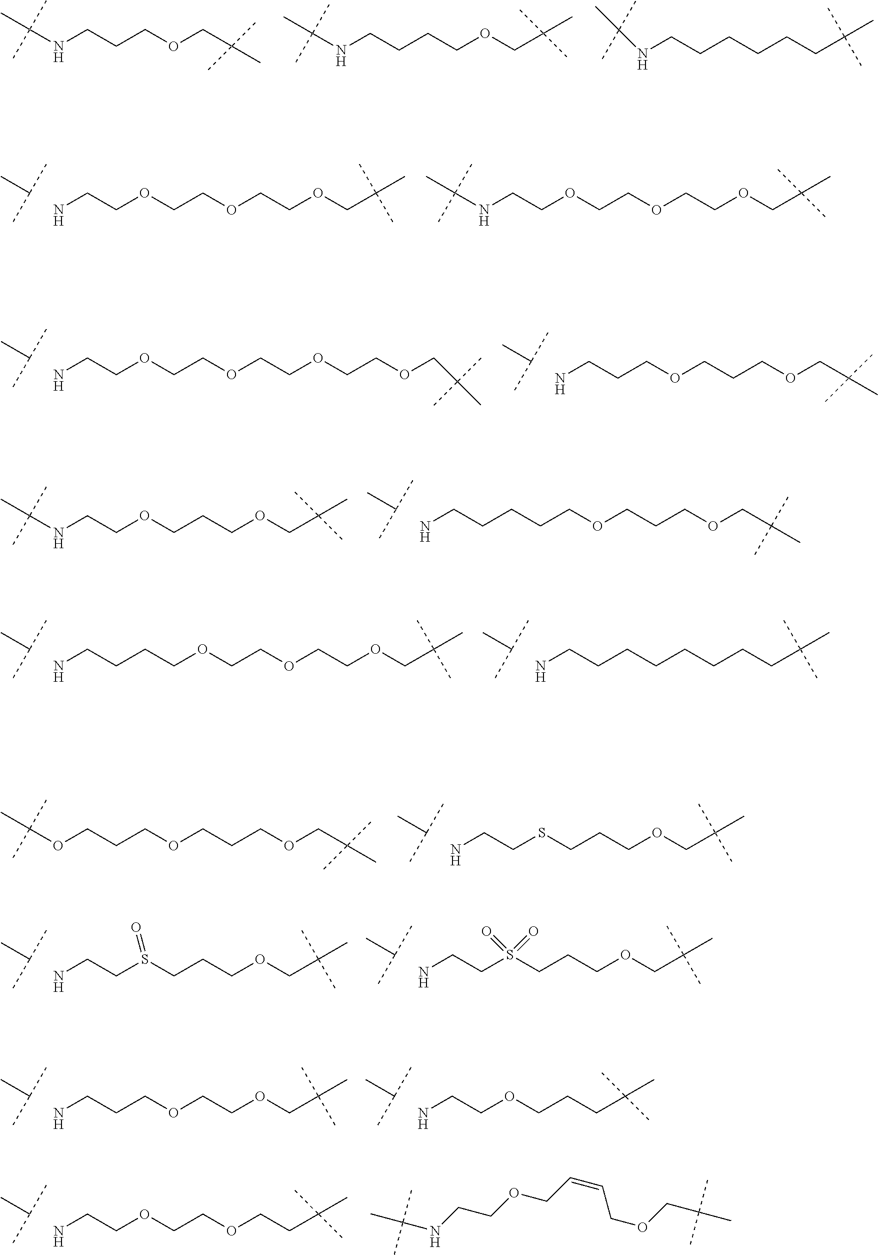

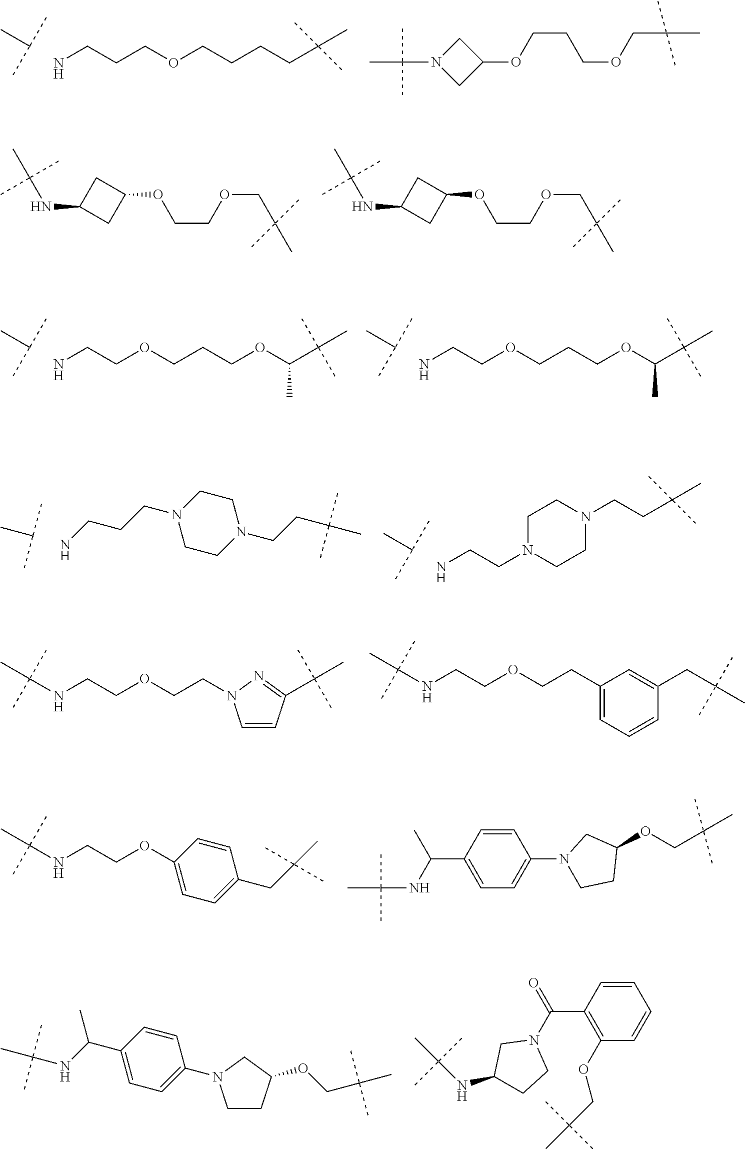

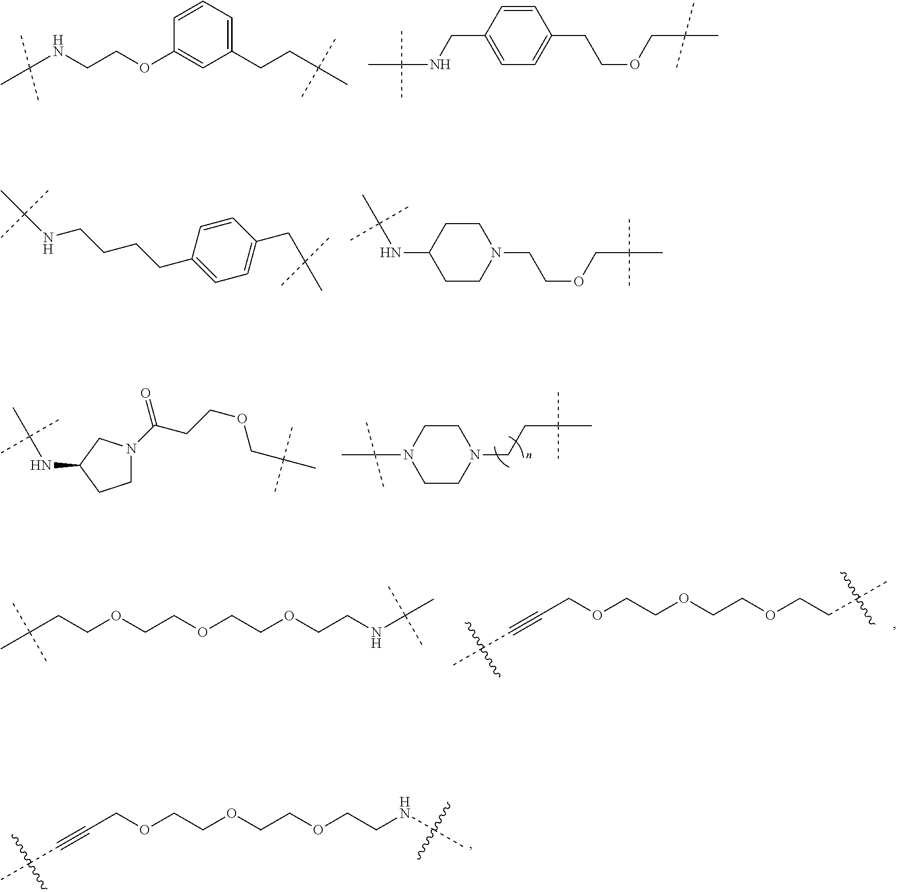

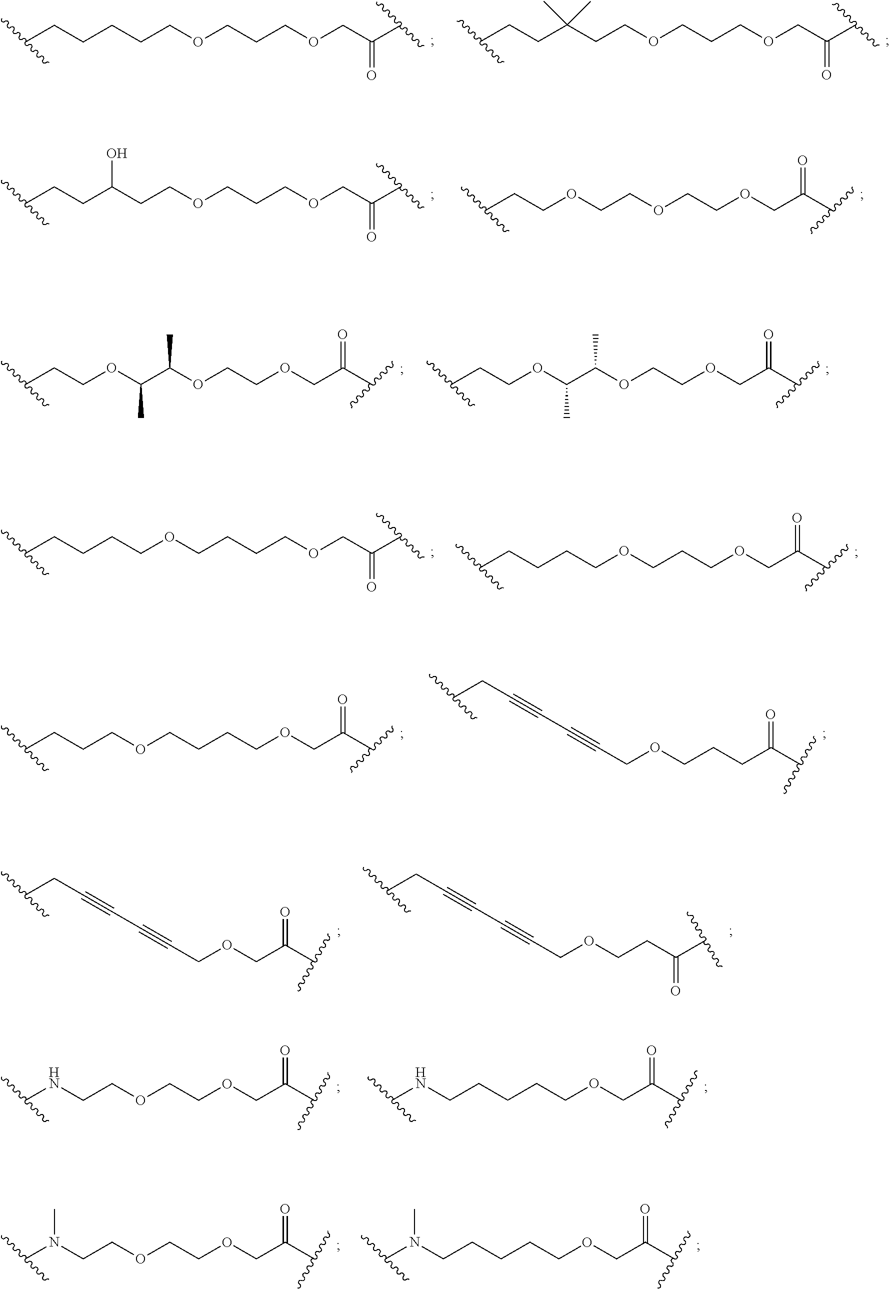

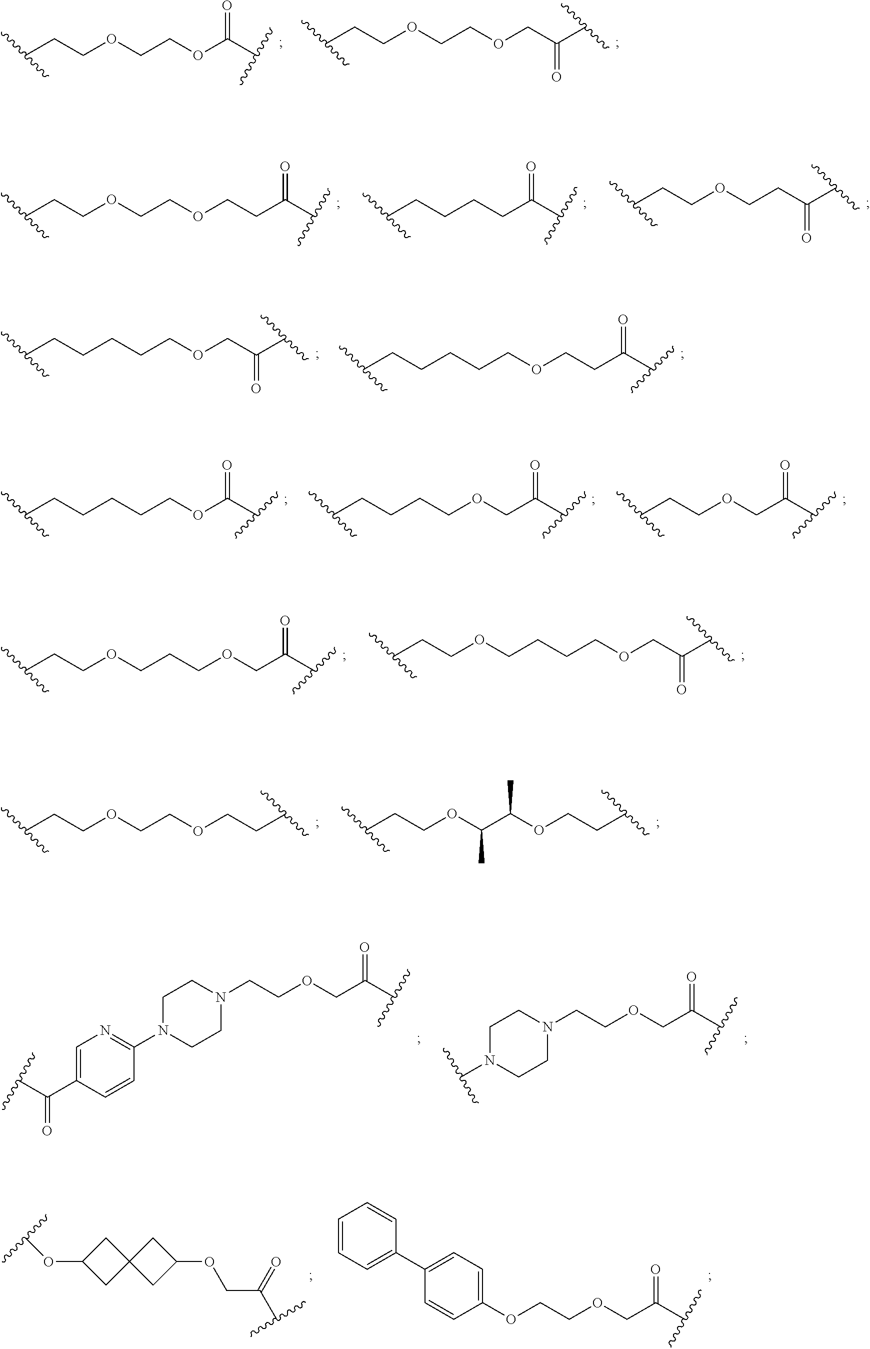

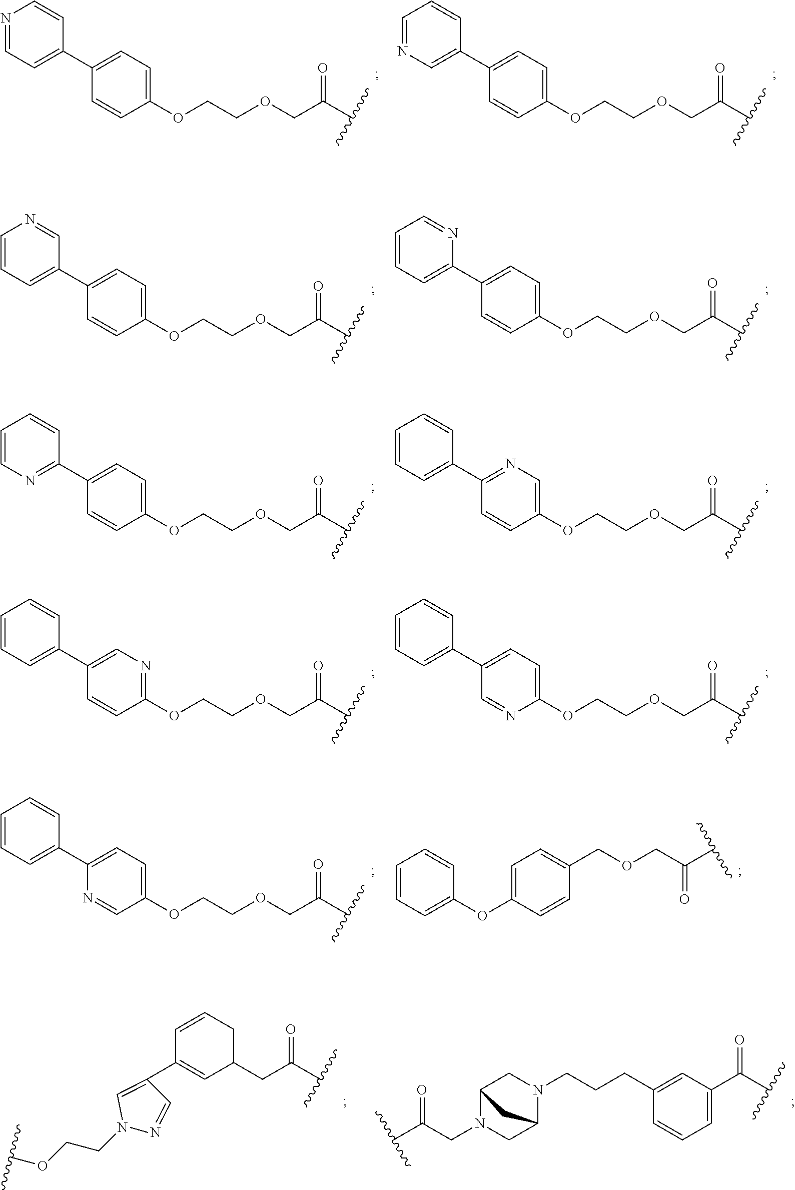

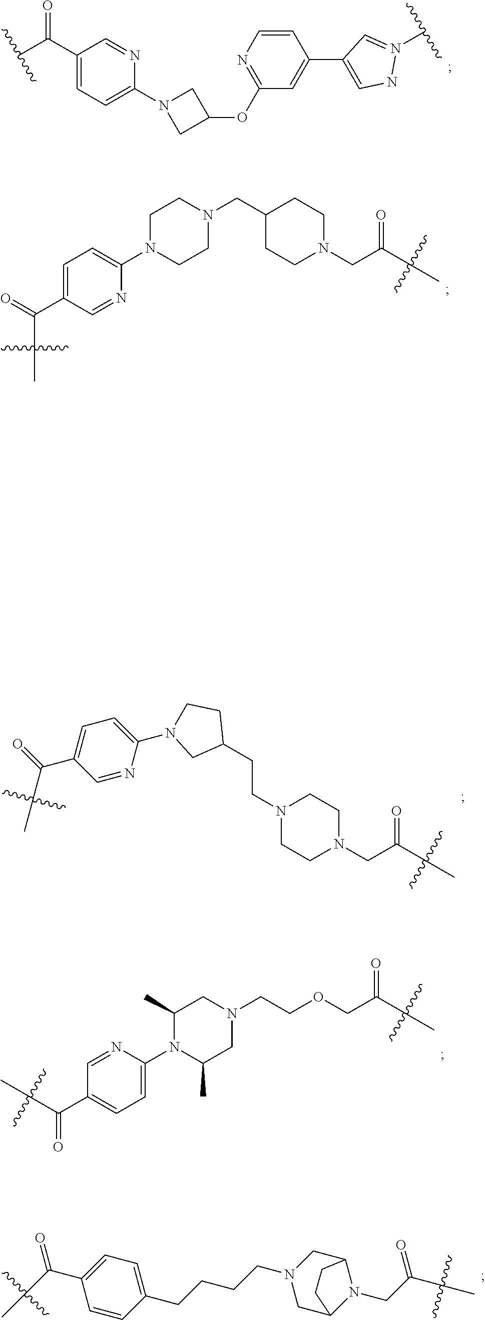

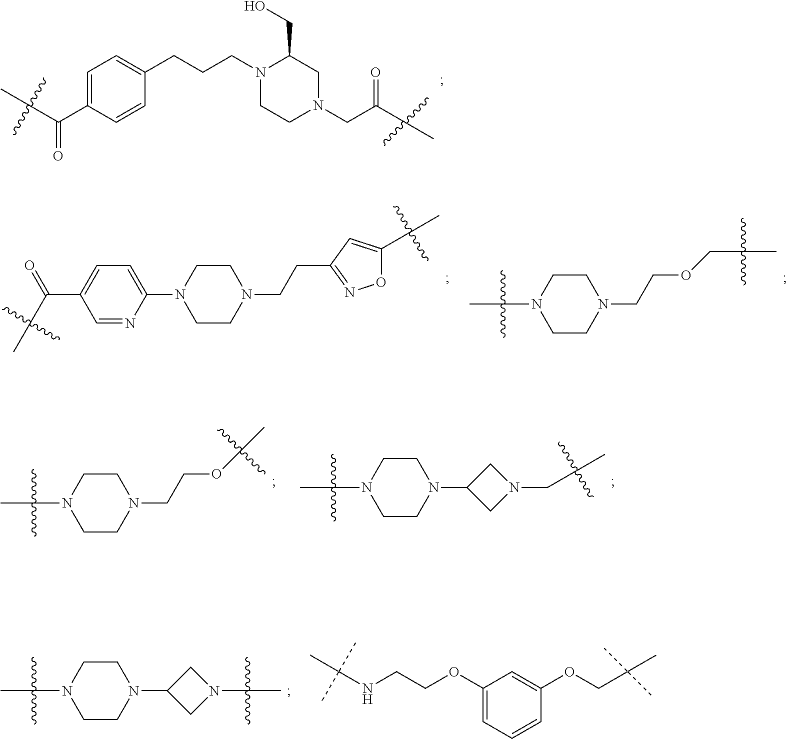

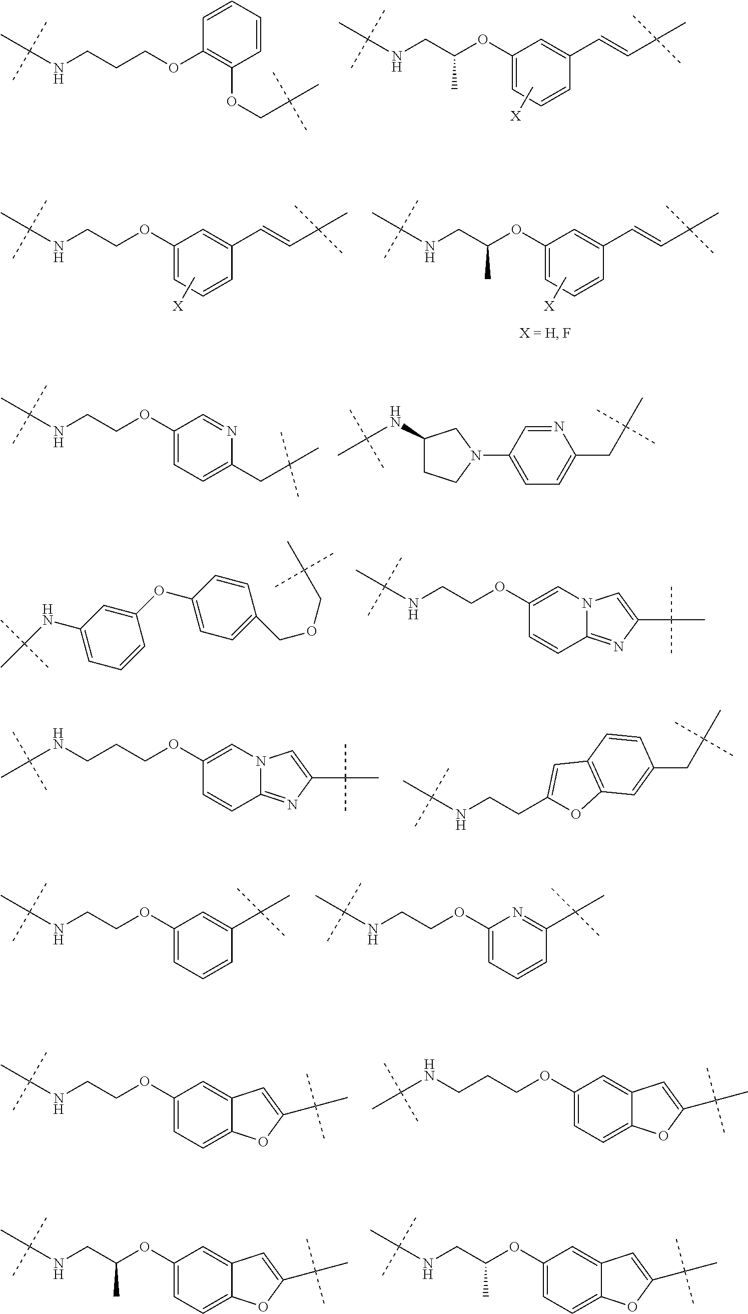

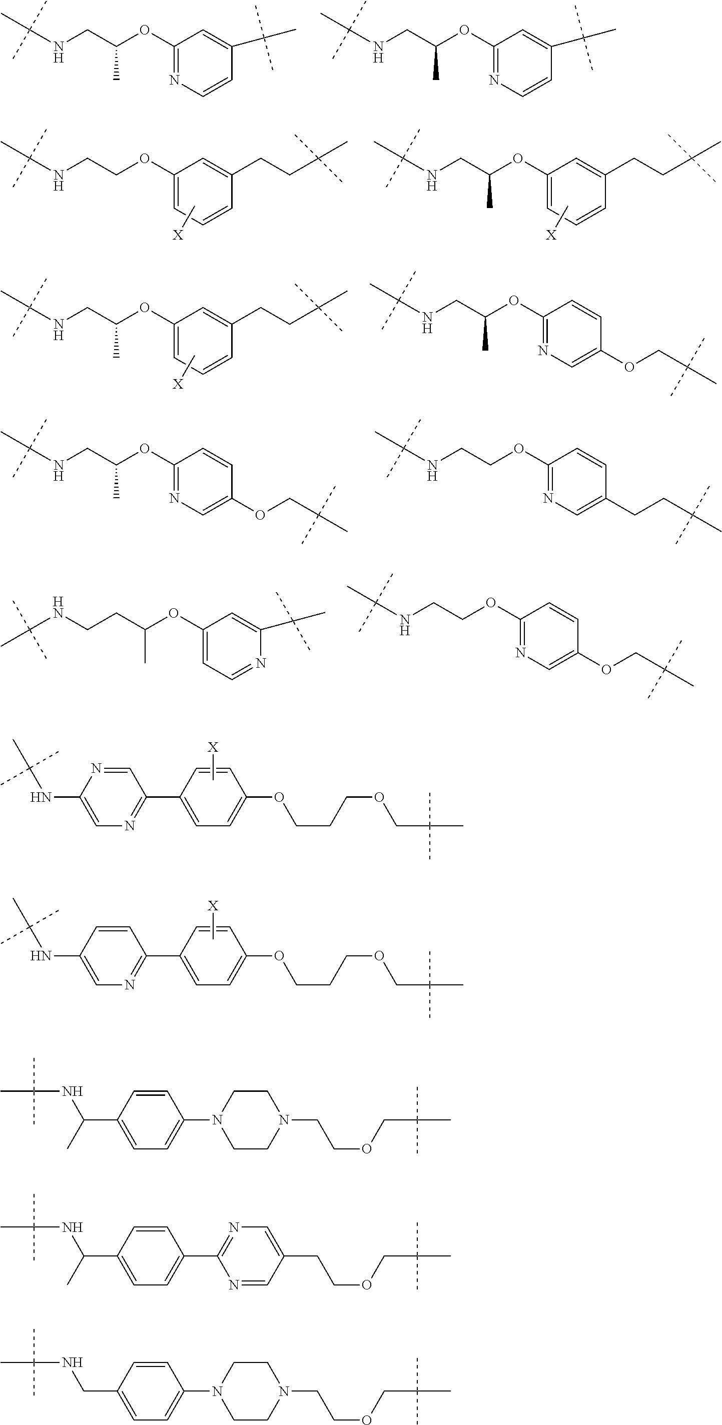

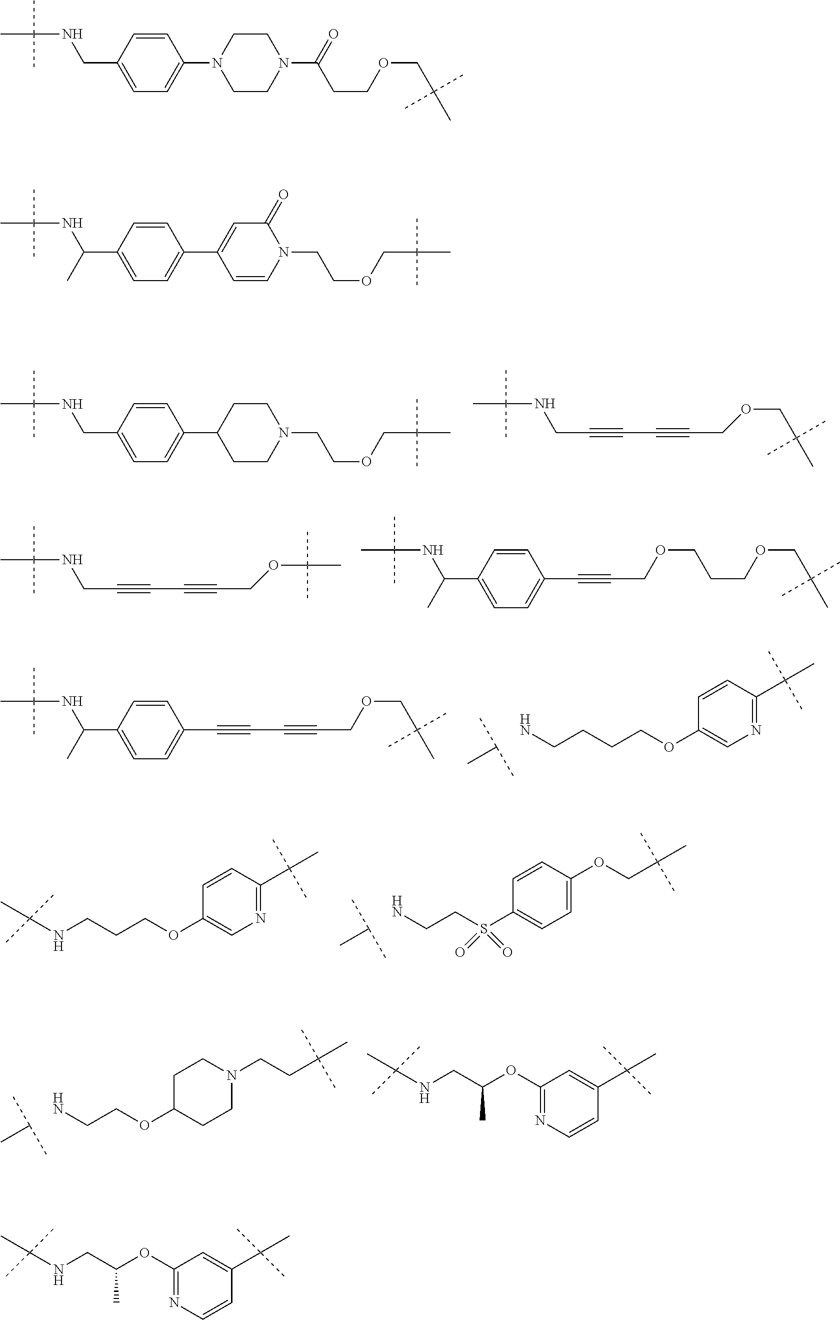

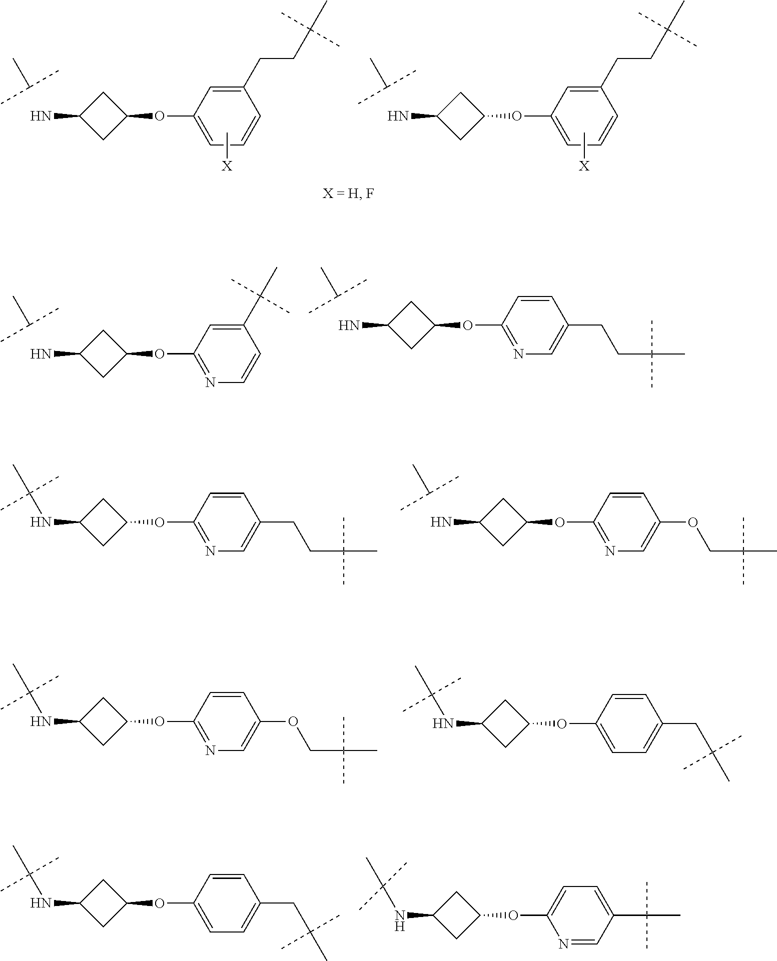

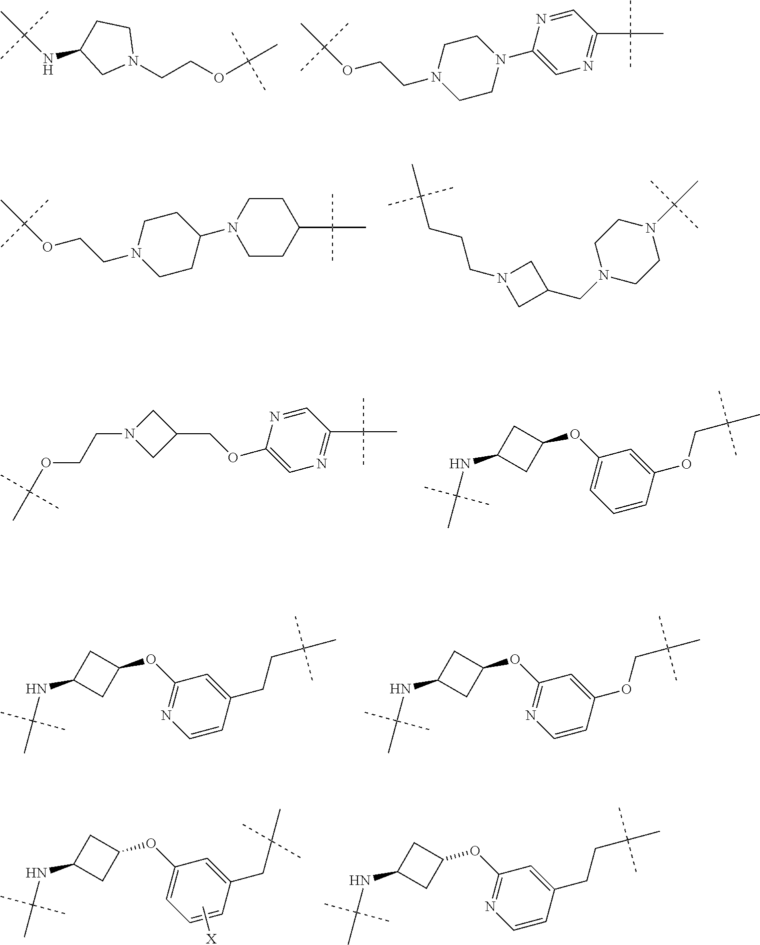

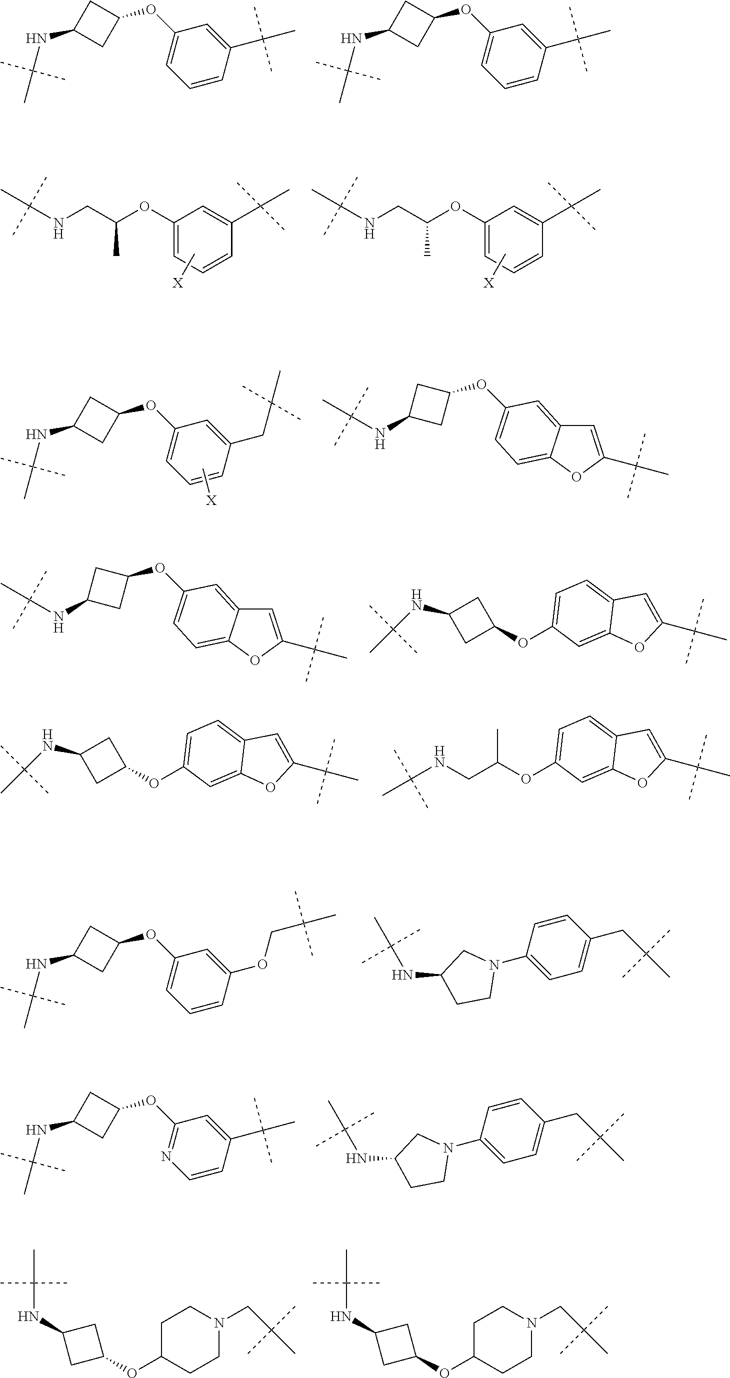

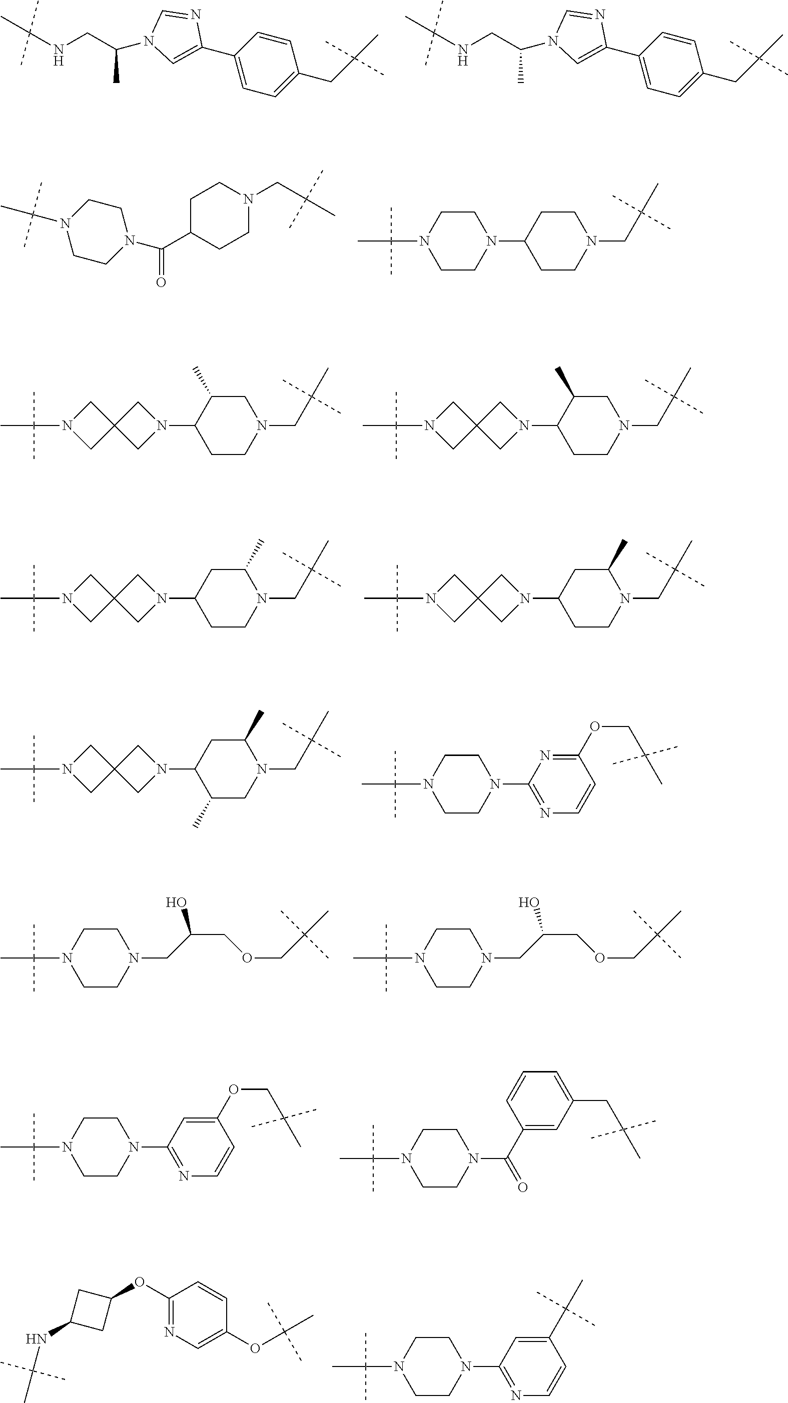

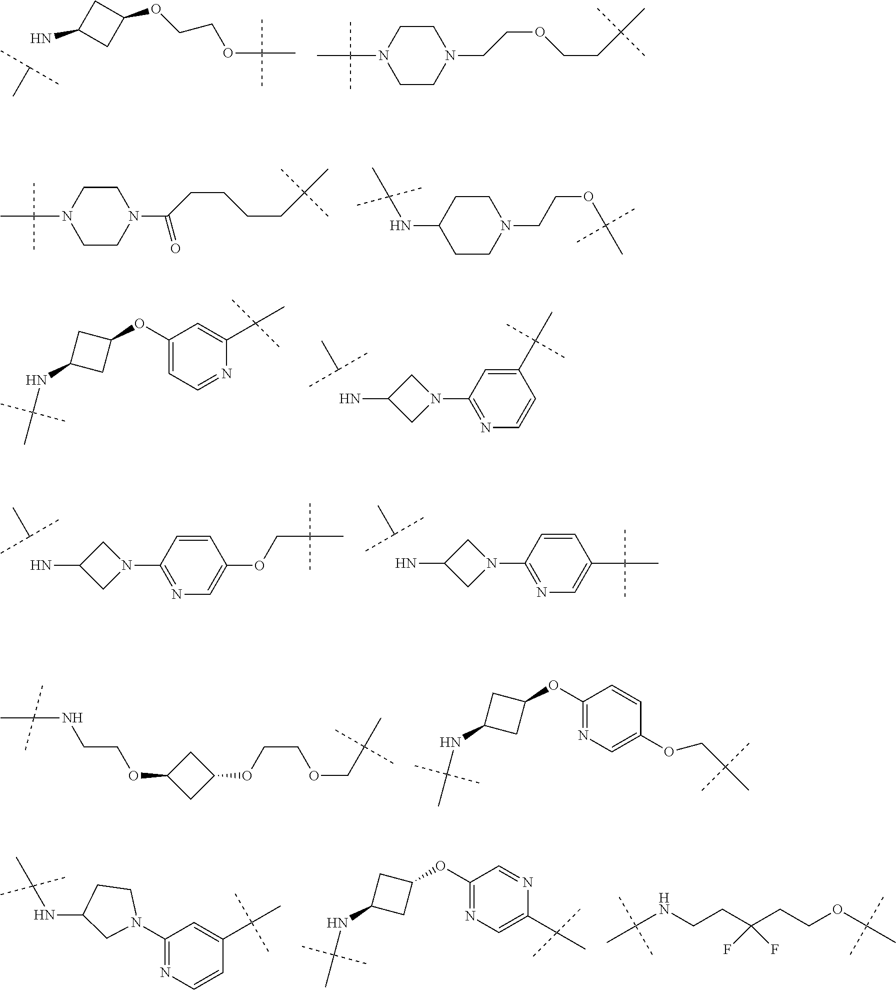

where each n and m of the linker can independently be 0, 1, 2, 3, 4, 5, 6.

In any aspect or embodiment described herein, the unit A^{L} of linker (L) is selected from the group consisting of:

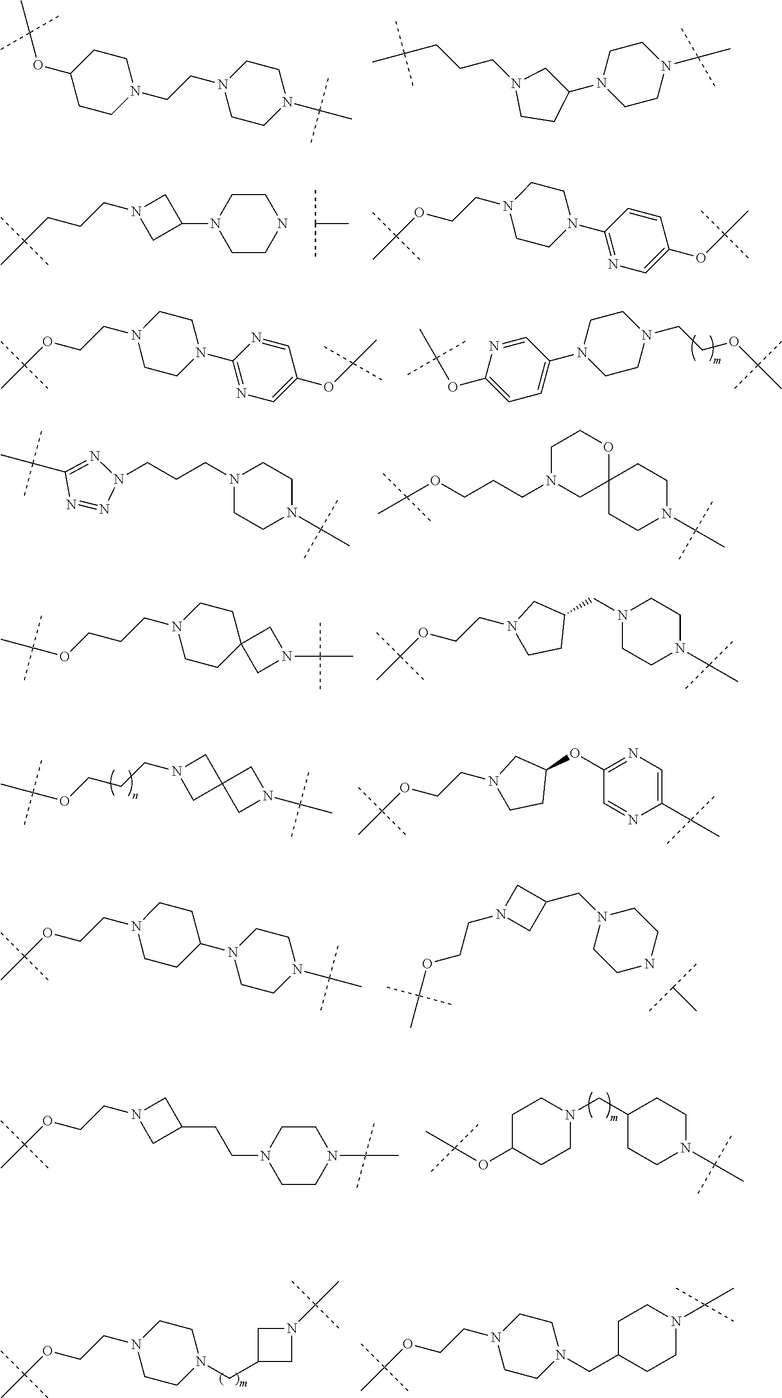

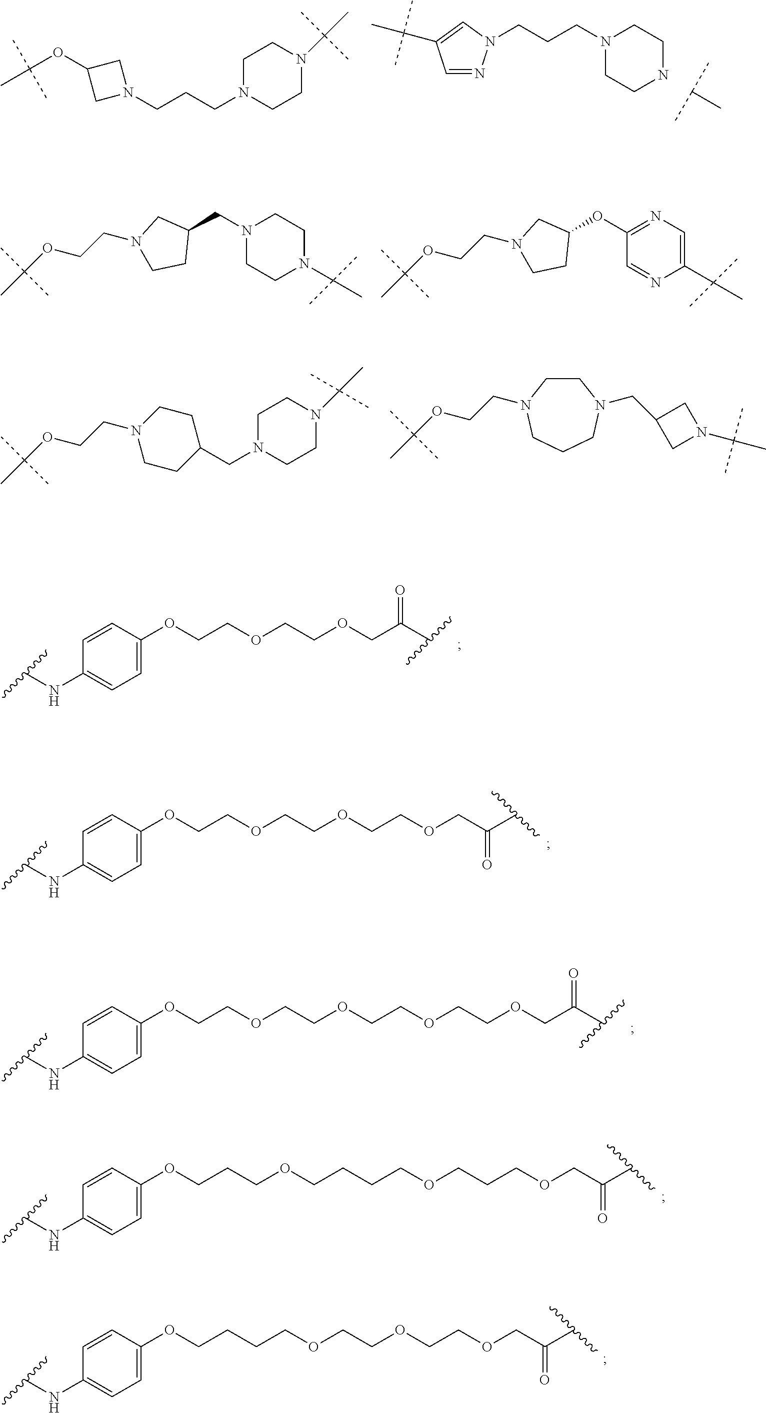

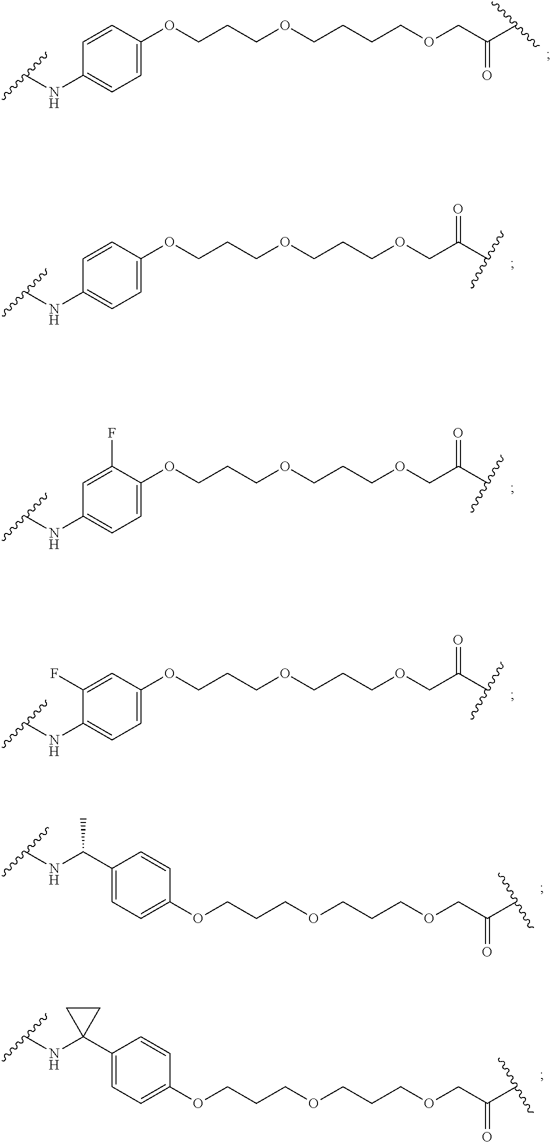

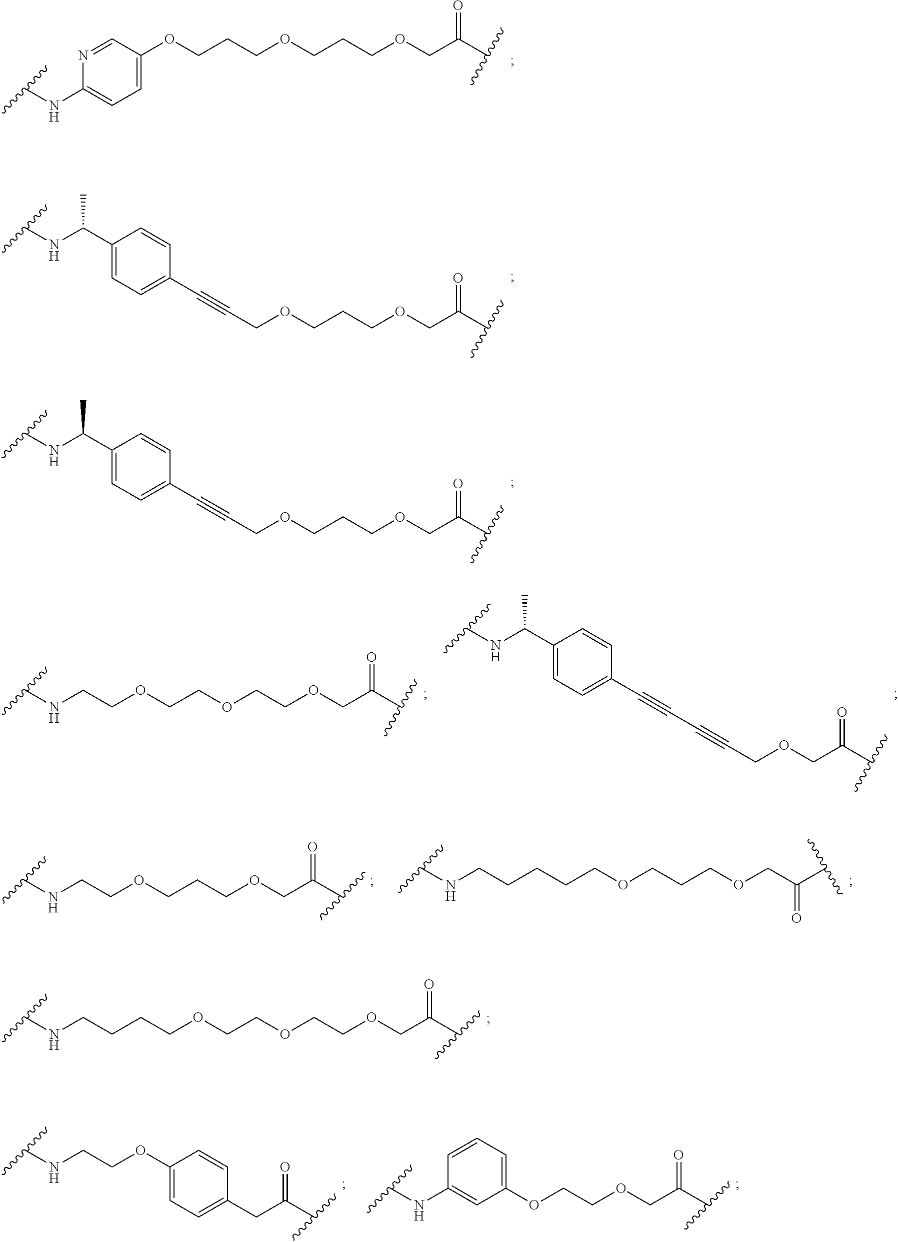

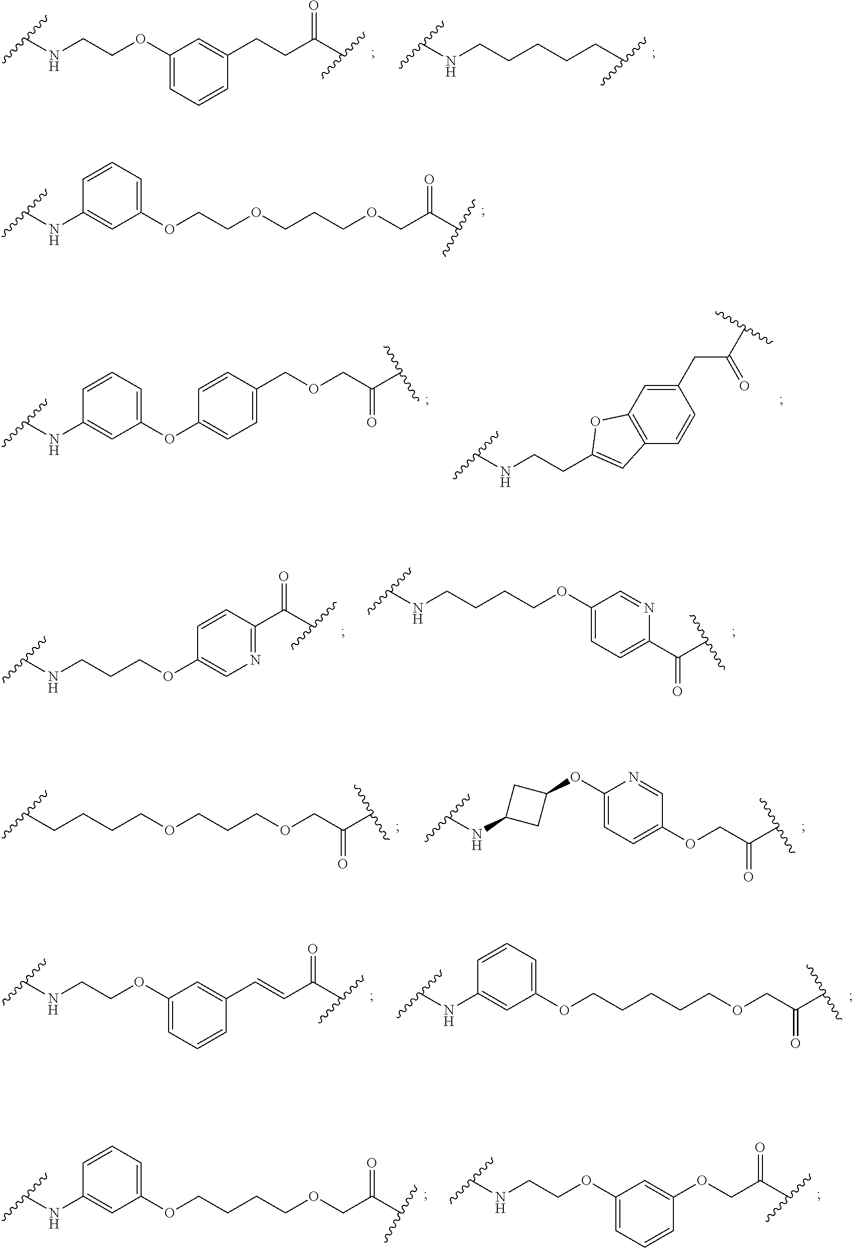

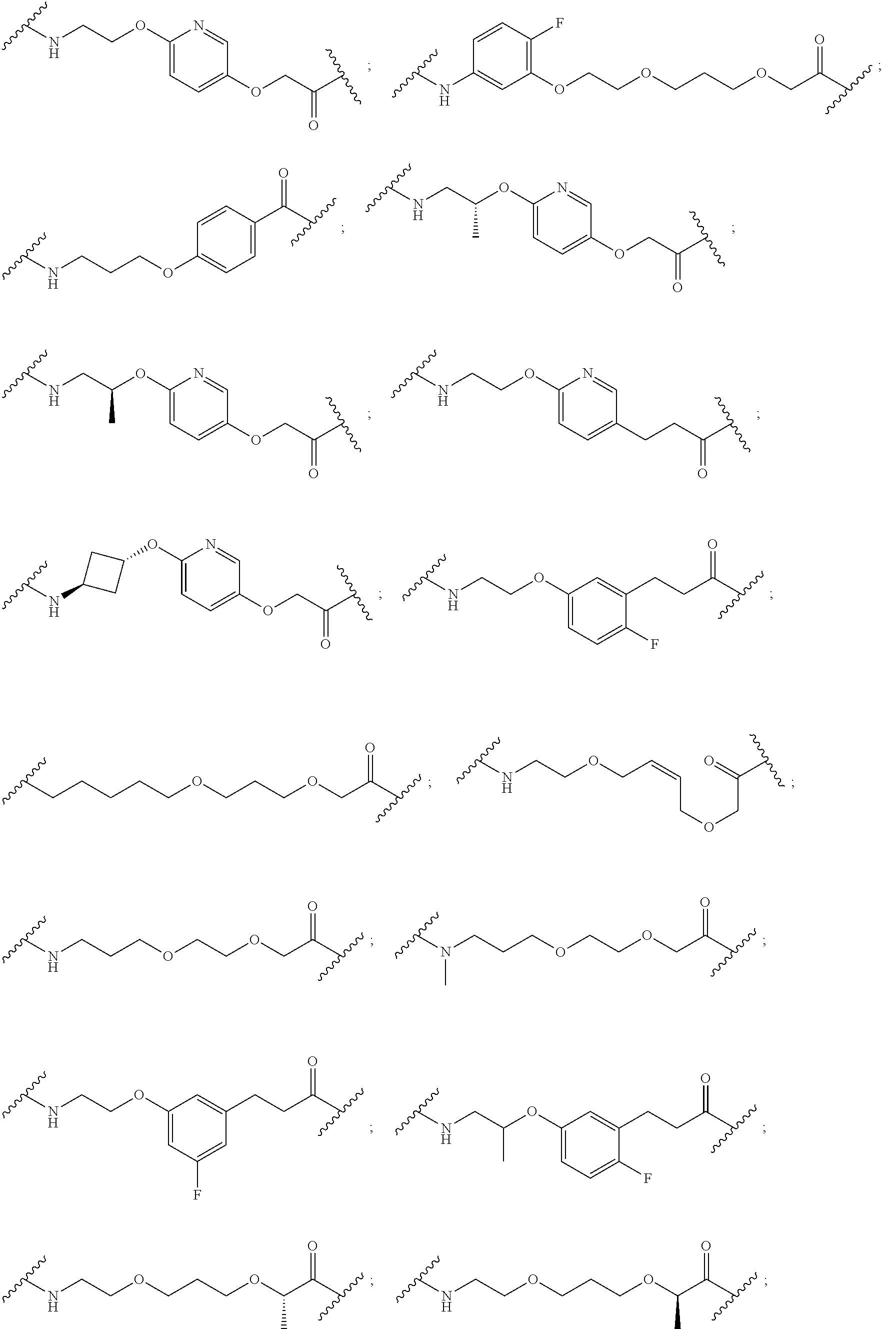

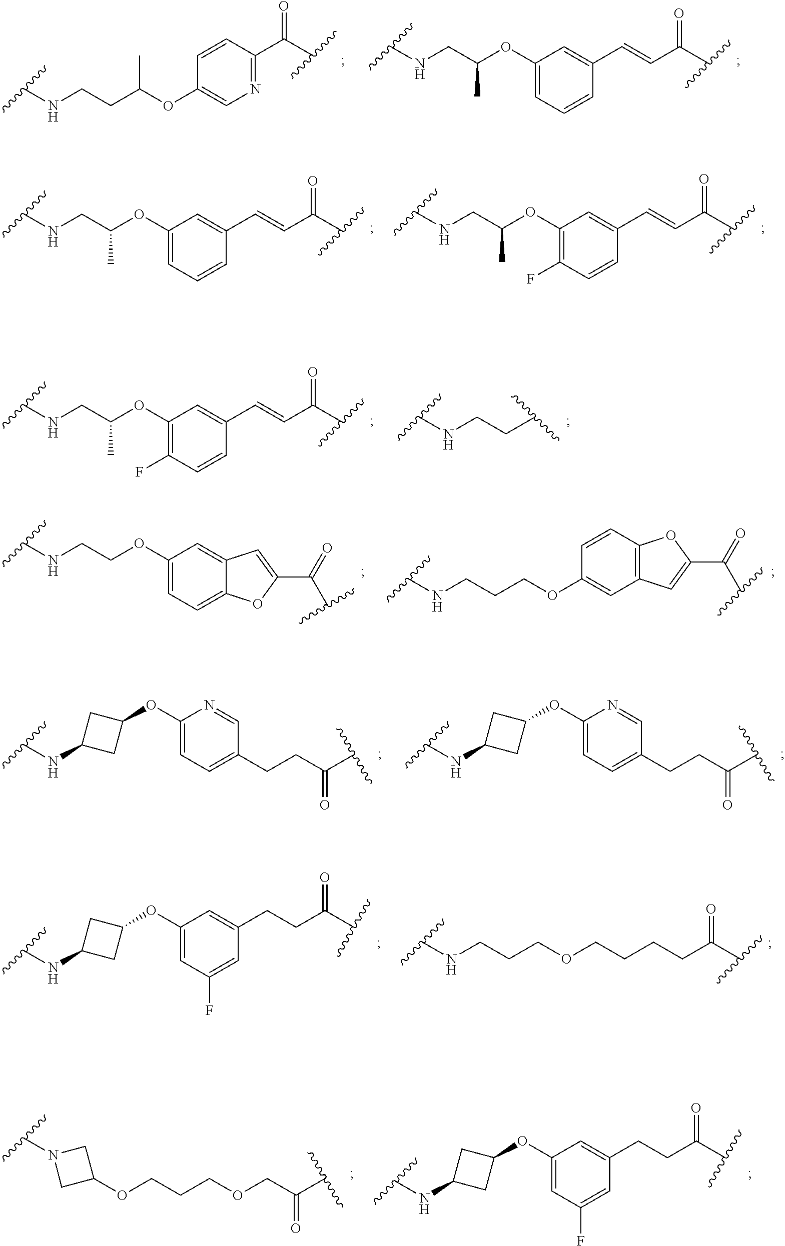

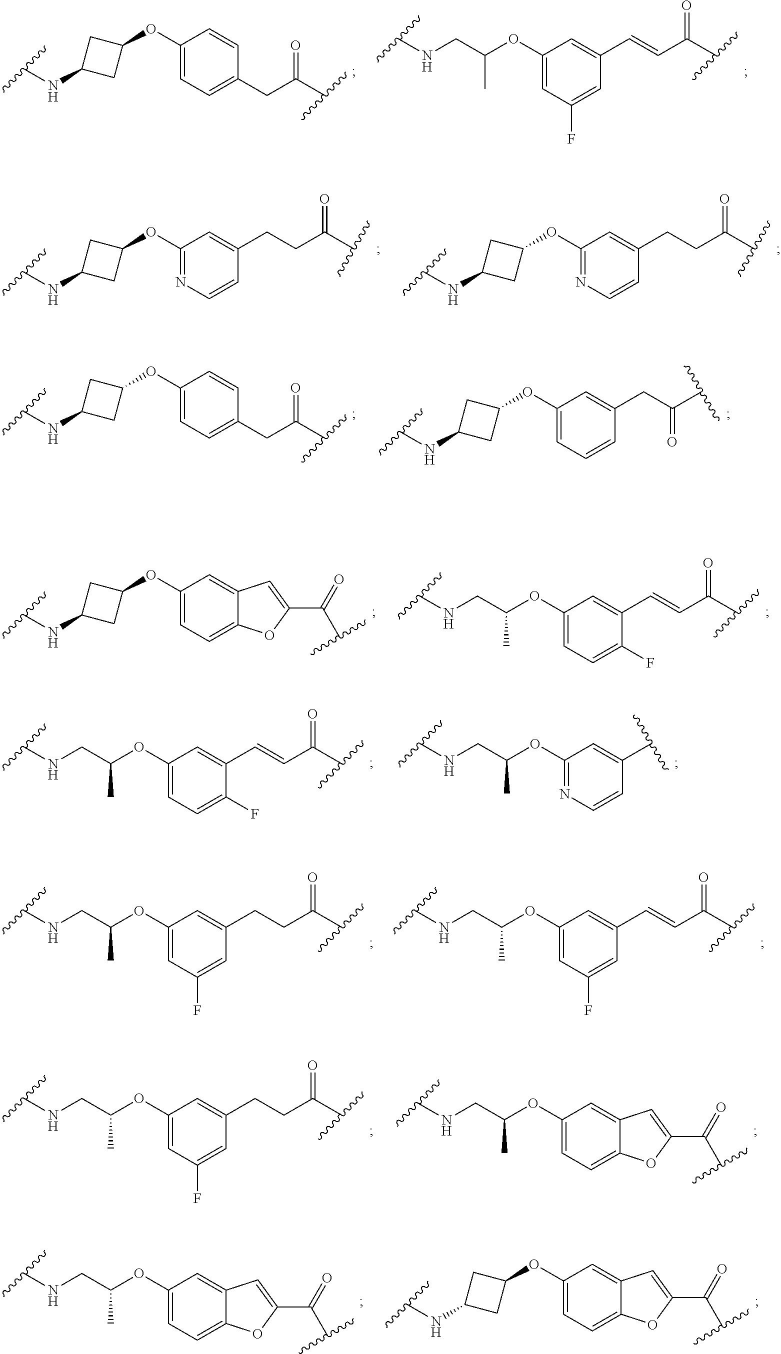

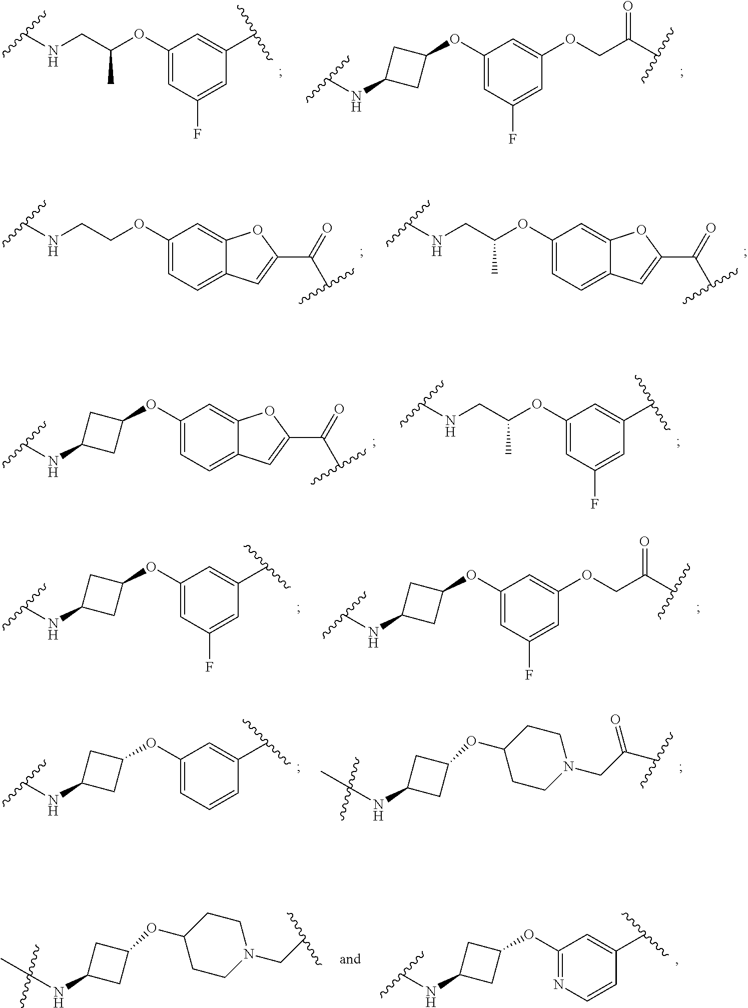

wherein each m and n is independently selected from 0, 1, 2, 3, 4, 5, 6, 7, 8, 9, 10, 11, 12, 13, 14, 15, 16, 17, 18, 19, or 20.

In any aspect or embodiment described herein, the unit A^{L} of linker (L) is selected from the group consisting of:

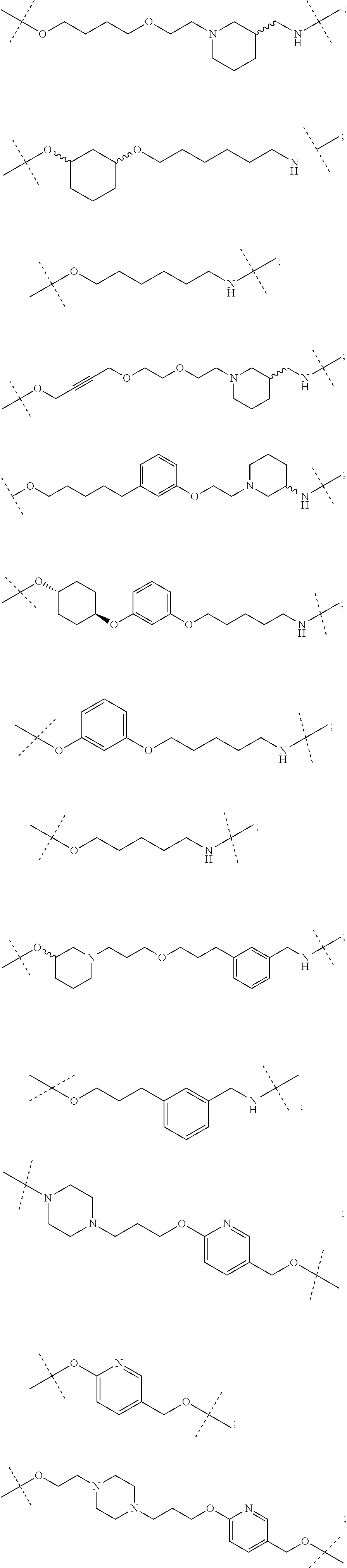

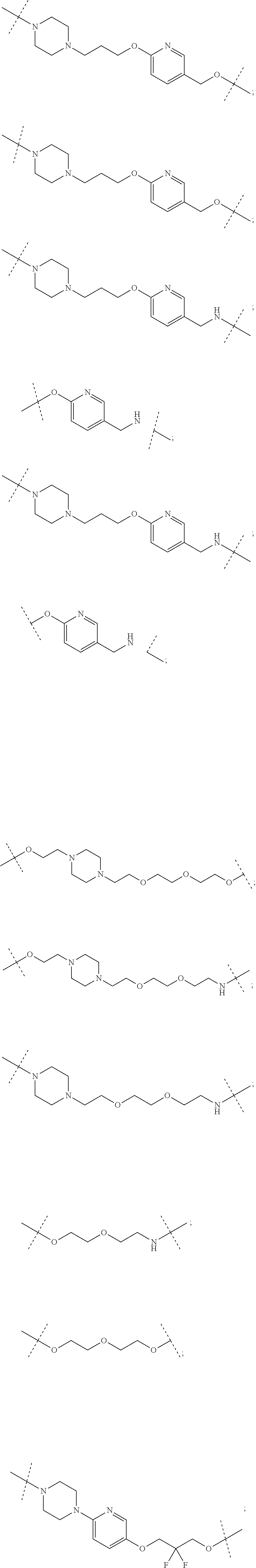

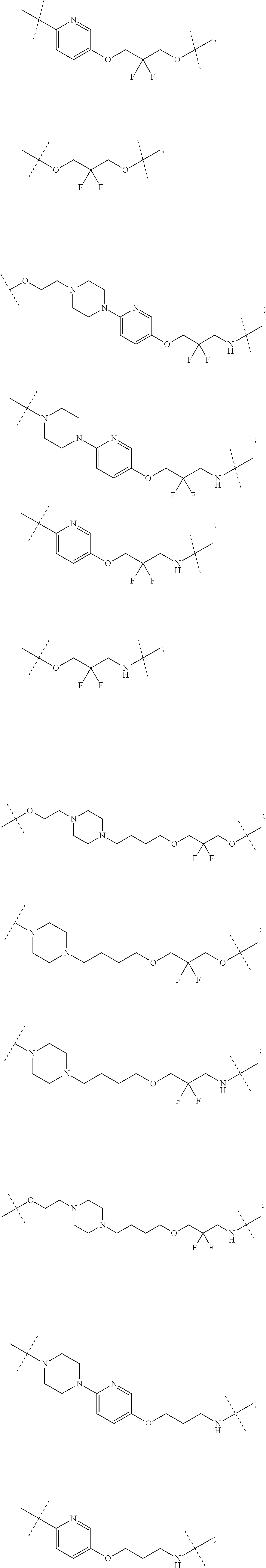

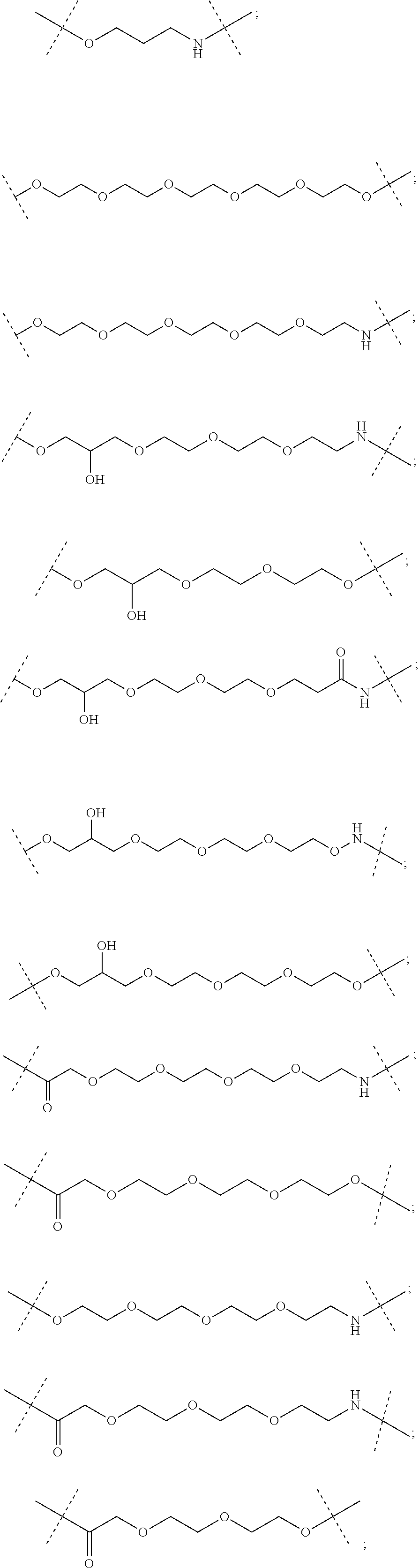

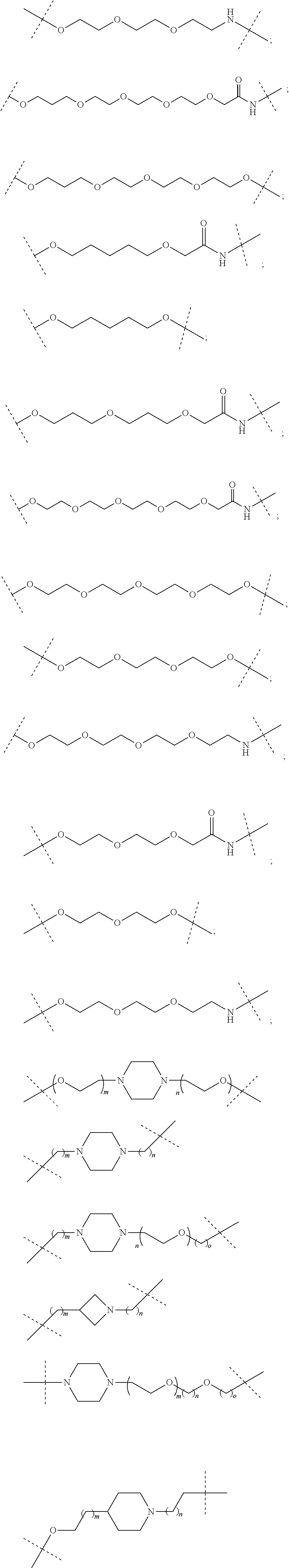

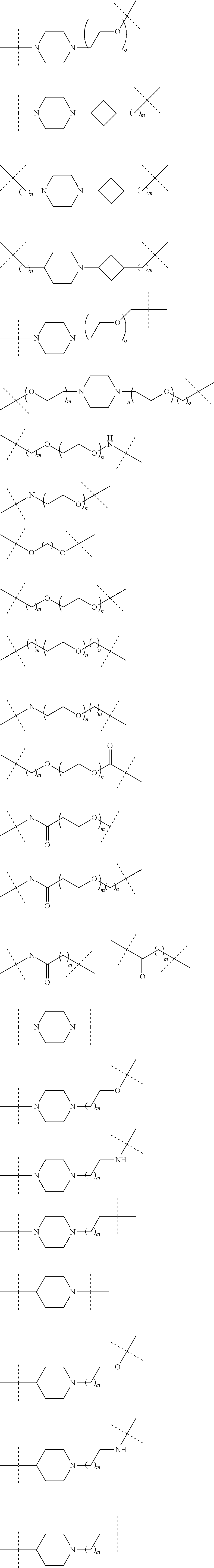

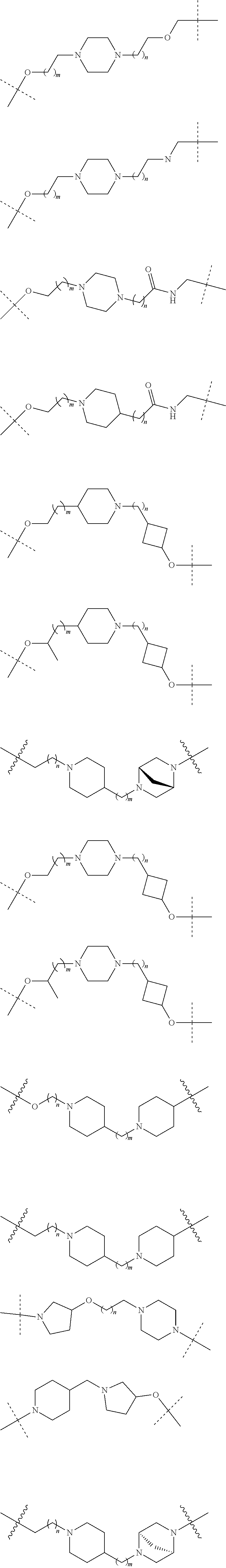

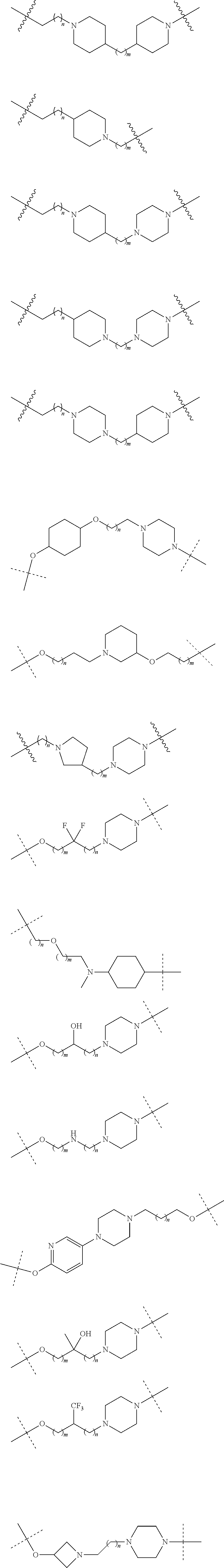

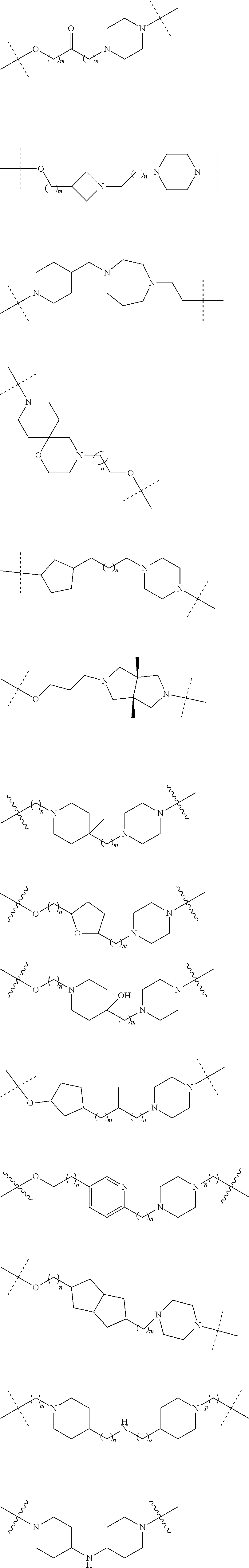

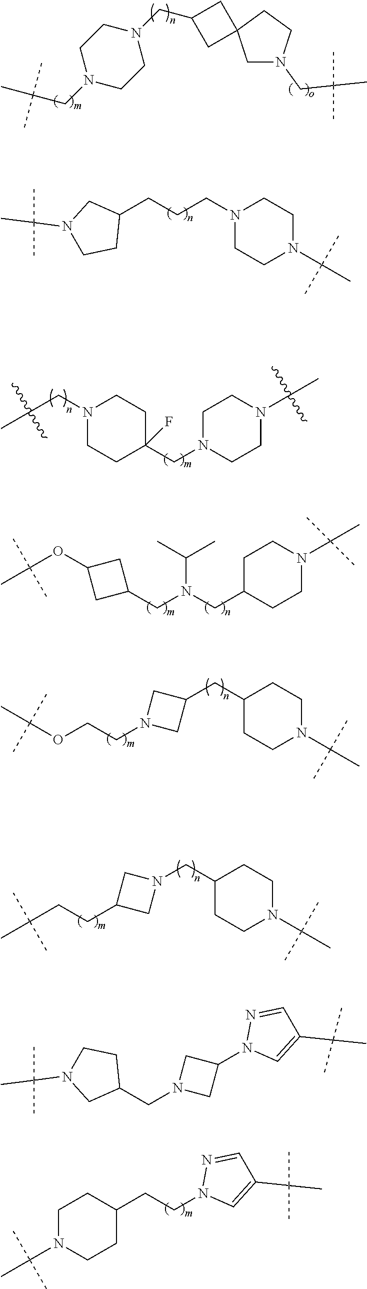

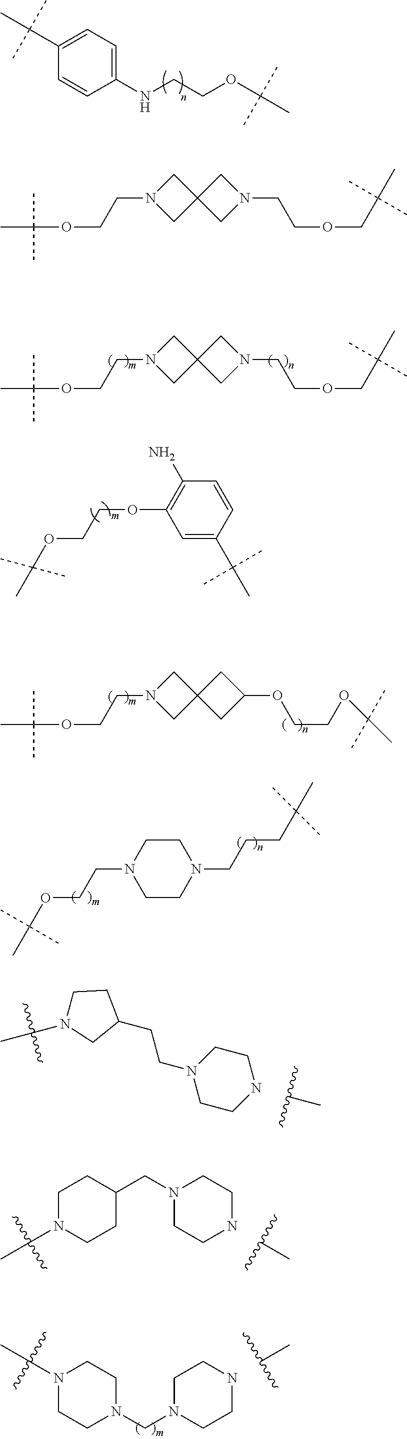

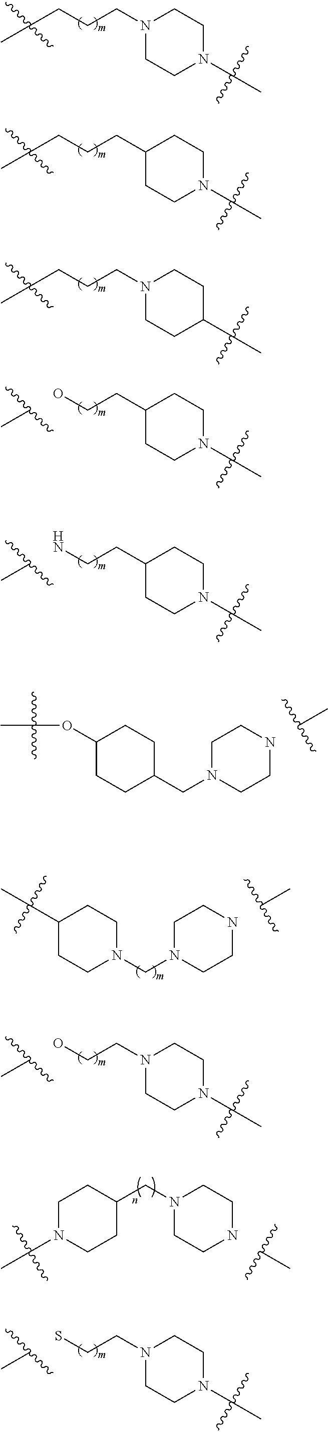

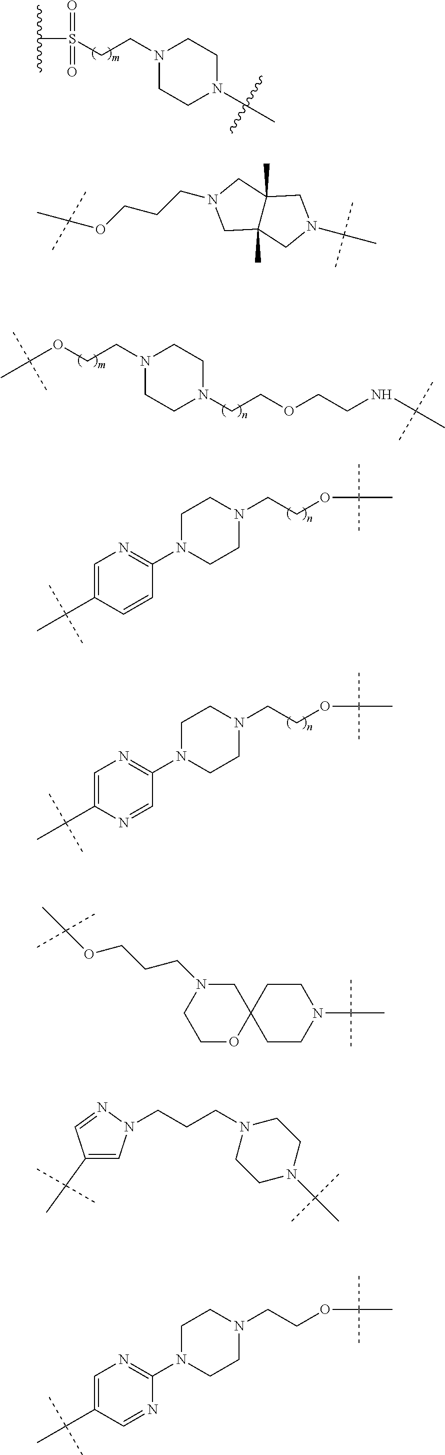

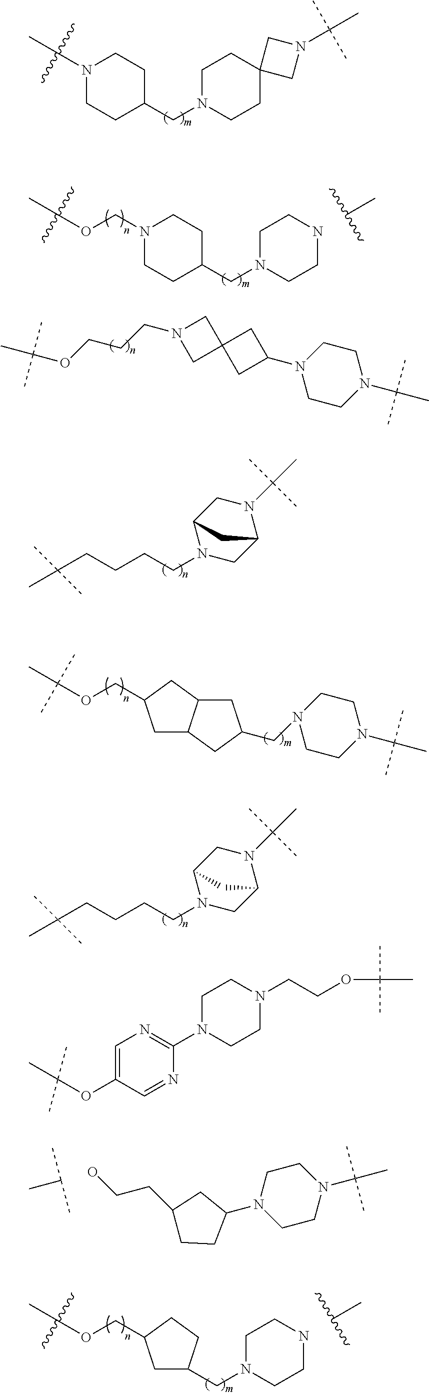

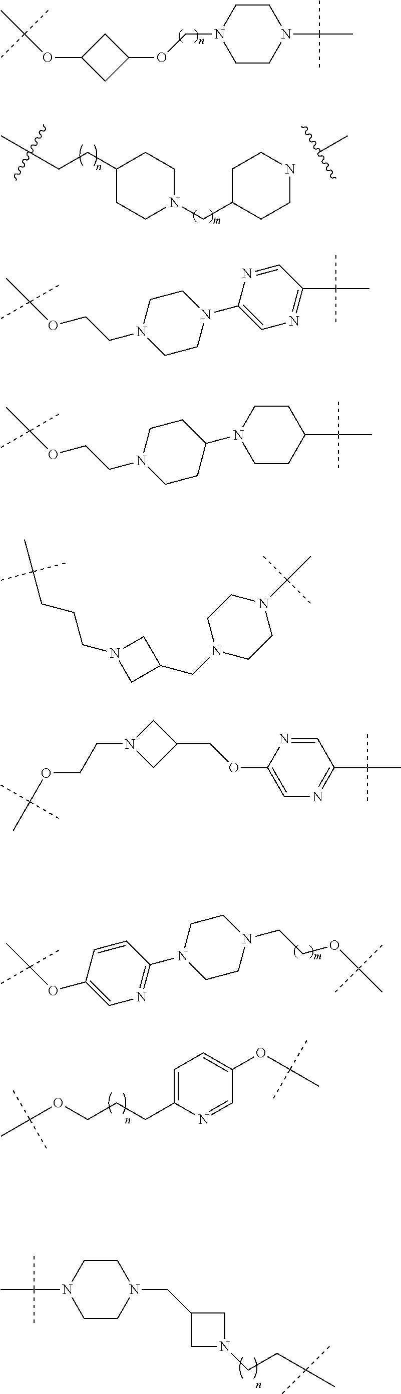

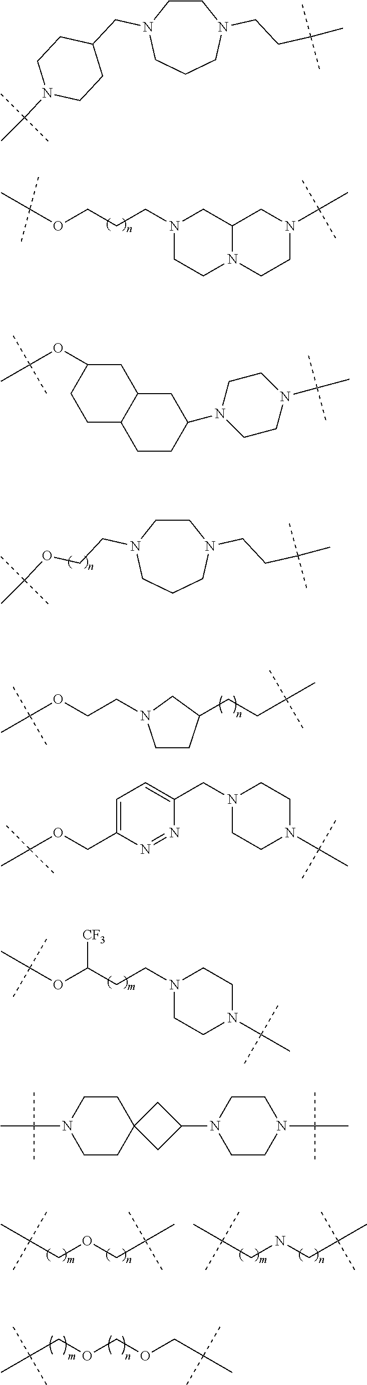

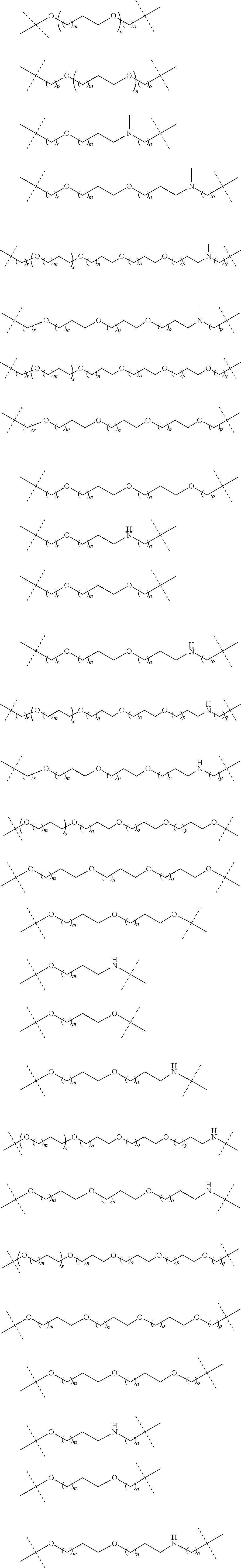

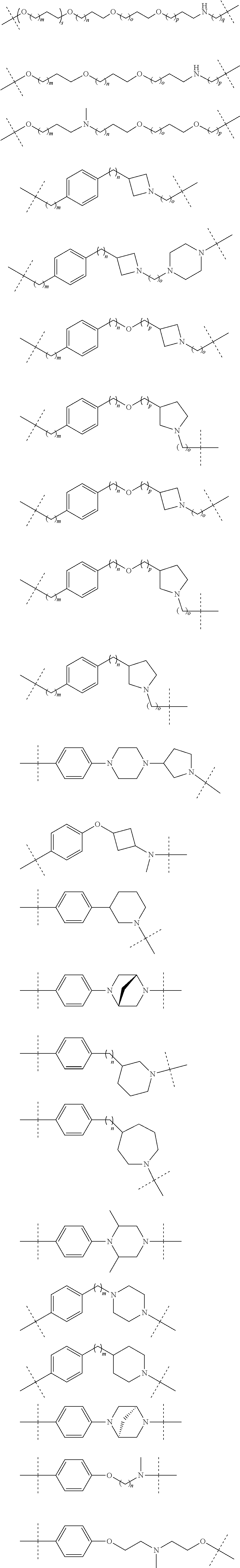

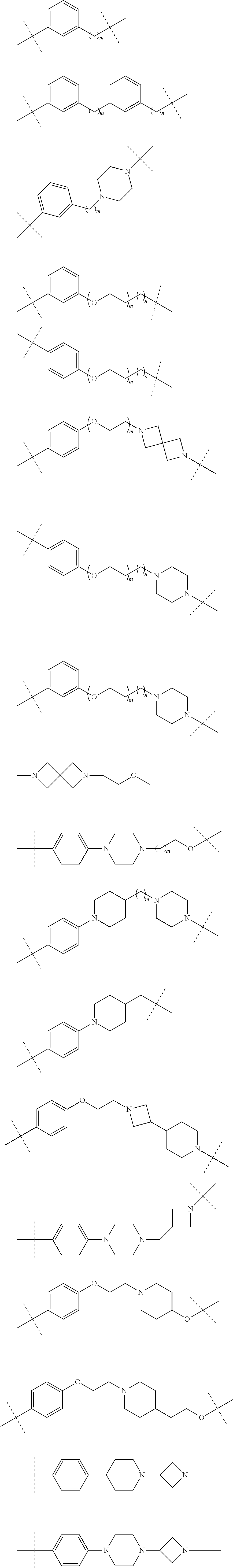

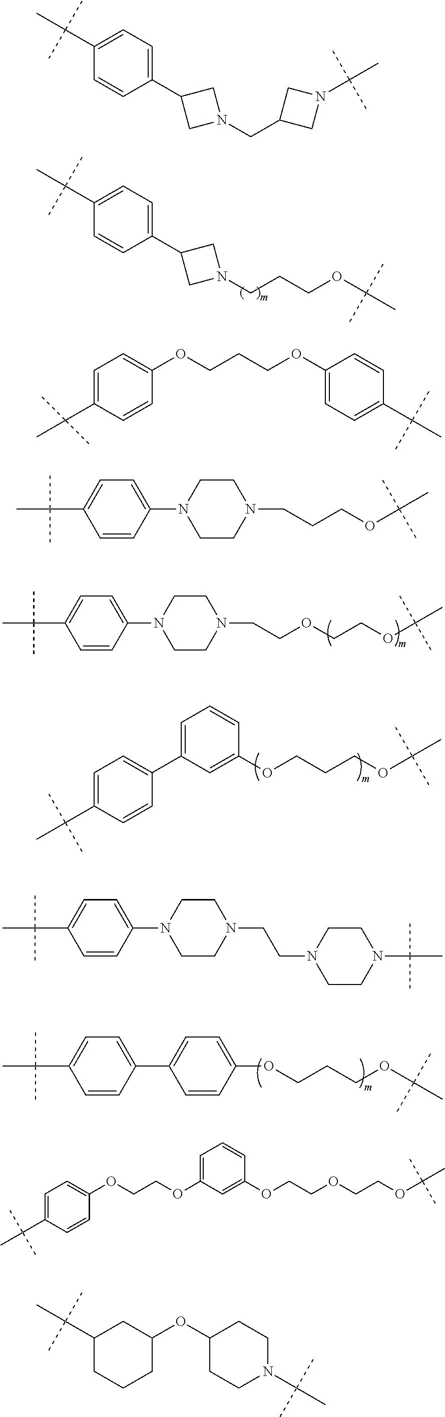

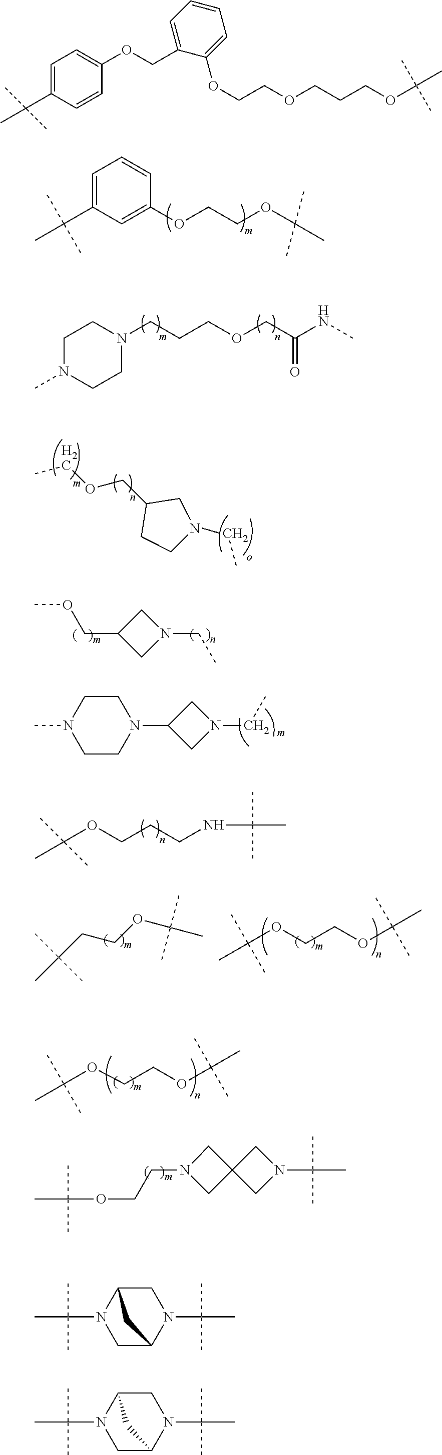

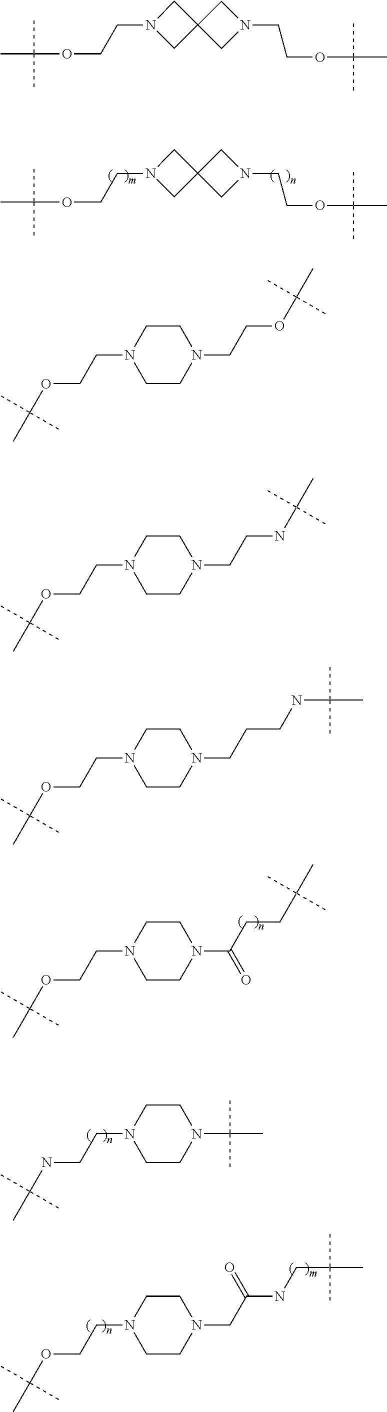

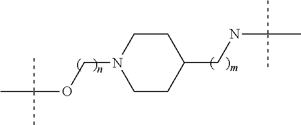

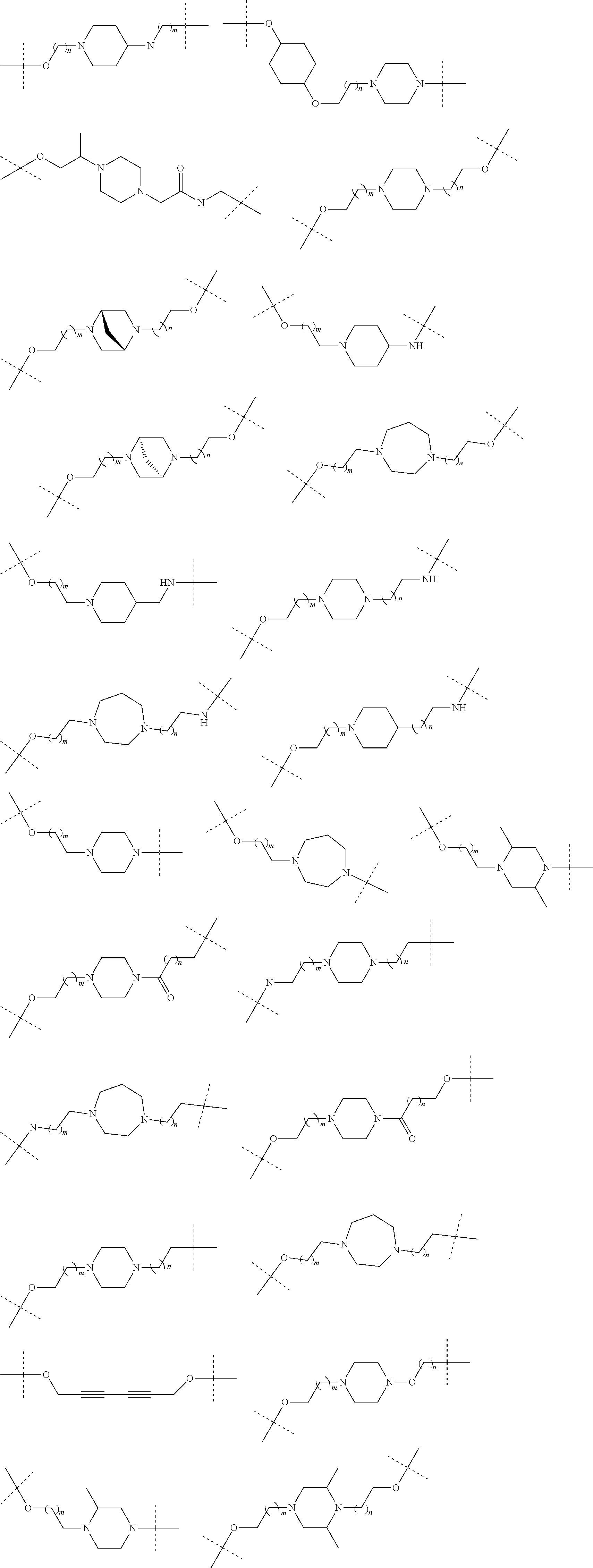

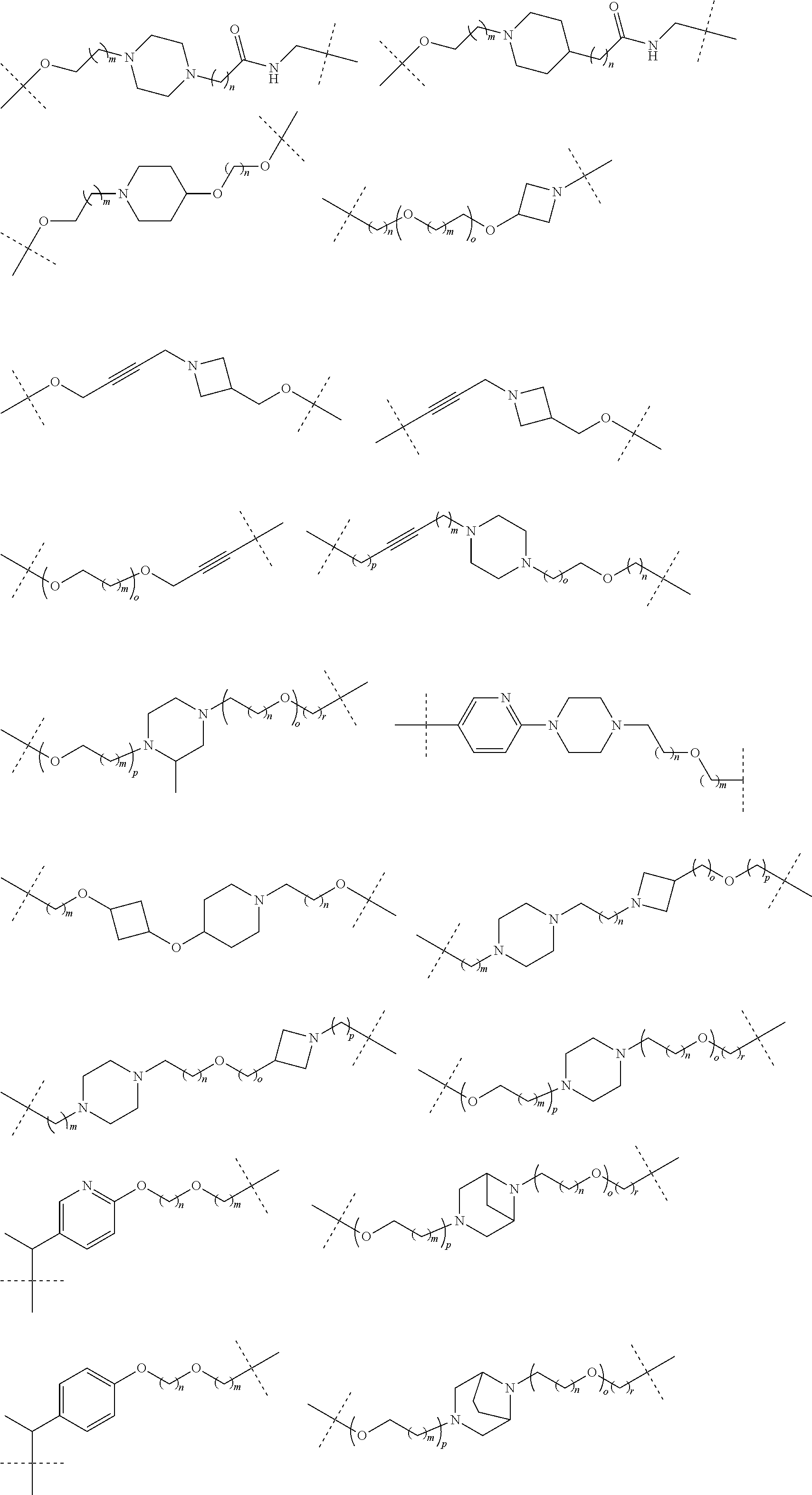

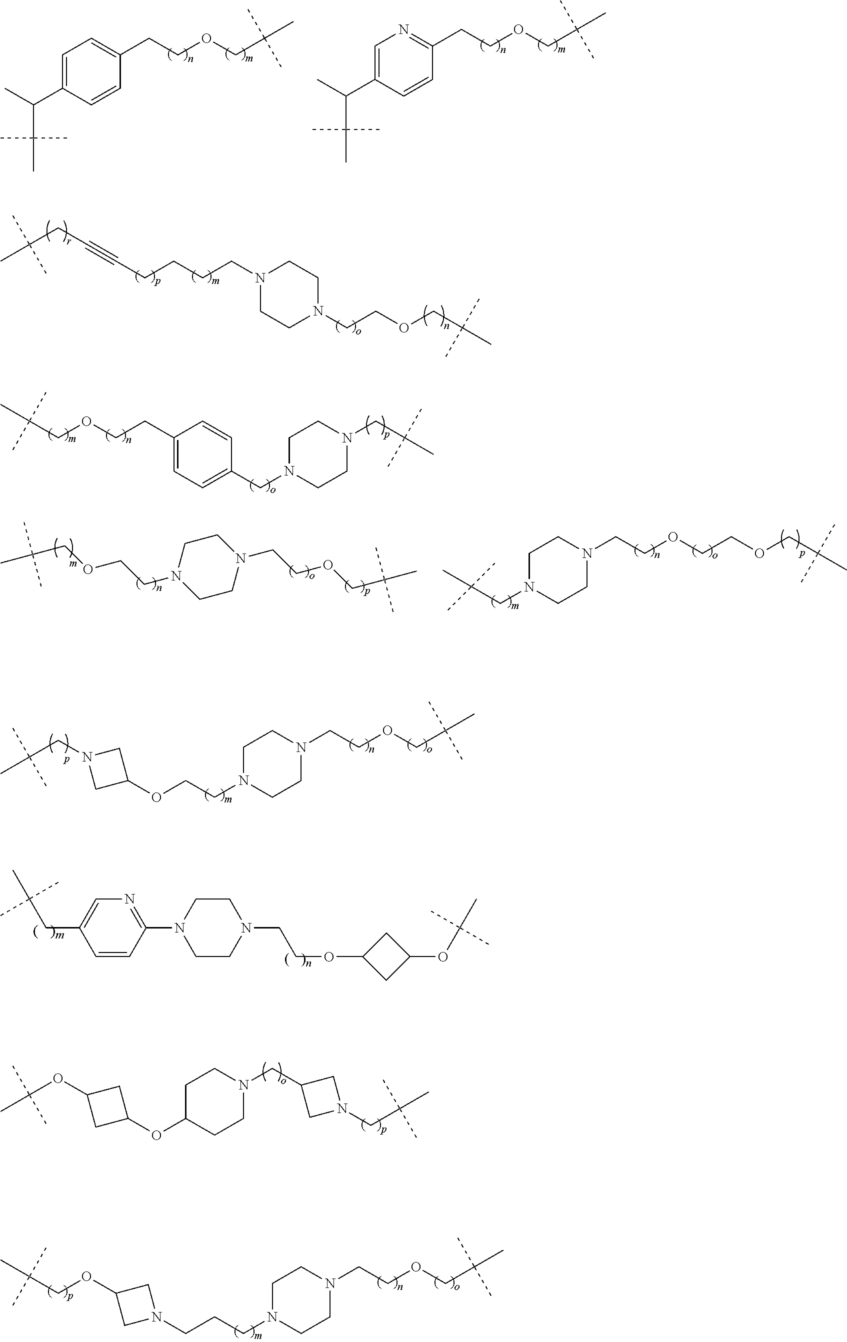

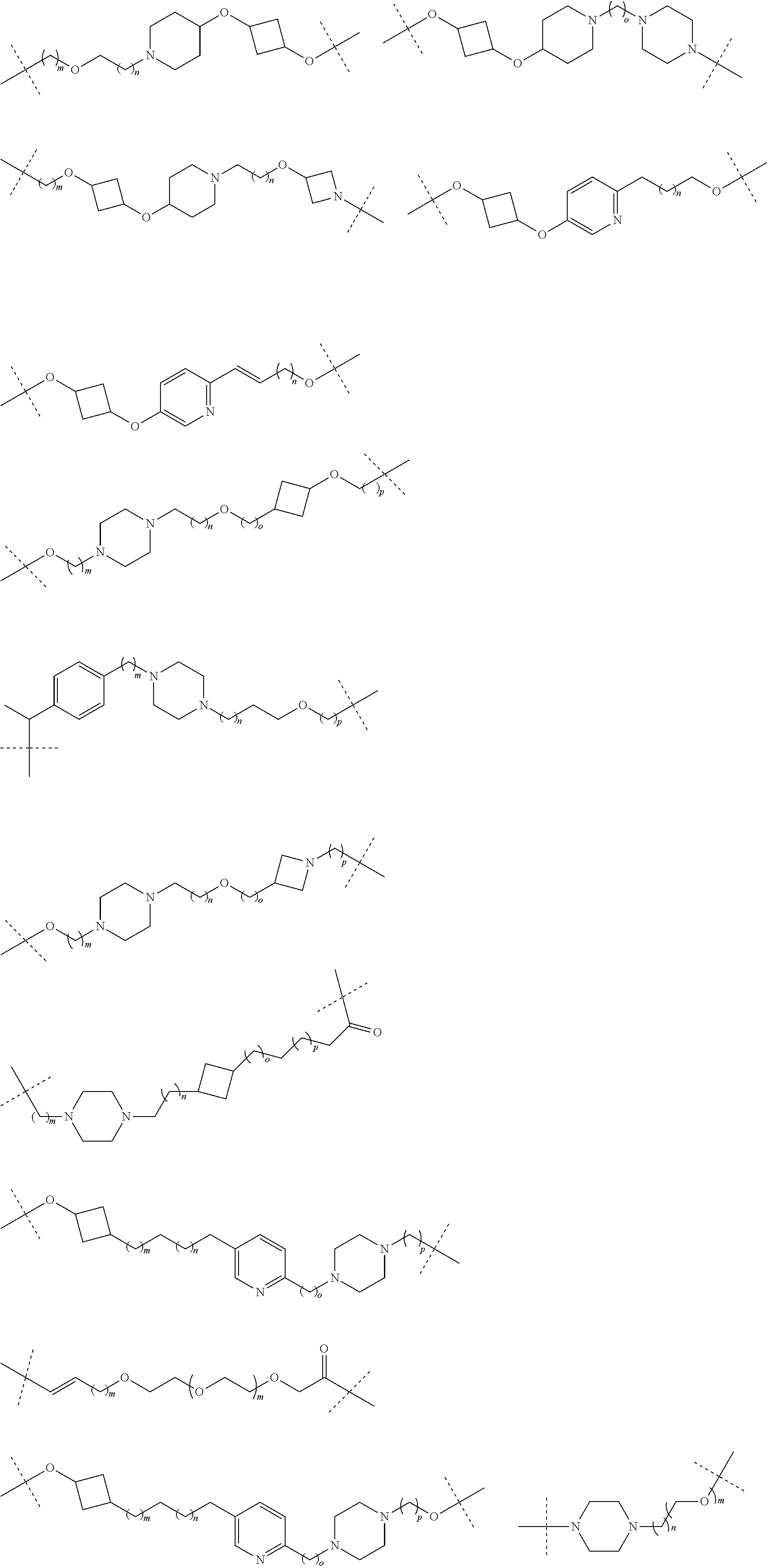

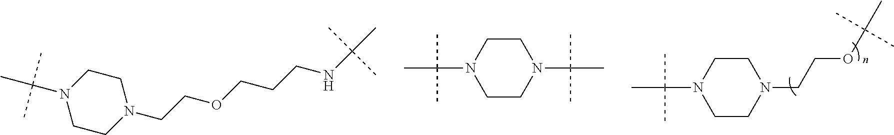

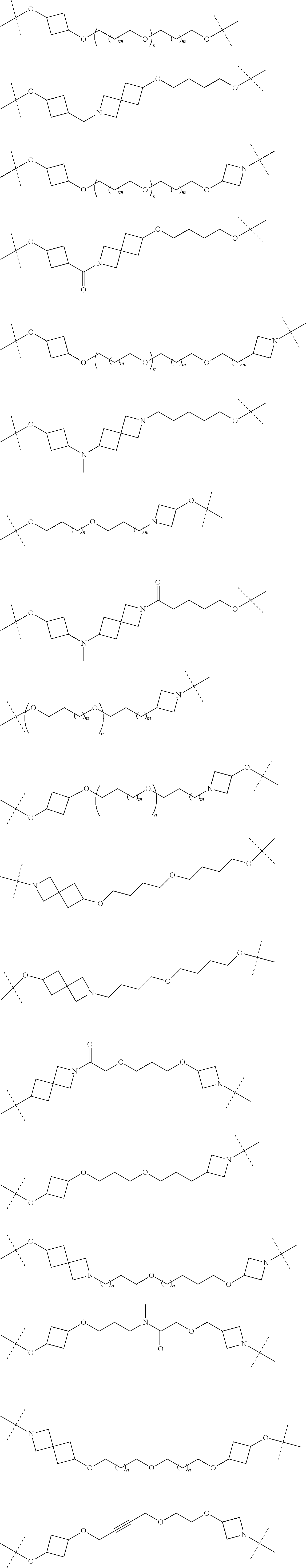

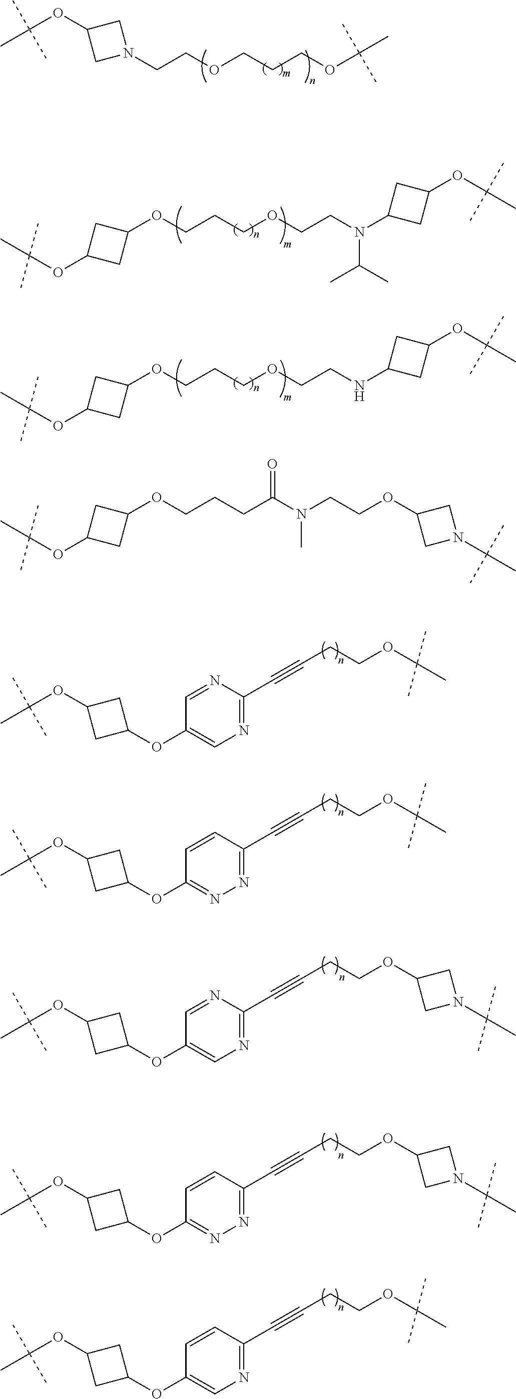

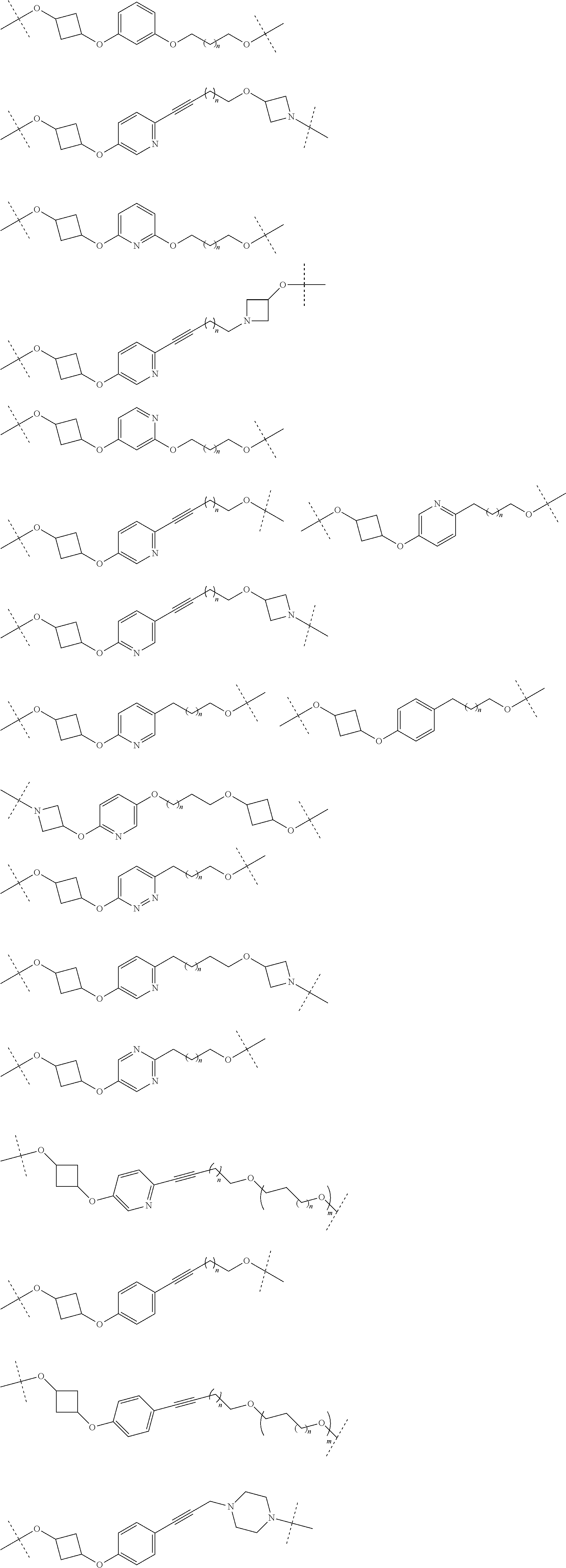

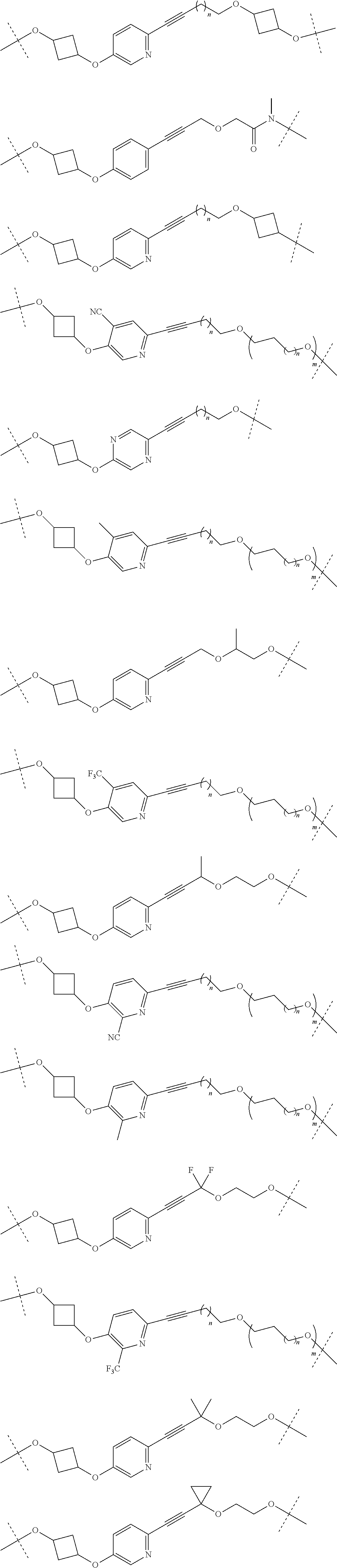

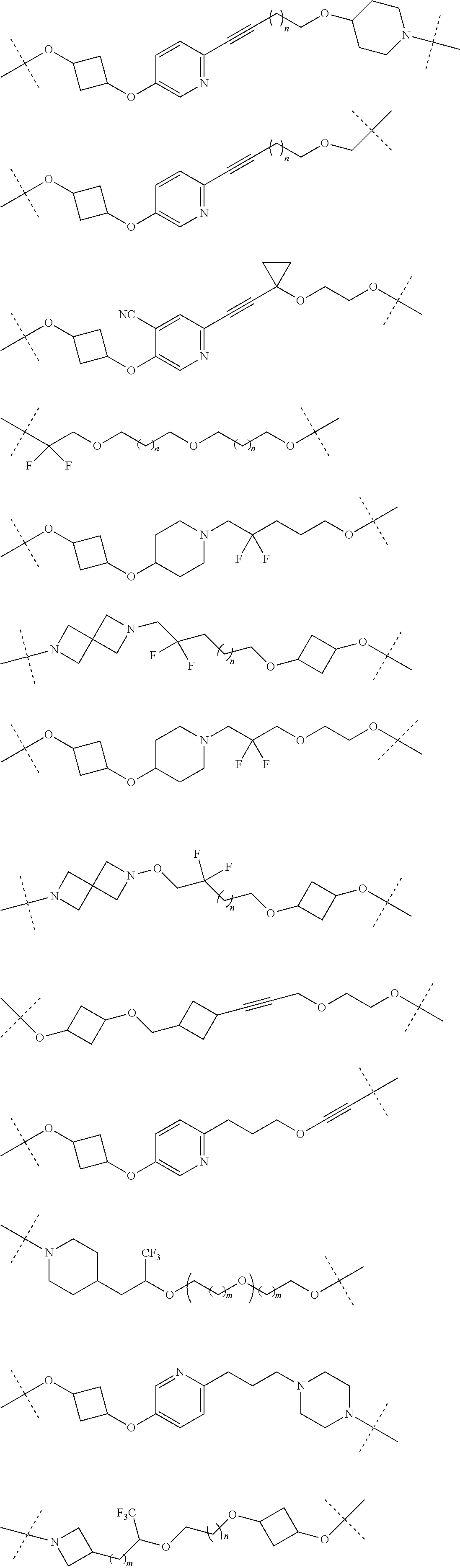

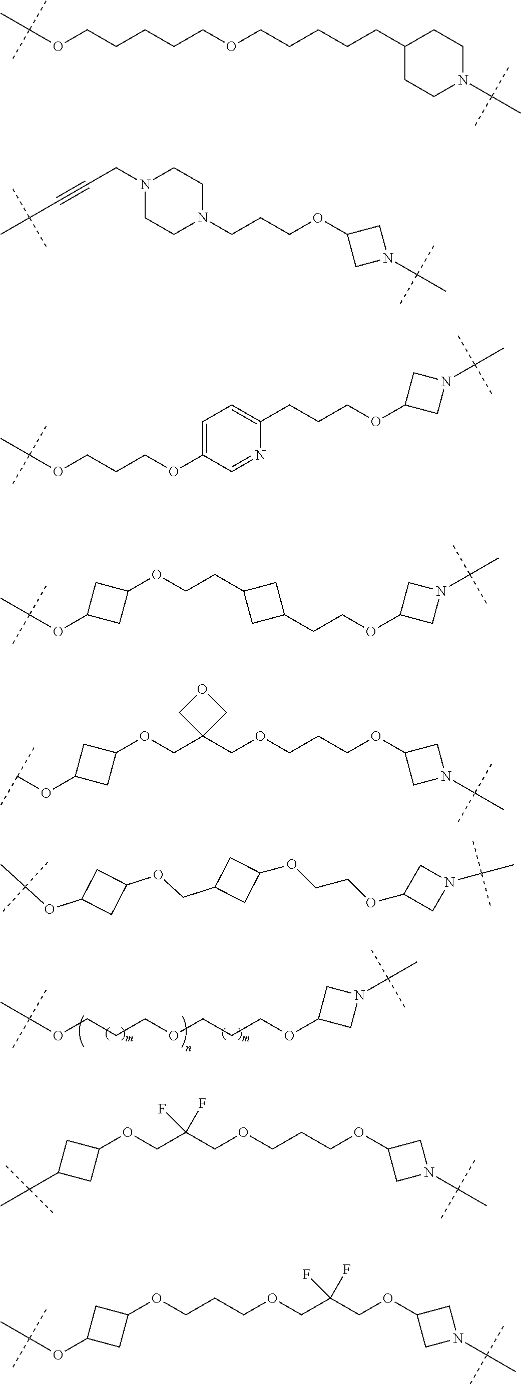

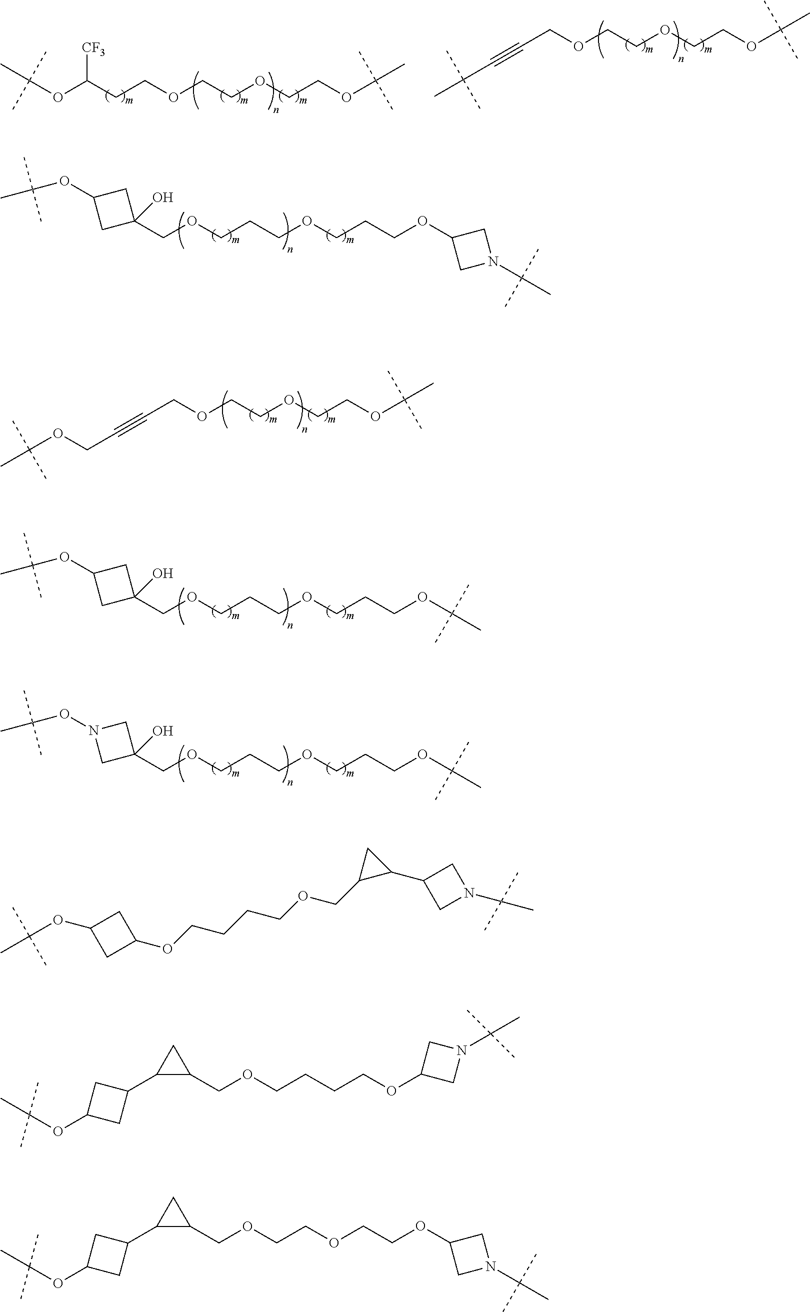

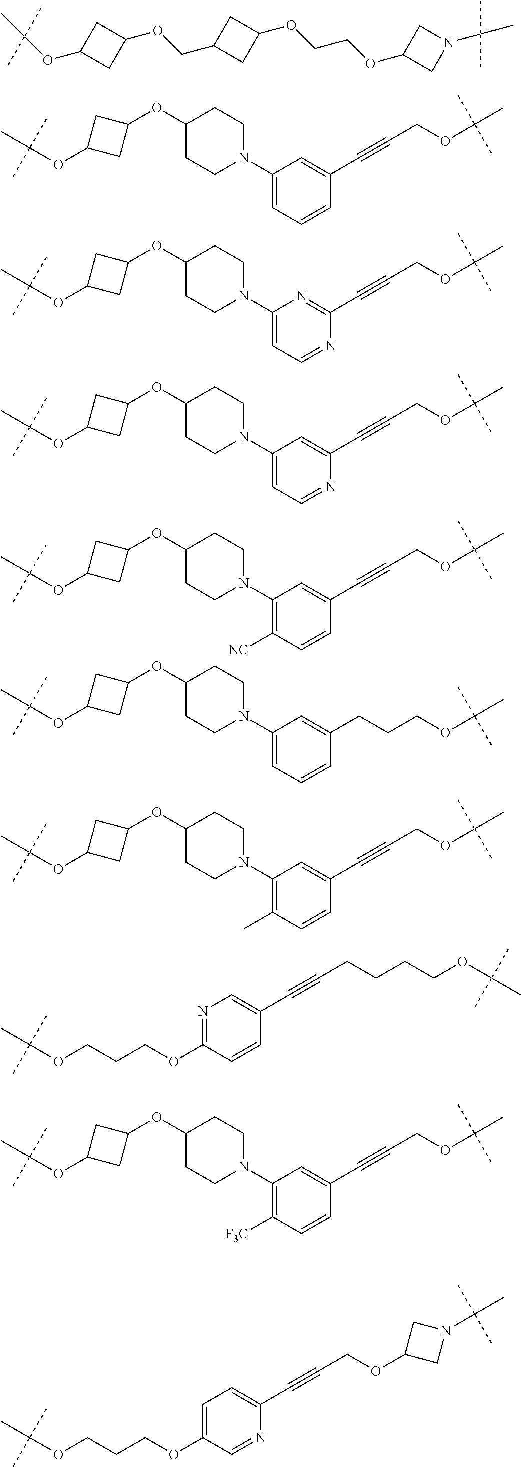

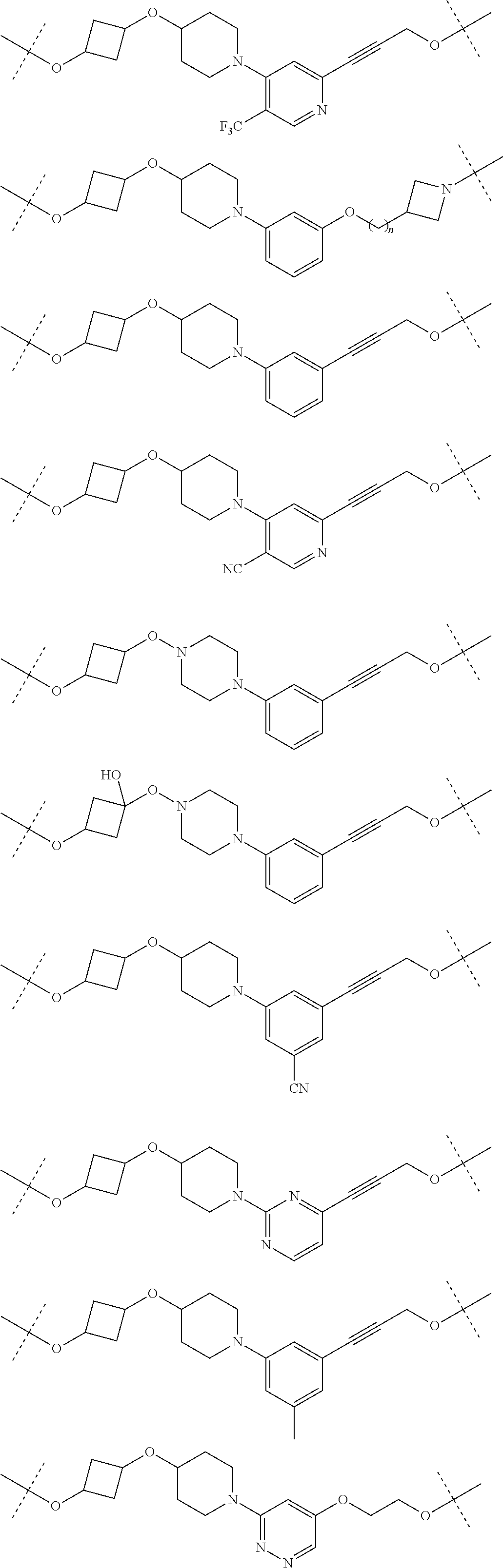

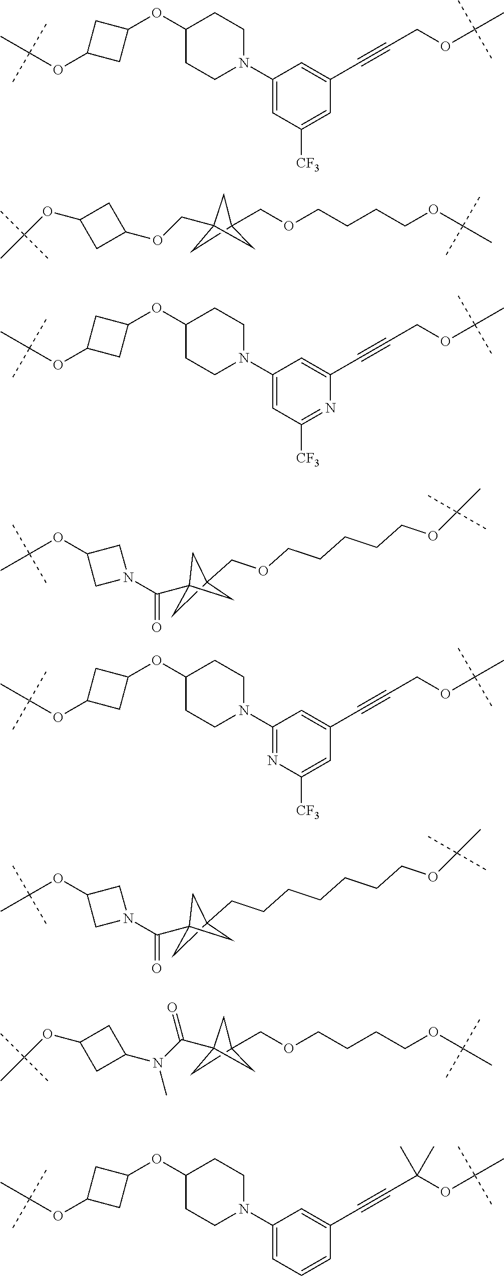

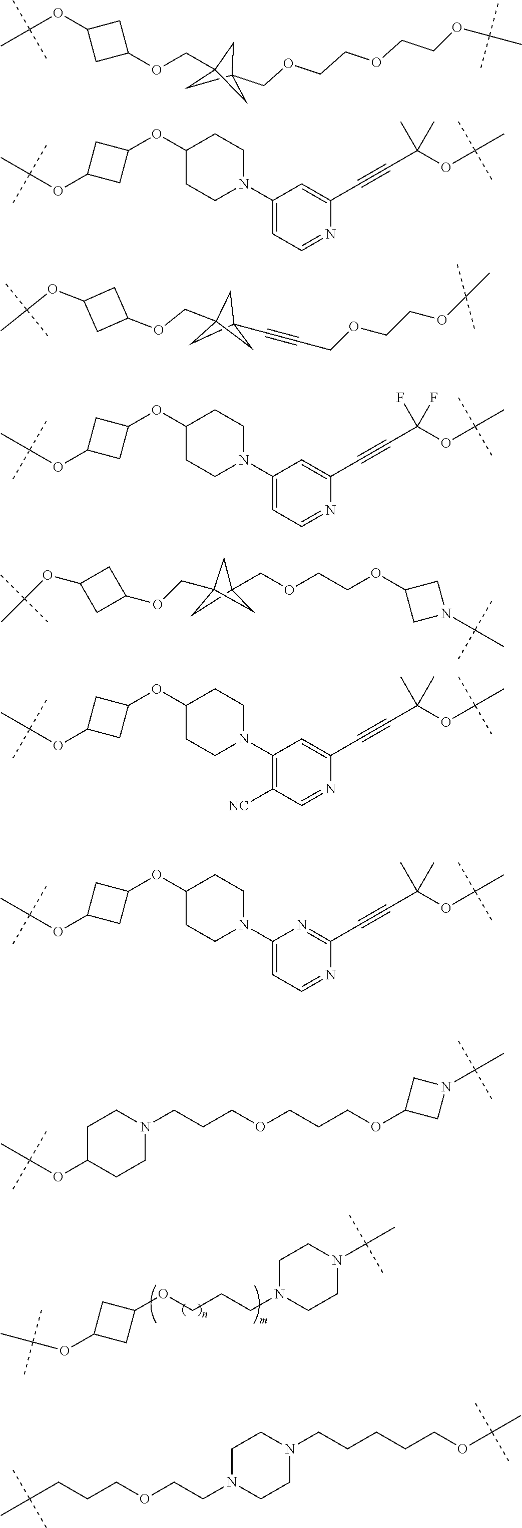

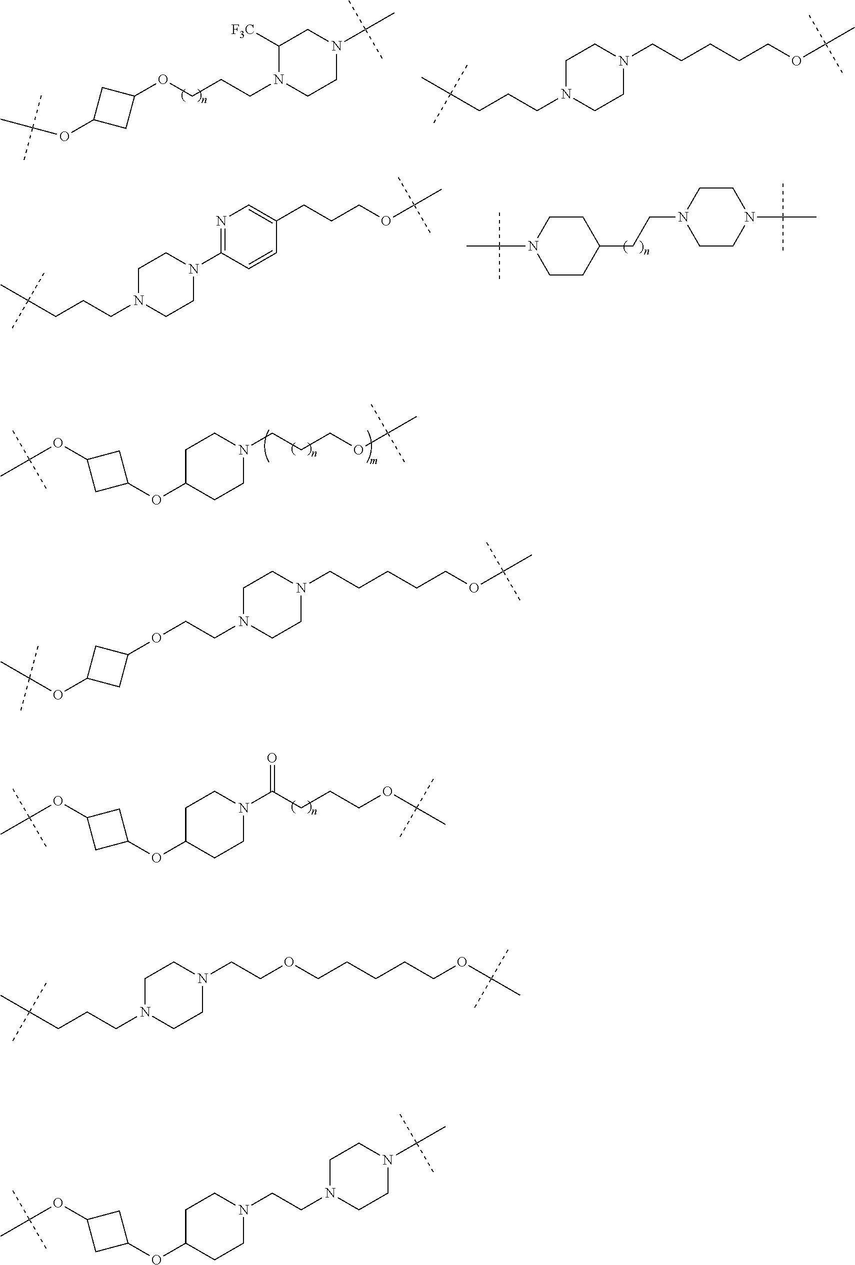

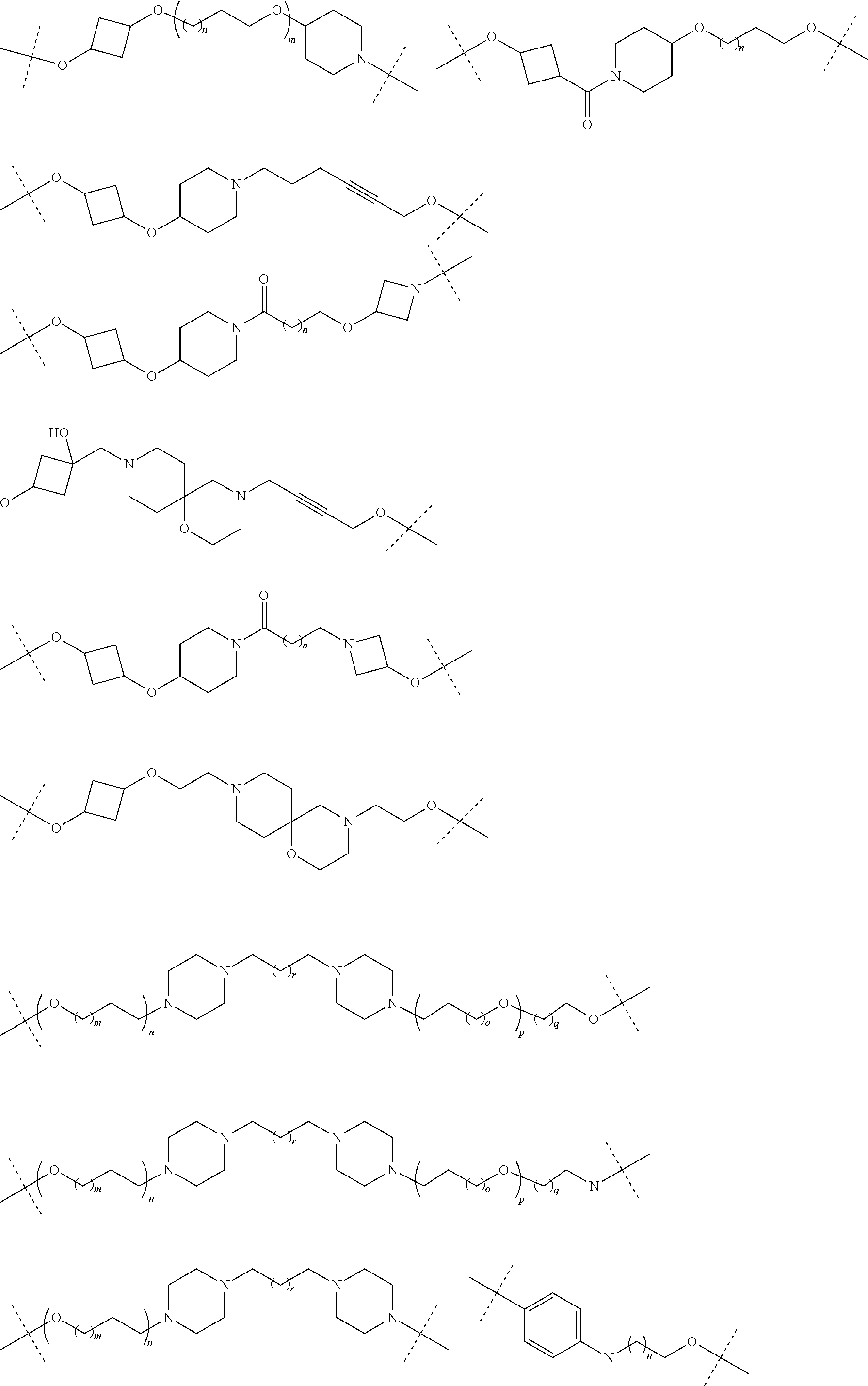

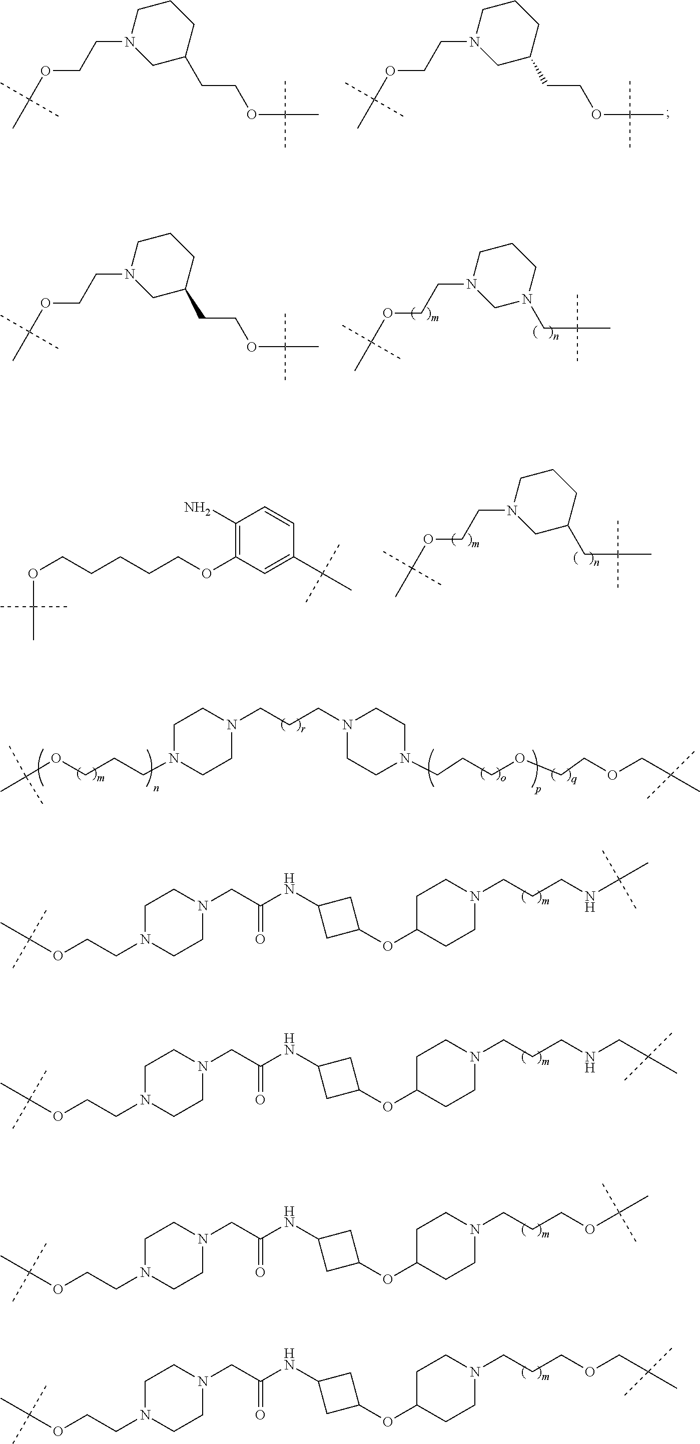

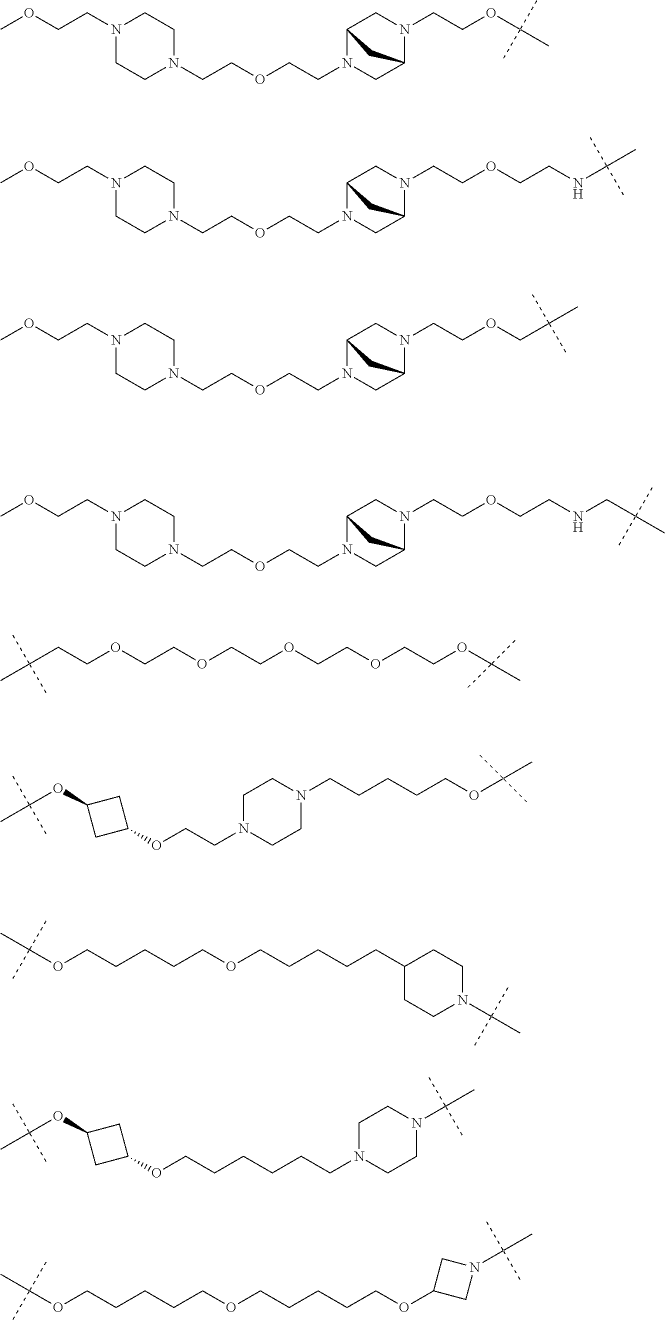

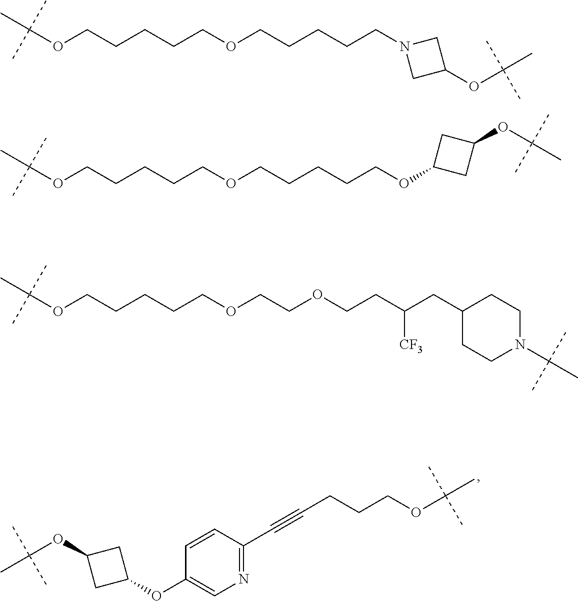

wherein each m, n, o, p, q, r, and s is independently 0, 1, 2, 3, 4, 5, 6, 7, 8, 9, 10, 11, 12, 13, 14, 15, 16, 17, 18, 19, or 20.

In any aspect or embodiment described herein, the unit A^{L} of linker (L) is selected from the group consisting of:

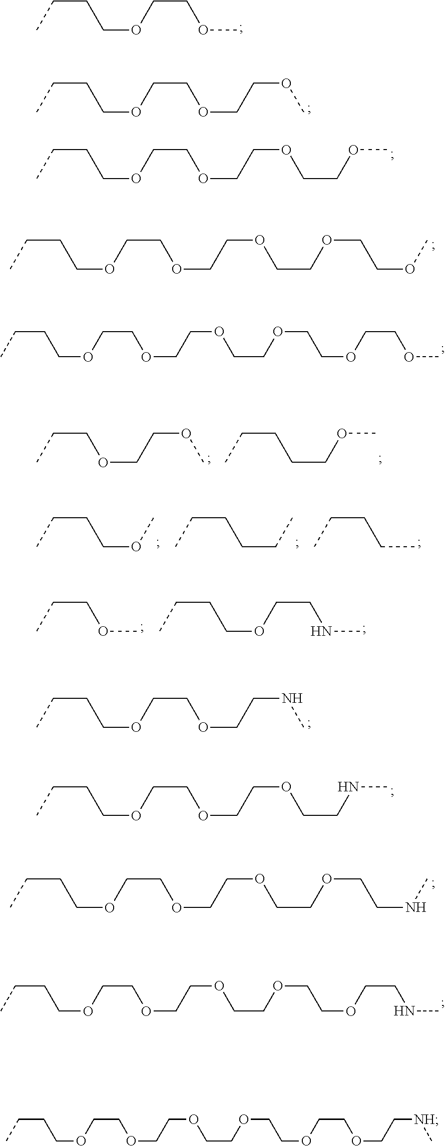

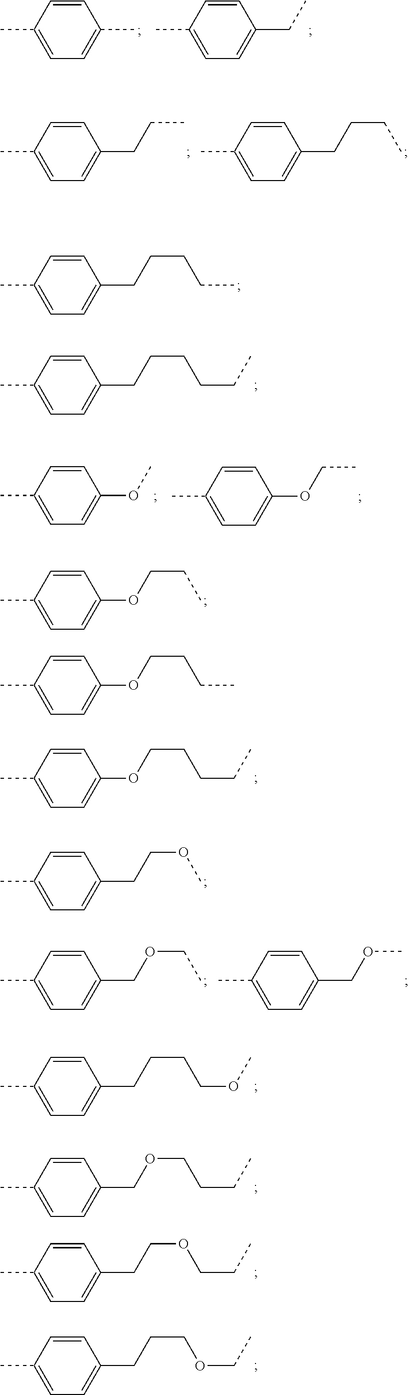

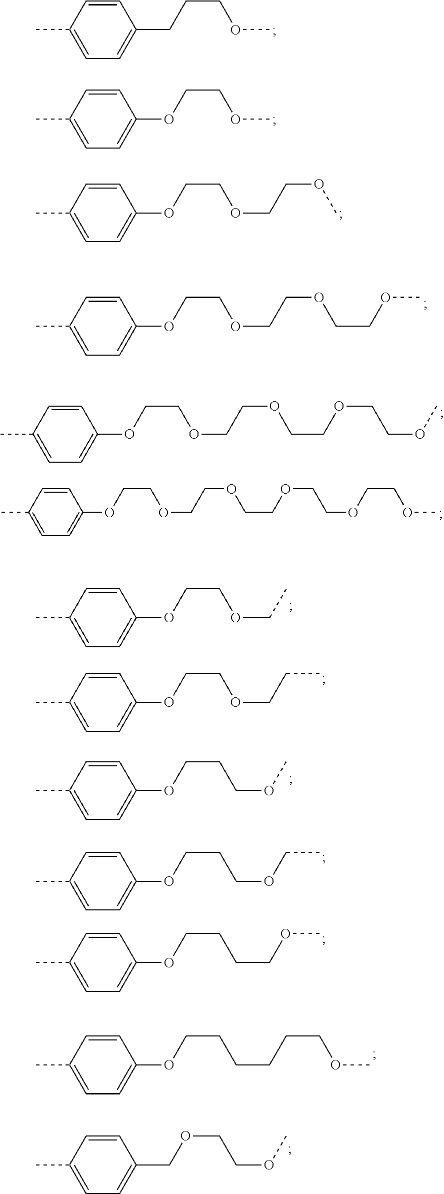

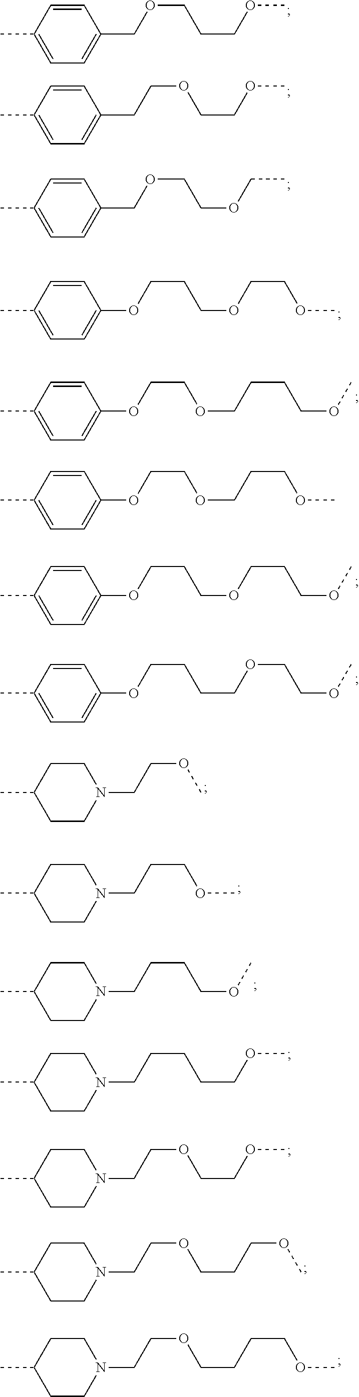

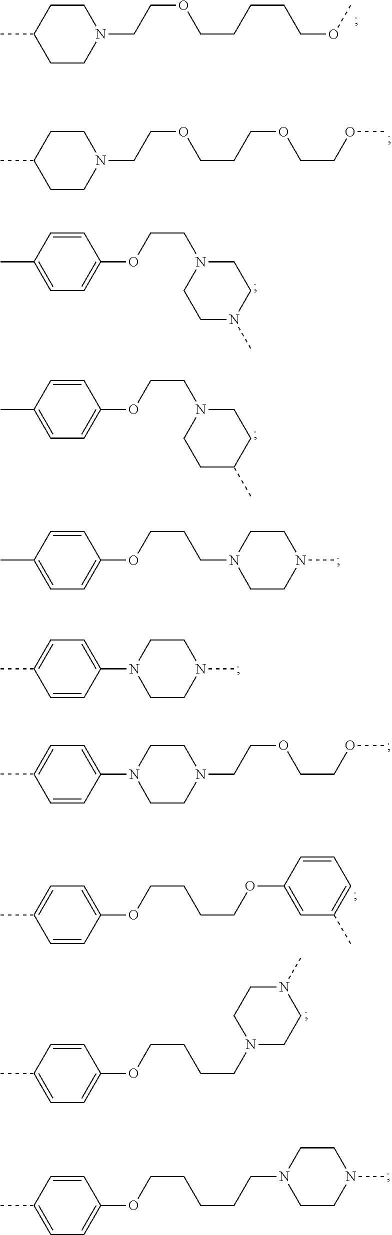

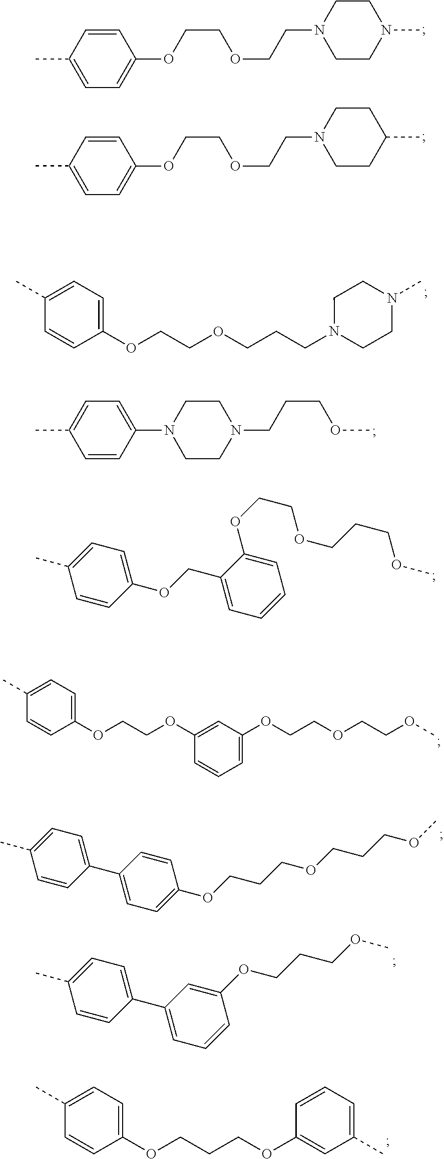

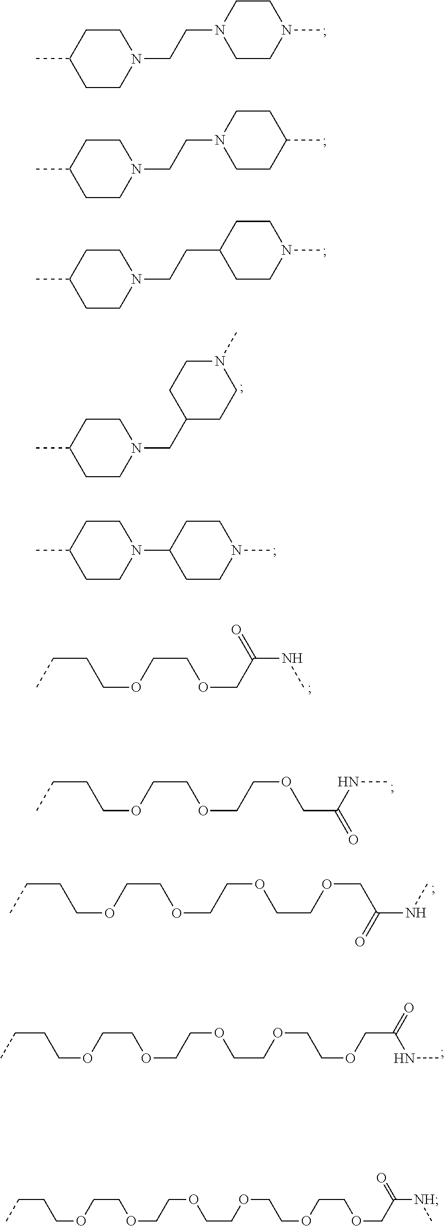

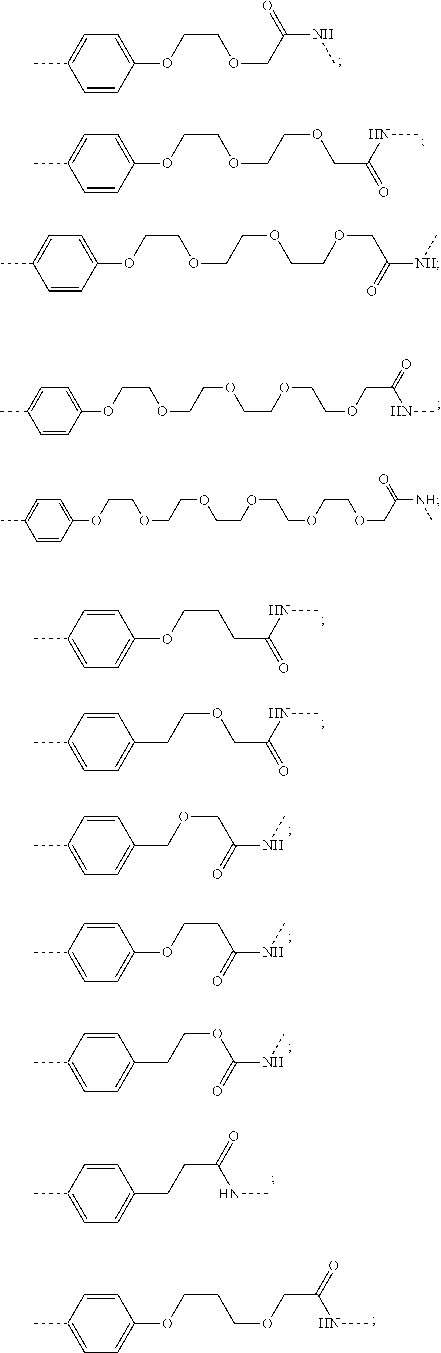

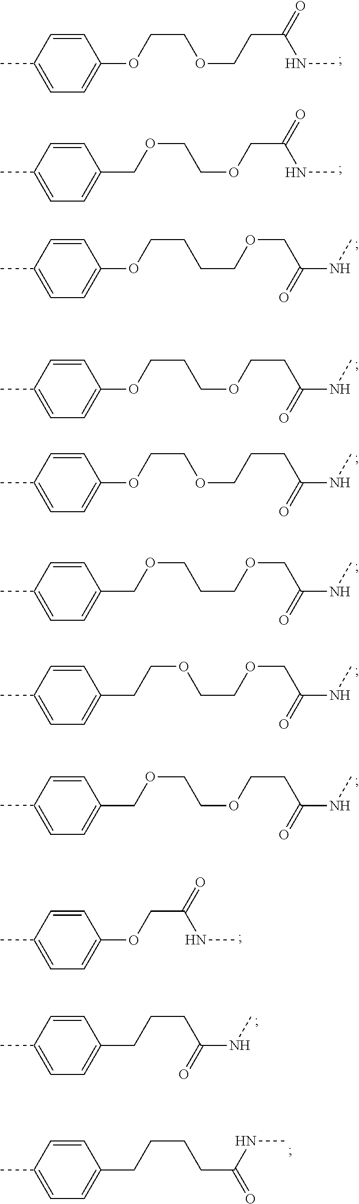

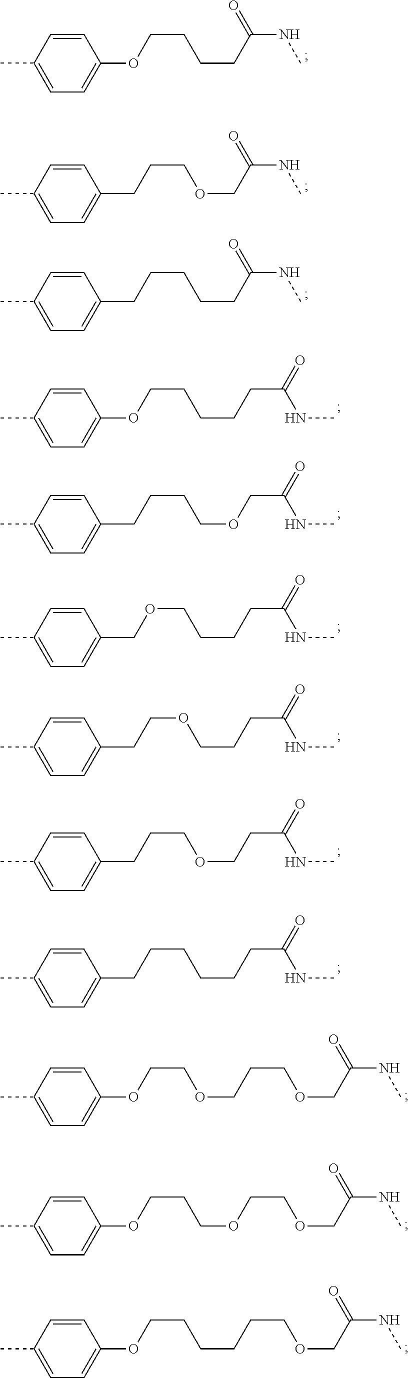

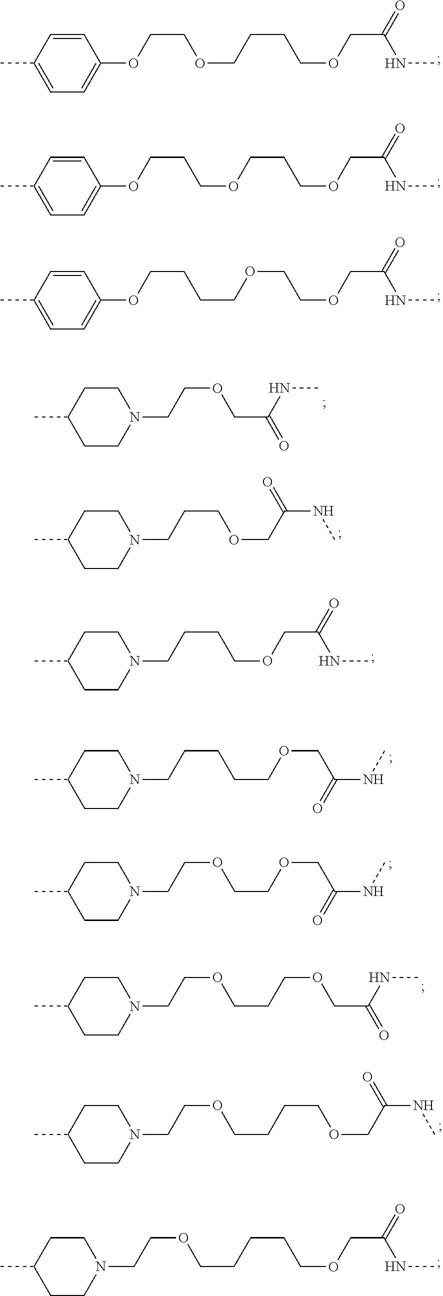

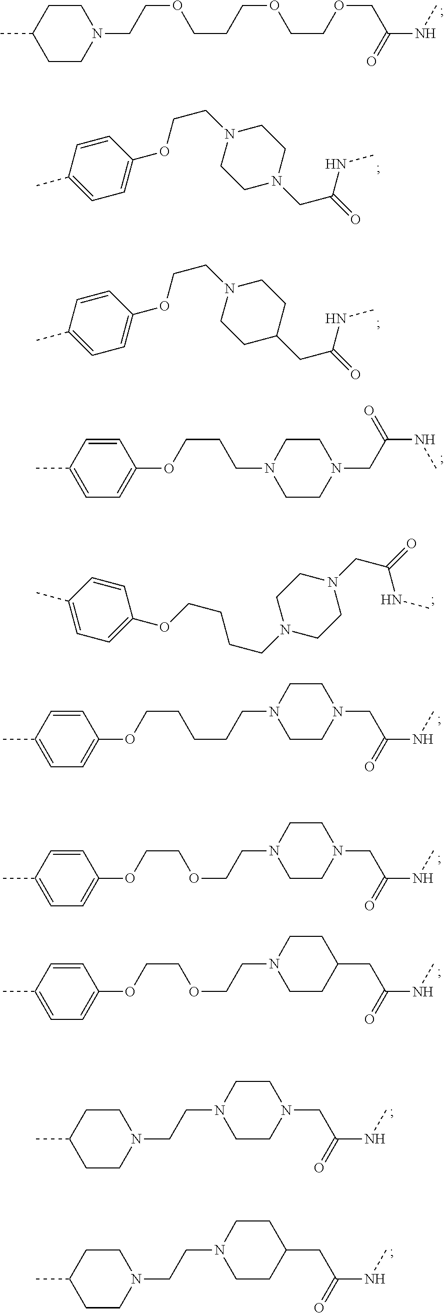

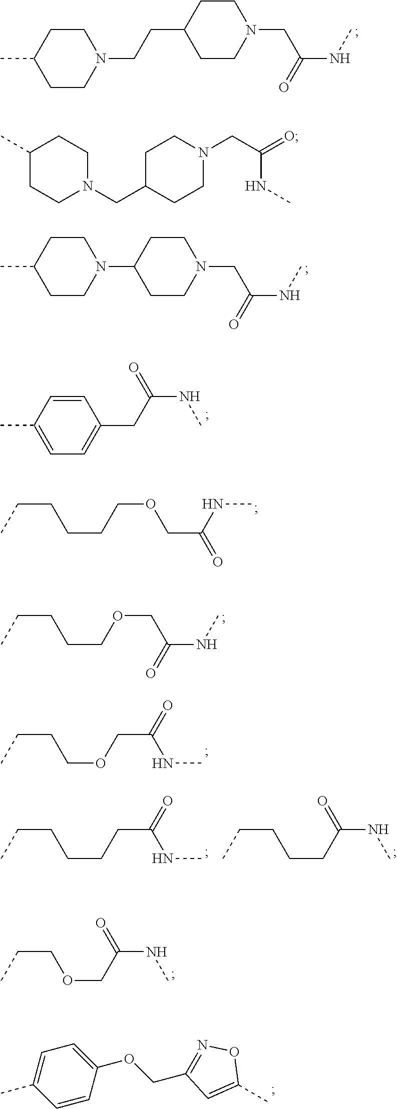

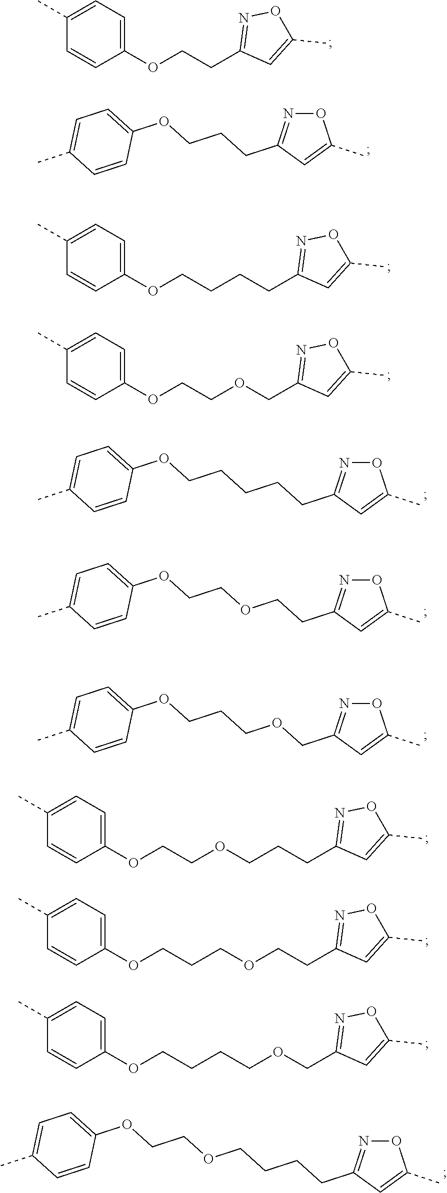

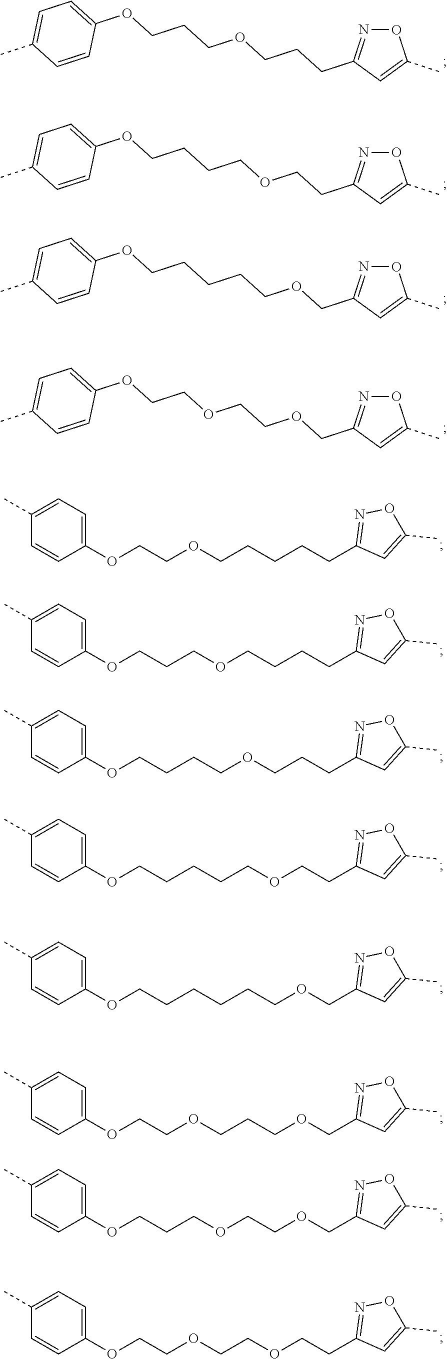

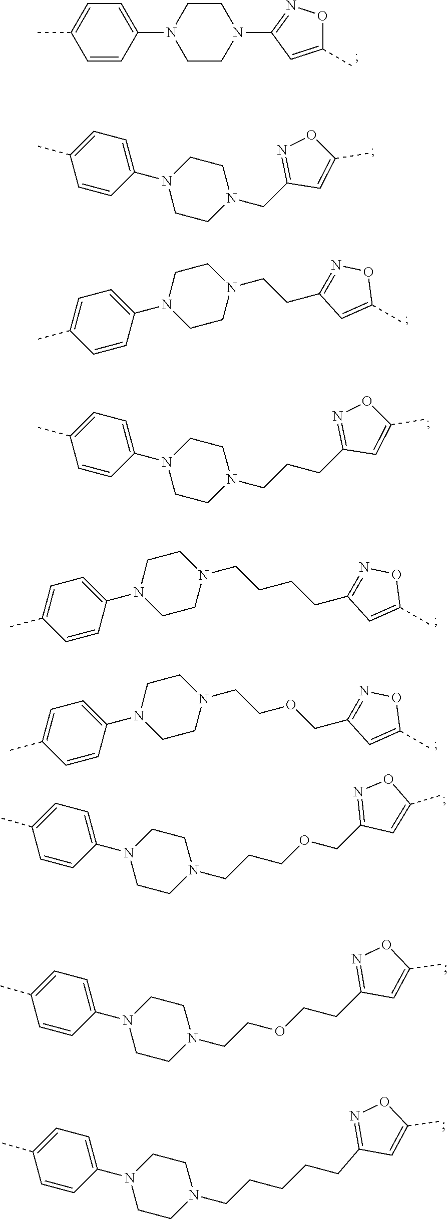

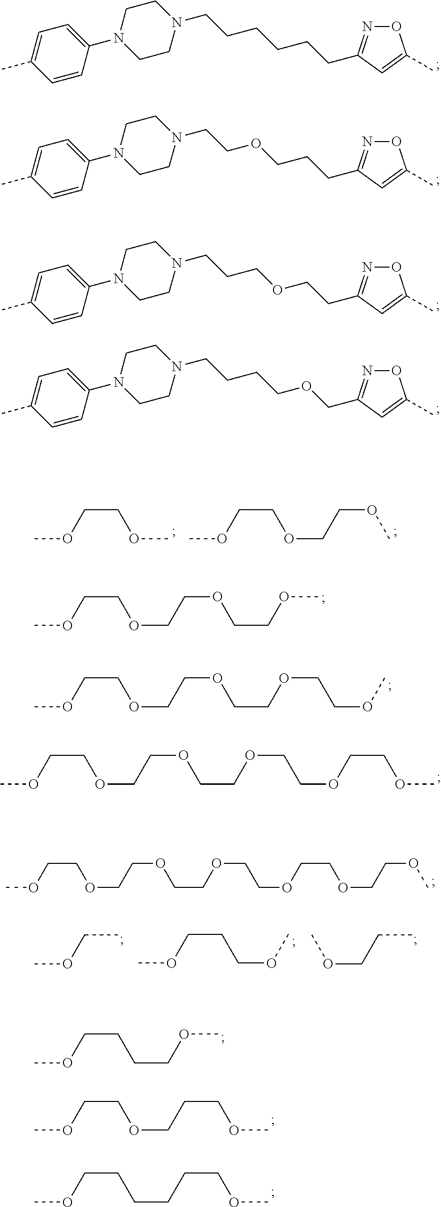

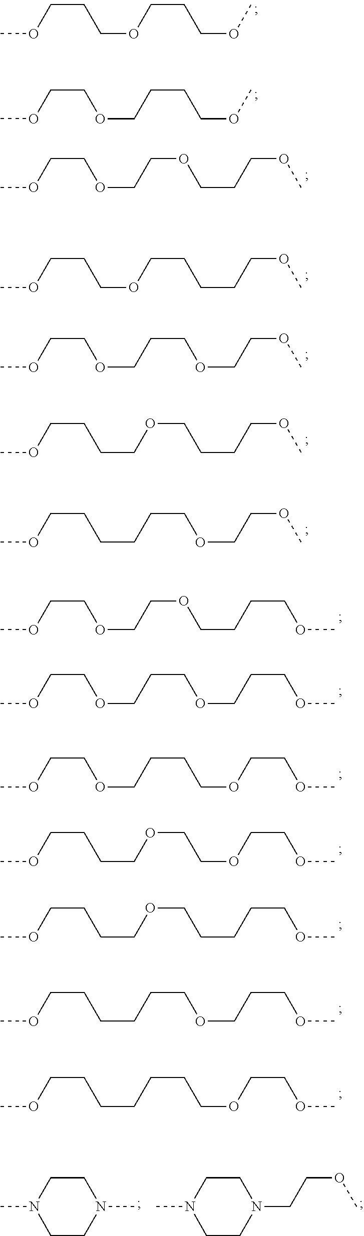

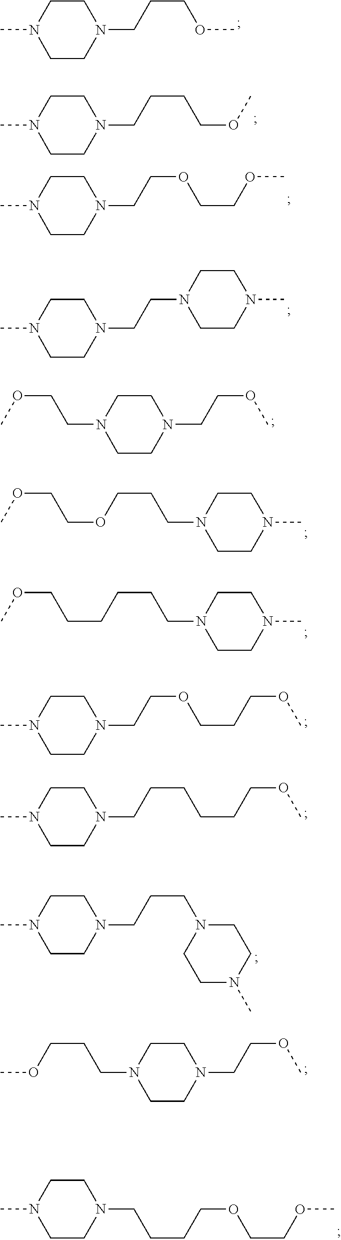

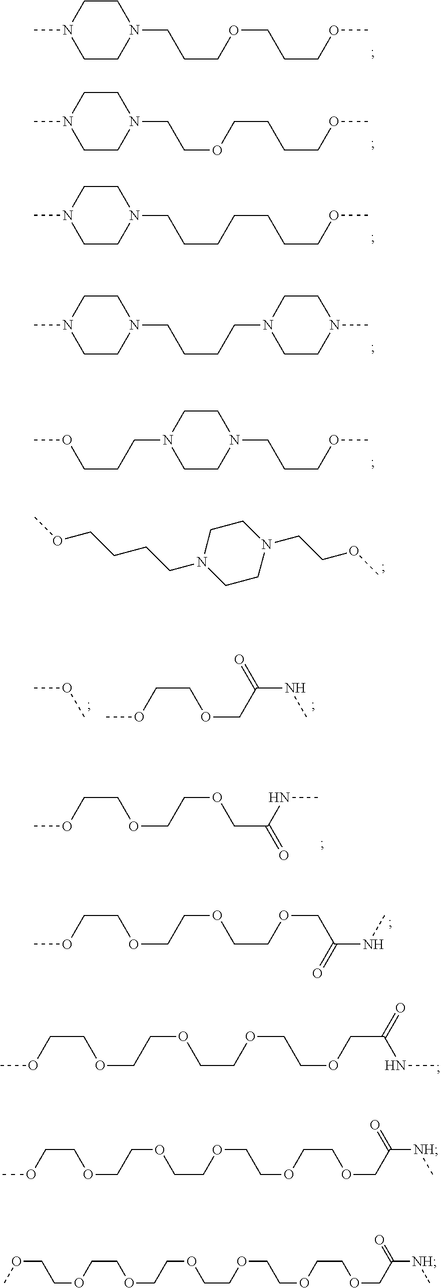

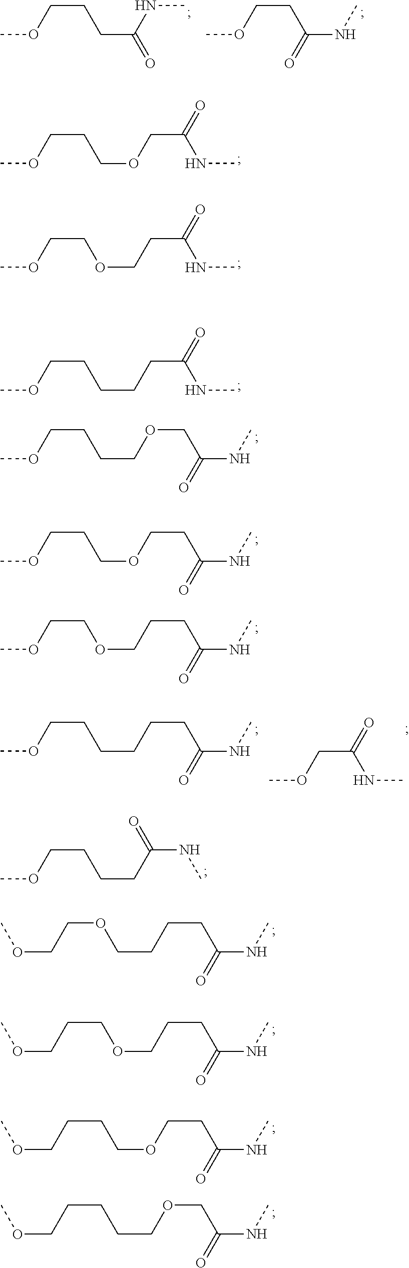

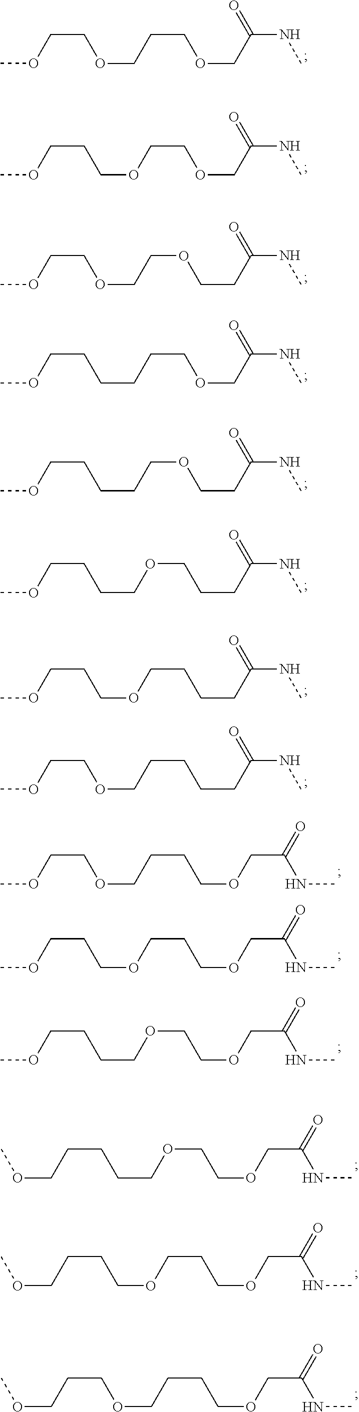

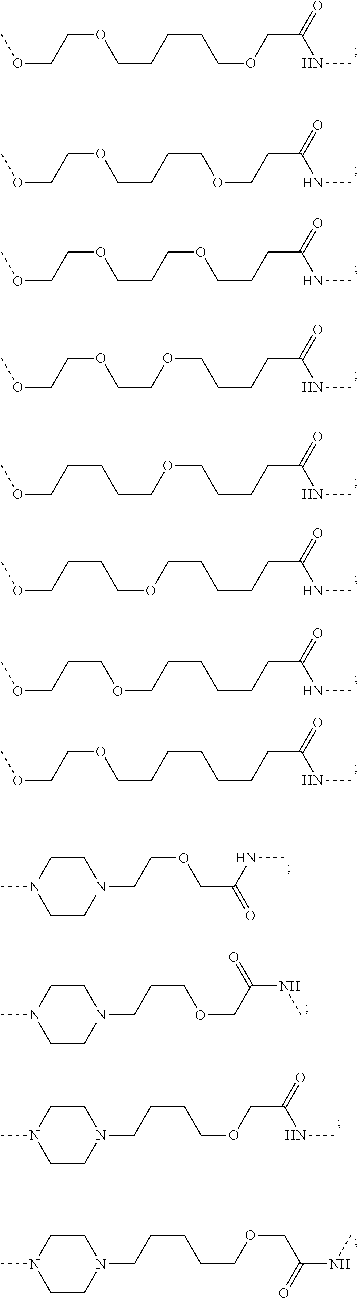

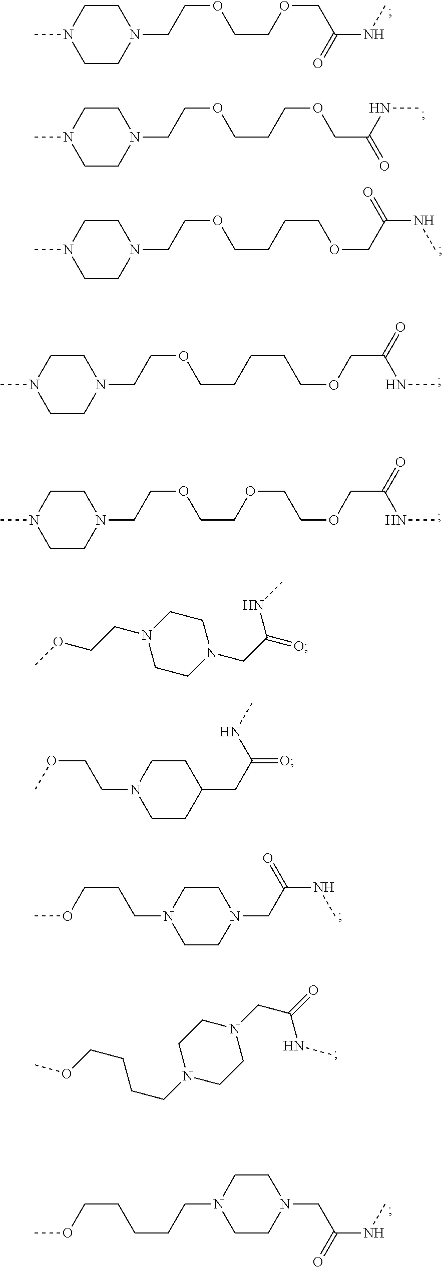

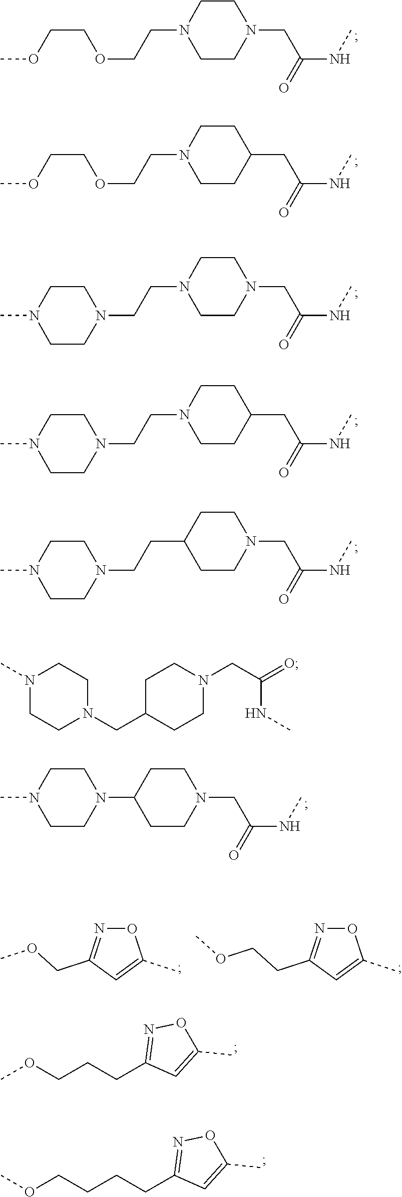

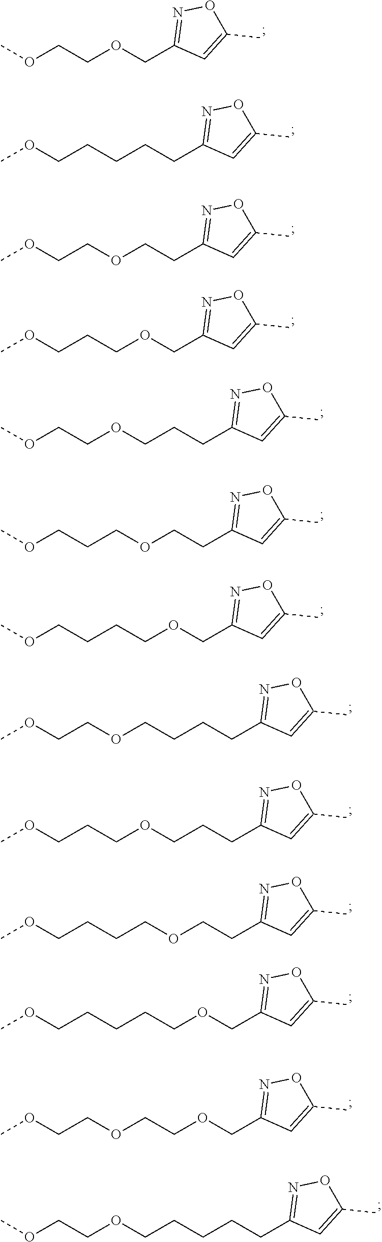

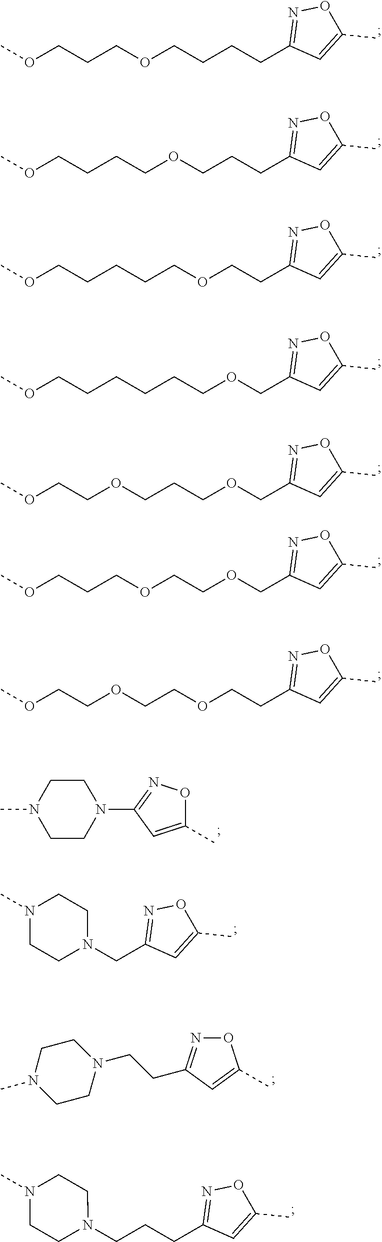

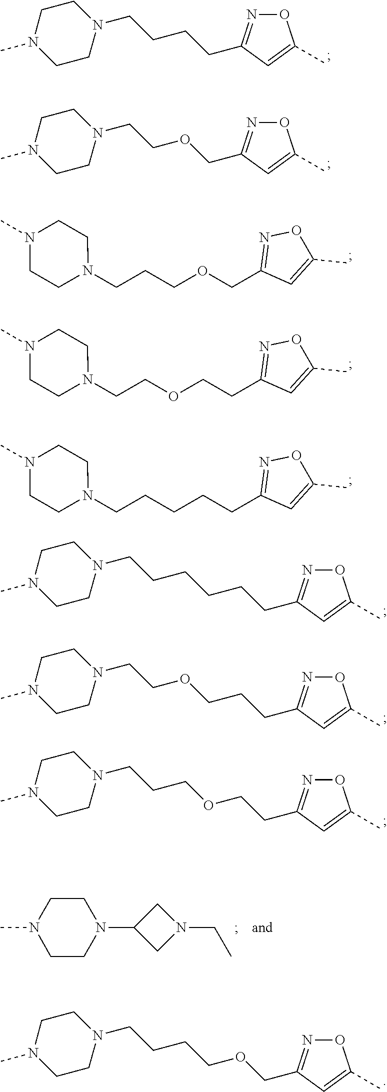

In additional embodiments, the linker (L) comprises a structure selected from, but not limited to the structure shown below, where a dashed line indicates the attachment point to the PTM or ULM moieties:

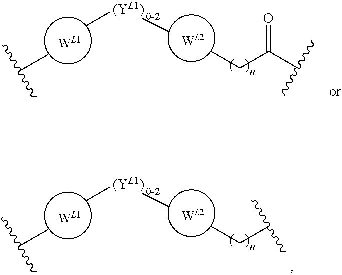

wherein.
W^{L1} and W¹² are each independently absent, a 4-8 membered ring with 0-4 heteroatoms, optionally substituted with R^{Q}, each R^{Q} is independently a H, halo, OH, CN, CF₃, optionally substituted linear or branched C₁-C₆ alkyl, optionally substituted linear or branched C₁-C₆ alkoxy, or 2 R^{Q} groups taken together with the atom they are attached to, form a 4-8 membered ring system containing 0-4 heteroatoms;
Y^{L1} is each independently a bond, optionally substituted linear or branched C₁-C₆ alkyl and optionally one or more C atoms are replaced with O; or optionally substituted linear or branched C₁-C₆ alkoxy;
n is 0-10; and

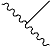

indicates the attachment point to the PTM or ULM moieties.

In additional embodiments, the linker (L) comprises a structure selected from, but not limited to the structure shown below, where a dashed line indicates the attachment point to the PTM or ULM moieties:

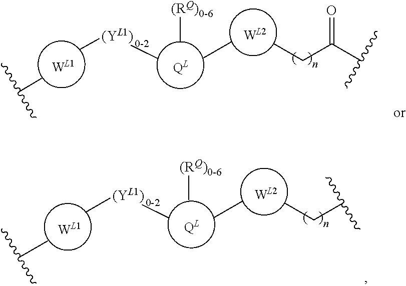

wherein:
W^{L1} and W¹² are each independently absent, aryl, heteroaryl, cyclic, heterocyclic, C₁₋₆ alkyl and optionally one or more C atoms are replaced with O, C₁₋₆ alkene and optionally one or more C atoms are replaced with O, C₁₋₆ alkyne and optionally one or more C atoms are replaced with O, bicyclic, biaryl, biheteroaryl, or biheterocyclic, each optionally substituted with R^{Q}, each R^{Q} is independently a H, halo, OH, CN, CF₃, hydroxyl, nitro, C ≡CH, C₂₋₆ alkenyl, C₂₋₆ alkynyl, optionally substituted linear or branched C₁-C₆ alkyl, optionally substituted linear or branched C₁-C₆ alkoxy, optionally substituted linear or branched OC₁₋₃alkyl (e.g., optionally substituted by 1 or more —F), OH, NH₂, NR^{Y1}R^{Y2}, CN, or 2 R^{Q} groups taken together with the atom they are attached to, form a 4-8 membered ring system containing 0-4 heteroatoms;
Y^{L1} is each independently a bond, NR^{YL1}, O, S, NR^{YL2}, CR^{YL1}R^{YL2}, C═O, C═S, SO, SO₂, optionally substituted linear or branched C₁-C₆ alkyl and optionally one or more C atoms are replaced with O; optionally substituted linear or branched C₁-C₆ alkoxy;
Q^{L} is a 3-6 membered alicyclic or aromatic ring with 0-4 heteroatoms, optionally bridged, optionally substituted with 0-6 R^{Q}, each R^{Q} is independently H, optionally substituted linear or branched C₁₋₆ alkyl (branched. g., optionally substituted by 1 or more halo or C₁₋₆ alkoxyl), or 2 R^{Q} groups taken together with the atom they are attached to, form a 3-8 membered ring system containing 0-2 heteroatoms;
R^{YL1}, R^{YL2} are each independently H, OH, optionally substituted linear or branched C₁₋₆ alkyl (branched. g., optionally substituted by 1 or more halo or C₁₋₆ alkoxyl), or R¹, R² together with the atom they are attached to, form a 3-8 membered ring system containing 0-2 heteroatoms;
n is 0-10; and

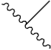

indicates the attachment point to the PTM or ULM moieties.

In additional embodiments, the linker group is optionally substituted (polyethyleneglycol having between 1 and about 100 ethylene glycol units (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, etc., ethylene glycol units), between about 1 and about 50 ethylene glycol units, between 1 and about 25 ethylene glycol units, between about 1 and 10 ethylene glycol units, between 1 and about 8 ethylene glycol units and 1 and 6 ethylene glycol units, between 2 and 4 ethylene glycol units, or optionally substituted alkyl groups interdispersed with optionally substituted, O, N, S, P or Si atoms. In certain embodiments, the linker is substituted with an aryl, phenyl, benzyl, alkyl, alkylene, or heterocycle group. In certain embodiments, the linker may be asymmetric or symmetrical.

In any of the embodiments of the compounds described herein, the linker group may be any suitable moiety as described herein. In one embodiment, the linker is a substituted or unsubstituted polyethylene glycol group ranging in size from about 1 to about 12 ethylene glycol units, between 1 and about 10 ethylene glycol units, about 2 about 6 ethylene glycol units, between about 2 and 5 ethylene glycol units, between about 2 and 4 ethylene glycol units.

In another embodiment, the present disclosure is directed to a compound which comprises a PTM group as described above, which binds to a target protein or polypeptide (e.g., PTK2/FAK Receptor), which is ubiquitinated by an ubiquitin ligase and is chemically linked directly to the ULM group or through a linker moiety L, or PTM is alternatively a ULM′ group which is also a ubiquitin ligase binding moiety, which may be the same or different than the ULM group as described above and is linked directly to the ULM group directly or through the linker moiety; and L is a linker moiety as described above which may be present or absent and which chemically (covalently) links ULM to PTM, or a pharmaceutically acceptable salt, enantiomer, stereoisomer, solvate or polymorph thereof.

In certain embodiments, the linker group L is a group comprising one or more covalently connected structural units independently selected from the group consisting of:

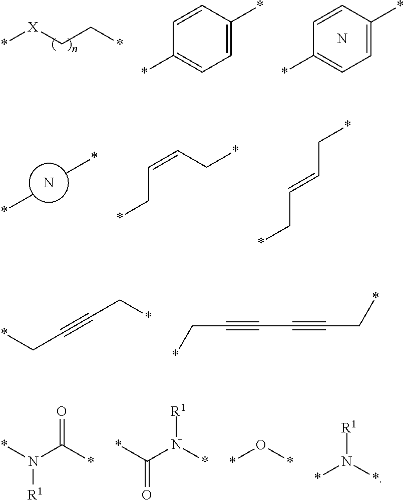

The X is selected from the group consisting of O, N, S, S(O) and SO₂; n is integer from 1 to 5; R^{L1} is hydrogen or alkyl,

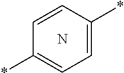

is a mono- or bicyclic aryl or heteroaryl optionally substituted with 1-3 substituents selected from alkyl, halogen, haloalkyl, hydroxy, alkoxy or cyano;

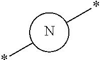

is a mono- or bicyclic cycloalkyl or a heterocycloalkyl optionally substituted with 1-3 substituents selected from alkyl, halogen, haloalkyl, hydroxy, alkoxy or cyano; and the phenyl ring fragment can be optionally substituted with 1, 2 or 3 substituents selected from the group consisting of alkyl, halogen, haloalkyl, hydroxy, alkoxy and cyano. In an embodiment, the linker group L comprises up to 10 covalently connected structural units, as described above.

Although the ULM group and PTM group may be covalently linked to the linker group through any group which is appropriate and stable to the chemistry of the linker, in preferred aspects of the present disclosure, the linker is independently covalently bonded to the ULM group and the PTM group preferably through an amide, ester, thioester, keto group, carbamate (urethane), carbon or ether, each of which groups may be inserted anywhere on the ULM group and PTM group to provide maximum binding of the ULM group on the ubiquitin ligase and the PTM group on the target protein to be degraded. (It is noted that in certain aspects where the PTM group is a ULM group, the target protein for degradation may be the ubiquitin ligase itself). In certain preferred aspects, the linker may be linked to an optionally substituted alkyl, alkylene, alkene or alkyne group, an aryl group or a heterocyclic group on the ULM and/or PTM groups.

### Exemplary PTMs

In preferred aspects of the disclosure, the PTM group is a group, which binds to target proteins. Targets of the PTM group are numerous in kind and are selected from proteins that are expressed in a cell such that at least a portion of the sequences is found in the cell and may bind to a PTM group. The term “protein” includes oligopeptides and polypeptide sequences of sufficient length that they can bind to a PTM group according to the present disclosure. Any protein in a eukaryotic system or a microbial system, including a virus, bacteria or fungus, as otherwise described herein, are targets for ubiquitination mediated by the compounds according to the present disclosure. Preferably, the target protein is a eukaryotic protein.

PTM groups according to the present disclosure include, for example, any moiety which binds to a protein specifically (binds to a target protein) and includes the following non-limiting examples of small molecule target protein moieties: focal adhesion kinase FAK/PTK2 inhibitors, Bruton's tyrosine kinase (BTK) inhibitors, KRas inhibitors, Hsp90 inhibitors, kinase inhibitors, HDM2 & MDM2 inhibitors, compounds targeting Human BET Bromodomain-containing proteins, HOAC inhibitors, human lysine methyltransferase inhibitors, angiogenesis inhibitors, nuclear hormone receptor compounds, immunosuppressive compounds, and compounds targeting the aryl hydrocarbon receptor (AHR), among numerous others. The compositions described below exemplify some of the members of small molecule target protein binding moieties. Such small molecule target protein binding moieties also include pharmaceutically acceptable salts, enantiomers, solvates and polymorphs of these compositions, as well as other small molecules that may target a protein of interest, such as B focal adhesion kinase FAK/protein-tyrosine kinase 2 (PTK2) and/or mutant FAKs/PTK2s, including gain-of-function AKs/PTK2s, mutant(s), including both wild-type and mutant forms, such as FAK-Del33. These binding moieties are linked to the ubiquitin ligase binding moiety preferably through a linker in order to present a target protein (to which the protein target moiety is bound) in proximity to the ubiquitin ligase for ubiquitination and degradation.

Any protein, which can bind to a protein target moiety or PTM group and acted on or degraded by an ubiquitin ligase is a target protein according to the present disclosure. In general, target proteins may include, for example, structural proteins, receptors, enzymes, cell surface proteins, proteins pertinent to the integrated function of a cell, including proteins involved in catalytic activity, aromatase activity, motor activity, helicase activity, metabolic processes (anabolism and catabolism), antioxidant activity, proteolysis, biosynthesis, proteins with kinase activity, oxidoreductase activity, transferase activity, hydrolase activity, lyase activity, isomerase activity, ligase activity, enzyme regulator activity, signal transducer activity, structural molecule activity, binding activity (protein, lipid carbohydrate), receptor activity, cell motility, membrane fusion, cell communication, regulation of biological processes, development, cell differentiation, response to stimulus, behavioral proteins, cell adhesion proteins, proteins involved in cell death, proteins involved in transport (including protein transporter activity, nuclear transport, ion transporter activity, channel transporter activity, carrier activity, permease activity, secretion activity, electron transporter activity, pathogenesis, chaperone regulator activity, nucleic acid binding activity, transcription regulator activity, extracellular organization and biogenesis activity, translation regulator activity. Proteins of interest can include proteins from eukaryotes and prokaryotes including humans as targets for drug therapy, other animals, including domesticated animals, microbials for the determination of targets for antibiotics and other antimicrobials and plants, and even viruses, among numerous others.

The present disclosure may be used to treat a number of disease states and/or conditions, including any disease state and/or condition in which proteins are dysregulated and where a patient would benefit from the degradation and/or inhibition of proteins.

In an additional aspect, the description provides therapeutic compositions comprising an effective amount of a compound as described herein or salt form thereof, and a pharmaceutically acceptable carrier, additive or excipient, and optionally an additional bioactive agent. The therapeutic compositions modulate protein degradation in a patient or subject, for example, an animal such as a human, and can be used for treating or ameliorating disease states or conditions which are modulated through the degraded protein. In certain embodiments, the therapeutic compositions as described herein may be used to effectuate the degradation of proteins of interest for the treatment or amelioration of a disease, e.g., cancer, (such as, solid tumors, carcinoma, adenocarcinoma, cystadenocarcinoma, endometriod carcinoma, mesothelioma, sarcoma, breast cancer, ovarian cancer, lung cancer, head and neck cancer, colorectal cancer, bladder cancer, uterine cancer, prostate cancer, squamous cell carcinoma, leukemia, glioblastoma and renal cancer.). In certain particular embodiments, the disease is ovarian cancer. In other particular embodiments, the disease is breast cancer.

In alternative aspects, the present disclosure relates to a method for treating a disease state or ameliorating the symptoms of a disease or condition in a subject in need thereof by degrading a protein or polypeptide through which a disease state or condition is modulated comprising administering to said patient or subject an effective amount, e.g., a therapeutically effective amount, of at least one compound as described hereinabove, optionally in combination with a pharmaceutically acceptable carrier, additive or excipient, and optionally an additional bioactive agent, wherein the composition is effective for treating or ameliorating the disease or disorder or symptom thereof in the subject. The method according to the present disclosure may be used to treat a large number of disease states or conditions including cancer, (such as, solid tumors, carcinoma, adenocarcinoma, cystadenocarcinoma, endometriod carcinoma, mesothelioma, sarcoma, breast cancer, ovarian cancer, lung cancer, head and neck cancer, colorectal cancer, bladder cancer, uterine cancer, prostate cancer, squamous cell carcinoma, leukemia, glioblastoma and renal cancer), by virtue of the administration of effective amounts of at least one compound described herein. The disease state or condition may be a disease caused by a microbial agent or other exogenous agent such as a virus, bacteria, fungus, protozoa or other microbe or may be a disease state, which is caused by overexpression of a protein, which leads to a disease state and/or condition.

In another aspect, the description provides methods for identifying the effects of the degradation of proteins of interest in a biological system using compounds according to the present disclosure.

The term “target protein” is used to describe a protein or polypeptide, which is a target for binding to a compound according to the present disclosure and degradation by ubiquitin ligase hereunder. For example, in any aspect or embodiment described herein, the PTM is a small molecule comprising a focal adhesion kinase (FAK or PTH2) targeting moiety. Such small molecule target protein binding moieties also include pharmaceutically acceptable salts, enantiomers, solvates and polymorphs of these compositions, as well as other small molecules that may target a protein of interest. These binding moieties are linked to at least one ULM group (e.g. VLM, CLM, ILM, and/or MLM) through at least one linker group L.

Target proteins, which may be bound to the protein target moiety and degraded by the ligase to which the ubiquitin ligase binding moiety is bound, include any protein or peptide, including fragments thereof, analogues thereof, and/or homologues thereof. Target proteins include proteins and peptides having any biological function or activity including structural, regulatory, hormonal, enzymatic, genetic, immunological, contractile, storage, transportation, and signal transduction. More specifically, a number of drug targets for human therapeutics represent protein targets to which protein target moiety may be bound and incorporated into compounds according to the present disclosure. These include proteins which may be used to restore function in numerous polygenic diseases, including for example FAK, PTK2, FAKdel33, B7.1 and B7, TINFRlm, TNFR2, NADPH oxidase, BclIBax and other partners in the apotosis pathway, C5a receptor, HMG-CoA reductase, PDE V phosphodiesterase type, PDE IV phosphodiesterase type 4, PDE I, PDEII, PDEIII, squalene cyclase inhibitor, CXCR1, CXCR2, nitric oxide (NO) synthase, cyclo-oxygenase 1, cyclo-oxygenase 2, 5HT receptors, dopamine receptors, G Proteins, i.e., Gq, histamine receptors, 5-lipoxygenase, tryptase serine protease, thymidylate synthase, purine nucleoside phosphorylase, GAPDH trypanosomal, glycogen phosphorylase, Carbonic anhydrase, chemokine receptors, JAW STAT, RXR and similar, HIV 1 protease, HIV 1 integrase, influenza, neuramimidase, hepatitis B reverse transcriptase, sodium channel, multi drug resistance (MDR), protein P-glycoprotein (and MRP), tyrosine kinases, CD23, CD124, tyrosine kinase p56 lck, CD4, CD5, IL-2 receptor, IL-1 receptor, TNF-alphaR, ICAM1, Cat+ channels, VCAM, VLA-4 integrin, selectins, CD40/CD40L, newokinins and receptors, inosine monophosphate dehydrogenase, p38 MAP Kinase, Ras1Raf1MEWERK pathway, interleukin-1 converting enzyme, caspase, HCV, NS3 protease, HCV NS3 RNA helicase, glycinamide ribonucleotide formyl transferase, rhinovirus 3C protease, herpes simplex virus-1 (HSV-I), protease, cytomegalovirus (CMV) protease, poly (ADP-ribose) polymerase, cyclin dependent kinases, vascular endothelial growth factor, oxytocin receptor, microsomal transfer protein inhibitor, bile acid transport inhibitor, 5 alpha reductase inhibitors, angiotensin 11, glycine receptor, noradrenaline reuptake receptor, endothelin receptors, neuropeptide Y and receptor, estrogen receptors, androgen receptors, adenosine receptors, adenosine kinase and AMP deaminase, purinergic receptors (P2Y1, P2Y2, P2Y4, P2Y6, P2X1-7), famesyltransferases, geranylgeranyl transferase, TrkA a receptor for NGF, beta-amyloid, tyrosine kinase Flk-HKDR, vitronectin receptor, integrin receptor, Her-21 neu, telomerase inhibition, cytosolic phospholipaseA2 and EOF receptor tyrosine kinase. Additional protein targets include, for example, ecdysone 20-monooxygenase, ion channel of the GABA gated chloride channel, acetylcholinesterase, voltage-sensitive sodium channel protein, calcium release channel, and chloride channels. Still further target proteins include Acetyl-CoA carboxylase, adenylosuccinate synthetase, protoporphyrinogen oxidase, and enolpyruvylshikimate-phosphate synthase.

These various protein targets may be used in screens that identify compound moieties which bind to the protein and by incorporation of the moiety into compounds according to the present disclosure, the level of activity of the protein may be altered for therapeutic end result.

The term “protein target moiety” or PTM is used to describe a small molecule which binds to a target protein or other protein or polypeptide of interest and places/presents that protein or polypeptide in proximity to an ubiquitin ligase such that degradation of the protein or polypeptide by ubiquitin ligase may occur. Non-limiting examples of small molecule target protein binding moieties include focal adhesion kinase (FAK/PK2) inhibitors, Bruton's tyrosine kinase (BTK) inhibitors, KRas inhibitors, Hsp90 inhibitors, kinase inhibitors, MDM2 inhibitors, compounds targeting Human BET Bromodomain-containing proteins, HOAC inhibitors, human lysine methyltransferase inhibitors, angiogenesis inhibitors, immunosuppressive compounds, and compounds targeting the aryl hydrocarbon receptor (AHR), among numerous others. The compositions described below exemplify some of the members of the small molecule target proteins.

Exemplary protein target moieties according to the present disclosure include, focal adhesion kinase (FAK/PK2) inhibitors, Bruton's tyrosine kinase (BTK) inhibitors, KRas inhibitors, haloalkane halogenase inhibitors, Hsp90 inhibitors, kinase inhibitors, MDM2 inhibitors, compounds targeting Human BET Bromodomain-containing proteins, HOAC inhibitors, human lysine methyltransferase inhibitors, angiogenesis inhibitors, immunosuppressive compounds, and compounds targeting the aryl hydrocarbon receptor (AHR).

The compositions described herein exemplify some of the members of these types of small molecule target protein binding moieties. Such small molecule target protein binding moieties also include pharmaceutically acceptable salts, enantiomers, solvates and polymorphs of these compositions, as well as other small molecules that may target a protein of interest. References which are cited herein below are incorporated by reference herein in their entirety.

In any aspect or embodiment described herein, the PTM is a Bruton's tyrosine kinase (BTK) binding/targeting moiety, e.g., a small molecule comprising a Bruton's tyrosine kinase (BTK) binding/targeting moiety. In any aspect or embodiment described herein, the PTM binds mutant Bruton's tyrosine kinase (BTK), e.g. gain-of-function mutant BTKs. In a particular aspect or embodiment described herein, the PTM has a chemical structure represented by:

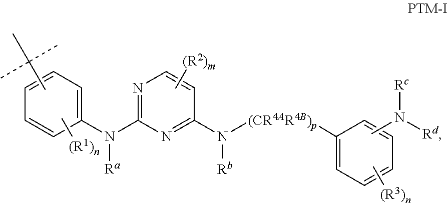

wherein:
each of R¹, R², R³, R^{4A}, R^{4B} is independently hydrogen, halogen, C₁-C₈ alkyl, C₁-C₈ haloalkyl, C₂-C₈ alkenyl, C₂-C₈ alkynyl, C₁-C₈ alkloxy, C₃-C₈ cycloalkyl, heterocycloalkyl, aryl, heteroaryl, primary amino, secondary amino, amido, carboxyl, acetyl or cyano;
each of R^{a}, R^{b}, R^{c}, and R^{d} is independently hydrogen, C₁-C₈ alkyl, C₁-C₈ haloalkyl, C₂-C₈ alkenyl, C₂-C₈ alkynyl, C₃-C₈ cycloalkyl, heterocycloalkyl, aryl, heteroaryl, or SO2-R^{e};
each R^{e} is independently C₁-C₄ alkyl;
each n is independently an integer from 0 to 4;
m is an integer from 0 to 2;
p is an integer from 1 to 8; and
the

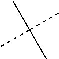

indicates the site of attachment of at least one of a linker, ULM, ULM′, CLM, CLM′, VLM, VLM′, ILM, ILM′, MLM, MLM′, or a combination thereof.

In another particular aspect or embodiment described herein, the PTM has a chemical structure represented by:

wherein:
R² is hydrogen, halogen, C₁-C₄ alkyl, or C₁-C₄ haloalkyl;
R³ is hydrogen, halogen, C₁-C₄ alkyl, C₁-C₄ alkoxy, or —CN;
each of R^{a}, R^{b}, and R^{c} are independently, hydrogen or C₁-C₄ alkyl;
R^{e} is C₁-C₁ alkyl; and
the

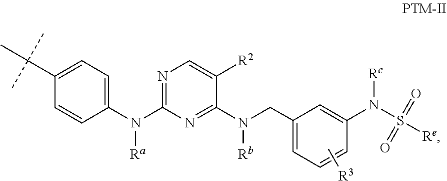

the

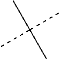

indicates the site of attachment of at least one of a linker, ULM, ULM′, CLM, CLM′, VLM, VLM′, ILM, ILM′, MLM, MLM′, or a combination thereof.

In any aspect or embodiment described herein, the PTM is

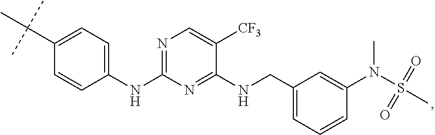

wherein the

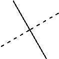

indicates the site of attachment of at least one of a linker, ULM, ULM′, CLM, CLM′, VLM, VLM′, ILM, ILM′, MLM, MLM′, or a combination thereof.

### Therapeutic Compositions

Pharmaceutical compositions comprising combinations of an effective amount of at least one bifunctional compound as described herein, and one or more of the compounds otherwise described herein, all in effective amounts, in combination with a pharmaceutically effective amount of a carrier, additive or excipient, represents a further aspect of the present disclosure.

The present disclosure includes, where applicable, the compositions comprising the pharmaceutically acceptable salts, in particular, acid or base addition salts of compounds as described herein. The acids which are used to prepare the pharmaceutically acceptable acid addition salts of the aforementioned base compounds useful according to this aspect are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3 naphthoate)] salts, among numerous others.

Pharmaceutically acceptable base addition salts may also be used to produce pharmaceutically acceptable salt forms of the compounds or derivatives according to the present disclosure. The chemical bases that may be used as reagents to prepare pharmaceutically acceptable base salts of the present compounds that are acidic in nature are those that form non-toxic base salts with such compounds. Such non-toxic base salts include, but are not limited to those derived from such pharmacologically acceptable cations such as alkali metal cations (eg., potassium and sodium) and alkaline earth metal cations (eg, calcium, zinc and magnesium), ammonium or water-soluble amine addition salts such as N-methylglucamine-(meglumine), and the lower alkanolammonium and other base salts of pharmaceutically acceptable organic amines, among others.

The compounds as described herein may, in accordance with the disclosure, be administered in single or divided doses by the oral, parenteral or topical routes. Administration of the active compound may range from continuous (intravenous drip) to several oral administrations per day (for example, Q.I.D.) and may include oral, topical, parenteral, intramuscular, intravenous, sub-cutaneous, transdermal (which may include a penetration enhancement agent), buccal, sublingual and suppository administration, among other routes of administration. Enteric coated oral tablets may also be used to enhance bioavailability of the compounds from an oral route of administration. The most effective dosage form will depend upon the pharmacokinetics of the particular agent chosen as well as the severity of disease in the patient. Administration of compounds according to the present disclosure as sprays, mists, or aerosols for intra-nasal, intra-tracheal or pulmonary administration may also be used. The present disclosure therefore also is directed to pharmaceutical compositions comprising an effective amount of compound as described herein, optionally in combination with a pharmaceutically acceptable carrier, additive or excipient. Compounds according to the present disclosure may be administered in immediate release, intermediate release or sustained or controlled release forms. Sustained or controlled release forms are preferably administered orally, but also in suppository and transdermal or other topical forms. Intramuscular injections in liposomal form may also be used to control or sustain the release of compound at an injection site.

The compositions as described herein may be formulated in a conventional manner using one or more pharmaceutically acceptable carriers and may also be administered in controlled-release formulations. Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions as described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions as described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Helv or similar alcohol.

The pharmaceutical compositions as described herein may be orally administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, the pharmaceutical compositions as described herein may be administered in the form of suppositories for rectal administration. These can be prepared by mixing the agent with a suitable non-irritating excipient, which is solid at room temperature but liquid at rectal temperature and therefore will melt in the rectum to release the drug. Such materials include cocoa butter, beeswax and polyethylene glycols.

The pharmaceutical compositions as described herein may also be administered topically. Suitable topical formulations are readily prepared for each of these areas or organs. Topical application for the lower intestinal tract can be effected in a rectal suppository formulation (see above) or in a suitable enema formulation. Topically-acceptable transdermal patches may also be used.

For topical applications, the pharmaceutical compositions may be formulated in a suitable ointment containing the active component suspended or dissolved in one or more carriers. Carriers for topical administration of the compounds of this disclosure include, but are not limited to, mineral oil, liquid petrolatum, white petrolatum, propylene glycol, polyoxyethylene, polyoxypropylene compound, emulsifying wax and water. In certain preferred aspects of the disclosure, the compounds may be coated onto a stent which is to be surgically implanted into a patient in order to inhibit or reduce the likelihood of occlusion occurring in the stent in the patient.

Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with our without a preservative such as benzylalkonium chloride. Alternatively, for ophthalmic uses, the pharmaceutical compositions may be formulated in an ointment such as petrolatum.

The pharmaceutical compositions as described herein may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of compound in a pharmaceutical composition as described herein that may be combined with the carrier materials to produce a single dosage form will vary depending upon the host and disease treated, the particular mode of administration. Preferably, the compositions should be formulated to contain between about 0.05 milligram to about 750 milligrams or more, more preferably about 1 milligram to about 600 milligrams, and even more preferably about 10 milligrams to about 500 milligrams of active ingredient, alone or in combination with at least one other compound according to the present disclosure.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

A patient or subject in need of therapy using compounds according to the methods described herein can be treated by administering to the patient (subject) an effective amount of the compound according to the present disclosure including pharmaceutically acceptable salts, solvates or polymorphs, thereof optionally in a pharmaceutically acceptable carrier or diluent, either alone, or in combination with other known therapeutic agents as otherwise identified herein.

These compounds can be administered by any appropriate route, for example, orally, parenterally, intravenously, intradermally, subcutaneously, or topically, including transdermally, in liquid, cream, gel, or solid form, or by aerosol form.

The active compound is included in the pharmaceutically acceptable carrier or diluent in an amount sufficient to deliver to a patient a therapeutically effective amount for the desired indication, without causing serious toxic effects in the patient treated. A preferred dose of the active compound for all of the herein-mentioned conditions is in the range from about 10 ng/kg to 300 mg/kg, preferably 0.1 to 100 mg/kg per day, more generally 0.5 to about 25 mg per kilogram body weight of the recipient/patient per day. A typical topical dosage will range from 0.01-5% wt/wt in a suitable carrier.

The compound is conveniently administered in any suitable unit dosage form, including but not limited to one containing less than 1 mg, 1 mg to 3000 mg, preferably 5 to 500 mg of active ingredient per unit dosage form. An oral dosage of about 25-250 mg is often convenient.

The active ingredient is preferably administered to achieve peak plasma concentrations of the active compound of about 0.00001-30 mM, preferably about 0.1-30 μM. This may be achieved, for example, by the intravenous injection of a solution or formulation of the active ingredient, optionally in saline, or an aqueous medium or administered as a bolus of the active ingredient. Oral administration is also appropriate to generate effective plasma concentrations of active agent.

The concentration of active compound in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

Oral compositions will generally include an inert diluent or an edible carrier. They may be enclosed in gelatin capsules or compressed into tablets. For the purpose of oral therapeutic administration, the active compound or its prodrug derivative can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or enteric agents.

The active compound or pharmaceutically acceptable salt thereof can be administered as a component of an elixir, suspension, syrup, wafer, chewing gum or the like. A syrup may contain, in addition to the active compounds, sucrose as a sweetening agent and certain preservatives, dyes and colorings and flavors.

The active compound or pharmaceutically acceptable salts thereof can also be mixed with other active materials that do not impair the desired action, or with materials that supplement the desired action, such as anti-cancer agents, including EPO and darbapoietin alfa, among others. In certain preferred aspects of the disclosure, one or more compounds according to the present disclosure are coadministered with another bioactive agent, such as an anti-cancer agent or a would healing agent, including an antibiotic, as otherwise described herein.

Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

Liposomal suspensions may also be pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound are then introduced into the container. The container is then swirled by hand to free lipid material from the sides of the container and to disperse lipid aggregates, thereby forming the liposomal suspension.

### Therapeutic Methods

In an additional aspect, the description provides therapeutic compositions comprising an effective amount of a compound as described herein or salt form thereof, and a pharmaceutically acceptable carrier. The therapeutic compositions modulate protein degradation in a patient or subject, for example, an animal such as a human, and can be used for treating or ameliorating disease states or conditions which are modulated through the degraded protein.

The terms “treat”, “treating”, and “treatment”, etc., as used herein, refer to any action providing a benefit to a patient for which the present compounds may be administered, including the treatment of any disease state or condition which is modulated through the protein to which the present compounds bind. Disease states or conditions, including cancer, such as, solid tumors, carcinoma, adenocarcinoma, cystadenocarcinoma, endometriod carcinoma, mesothelioma, sarcoma, breast cancer, ovarian cancer, lung cancer, head and neck cancer, colorectal cancer, bladder cancer, uterine cancer, prostate cancer, squamous cell carcinoma, leukemia, glioblastoma and renal cancer), which may be treated using compounds according to the present disclosure are set forth hereinabove.

The description provides therapeutic compositions as described herein for effectuating the degradation of proteins of interest for the treatment or amelioration of a disease, e.g. cancer (such as, solid tumors, carcinoma, adenocarcinoma, cystadenocarcinoma, endometriod carcinoma, mesothelioma, sarcoma, breast cancer, ovarian cancer, lung cancer, head and neck cancer, colorectal cancer, bladder cancer, uterine cancer, prostate cancer, squamous cell carcinoma, leukemia, glioblastoma and renal cancer.). In certain additional embodiments, the disease is multiple myeloma. As such, in another aspect, the description provides a method of ubiquitinating/degrading a target protein in a cell. In certain embodiments, the method comprises administering a bifunctional compound as described herein comprising, e.g., a ULM and a PTM, preferably linked through a linker moiety, as otherwise described herein, wherein the ULM is coupled to the PTM and wherein the ULM recognizes a ubiquitin pathway protein (e.g., an ubiquitin ligase, such as an E3 ubiquitin ligase including cereblon, VHL, IAP, and/or MDM2) and the PTM recognizes the target protein such that degradation of the target protein will occur when the target protein is placed in proximity to the ubiquitin ligase, thus resulting in degradation/inhibition of the effects of the target protein and the control of protein levels. The control of protein levels afforded by the present disclosure provides treatment of a disease state or condition, which is modulated through the target protein by lowering the level of that protein in the cell, e.g., cell of a patient. In certain embodiments, the method comprises administering an effective amount of a compound as described herein, optionally including a pharmaceutically acceptable excipient, carrier, adjuvant, another bioactive agent or combination thereof.

In additional embodiments, the description provides methods for treating or ameliorating a disease, disorder or symptom thereof in a subject or a patient, e.g., an animal such as a human, comprising administering to a subject in need thereof a composition comprising an effective amount, e.g., a therapeutically effective amount, of a compound as described herein or salt form thereof, and a pharmaceutically acceptable excipient, carrier, adjuvant, another bioactive agent or combination thereof, wherein the composition is effective for treating or ameliorating the disease or disorder or symptom thereof in the subject.

In another aspect, the description provides methods for identifying the effects of the degradation of proteins of interest in a biological system using compounds according to the present disclosure.

In another embodiment, the present disclosure is directed to a method of treating a human patient in need for a disease state or condition modulated through a protein where the degradation of that protein will produce a therapeutic effect in the patient, the method comprising administering to a patient in need an effective amount of a compound according to the present disclosure, optionally in combination with another bioactive agent. The disease state or condition may be a disease caused by a microbial agent or other exogenous agent such as a virus, bacteria, fungus, protozoa or other microbe or may be a disease state, which is caused by overexpression of a protein, which leads to a disease state and/or condition

The term “disease state or condition” is used to describe any disease state or condition wherein protein dysregulation (i.e., the amount of protein expressed in a patient is elevated) occurs and where degradation of one or more proteins in a patient may provide beneficial therapy or relief of symptoms to a patient in need thereof. In certain instances, the disease state or condition may be cured.

Disease states or conditions which may be treated using compounds according to the present disclosure include, for example, asthma, autoimmune diseases such as multiple sclerosis, various cancers, ciliopathies, cleft palate, diabetes, heart disease, hypertension, inflammatory bowel disease, mental retardation, mood disorder, obesity, refractive error, infertility, Angelman syndrome, Canavan disease, Coeliac disease, Charcot-Marie-Tooth disease, Cystic fibrosis, Duchenne muscular dystrophy, Haemochromatosis, Haemophilia, Klinefelter's syndrome, Neurofibromatosis, Phenylketonuria, Polycystic kidney disease, (PKD1) or 4 (PKD2) Prader-Willi syndrome, Sickle-cell disease, Tay-Sachs disease, Turner syndrome.

The term “neoplasia” or “cancer” is used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated. As used herein, the term neoplasia is used to describe all cancerous disease states and embraces or encompasses the pathological process associated with malignant hematogenous, ascitic and solid tumors. Exemplary cancers which may be treated by the present compounds either alone or in combination with at least one additional anti-cancer agent include squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Additional cancers which may be treated using compounds according to the present disclosure include, for example, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL and Philadelphia chromosome positive CML.

The term “bioactive agent” is used to describe an agent, other than a compound according to the present disclosure, which is used in combination with the present compounds as an agent with biological activity to assist in effecting an intended therapy, inhibition and/or prevention/prophylaxis for which the present compounds are used. Preferred bioactive agents for use herein include those agents which have pharmacological activity similar to that for which the present compounds are used or administered and include for example, anti-cancer agents, antiviral agents, especially including anti-HIV agents and anti-HCV agents, antimicrobial agents, antifungal agents, etc.

The term “additional anti-cancer agent” is used to describe an anti-cancer agent, which may be combined with compounds according to the present disclosure to treat cancer. These agents include, for example, everolimus, trabectedin, abraxane, TLK 286, AV-299, DN-101, pazopanib, GSK690693, RTA 744, ON 0910.Na, AZO 6244 (ARRY-142886), AMN-107, TKI-258, GSK461364, AZO 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HOAC inhibitor, a c-MET inhibitor, a PARP inhibitor, a Cdk inhibitor, an EGFR TK inhibitor, an IGFR-TK inhibitor, an anti-HGF antibody, a PD kinase inhibitor, an AKT inhibitor, an mTORC1/2 inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrozole, exemestane, letrozole, DES(diethylstilbestrol), estradiol, estrogen, conjugated estrogen, bevacizumab, IMC-1C₁₁, CHIR-258); 3-[5-(methylsulfonylpiperadinemethyl)-indolyl-quinolone, vatalanib, AG-013736, AVE-0005, goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafamib, BMS-214662, tipifamib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, adriamycin, bleomycin, buserelin, busulfan, carboplatin, carmustine, chlorambucil, cisplatin, cladribine, clodronate, cyproterone, cytarabine, dacarbazine, dactinomycin, daunombicin, diethylstilbestrol, epirubicin, fludarabine, fludrocortisone, fluoxymesterone, flutamide, gleevec, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, Vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunombicin, Edwina-asparaginase, strontium 89, casopitant, netupitant, an NK-1 receptor antagonist, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfllgrastim, erythropoietin, epoetin alfa, darbepoetin alfa and mixtures thereof.

The term “anti-HIV agent” or “additional anti-HIV agent” includes, for example, nucleoside reverse transcriptase inhibitors (NRTI), other non-nucloeoside reverse transcriptase inhibitors (i.e., those which are not representative of the present disclosure), protease inhibitors, fusion inhibitors, among others, exemplary compounds of which may include, for example, 3TC (Lamivudine), AZT (Zidovudine), (−)-FTC, ddI (Didanosine), ddC (zalcitabine), abacavir (ABC), tenofovir (PMPA), D-D4FC (Reverset), D4T (Stavudine), Racivir, L-FddC, L-FD4C, NVP (Nevirapine), DLV (Delavirdine), EFV (Efavirenz), SQVM (Saquinavir mesylate), RTV (Ritonavir), IDV (Indinavir), SQV (Saquinavir), NFV (Nelfinavir), APV (Amprenavir), LPV (Lopinavir), fusion inhibitors such as T20, among others, fuseon and mixtures thereof, including anti-HIV compounds presently in clinical trials or in development.

Other anti-HIV agents which may be used in coadministration with compounds according to the present disclosure include, for example, other NNRTI's (i.e., other than the NNRTI's according to the present disclosure) may be selected from the group consisting of nevirapine (BI-R6-587), delavirdine (U-90152S/T), efavirenz (DMP-266), UC-781 (N-[4-chloro-3-(3-methyl-2-butenyloxy)phenyl]-2methyl3-furancarbothiamide), etravirine (TMC125), Trovirdine (Ly300046.HCl), MKC-442 (emivirine, coactinon), HI-236, HI-240, HI-280, HI-281, rilpivirine (TMC-278), MSC-127, HBY 097, DMP266, Baicalin (TIN-151) ADAM-II (Methyl 3′,3′-dichloro-4′,4″-dimethoxy-5′,5″-bis(methoxycarbonyl)-6,6-diphenylhexenoate), Methyl 3-Bromo-5-(1-5-bromo-4-methoxy-3-(methoxycarbonyl)phenyl)hept-1-enyl)-2-methoxybenzoate (Alkenyldiarylmethane analog, Adam analog), (5-chloro-3-(phenylsulflnyl)-2′-indolecarboxamide), AAP-BHAP (U-104489 or PNU-104489), Capravirine (AG-1549, S-1153), atevirdine (U-87201E), aurin tricarboxylic acid (SD-095345), 1-[(6-cyano-2-indolyl)carbonyl]-4-[3-(isopropylamino)-2-pyridinyl]piperazine, 1-[5-[[N-(methyl)methylsulfonylamino]-2-indolylcarbonyl-4-[3-(isopropylamino)-2-pyridinyl]piperazine, 1-[3-(Ethylamino)-2-[pyridinyl]-4-[(5-hydroxy-2-indolyl)carbonyl]piperazine, 1-[(6-Formyl-2-indolyl)carbonyl]-4-[3-(isopropylamino)-2-pyridinyl]piperazine, 1-[[5-(Methylsulfonyloxy)-2-indoyly)carbonyl]-4-[3-(isopropylamino)-2-pyridinyl]piperazine, U88204E, Bis(2-nitrophenyl)sulfone (NSC 633001), Calanolide A (NSC675451), Calanolide B, 6-Benzyl-5-methyl-2-(cyclohexyloxy)pyrimidin-4-one (DABO-546), DPC 961, E-EBU, E-EBU-dm, E-EPSeU, E-EPU, Foscarnet (Foscavir), HEPT (l-[(2-Hydroxyethoxy)methyl]-6-(phenylthio)thymine), HEPT-M (1-[(2-Hydroxyethoxy)methyl]-6-(3-methylphenyl)thio)thymine), HEPT-S (1-[(2-Hydroxyethoxy)methyl]-6-(phenylthio)-2-thiothymine), Inophyllum P, L-737,126, Michellamine A (NSC650898), Michellamine B (NSC649324), Michellamine F, 6-(3,5-Dimethylbenzyl)-1-[(2-hydroxyethoxy)methyl]-5-isopropyluracil, 6-(3,5-Dimethylbenzyl)-1-(ethyoxymethyl)-5-isopropyluracil, NPPS, E-BPTU (NSC 648400), Oltipraz (4-Methyl-5-(pyrazinyl)-3H-1,2-dithiole-3-thione), N-{2-(2-Chloro-6-fluorophenethyl]-N′-(2-thiazolyl)thiourea (PETT Cl, F derivative), N-{2-(2,6-Difluorophenethyl]-N′-[2-(5-bromopyridyl)]thiourea {PETT derivative), N-{2-(2,6-Difluorophenethyl]-N′-[2-(5-methylpyridyl)]thiourea {PETT Pyridyl derivative), N-[2-(3-Fluorofuranyl)ethyl]-N′-[2-(5-chloropyridyl)]thiourea, N-[2-(2-Fluoro-6-ethoxyphenethyl)]—N′-[2-(5-bromopyridyl)]thiourea, N-(2-Phenethyl)-N′-(2-thiazolyl)thiourea (LY-73497), L-697,639, L-697,593, L-697,661, 3-[2-(4,7-Difluorobenzoxazol-2-yl)ethyl}-5-ethyl-6-methyl(pypridin-2(1H)-thione (2-Pyridinone Derivative), 3-[[(2-Methoxy-5,6-dimethyl-3-pyridyl)methyl]amine]-5-ethyl-6-methyl(pypridin-2(1H)-thione, R82150, R82913, R87232, R88703, R89439 (Loviride), R90385, S-2720, Suramin Sodium, TBZ (Thiazolobenzimidazole, NSC 625487), Thiazoloisoindol-5-one, (+)(R)-9b-(3,5-Dimethylphenyl-2,3-dihydrothiazolo[2,3-a]isoindol-5(9bH)-one, Tivirapine (R86183), UC-38 and UC-84, among others.

The term “pharmaceutically acceptable salt” is used throughout the specification to describe, where applicable, a salt form of one or more of the compounds described herein which are presented to increase the solubility of the compound in the gastric juices of the patient's gastrointestinal tract in order to promote dissolution and the bioavailability of the compounds. Pharmaceutically acceptable salts include those derived from pharmaceutically acceptable inorganic or organic bases and acids, where applicable. Suitable salts include those derived from alkali metals such as potassium and sodium, alkaline earth metals such as calcium, magnesium and ammonium salts, among numerous other acids and bases well known in the pharmaceutical art. Sodium and potassium salts are particularly preferred as neutralization salts of the phosphates according to the present disclosure.

The term “pharmaceutically acceptable derivative” is used throughout the specification to describe any pharmaceutically acceptable prodrug form (such as an ester, amide other prodrug group), which, upon administration to a patient, provides directly or indirectly the present compound or an active metabolite of the present compound.

### General Synthetic Approach

The synthetic realization and optimization of the bifunctional molecules as described herein may be approached in a step-wise or modular fashion. For example, identification of compounds that bind to the target molecules can involve high or medium throughput screening campaigns if no suitable ligands are immediately available. It is not unusual for initial ligands to require iterative design and optimization cycles to improve suboptimal aspects as identified by data from suitable in vitro and pharmacological and/or ADMET assays. Part of the optimization/SAR campaign would be to probe positions of the ligand that are tolerant of substitution and that might be suitable places on which to attach the linker chemistry previously referred to herein. Where crystallographic or NMR structural data are available, these can be used to focus such a synthetic effort.

In a very analogous way one can identify and optimize ligands for an E3 Ligase, i.e. ULMs/ILMs/VLMs/CLMs/ILMs.

With PTMs and ULMs (e.g. ILMs, VLMs, CLMs, and/or ILMs) in hand, one skilled in the art can use known synthetic methods for their combination with or without a linker moiety. Linker moieties can be synthesized with a range of compositions, lengths and flexibility and functionalized such that the PTM and ULM groups can be attached sequentially to distal ends of the linker. Thus a library of bifunctional molecules can be realized and profiled in in vitro and in vivo pharmacological and ADMET/PK studies. As with the PTM and ULM groups, the final bifunctional molecules can be subject to iterative design and optimization cycles in order to identify molecules with desirable properties.

In some instances, protecting group strategies and/or functional group interconversions (FGIs) may be required to facilitate the preparation of the desired materials. Such chemical processes are well known to the synthetic organic chemist and many of these may be found in texts such as “Greene's Protective Groups in Organic Synthesis” Peter G. M. Wuts and Theodora W. Greene (Wiley), and “Organic Synthesis: The Disconnection Approach” Stuart Warren and Paul Wyatt (Wiley).

### Protein Level Control

This description also provides methods for the control of protein levels with a cell. This is based on the use of compounds as described herein, which are known to interact with a specific target protein such that degradation of a target protein in vivo will result in the control of the amount of protein in a biological system, preferably to a particular therapeutic benefit.

The following examples are used to assist in describing the present disclosure, but should not be seen as limiting the present disclosure in any way.

### Specific Embodiments of the Present Disclosure

The present disclosure encompasses the following specific embodiments. These following embodiments may include all of the features recited in a proceeding embodiment, as specified. Where applicable, the following embodiments may also include the features recited in any proceeding embodiment inclusively or in the alternative (e.g., an eighth embodiment may include the features recited in a first embodiment, as recited, and/or the features of any of the second through seventh embodiments).

In certain embodiments, the description provides the following exemplary bifunctional molecules molecules that target Fak (compounds 100-109 of Table 1), including salts, prodrugs, polymorphs, analogs, derivatives, and deuterated forms thereof.

An aspect of the present disclosure provides a bifunctional compound having the chemical structure:

ULM-L-PTM,

or a pharmaceutically acceptable salt, enantiomer, stereoisomer, solvate, polymorph or prodrug thereof,

wherein:
the ULM is a small molecule E3 ubiquitin ligase binding moiety that binds an E3 ubiquitin ligase;
the PTM is a small molecule comprising a focal adhesion kinase protein targeting moiety; and
the L is a bond or a chemical linking moiety connecting the ULM and the PTM.

In any aspect or embodiment described herein, the E3 ubiquitin ligase binding moiety that targets an E3 ubiquitin ligase selected from the group consisting of Von Hippel-Lindau (VLM), cereblon (CLM), mouse double-minute homolog2 (MLM), and IAP (ILM).

In any aspect or embodiment described herein, the PTM is represented by:

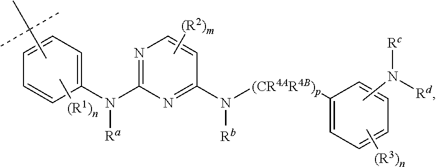

wherein:
each of R¹, R², R³, R^{4A}, R^{4B} is independently hydrogen, halogen, C₁-C₈ alkyl, C₁-C₈ haloalkyl, C₂-C₈ alkenyl, C₂-C₈ alkynyl, C₁-C₈ alkloxy, C₃-C₈ cycloalkyl, heterocycloalkyl, aryl, heteroaryl, primary amino, secondary amino, amido, carboxyl, acetyl or cyano;
each of R^{a}, R^{b}, R^{c}, and R^{d} is independently hydrogen, C₁-C₈ alkyl, C₁-C₈ haloalkyl, C₂-C₈ alkenyl, C₂-C₈ alkynyl, C₃-C₈ cycloalkyl, heterocycloalkyl, aryl, heteroaryl, or SO2-R^{e};
each R^{e} is independently C₁-C₄ alkyl;
each n is independently an integer from 0 to 4;
m is an integer from 0 to 2;
p is an integer from 1 to 8; and
the

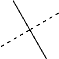

indicates the site of attachment of at least one of a linker, ULM, ULM′, CLM, CLM′, VLM, VLM′, ILM, ILM′, MLM, MLM′, or a combination thereof.

In any aspect or embodiment described herein, the PTM is represented by:

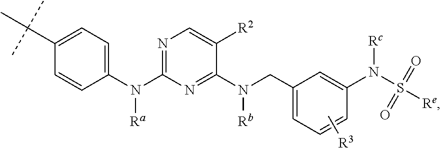

wherein:
R² is hydrogen, halogen, C₁-C₄ alkyl, or C₁-C₄ haloalkyl;
R³ is hydrogen, halogen, C₁-C₄ alkyl, C₁-C₄ alkoxy, or —CN;
each of R^{a}, R^{b}, and R^{c} are independently, hydrogen or C₁-C₄ alkyl;
R^{e} is C₁—O alkyl; and
the

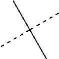

indicates the site of attachment of at least one of a linker, ULM, ULM′, CLM, CLM′, VLM, VLM′, ILM, ILM′, MLM, MLM′, or a combination thereof.

In any aspect or embodiment described herein, the PTM is represented by chemical structure:

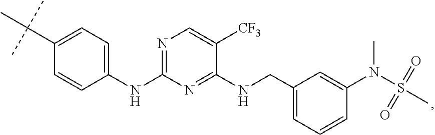

wherein the

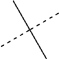

indicates the site of attachment of at least one of a linker, ULM, ULM′, CLM, CLM′, VLM, VLM′, ILM, ILM′, MLM, MLM′, or a combination thereof.

In any aspect or embodiment described herein, the ULM is a Von Hippel-Lindau (VHL) ligase-binding moiety (VLM) with a chemical structure represented by:

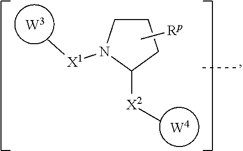

wherein:
X¹, X² are each independently selected from the group of a bond, O, NR^{Y3}, CR^{Y3}R^{Y4}, C═O, C═S, SO, and SO₂;
R^{Y3}, R^{Y4} are each independently selected from the group of H, linear or branched C₁₋₆ alkyl, optionally substituted by 1 or more halo, optionally substituted C₁₋₆ alkoxyl (e.g., optionally substituted by 0-3 R^{p} groups);
R^{p} is 0, 1, 2, or 3 groups, each independently selected from the group H, halo, —OH, C₁₋₃ alkyl, C═O;
W³ is selected from the group of an optionally substituted T, an optionally substituted -T-N(R^{1a}R^{1b})X³, an optionally substituted -T-N(R^{1a}R^{1b}), an optionally substituted -T-Aryl, an optionally substituted -T-Heteroaryl, an optionally substituted T-biheteroaryl, an optionally substituted -T-Heterocycle, an optionally substituted -T-biheterocycle, an optionally substituted —NR¹-T-Aryl, an optionally substituted —NR¹-T-Heteroaryl or an optionally substituted —NR¹-T-Heterocycle;
X³ is C═O, R¹, R^{1a}, R^{1b};
each of R¹, R^{1a}, R^{1b} is independently selected from the group consisting of H, linear or branched C₁-C₆ alkyl group optionally substituted by 1 or more halo or —OH groups, R^{Y3}C═O, R^{Y3}C═S, R^{Y3}SO, R^{Y3}SO₂, N(R^{Y3}R^{Y4})C═O, N(R^{Y3}R^{Y4})C═S, N(R^{Y3}R^{Y4})SO, and N(R^{Y3}R^{Y4})SO₂;
T is selected from the group of an optionally substituted alkyl, —(CH₂)ₙ— group, wherein each one of the methylene groups is optionally substituted with one or two substituents selected from the group of halogen, methyl, optionally substituted alkoxy, a linear or branched C₁-C₆ alkyl group optionally substituted by 1 or more halogen, C(O) NR¹R^{1a}, or NR¹R^{1a} or R¹ and R^{1a} are joined to form an optionally substituted heterocycle, or —OH groups or an amino acid side chain optionally substituted; and
n is 0 to 6,
W⁴ is

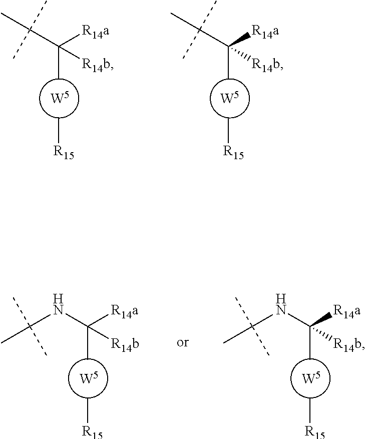

R₁₄ₐ, R_{14b}, are each independently selected from the group of H, haloalkyl, or optionally substituted alkyl;
W⁵ is selected from the group of an optionally substituted phenyl or an optionally substituted 5-10 membered heteroaryl,
R₁₅ is selected from the group of H, halogen, CN, OH, NO₂, NR₁₄ₐR_{14b}, OR₁₄ₐ, CONR₁₄ₐR_{14b}, NR₁₄ₐCOR_{14b}, SO₂NR₁₄ₐR_{14b}, NR₁₄ₐSO₂R_{14b}, optionally substituted alkyl, optionally substituted haloalkyl, optionally substituted haloalkoxy; aryl, heteroaryl, cycloalkyl, or cycloheteroalkyl (each optionally substituted);
and wherein the dashed line indicates the site of attachment of at least one PTM, another ULM (ULM′) or a chemical linker moiety coupling at least one PTM or a ULM′ or both to ULM.

In any aspect or embodiment described herein, the ULM is a Von Hippel-Lindau (VHL) ligase-binding moiety (VLM) with a chemical structure represented by:

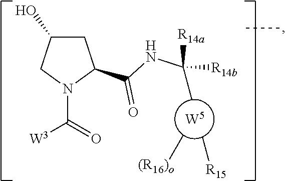

wherein:
W³ is selected from the group of an optionally substituted aryl, optionally substituted heteroaryl, or

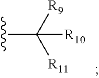

R₉ and R₁₀ are independently hydrogen, optionally substituted alkyl, optionally substituted cycloalkyl, optionally substituted hydroxyalkyl, optionally substituted heteroaryl, or haloalkyl, or R₉, R₁₀, and the carbon atom to which they are attached form an optionally substituted cycloalkyl;
R₁₁ is selected from the group of an optionally substituted heterocyclic, optionally substituted alkoxy, optionally substituted heteroaryl, optionally substituted aryl,

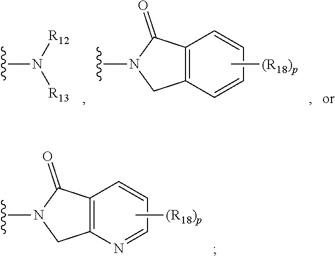

R₁₂ is selected from the group of H or optionally substituted alkyl;
R₁₃ is selected from the group of H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl;
R₁₄ₐ, R_{14b}, are each independently selected from the group of H, haloalkyl, or optionally substituted alkyl;
W⁵ is selected from the group of an optionally substituted phenyl or an optionally substituted 5-10 membered heteroaryl,
R₁₅ is selected from the group of H, halogen, CN, OH, NO₂, NR₁₄ₐR_{14b}, OR₁₄ₐ, CONR₁₄ₐR_{14b}, NR₁₄ₐCOR_{14b}, SO₂NR₁₄ₐR_{14b}, NR₁₄ₐSO₂R_{14b}, optionally substituted alkyl, optionally substituted haloalkyl, optionally substituted haloalkoxy, optionally substituted aryl, optionally substituted heteroaryl, optionally substituted cycloalkyl, or optionally substituted cycloheteroalkyl;
each R₁₆ is independently selected from the group of H, CN, halo, optionally substituted alkyl, optionally substituted haloalkyl, hydroxy, or optionally substituted haloalkoxy;
o is 0, 1, 2, 3, or 4;
R₁₈ is independently selected from the group of H, halo, optionally substituted alkoxy, cyano, optionally substituted alkyl, haloalkyl, haloalkoxy or a linker; and
p is 0, 1, 2, 3, or 4, and wherein the dashed line indicates the site of attachment of at least one PTM, another ULM (ULM′) or a chemical linker moiety coupling at least one PTM or a ULM′ or both to ULM.

In any aspect or embodiment described herein, the ULM has a chemical structure selected from the group of:

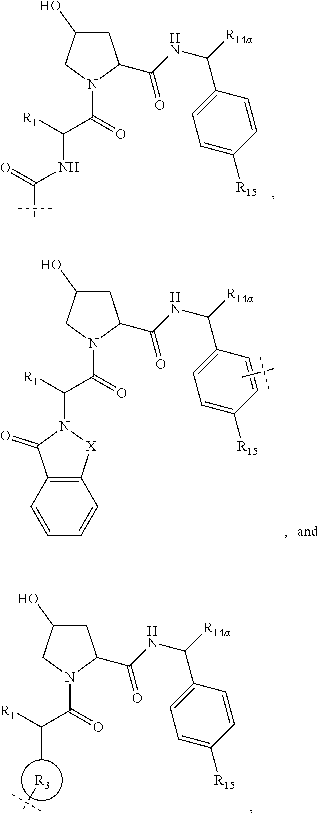

wherein:
R₁ is H, ethyl, isopropyl, tert-butyl, sec-butyl, cyclopropyl, cyclobutyl, cyclopentyl, or cyclohexyl; optionally substituted alkyl, optionally substituted hydroxyalkyl, optionally substituted heteroaryl, or haloalkyl;
R₁₄ₐ is H, haloalkyl, optionally substituted alkyl, methyl, fluoromethyl, hydroxymethyl, ethyl, isopropyl, or cyclopropyl;
R₁₅ is selected from the group consisting of H, halogen, CN, OH, NO₂, optionally substituted heteroaryl, optionally substituted aryl; optionally substituted alkyl, optionally substituted haloalkyl, optionally substituted haloalkoxy, optionally substituted cycloalkyl, or optionally substituted cycloheteroalkyl;
X is C, CH₂, or C═O
R₃ is absent or an optionally substituted 5 or 6 membered heteroaryl; and
wherein the dashed line indicates the site of attachment of at least one PTM, another ULM (ULM′) or a chemical linker moiety coupling at least one PTM or a ULM′ or both to the ULM.

In any aspect or embodiment described herein, the ULM comprises a group according to the chemical structure:

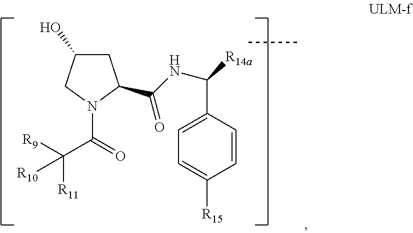

wherein:
R₁₄ₐ of Formula ULM-f is H, haloalkyl, optionally substituted alkyl, methyl, fluoromethyl, hydroxymethyl, ethyl, isopropyl, or cyclopropyl;
R₉ of Formula ULM-f is H;
R₁₀ of Formula ULM-f is H, ethyl, isopropyl, tert-butyl, sec-butyl, cyclopropyl, cyclobutyl, cyclopentyl, or cyclohexyl;
R₁₁ of Formula ULM-f is

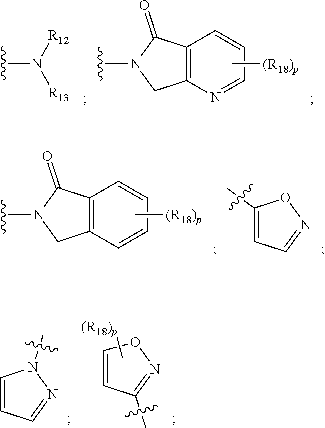

or optionally substituted heteroaryl;
p of Formula ULM-f is 0, 1, 2, 3, or 4;
each Rig of Formula ULM-f is independently halo, optionally substituted alkoxy, cyano, optionally substituted alkyl, haloalkyl, haloalkoxy or a linker;
R₁₂ of Formula ULM-f is H, C═O;
R₁₃ of Formula ULM-f is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl,
R₁₅ of Formula ULM-f is selected from the group consisting of H, halogen, Cl, CN, OH, NO₂, optionally substituted heteroaryl, optionally substituted aryl;

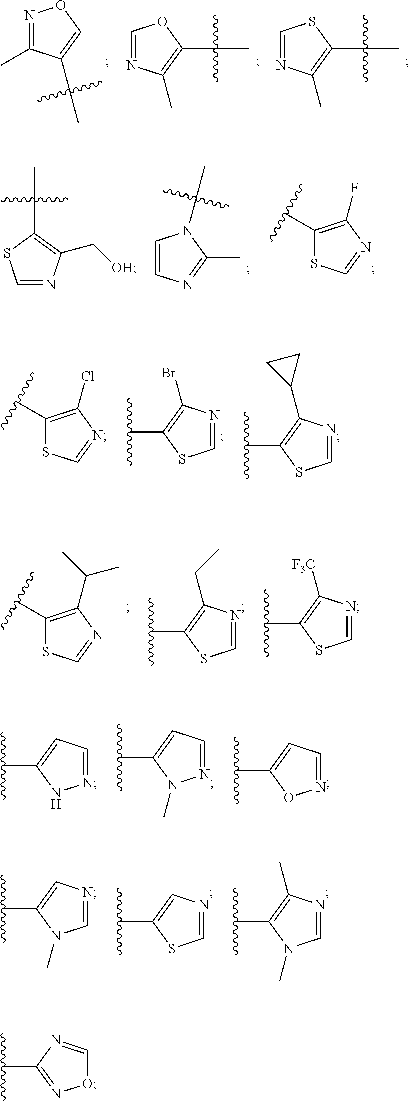

and
the dashed line of Formula ULM-f indicates the site of attachment of at least one PTM, another ULM (ULM′) or a chemical linker moiety coupling at least one PTM or a ULM′ or both to ULM.

In any aspect or embodiment described herein, the ULM comprises a group according to the chemical structure:

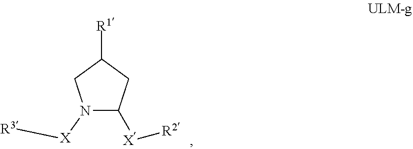

wherein:
R¹ of ULM-g is an optionally substituted C₁-C₆ alkyl group, an optionally substituted —(CH₂)ₙOH, an optionally substituted —(CH₂)ₙSH, an optionally substituted (CH₂)ₙ—O—(C₁-C₆)alkyl group, an optionally substituted (CH₂)ₙ—WCOCW—(C₀-C₆)alkyl group containing an epoxide moiety WCOCW where each W is independently H or a C₁-C₃ alkyl group, an optionally substituted —(CH₂)ₙCOOH, an optionally substituted —(CH₂)ₙC(O)—(C₁-C₆ alkyl), an optionally substituted —(CH₂)ₙNHC(O)—R₁, an optionally substituted —(CH₂)ₙC(O)—NR₁R₂, an optionally substituted —(CH₂)ₙOC(O)—NR₁R₂, —(CH₂O)ₙH, an optionally substituted —(CH₂)ₙOC(O)—(C₁-C₆ alkyl), an optionally substituted —(CH₂)ₙC(O)—O—(C₁-C₆ alkyl), an optionally substituted —(CH₂O)ₙCOOH, an optionally substituted —(OCH₂)ₙO—(C₁-C₆ alkyl), an optionally substituted —(CH₂O)ₙC(O)—(C₁-C₆ alkyl), an optionally substituted —(OCH₂)ₙNHC(O)—R₁, an optionally substituted —(CH₂O)ₙC(O)—NR₁R₂, —(CH₂CH₂O)ₙH, an optionally substituted —(CH₂CH₂O)ₙCOOH, an optionally substituted —(OCH₂CH₂)ₙO—(C₁-C₆ alkyl), an optionally substituted —(CH₂CH₂O)ₙC(O)—(C₁-C₆ alkyl), an optionally substituted —(OCH₂CH₂)ₙNHC(O)—R₁, an optionally substituted —(CH₂CH₂O)ₙC(O)—NR₁R₂, an optionally substituted —SO₂R₅, an optionally substituted S(O)R₅, NO₂, CN or halogen (F, Cl, Br, I, preferably F or Cl);
R₁ and R₂ of ULM-g are each independently H or a C₁-C₆ alkyl group which may be optionally substituted with one or two hydroxyl groups or up to three halogen groups (preferably fluorine);
R_{S} of ULM-g is a C₁-C₆ alkyl group, an optionally substituted aryl, heteroaryl or heterocycle group or a —(CH₂)ₘNR₁R₂ group;
X and X′ of ULM-g are each independently C═O, C═S, —S(O), S(O)₂, (preferably X and X′ are both C═O);
R² of ULM-g is an optionally substituted —(CH₂)ₙ—(C═O)ᵤ(NR₁)ᵥ(SO₂)_{w}alkyl group, an optionally substituted —(CH₂)_{D}—(C═O)ᵤ(NR₁)ᵥ(SO₂)_{w}NR_{1N}R_{2N} group, an optionally substituted —(CH₂)ₙ—(C═O)ᵤ(NR₁)ᵥ(SO₂)_{w}-Aryl, an optionally substituted —(CH₂)ₙ—(C═O)ᵤ(NR₁)ᵥ(SO₂)_{w}-Heteroaryl, an optionally substituted —(CH₂)ₙ—(C═O)ᵥNR₁(SO₂)_{w}-Heterocycle, an optionally substituted —NR¹—(CH₂)ₙ—C(O)ᵤ(NR₁)ᵥ(SO₂)_{w}-alkyl, an optionally substituted —NR¹—(CH₂)ₙ—C(O)ᵤ(NR₁)ᵥ(SO₂)_{w}—NR_{1N}R_{2N}, an optionally substituted —NR¹—(CH₂)ₙ—C(O)ᵤ(NR₁)ᵥ(SO₂)_{w}—NR₁C(O)R_{1N}, an optionally substituted —NR¹—(CH₂)ₙ—(C═O)ᵤ(NR₁)ᵥ(SO₂)_{w}-Aryl, an optionally substituted —NR¹—(CH₂)ₙ—(C═O)ᵤ(NR₁)ᵥ(SO₂)_{w}-Heteroaryl or an optionally substituted —NR¹—(CH₂)ₙ—(C═O)ᵥNR₁(SO₂)_{w}—Heterocycle, an optionally substituted —X^{R2}-alkyl group; an optionally substituted —X^{R2′}-Aryl group; an optionally substituted —X^{R2′}-Heteroaryl group; an optionally substituted —X^{R2′}-Heterocycle group; an optionally substituted;
R^{3′} of ULM-g is an optionally substituted alkyl, an optionally substituted —(CH₂)ₙ—(O)ᵤ(NR₁)ᵥ(SO₂)_{w}-alkyl, an optionally substituted —(CH₂)ₙ—C(O)ᵤ(NR₁)ᵥ(SO₂)_{w}—NR_{1N}R_{2N}, an optionally substituted —(CH₂)ₙ—C(O)ᵤ(NR₁)ᵥ(SO₂)_{w}—NR₁C(O)R_{1N}, an optionally substituted —(CH₂)ₙ—C(O)ᵤ(NR₁)ᵥ(SO₂)_{w}—C(O)NR₁R₂, an optionally substituted —(CH₂)ₙ—C(O)ᵤ(NR₁)ᵥ(SO₂)_{w}-Aryl, an optionally substituted —(CH₂)ₙ—C(O)ᵤ(NR₁)ᵥ(SO₂)_{w}-Heteroaryl, an optionally substituted —(CH₂)ₙ—C(O)ᵤ(NR₁)ᵥ(SO₂)_{w}-Heterocycle, an optionally substituted —NR¹—(CH₂)ₙ—C(O)ᵤ(NR₁)ᵥ(SO₂)_{w}-alkyl, an optionally substituted —NR¹—(CH₂)ₙ—C(O)ᵤ(NR₁)ᵥ(SO₂)_{w}—NR_{1N}R_{2N}, an optionally substituted —NR¹—(CH₂)ₙ—C(O)ᵤ(NR₁)ᵥ(SO₂)_{w}—NR₁C(O)R_{1N}, an optionally substituted —NR¹—(CH₂)ₙ—C(O)ᵤ(NR₁)ᵥ(SO2)_{w}-Aryl, an optionally substituted —NR¹—(CH₂)ₙ—C(O)ᵤ(NR₁)ᵥ(SO₂)_{w}-Heteroaryl, an optionally substituted —NR¹—(CH₂)ₙ—C(O)ᵤ(NR₁)ᵥ(SO₂)_{w}-Heterocycle, an optionally substituted —O—(CH₂)ₙ—(C═O)ᵤ(NR₁)ᵥ(SO₂)_{w}-alkyl, an optionally substituted —O—(CH₂)ₙ—(C═O)ᵤ(NR₁)ᵥ(SO₂)_{w}—NR_{1N}R_{2N}, an optionally substituted —O—(CH₂)ₙ—(C═O)ᵤ(NR₁)ᵥ(SO₂)_{w}—NR₁C(O)R_{1N}, an optionally substituted —O—(CH₂)ₙ—(C═O)ᵤ(NR₁)ᵥ(SO₂)_{w}-Aryl, an optionally substituted —O—(CH₂)ₙ—(C═O)ᵤ(NR₁)ᵥ(SO₂)_{w}-Heteroaryl or an optionally substituted —O—(CH₂)ₙ—(C═O)ᵤ(NR₁)ᵥ(SO₂)_{w}-Heterocycle; —(CH₂)ₙ—(V)_{n′}—(CH₂)ₙ—(V)_{n′}-alkyl group, an optionally substituted —(CH₂)ₙ—(V)_{n′}—(CH₂)ₙ—(V)_{n′}-Aryl group, an optionally substituted —(CH₂)ₙ—(V)_{n′}—(CH₂)ₙ—(V)_{n′}-Heteroaryl group, an optionally substituted —(CH₂)ₙ—(V)_{n′}—(CH₂)ₙ—(V)_{n′}-Heterocycle group, an optionally substituted —(CH₂)ₙ—N(R_{1′})(C═O)_{m′}—(V)_{n′}-alkyl group, an optionally substituted —(CH₂)ₙ—N(R_{1′})(C═O)_{m′}—(V)_{n′}-Aryl group, an optionally substituted —(CH₂)ₙ—N(R_{1′})(C═O)_{m′}—(V)_{n′}-Heteroaryl group, an optionally substituted —(CH₂)ₙ—N(R_{1′})(C═O)_{m′}—(V)_{n′}-Heterocycle group, an optionally substituted —X^{R3′}-alkyl group; an optionally substituted —X^{R3′}-Aryl group; an optionally substituted —X^{R3′}-Heteroaryl group; an optionally substituted —X^{R3′}-Heterocycle group; an optionally substituted;
R_{1N} and R_{2N} of ULM-g are each independently H, C₁-C₆ alkyl which is optionally substituted with one or two hydroxyl groups and up to three halogen groups or an optionally substituted —(CH₂)ₙ-Aryl, —(CH₂)ₙ-Heteroaryl or —(CH₂)ₙ-Heterocycle group;
V of ULM-g is O, S or NR₁;
R₁ of ULM-g is the same as above;
R¹ and R_{1′} of ULM-g are each independently H or a C₁-C₃ alkyl group;
X^{R2′} and X^{R3′} of ULM-g are each independently an optionally substituted —CH₂)ₙ—, —CH₂)ₙ—CH(Xᵥ)═CH(Xᵥ)-(cis or trans), —CH₂)ₙ—CH≡CH—, —(CH₂CH₂O)ₙ— or a C₃-C₆ cycloalkyl group, where Xᵥ is H, a halo or a C₁-C₃ alkyl group which is optionally substituted;
each m of ULM-g is independently 0, 1, 2, 3, 4, 5, 6;
each m′ of ULM-g is independently 0 or 1;
each n of ULM-g is independently 0, 1, 2, 3, 4, 5, 6;
each n′ of ULM-g is independently 0 or 1;
each u of ULM-g is independently 0 or 1;
each v of ULM-g is independently 0 or 1;
each w of ULM-g is independently 0 or 1; and
any one or more of R^{1′}, R^{2′}, R^{3′}, X and X′ of ULM-g is optionally modified to be covalently bonded to the PTM group through a linker group when PTM is not ULM′, or when PTM is ULM′, any one or more of R^{1′}, R^{2′}, R^{3′}, X and X′ of each of ULM and ULM′ are optionally modified to be covalently bonded to each other directly or through a linker group, or a pharmaceutically acceptable salt, stereoisomer, solvate or polymorph thereof.

In any aspect or embodiment described herein, the ULM is a cereblon E3 ligase-binding moiety (CLM) selected from the group consisting of a thalidomide, lenalidomide, pomalidomide, analogs thereof, isosteres thereof, or derivatives thereof.

In any aspect or embodiment described herein, the CLM has a chemical structure represented by:

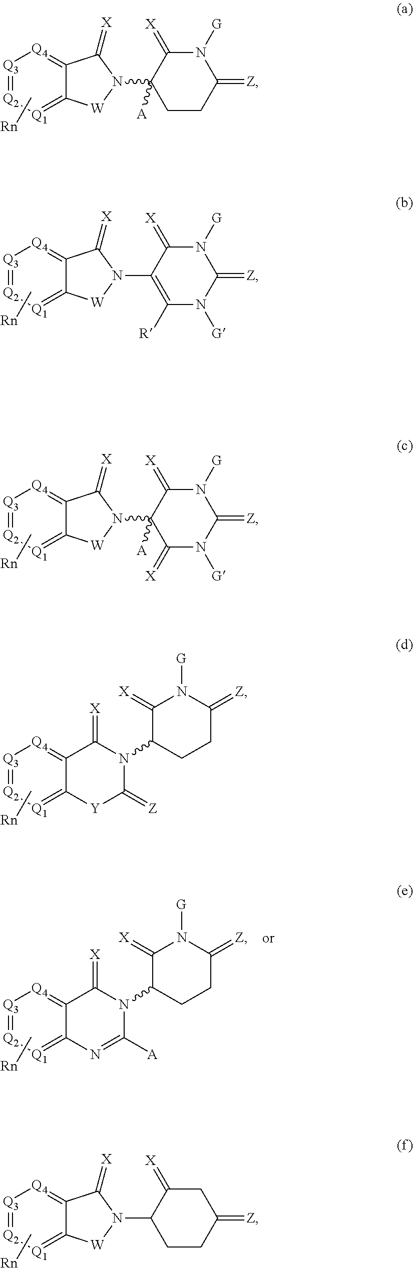

wherein:
W is selected from the group consisting of CH₂, CHR, C═O, SO₂, NH, and N-alkyl;
each X is independently selected from the group consisting of absent, O, S, and CH₂;
Y is selected from the group consisting of CH₂, —C═CR′, NH, N-alkyl, N-aryl, N-heteroaryl, N-cycloalkyl, N-heterocyclyl, O, and S;
Z is selected from the group consisting of absent, O, S, and CH₂;
G and G′ are independently selected from the group consisting of H, optionally substituted linear or branched alkyl, OH, R′OCOOR, R′OCONRR″, CH₂-heterocyclyl optionally substituted with R′, and benzyl optionally substituted with R′;
Q₁, Q₂, Q₃, and Q₄ represent a carbon C substituted with a group independently selected from H, R, N or N-oxide;
A is independently selected from the group H, alkyl, cycloalkyl, Cl and F;
R comprises —CONR′R″, —OR′, —NR′R″, —SR′, —SO₂R′, —SO₂NR′R″, —CR′R″—, —CR′NR′R″—, (—CR′O)_{n′}R″, optionally substituted heterocyclyl, optionally substituted-aryl, optionally substituted-hetaryl, unsubstituted or substituted linear or branched alkyl, optionally substituted-cycloalkyl, optionally substituted-heterocyclyl, —P(O)(OR′)R″, —P(O)R′R″, —OP(O)(OR′)R″, —OP(O)R′R″, —C₁, —F, —Br, —I, —CF₃, —CN, —NR′SO₂NR′R″, —NR′CONR′R″, —CONR′COR″, —NR′C(═N—CN)NR′R″, —C(═N—CN)NR′R″, —NR′C(═N—CN)R″, —NR′C(═C—NO₂)NR′R″, —SO₂NR′COR″, —NO₂, —CO₂R′—C(C═N—OR′)R″, —CR′═CR′R″, —CCR′—S(C═O)(C═N—R′)R″, —SF₅ and —OCF₃, wherein at least one R (e.g., at least one of O, OH, H, NH, NH₂, C₁-C₆ alkyl, C₁-C₆ alkoxy, -alkyl-aryl (e.g., an -alkyl-aryl comprising at least one of C₁-C₆ alkyl, C₄-C₇ aryl, or a combination thereof), aryl (e.g., C₅-C₇ aryl), amine, amide, or carboxy) is modified to be covalently joined to a PTM, a chemical linker group (L), a ULM, a CLM′ (e.g., CLM′ is an additional CLM that has the same or different structure as a first CLM), or a combination thereof;
R′ and R″ are independently selected from the group consisting of a H, optionally substituted linear or branched alkyl, optionally substituted cycloalkyl, optionally substituted aryl, optionally substituted heteroaryl, optionally substituted heterocyclyl;
n and n′ of Formulas (a) through (f) are independently an integer from 1-10 (e.g. 1-4, 1, 2, 3, 4, 5, 6, 7, 8, 9, or 10);
[symbol] represents a bond that may be stereospecific ((R) or (S)) or non-stereospecific.

In any aspect or embodiment described herein, the CLM has a chemical structure represented by:

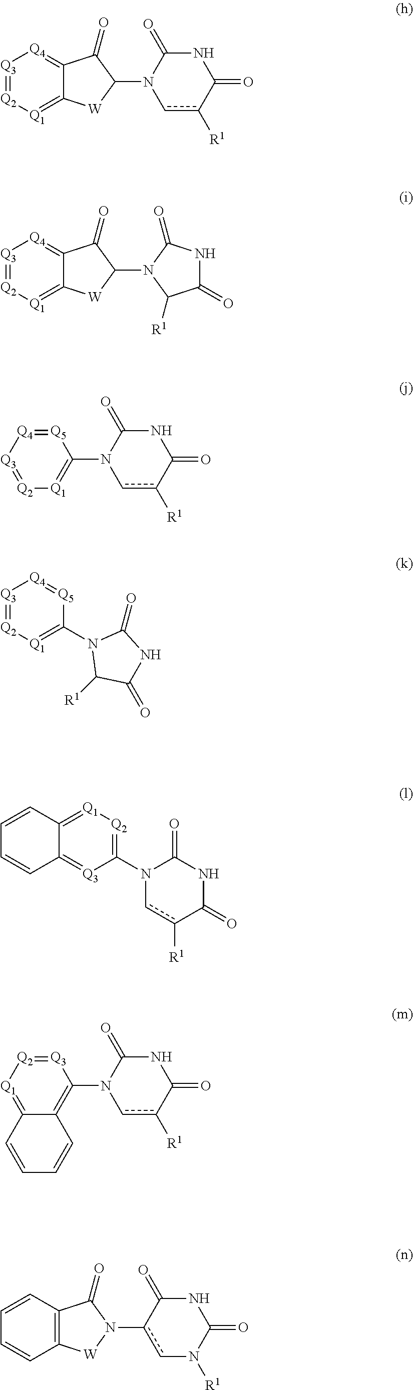

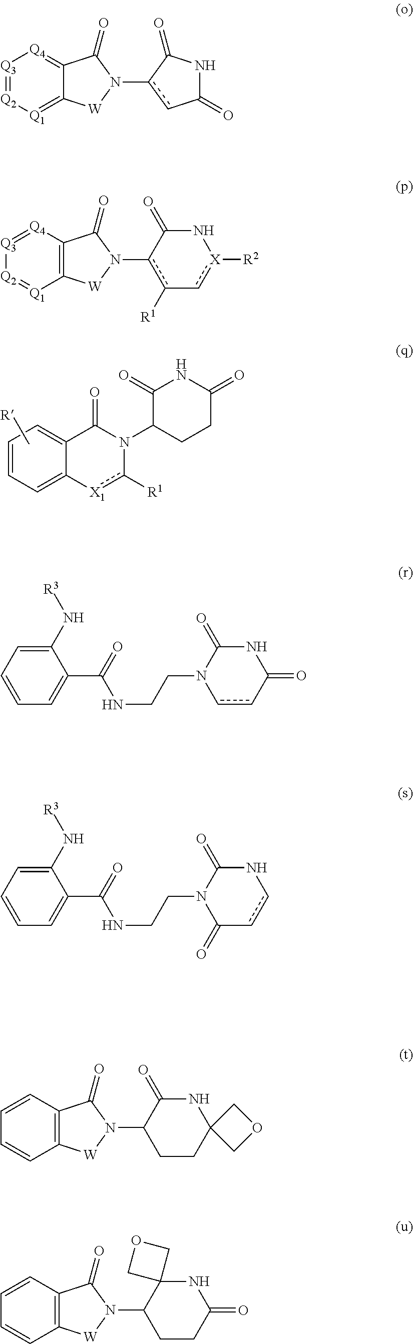

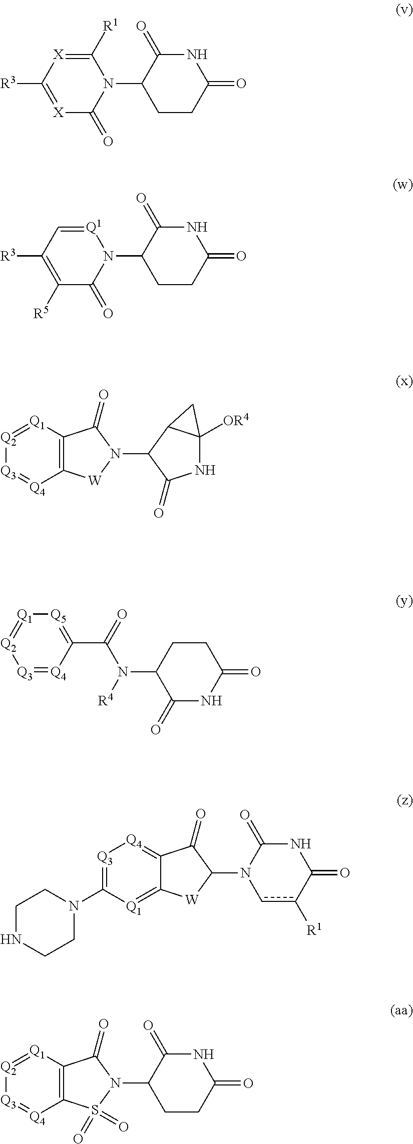

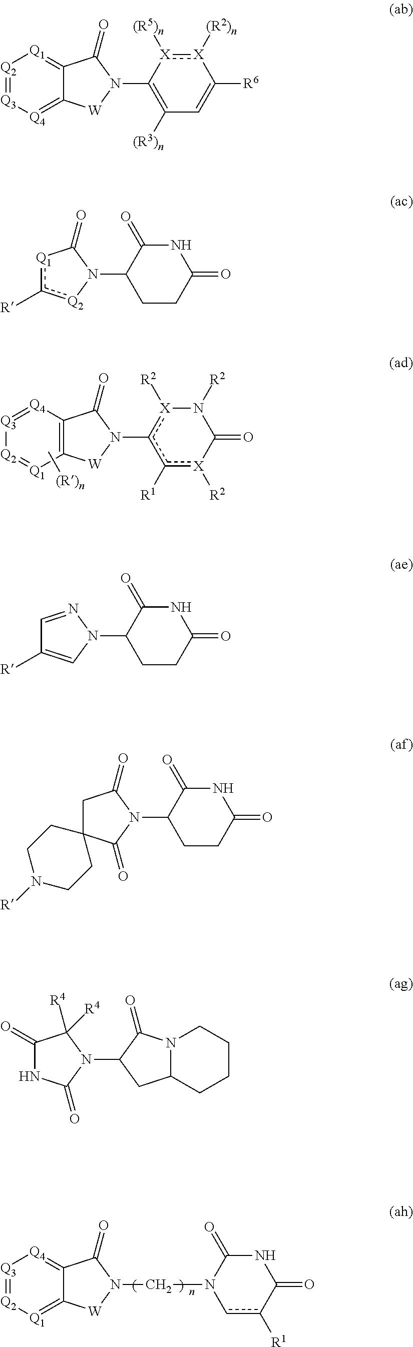

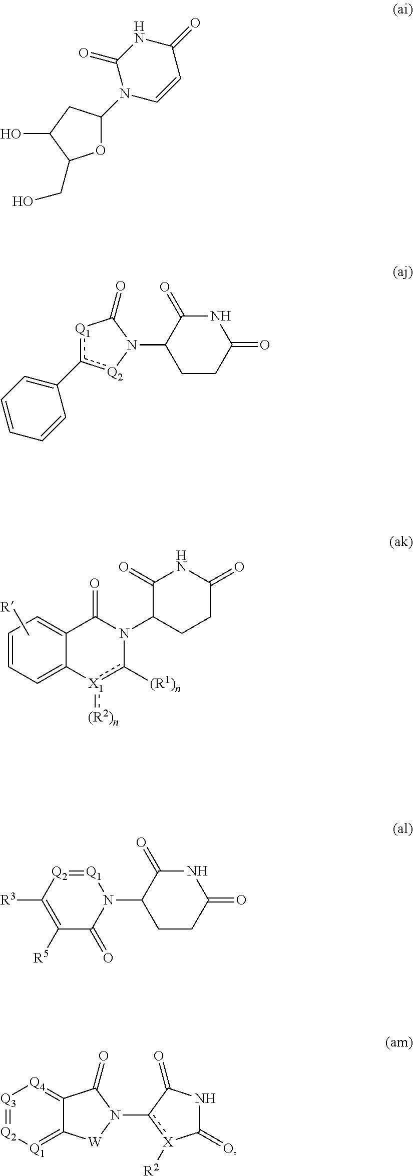

wherein:
W is independently selected from CH₂, CHR, C═O, SO₂, NH, and N-alkyl;
Q₁, Q₂, Q₃, Q₄, Q₅ are each independently represent a carbon C or N substituted with a group independently selected from R′, N or N-oxide;
R¹ is selected from absent, H, OH, CN, C₁-C₃ alkyl, C═O;
R² is selected from the group absent, H, OH, CN, C₁-C₃ alkyl, CHF₂, CF₃, CHO, C(═O)NH₂;
R³ is selected from H, alkyl (e.g., C₁-C₆ or C₁-C₃ alkyl), substituted alkyl (e.g., substituted C₁-C₆ or C₁-C₃ alkyl), alkoxy (e.g., C₁-C₆ or C₁-C₃ alkoxyl), substituted alkoxy (e.g., substituted C₁-C₆ or C₁-C₃ alkoxyl);
R⁴ is selected from H, alkyl, substituted alkyl;
R⁵ and R⁶ are each independently H, halogen, C(═O)R′, CN, OH, CF₃;
X is C, CH, C═O, or N;
X₁ is C═O, N, CH, or CH₂;
R′ is selected from H, halogen, amine, alkyl (e.g., C₁-C₃ alkyl), substituted alkyl (e.g., substituted C₁-C₃ alkyl), alkoxy (e.g., C₁-C₃ alkoxyl), substituted alkoxy (e.g., substituted C₁-C₃ alkoxyl), NR²R³, C(═O)OR², optionally substituted phenyl;
each n is independently an integer from 0-4;
[symbol] is a single or double bond; and
the CLM is covalently joined to a PTM, a chemical linker group (L), a ULM, CLM (or CLM′) or combination thereof.

In any aspect or embodiment described herein, the ULM is a (MDM2) binding moiety (MLM) with a chemical moiety selected from the group consisting of a substituted imidazolines, a substituted spiro-indolinones, a substituted pyrrolidines, a substituted piperidinones, a substituted morpholinones, a substituted pyrrolopyrimidines, a substituted imidazolopyridines, a substituted thiazoloimidazoline, a substituted pyrrolopyrrolidinones, and a substituted isoquinolinones.

In any aspect or embodiment described herein, the ULM is a IAP E3 ubiquitin ligase binding moiety (ILM) comprising the amino acids alanine (A), valine (V), proline (P), and isoleucine (I) or their unnatural mimetics.

In any aspect or embodiment described herein, the ULM is a IAP E3 ubiquitin ligase binding moiety (ILM) comprising a AVPI tetrapeptide fragment or derivative thereof.

In any aspect or embodiment described herein, the linker (L) comprises a chemical structural unit represented by the formula:

-(A^{L})_{q}-,

wherein:
(A^{L})_{q}- is a group which is connected to at least one of ULM, PTM, or both;
q is an integer greater than or equal to 1;
each A is independently selected from the group consisting of, a bond, CR^{L1}R^{L2}, O, S, SO, SO₂, NR^{L3}, SO₂NR^{L3}, SONR^{L3}, CONR^{L3}, NR^{L3}CONR^{L4}, NR^{L3}SO₂NR^{L4}, CO, CR^{L1}═CR^{L2}, C≡C, SiR^{L1}R^{L2}, P(O)R^{L1}, P(O)OR^{L1}, NR^{L3}C(═NCN)NR^{L4}, NR^{L3}C(═NCN), NR^{L3}C(═CNO₂)NR^{L4}, C₃₋₁₁cycloalkyl optionally substituted with 0-6 R^{L1} and/or R^{L2} groups, C₃₋₁₁heterocyclyl optionally substituted with 0-6 R^{L1} and/or R^{L2} groups, aryl optionally substituted with 0-6 R^{L1} and/or R^{L2} groups, heteroaryl optionally substituted with 0-6 R^{L1} and/or R^{L2} groups, where R^{L1} or R^{L2}, each independently are optionally linked to other groups to form cycloalkyl and/or heterocyclyl moiety, optionally substituted with 0-4 R^{L5} groups; and
R^{L1}, R^{L2}, R^{L3}, R^{L4} and R^{L5} are, each independently, H, halo, C₁₋₈alkyl, OC₁₋₈alkyl, SC₁₋₈alkyl, NHC₁₋₈alkyl, N(C₁₋₈alkyl)₂, C₃₋₁₁cycloalkyl, aryl, heteroaryl, C₃₋₁₁ heterocyclyl, OC₃₋₈cycloalkyl, SC₃₋₈cycloalkyl, NHC₃₋₈cycloalkyl, N(C₃₋₈cycloalkyl)₂, N(C₁₋₈cycloalkyl)(C₁₋₈alkyl), OH, NH₂, SH, SO₂C₁₋₈alkyl, P(O)(OC₁₋₈alkyl)(C₁₋₈alkyl), P(O)(OC₁₋₈alkyl)₂, CC—C₁₋₈alkyl, CCH, CH═CH(C₁₋₈alkyl), C(C₁₋₈alkyl)═CH(C₁₋₈alkyl), C(C₁₋₈alkyl)═C(C₁₋₈alkyl)₂, Si(OH)₃, Si(C₁₋₈alkyl)₃, Si(OH)(C₁₋₈alkyl)₂, COC₁₋₈alkyl, CO₂H, halogen, CN, CF₃, CHF₂, CH₂F, NO₂, SF₅, SO₂NHC₁₋₈alkyl, SO₂N(C₁₋₈alkyl)₂, SONHC₁₋₈alkyl, SON(C₁₋₈alkyl)₂, CONHC₁₋₈alkyl, CON(C₁₋₈alkyl)₂, N(C₁₋₈alkyl)CONH(C₁₋₈alkyl), N(C₁₋₈alkyl)CON(C₁₋₈alkyl)₂, NHCONH(C₁₋₈alkyl), NHCON(C₁₋₈alkyl)₂, NHCONH₂, N(C₁₋₈alkyl)SO₂NH(C₁₋₈alkyl), N(C₁₋₈alkyl) SO₂N(C₁₋₈alkyl)₂, NHSO₂NH(C₁₋₈alkyl), NHSO₂N(C₁₋₈alkyl)₂, NHSO₂NH₂.

In any aspect or embodiment described herein, the unit A^{L} of linker (L) comprises a group represented by a general structure selected from the group consisting of:
—N(R)—(CH₂)ₘ—O(CH₂)ₙ—O(CH₂)ₒ—O(CH₂)ₚ—O(CH₂)_{q}—O(CH₂)ᵣ—OCH₂—,
—O—(CH₂)ₘ—O(CH₂)ₙ—O(CH₂)ₒ—O(CH₂)ₚ—O(CH₂)_{q}—O(CH₂)ᵣ—OCH₂—,
—O—(CH₂)ₘ—O(CH₂)ₙ—O(CH₂)ₒ—O(CH₂)ₚ—O(CH₂)_{q}—O(CH₂)ᵣ—O—;
—N(R)—(CH₂)ₘ—O(CH₂)ₙ—O(CH₂)ₒ—O(CH₂)ₚ—O(CH₂)_{q}—O(CH₂)ᵣ—O—;
—(CH₂)ₘ—O(CH₂)ₙ—O(CH₂)ₒ—O(CH₂)ₚ—O(CH₂)_{q}—O(CH₂)ᵣ—O—;
—(CH₂)ₘ—O(CH₂)ₙ—O(CH₂)ₒ—O(CH₂)ₚ—O(CH₂)_{q}—O(CH₂)ᵣ—OCH₂—;

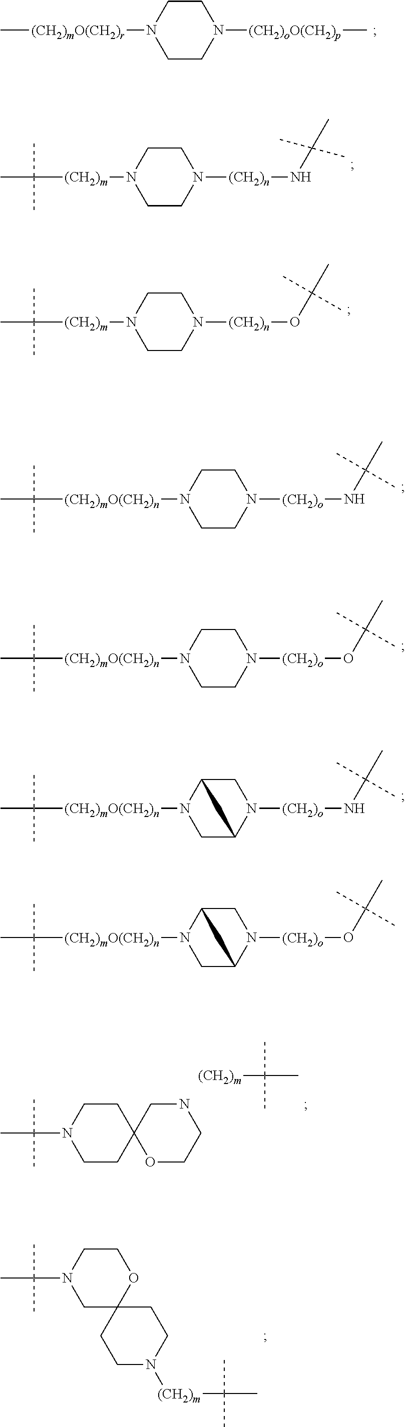

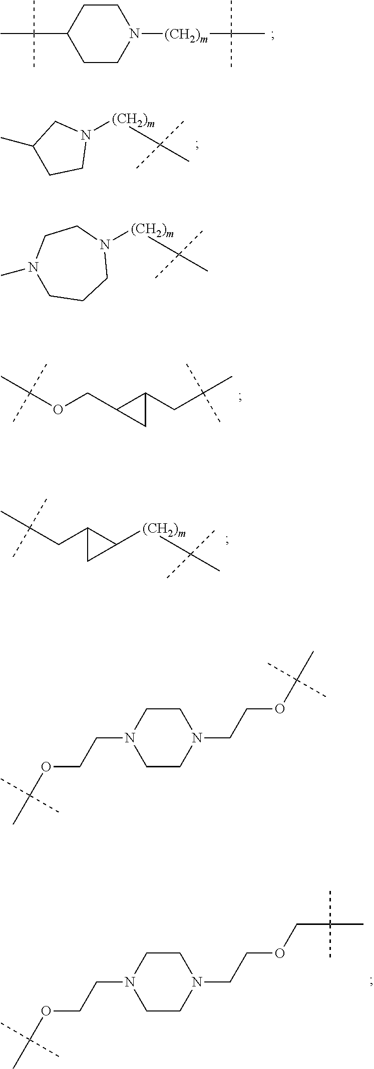

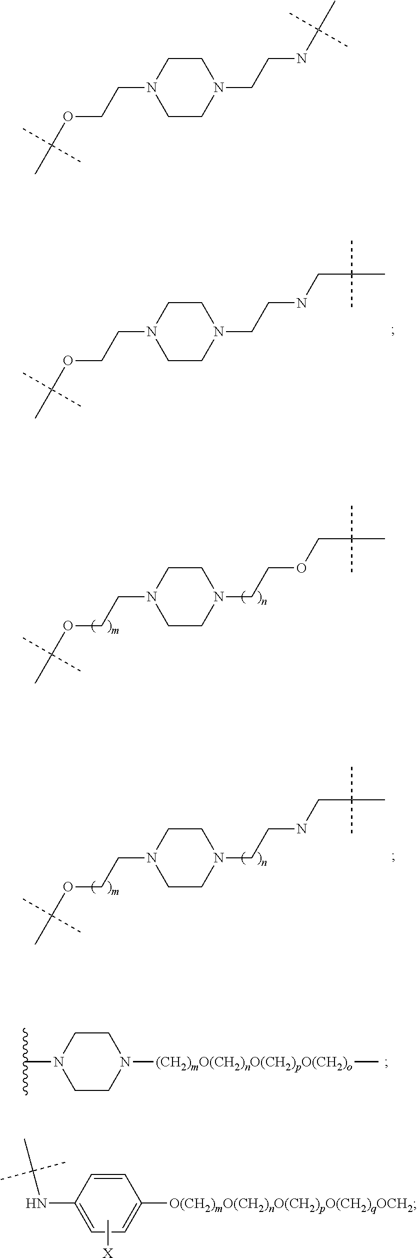

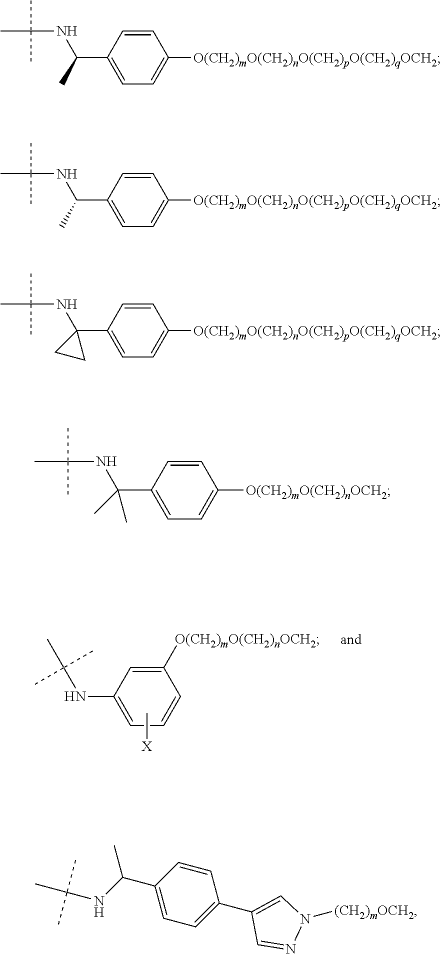

wherein each m, n, o, p, q, and r, is independently 0, 1, 2, 3, 4, 5, 6, 7, 8, 9, 10, 11, 12, 13, 14, 15, 16, 17, 18, 19, or 20 with the proviso that when the number is zero, there is no N—O or O—O bond, R is selected from the group H, methyl and ethyl, and X is selected from the group H and F;

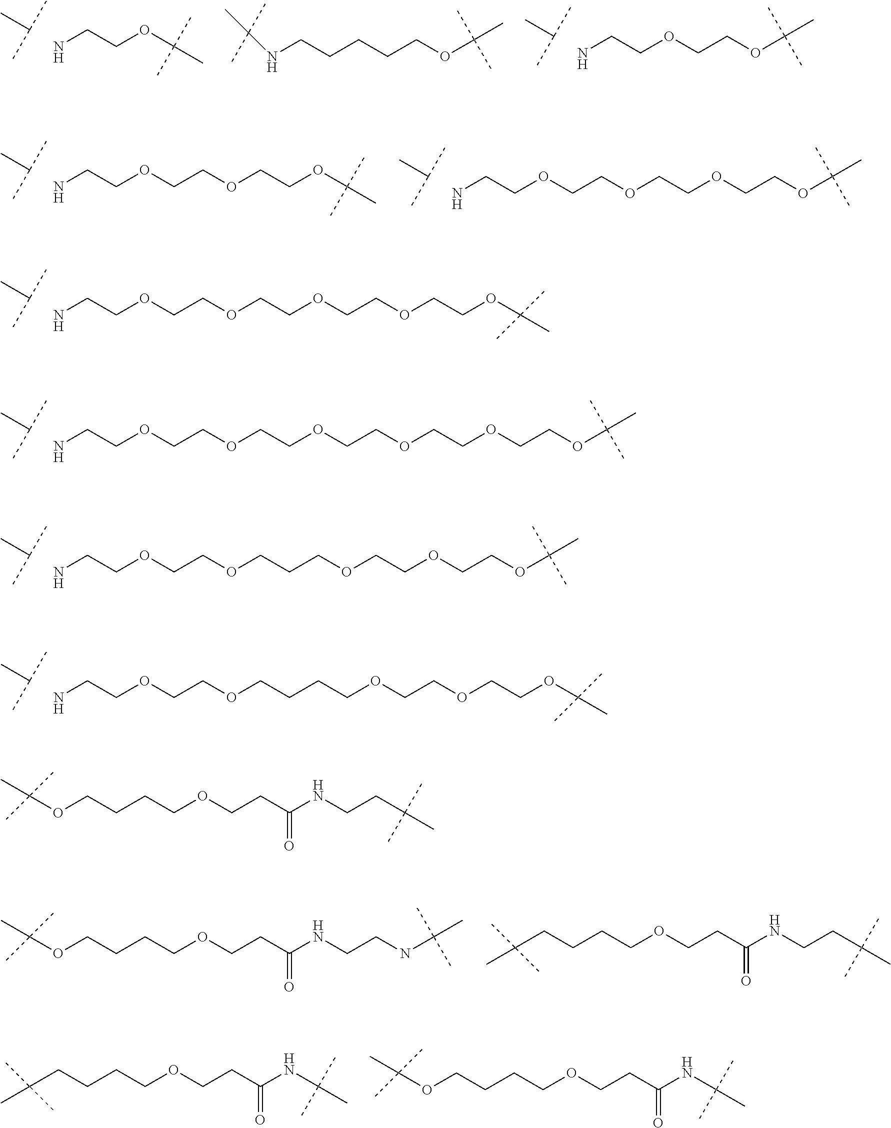

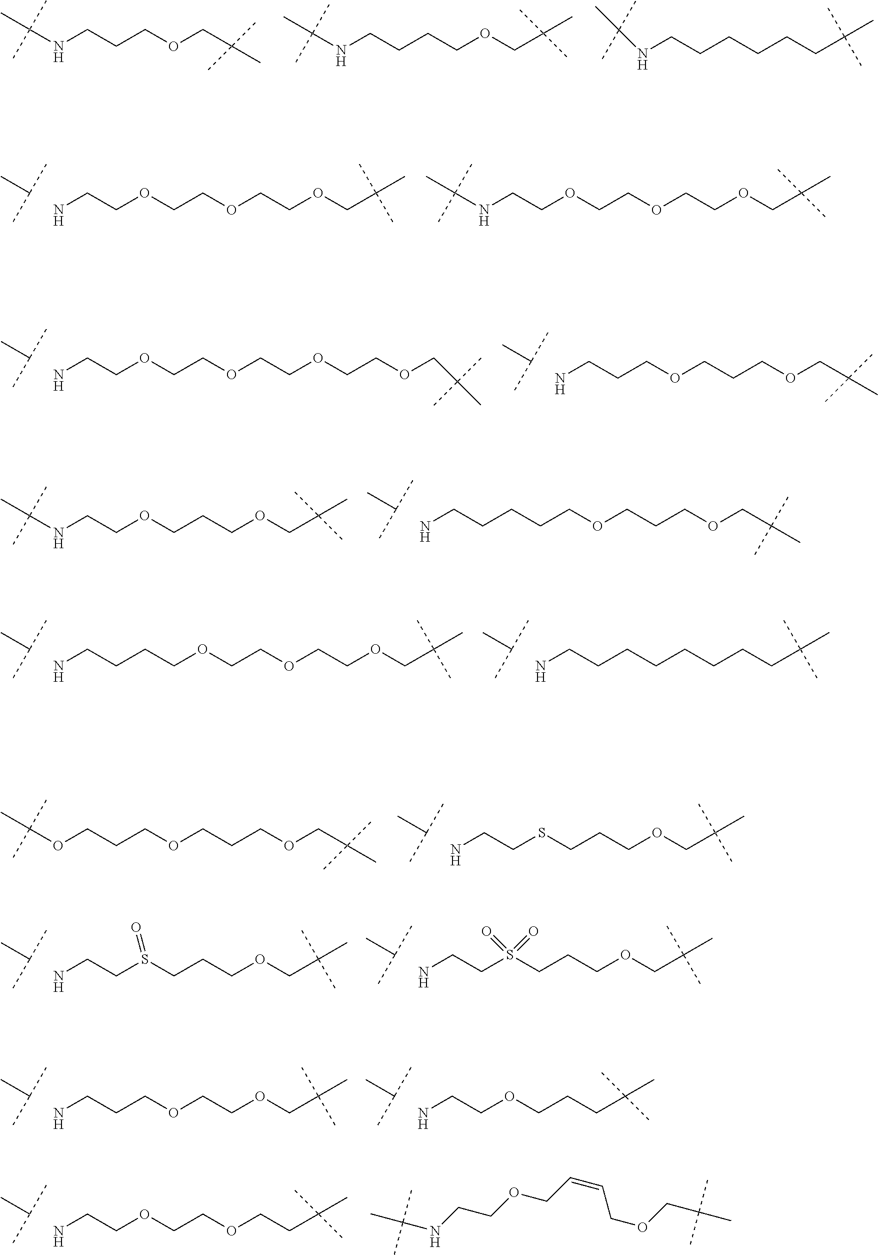

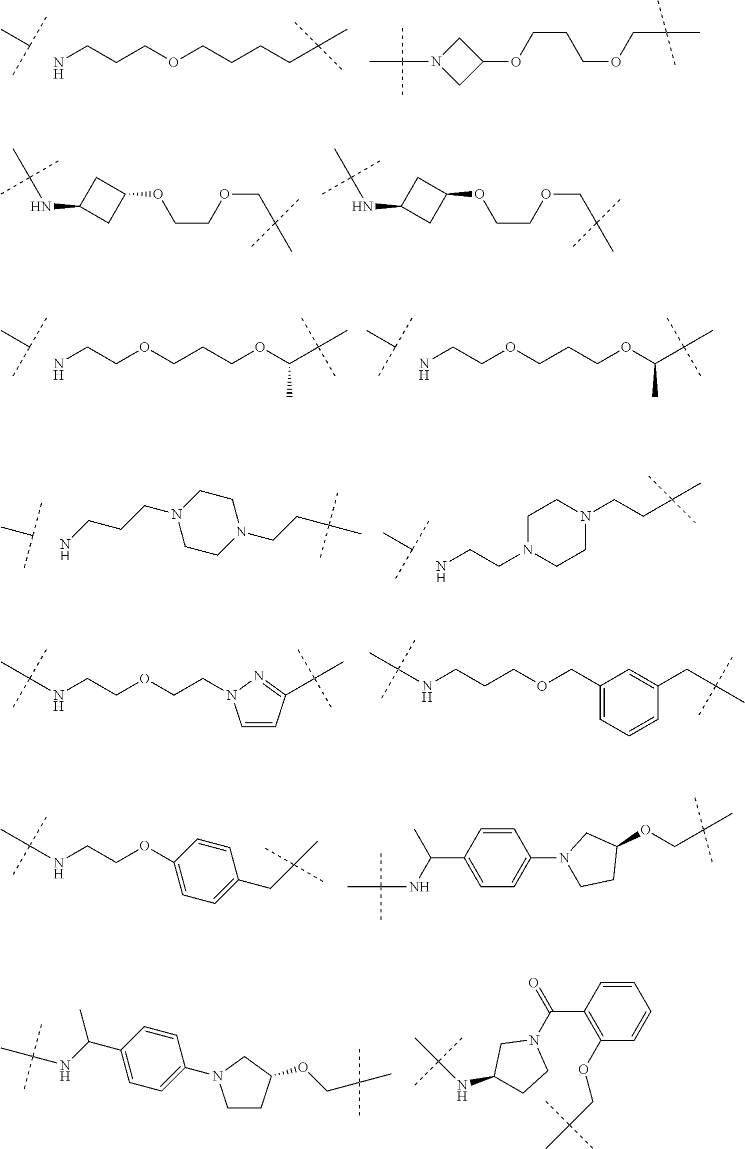

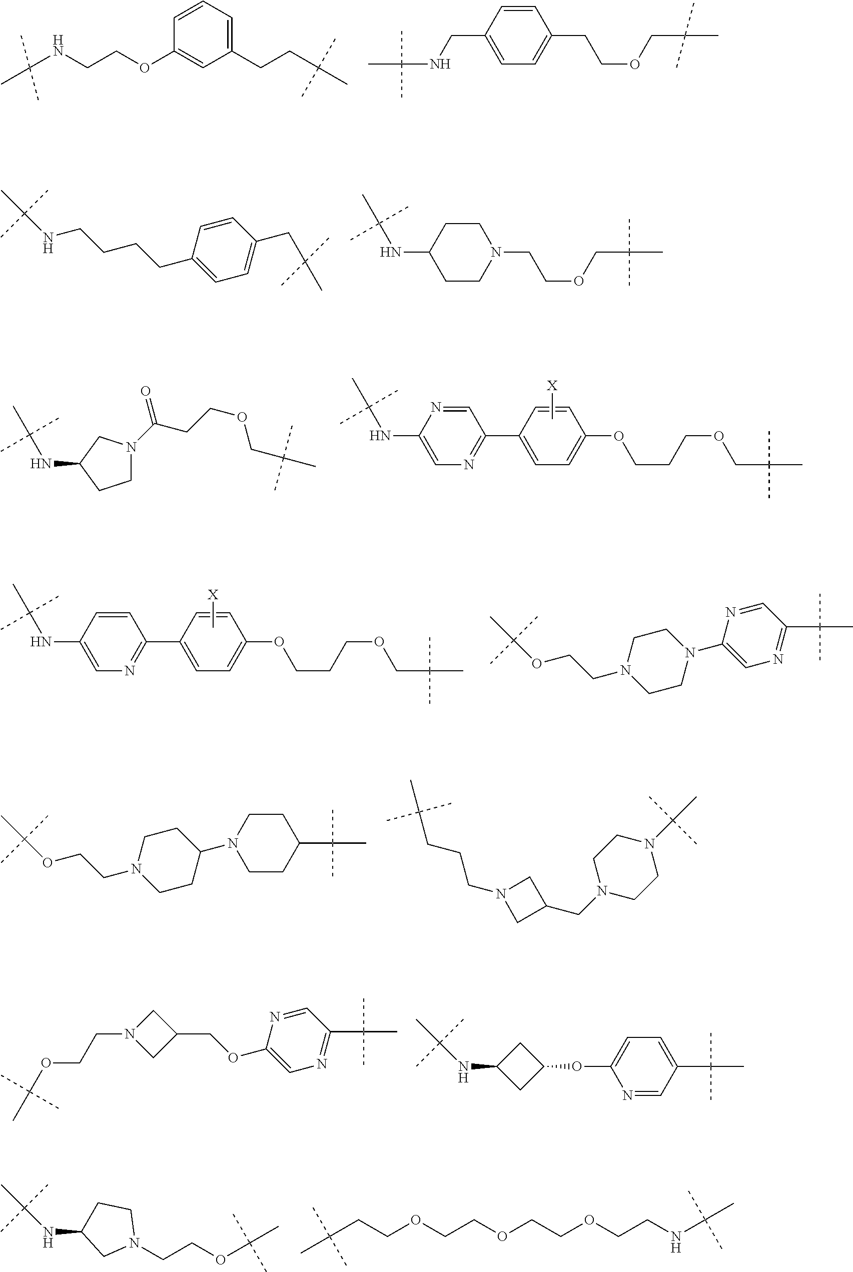

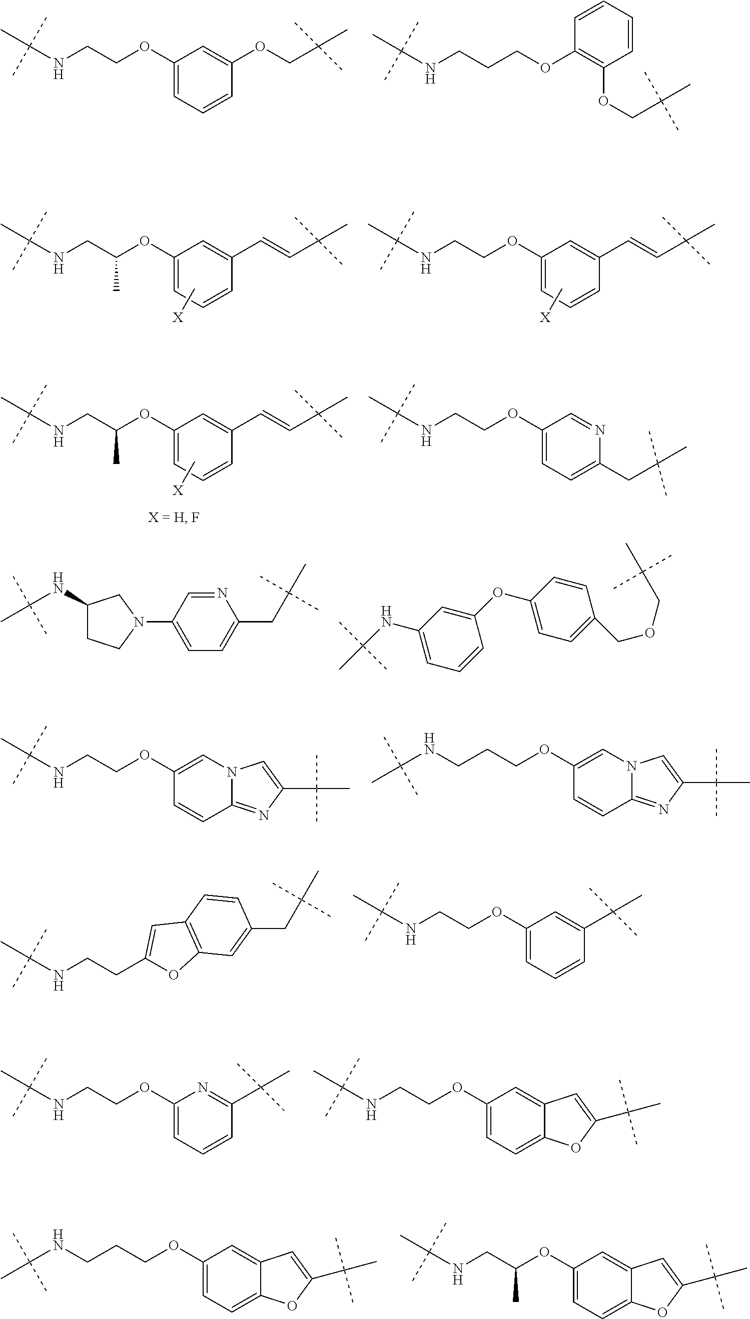

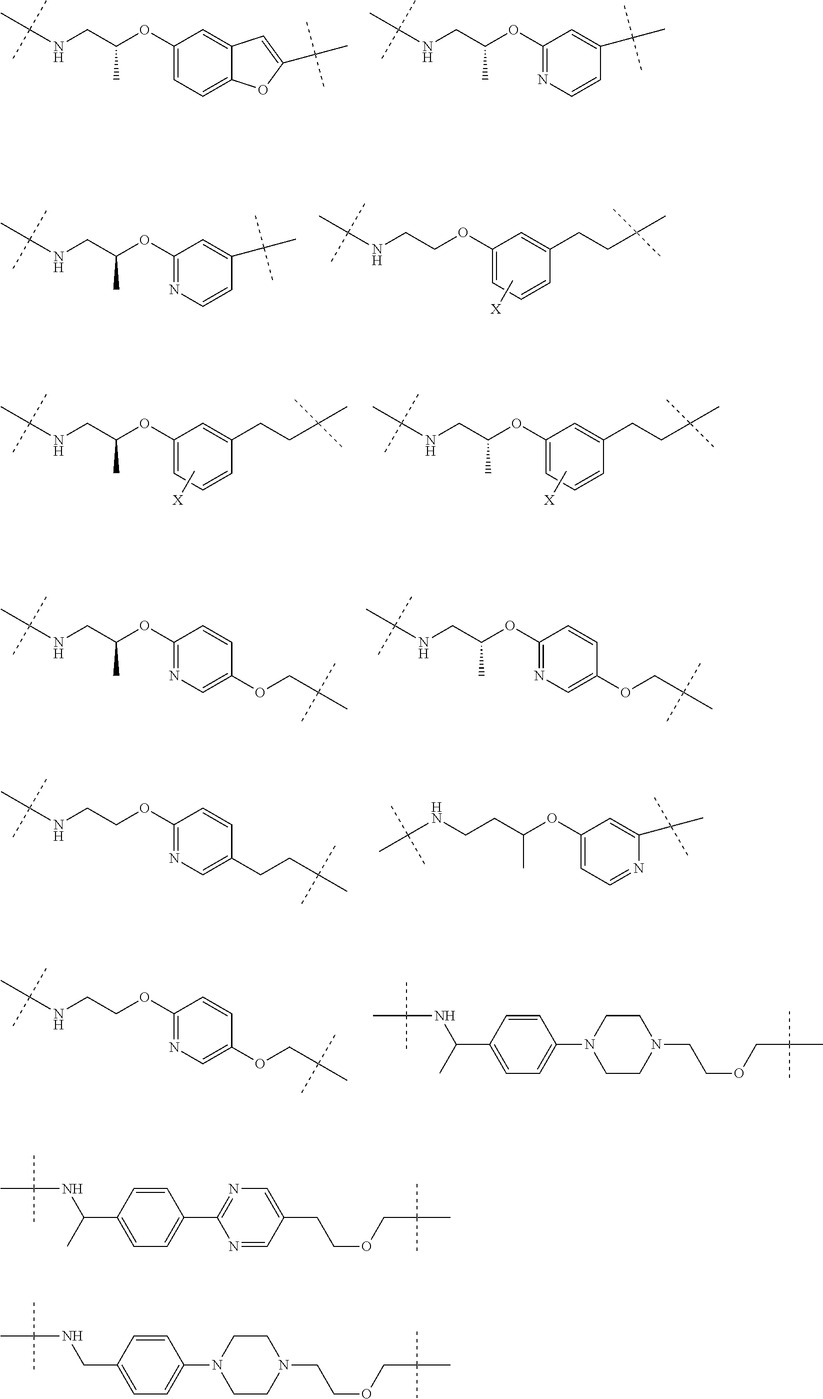

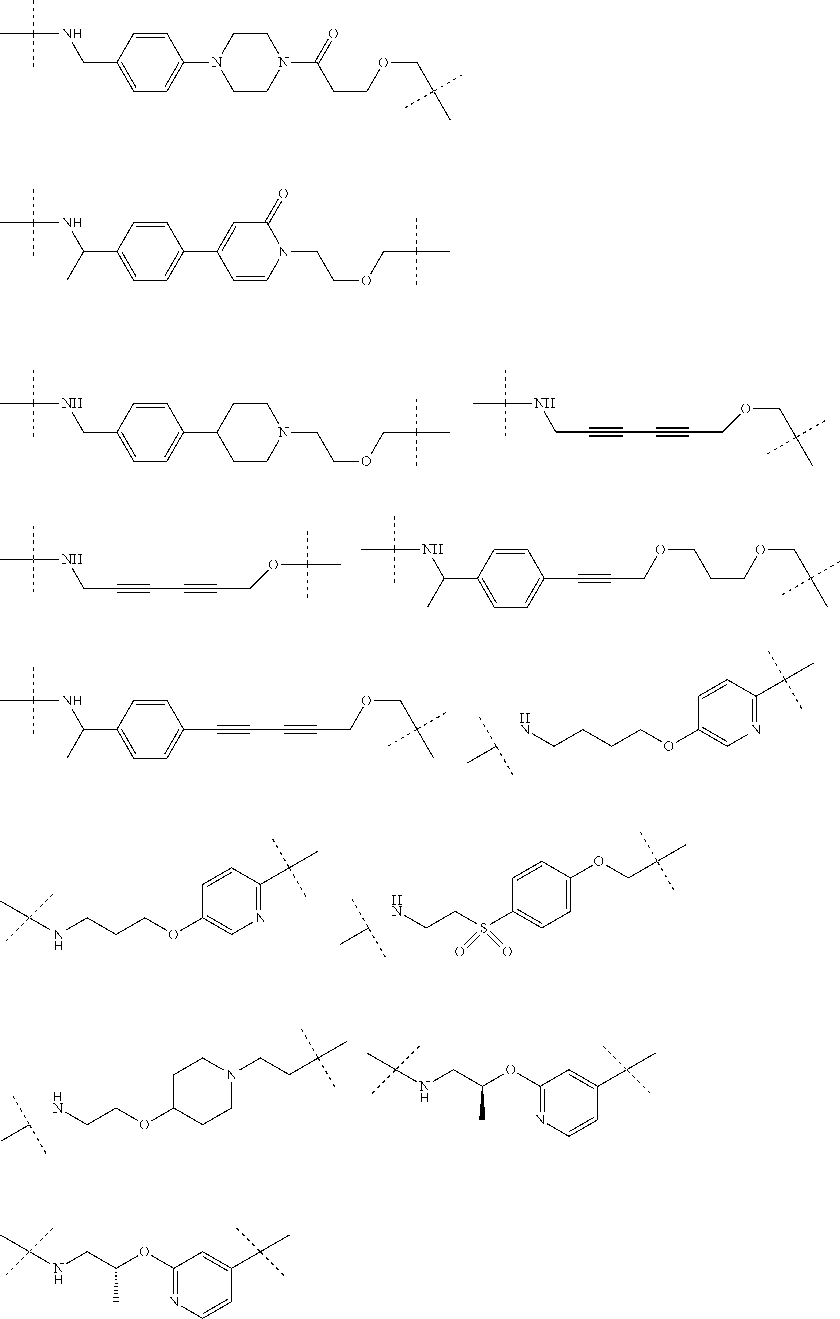

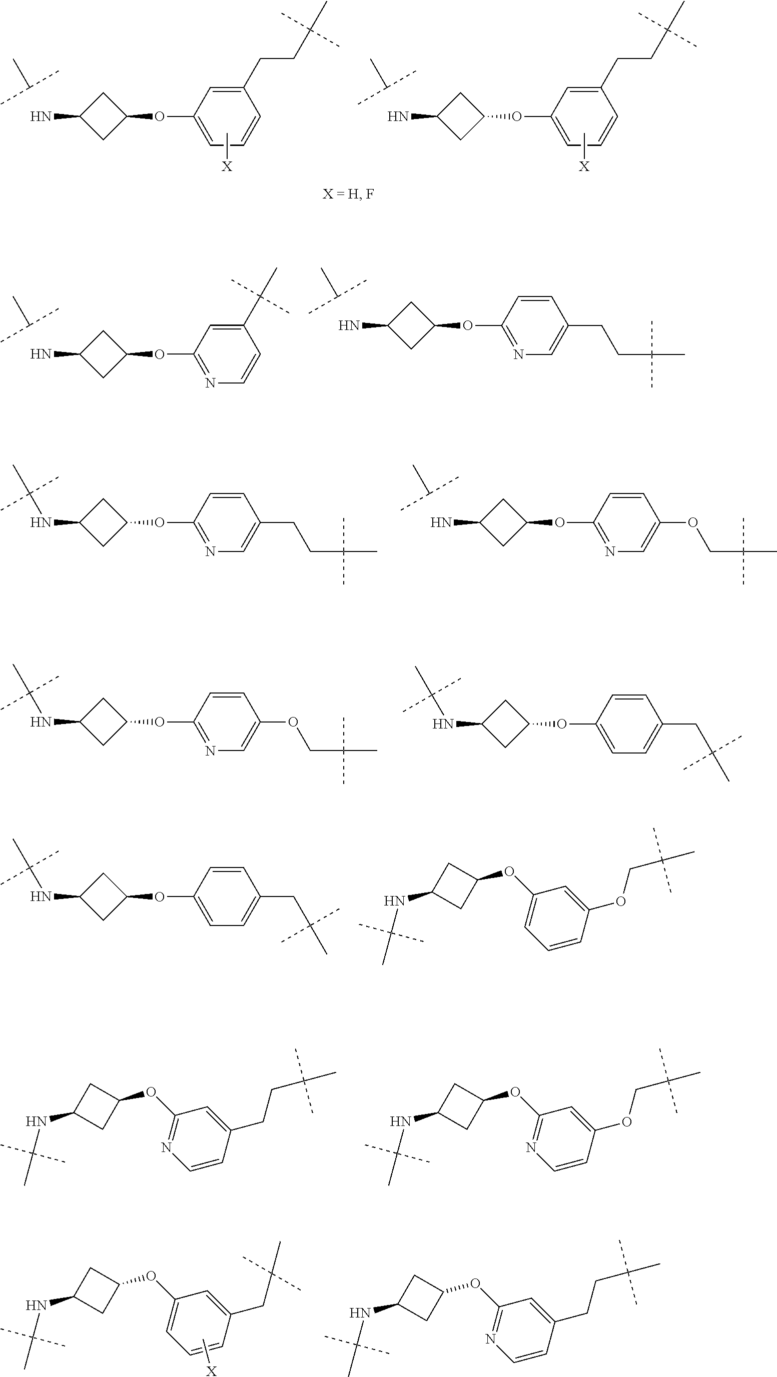

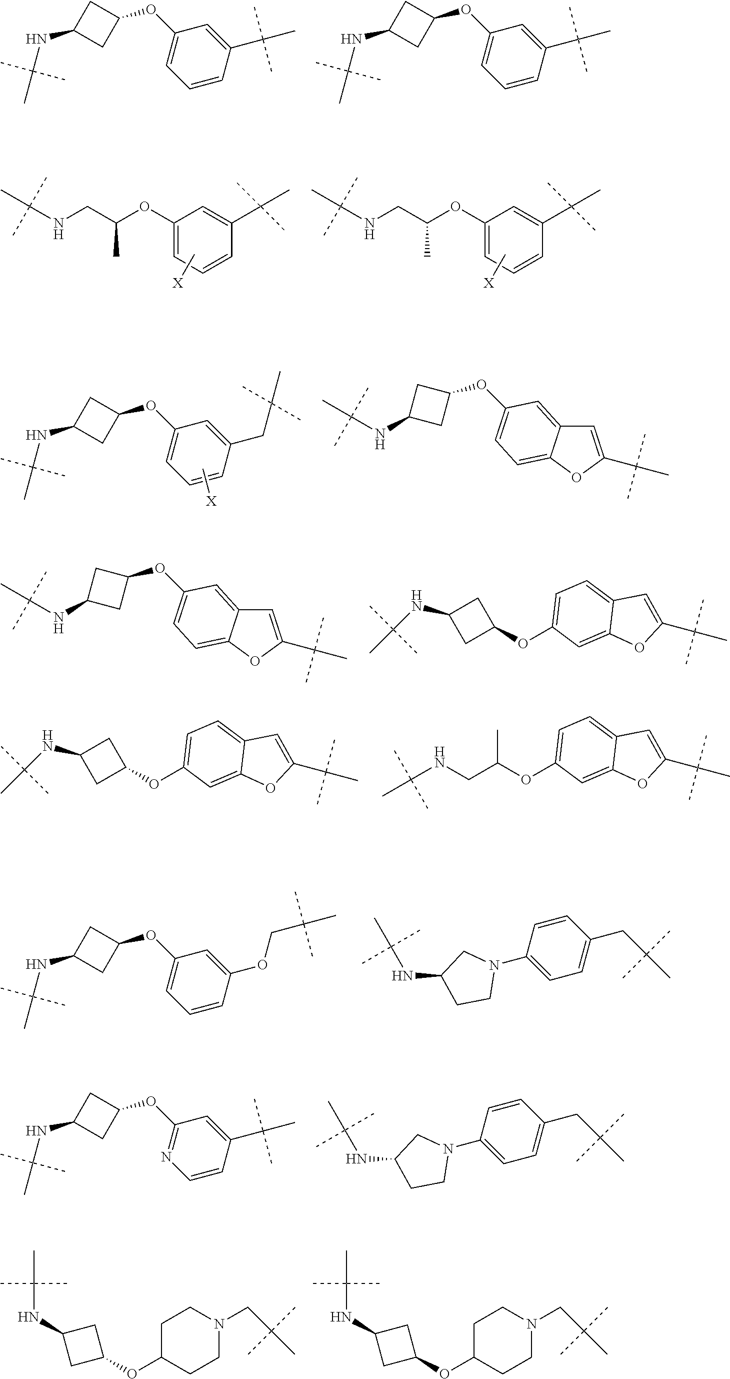

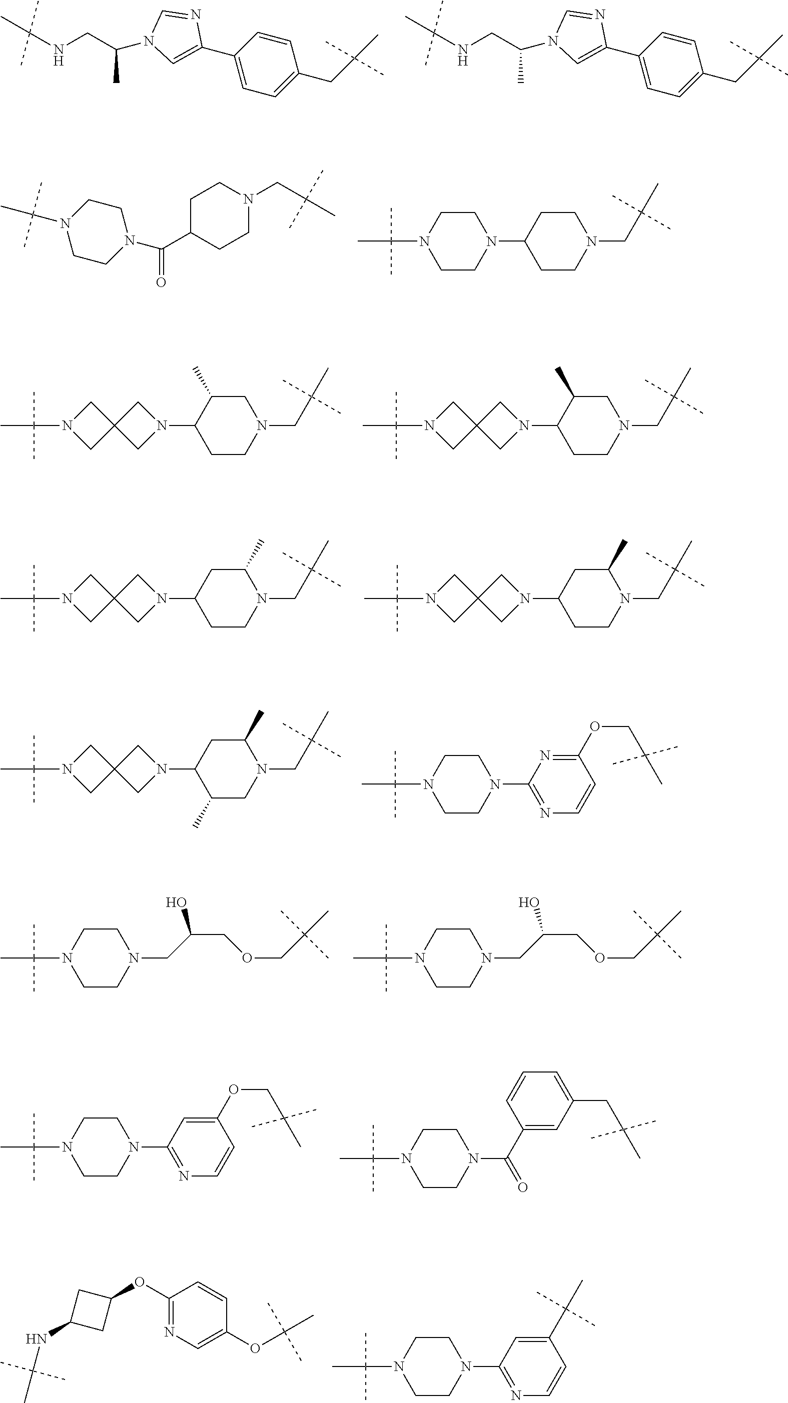

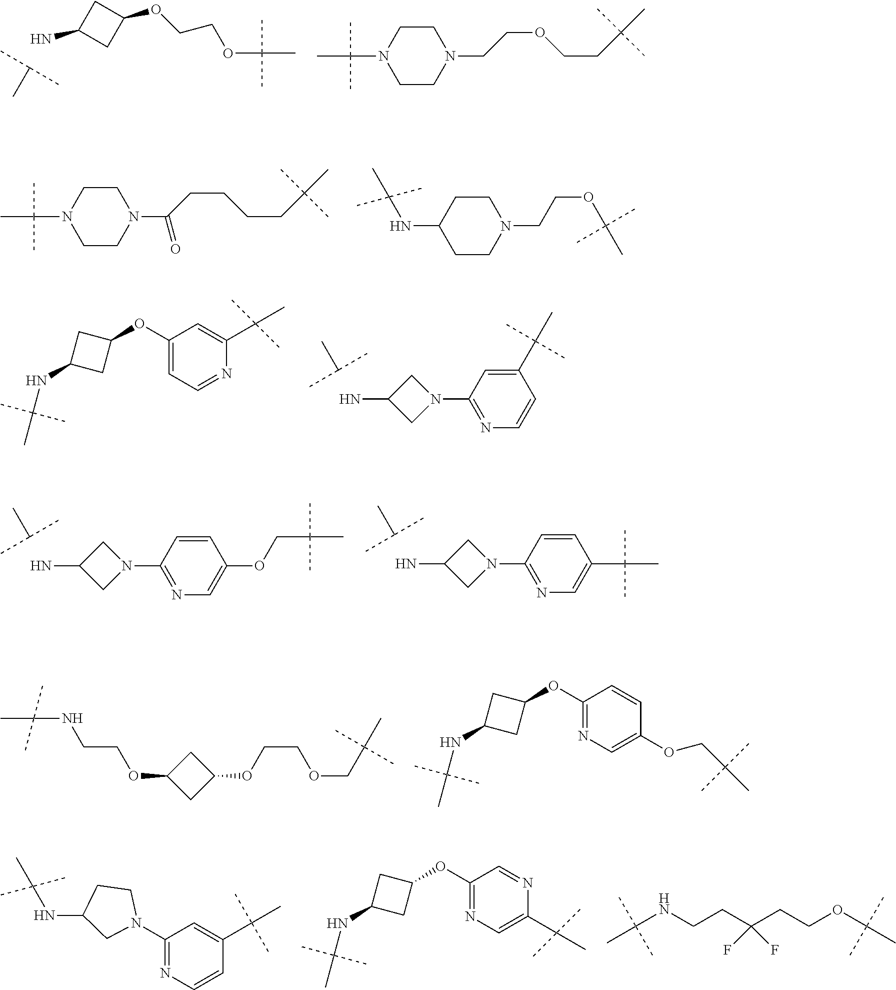

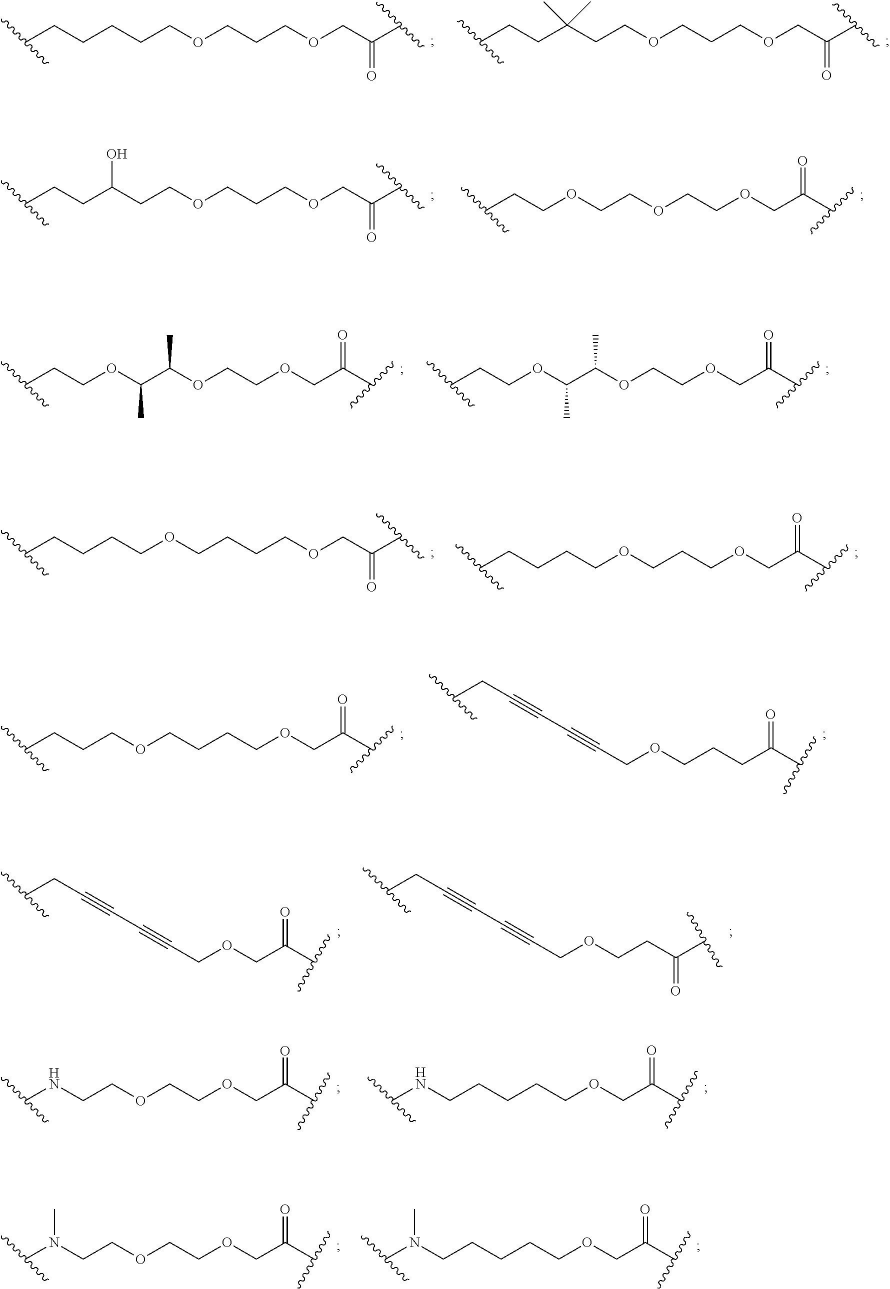

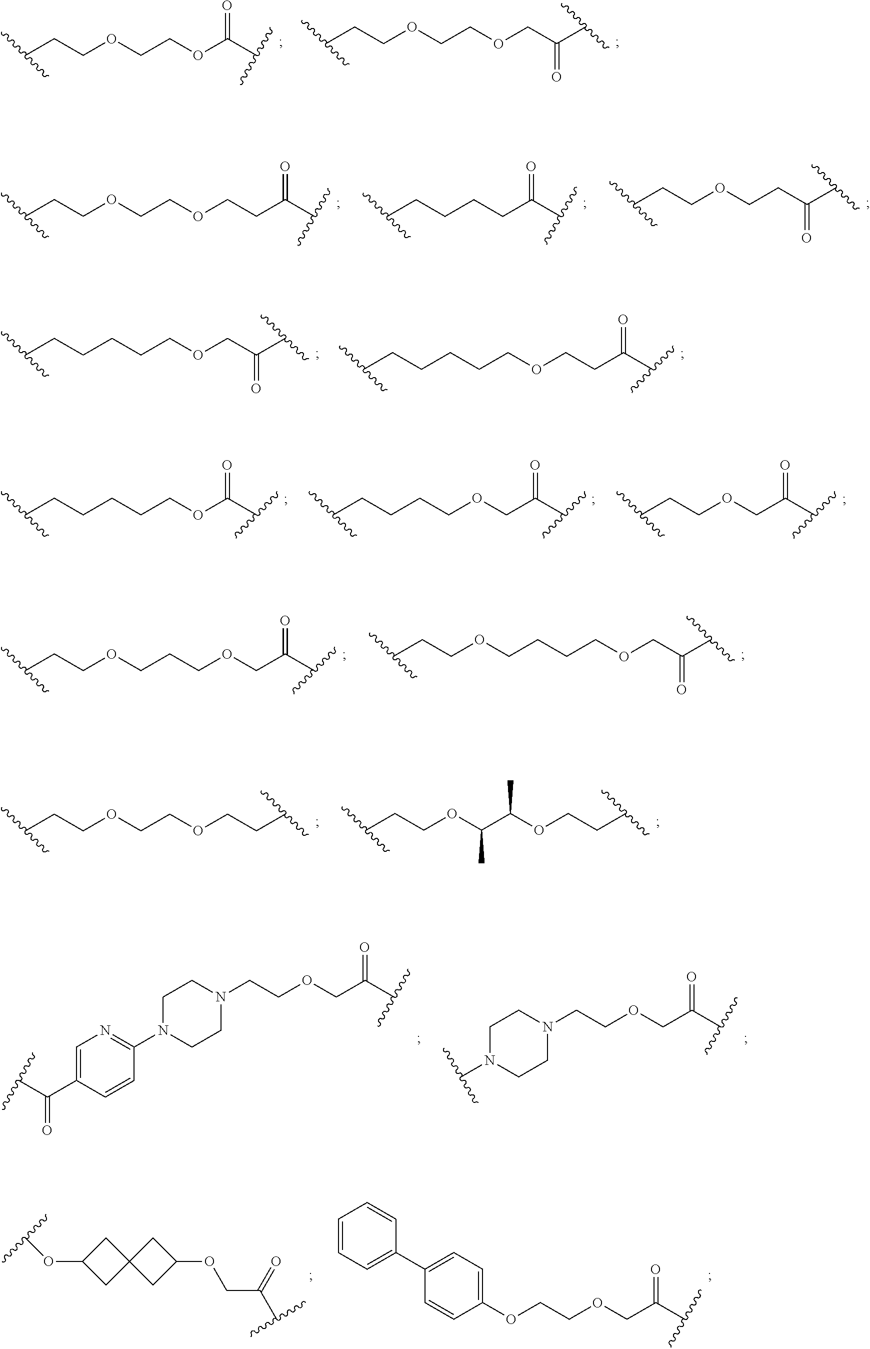

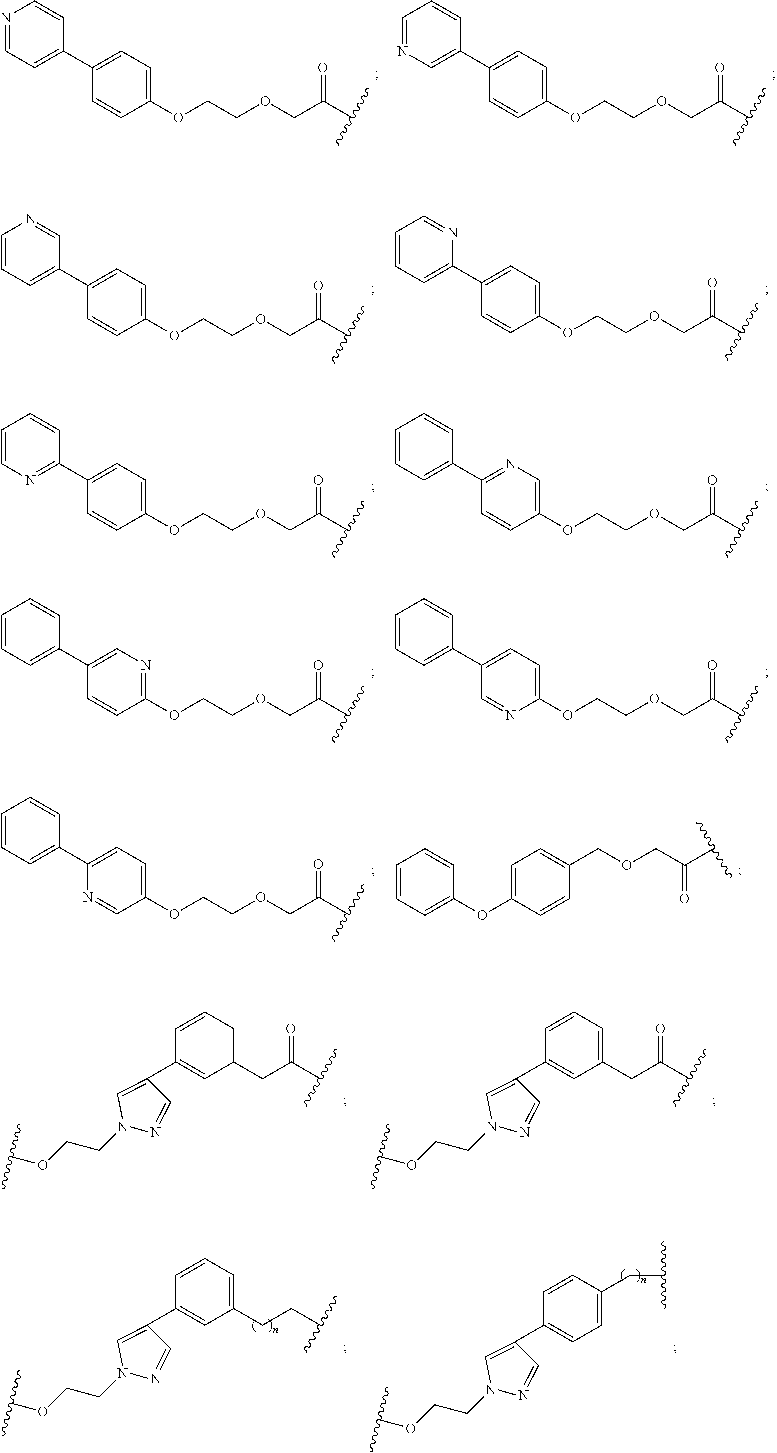

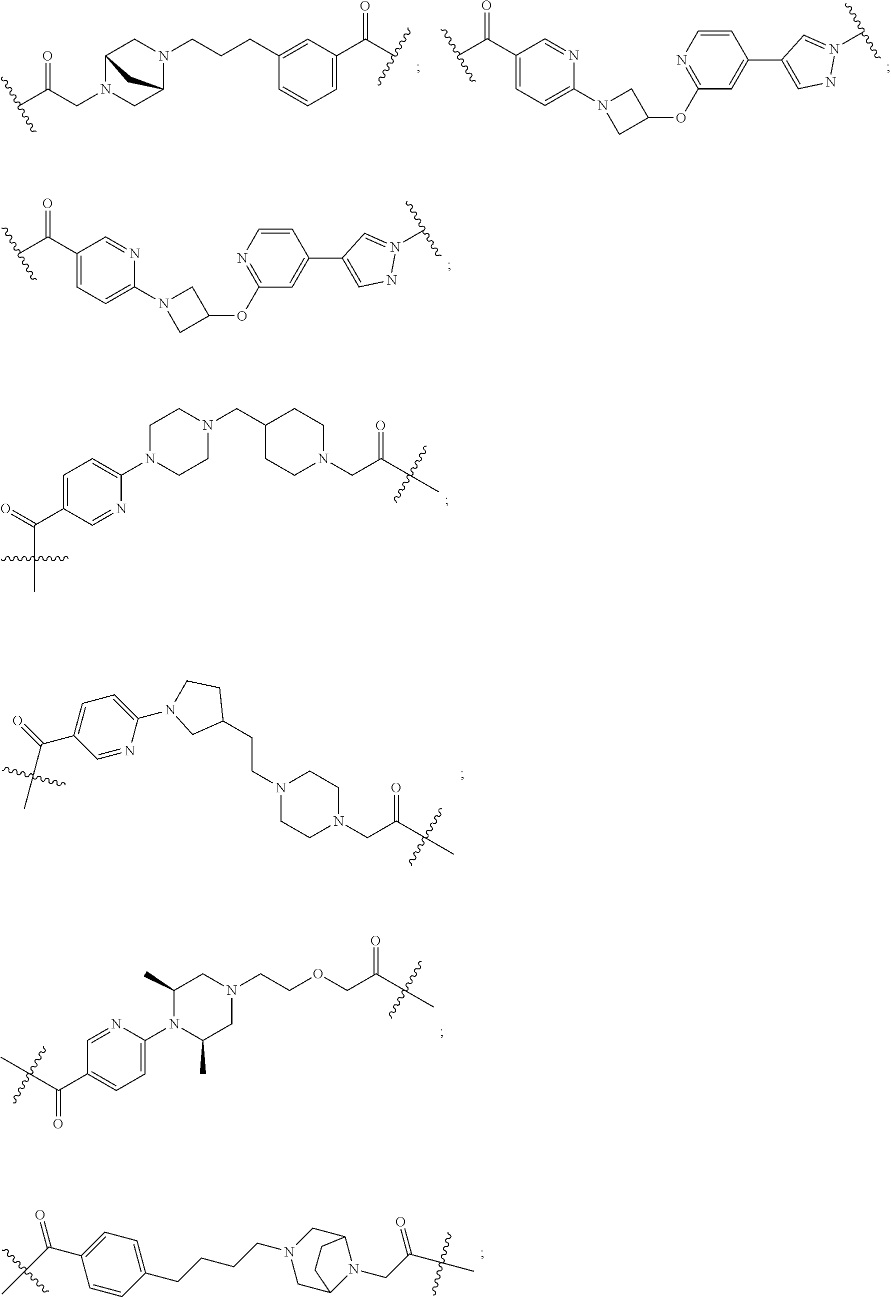

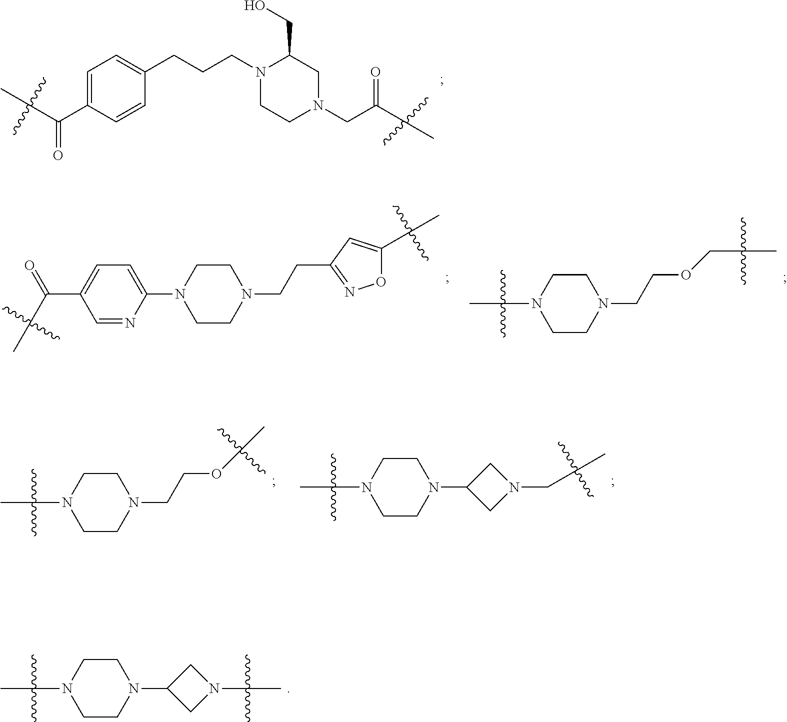 [SMILES: *C(=O)CN1CCN(CCCc2ccc(C(*)=O)cc2)[C@@H](CO)C1 ; *C(=O)c1ccc(N2CCN(CCc3cc(*)on3)CC2)nc1 ; *COCCN1CCN(*)CC1 ; *OCCN1CCN(*)CC1 ; *CN1CC(N2CCN(*)CC2)C1 ; *N1CCN(C2CN(*)C2)CC1]

In any aspect or embodiment described herein, the unit A^{L} of linker (L) is selected from the group consisting of:

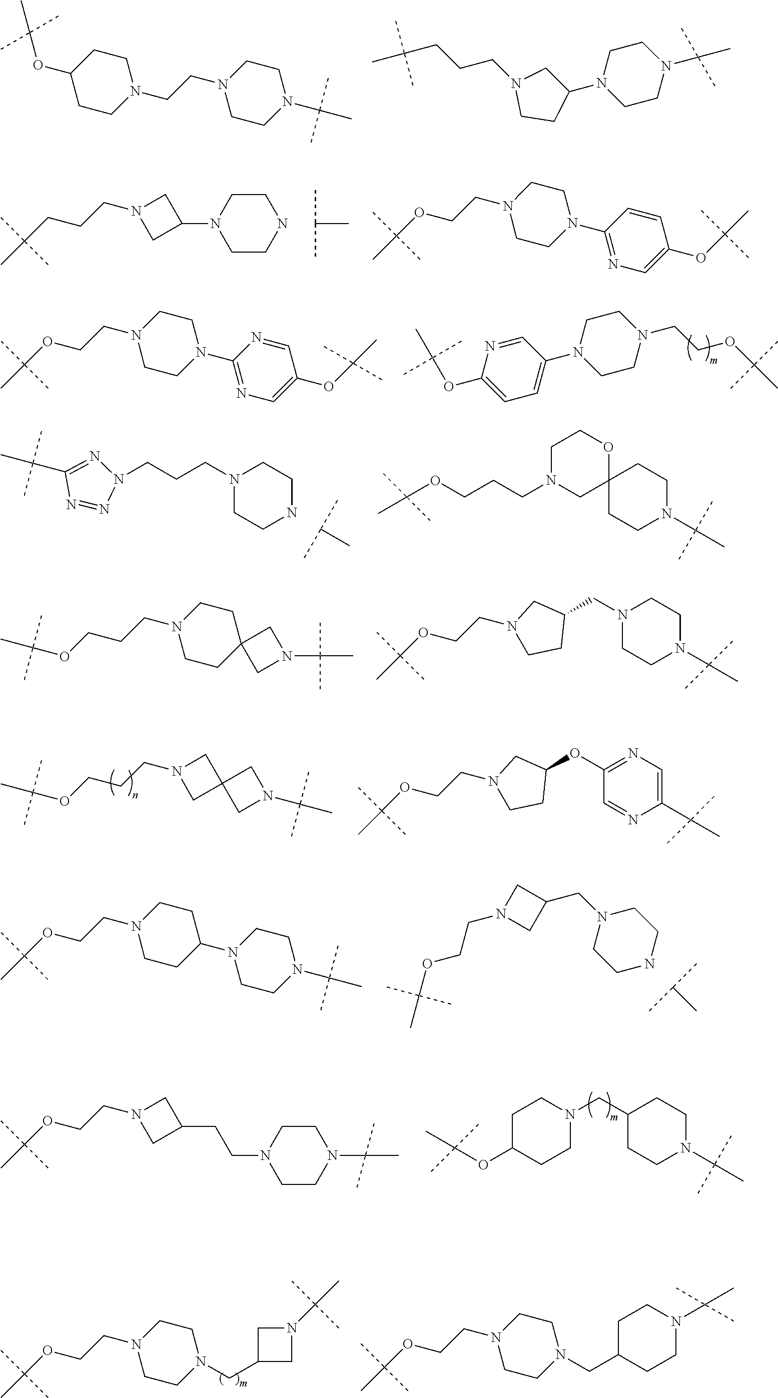

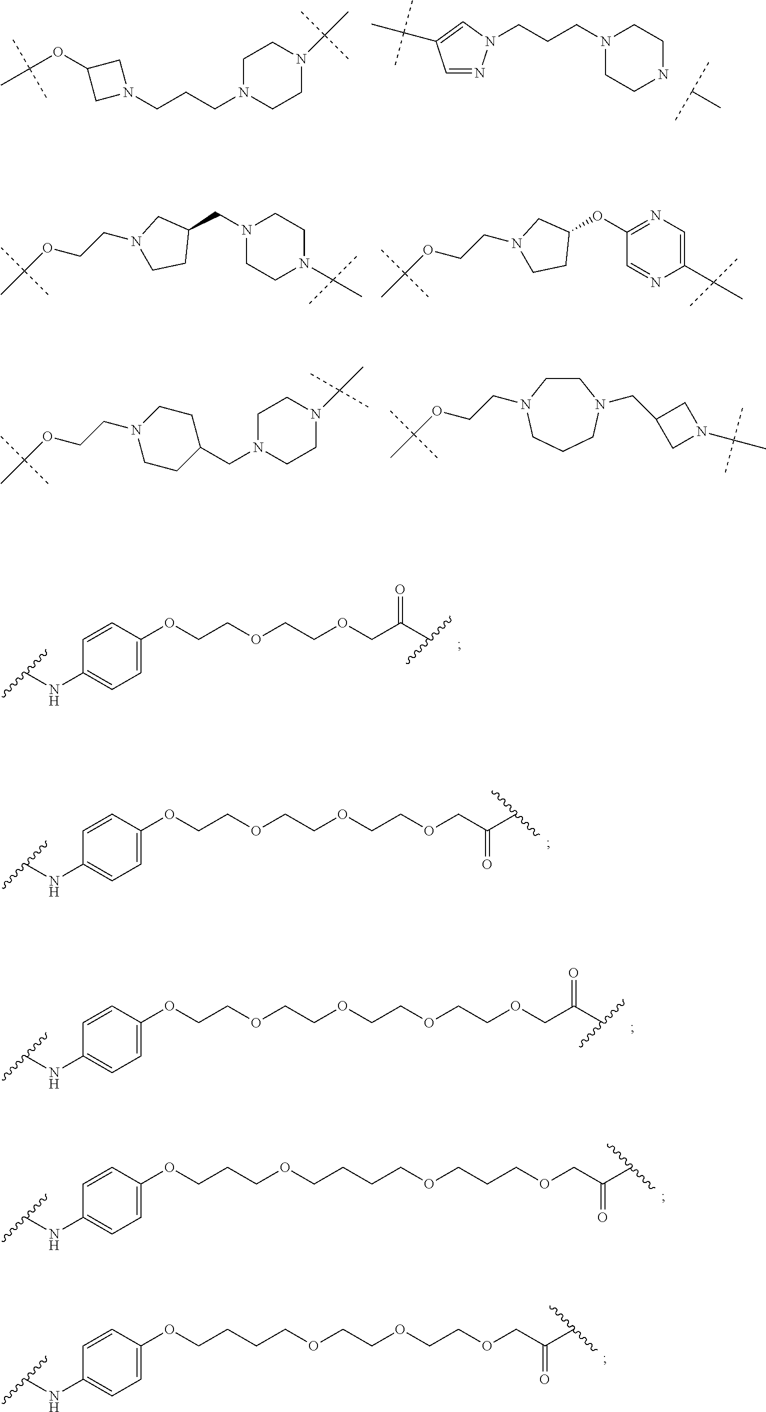

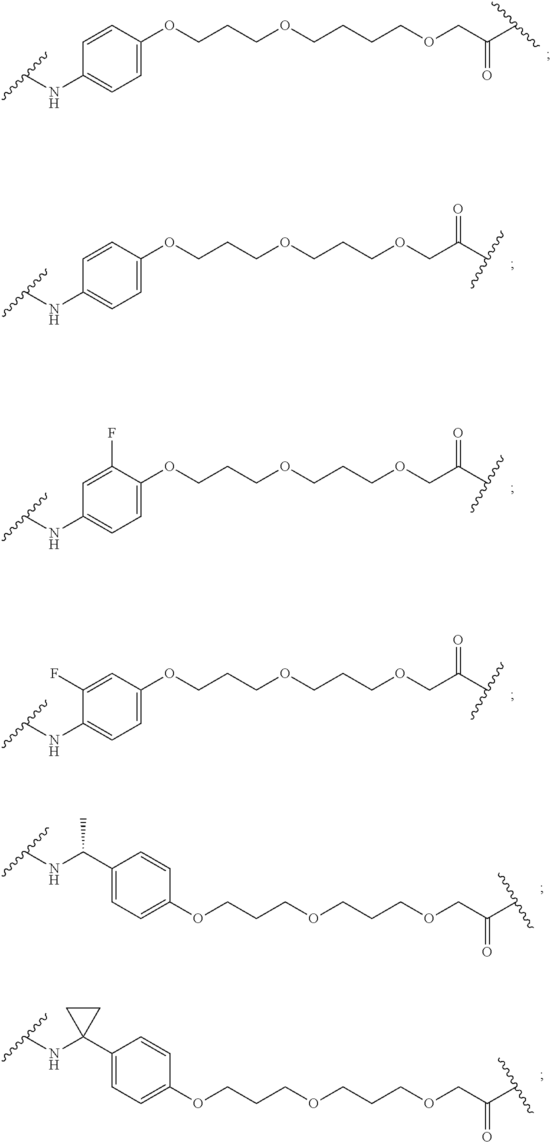

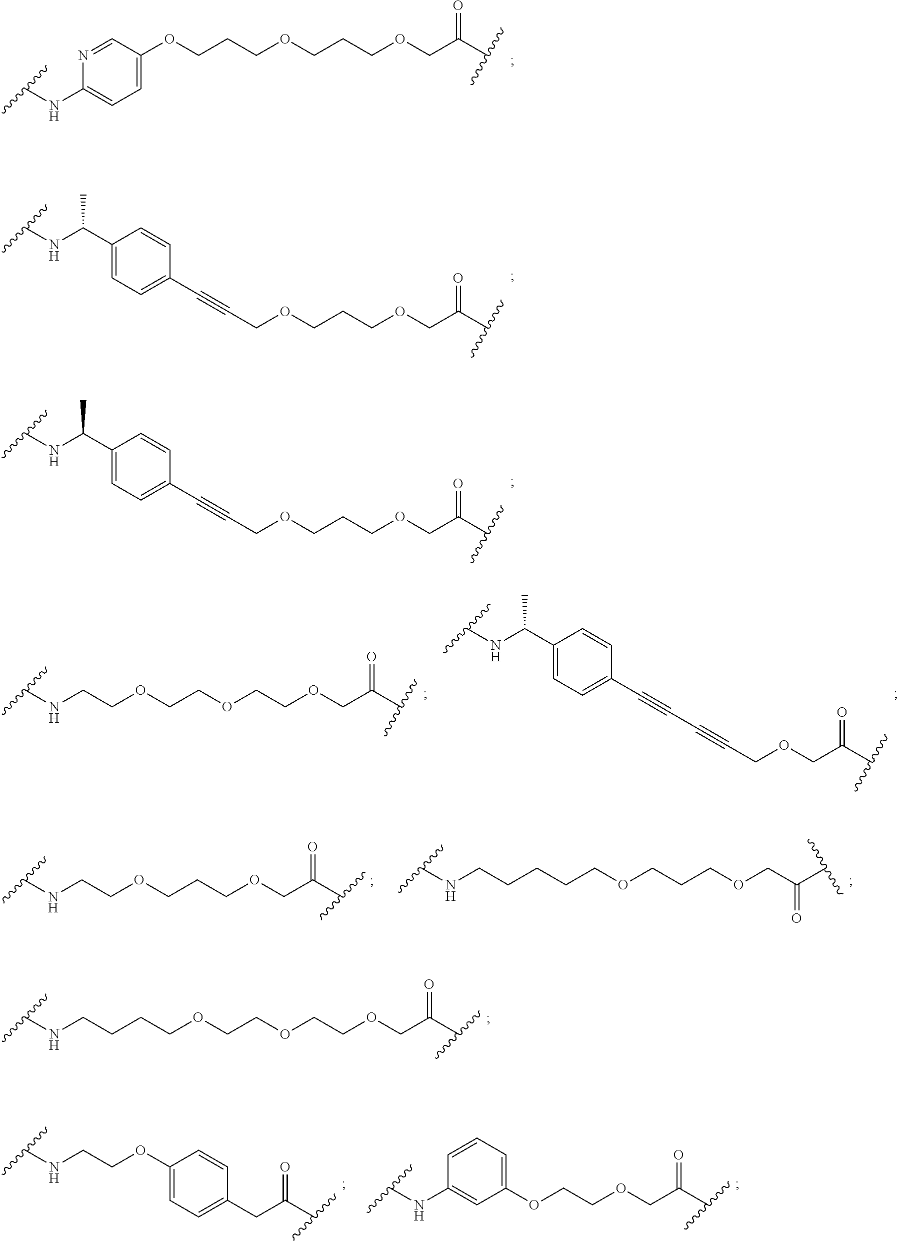

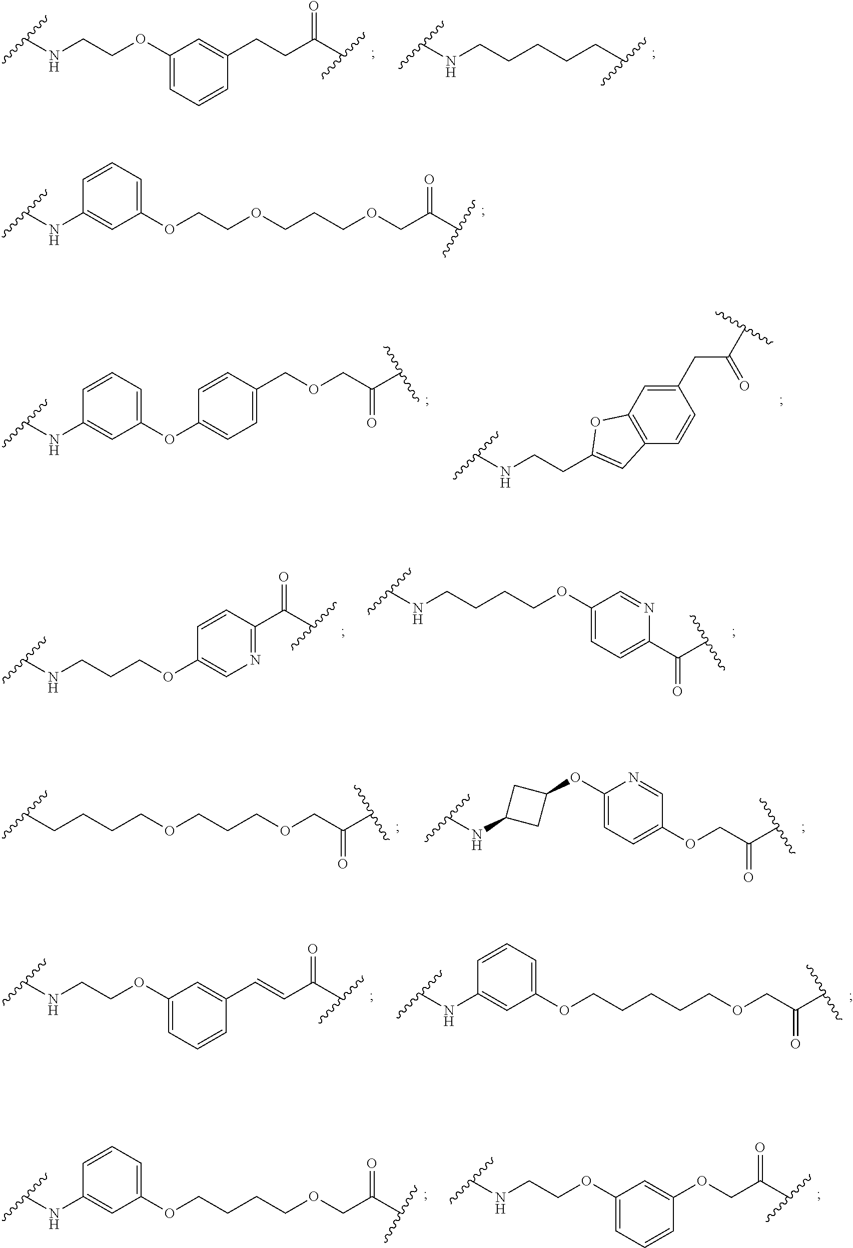

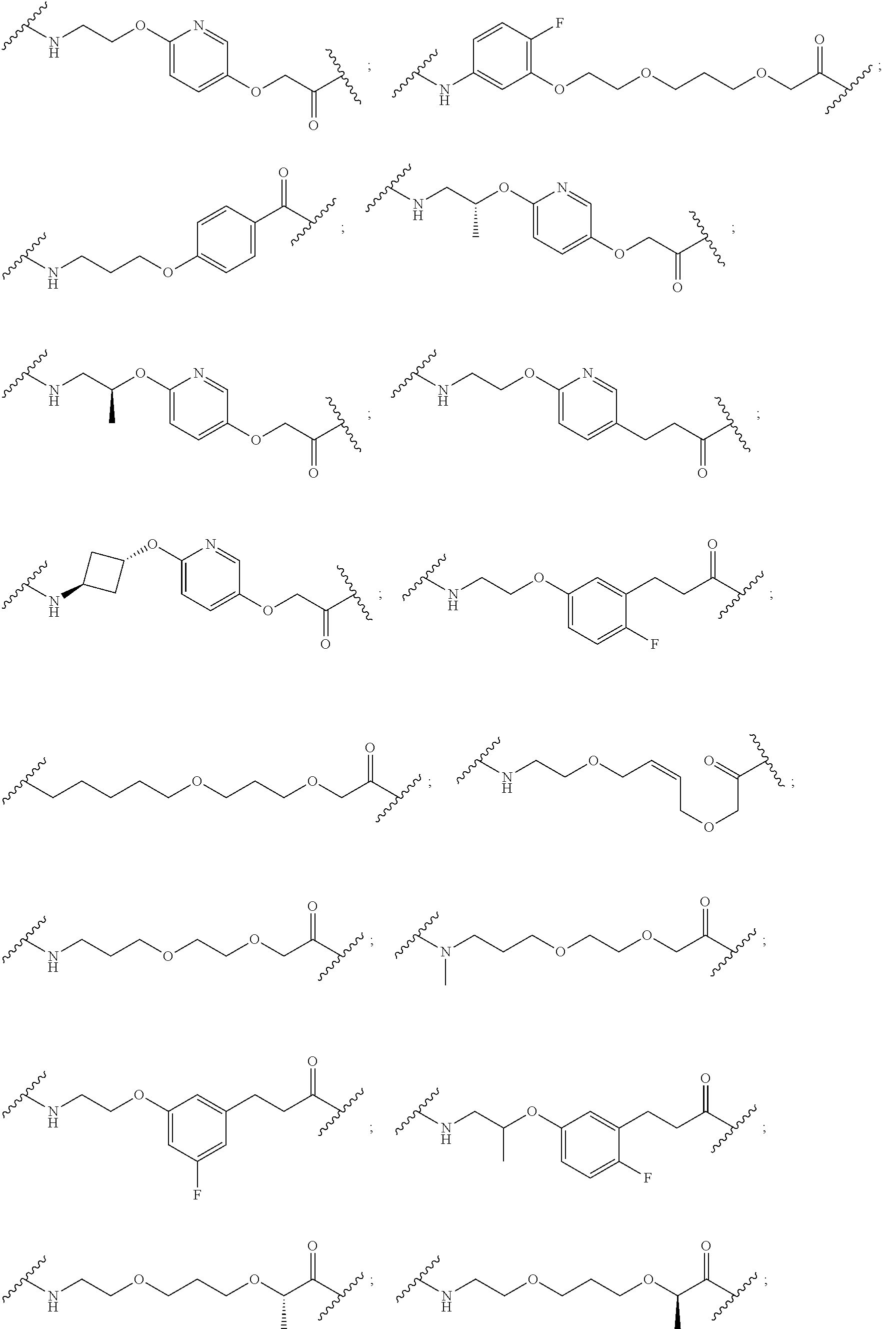

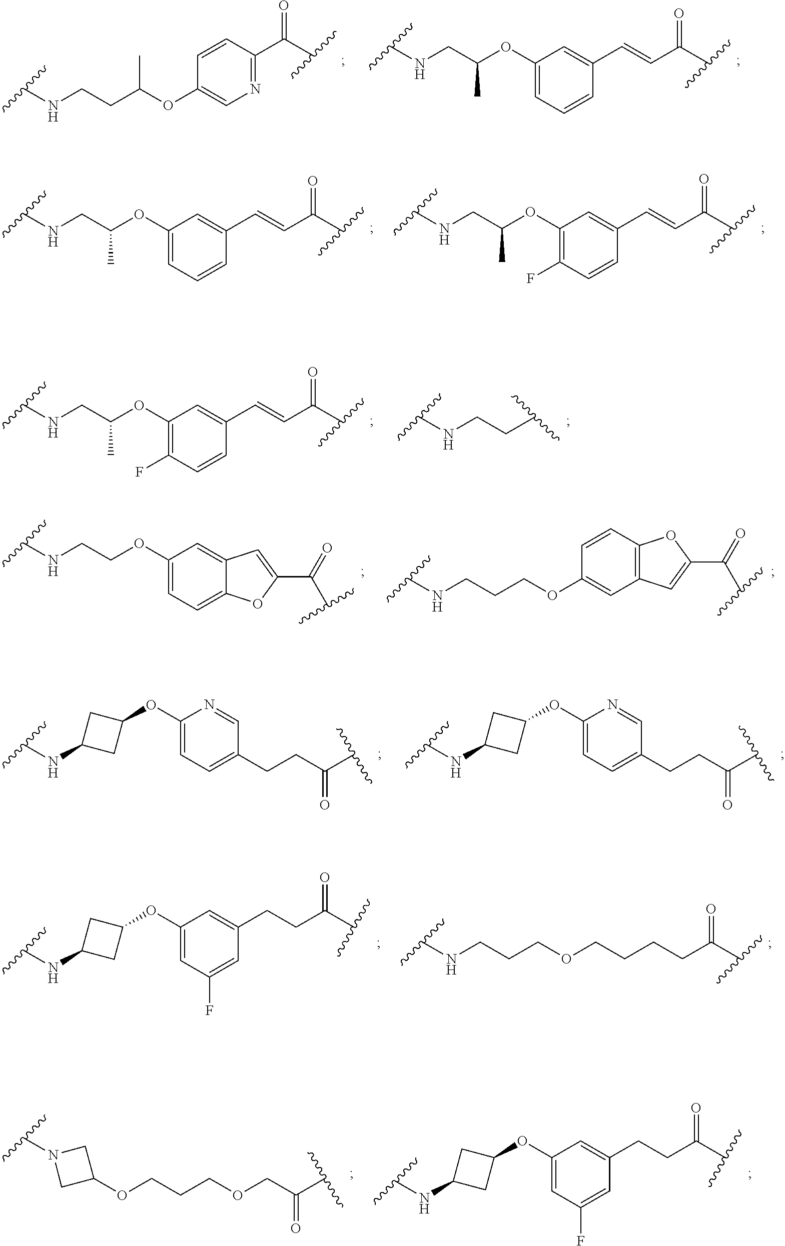

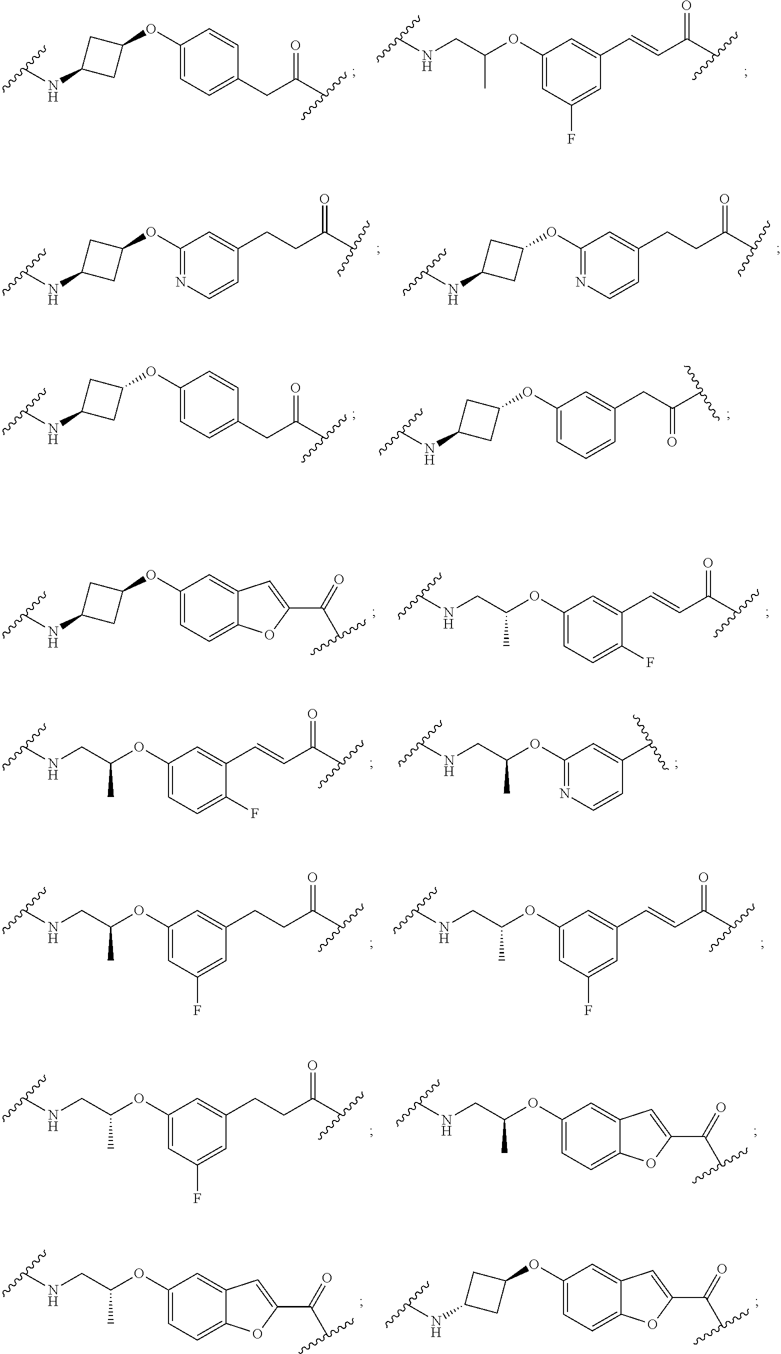

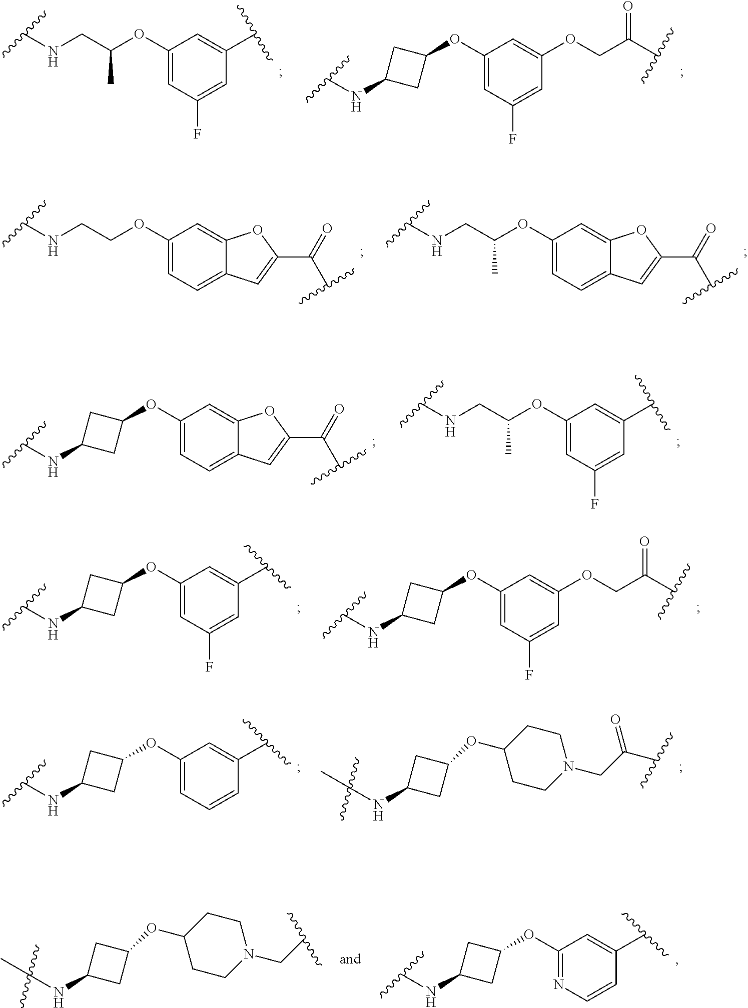

wherein each m and n is independently selected from 0, 1, 2, 3, 4, 5, 6, 7, 8, 9, 10, 11, 12, 13, 14, 15, 16, 17, 18, 19, or 20.

In any aspect or embodiment described herein, the unit A^{L} of linker (L) is selected from the group consisting of:

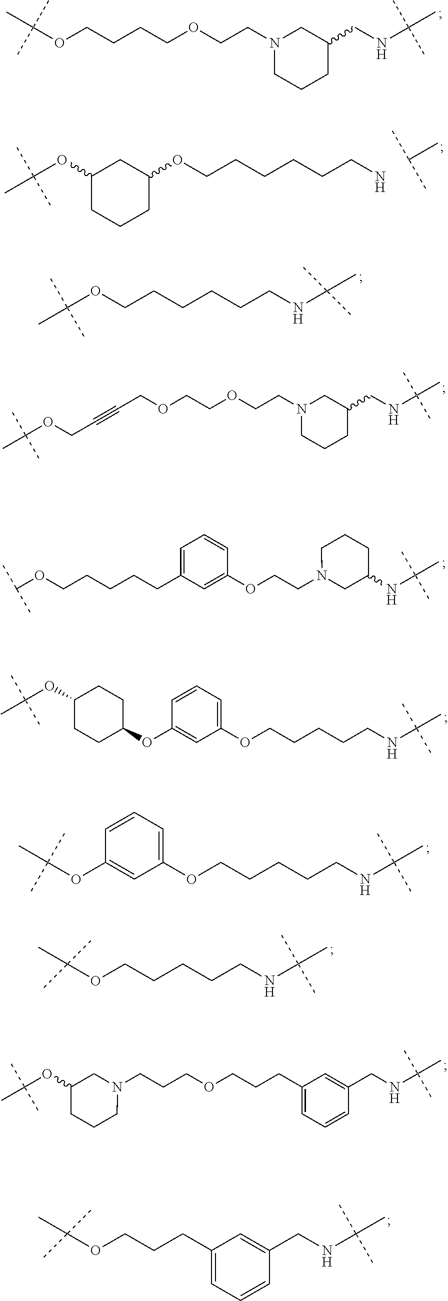

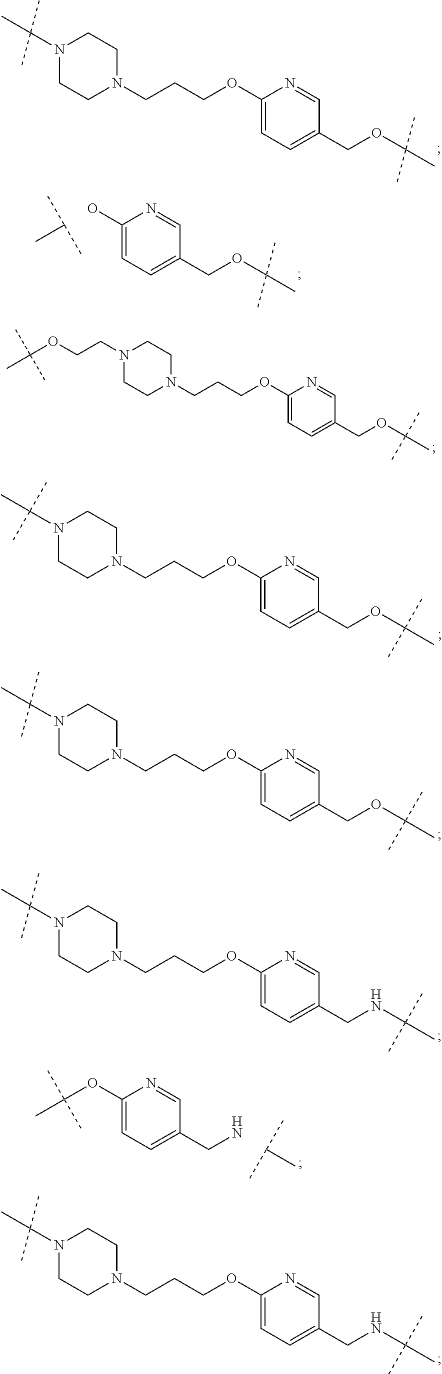

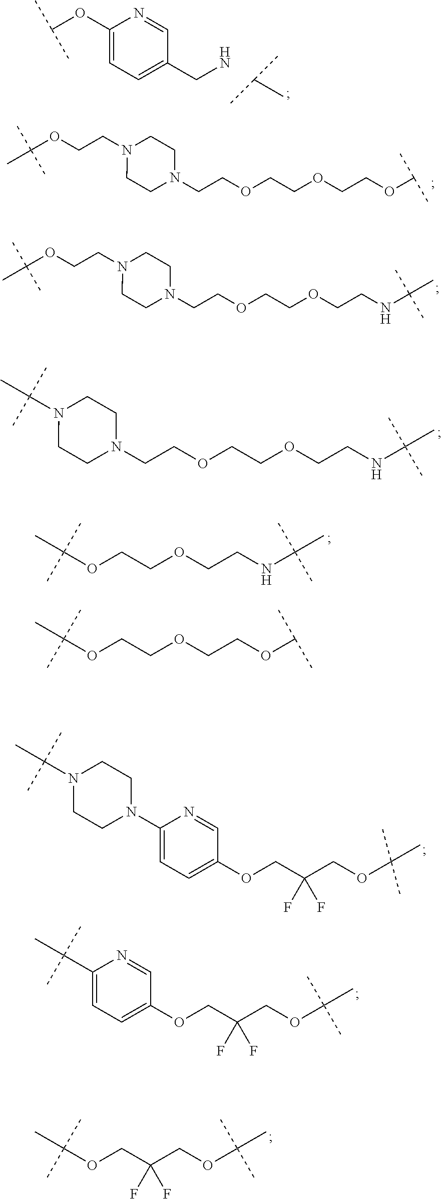

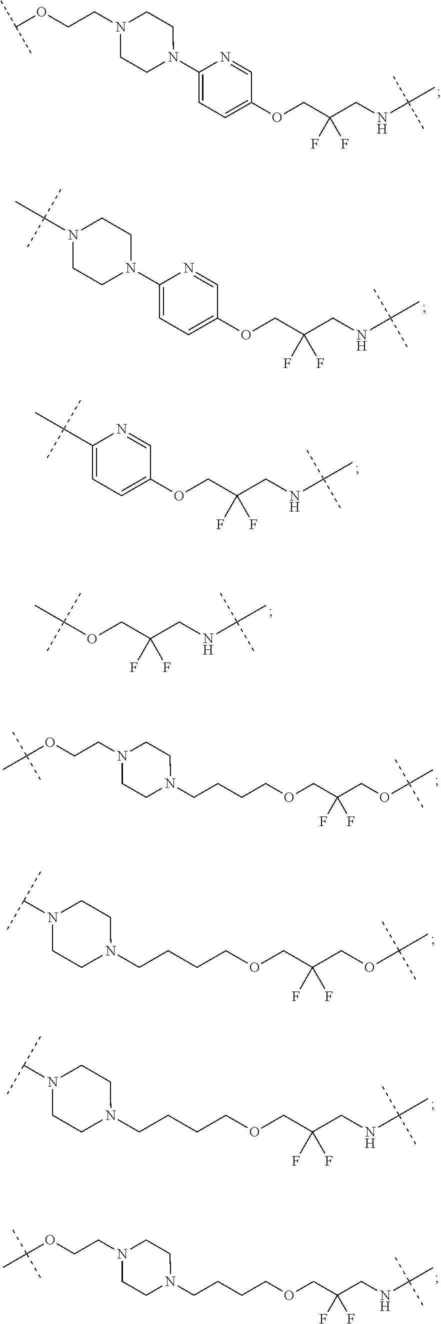

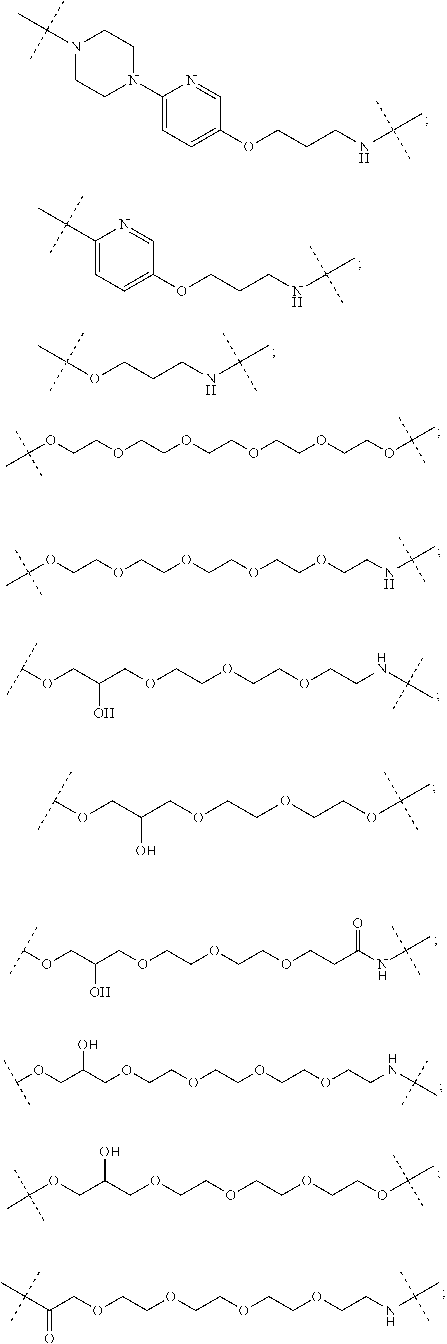

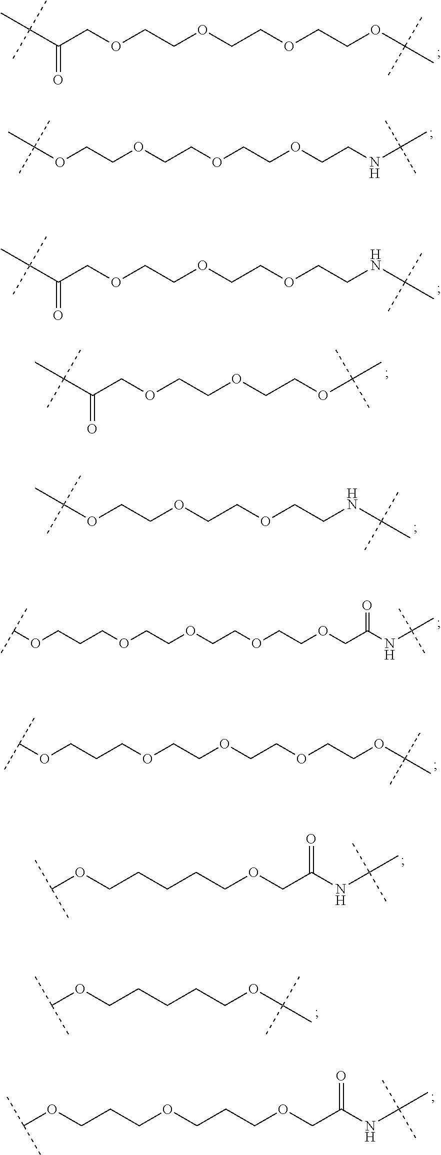

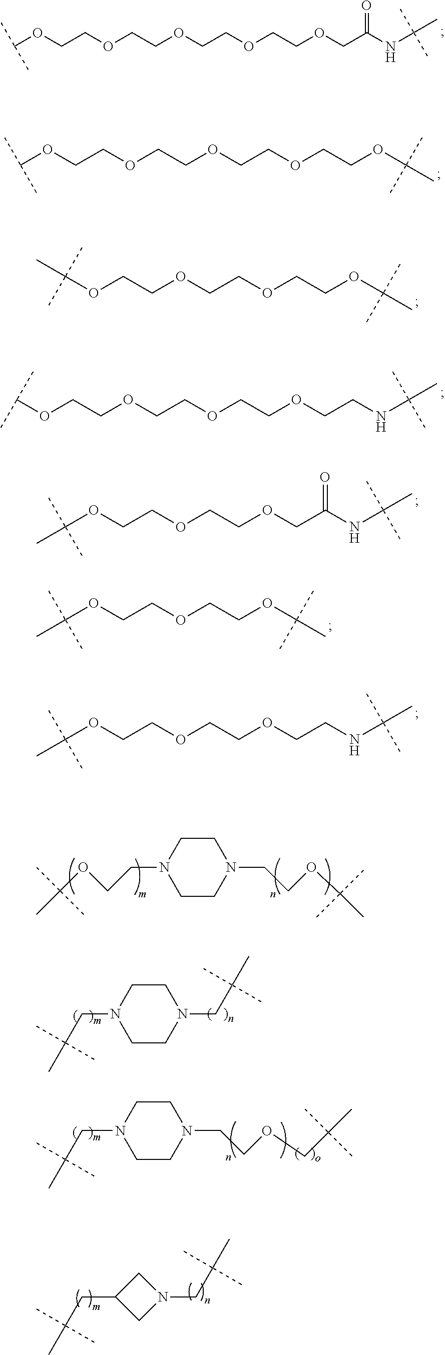

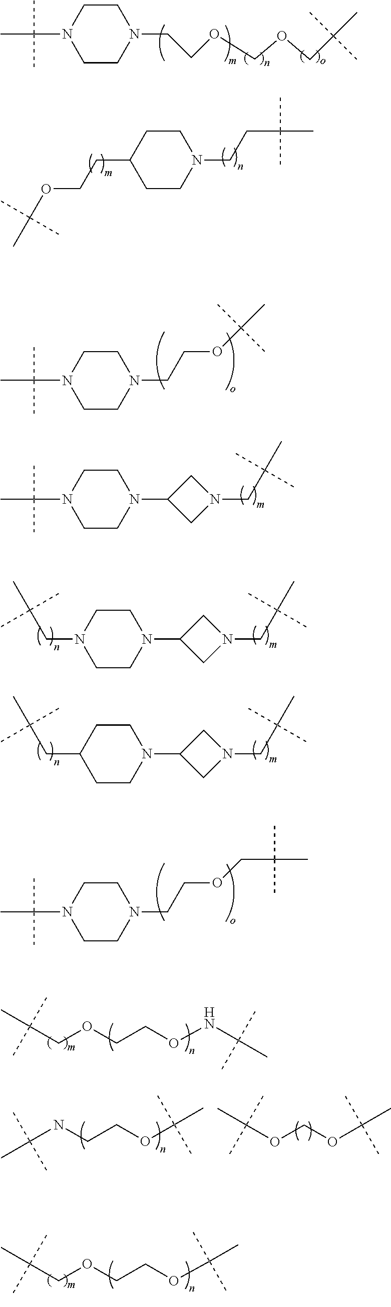

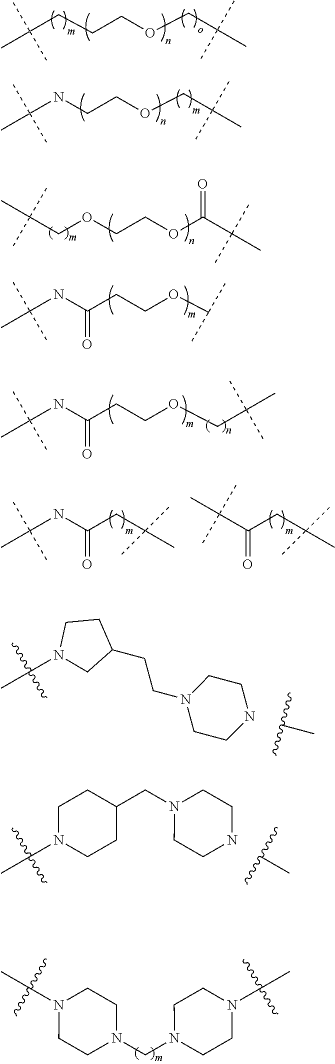

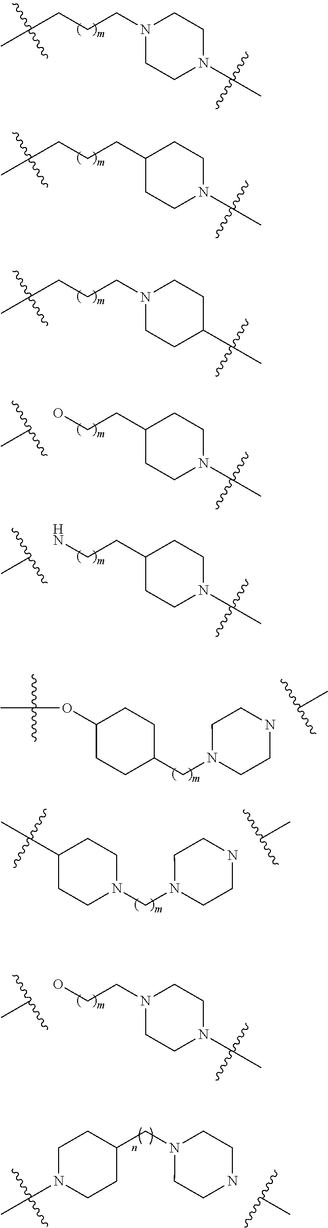

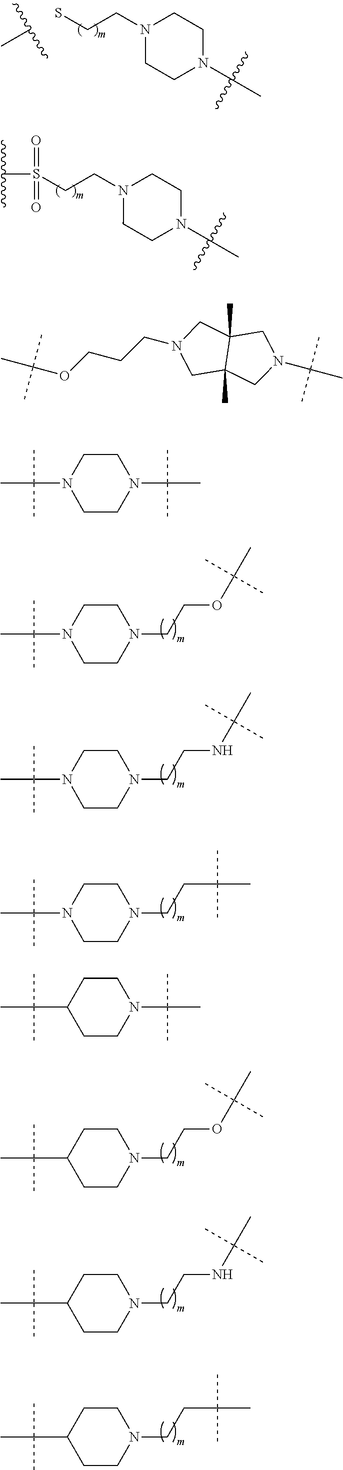

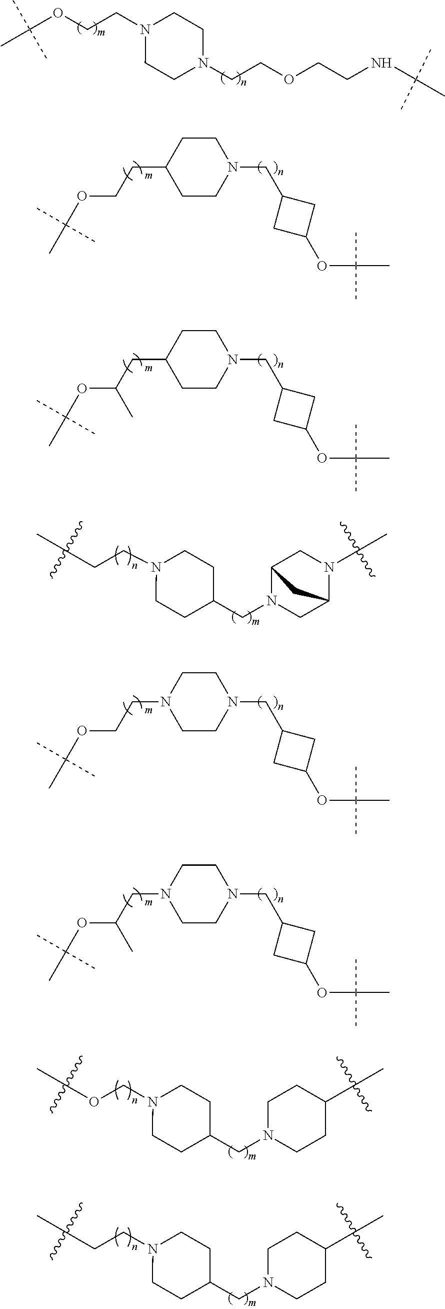

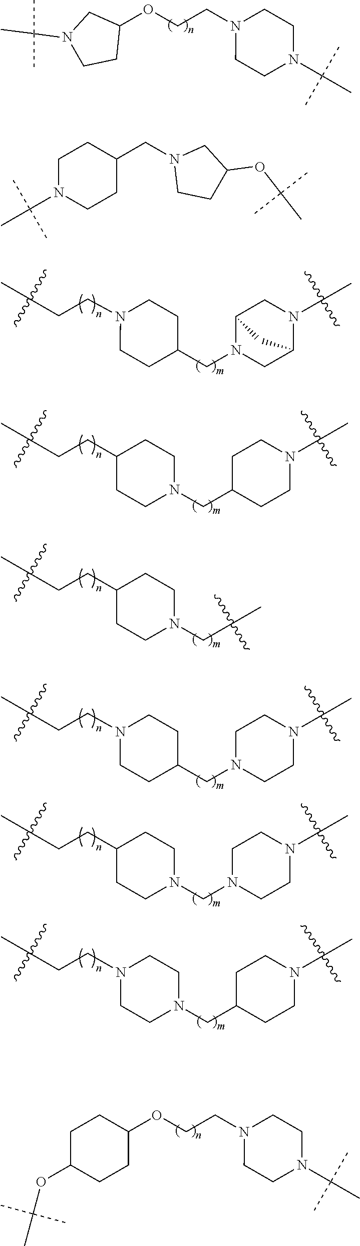

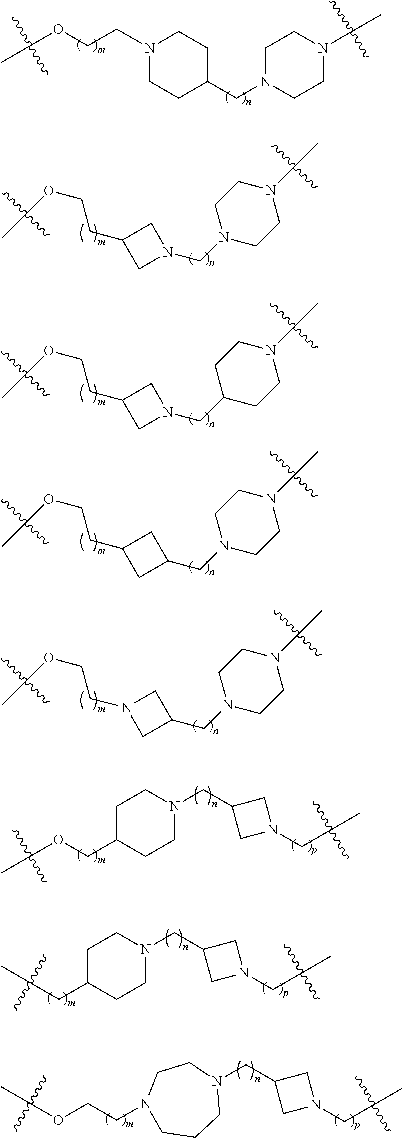

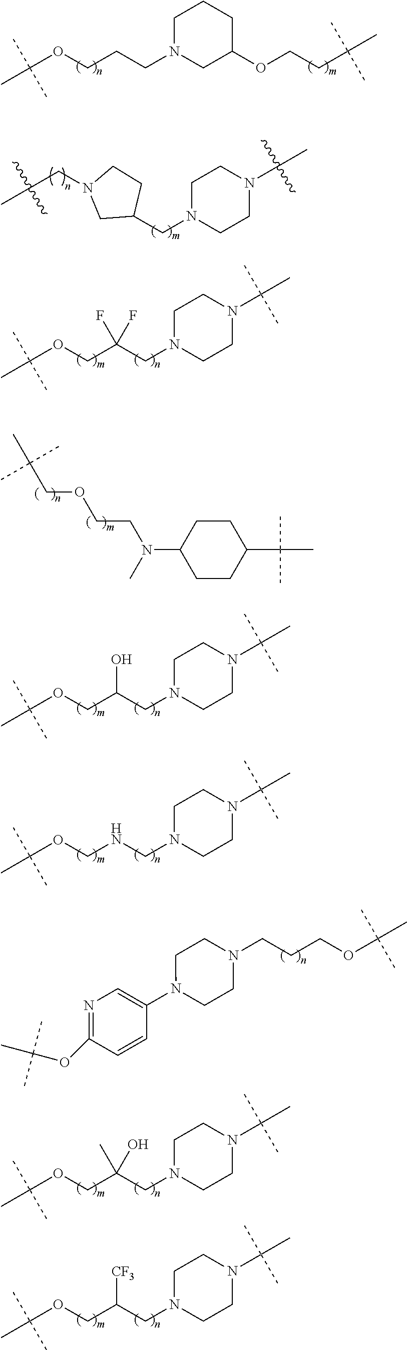

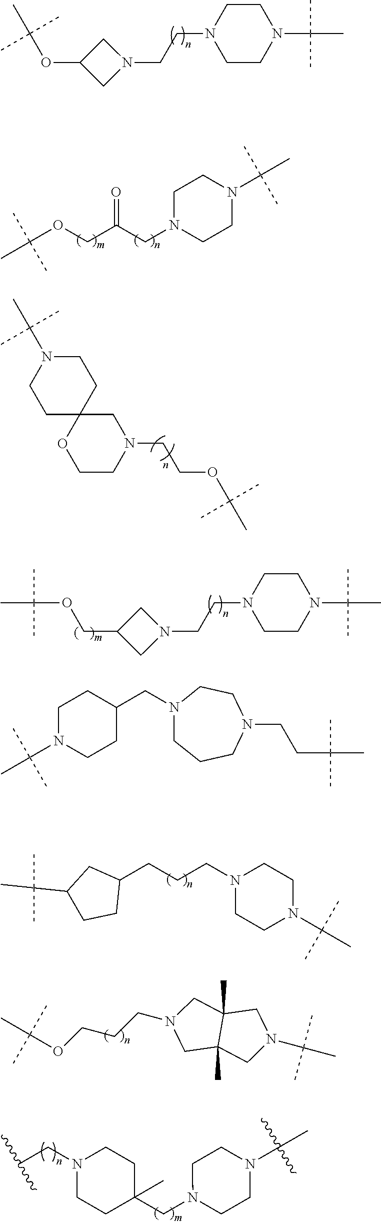

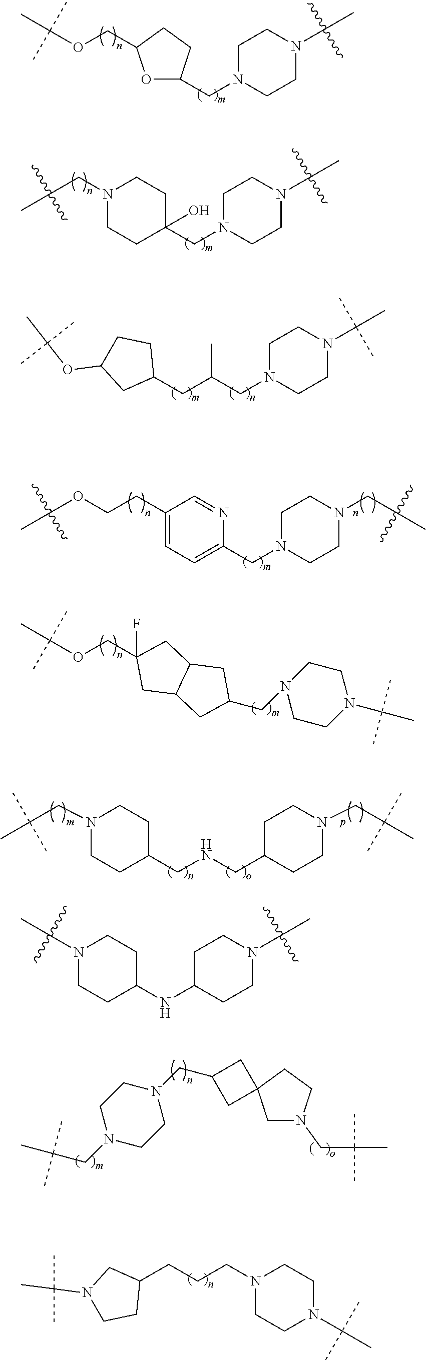

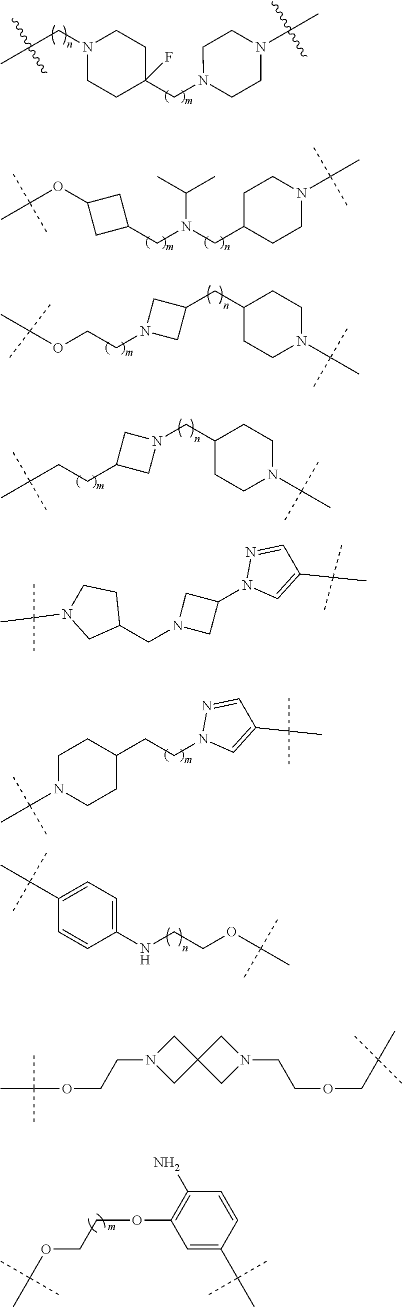

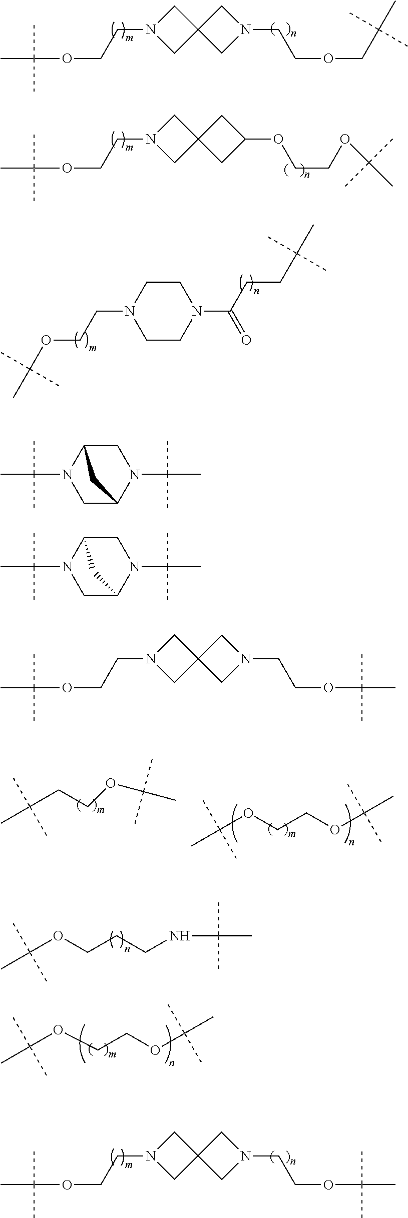

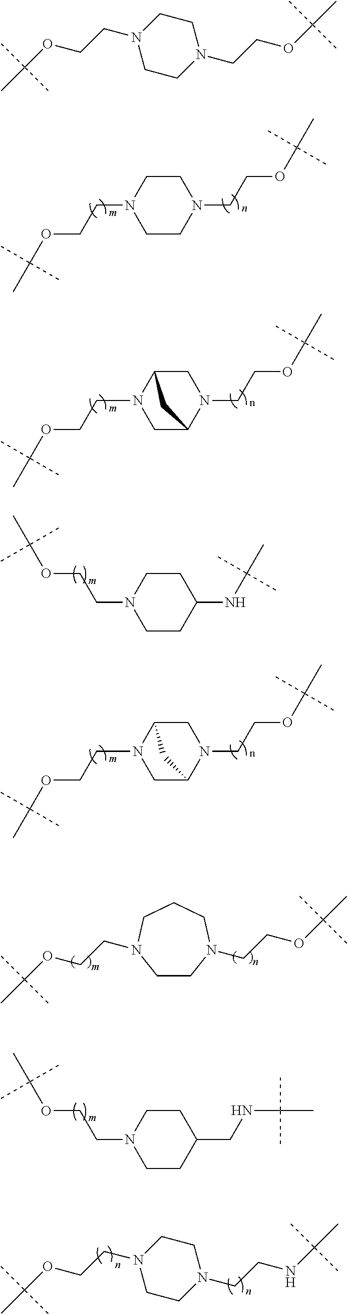

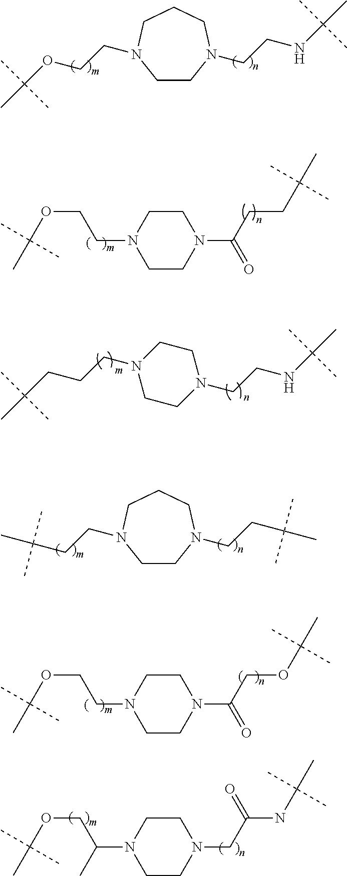

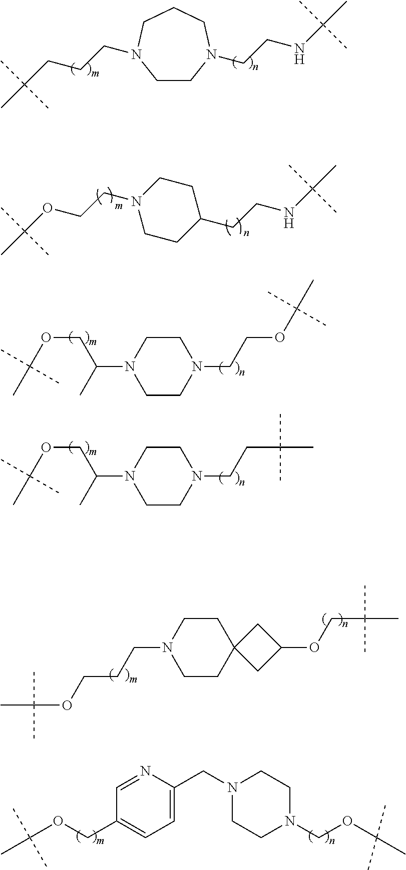

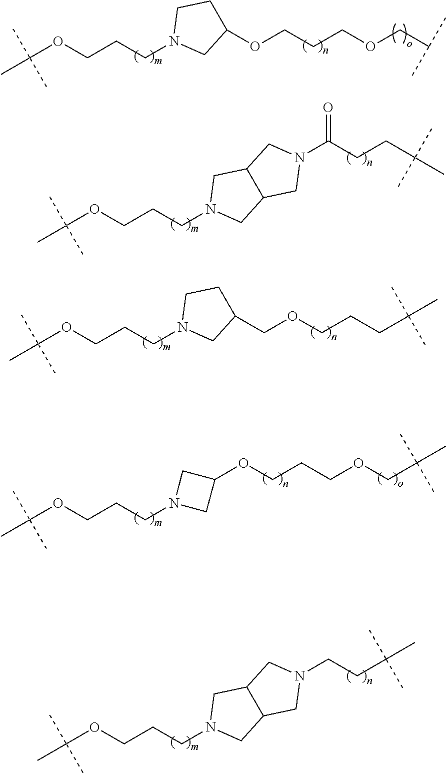

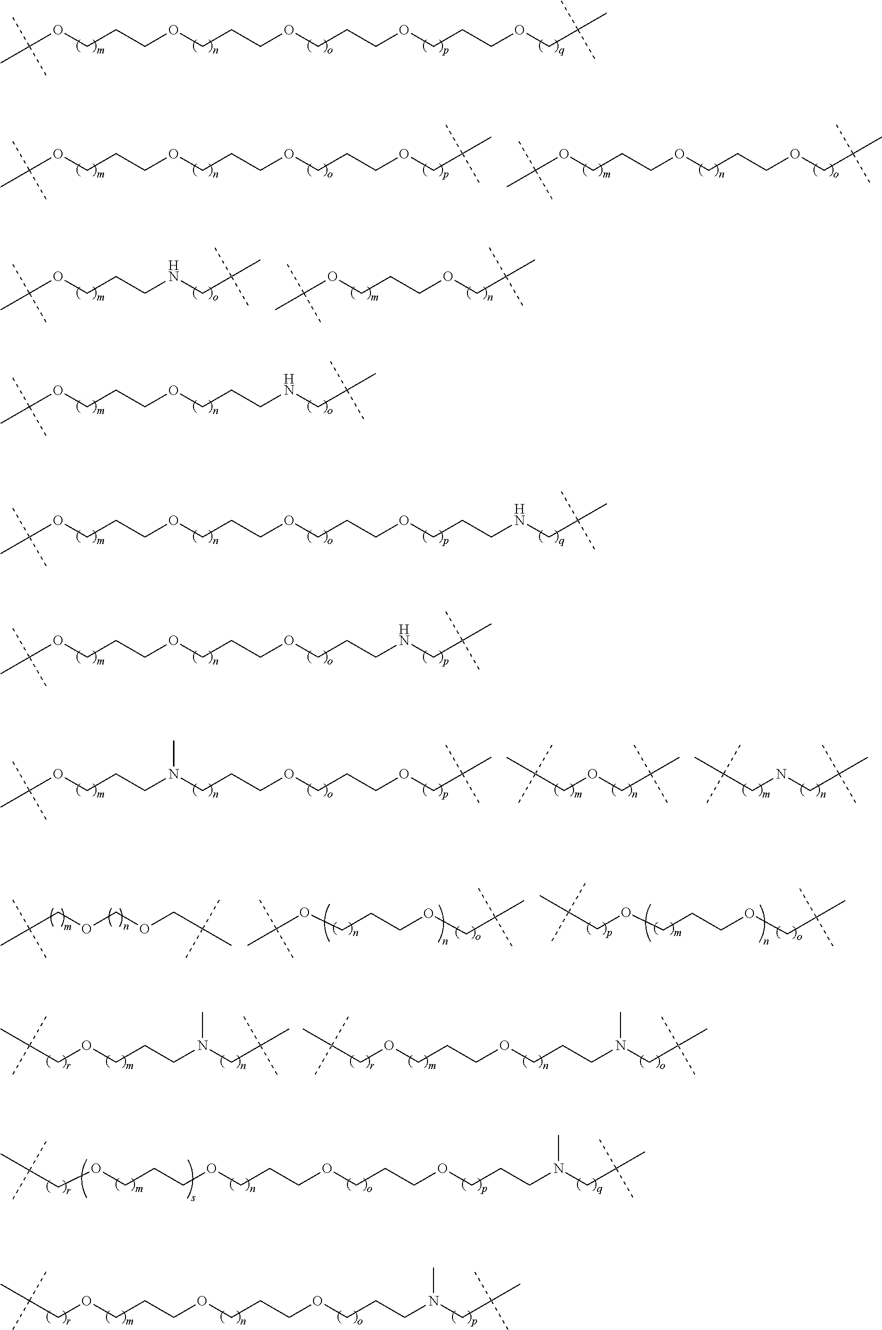

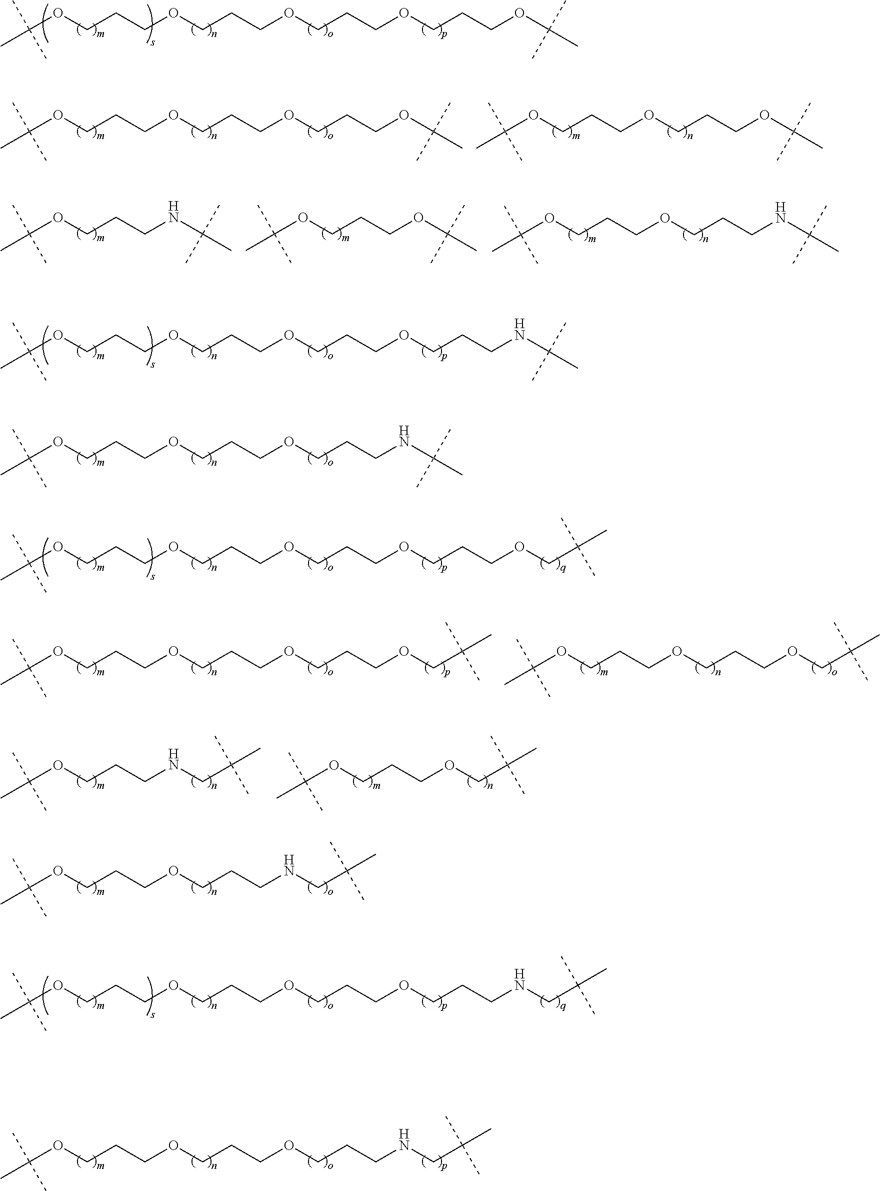

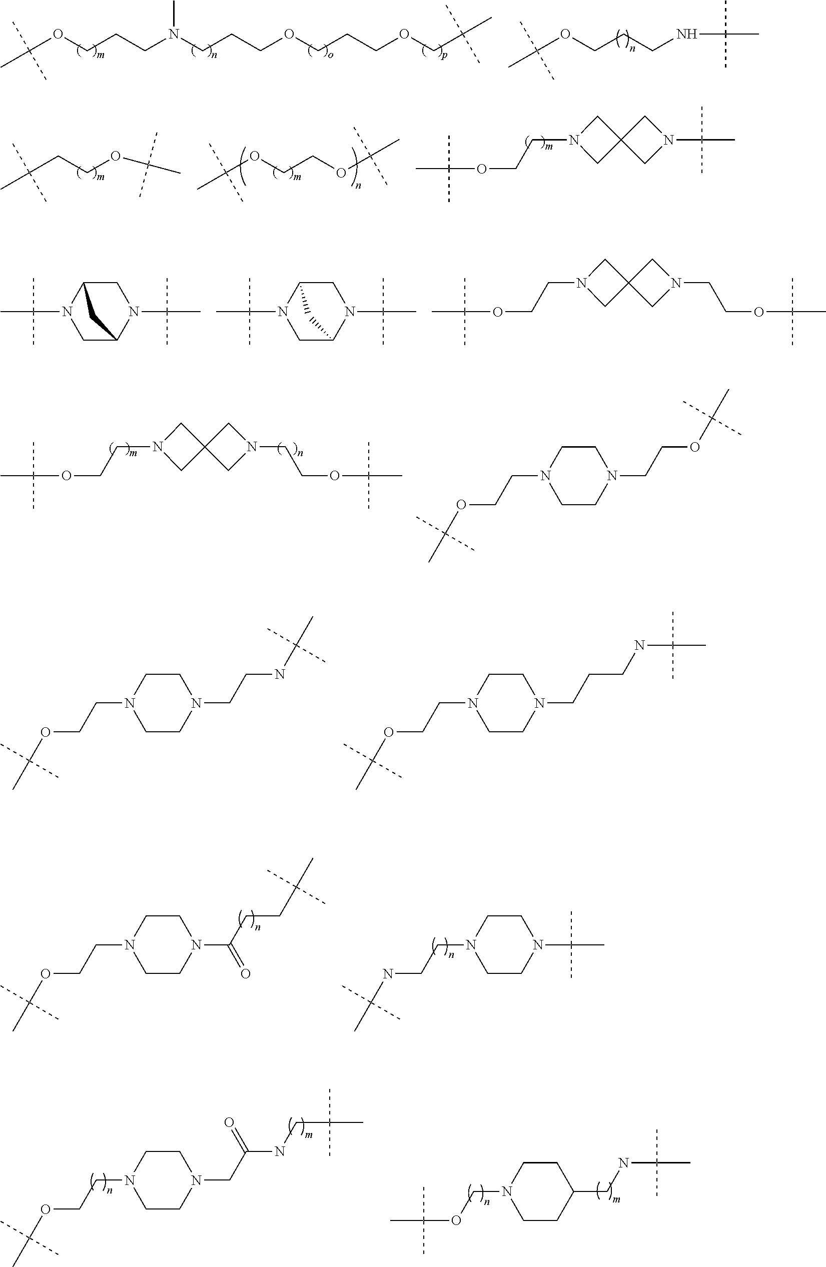

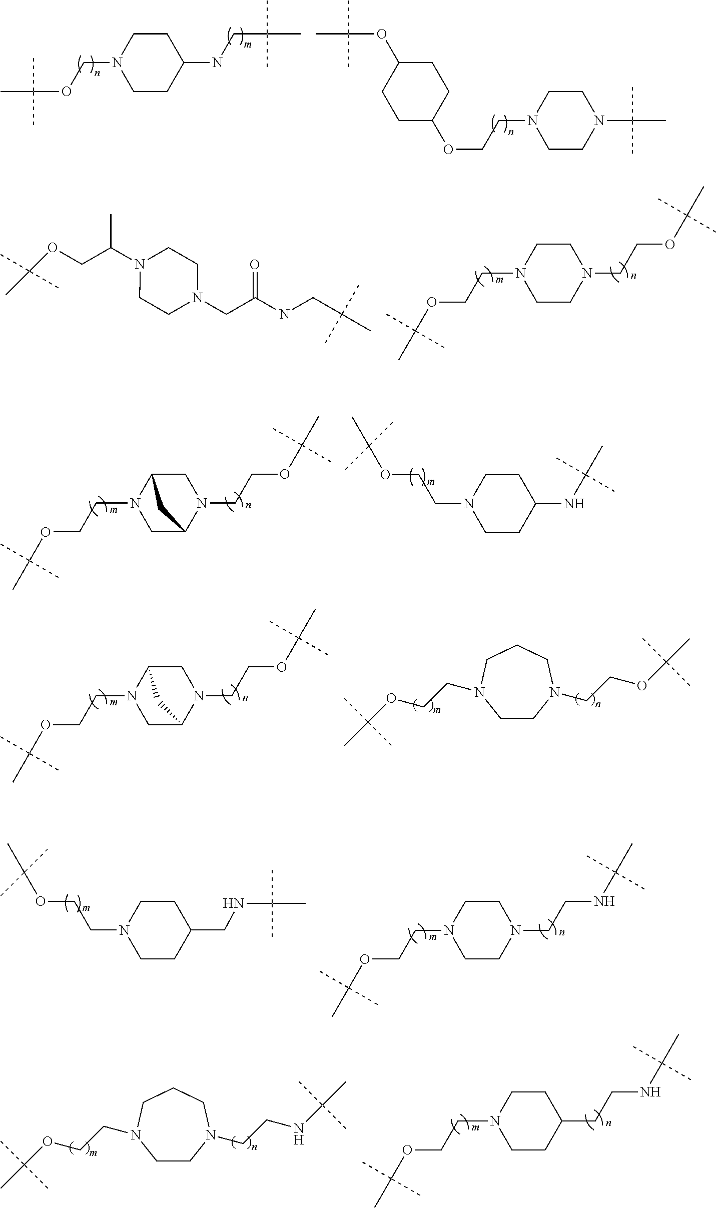

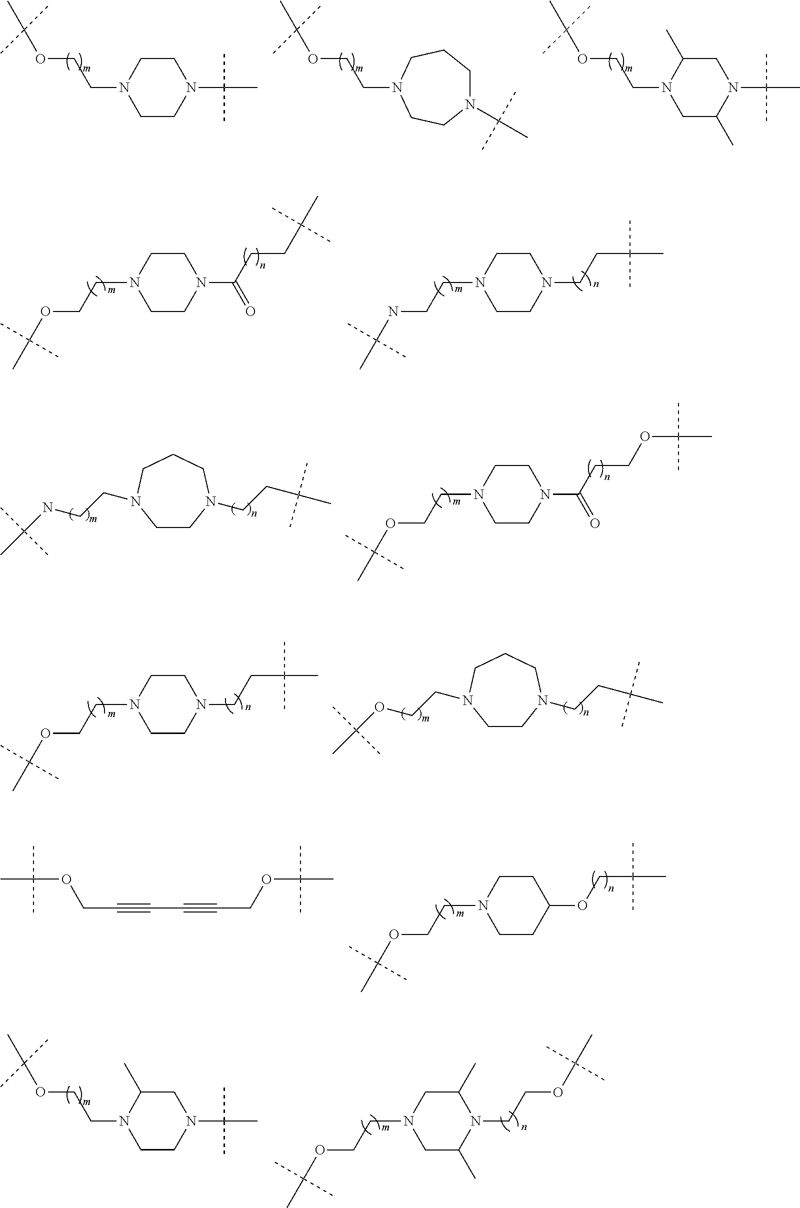

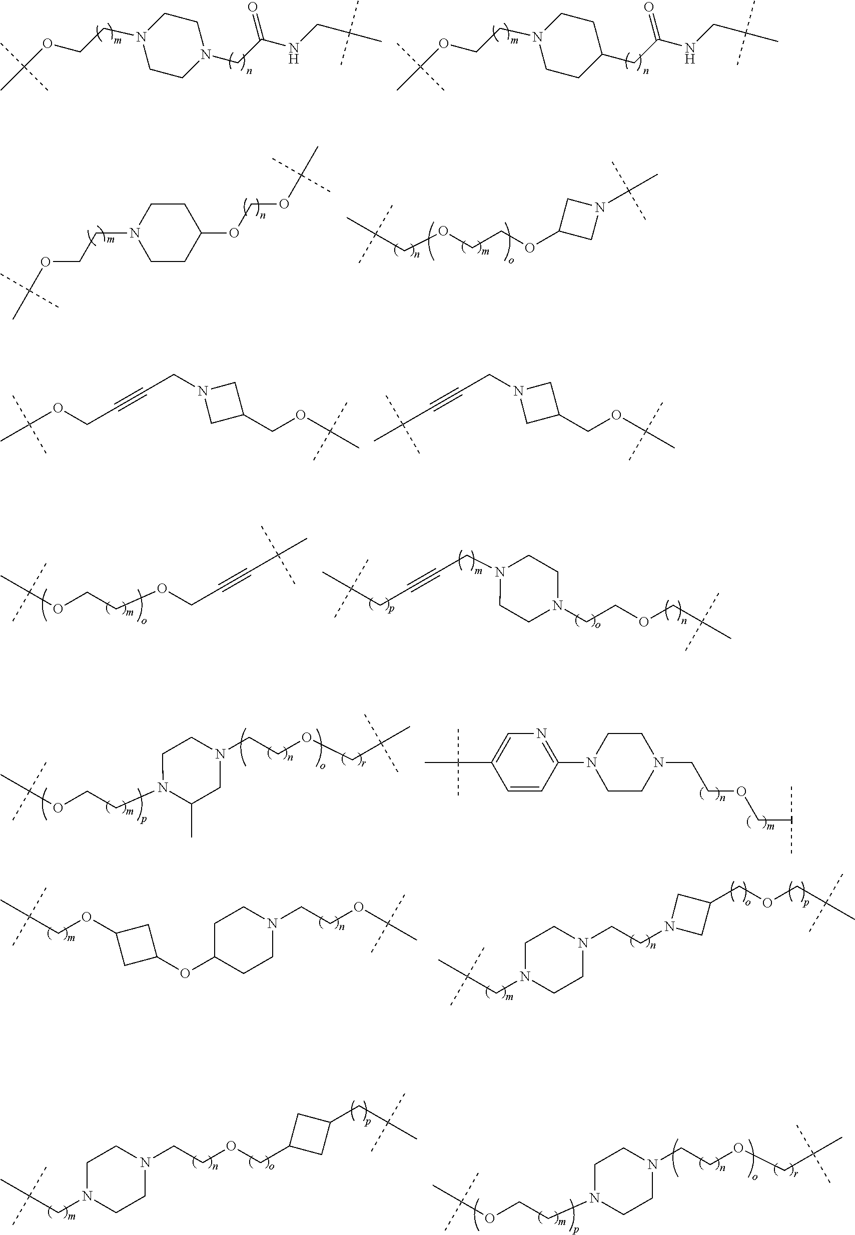

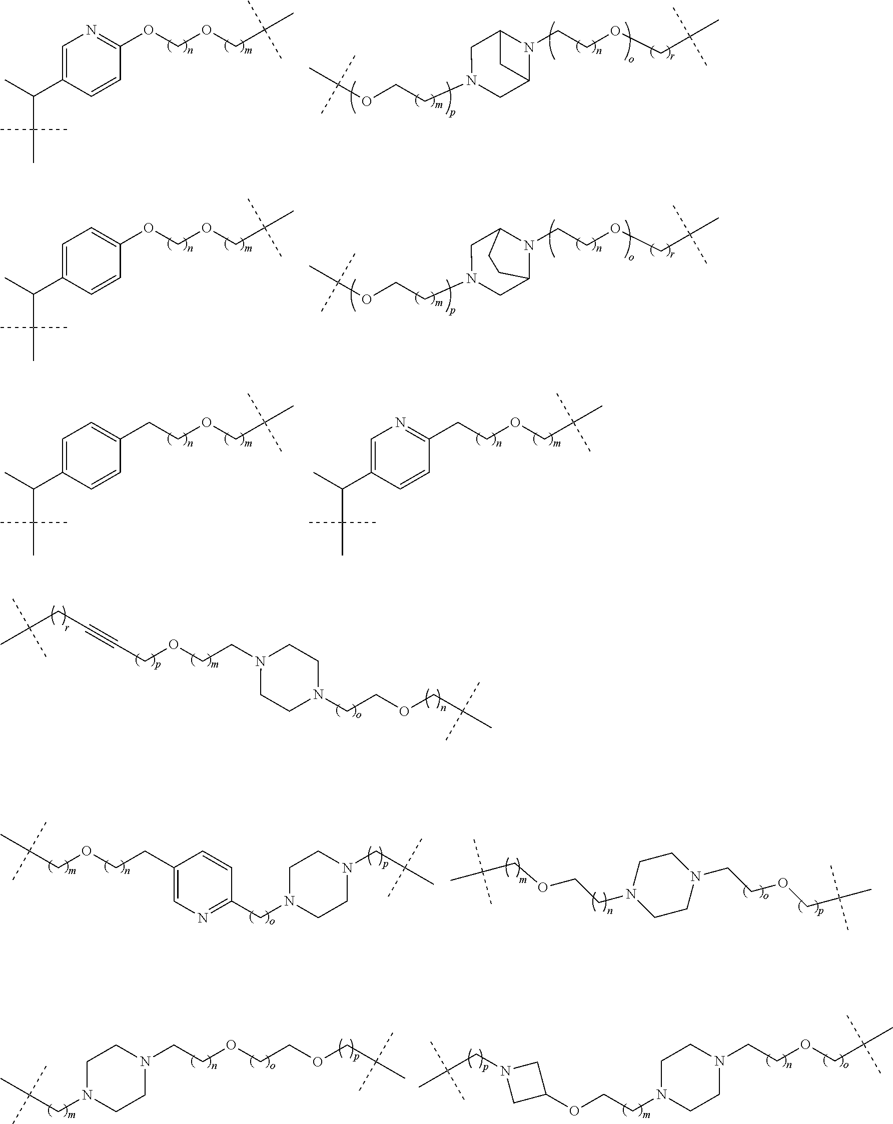

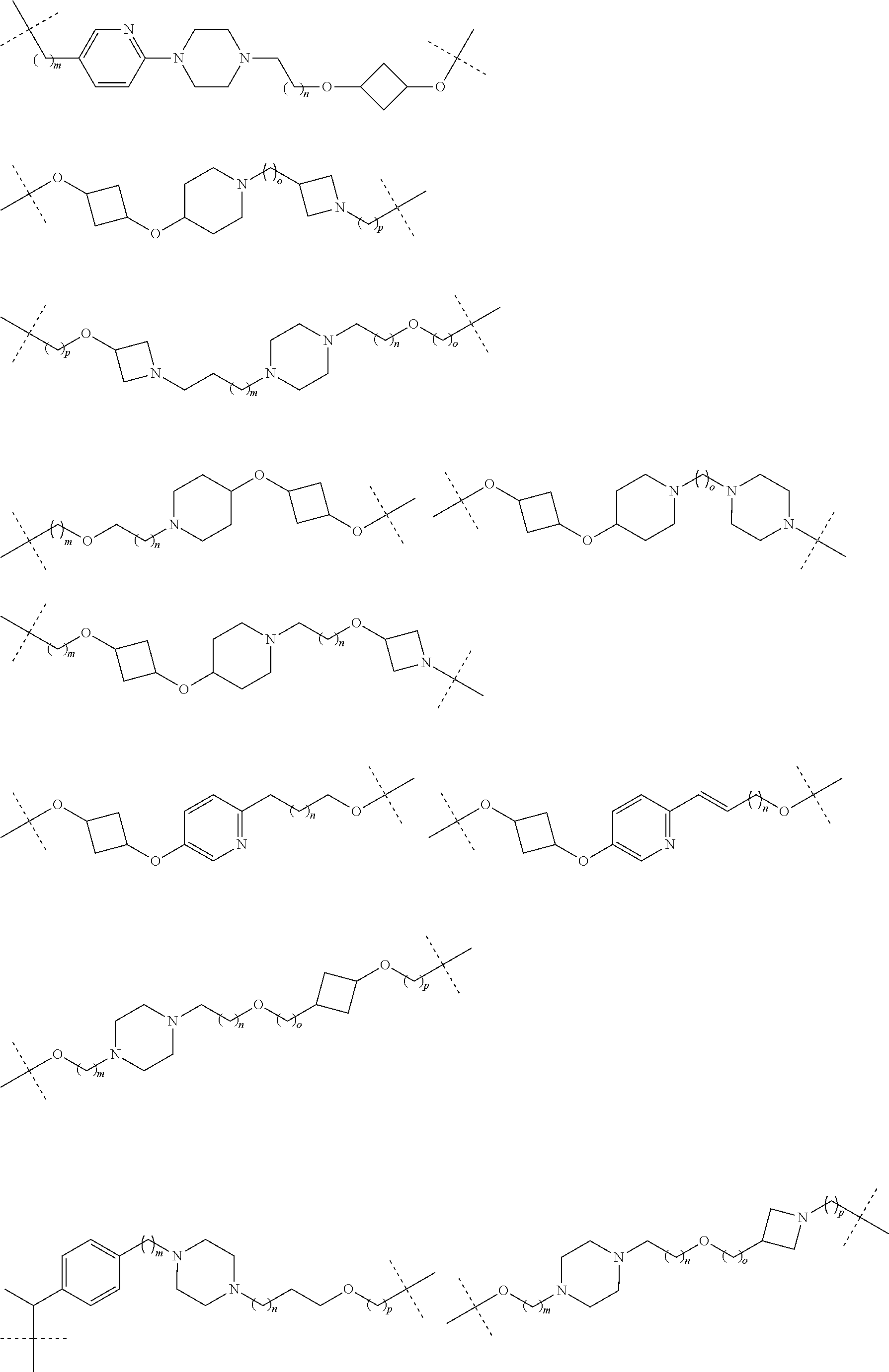

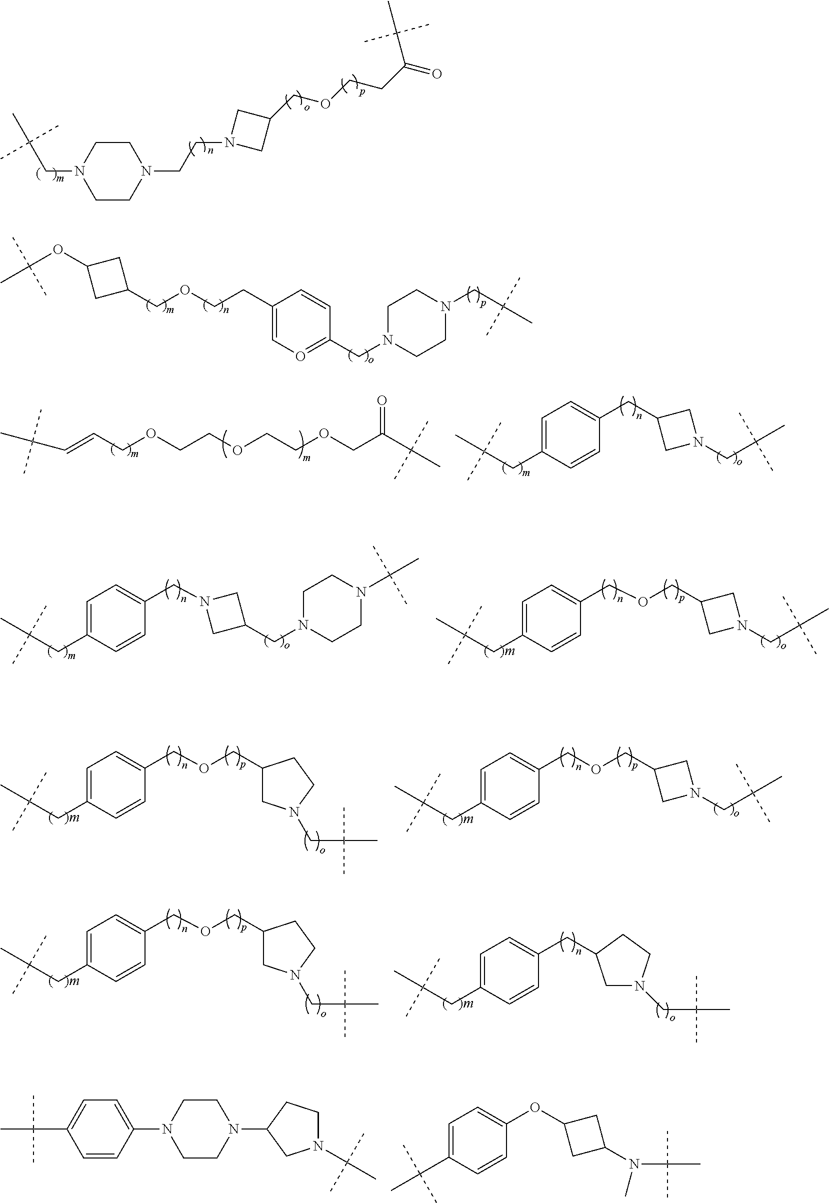

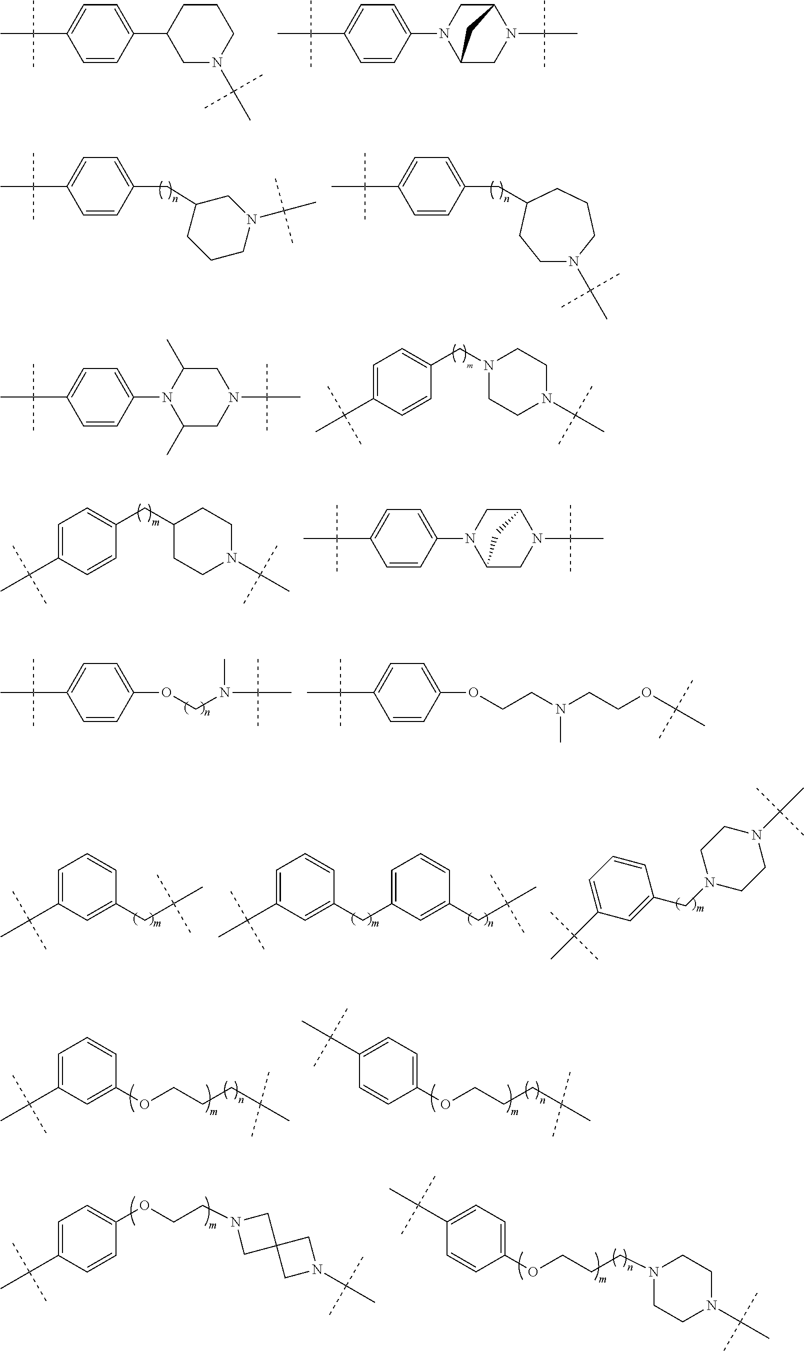

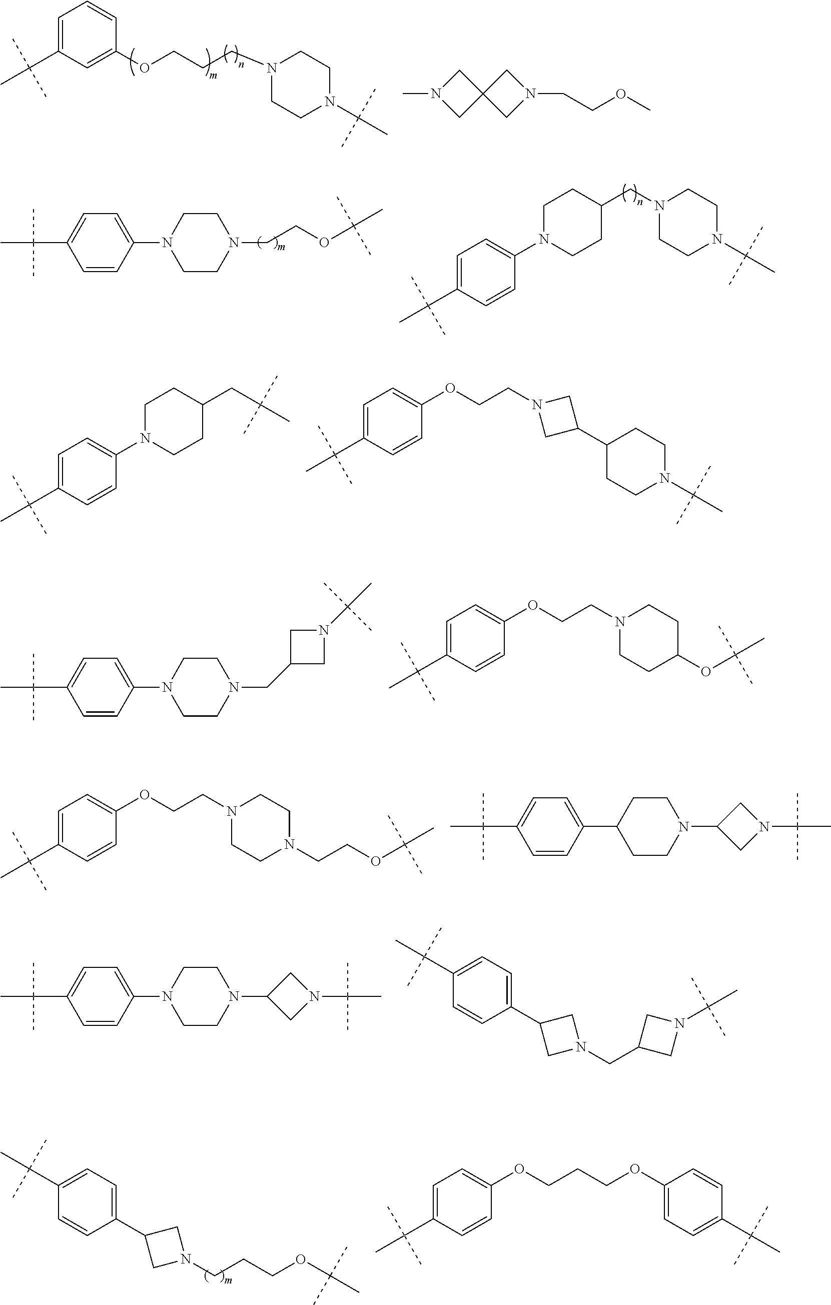

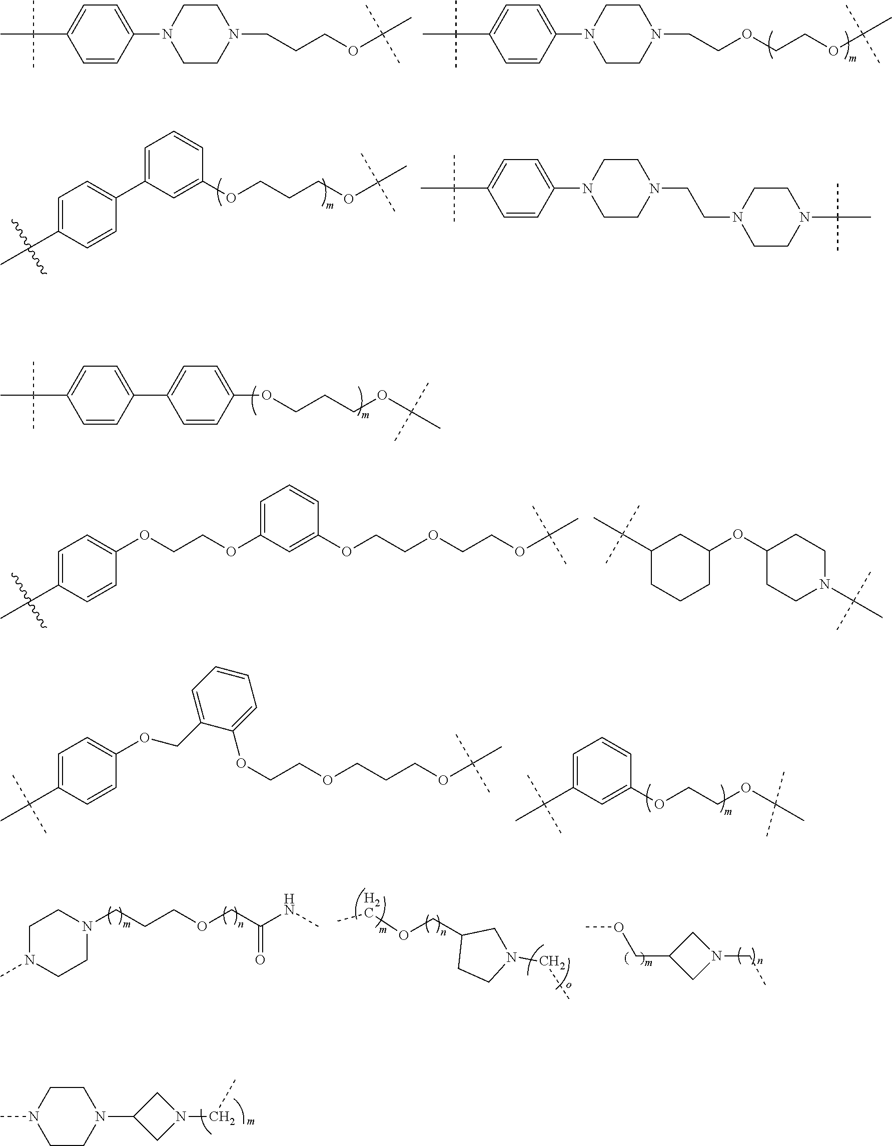

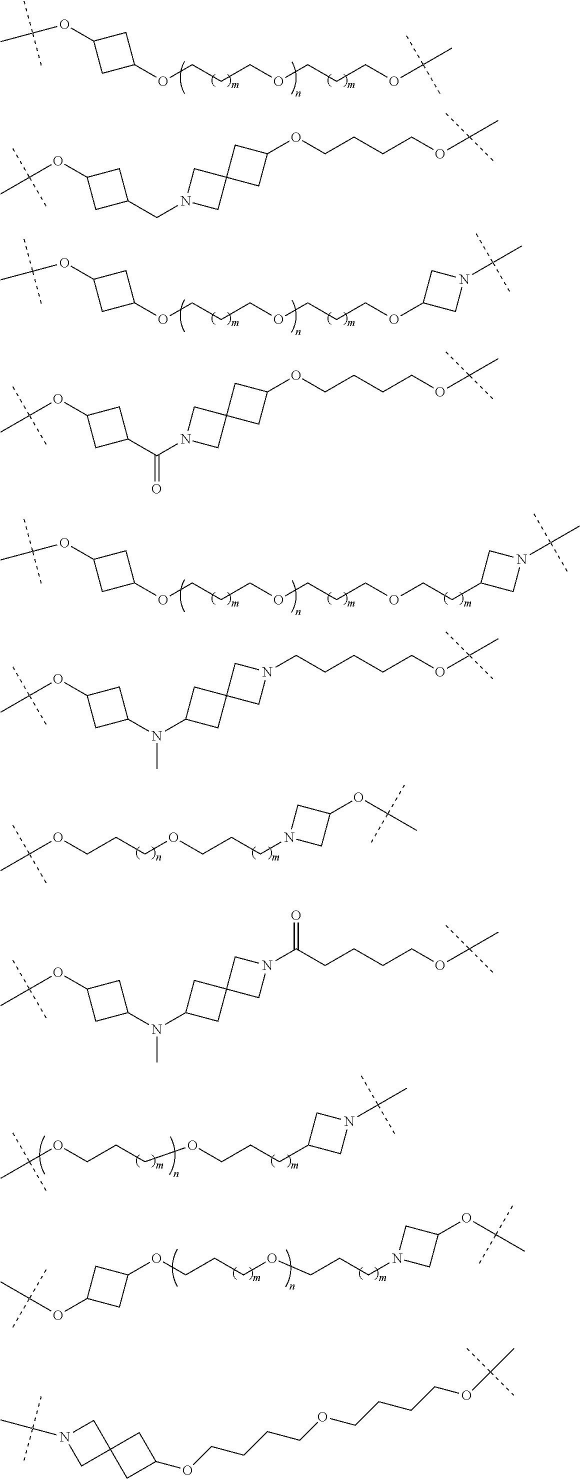

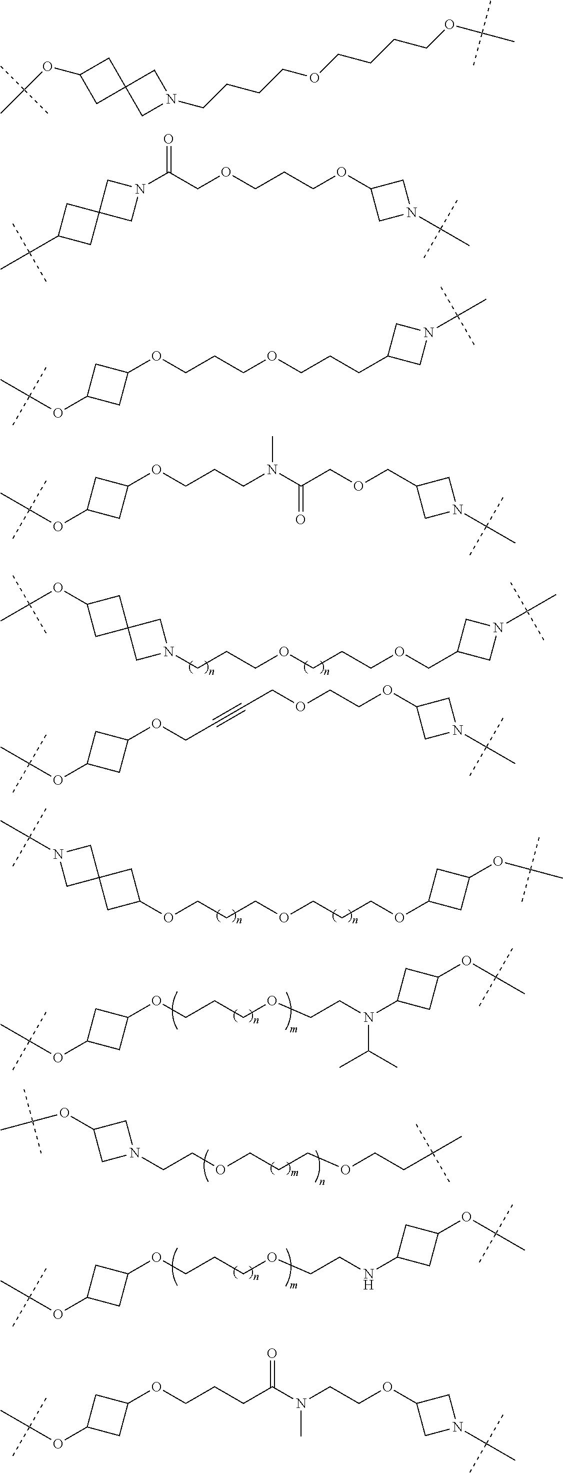

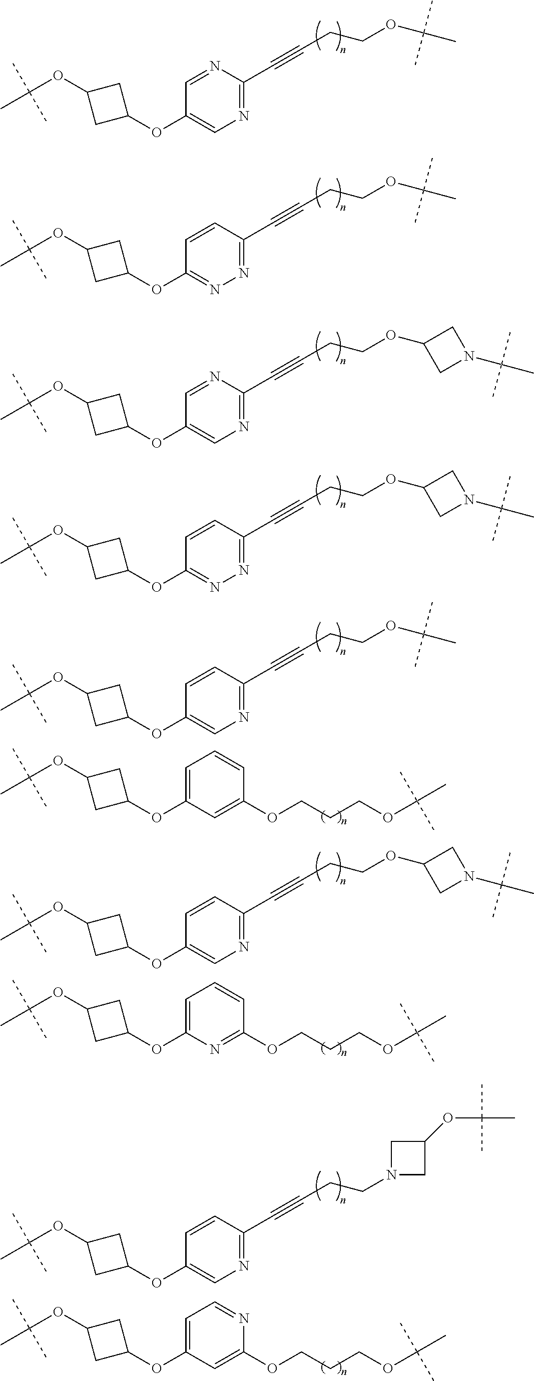

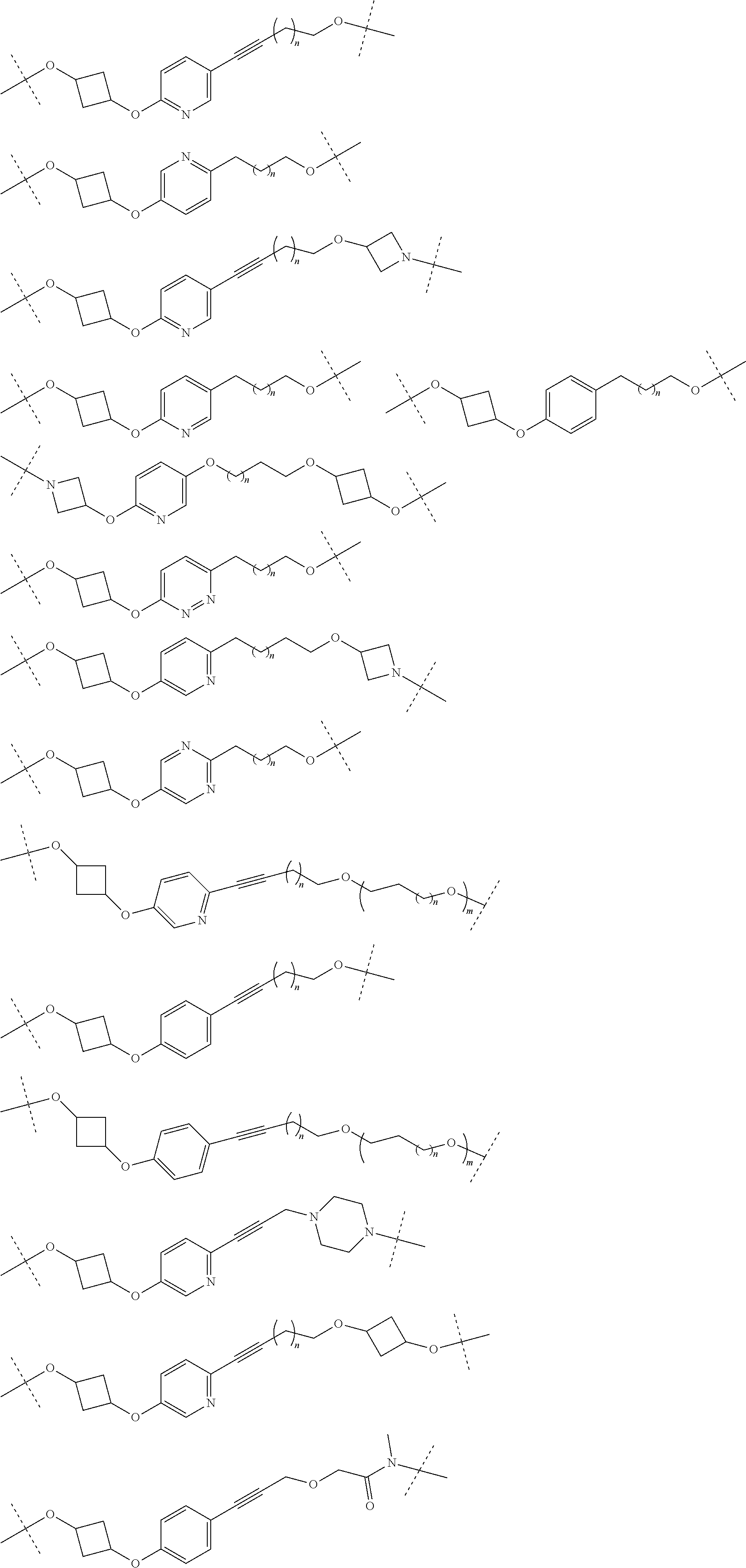

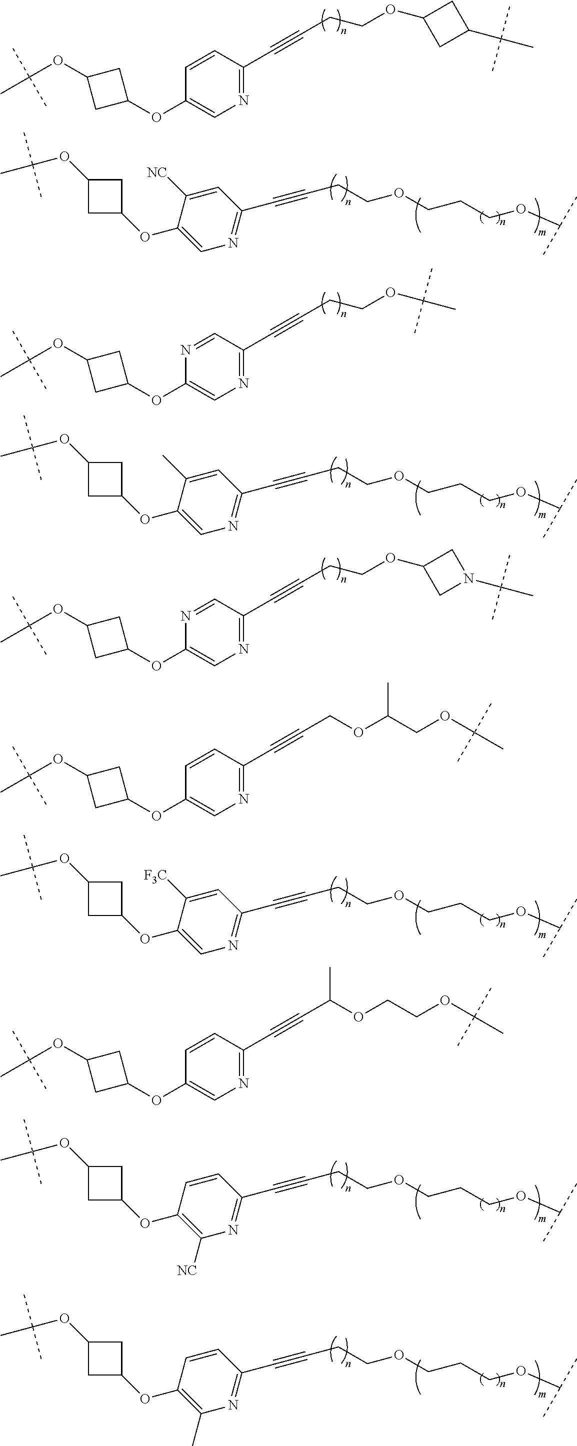

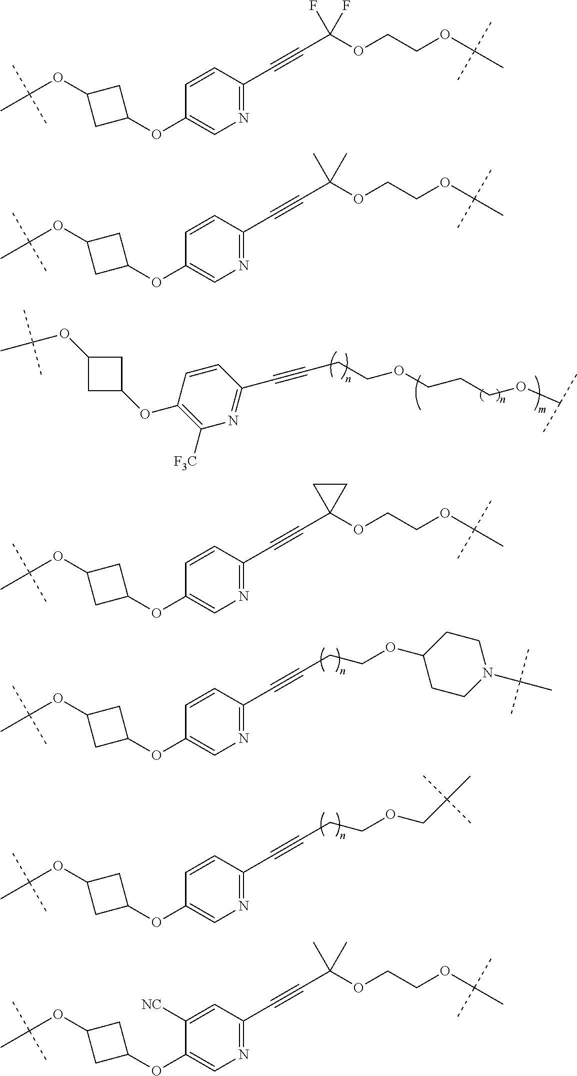

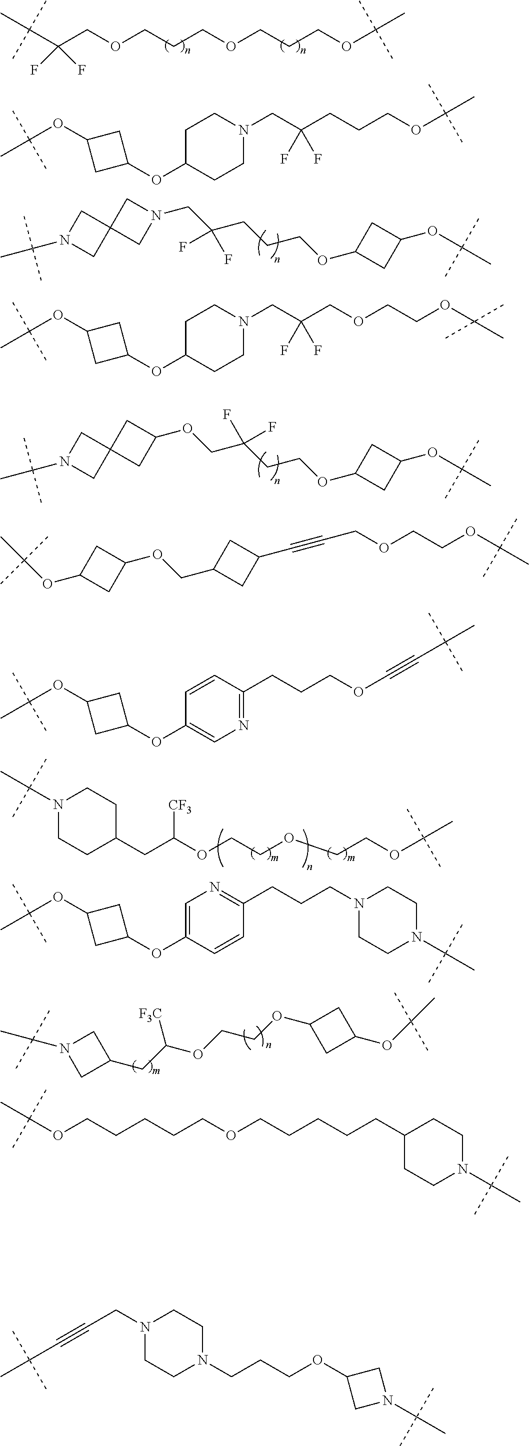

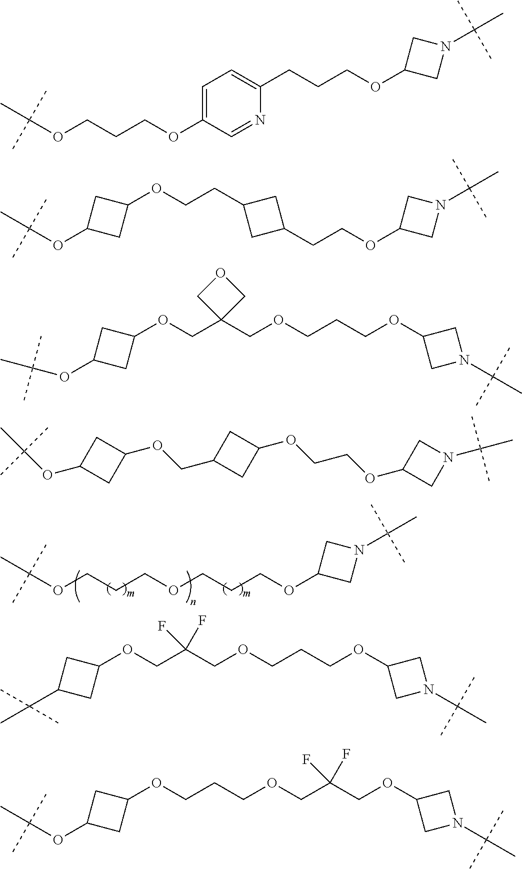

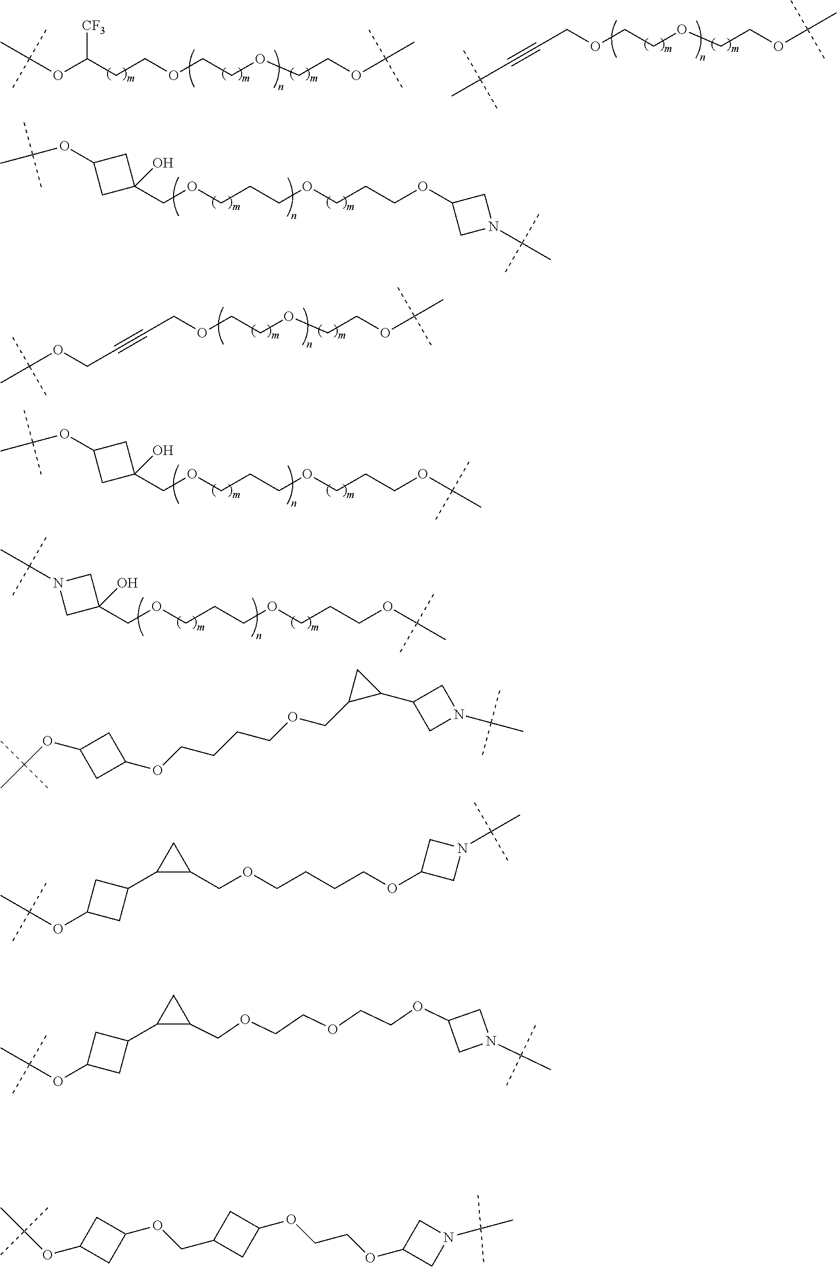

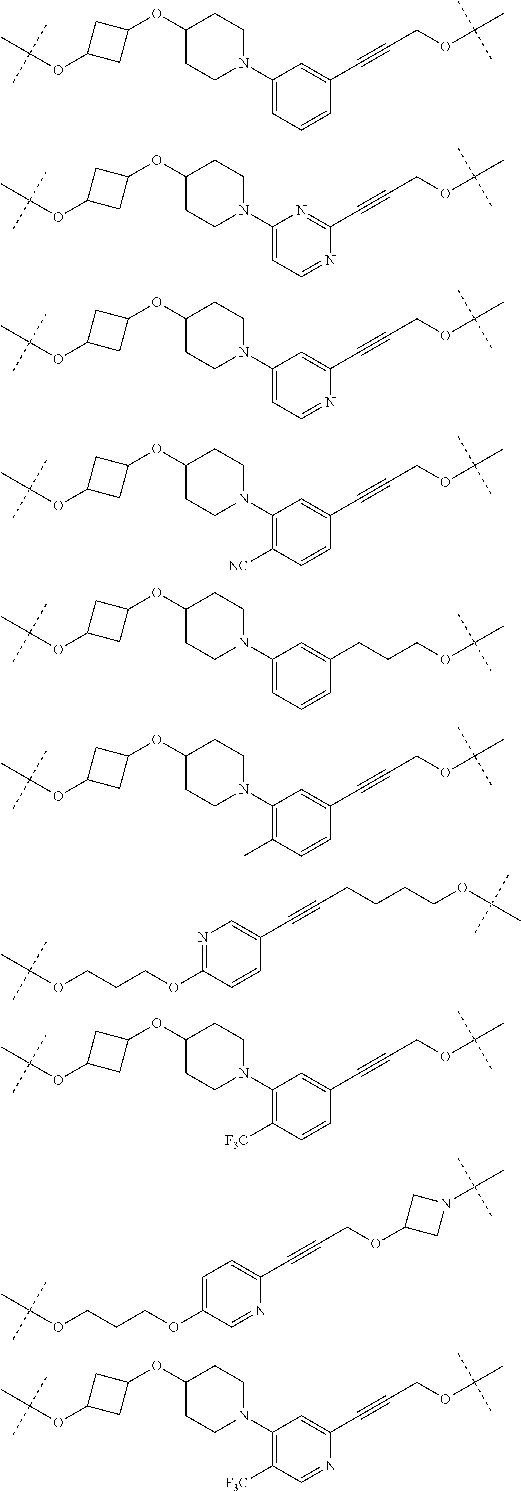

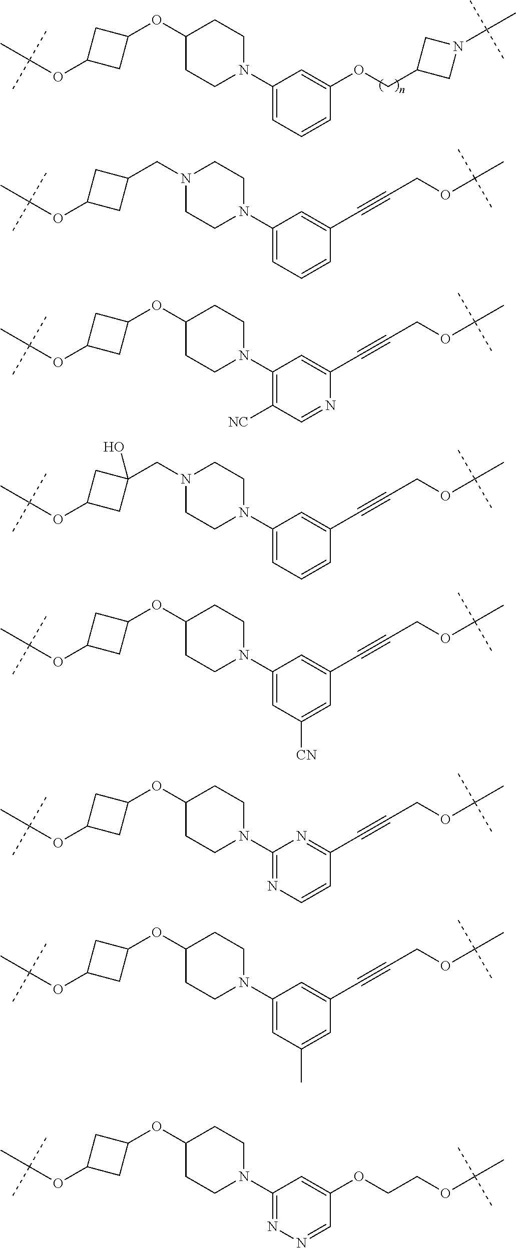

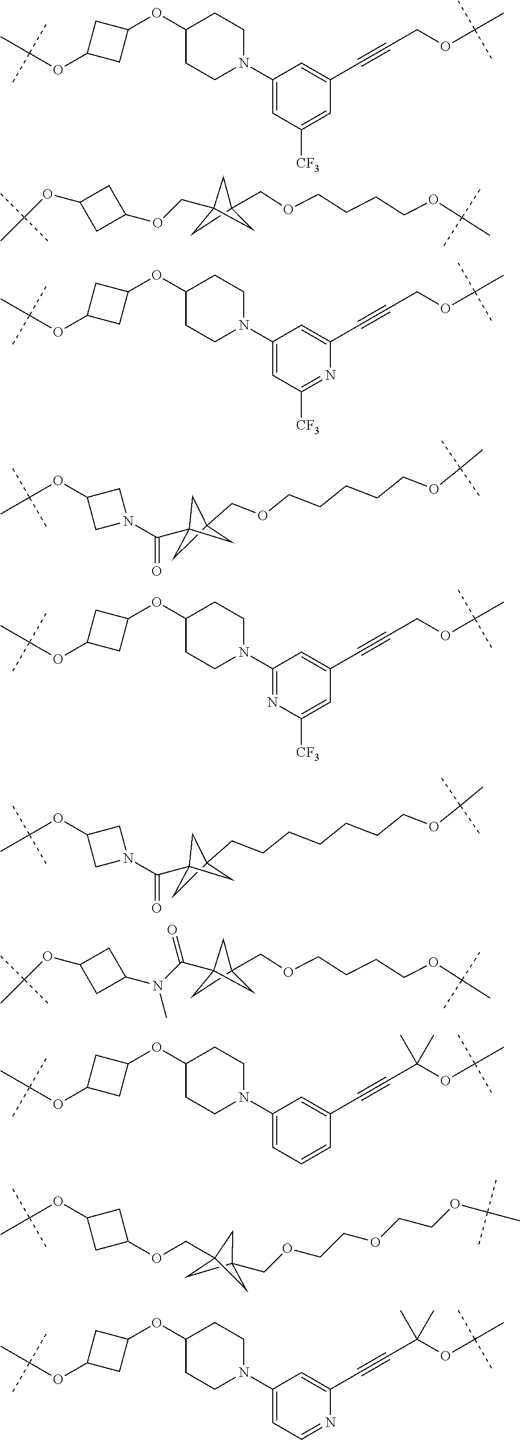

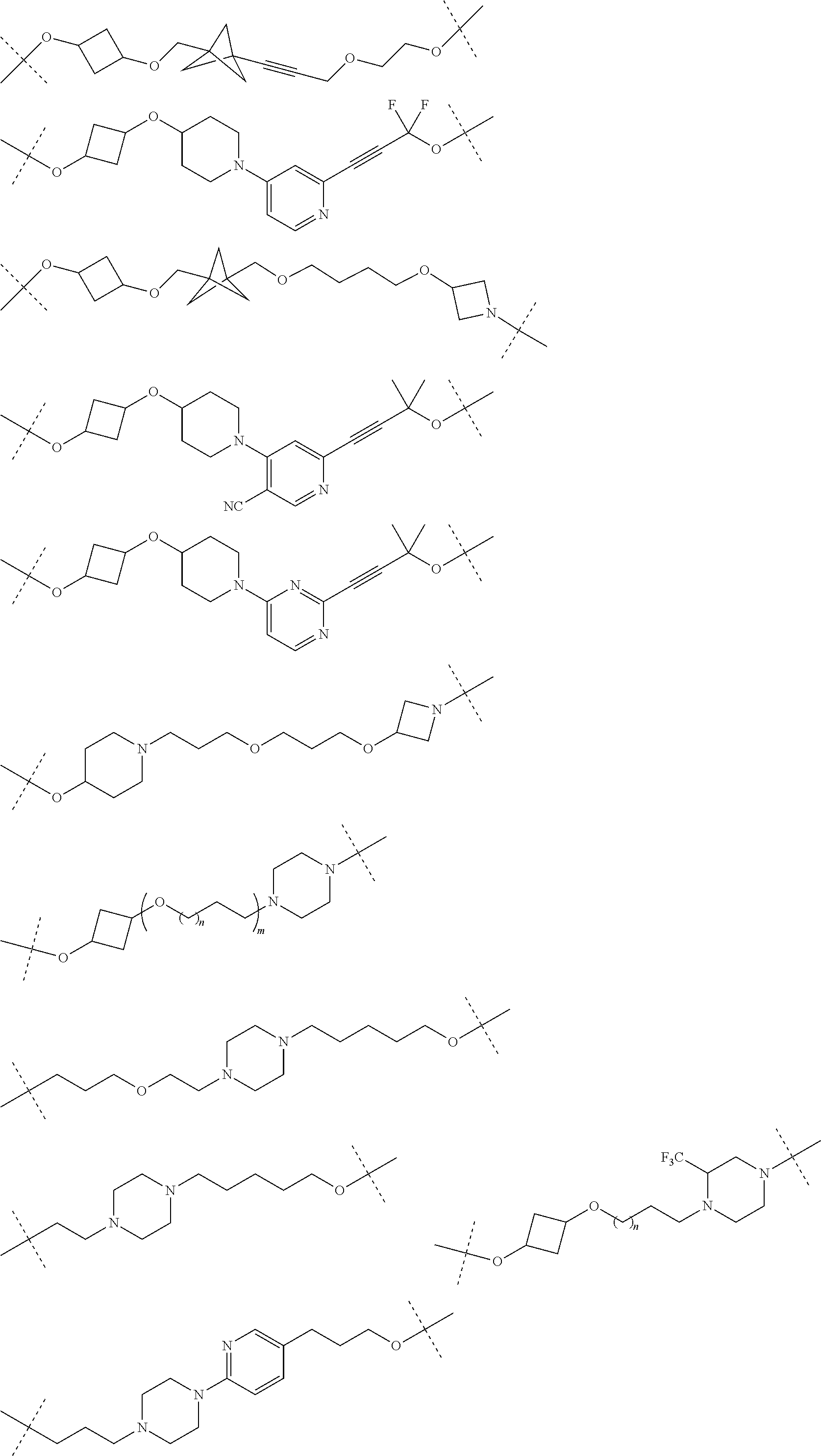

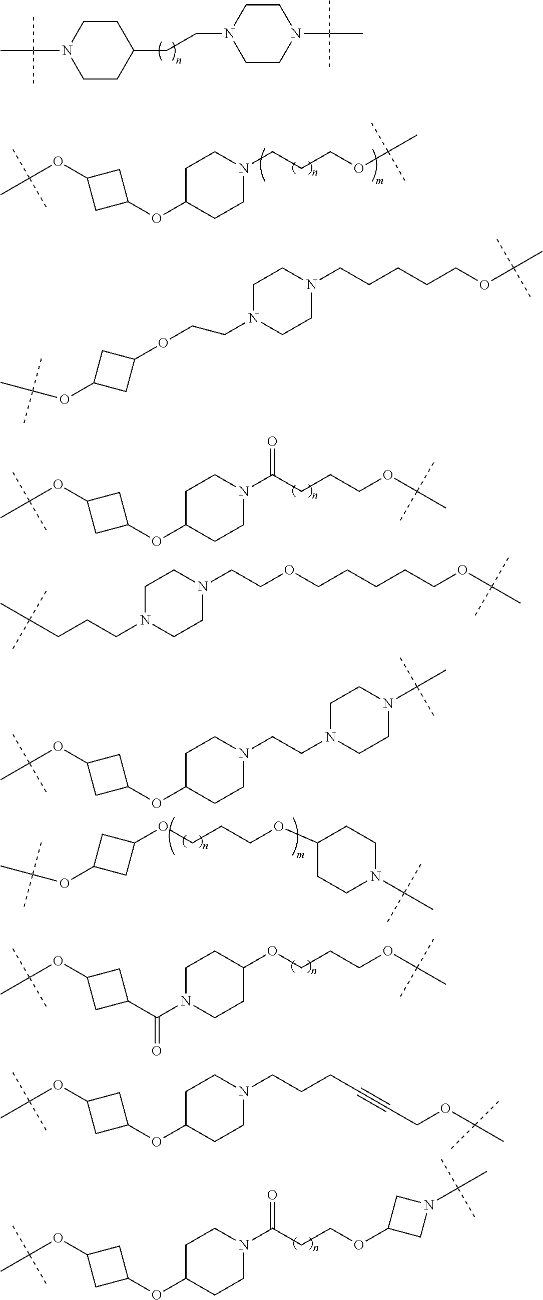

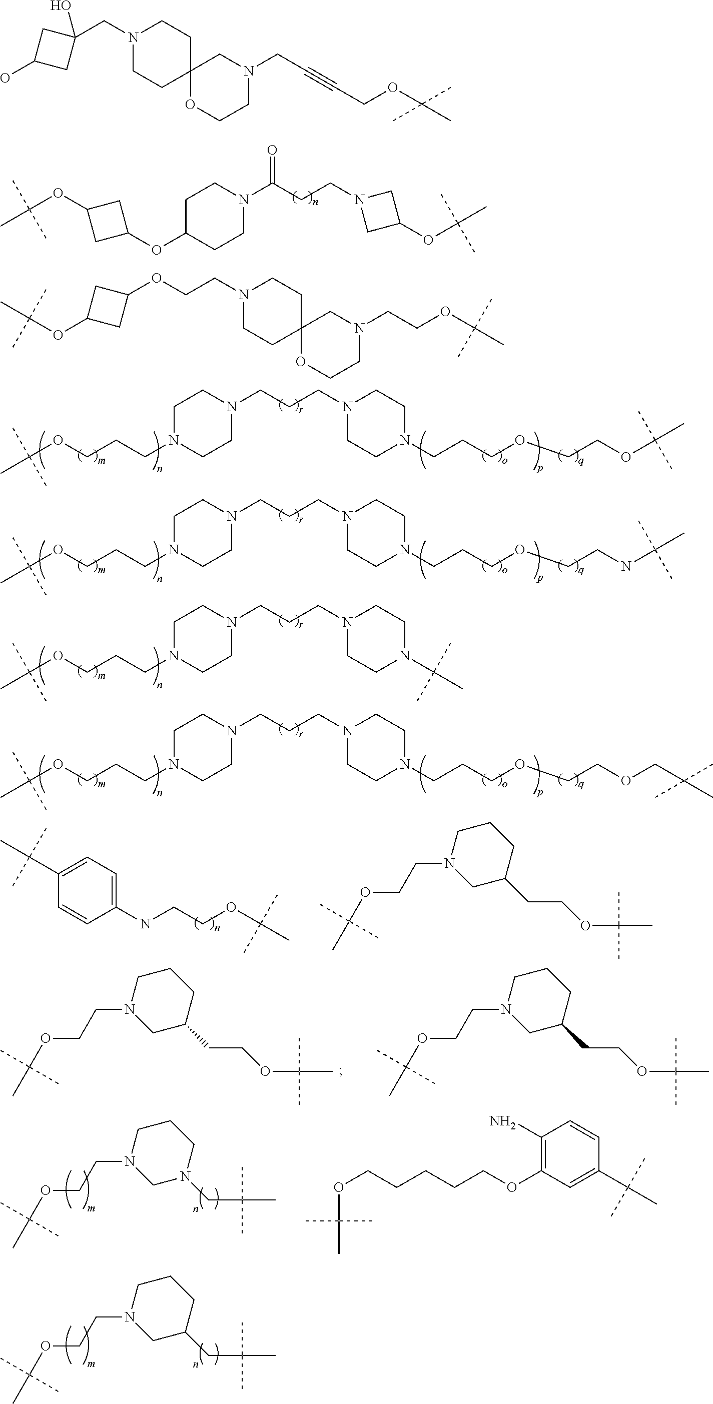

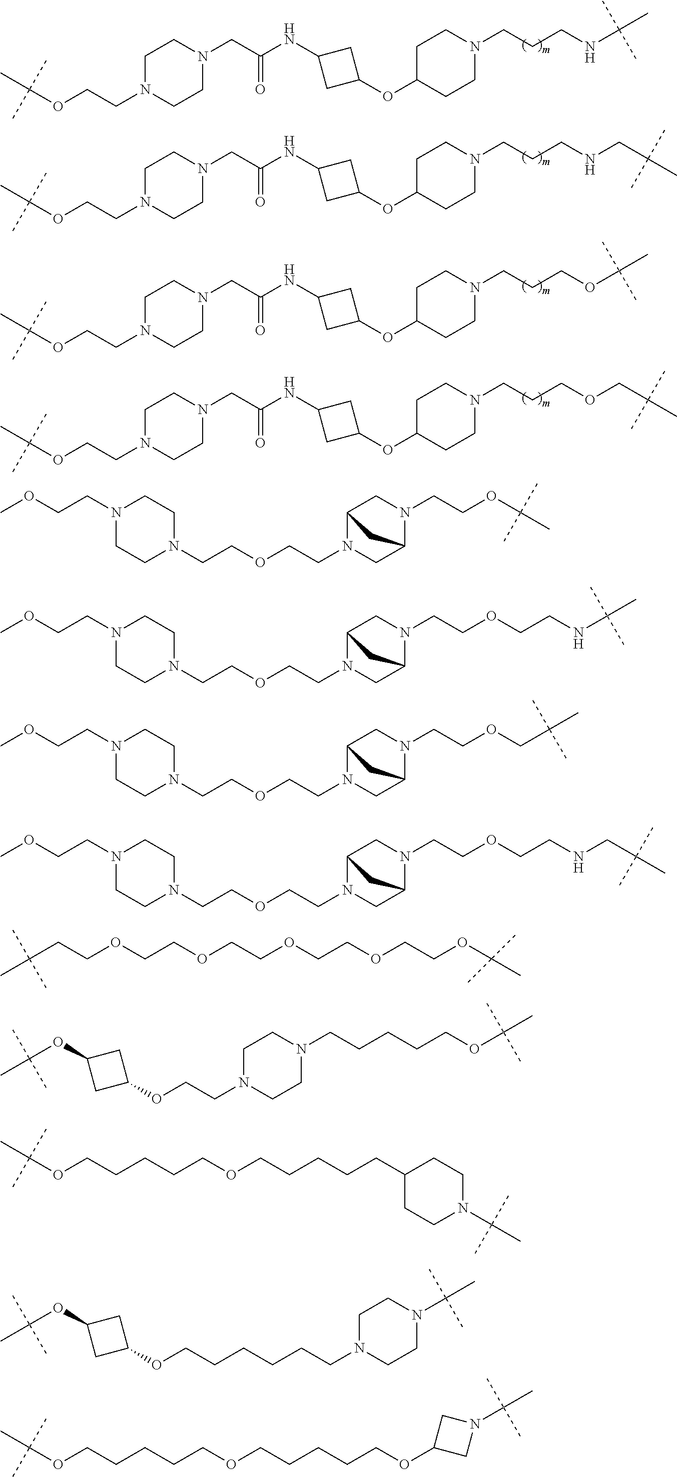

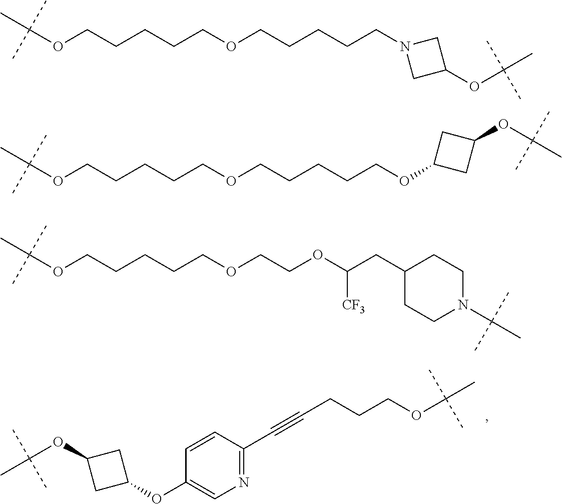

wherein each m, n, o, p, q, r, and s is independently 0, 1, 2, 3, 4, 5, 6, 7, 8, 9, 10, 11, 12, 13, 14, 15, 16, 17, 18, 19, or 20.

In any aspect or embodiment described herein, the unit A^{L} of linker (L) is selected from:

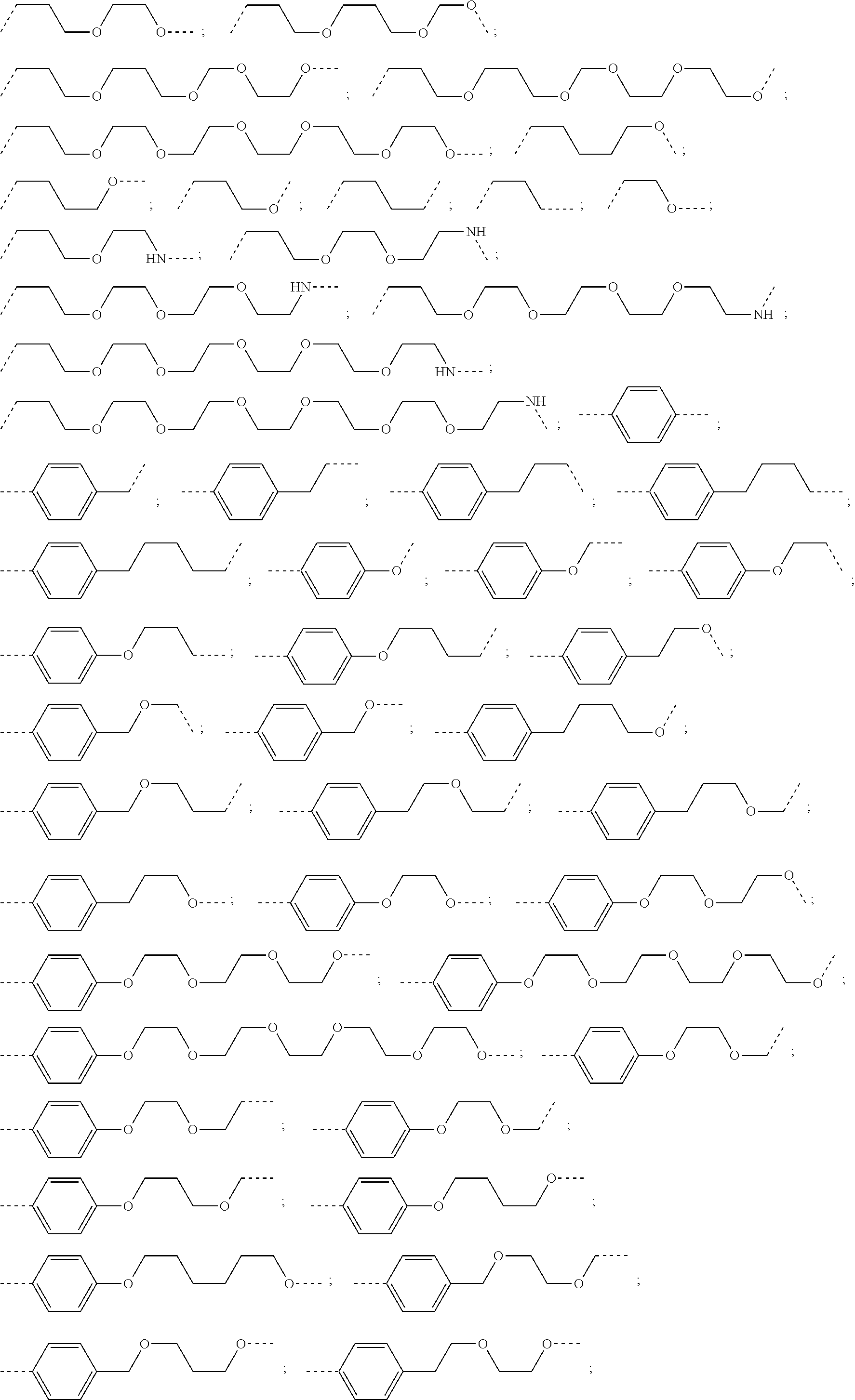

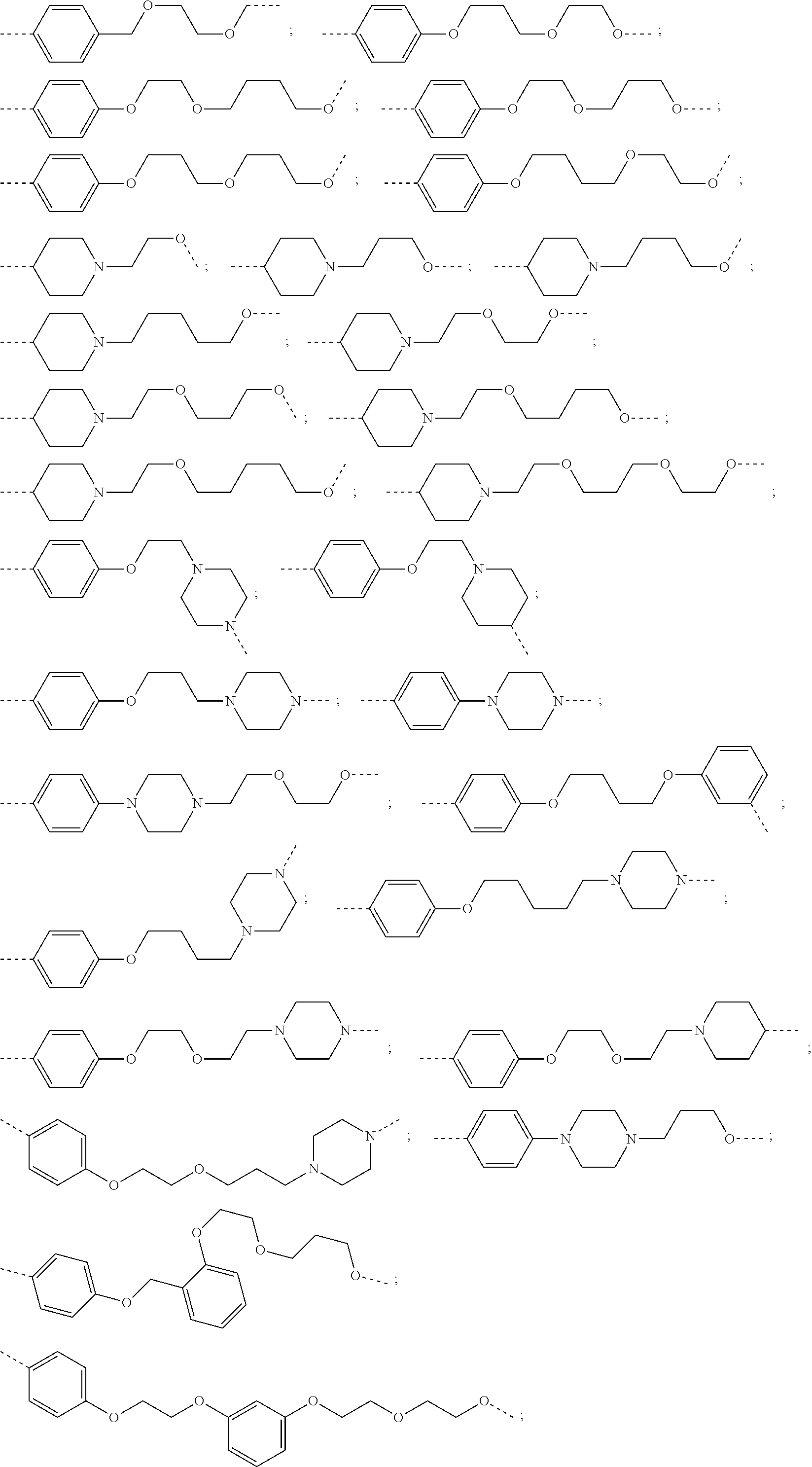

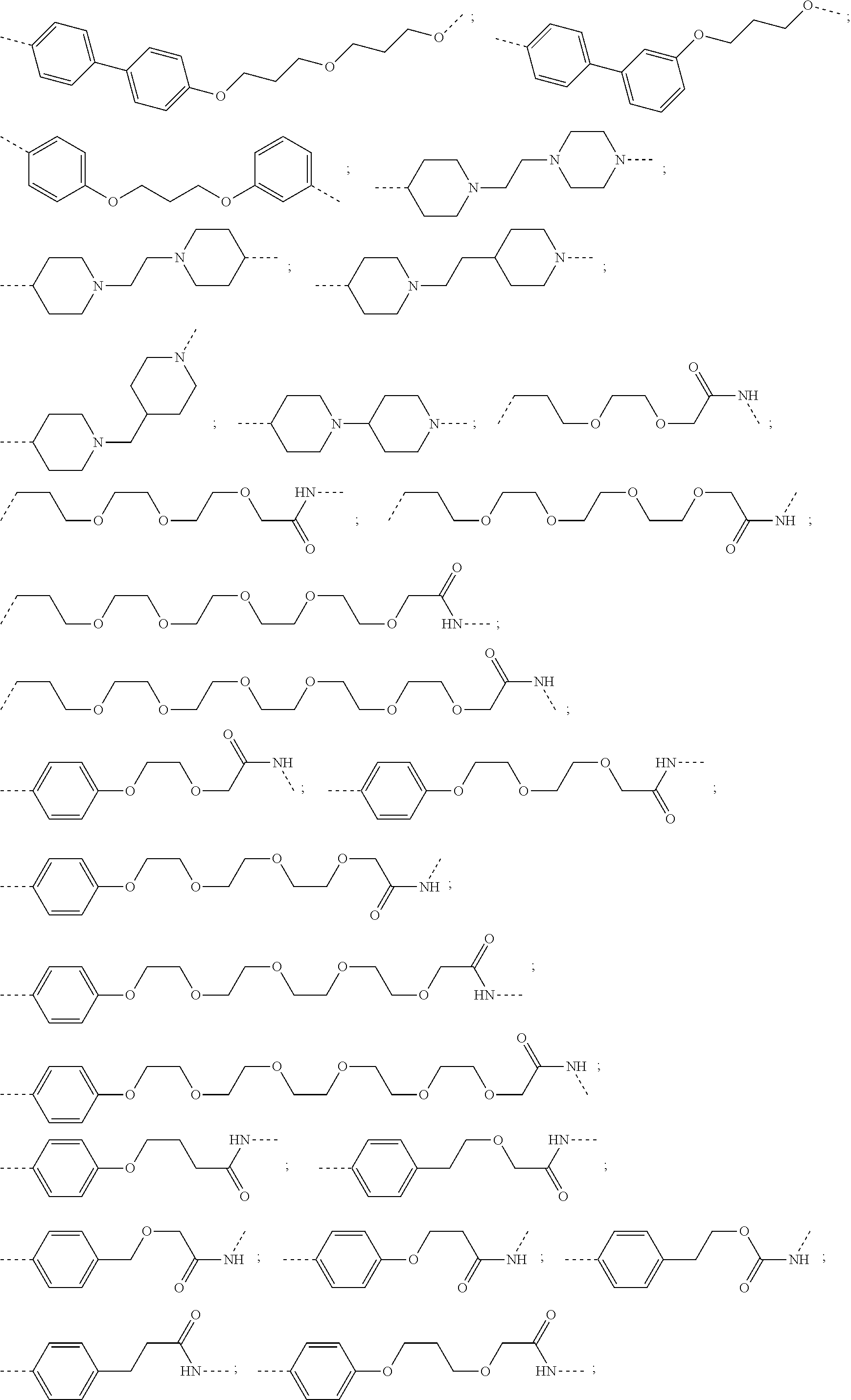

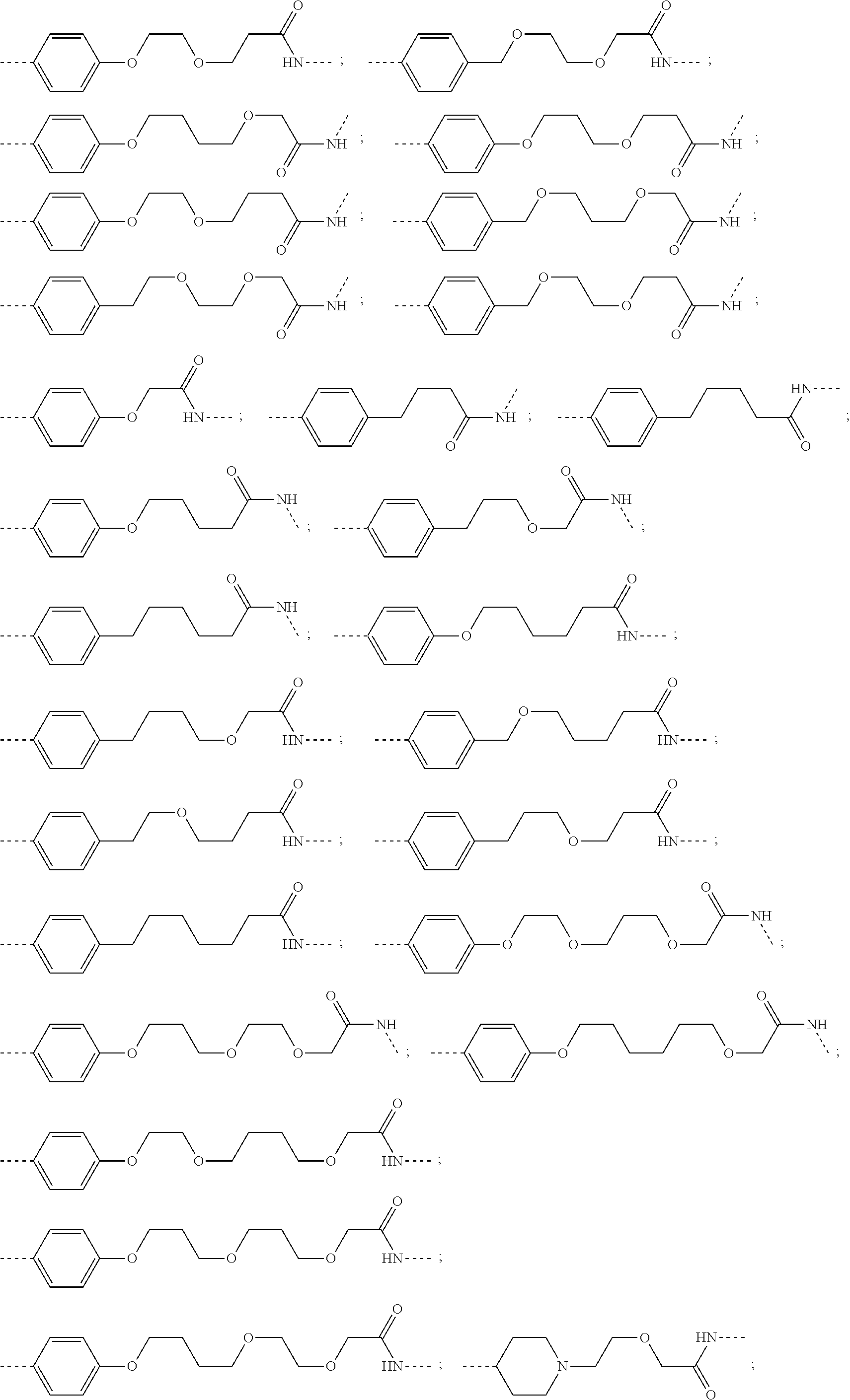

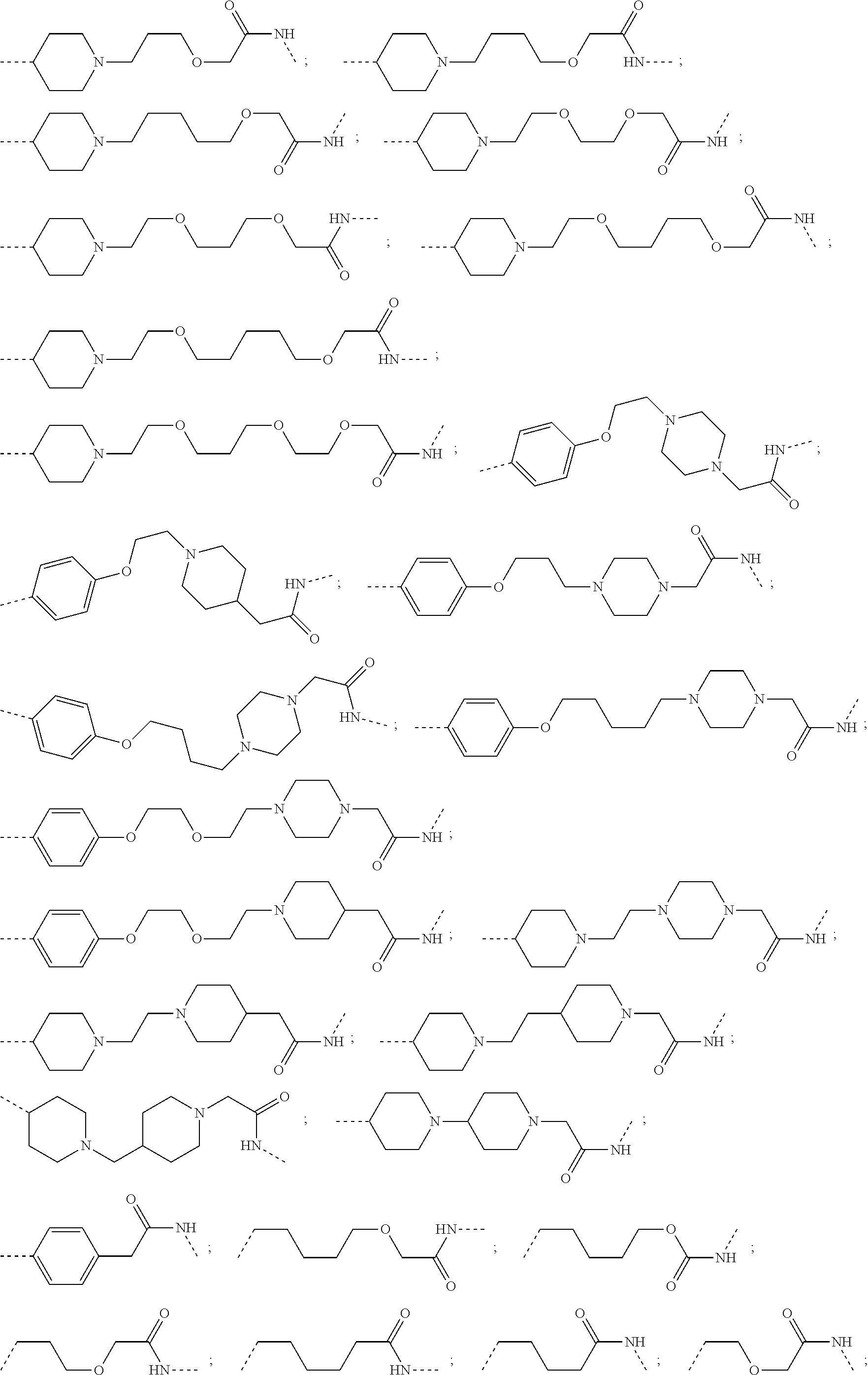

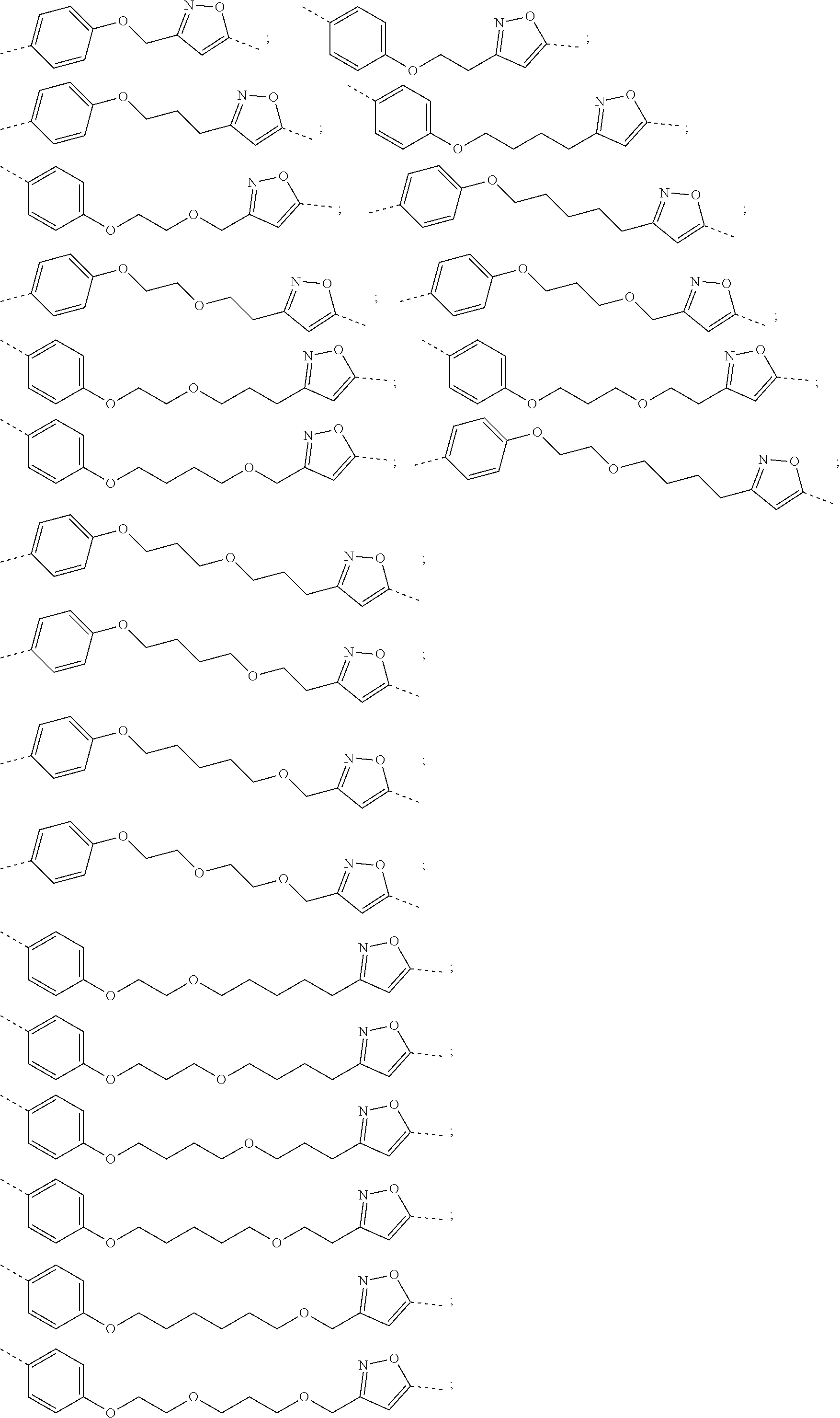

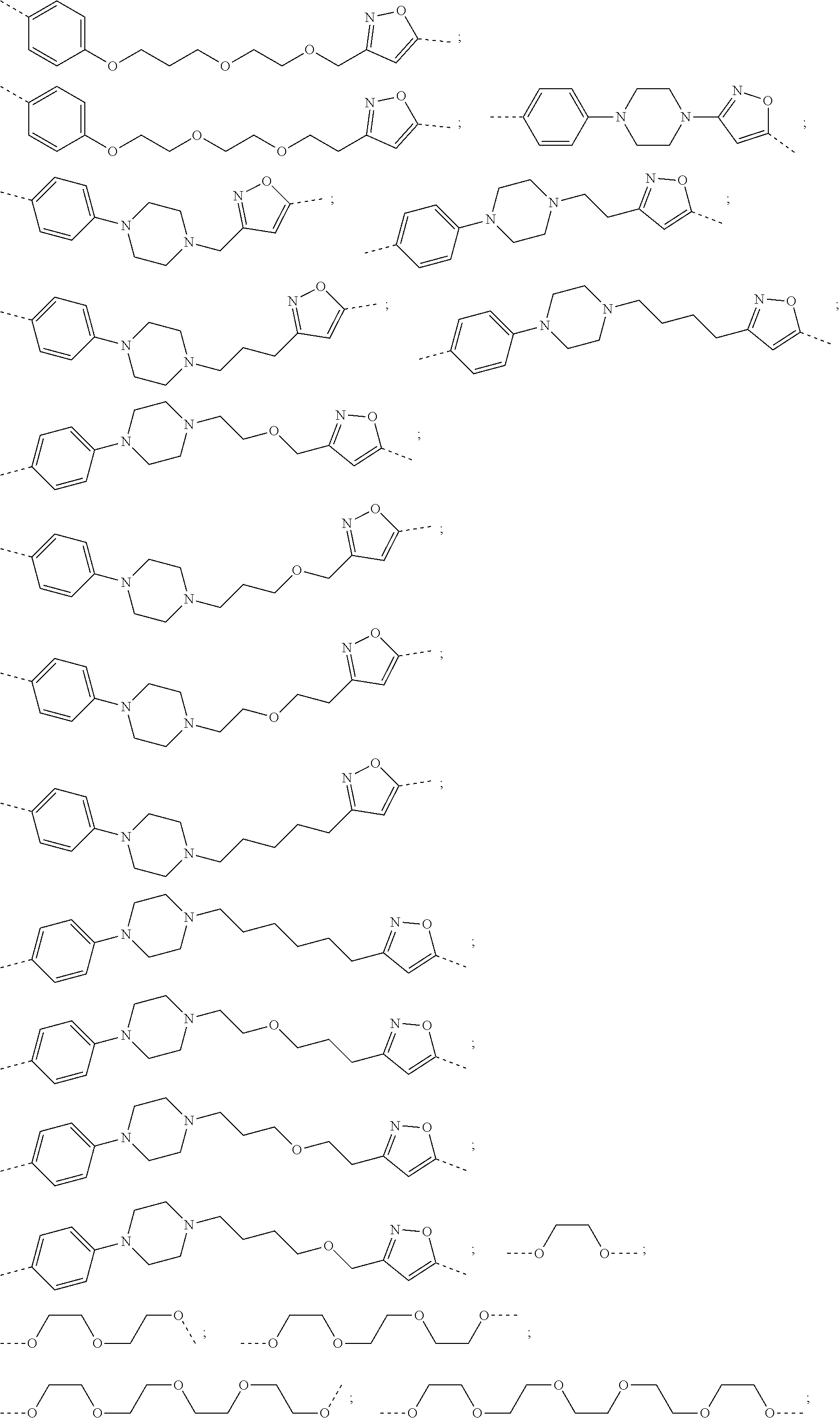

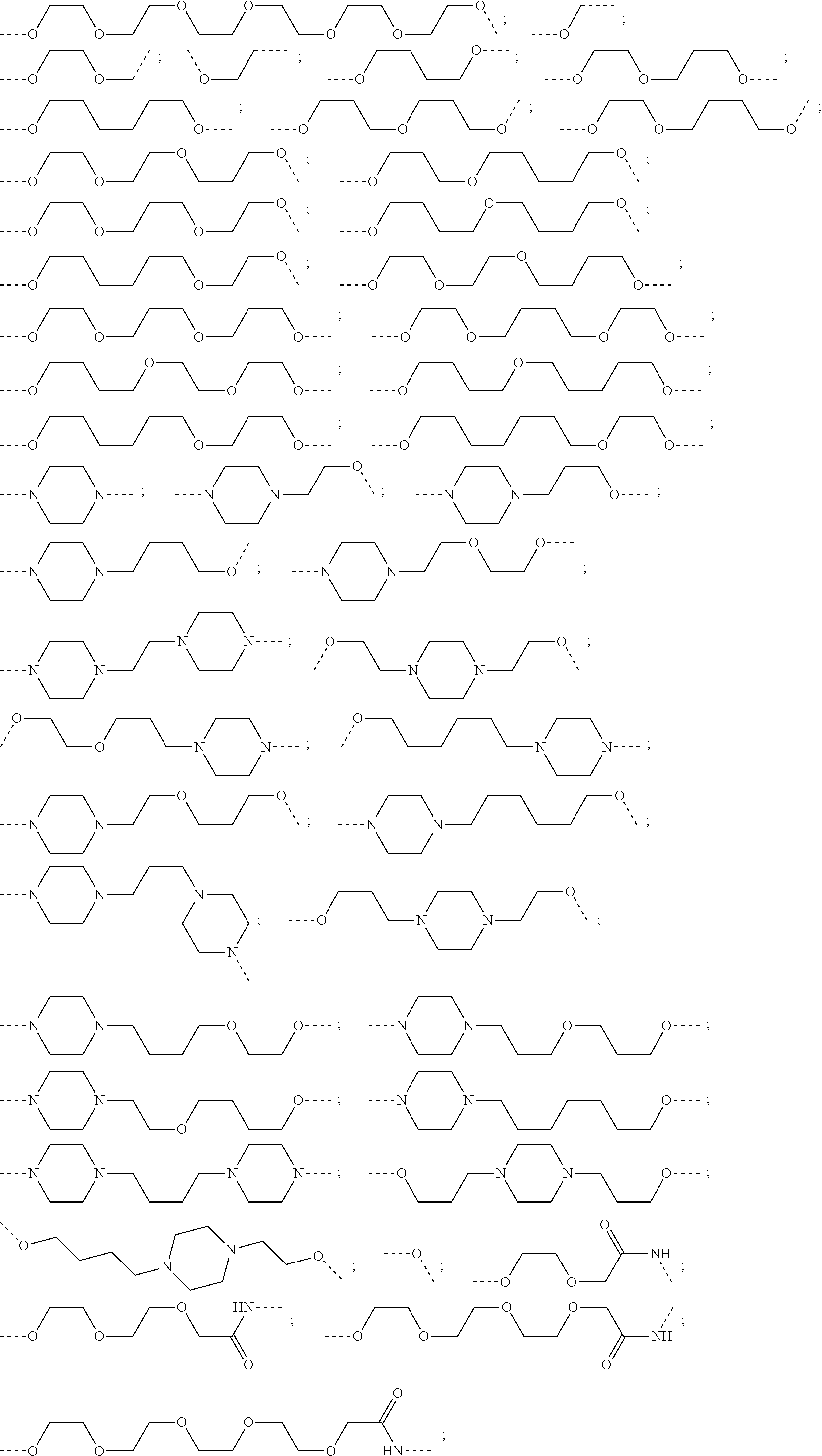

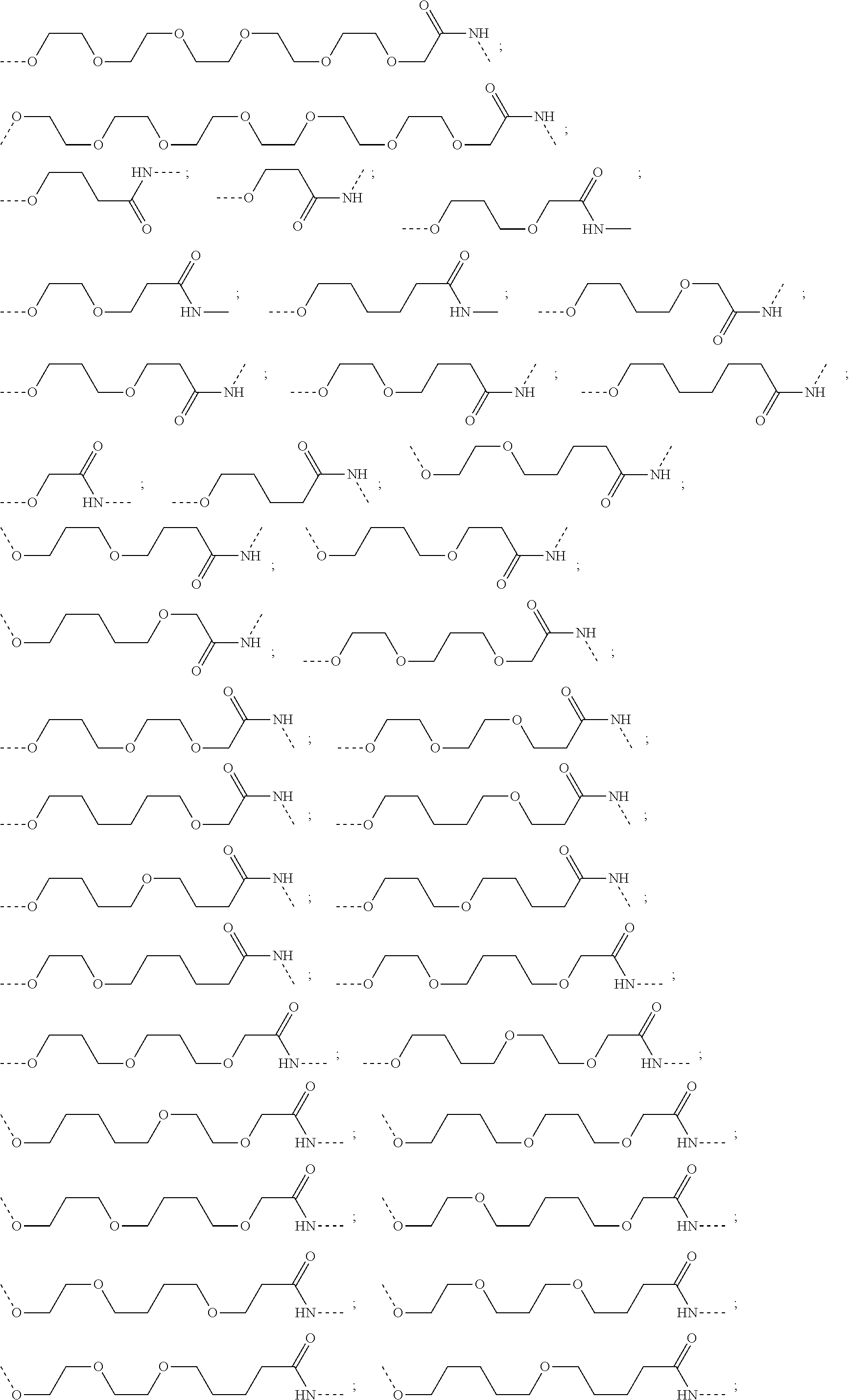

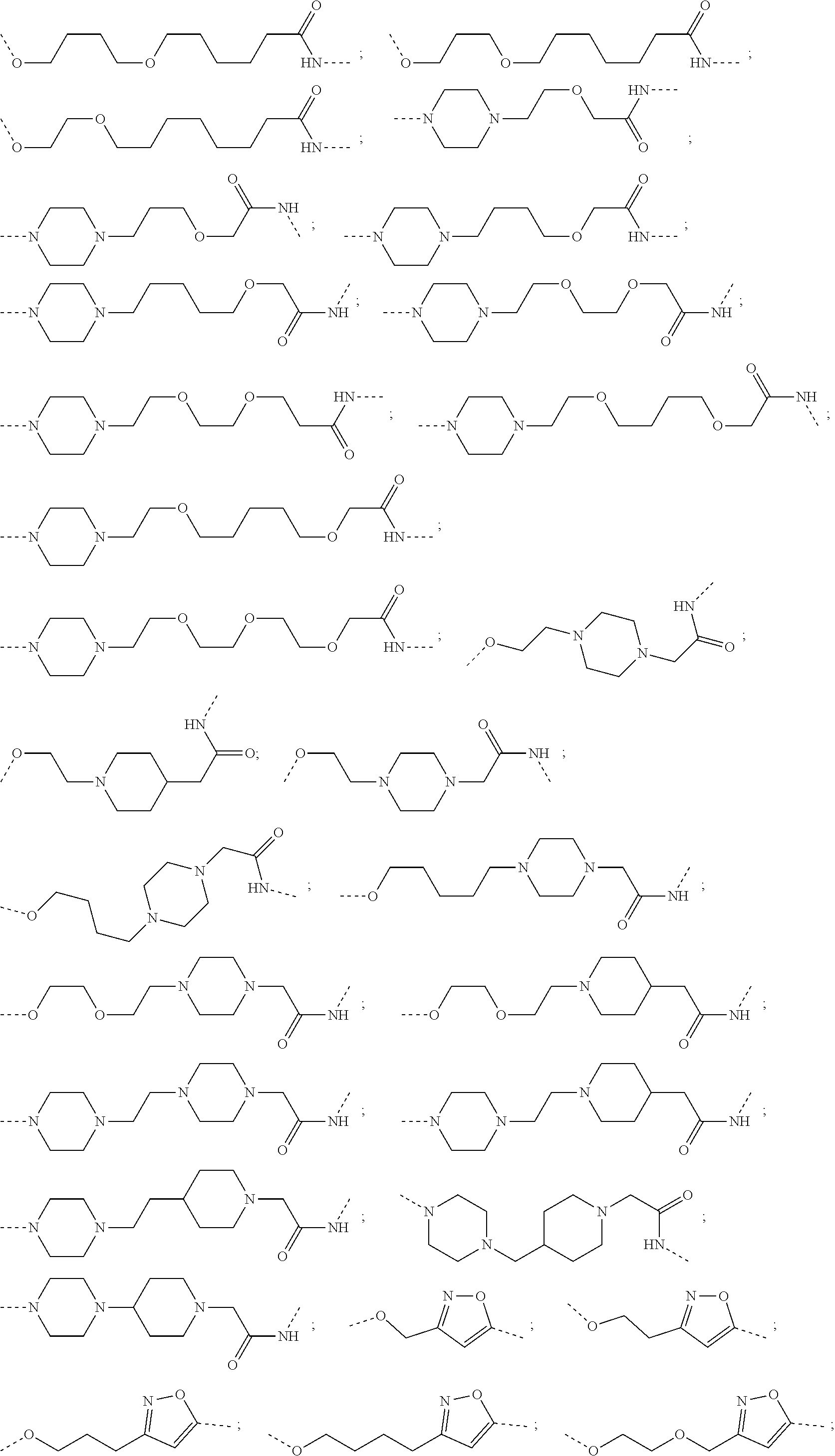

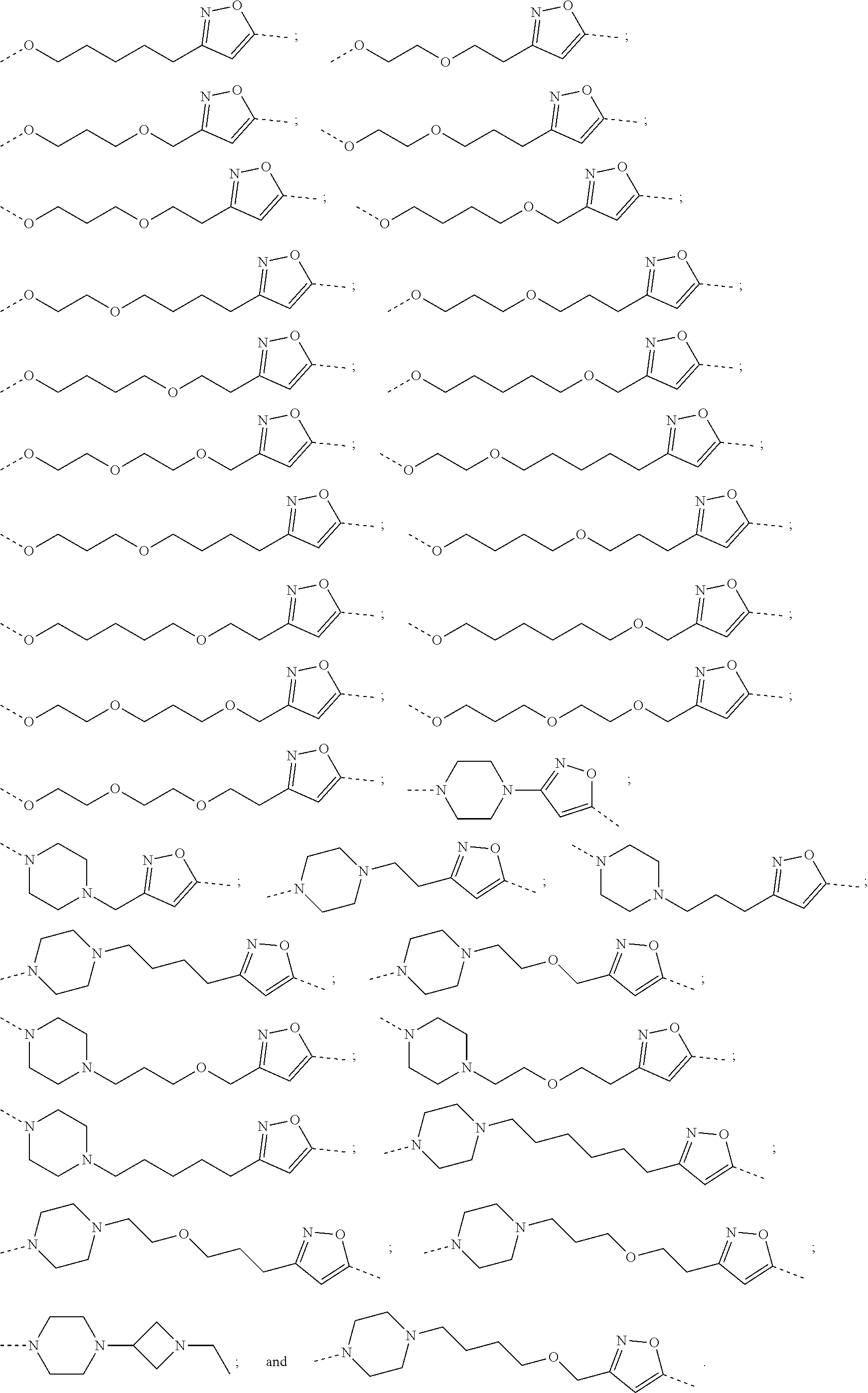

In any aspect or embodiment described herein, the linker (L) is a polyethylenoxy group optionally substituted with aryl or phenyl comprising from 1 to 10 ethylene glycol units.

In any aspect or embodiment described herein, the linker (L) comprises the following chemical structure:

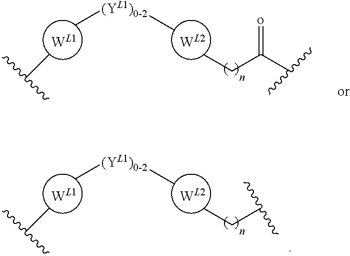

wherein:
W^{L1} and W¹² are each independently absent, a 4-8 membered ring with 0-4 heteroatoms, optionally substituted with RQ, each RQ is independently a H, halo, OH, CN, CF₃, optionally substituted linear or branched C₁-C₆ alkyl, optionally substituted linear or branched C₁-C₆ alkoxy, or 2 RQ groups taken together with the atom they are attached to, form a 4-8 membered ring system containing 0-4 heteroatoms;
Y^{L1} is each independently a bond, optionally substituted linear or branched C₁-C₆ alkyl and optionally one or more C atoms are replaced with O; or optionally substituted linear or branched C₁-C₆ alkoxy;
n is 0-10; and

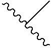

indicates the attachment point to the PTM or ULM moieties.

In any aspect or embodiment described herein, the linker (L) comprises the following chemical structure:

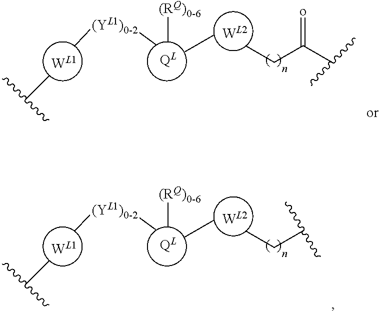

wherein:
W^{L1} and W^{L2} are each independently absent, aryl, heteroaryl, cyclic, heterocyclic, C₁₋₆ alkyl and optionally one or more C atoms are replaced with O, C₁₋₆ alkene and optionally one or more C atoms are replaced with O, C₁₋₆ alkyne and optionally one or more C atoms are replaced with O, bicyclic, biaryl, biheteroaryl, or biheterocyclic, each optionally substituted with R^{Q}, each R^{Q} is independently a H, halo, OH, CN, CF₃, hydroxyl, nitro, C≡CH, C₂₋₆ alkenyl, C₂₋₆ alkynyl, optionally substituted linear or branched C₁-C₆ alkyl, optionally substituted linear or branched C₁-C₆ alkoxy, optionally substituted linear or branched OC₁₋₃alkyl (e.g., optionally substituted by 1 or more —F), OH, NH₂, NR^{Y1}R^{Y2}, CN, or 2 R^{Q} groups taken together with the atom they are attached to, form a 4-8 membered ring system containing 0-4 heteroatoms;
Y^{L1} is each independently a bond, NR^{YL1}, O, S, NR^{YL2}, CR^{YL1}R^{YL2}, C═O, C═S, SO, SO₂, optionally substituted linear or branched C₁-C₆ alkyl and optionally one or more C atoms are replaced with O; optionally substituted linear or branched C₁-C₆ alkoxy;
Q^{L} is a 3-6 membered alicyclic or aromatic ring with 0-4 heteroatoms, optionally bridged, optionally substituted with 0-6 R^{Q}, each R^{Q} is independently H, optionally substituted linear or branched C₁₋₆ alkyl (e.g., optionally substituted by 1 or more halo or C₁₋₆ alkoxyl), or 2 R^{Q} groups taken together with the atom they are attached to, form a 3-8 membered ring system containing 0-2 heteroatoms;
R^{YL1}, R^{YL2} are each independently H, OH, optionally substituted linear or branched C₁₋₆ alkyl (e.g., optionally substituted by 1 or more halo or C₁₋₆ alkoxyl), or R¹, R² together with the atom they are attached to, form a 3-8 membered ring system containing 0-2 heteroatoms;
n is 0-10; and

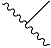

indicates the attachment point to the PTM or ULM moieties.

In any aspect or embodiment described herein, the linker (L) or the unit AL of linker (L) is selected from the group consisting of:

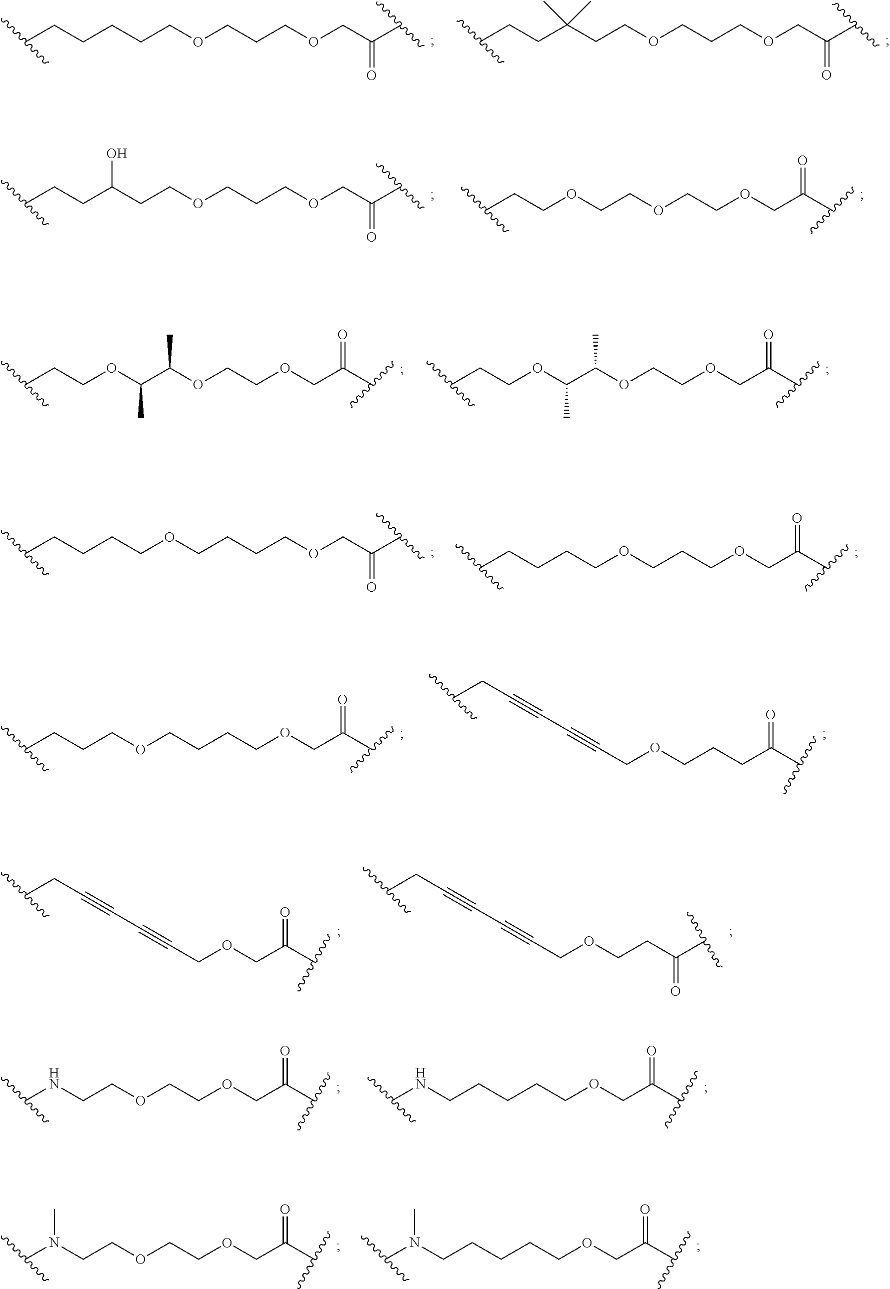

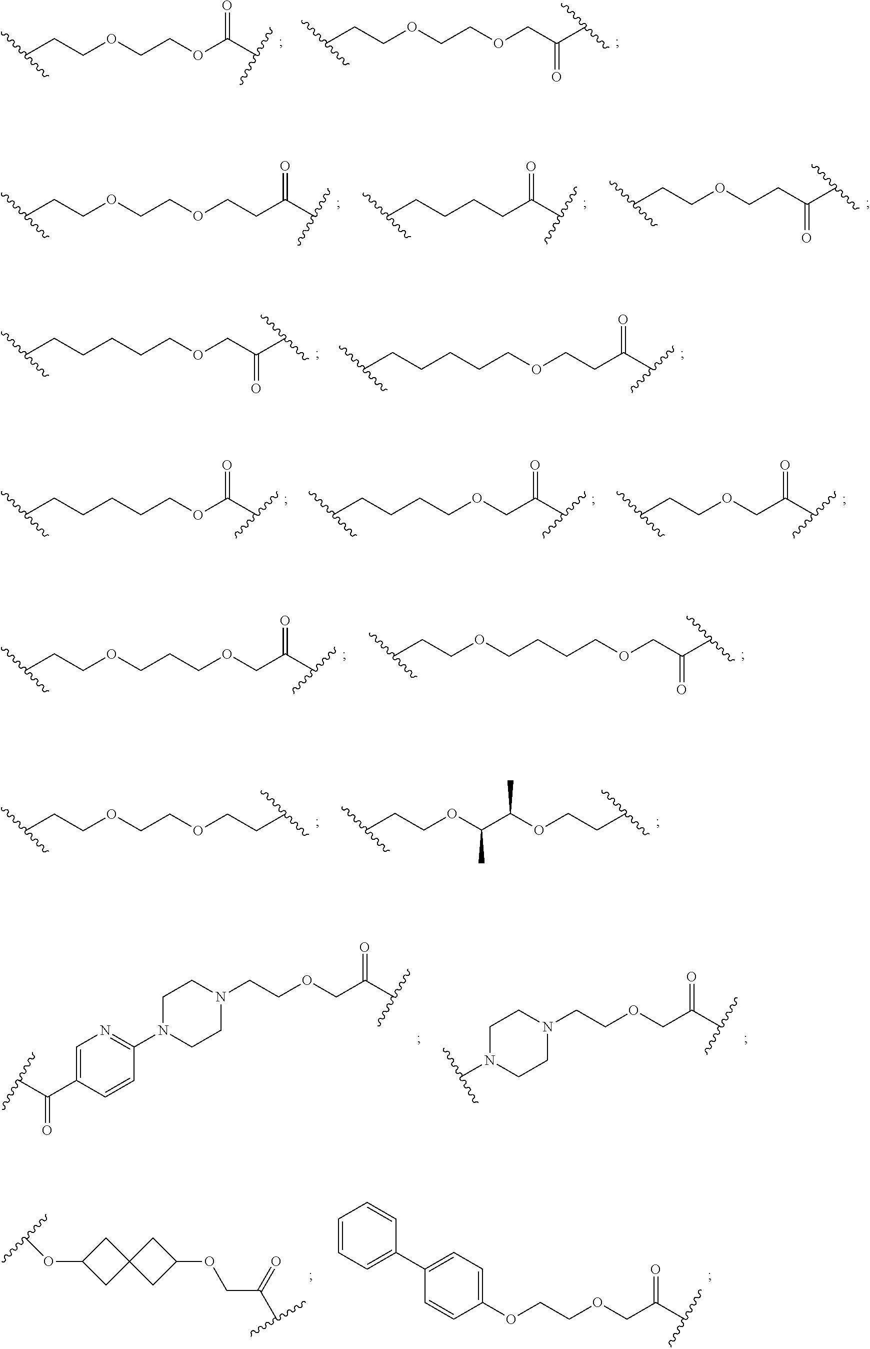

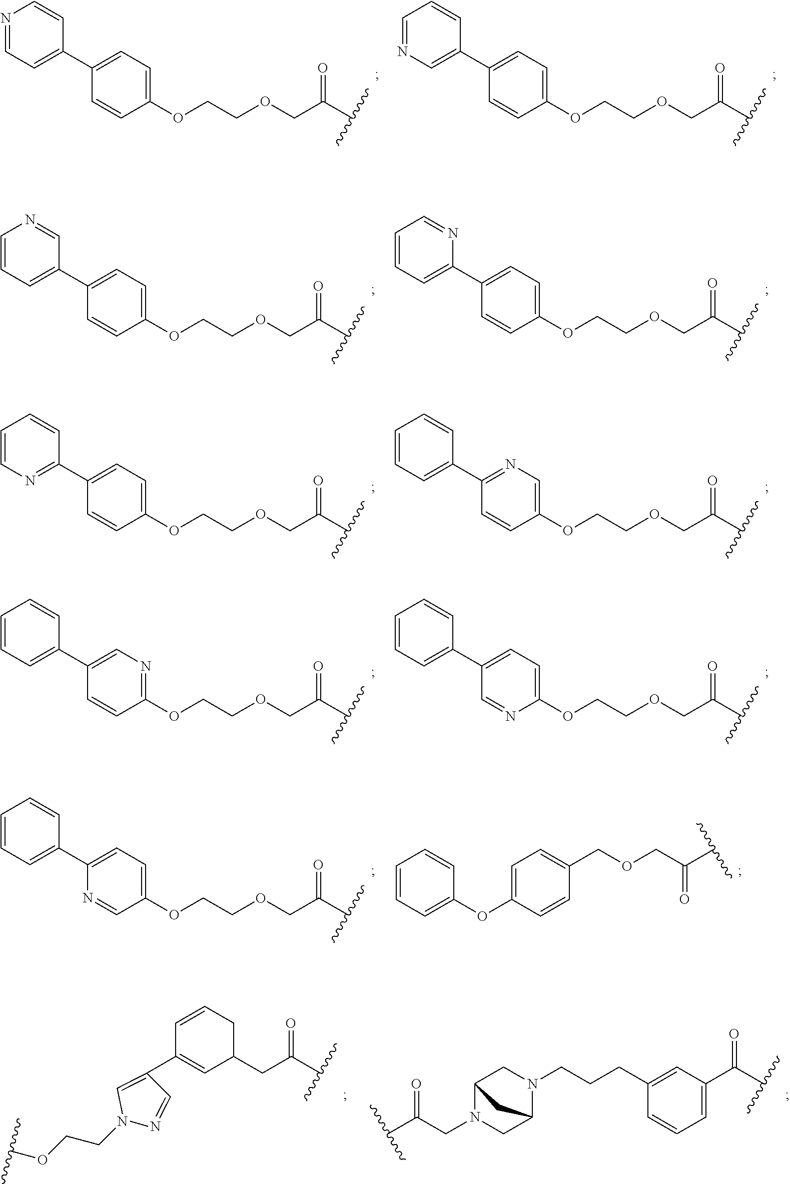

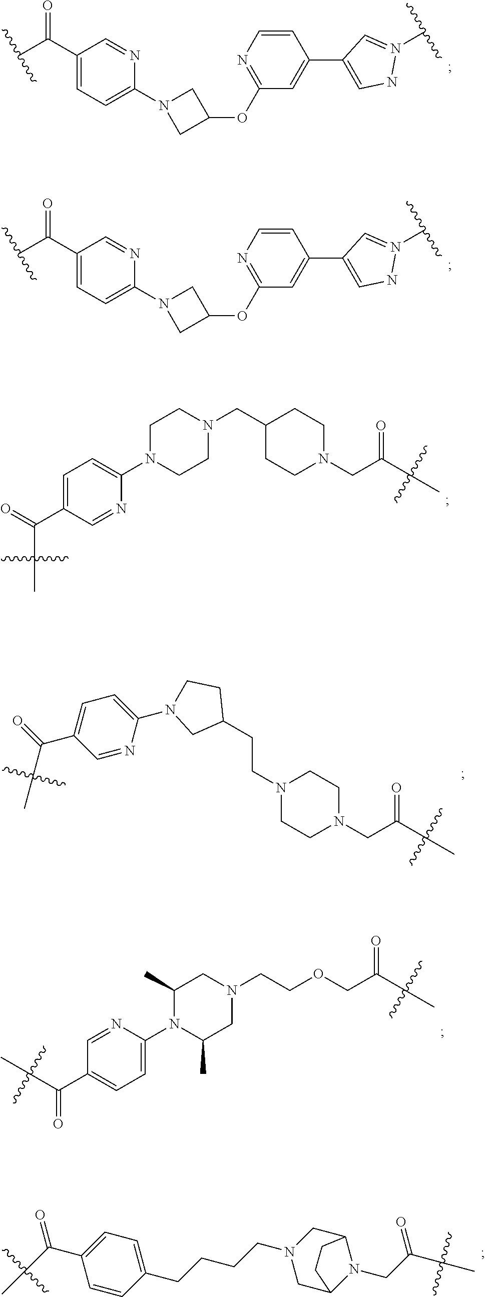

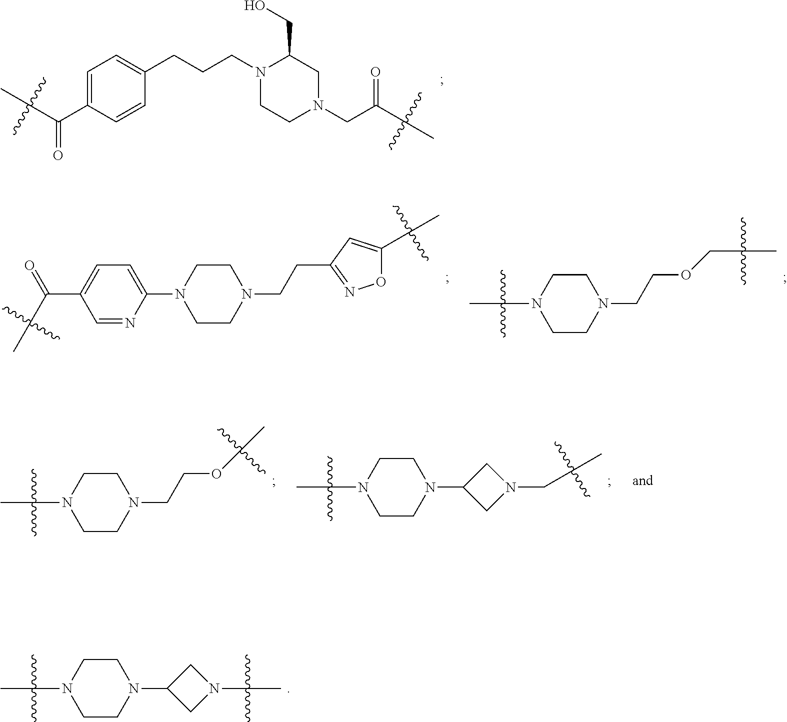 [SMILES: *C(=O)CN1CCN(CCCc2ccc(C(*)=O)cc2)[C@@H](CO)C1 ; *C(=O)c1ccc(N2CCN(CCc3cc(*)on3)CC2)nc1 ; *COCCN1CCN(*)CC1 ; *OCCN1CCN(*)CC1 ; *CN1CC(N2CCN(*)CC2)C1 ; *N1CCN(C2CN(*)C2)CC1]

In any aspect or embodiment described herein, the compound comprises multiple ULMs, multiple PTMs, multiple linkers or any combinations thereof.

In any aspect or embodiment described herein, the compound is selected from the group consisting of:

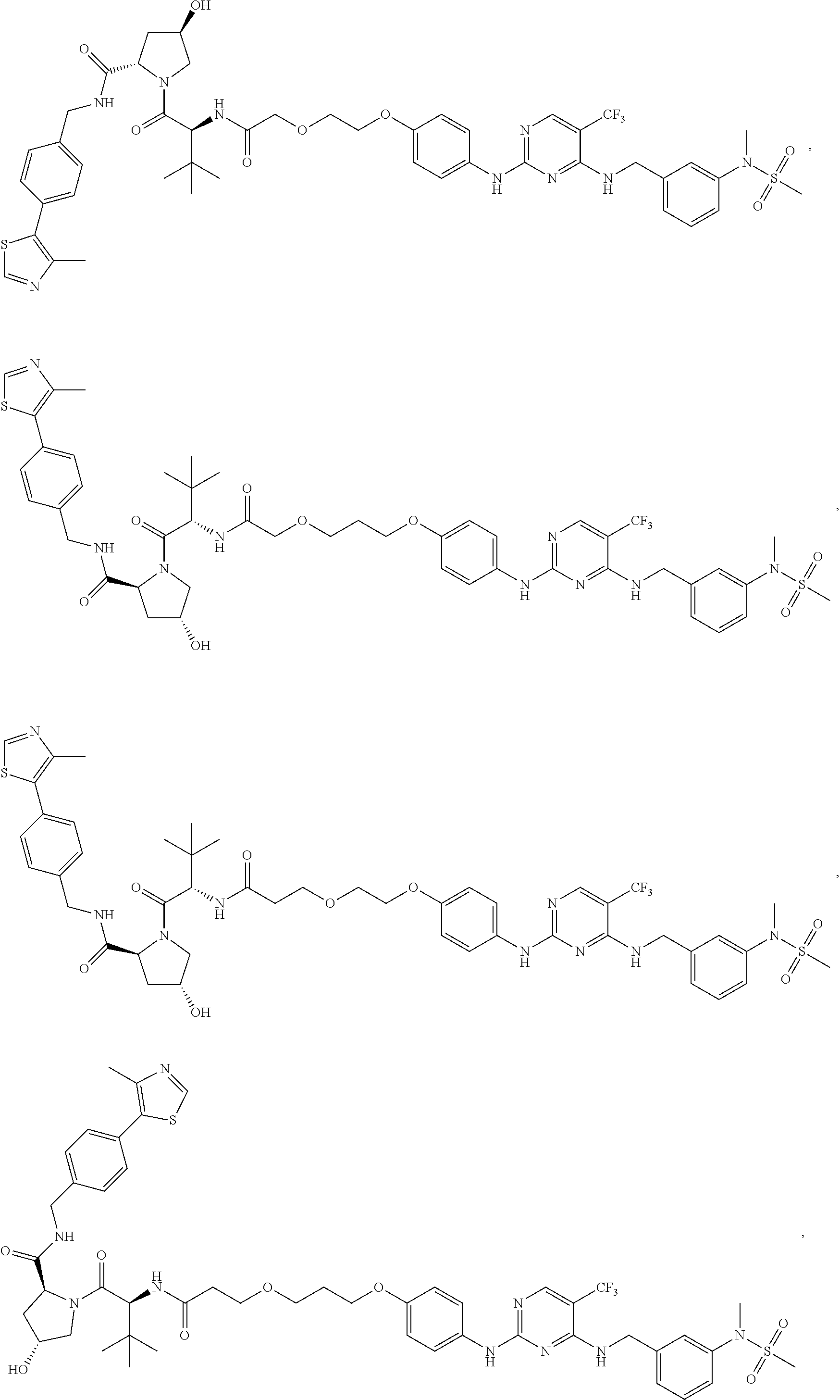

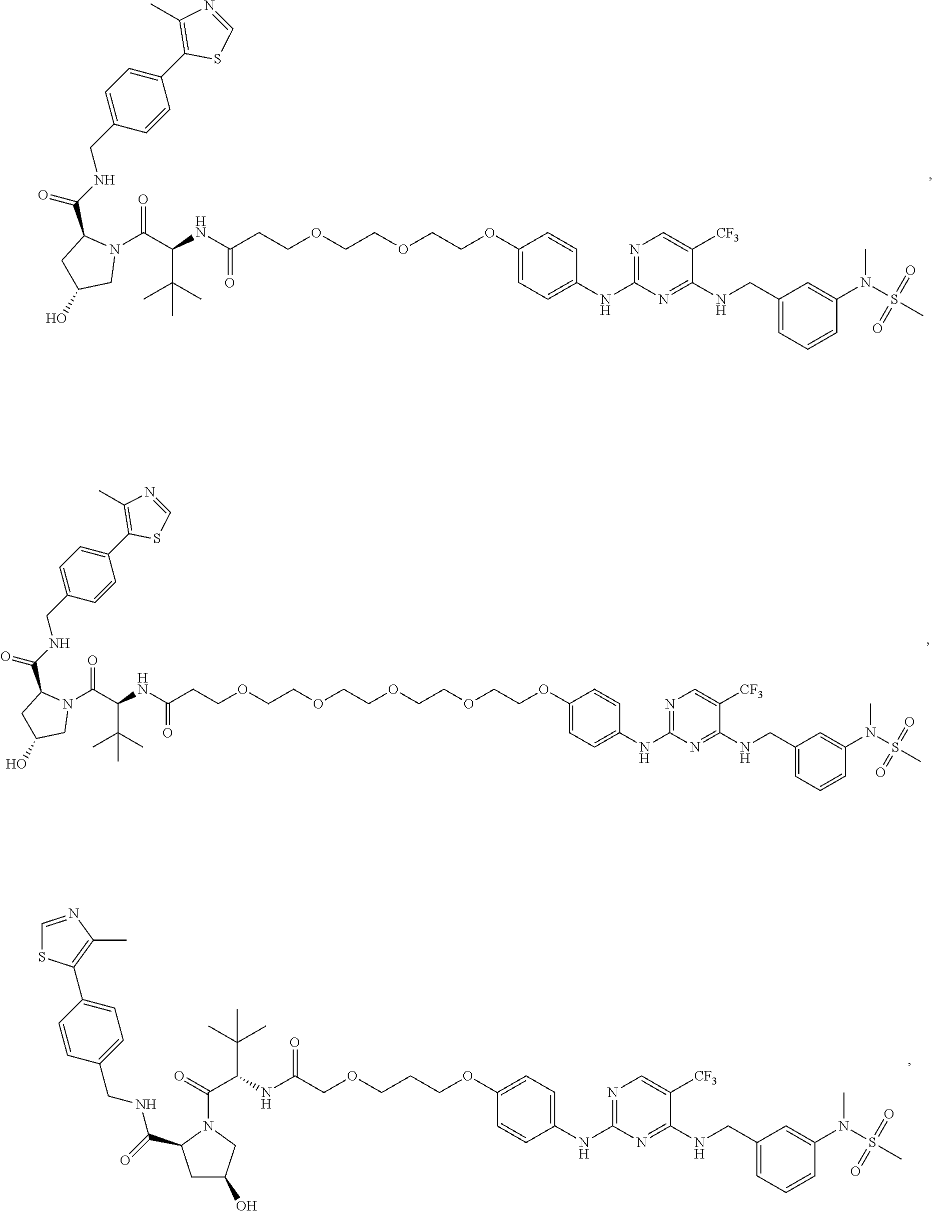 [SMILES: Cc1ncsc1-c1ccc(CNC(=O)[C@@H]2C[C@@H](O)CN2C(=O)[C@@H](NC(=O)CCOCCOCCOc2ccc(Nc3ncc(C(F)(F)F)c(NCc4cccc(N(C)S(C)(=O)=O)c4)n3)cc2)C(C)(C)C)cc1 ; Cc1ncsc1-c1ccc(CNC(=O)[C@@H]2C[C@@H](O)CN2C(=O)[C@@H](NC(=O)CCOCCOCCOCCOCCOc2ccc(Nc3ncc(C(F)(F)F)c(NCc4cccc(N(C)S(C)(=O)=O)c4)n3)cc2)C(C)(C)C)cc1 ; Cc1ncsc1-c1ccc(CNC(=O)[C@@H]2C[C@H](O)CN2C(=O)[C@@H](NC(=O)COCCCOc2ccc(Nc3ncc(C(F)(F)F)c(NCc4cccc(N(C)S(C)(=O)=O)c4)n3)cc2)C(C)(C)C)cc1]

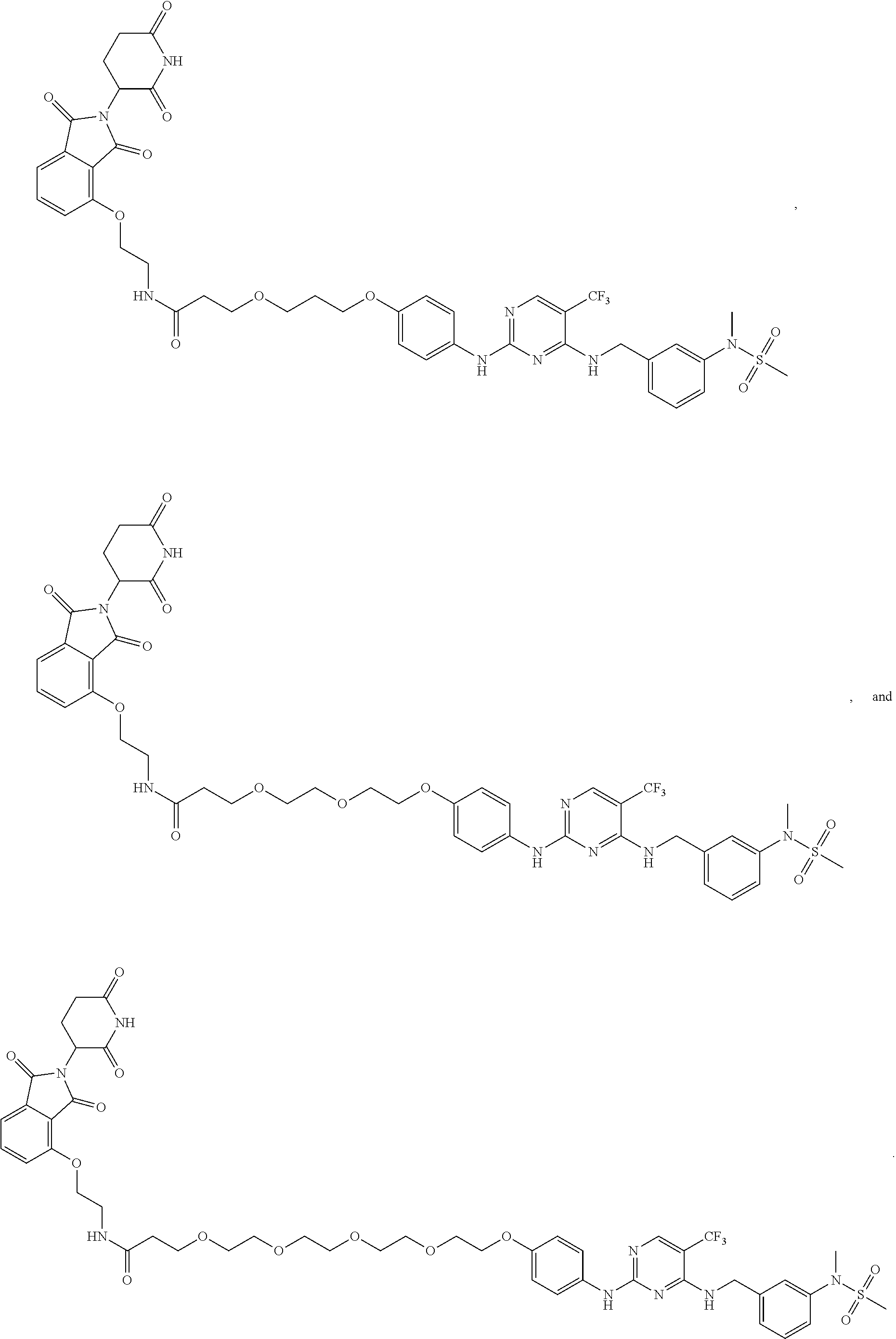 [SMILES: CN(c1cccc(CNc2nc(Nc3ccc(OCCCOCCC(=O)NCCOc4cccc5c4C(=O)N(C4CCC(=O)NC4=O)C5=O)cc3)ncc2C(F)(F)F)c1)S(C)(=O)=O ; CN(c1cccc(CNc2nc(Nc3ccc(OCCOCCOCCC(=O)NCCOc4cccc5c4C(=O)N(C4CCC(=O)NC4=O)C5=O)cc3)ncc2C(F)(F)F)c1)S(C)(=O)=O ; CN(c1cccc(CNc2nc(Nc3ccc(OCCOCCOCCOCCOCCC(=O)NCCOc4cccc5c4C(=O)N(C4CCC(=O)NC4=O)C5=O)cc3)ncc2C(F)(F)F)c1)S(C)(=O)=O]

Another aspect of the present disclosure provides a composition comprising an effective amount of a bifunctional compound of the present disclosure, and a pharmaceutically acceptable carrier.

In any aspect or embodiment described herein, the composition further comprises at least one of additional bioactive agent or another bifunctional compound of any of claims 1-26.

In any aspect or embodiment described herein, the additional bioactive agent is an anti-cancer agent.

A further aspect of the present disclosure provides a composition comprising a pharmaceutically acceptable carrier and an effective amount of at least one compound of the present disclosure for treating a disease or disorder in a subject, the method comprising administering the composition to a subject in need thereof, wherein the compound is effective in treating or ameliorating at least one symptom of the disease or disorder.

In any aspect or embodiment described herein, the disease or disorder is associated with at least one of one of accumulation, aggregation, overactivation, or combinations thereof, of FAKs.

In any aspect or embodiment described herein, the disease or disorder is cancer that is associated with the accumulation, aggregation, and/or overactivation of FAKs.

In any aspect or embodiment described herein, the disease or disorder is a solid tumor, carcinoma, adenocarcinoma, cystadenocarcinoma, endometriod carcinoma, mesothelioma, sarcoma, breast cancer, ovarian cancer, lung cancer, head and neck cancer, colorectal cancer, bladder cancer, uterine cancer, prostate cancer, squamous cell carcinoma, leukemia, glioblastoma and renal cancer.

In any aspect or embodiment described herein, the disease or disorder is ovarian cancer or breast cancer.

### EXAMPLES

### Synthetic Examples

Unless otherwise indicated, common reagents or materials were obtained from commercial sources and used without further purification. Fak kinase inhibitor defactinib was purchased from Selleckchem. Tetrahydrofuran (THF), Dimethylformamide (DMF) and Dichloromethane (CH₂Cl₂) were dried by a PureSolv™ solvent drying system. Flash column chromatography was performed using silica gel 60 (230-400 mesh). Analytical thin layer chromatography (TLC) was carried out on Merck silica gel plates with QF-254 indicator and visualized by UV or KMnO₄. Preparative TLC was performed on Analtech Silica Gel TLC plates (20×20 cm, 1000 μm). Magic base is a mixture of DCM, methanol and ammonium hydroxide (40% in water) (60:10:1). ¹H and ¹³C NMR spectra were recorded on an Agilent DD₂ 500 (500 MHz ¹H; 125 MHz ¹³C) or Agilent DD₂ 600 (600 MHz ¹H; 150 MHz ¹³C) or Agilent DD₂ 400 (400 MHz ¹H; 101 MHz ¹³C) spectrometer at room temperature. Chemical shifts were reported in ppm relative to the residual CDCl₃ (δ 7.26 ppm ¹H; δ 77.00 ppm ¹³C), CD₃OD (δ 4.87 ppm ¹H; δ 49.00 ppm ¹³C), or d⁶-DMSO (δ 2.50 ppm ¹H; δ 39.52 ppm ¹³C). NMR chemical shifts were expressed in ppm relative to internal solvent peaks, and coupling constants were measured in Hz. (bs=broad signal). Only peaks of the major rotamer are reported. Mass spectra were obtained using electrospray ionization (ESI) on a time of flight (TOF) mass spectrometer. Analytical HPLC analyses were carried out on 250×4.6 mm C-18 column using gradient conditions (10-100% B, flow rate=1.0 mL/min, 20 min). Preparative HPLC was carried out on 250×21.2 mm C-18 column using gradient conditions (10-100% B, flow rate=10.0 mL/min, 20 min). The eluents used were: solvent A (H₂O with 0.1% TFA) and solvent B (CH₃CN with 0.1% TFA).

### General Procedures (GPs)

GP1: Michael-Addition

To a solution of the diol (5.0 eq.) and tert-Butyl acrylate (1.0 eq.) in Acetonitrile (30 mL) was added Triton B (40 wt. % in water, 0.037 eq.) was added the reaction mixture was stirred for 72 hours at room temperature. The solvent was removed under reduced pressure and the crude product was purified by flash chromatography (12-100% ethyl acetate in hexanes) and the product visualized via KMnO₄ stain.

GP2: Iodination of Primary Alcohol

Triphenylphosphine (1.2 eq.), imidazole (1.2 eq.) and iodine (1.5 eq.) were dissolved in dry Tetrahydrofuran (20 mL) under inert atmospheric conditions (Argon). The primary alcohol (1.0 eq.) was added dropwise in dry Tetrahydrofuran (5 mL) and stirred at room temperature for 1.5 hours. The reaction mixture was filtered to remove the white precipitate and the solvent evaporated. The crude mixture was purified by flash chromatography (5-100% ethyl acetate in hexane) and the product visualized via KMnO₄ stain.

GP3: Linker Attachment to PTM

To a suspension of the PTM (1.0 eq.) and Cs₂CO₃ (3.0 eq.) in N,N-Dimethylformamide (3 mL) the iodinated linker (1.2 eq.) was added dropwise and the reaction mixture stirred for 16 hours at room temperature. After complete conversion (LC-MS) the reaction mixture was poured onto ethyl acetate (50 mL) and extracted with a mixture of water and sat. NaHCO₃ (1:5, v/v, 3×50 mL). The combined organic layers were washed with brine and dried over MgSO₄. The solvent was removed under reduced pressure and the product purified by PTLC (DCM/magic base 3:1).

GP4: tert-Butyl Deprotection

The tert-butyl protected compound was dissolved in 50% TFA in DCM (10 mL) and stirred at room temperature for 2 hours. Afterwards, the solvents were removed under reduced pressure and the deprotected product used without further purification.

GP5: HATU Coupling E3 Recruiting Ligand

To a solution of the free acid (1.0 eq.), and the primary amine (1.2 eq.) in N,N-Dimethylformamide (2 mL) was added 0-(7-Azabenzotriazol-1-yl)-N,N,N′,N′-tetramethyluronium hexafluorophosphate (HATU) (2.0 eq.) and N,N-Diisopropylethylamine (50 eq.) and the reaction mixture stirred at room temperature for 16 hours. The reaction mixture was poured onto ethyl acetate (50 mL) and extracted with a mixture of water and sat. NaHCO₃ (1:5, v/v, 3×50 mL). The combined organic layers were washed with brine and dried over MgSO₄. The solvent was removed under reduced pressure and the product purified by PTLC (DCM/magic base 2:1).

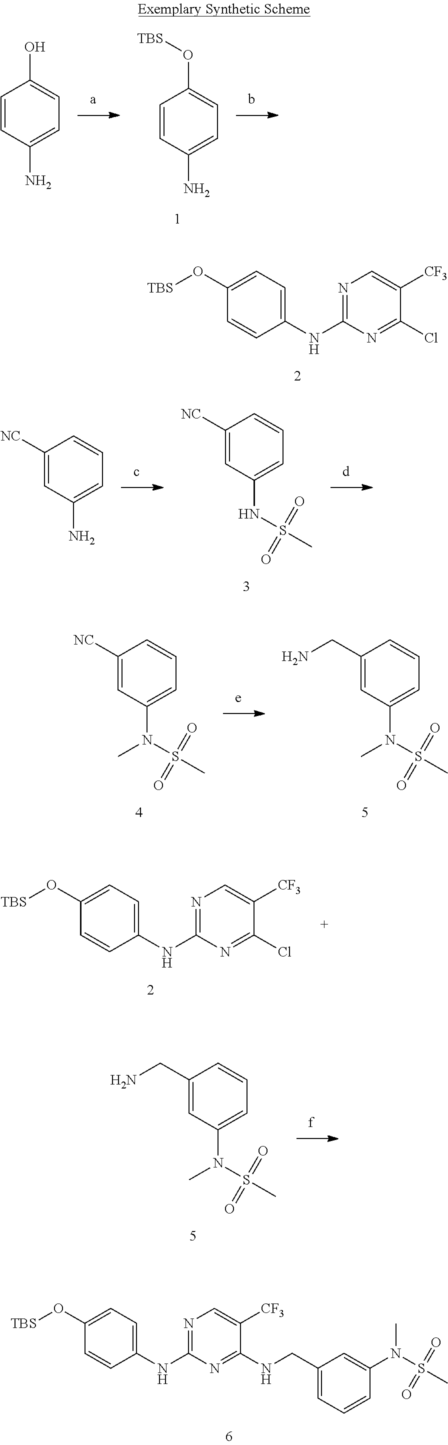

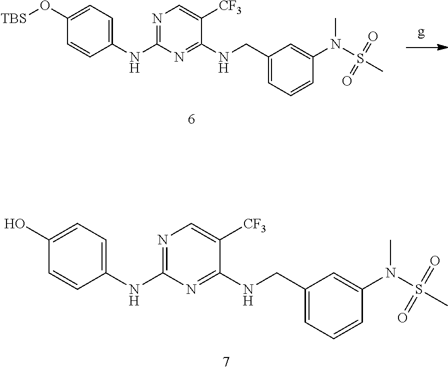

Reagents/Solvents/Conditions.

a) Imidazole, TBS-Cl, DMF, room temperature; b) 2,4-dichloro-5-(trifluoromethyl)pyrimidine, Zinc bromide, TEA, tBuOH/DCE (1:1, v/v), room temperature; c) Mesyl chloride, pyridine, DCM, room temperature; d) Iodomethane, K₂CO₃, DMF, room temperature; e) Raney Nickel, 7N NH₃ in Methanol, room temperature; f) DIEA, 1 tBuOH/DCE (1:1, v/v), 80° C.; g) TBAF, THF, room temperature.

### Synthesis of 4-((tert-butyldimethylsilyl)oxy)aniline (1)

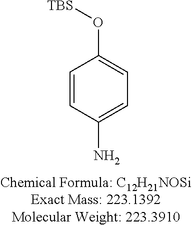

To a solution of 4-aminophenol (1000 mg, 9.2 mmol, 1.0 eq.) in N,N-Dimethylformamide (20 mL), Imidazole (1872 mg, 27.5 mmol, 3.0 eq.) and tert-butyl-chloro-dimethyl-silane (1657 mg, 11.00 mmol, 1.2 eq.) were added and the reaction mixture was stirred at room temperature for 20 hours. The reaction mixture was poured into water (100 mL) and was extracted with ether (3×100 mL). The combined organic layers were washed with brine, dried over MgSO₄ and concentrated under reduced pressure. The crude product was purified via flash chromatography (PE/EA 10:1->5:1) to yield 1222 mg (60%) of the desired product. MS: calc. [M+H]⁺ for C₁₂H₂₂NOSi⁺=224.1465 [M+H]⁺; found=224.1389 [M+H]⁺

¹H NMR (400 MHz, CDCl₃): δ=6.67-6.62 (m, 2H), 6.59-6.54 (m, 2H), 0.96 (s, 9H), 0.14 (s, 6H). ¹³C NMR (101 MHz, CDCl₃): δ=148.3, 140.4, 120.8, 116.4, 25.9, 18.3, -4.4.

### Synthesis of N-(4-((tert-butyldimethylsilyl)oxy)phenyl)-4-chloro-5-(trifluoromethyl)pyrimidin-2-amine (2)

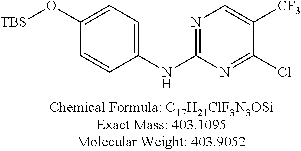

A solution of 2,4-dichloro-5-(trifluoromethyl)pyrimidine (0.14 ml, 1.1 mmol, 1.2 eq.) in 1:1 ^{t}BuOH/DCE (4 mL) was cooled to 0° C. Solid Zinc Bromide (605 mg, 2.7 mmol, 3.0 eq.) was added and the reaction mixture was stirred at 0° C. for 30 minutes. The reaction was maintained at 0° C. and treated first with 1 (200 mg, 0.9 mmol, 1.0 eq.) followed by TEA (0.28 mL, 1.97 mmol, 2.2 eq.). The resultant white mixture was allowed to warm to room temperature and stirred for 20 hours. The solvent was removed under reduces pressure and the crude product purified via flash chromatography (PE/EA 50:1->25:1) to yield the final product in 97% (324 mg).

MS: calc. [M+H]⁺ for C₁₇H₂₂ClF₃N₃OSi⁺=404.1167 [M+H]⁺; found=404.1179 [M+H]⁺

¹H NMR (400 MHz, CDCl₃): δ=8.51 (s, 1H), 7.43-7.38 (m, 2H), 6.86-6.82 (m, 2H), 0.98 (s, 9H), 0.20 (s, 6H). ¹⁹F NMR (376 MHz, DMSO)=δ −60.02. ¹³C NMR (101 MHz, CDCl₃): δ=160.8, 157.5, 152.9, 130.9, 124.3, 124.2, 122.5, 121.5, 120.6, 25.8, 18.3, -4.3.

### Synthesis of N-(3-cyanophenyl)methanesulfonamide (3)

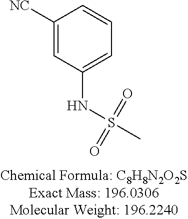

A solution of 3-aminobenzonitrile (2000 mg, 16.9 mmol, 1.0 eq.) and Pyridine (1.5 mL, 18.6 mmol, 1.1 eq.) in DCM (50 mL) was cooled to 0° C. and Mesyl chloride (1.30 mL, 16.9 mmol, 1.0 eq.) was added dropwise. The reaction mixture was allowed to warm to room temperature and was stirred at for 4 hours at room temperature. The solvent was removed under educed pressure, the precipitate dissolved in 1N HCl (50 mL) and extracted with ethyl acetate (3×50 mL). The combined organic layers were washed with brine, dried over MgSO₄ and the solvent removed under reduced pressure to yield 3300 mg of the desired product (99%).

MS: calc. [M+H]⁺ for C₈H₉N₂Q₂S⁺=197.0379 [M+H]⁺; found=197.0356 [M+H]⁺

¹H NMR (400 MHz, Methanol-D₄): δ=7.60-7.55 (m, 1H), 7.55-7.51 (m, 2H), 7.50-7.45 (m, 1H), 3.03 (s, 3H). ¹³C NMR (101 MHz, Methanol-D₄): δ=140.8, 131.7, 128.6, 125.18, 123.5, 119.2, 114.4, 39.8.

### Synthesis of N-(3-cyanophenyl)-N-methylmethanesulfonamide (4)

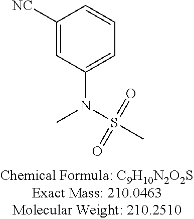

To a mixture of 3 (1.5 g, 7.6 mmol, 1.0 eq.) and K₂CO₃ (2.1 g, 15.4 mmol, 2.0 eq.) in N,N-Dimethylformamide (15.0 mL) was added CH₃I (0.6 mL, 9.2 mmol, 1.2 eq.) and the reaction mixture was stirred at room temperature for 16 hours. The suspension was diluted with ethyl acetate (50 mL) and washed with a mixture of water and sat. NaHCO₃ (3×1:2 (v/v), 60 mL). The washing with brine, the organic phase was dried over MgSO₄ and concentrated under reduced pressure. The crude product was triturated with hexane to yield 1521 mg (95%) of the desired product.

MS: calc. [M+H]⁺ for C₉H₁₀N₂O₂S⁺=210.0457 [M+H]⁺; found=210.0447 [M+H]⁺
¹H NMR (400 MHz, CDCl₃): δ=7.72-7.62 (m, 2H), 7.62-7.56 (m, 1H), 7.54-7.48 (m, 1H), 3.35 (s, 3H), 2.87 (s, 3H). ¹³C NMR (101 MHz, CDCl₃): δ=142.5, 130.7, 130.5, 130.4, 128.8, 118.0, 113.7, 43.0, 35.9.

### Synthesis of N-(3-(aminomethyl)phenyl)-N-methylmethanesulfonamide (5)

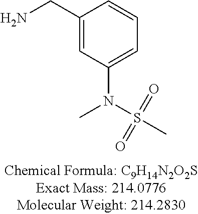

1.5 g of 4 (7.2 mmol, 1.0 eq.) were dissolved in 7N NH₃ Methanol (15 ml) in a Schlenkflask. Under Argon Raney Nickel (50% slurry, 2 mL) was added and the reaction placed under Hydrogen atmosphere and stirred for 16 h at room temperature. The mixture was filtered over celite and the solved removed under reduced pressure. The product was triturated in a mixture of ethyl acetate (20 mL) and 2M HCl in ether (15 mL) to yield 1420 mg of the desired product (92%).

MS: calc. [M+H]⁺ for C₉H₁₅N₂S⁺=215.0849 [M+H]⁺; found=215.0865 [M+H]⁺

¹H NMR (400 MHz, Methanol-D₄): δ=7.59-7.56 (m, 1H), 7.52-7.49 (m, 2H), 7.45-7.40 (m, 1H), 4.16 (s, 2H), 3.33 (s, 3H), 2.93 (s, 3H). ¹³C NMR (101 MHz, Methanol-D₄): δ=143.8, 135.6, 131.1, 128.9, 128.3, 128.1, 43.9, 38.5, 35.6.

### Synthesis of N-(3-(((2-((4-((tert-butyldimethylsilyl)oxy)phenyl)amino)-5-(trifluoromethyl)pyrimidin-4-yl)amino)methyl)phenyl)-N-methylmethanesulfonamide (6)

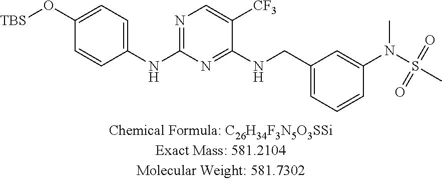

A suspension of 5 (1592 mg, 7.5 mmol, 2.5 eq.), 2 (1200 mg, 3.0 mmol, 1.0 eq.), and DIEA (2.54 mL, 14.9 mmol, 5.0 eq.) in 1:1 (v/v) DCE/^{t}BuOH was heated to 80° C. for 16 hours. The reaction mixture was allowed to cool to room temperature and it was stirred for another 20 hours. Afterwards, the solvents were removed under reduced pressure and the crude product purified via flash chromatography (12%-100% ethyl acetate in hexane) to yield 1667 mg (97%) of the desired product.

MS: calc. [M+H]⁺ for C₂₆H₃₅F₃N₅O₃SSi⁺=582.2176 [M+H]⁺; found=582.2283 [M+H]⁺

¹H NMR (400 MHz, CDCl₃): δ=8.19-8.14 (m, 1H), 7.39-7.26 (m, 5H), 7.22 (dt, J=7.6, 1.4 Hz, 1H), 6.76 (dd, J=9.1, 2.9 Hz, 2H), 4.70 (d, J=5.7 Hz, 2H), 3.27 (s, 3H), 2.78 (s, 3H), 0.98 (s, 9H), 0.18 (s, 6H). ¹⁹F NMR (376 MHz, CDCl₃) δ=−61.15. ¹³C NMR (101 MHz, CDCl₃): δ=160.7, 158.7, 154.6, 151.7, 141.7, 139.6, 132.2, 130.0, 129.6, 126.2, 125.6, 124.6, 124.1, 122.1, 121.8, 120.2, 120.1, 119.4, 44.5, 37.9, 35.2, 25.7, 18.2, -4.5.

### Synthesis of N-(3-(((2-((4-hydroxyphenyl)amino)-5-(trifluoromethyl)pyrimidin-4-yl)amino)methyl)phenyl)-N-methylmethanesulfonamide (7)

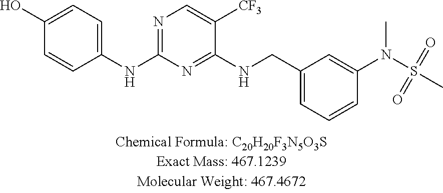 [SMILES: CN(c1cccc(CNc2nc(Nc3ccc(O)cc3)ncc2C(F)(F)F)c1)S(C)(=O)=O]

1670 mg of 7 (2.87 mmol, 1.0 eq.) were dissolved in THF (5 mL) and 1M TBAF in THF (5.7 mL, 2.0 eq.) were added and the reaction mixture stirred for 16 h at room temperature. The solvent was removed, the crude product taken up in water/ethyl acetate 1:1 (60 mL, v/v) and extracted with ethyl acetate (2×50 mL). The combined organic layers were washed with brine, dried over MgSO₄ and concentrated under reduced pressure. The crude product was purified by flash chromatography (30-100% ethyl acetate in Hexane) to yield 1252 mg (94%) of the desired product.

MS: calc. [M+H]⁺ for C₂₀H₂₁F₃N₅O₃S⁺=468.1312 [M+H]⁺; found=468.1322 [M+H]⁺

¹H NMR (400 MHz, CDCl₃): δ=8.16 (s, 1H), 7.41-7.30 (m, 2H), 7.26-7.13 (m, 4H), 6.76-6.70 (m, 2H), 4.67 (d, J=5.8 Hz, 2H), 3.27 (s, 3H), 2.81 (s, 3H). ¹⁹F NMR (376 MHz, CDCl₃) δ=−61.14. ¹³C NMR (101 MHz, CDCl₃): δ=161.1, 159.0, 154.8, 152.4, 141.8, 139.9, 131.5, 129.8, 126.2, 126.1, 124.8, 123.7, 123.0, 115.7, 44.7, 38.2, 35.5.

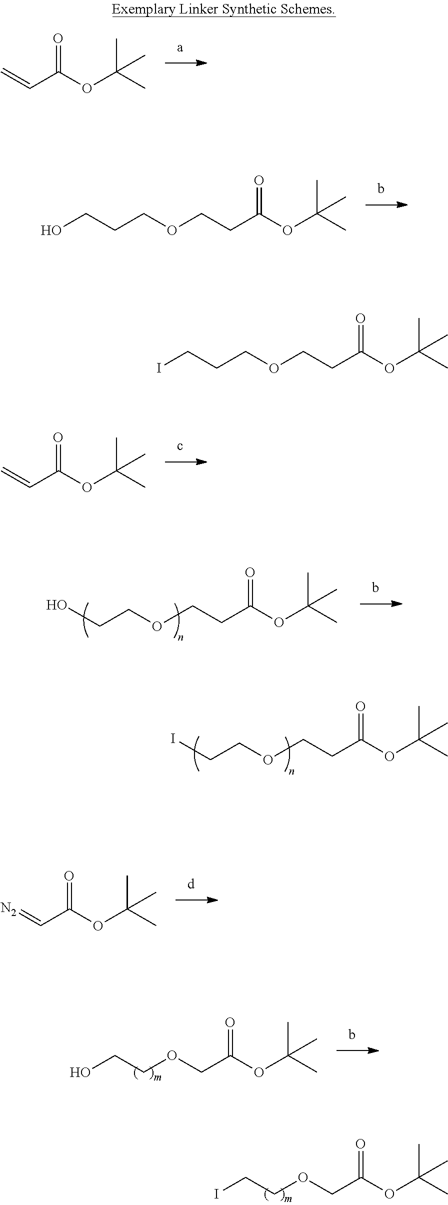

Reagents/Solvents/Conditions:

a) Triton B, 1,3 propane diol, ACN, room temperature; b) PPh₃, I₂, imidazole, THF, room temperature; c) Triton B, diol, ACN, room temperature; d) Rh(II) acetate, DCM, room temperature; n=1, 2, 4; m=1, 2.

### Synthesis of tert-butyl 3-(3-hydroxypropoxy)propanoate (8)

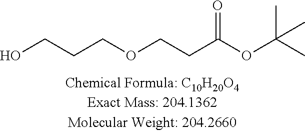

tert-Butyl acrylate (4000 mg) was reacted with propane-1,3-diol according to GP1 and yielded compound 8 in 71% (4468 mg).

¹H NMR (400 MHz, CDCl₃): δ=3.75 (t, J=5.6 Hz, 2H), 3.67 (t, J=6.2 Hz, 2H), 3.63 (t, J=5.7 Hz, 2H), 2.48 (t, J=6.2 Hz, 2H), 1.81 (p, J=5.6 Hz, 2H), 1.45 (s, 9H). ¹³C NMR (101 MHz, CDCl₃): δ=171.2, 80.9, 70.3, 66.9, 62.0, 36.4, 32.1, 28.2.

### Synthesis of tert-butyl 3-(2-hydroxyethoxy)propanoate (9)

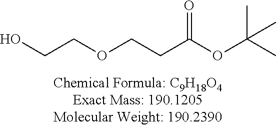

tert-Butyl acrylate (2000 mg) was reacted with ethylene glycol according to GP1 and yielded compound 9 in 35% (1035 mg).

¹H NMR (400 MHz, CDCl₃): δ=3.76-3.68 (m, 4H), 3.59-3.55 (m, 2H), 2.50 (t, J=6.1 Hz, 2H), 1.45 (s, 9H). ¹³C NMR (101 MHz, CDCl₃): δ=171.4, 81.0, 72.0, 66.5, 61.8, 36.3, 28.2.

### Synthesis of tert-butyl 3-(2-(2-hydroxyethoxy)ethoxy)propanoate (10)

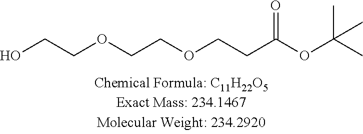 [SMILES: CC(C)(C)OC(=O)CCOCCOCCO]

tert-Butyl acrylate (2000 mg) was reacted with diethylene glycol according to GP1 and yielded compound 10 in 9% (477 mg).

¹H NMR (400 MHz, CDCl₃): δ=3.72 (t, J=6.4 Hz, 4H), 3.66-3.56 (m, 6H), 2.50 (t, J=6.4 Hz, 2H), 1.44 (s, 9H). ¹³C NMR (101 MHz, CDCl₃): δ=171.1, 80.8, 72.6, 70.5, 67.0, 61.9, 36.3, 28.2.

### of tert-butyl 1-hydroxy-3,6,9,12-tetraoxapentadecan-15-oate (11)

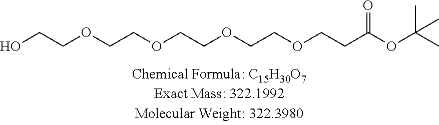 [SMILES: CC(C)(C)OC(=O)CCOCCOCCOCCOCCO]

tert-Butyl acrylate (1050 mg) was reacted with tetraethylene glycol according to GP1 and yielded compound 11 in 60% (1556 mg).

¹H NMR (400 MHz, CDCl₃): δ=3.76-3.54 (m, 18H), 2.50 (t, J=6.6 Hz, 2H), 1.44 (s, 9H). ¹³C

¹H NMR (101 MHz, CDCl₃): δ=171.04, 80.66, 72.67, 70.76, 70.72, 70.71, 70.63, 70.50, 70.49, 67.03, 61.90, 36.38, 28.23.

### Synthesis of tert-butyl 2-(2-hydroxyethoxy)acetate (12)

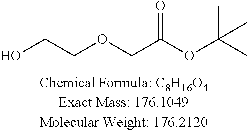

A solution of ethylene glycol (1.3 mL, 23.5 mmol, 5.0 eq.) and Rhodium(II) acetate dimer (31.1 mg, 0.1 mmol, 15 mol %) in DCM (15 mL) was stirred at room temperature under Argon. A solution of tert-butyl 2-diazoacetate (0.65 mL, 4.7 mmol, 1.0 eq.) in DCM (5 mL) was added dropwise. After the addition was complete the reaction mixture was stirred at room temperature for 16 hours. The mixture was concentrated under reduced pressure and the crude product purified by flash column chromatography (20% -100% ethyl acetate in hexane) to yield 552 mg (67%) of the desired product.

¹H NMR (400 MHz, CDCl₃): δ=4.01 (s, 2H), 3.73 (dt, J=5.3, 2.9 Hz, 2H), 3.69-3.59 (m, 2H), 1.48 (s, 9H). ¹³C NMR (101 MHz, CDCl₃): δ=170.6, 82.3, 73.7, 67.0, 61.8, 28.2.

### Synthesis of tert-butyl 2-(3-hydroxypropoxy)acetate (13)

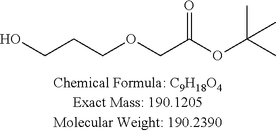

A solution of propane-1,3-diol (1.7 mL, 23.5 mmol, 5.0 eq.) and Rhodium(II) acetate dimer (31.1 mg, 0.1 mmol, 15 mol %) in DCM (15 mL) was stirred at room temperature under Argon. A solution of tert-butyl 2-diazoacetate (0.65 mL, 4.7 mmol, 1.0 eq.) in DCM (5 mL) was added dropwise. After the addition was complete the reaction mixture was stirred at room temperature for 16 hours. The mixture was concentrated under reduced pressure and the crude product purified by flash column chromatography (20% -100% ethyl acetate in hexane) to yield 374 mg (42%) of the desired product.

¹H NMR (400 MHz, CDCl₃): δ=3.96 (s, 2H), 3.81 (t, J=5.6 Hz, 2H), 3.68 (t, J=5.7 Hz, 2H), 1.83 (p, J=5.6 Hz, 2H), 1.48 (s, 9H). ¹³C NMR (101 MHz, CDCl₃): δ=170.4, 82.2, 69.7, 68.6, 60.8, 32.0, 28.2.

### Synthesis of tert-butyl 3-(3-iodopropoxy)propanoate (14)

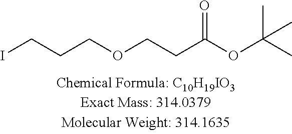

Compound 8 (3400 mg) was converted into the iodinated linker according to GP2 and yielded linker 14 in 99% (5160 mg).

¹H NMR (400 MHz, CDCl₃): δ=3.67 (t, J=6.4 Hz, 2H), 3.49 (t, J=5.8 Hz, 2H), 3.25 (t, J=6.8 Hz, 2H), 2.47 (t, J=6.4 Hz, 2H), 2.03 (tt, J=6.8, 5.8 Hz, 2H), 1.45 (s, 9H). ¹³C NMR (101 MHz, CDCl₃): δ=171.0, 80.7, 70.3, 66.7, 36.5, 33.5, 28.3, 3.5.

### Synthesis of tert-butyl 3-(2-iodoethoxy)propanoate (15)

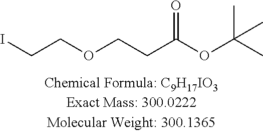

Compound 9 (597 mg) was converted into the iodinated linker according to GP2 and yielded linker 15 in 87% (812 mg).

¹H NMR (400 MHz, CDCl₃): δ=3.72 (q, J=6.5 Hz, 4H), 3.23 (t, J=6.9 Hz, 2H), 2.50 (t, J=6.4 Hz, 2H), 1.46 (s, 9H). ¹³C NMR (101 MHz, CDCl₃): δ=170.9, 80.9, 71.8, 66.6, 36.4, 28.3, 2.9.

### Synthesis of tert-butyl 3-(2-(2-iodoethoxy)ethoxy)propanoate (16)

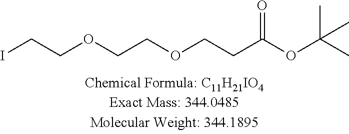 [SMILES: CC(C)(C)OC(=O)CCOCCOCCI]

Compound 10 (1000 mg) was converted into the iodinated linker according to GP2 and yielded linker 16 in 89% (1294 mg).

¹H NMR (400 MHz, CDCl₃): δ=3.78-3.68 (m, 4H), 3.65-3.58 (m, 4H), 3.25 (dd, J=7.4, 6.5 Hz, 2H), 2.50 (t, J=6.5 Hz, 2H), 1.44 (s, 9H). ¹³C NMR (101 MHz, CDCl₃): δ=171.0, 80.7, 72.1, 70.5, 70.3, 67.1, 36.4, 28.2, 3.0.

### Synthesis of tert-butyl 1-iodo-3,6,9,12-tetraoxapentadecan-15-oate (17)

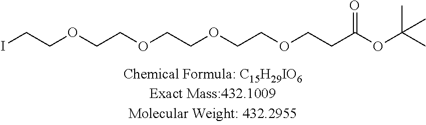 [SMILES: CC(C)(C)OC(=O)CCOCCOCCOCCOCCI]

Compound 11 (1500 mg) was converted into the iodinated linker according to GP2 and yielded linker 17 in 83% (1660 mg).

¹H NMR (400 MHz, CDCl₃): δ=3.78-3.59 (m, 16H), 3.30-3.21 (m, 2H), 2.50 (t, J=6.6 Hz, 2H), 1.44 (s, 9H). ¹³C NMR (101 MHz, CDCl₃): δ=171.05, 80.67, 72.12, 70.80, 70.74, 70.73, 70.65, 70.51, 70.35, 67.04, 36.40, 28.24, 3.04.

### Synthesis of tert-butyl 2-(2-iodoethoxy)acetate (18)

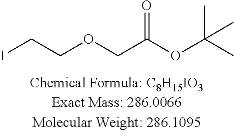

Compound 12 (552 mg) was converted into the iodinated linker according to GP2 and yielded linker 18 in 89% (790 mg).

¹H NMR (400 MHz, CDCl₃): δ=4.02 (d, J=1.1 Hz, 2H), 3.85-3.76 (m, 2H), 3.33-3.23 (m, 2H), 1.48 (d, J=1.1 Hz, 9H). ¹³C NMR (101 MHz, CDCl₃): δ=169.4, 82.1, 72.3, 68.8, 28.3, 2.2.

### Synthesis of tert-butyl 2-(3-iodopropoxy)acetate (19)

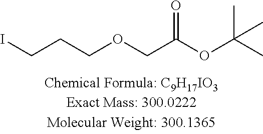

Compound 13 (375 mg) was converted into the iodinated linker according to GP2 and yielded linker 19 in 93% (548 mg).

¹H NMR (400 MHz, CDCl₃): δ=3.96 (s, 2H), 3.58 (t, J=5.8 Hz, 2H), 3.31 (t, J=6.8 Hz, 2H), 2.16-2.05 (m, 2H), 1.48 (s, 9H). ¹³C NMR (101 MHz, CDCl₃): δ=169.7, 81.8, 71.1, 69.1, 33.7, 28.3, 3.3.

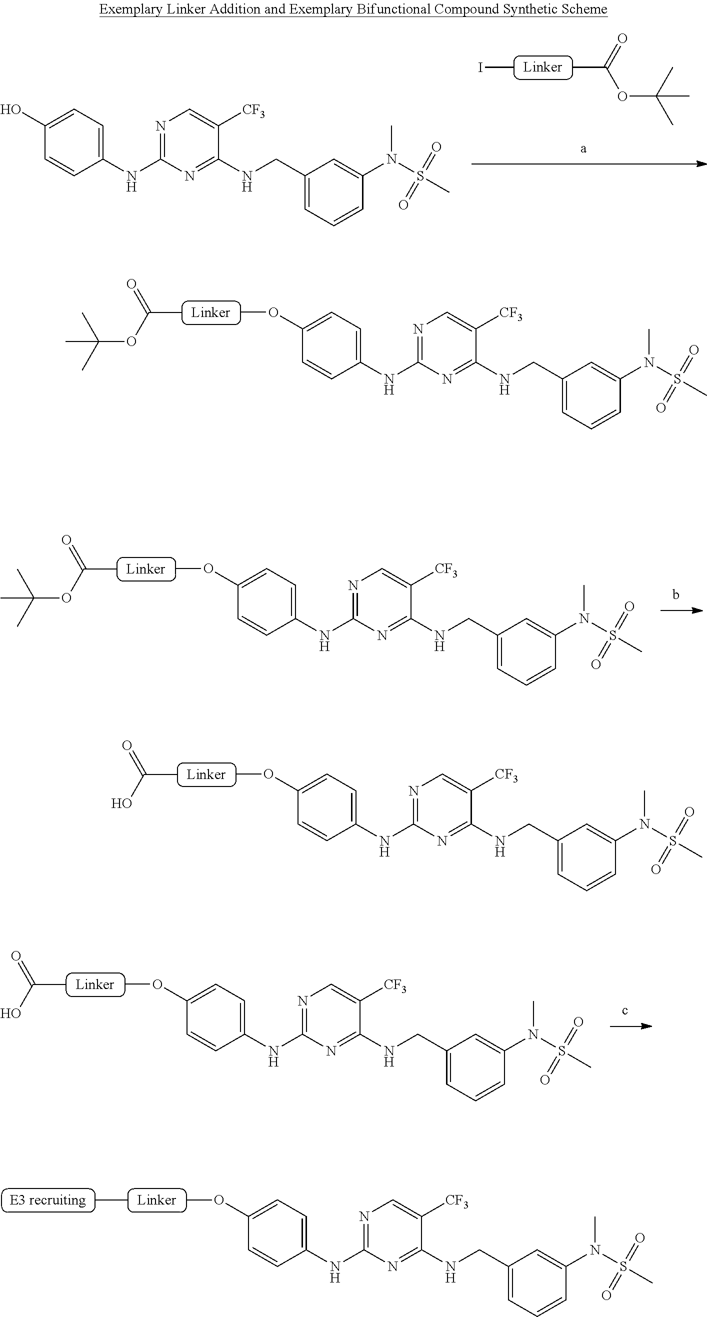

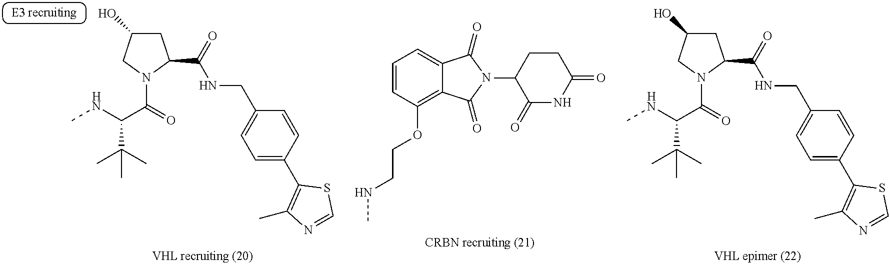

Reagents/Solvents/Conditions.

a) Linker 14-19, Cs₂CO₃, DMF, room temperature; b) 50% TFA in DCM, room temperature; c) HATU, DIEA, E3 recruiting element, DMF, room temperature.

VHL ligand 20 and epimer 22 were prepared according to previously reported procedures. Buckley, D. L.; Raina, K.; Darricarrere, N.; Hines, J.; Gustafson, J. L.; Smith, I. E.; Miah, A. H.; Harling, J. D.; Crews, C. M. HaloPROTACS: Use of Small Molecule PROTACs to Induce Degradation of HaloTag Fusion Proteins. ACS Chem. Biol. 2015, 10, 1831-1837.

CRBN ligand 21 was prepared according to previously reported procedures. Turk, B. E.; Jiang, H.; Liu, J. O. Binding of thalidomide to alpha1-acid glycoprotein may be involved in its inhibition of tumor necrosis factor alpha production. Proc. Natl. Acad. Sci. U.S.A. 1996, 93, 7552-7556.

### Synthesis of tert-butyl 3-(2-(4-((4-((3-(N-methylmethylsulfonamido)benzyl)amino)-5-(trifluoromethyl)pyrimidin-2-yl)amino)phenoxy)ethoxy)propanoate (23)

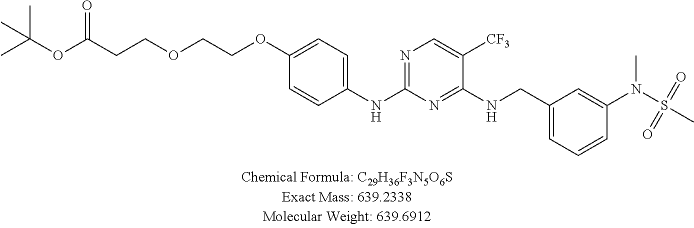 [SMILES: CN(c1cccc(CNc2nc(Nc3ccc(OCCOCCC(=O)OC(C)(C)C)cc3)ncc2C(F)(F)F)c1)S(C)(=O)=O]

7 (30 mg) was alkylated with iodinated linker 15 according to GP3 and yielded compound 23 in 73% (29.6 mg).

MS: calc. [M+H]⁺ for C₂₉H₃₇F₃N₅O₆S⁺=640.2411 [M+H]⁺; found=640.2523 [M+H]⁺

¹H NMR (400 MHz, CDCl₃): δ=8.15 (s, 1H), 7.37 (dt, J=7.6, 3.5 Hz, 4H), 7.27 (d, J=6.7 Hz, 1H), 7.21 (d, J=7.7 Hz, 1H), 6.88-6.79 (m, 2H), 4.69 (d, J=5.7 Hz, 2H), 4.08 (t, J=4.8 Hz, 2H), 3.79 (q, J=6.5, 5.8 Hz, 4H), 3.27 (s, 3H), 2.78 (s, 3H), 2.53 (t, J=6.5 Hz, 2H), 1.44 (s, 9H). ¹⁹F NMR (376 MHz, CDCl₃) δ=−61.25. ¹³C NMR (101 MHz, CDCl₃): δ=171.0, 158.9, 155.4, 141.9, 139.6, 131.8, 129.8, 126.3, 125.7, 124.7, 122.8, 122.4, 115.0, 80.8, 69.6, 67.8, 67.2, 44.7, 38.1, 36.4, 35.4, 28.2.

### Synthesis of tert-butyl 3-(3-(4-((4-((3-(N-methylmethylsulfonamido)benzyl)amino)-5-(trifluoromethyl)pyrimidin-2-yl)amino)phenoxy)propoxy)propanoate (24)

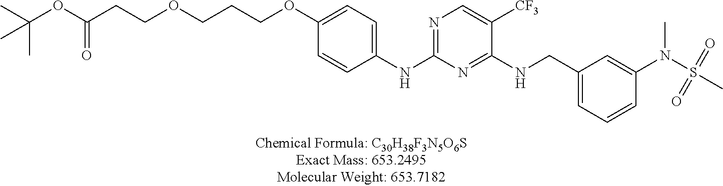 [SMILES: CN(c1cccc(CNc2nc(Nc3ccc(OCCCOCCC(=O)OC(C)(C)C)cc3)ncc2C(F)(F)F)c1)S(C)(=O)=O]

7 (69 mg) was alkylated with iodinated linker 14 according to GP3 and yielded compound 24 in 84% (81 mg).

MS: calc. [M+H]⁺ for C₃₀H₃₉F₃N₅O₆S⁺=654.2568 [M+H]⁺; found=654.2720 [M+H]⁺

¹H NMR (400 MHz, CDCl₃): δ=8.16 (s, 1H), 7.36 (d, J=8.1 Hz, 4H), 7.31-7.26 (m, 1H), 7.25-7.20 (m, 1H), 6.85-6.78 (m, 2H), 4.70 (d, J=5.7 Hz, 2H), 4.02 (t, J=6.3 Hz, 2H), 3.67 (t, J=6.5 Hz, 2H), 3.61 (t, J=6.1 Hz, 2H), 3.27 (s, 3H), 2.78 (s, 3H), 2.48 (t, J=6.4 Hz, 2H), 2.04-1.98 (m, 2H), 1.43 (s, 9H). ¹⁹F NMR (376 MHz, CDCl₃) δ=−61.19. ¹³C NMR (101 MHz, CDCl₃): δ=171.1, 158.9, 155.6, 154.3, 141.9, 139.7, 131.6, 129.8, 126.4, 125.8, 124.8, 124.0, 122.4, 115.2, 114.8, 110.2, 80.7, 67.6, 66.7, 65.3, 60.6, 44.7, 38.1, 36.5, 35.3, 29.8, 28.2.

### Synthesis of tert-butyl 3-(2-(2-(4-((4-((3-(N-methylmethylsulfonamido)benzyl)amino)-5-(trifluoromethyl)pyrimidin-2-yl)amino)phenoxy)ethoxy)ethoxy)propanoate (25)

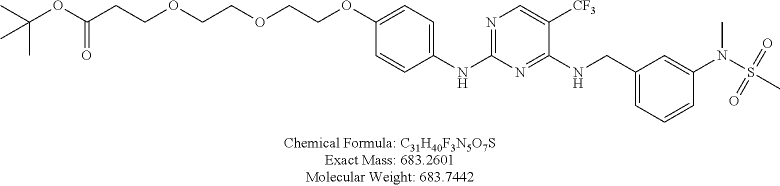 [SMILES: CN(c1cccc(CNc2nc(Nc3ccc(OCCOCCOCCC(=O)OC(C)(C)C)cc3)ncc2C(F)(F)F)c1)S(C)(=O)=O]

7 (15 mg) was alkylated with iodinated linker 16 according to GP3 and yielded compound 25 in 88% (19.1 mg).

MS: calc. [M+H]⁺ for C₃₁H₄₁F₃N₅O₇S⁺=684.2673 [M+H]⁺; found=684.2827 [M+H]⁺

¹H NMR (400 MHz, CDCl₃): δ=8.10 (s, 1H), 7.45-7.32 (m, 4H), 7.30 (d, J=8.0 Hz, 1H), 7.19 (d, J=7.8 Hz, 1H), 6.88-6.80 (m, 2H), 5.85 (s, 1H), 4.71 (d, J=5.7 Hz, 2H), 4.10 (t, J=4.9 Hz, 2H), 3.86-3.82 (m, 2H), 3.76-3.66 (m, 3H), 3.67-3.63 (m, 3H), 3.26 (s, 3H), 2.78 (s, 3H), 2.50 (t, J=6.5 Hz, 2H), 1.44 (s, 9H). ¹⁹F NMR (376 MHz, CDCl₃) δ=−61.11. ¹³C NMR (101 MHz, CDCl₃): δ=170.9, 158.6, 155.5, 141.8, 138.7, 131.2, 130.9, 130.0, 129.7, 128.8, 126.1, 125.6, 124.7, 122.5, 114.7, 80.5, 70.7, 70.4, 69.7, 67.7, 66.9, 44.8, 37.9, 36.2, 35.2, 28.1.

### Synthesis of tert-butyl 1-(4-((4-((3-(N-methylmethylsulfonamido)benzyl)amino)-5-(trifluoromethyl)pyrimidin-2-yl)amino)phenoxy)-3,6,9,12-tetraoxapentadecan-15-oate (26)

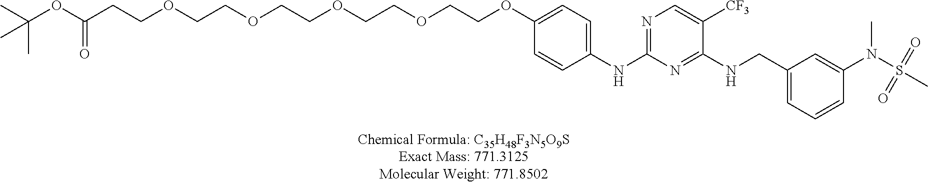 [SMILES: CN(c1cccc(CNc2nc(Nc3ccc(OCCOCCOCCOCCOCCC(=O)OC(C)(C)C)cc3)ncc2C(F)(F)F)c1)S(C)(=O)=O]

7 (15 mg) was alkylated with iodinated linker 17 according to GP3 and yielded compound 26 in 92% (22.7 mg).

MS: calc. [M+H]⁺ for C₃₅H₄₉F₃N₅O₉S⁺=772.3198 [M+H]⁺; found=772.3137 [M+H]⁺

¹H NMR (400 MHz, CDCl₃): δ=8.16 (s, 1H), 7.36 (dt, J=7.8, 3.5 Hz, 4H), 7.28-7.27 (m, 1H), 7.22 (d, J=7.7 Hz, 1H), 7.19 (s, 1H), 6.88-6.81 (m, 2H), 5.58 (d, J=6.1 Hz, 1H), 4.69 (d, J=5.7 Hz, 2H), 4.12-4.07 (m, 2H), 3.84 (dd, J=5.7, 4.1 Hz, 2H), 3.72-3.59 (m, 14H), 3.27 (s, 3H), 2.78 (s, 3H), 2.49 (t, J=6.6 Hz, 2H), 1.44 (s, 9H). ¹⁹F NMR (376 MHz, CDCl₃) δ=−61.16. ¹³C NMR (101 MHz, CDCl₃): δ=171.1, 161.2, 158.9, 155.2, 141.8, 139.9, 132.1, 129.8, 126.5, 126.3, 125.8, 124.7, 123.8, 122.2, 115.0, 80.7, 70.9, 70.8, 70.7, 70.6, 70.5, 69.9, 67.9, 67.0, 44.6, 38.1, 36.4, 35.3, 28.2.

### Synthesis of tert-butyl 2-(2-(4-((4-((3-(N-methylmethylsulfonamido)benzyl)amino)-5-(trifluoromethyl)pyrimidin-2-yl)amino)phenoxy)ethoxy)acetate (27)

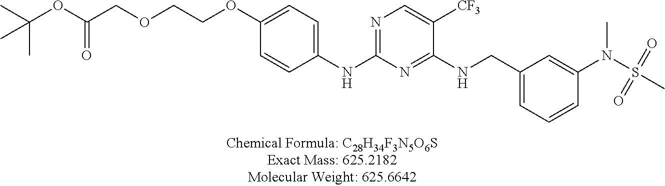 [SMILES: CN(c1cccc(CNc2nc(Nc3ccc(OCCOCC(=O)OC(C)(C)C)cc3)ncc2C(F)(F)F)c1)S(C)(=O)=O]

7 (30 mg) was alkylated with iodinated linker 18 according to GP3 and yielded compound 27 in 90% (36.0 mg).

MS: calc. [M+H]⁺ for C₂₈H₃₅F₃N₅O₆S⁺=626.2255 [M+H]⁺; found=626.2225 [M+H]⁺

¹H NMR (400 MHz, CDCl₃): δ=8.14 (s, 1H), 7.34 (p, J=4.4, 3.5 Hz, 4H), 7.24 (dd, J=5.5, 2.7 Hz, 1H), 7.20 (d, J=7.7 Hz, 1H), 6.83 (d, J=8.6 Hz, 2H), 4.66 (d, J=5.6 Hz, 2H), 4.12 (dd, J=5.7, 3.8 Hz, 2H), 4.06 (s, 2H), 3.91-3.85 (m, 2H), 3.24 (s, 3H), 2.76 (s, 3H), 1.46 (s, 9H). ¹⁹F NMR (376 MHz, CDCl₃) δ=−61.11. ¹³C NMR (101 MHz, CDCl₃): δ=169.7, 161.2, 158.9, 155.2, 155.1, 155.1, 141.8, 139.9, 132.3, 129.7, 126.3, 125.7, 124.7, 123.8, 122.3, 115.0, 81.8, 70.0, 69.3, 67.9, 44.6, 38.1, 35.3, 28.2.

### Synthesis of tert-butyl 3-(2-(4-((4-((3-(N-methylmethylsulfonamido)benzyl)amino)-5-(trifluoromethyl)pyrimidin-2-yl)amino)phenoxy)ethoxy)propanoate (28)

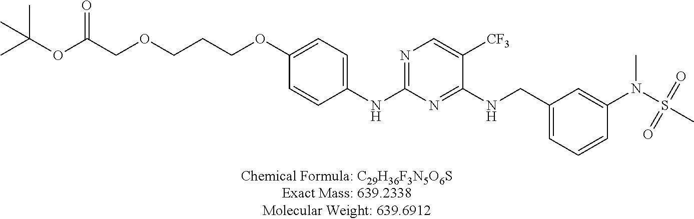 [SMILES: CN(c1cccc(CNc2nc(Nc3ccc(OCCCOCC(=O)OC(C)(C)C)cc3)ncc2C(F)(F)F)c1)S(C)(=O)=O]

7 (30 mg) was alkylated with iodinated linker 19 according to GP3 and yielded compound 28 in 80% (32.6 mg).

MS: calc. [M+H]⁺ for C₂₉H₃₇F₃N₅O₆S⁺=640.2411 [M+H]⁺; found=640.2501 [M+H]⁺

¹H NMR (400 MHz, CDCl₃): δ=8.16 (s, 1H), 7.37 (dd, J=8.6, 6.3 Hz, 4H), 7.30-7.26 (m, 1H), 7.22 (d, J=7.8 Hz, 1H), 6.87-6.79 (m, 2H), 4.69 (d, J=5.7 Hz, 2H), 4.06 (t, J=6.3 Hz, 2H), 3.96 (s, 2H), 3.69 (t, J=6.1 Hz, 2H), 3.26 (s, 3H), 2.77 (s, 3H), 2.08 (p, J=6.2 Hz, 2H), 1.47 (s, 9H). ¹⁹F NMR (376 MHz, CDCl₃) δ=−61.09. ¹³C NMR (101 MHz, CDCl₃): δ=169.8, 161.2, 158.9, 155.4, 155.2, 155.1, 141.8, 139.9, 131.9, 129.7, 126.3, 125.7, 124.8, 123.8, 122.3, 114.8, 81.7, 69.1, 68.3, 65.2, 44.6, 38.1, 35.3, 29.8, 28.2.

### Synthesis of (2S,4R)-1-((S)-3,3-dimethyl-2-(2-(2-(4-((4-((3-(N-methylmethylsulfonamido)benzyl)amino)-5-(trifluoromethyl)pyrimidin-2-yl)amino)phenoxy)ethoxy)acetamido)butanoyl)-4-hydroxy-N-(4-(4-methylthiazol-5-yl)benzyl)pyrrolidine-2-carboxamide (100)

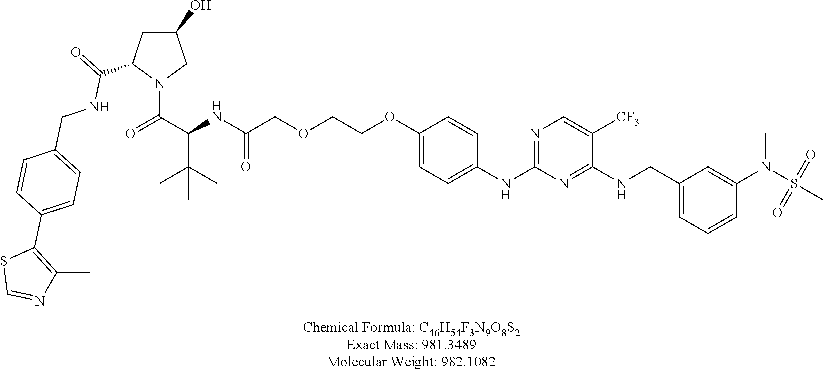 [SMILES: Cc1ncsc1-c1ccc(CNC(=O)[C@@H]2C[C@@H](O)CN2C(=O)[C@@H](NC(=O)COCCOc2ccc(Nc3ncc(C(F)(F)F)c(NCc4cccc(N(C)S(C)(=O)=O)c4)n3)cc2)C(C)(C)C)cc1]

The tert-butyl group of 27 (36.0 mg) was cleaved according to GP4. Afterwards, the obtained free acid was coupled with 20 according to GP5 to yield 100 in 27% (15.9 mg).

MS: calc. [M+H]⁺ for C₄₆H₅₅F₃N₉O₈S₂⁺=982.3562 [M+H]⁺; found=982.3773 [M+H]⁺, 491.6863 [M+2H]²⁺.

¹H NMR (400 MHz, CDCl₃): δ=8.58 (s, 1H), 8.04 (s, 1H), 7.27 (d, J=8.8 Hz, 6H), 7.18 (d, J=3.1 Hz, 2H), 7.16-7.10 (m, 2H), 6.82-6.73 (m, 2H), 4.64-4.57 (m, 3H), 4.51-4.41 (m, 3H), 4.25 (dd, J=15.0, 5.3 Hz, 1H), 4.09-4.01 (m, 2H), 4.00-3.93 (m, 3H), 3.82-3.75 (m, 2H), 3.56 (dd, J=11.2, 3.7 Hz, 1H), 3.18 (s, 3H), 2.70 (s, 3H), 2.45-2.41 (m, 1H), 2.40 (s, 3H), 2.08-1.97 (m, 1H), 0.88 (s, 9H). ¹⁹F NMR (376 MHz, CDCl₃) δ=−61.24. ¹³C NMR (101 MHz, CDCl₃): δ=171.27, 171.02, 170.29, 158.85, 155.04, 150.44, 148.57, 141.83, 139.65, 138.30, 132.22, 131.74, 131.00, 129.80, 129.67, 129.59, 128.22, 128.18, 126.25, 126.16, 126.07, 124.60, 123.48, 122.51, 115.17, 70.67, 70.59, 70.31, 67.82, 58.71, 57.21, 56.82, 44.70, 43.34, 38.11, 36.24, 35.36, 35.23, 26.54, 16.15.

### Synthesis of (2S,4R)-1-((S)-3,3-dimethyl-2-(2-(3-(4-((4-((3-(N-methylmethylsulfonamido)benzyl)amino)-5-(trifluoromethyl)pyrimidin-2-yl)amino)phenoxy)propoxy)acetamido)butanoyl)-4-hydroxy-N-(4-(4-methylthiazol-5-yl)benzyl)pyrrolidine-2-carboxamide (101)

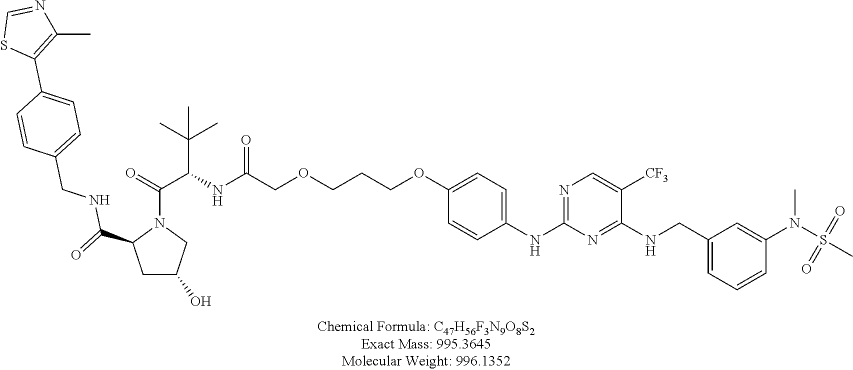 [SMILES: Cc1ncsc1-c1ccc(CNC(=O)[C@@H]2C[C@@H](O)CN2C(=O)[C@@H](NC(=O)COCCCOc2ccc(Nc3ncc(C(F)(F)F)c(NCc4cccc(N(C)S(C)(=O)=O)c4)n3)cc2)C(C)(C)C)cc1]

The tert-butyl group of 28 (29.8 mg) was cleaved according to GP4. Afterwards, the obtained free acid was coupled with 20 according to GP5 to yield 101 in 98% (49.6 mg).

MS: calc. [M+H]⁺ for C₄₇H₅₇F₃N₉O₈S₂⁺=996.3718 [M+H]⁺; found=996.3954 [M+H]⁺, 498.6934 [M+2H]²⁺.

¹H NMR (400 MHz, CDCl₃): δ=8.66 (s, 1H), 8.14 (s, 1H), 7.42-7.35 (m, 4H), 7.34-7.31 (m, 4H), 7.25-7.19 (m, 2H), 6.85-6.76 (m, 2H), 4.74-4.70 (m, 1H), 4.68 (d, J=5.7 Hz, 2H), 4.58-4.45 (m, 3H), 4.33 (dd, J=15.0, 5.3 Hz, 1H), 4.10-4.00 (m, 3H), 4.00-3.84 (m, 2H), 3.68 (td, J=6.3, 4.6 Hz, 2H), 3.62 (dd, J=11.3, 3.7 Hz, 1H), 3.26 (s, 3H), 2.77 (s, 3H), 2.57-2.51 (m, 1H), 2.49 (s, 3H), 2.15-2.09 (m, 1H), 2.09-2.02 (m, 2H), 0.93 (s, 9H). ¹⁹F NMR (376 MHz, CDCl₃) δ=−61.10. ¹³C NMR (101 MHz, CDCl₃): δ=171.44, 170.80, 170.26, 161.20, 158.90, 155.17, 150.44, 148.61, 141.81, 139.94, 138.21, 132.13, 131.71, 131.09, 129.77, 129.63, 128.26, 126.31, 126.05, 125.56, 124.62, 123.78, 122.31, 122.23, 114.88, 70.29, 70.25, 68.58, 64.88, 58.60, 57.06, 56.80, 44.62, 43.38, 38.12, 36.01, 35.35, 35.15, 29.67, 26.52, 16.19.

### Synthesis of (2S,4R)-1-((S)-3,3-dimethyl-2-(3-(2-(4-((4-((3-(N-methylmethylsulfonamido)benzyl)amino)-5-(trifluoromethyl)pyrimidin-2-yl)amino)phenoxy)ethoxy)propanamido)butanoyl)-4-hydroxy-7V-(4-(4-methylthiazol-5-yl)benzyl)pyrrolidine-2-carboxamide (102)

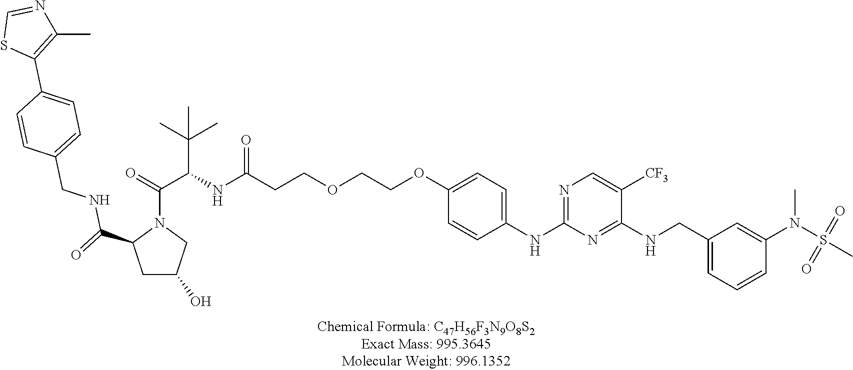 [SMILES: Cc1ncsc1-c1ccc(CNC(=O)[C@@H]2C[C@@H](O)CN2C(=O)[C@@H](NC(=O)CCOCCOc2ccc(Nc3ncc(C(F)(F)F)c(NCc4cccc(N(C)S(C)(=O)=O)c4)n3)cc2)C(C)(C)C)cc1]

The tert-butyl group of 23 (26.8 mg) was cleaved according to GP4. Afterwards, the obtained free acid was coupled with 20 according to GP5 to yield 102 in 77% (34.9 mg).

MS: calc. [M+H]⁺ for C₄₇H₅₇F₃N₉O₈S₂⁺=996.3718 [M+H]⁺; found=996.3858 [M+H]⁺, 498.6935 [M+2H]²⁺.

¹H NMR (400 MHz, CDCl₃): δ=8.59 (s, 1H), 8.08 (s, 1H), 7.32-7.24 (m, 8H), 7.20 (d, J=4.5 Hz, 1H), 7.15 (d, J=7.7 Hz, 1H), 6.79-6.72 (m, 2H), 4.64-4.59 (m, 3H), 4.51-4.39 (m, 3H), 4.24 (dd, J=15.0, 5.3 Hz, 1H), 4.05-3.96 (m, 3H), 3.75-3.72 (m, 2H), 3.70 (t, J=5.7 Hz, 2H), 3.53 (dd, J=11.4, 3.7 Hz, 1H), 3.19 (s, 3H), 2.71 (s, 3H), 2.41 (s, 3H), 2.40-2.36 (m, 2H), 2.09-1.99 (m, 2H), 0.86 (s, 9H). ¹⁹F NMR (376 MHz, CDCl₃) δ=−61.09. ¹³C NMR (101 MHz, CDCl₃): δ=172.09, 171.82, 171.03, 161.14, 158.87, 155.13, 155.08, 154.94, 150.43, 148.54, 141.77, 139.93, 138.28, 132.36, 131.72, 130.97, 129.74, 129.56, 128.17, 126.25, 125.97, 124.59, 123.75, 122.27, 115.00, 70.20, 69.91, 67.71, 67.41, 58.62, 57.89, 56.77, 44.57, 43.29, 38.11, 36.86, 36.16, 35.33, 35.00, 26.52, 16.15.

### Synthesis of (2S,4R)-1-((S)-3,3-dimethyl-2-(3-(3-(4-((4-((3-(N-methylmethylsulfonamido)benzyl)amino)-5-(trifluoromethyl)pyrimidin-2-yl)amino)phenoxy)propoxy)propanamido)butanoyl)-4-hydroxy-N-(4-(4-methylthiazol-5-yl)benzyl)pyrrolidine-2-carboxamide (103)

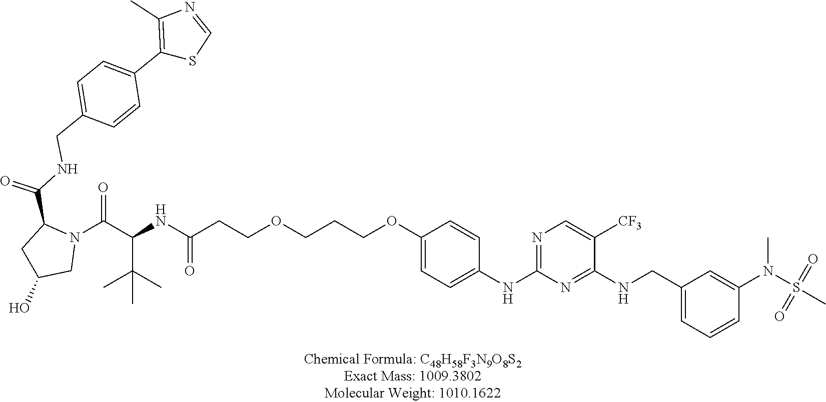 [SMILES: Cc1ncsc1-c1ccc(CNC(=O)[C@@H]2C[C@@H](O)CN2C(=O)[C@@H](NC(=O)CCOCCCOc2ccc(Nc3ncc(C(F)(F)F)c(NCc4cccc(N(C)S(C)(=O)=O)c4)n3)cc2)C(C)(C)C)cc1]

The tert-butyl group of 24 (74.1 mg) was cleaved according to GP4. Afterwards, the obtained free acid was coupled with 20 according to GP5 to yield 103 in 78% (97.8 mg).

MS: calc. [M+H]⁺ for C₄₈H₅₉F₃N₉O₈S₂⁺=1010.3875 [M+H]⁺; found=1010.3858 [M+H]⁺, 505.6914 [M+2H]²⁺.

¹H NMR (400 MHz, Methanol-D₄): δ=8.83 (d, J=1.6 Hz, 1H), 8.08-8.04 (m, 1H), 7.46-7.41 (m, 2H), 7.40-7.35 (m, 3H), 7.34-7.29 (m, 3H), 7.27-7.19 (m, 2H), 6.83-6.74 (m, 2H), 4.65 (d, J=9.3 Hz, 3H), 4.60-4.52 (m, 1H), 4.52-4.45 (m, 2H), 4.32 (dd, J=15.3, 4.1 Hz, 1H), 4.02 (t, J=6.3 Hz, 2H), 3.91-3.84 (m, 1H), 3.78 (dd, J=11.0, 3.9 Hz, 1H), 3.75-3.65 (m, 2H), 3.63 (td, J=6.2, 1.9 Hz, 2H), 3.18 (s, 3H), 2.75 (s, 3H), 2.57 (ddd, J=14.8, 7.6, 4.9 Hz, 1H), 2.50-2.45 (m, 1H), 2.44 (s, 3H), 2.24-2.17 (m, 1H), 2.08 (dt, J=8.5, 4.3 Hz, 1H), 2.05-1.97 (m, 2H), 1.02 (s, 9H). ¹⁹F NMR (376 MHz, Methanol-D₄) δ=−63.28. ¹³C NMR (101 MHz, Methanol-D₄): δ=174.43, 173.82, 172.11, 162.30, 159.95, 156.43, 155.36, 152.80, 148.97, 143.05, 142.16, 140.21, 133.68, 133.40, 131.46, 130.48, 130.33, 130.25, 129.41, 128.93, 127.09, 126.53, 125.82, 125.05, 123.54, 115.53, 71.07, 68.70, 68.01, 66.24, 60.81, 58.89, 58.01, 44.75, 43.69, 38.90, 38.54, 37.40, 36.85, 35.10, 30.84, 27.04, 15.84.

### Synthesis of (2S,4R)-1-((S)-3,3-dimethyl-2-(3-(2-(2-(4-((4-((3-(N-methylmethylsulfonamido)benzyl)amino)-5-(trifluoromethyl)pyrimidin-2-yl)amino)phenoxy)ethoxy)ethoxy)propanamido)butanoyl)-4-hydroxy-7V-(4-(4-methylthiazol-5-yl)benzyl)pyrrolidine-2-carboxamide (104)

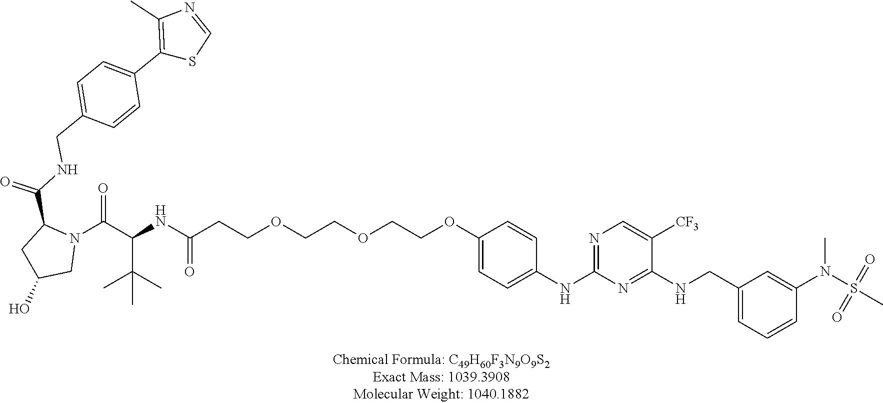 [SMILES: Cc1ncsc1-c1ccc(CNC(=O)[C@@H]2C[C@@H](O)CN2C(=O)[C@@H](NC(=O)CCOCCOCCOc2ccc(Nc3ncc(C(F)(F)F)c(NCc4cccc(N(C)S(C)(=O)=O)c4)n3)cc2)C(C)(C)C)cc1]

The tert-butyl group of 25 (20.0 mg) was cleaved according to GP4. Afterwards, the obtained free acid was coupled with 20 according to GP5 to yield 104 in 17% (5.5 mg).

MS: calc. [M+H]⁺ for C₄₉H₆₁F₃N₉O₉S₂⁺=1040.3980 [M+H]⁺; found=1040.4178 [M+H]⁺, 520.7077 [M+2H]²⁺.

¹H NMR (400 MHz, CDCl₃): δ=8.66 (s, 1H), 8.10 (s, 1H), 7.39-7.32 (m, 8H), 7.24 (s, 1H), 7.21 (d, J=7.6 Hz, 1H), 6.86 (d, J=8.9 Hz, 2H), 5.61 (s, 1H), 4.76-4.64 (m, 3H), 4.61-4.49 (m, 2H), 4.43 (d, J=8.2 Hz, 1H), 4.30 (dd, J=15.0, 5.2 Hz, 1H), 4.15-4.07 (m, 3H), 3.81 (dd, J=5.7, 3.8 Hz, 2H), 3.70-3.63 (m, 5H), 3.59 (dd, J=11.6, 3.5 Hz, 1H), 3.53-3.40 (m, 1H), 3.26 (s, 3H), 2.78 (s, 3H), 2.56-2.50 (m, 1H), 2.49 (s, 3H), 2.38 (d, J=6.7 Hz, 2H), 2.13 (dd, J=13.5, 8.2 Hz, 1H), 0.93 (s, 9H). ¹⁹F NMR (376 MHz, CDCl₃) δ=−61.15. ¹³C NMR (101 MHz, CDCl₃): δ=172.18, 171.87, 171.06, 171.04, 158.88, 155.22, 150.41, 148.60, 141.85, 139.76, 138.33, 132.12, 131.76, 131.03, 129.80, 129.59, 128.29, 126.29, 124.71, 122.53, 115.13, 70.66, 70.64, 70.34, 69.99, 67.93, 67.26, 58.57, 57.93, 56.78, 44.67, 43.38, 38.14, 36.82, 36.13, 35.36, 34.86, 26.55, 16.20.

### Synthesis of (2S,4R)-1-((S)-17-(tert-butyl)-1-(4-((4-((3-(N-methylmethylsulfonamido)benzyl)amino)-5-(trifluoromethyl)pyrimidin-2-yl)amino)phenoxy)-15-oxo-3,6,9,12-tetraoxa-16-azaoctadecan-18-oyl)-4-hydroxy-N-(4-(4-methylthiazol-5-yl)benzyl)pyrrolidine-2-carboxamide (105)

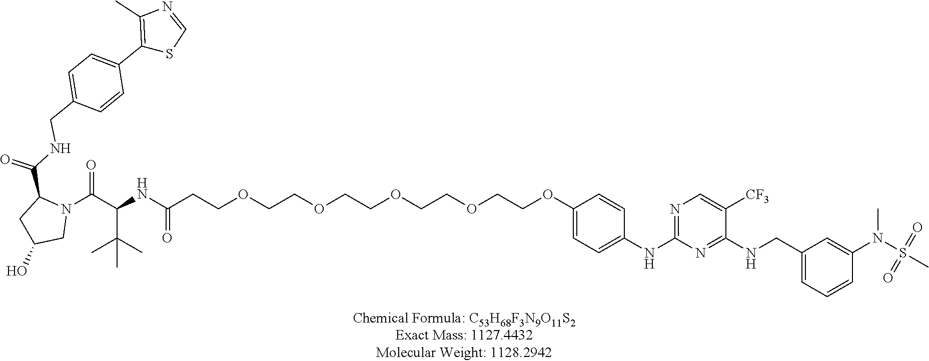 [SMILES: Cc1ncsc1-c1ccc(CNC(=O)[C@@H]2C[C@@H](O)CN2C(=O)[C@@H](NC(=O)CCOCCOCCOCCOCCOc2ccc(Nc3ncc(C(F)(F)F)c(NCc4cccc(N(C)S(C)(=O)=O)c4)n3)cc2)C(C)(C)C)cc1]

The tert-butyl group of 26 (13.7 mg) was cleaved according to GP4. Afterwards, the obtained free acid was coupled with 20 according to GP5 to yield 105 in 49% (10.4 mg).

MS: calc. [M+H]⁺ for C₅₃H₆₉F₃N₉O₁₁S₂⁺=1128.4505 [M+H]⁺; found=1128.4836 [M+H.

¹H NMR (400 MHz, CDCl₃): δ=8.67 (s, 1H), 8.11 (s, 1H), 7.40-7.32 (m, 8H), 7.26-7.24 (m, 1H), 7.21 (d, J=7.7 Hz, 1H), 6.84 (d, J=8.9 Hz, 2H), 5.60 (d, J=5.8 Hz, 1H), 4.74 (t, J=8.0 Hz, 1H), 4.67 (dd, J=5.8, 2.0 Hz, 2H), 4.56 (dd, J=15.0, 6.6 Hz, 1H), 4.50 (s, 1H), 4.43 (d, J=8.1 Hz, 1H), 4.31 (dd, J=15.0, 5.2 Hz, 1H), 4.14-4.06 (m, 3H), 3.83 (dd, J=5.6, 3.8 Hz, 2H), 3.70 (dd, J=6.1, 3.4 Hz, 2H), 3.66-3.61 (m, 10H), 3.57 (dd, J=6.0, 3.5 Hz, 3H), 3.25 (s, 3H), 2.77 (s, 3H), 2.49 (s, 3H), 2.47-2.43 (m, 1H), 2.39 (t, J=5.8 Hz, 2H), 2.17-2.10 (m, 1H), 0.94 (s, 9H). ¹⁹F NMR (376 MHz, CDCl₃) δ=−61.10. ¹³C NMR (101 MHz, CDCl₃): δ=172.29, 171.85, 171.22, 171.18, 161.02, 158.89, 155.10, 154.92, 150.43, 148.55, 141.84, 139.86, 138.35, 132.30, 131.78, 130.96, 129.77, 129.56, 128.24, 126.41, 126.29, 125.83, 124.73, 123.73, 122.45, 114.93, 70.90, 70.72, 70.66, 70.57, 70.55, 70.50, 70.28, 69.88, 67.92, 67.24, 58.59, 58.05, 56.86, 44.60, 43.32, 38.13, 36.66, 36.28, 35.34, 34.83, 26.56, 16.18.

### Synthesis of (2S,4S)-1-((S)-3,3-dimethyl-2-(3-(2-(4-((4-((3-(N-methylmethylsulfonamido)benzyl)amino)-5-(trifluoromethyl)pyrimidin-2-yl)amino)phenoxy)ethoxy)propanamido)butanoyl)-4-hydroxy-7V-(4-(4-methylthiazol-5-yl)benzyl)pyrrolidine-2-carboxamide (106)

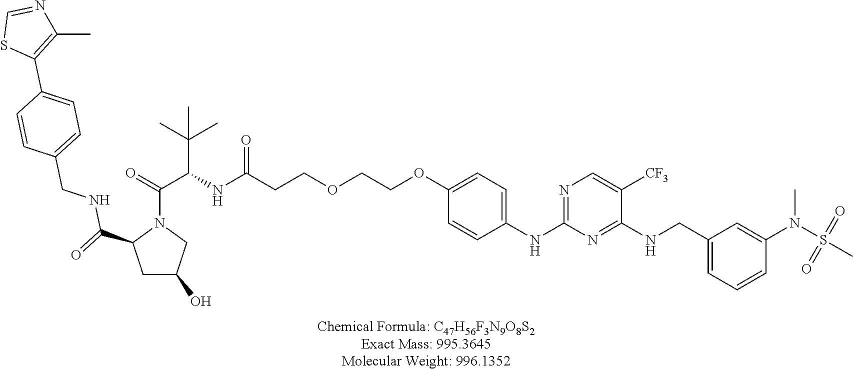 [SMILES: Cc1ncsc1-c1ccc(CNC(=O)[C@@H]2C[C@H](O)CN2C(=O)[C@@H](NC(=O)CCOCCOc2ccc(Nc3ncc(C(F)(F)F)c(NCc4cccc(N(C)S(C)(=O)=O)c4)n3)cc2)C(C)(C)C)cc1]

The tert-butyl group of 23 (8.0 mg) was cleaved according to GP4. Afterwards, the obtained free acid was coupled with 22 according to GP5 to yield 106 in 14% (1.9 mg).

MS: calc. [M+H]⁺ for C₄₇H₅₇F₃N₉O₈S₂⁺=996.3718 [M+H]⁺; found=996.3807 [M+H]⁺, 498.6856 [M+2H]²⁺.

¹H NMR (400 MHz, CDCl₃): δ=8.58 (s, 1H), 8.05 (s, 1H), 7.32-7.24 (m, 8H), 7.13 (d, J=4.5 Hz, 1H), 6.79-6.72 (m, 2H), 6.71 (d, J=7.7 Hz, 1H), 4.70 (s, 2H), 4.64-4.59 (m, 3H), 4.56-4.48 (m, 1H), 4.45-4.39 (m, 2H), 4.20 (dd, J=15.0, 5.3 Hz, 1H), 4.05-3.96 (m, 2H), 3.88-3.82 (m, 1H), 3.80-3.50 (m, 5H), 3.18 (s, 3H), 2.70 (s, 3H), 2.41 (s, 3H), 2.40-2.36 (m, 2H), 0.82 (s, 9H). ¹⁹F NMR (376 MHz, CDCl₃) δ=−61.18.

### Synthesis of N-(2-((2-(2,6-dioxopiperidin-3-yl)-1,3-dioxoisoindolin-4-yl)oxy)ethyl)-3-(2-(2-(4-((4-((3-(N-methylmethylsulfonamido)benzyl)amino)-5-(trifluoromethyl)pyrimidin-2-yl)amino)phenoxy)ethoxy)ethoxy)propanamide (107)

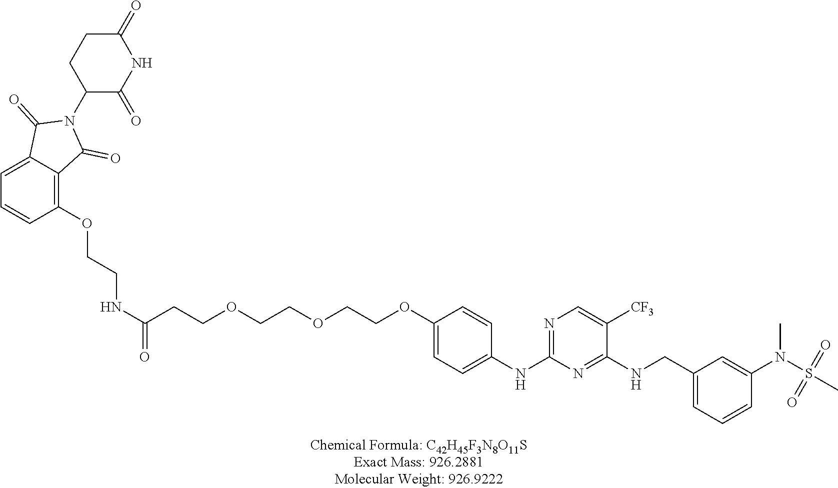 [SMILES: CN(c1cccc(CNc2nc(Nc3ccc(OCCOCCOCCC(=O)NCCOc4cccc5c4C(=O)N(C4CCC(=O)NC4=O)C5=O)cc3)ncc2C(F)(F)F)c1)S(C)(=O)=O]

The tert-butyl group of 25 (9.4 mg) was cleaved according to GP4. Afterwards, the obtained free acid was coupled with 21 according to GP5 to yield 108 in 52% (7.2 mg).

MS: calc. [M+H]⁺ for C₄₂H₄₆F₃N₈O₁₁S⁺=927.2953 [M+H]⁺; found=927.3005 [M+H]⁺, 464.1546 [M+2H]²⁺.

¹H NMR (400 MHz, CDCl₃): δ=8.19 (s, 1H), 7.66 (dd, J=8.4, 7.3 Hz, 1H), 7.46 (d, J=7.3 Hz, 1H), 7.39-7.33 (m, 4H), 7.30 (d, J=8.5 Hz, 1H), 7.27-7.26 (m, 1H), 7.24-7.19 (m, 1H), 7.14 (s, 1H), 6.81-6.73 (m, 2H), 5.61 (s, 1H), 4.93 (dd, J=12.0, 5.3 Hz, 1H), 4.70 (dd, J=5.7, 3.0 Hz, 2H), 4.27-4.14 (m, 2H), 4.10-4.04 (m, 2H), 3.81 (t, J=4.6 Hz, 2H), 3.77-3.61 (m, 8H), 3.56 (dd, J=13.6, 6.2 Hz, 1H), 3.25 (s, 3H), 2.89-2.80 (m, 1H), 2.77 (s, 3H), 2.74-2.66 (m, 1H), 2.50 (t, J=5.9 Hz, 2H), 2.14-2.07 (m, 1H). ¹⁹F NMR (376 MHz, CDCl₃) δ=−61.08. ¹³C NMR (101 MHz, CDCl₃): δ=172.32, 171.58, 167.07, 166.02, 160.83, 158.96, 156.31, 154.77, 141.84, 139.86, 136.83, 133.86, 132.51, 129.78, 126.26, 124.89, 121.98, 119.89, 117.56, 116.55, 114.79, 70.54, 70.28, 69.93, 68.04, 67.89, 67.14, 49.34, 44.67, 38.40, 38.14, 36.92, 35.34, 31.55, 22.93.

### Synthesis of N-(2-((2-(2,6-dioxopiperidin-3-yl)-1,3-dioxoisoindolin-4-yl)oxy)ethyl)-3-(3-(4-((4-((3-(N-methylmethylsulfonamido)benzyl)amino)-5-(trifluoromethyl)pyrimidin-2-yl)amino)phenoxy)propoxy)propanamide (108)

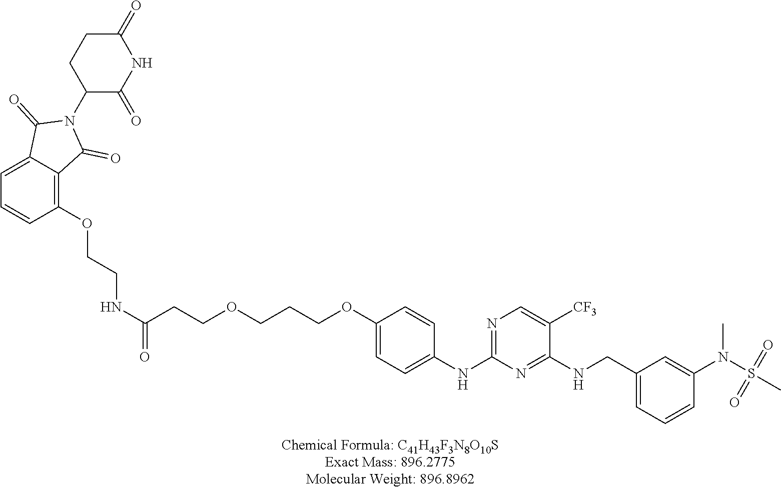 [SMILES: CN(c1cccc(CNc2nc(Nc3ccc(OCCCOCCC(=O)NCCOc4cccc5c4C(=O)N(C4CCC(=O)NC4=O)C5=O)cc3)ncc2C(F)(F)F)c1)S(C)(=O)=O]

The tert-butyl group of 24 (9.0 mg) was cleaved according to GP4. Afterwards, the obtained free acid was coupled with 21 according to GP5 to yield 107 in 42% (5.6 mg).

MS: calc. [M+H]⁺ for C₄₁H₄₄F₃N₈O₁₀S⁺=897.2848 [M+H]⁺; found=897.2910 [M+H]⁺, 449.1499 [M+2H]²⁺.

¹H NMR (400 MHz, CDCl₃): δ=8.18 (s, 1H), 7.67 (t, J=7.9 Hz, 1H), 7.46 (d, J=7.3 Hz, 1H), 7.39-7.32 (m, 4H), 7.27 (s, 1H), 7.22 (d, J=8.0 Hz, 2H), 6.86 (s, 1H), 6.76 (d, J=8.8 Hz, 2H), 5.61 (s, 1H), 4.93 (dd, J=12.0, 5.4 Hz, 1H), 4.69 (t, J=4.9 Hz, 2H), 4.25-4.19 (m, 1H), 4.17-4.10 (m, 1H), 3.99 (t, J=6.2 Hz, 2H), 3.70 (t, J=5.8 Hz, 3H), 3.63 (t, J=5.9 Hz, 2H), 3.57-3.46 (m, 1H), 3.24 (s, 3H), 2.89-2.78 (m, 2H), 2.76 (s, 3H), 2.74-2.65 (m, 1H), 2.48 (t, J=5.9 Hz, 2H), 2.14-2.08 (m, 1H), 2.01 (t, J=6.0 Hz, 2H). ¹⁹F NMR (376 MHz, CDCl₃) δ=−61.23. ¹³C NMR (101 MHz, CDCl₃): δ=172.17, 171.57, 167.04, 166.00, 158.94, 156.26, 155.11, 141.82, 139.86, 136.86, 133.89, 132.30, 129.79, 126.31, 122.06, 119.83, 117.66, 116.66, 114.68, 68.42, 67.60, 66.91, 65.29, 49.35, 44.67, 38.51, 38.13, 37.07, 35.30, 31.54, 29.48, 22.95.

### Synthesis of N-(2-((2-(2,6-dioxopiperidin-3-yl)-1,3-dioxoisoindolin-4-yl)oxy)ethyl)-1-(4-((4-((3-(N-methylmethylsulfonamido)benzyl)amino)-5-(trifluoromethyl)pyrimidin-2-yl)amino)phenoxy)-3,6,9,12-tetraoxapentadecan-15-amide (109)

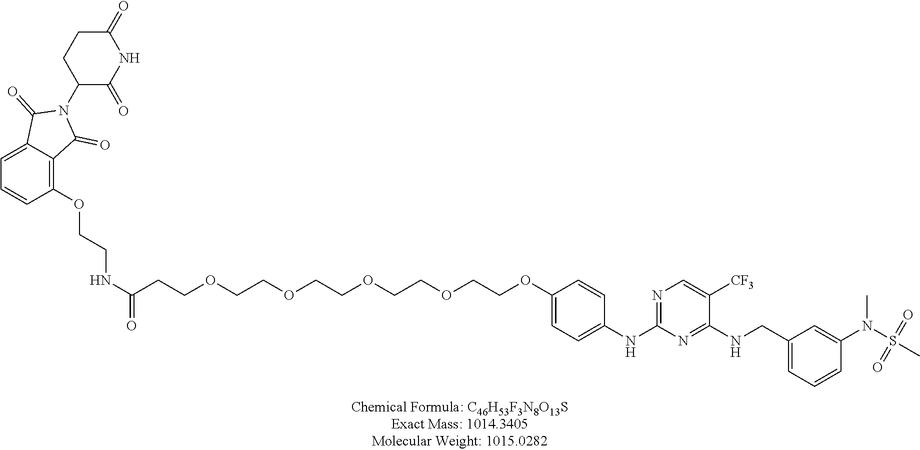 [SMILES: CN(c1cccc(CNc2nc(Nc3ccc(OCCOCCOCCOCCOCCC(=O)NCCOc4cccc5c4C(=O)N(C4CCC(=O)NC4=O)C5=O)cc3)ncc2C(F)(F)F)c1)S(C)(=O)=O]

The tert-butyl group of 26 (10.7 mg) was cleaved according to GP4. Afterwards, the obtained free acid was coupled with 21 according to GP5 to yield 109 in 57% (8.6 mg).

MS: calc. [M+H]⁺ for C₄₆H₅₄F₃N₈O₁₃S⁺=1015.3478 [M+H]⁺; found=1015.3580 [M+H, 508.1825 [M+2H]²⁺.

¹H NMR (400 MHz, CDCl₃): δ=8.20 (s, 1H), 7.68 (t, J=7.9 Hz, 1H), 7.48 (d, J=7.3 Hz, 1H), 7.36 (t, J=7.8 Hz, 4H), 7.28 (d, J=3.8 Hz, 2H), 7.22 (d, J=7.7 Hz, 1H), 7.11 (s, 1H), 6.85-6.78 (m, 2H), 5.62 (s, 1H), 4.92 (dd, J=12.1, 5.3 Hz, 1H), 4.69 (d, J=5.7 Hz, 2H), 4.24 (h, J=4.8 Hz, 2H), 4.09 (dd, J=5.8, 3.7 Hz, 2H), 3.81 (dd, J=5.7, 3.8 Hz, 2H), 3.73-3.66 (m, 5H), 3.66-3.62 (m, 3H), 3.59 (d, J=7.7 Hz, 8H), 3.25 (s, 3H), 2.90-2.79 (m, 2H), 2.77 (s, 3H), 2.75-2.64 (m, 1H), 2.48 (t, J=6.1 Hz, 2H), 2.11 (ddd, J=10.3, 4.7, 2.5 Hz, 1H). ¹⁹F NMR (376 MHz, CDCl₃) δ=−61.10. ¹³C NMR (101 MHz, CDCl₃): δ=171.93, 171.54, 168.93, 166.88, 165.85, 158.78, 156.21, 154.87, 141.69, 139.67, 136.67, 133.68, 132.10, 129.60, 126.13, 124.72, 121.95, 119.84, 117.60, 116.51, 114.86, 70.76, 70.54, 70.50, 70.37, 70.27, 70.19, 69.74, 68.54, 67.78, 67.07, 49.13, 44.48, 38.39, 37.95, 36.85, 35.16, 31.39, 22.66.

### Experimental Section: Biology

Enzymatic inhibition has proven to be a successful modality for the development of many small molecule drugs. In recent years, small molecule-induced protein degradation has emerged as an orthogonal therapeutic strategy that has the potential to expand the druggable target space. Fak is a key player in tumor invasion and metastasis, acting simultaneously as a kinase and a scaffold for several signaling proteins. While previous efforts to modulate Fak activity were limited to kinase inhibitors with low success in clinical studies, protein degradation offers a possibility to simultaneously block Fak's kinase signaling and scaffolding capabilities. The data below relates to the development of selective and potent Fak degrading exemplary compounds, which outperforms clinical candidate defactinib with respect to Fak activation as well as Fak-mediated cell migration and invasion. These results underline the potential that the PROTACs offer in expanding the druggable space and controlling protein functions that are not easily addressed by traditional small molecule therapeutics.

Introduction.

Fak is a cytoplasmic tyrosine kinase that controls many aspects of tumor growth (e.g., invasion, metastasis and angiogenesis) through kinase-dependent and kinase-independent mechanisms. In addition to its central kinase domain, Fak is comprised of three additional domains, a N-terminal four-point-one, ezrin, radixin, moesin (PERM) domain, a proline-rich region and a focal adhesion targeting (FAT)C-terminal domain, all of which mediate Fak kinase-independent signaling. Through its scaffolding domains Fak is involved in the formation of large signaling complexes primarily at the plasma membrane. Fak activation can be triggered upon engaging membrane proteins such as integrins resulting in Fak PERM domain displacement and subsequent autophosphorylation at Y397. Phosphorylation at Y397 creates a binding site for Src-family kinases, which phosphorylate the kinase domain activation loop (Y576 and Y577) leading to full Fak activation and formation of an activated Fak-Src complex. Increased Fak expression and activity can be found in primary and metastatic cancers of many tissues and is often associated with poor overall patient survival. This has rendered Fak an interesting target for drug discovery with multiple compounds in clinical trials. However, the current medicinal chemistry toolbox limits the development of chemical entities to Fak kinase inhibitors, thus ignoring the Fak scaffolding role. While some of these compounds have proven effective in preclinical studies, clinical success has yet to be observed. Thus far, the leading Fak inhibitor defactinib (Verastem VS-6063), failed its initial clinical trial targeting malignant pleural mesothelioma stem cells, although it is further being evaluated in combination with the anti-PD-1 immune checkpoint antibody, avelumab, for advanced ovarian cancer. Nevertheless, many essential functions mediated by the Fak scaffolding role are still beyond the reach of any kinase inhibitor. To overcome the mechanistic shortcomings of Fak kinase inhibitors, highly selective low nanomolar Fak degraders were designed. Exemplary compound 102 significantly exceeded the effects of defactinib on Fak signaling, as well as on cell migration and invasion, in human triple negative breast cancer (TNBC) cells.

Due to the mode of action (MOA) based limitations of Fak kinase inhibitors, the present disclosure developed bifunctional molecules that degrade Fak using the cells' own degradation machinery to address Fak kinase-independent functions. The bifunctional molecules combined a E3 ligase recruiting element with a protein of interest (POI) targeting warhead to facilitate subsequent POI ubiquitination and degradation by the ubiquitin proteasome system.

Cell Lines.

PC3 cells were cultured in F12-K (Kaighn's Modification of Ham's F-12 Medium), supplemented with 10% fetal bovine serum (FBS) and 1% penicillin-streptomycin at 37° C. and 5% CO₂. MDA-MB-231 cells were cultured in RPMI-1640 (ATCC), supplemented with 10% FBS and 1% penicillin-streptomycin at 37° C. and 5% CO₂.

Immunoblotting.

If not indicated otherwise, cells were seeded and grown to 80% confluency and were treated with compound or control for 24 hours. Subsequently, the growth media was removed and the cells lysed by the addition of lysis buffer (25 mM Tris, pH 7.4; 1% NP-40, 0.25% deoxycholate, 1 mM sodium vanadate, 10 mM sodium fluoride, 10 mM sodium pyrophosphate, 20 mM β-glycerophosphate and 1× complete EDTA-free protease inhibitor cocktail (Roche)). After 20 minutes the mixture was spun down at 16,000×g for 10 minutes at 4° C. to pellet insoluble materials. Protein concentration of supernatants were determined via BCA assay (Thermo Fisher) before addition of NuPAGE sample buffer containing 5% β-Me and boiling at 95° C. for 10 minutes. Equal amounts of protein were subjected to SDS-PAGE and subsequent electrophoretic transfer onto nitrocellulose membrane. Rabbit antibodies were purchased from Cell Signaling: Fak (3285), p-Fak (3283), p-Paxillin (2541), p-Akt (S473)(4060), GAPDH (2118), Androgen Receptor (5153), p-Src(Y527)(2105), p-S6RP (2215). Mouse antibodies were purchased from Cell Signaling: tubulin (3873), S6RP (2317). Secondary antibody ∝-rabbit (31460) or ∝-mouse (31444) was coupled to horseradish peroxidase and purchased from Thermo Fisher. Immunoblots were developed using enhanced chemiluminescence and visualized using a Bio-Rad Chemi-Doc MP Imaging System and quantitated with Image Lab v.5.2.1 software (Bio-Rad Laboratories). Data analysis and statistics was performed using Prism 7.0 (GraphPad).

Cell Proliferation Assays.

Cells were seeded in 96-well plates and treated with exemplary compound or control as indicated. At desired time points culture medium was supplemented with 330 mg/mL MTS (Promega) and 25 mM phenazine methosulfate (Sigma) and incubated at 37° C. Mitochondrial reduction of MTS to the formazan derivative was monitored by measuring the medium's absorbance at 490 nm using a Wallac Victor 2 plate reader (Perkin-Elmer Life Sciences). Data analysis and statistics was performed using Prism 7.0 (GraphPad).

KinomeScan.

The Kinase engagement assay was performed by DiscoverX assessing binding abilities towards a set of 468 kinases. Exemplary compound 102 and defactinib were screened at a concentration of 1000 nM.

Kinase Activity Assay.

Kinase activity assays were performed by Reaction Biology Corp. Compounds were tested in 10-dose IC₅₀ duplicate mode with a 3-fold serial dilution starting at 1 μM. Control compound, staurosporine, was tested in 10-dose IC₅₀ mode with 4-fold serial dilution starting at 20 μM. Reactions were carried out at 10 μM ATP. IC₅₀ values were calculated using Prism 7.0 (GraphPad).

RPPA (Reverse Phase Protein Array).

RPPA analysis was performed by MD Anderson Cancer Center RPPA core facility. MDA-MB-231 cells were grown in the presence of 10% FBS, trypsinized and allowed to reattach for 8 hours in the presence of DMSO (0.1%), exemplary compound 102 (500 nM) or defactinib before cells were subjected to lysis and samples prepared according to protocols provided by MD Anderson.

Wound Healing Assay.

MDA-MB-231 cells were maintained in complete growth medium (RPMI-1640) at 37° C. supplied with 5% CO₂. Cells (1×10⁶) were split in to a 12-well plate. After 24 hours an even wound was created across each well using a sterile 10 μL pipette tip and the cells were washed with warm phosphate-buffered solution (PBS) twice to remove any floating or dead cells. This time point was considered as 0 hours and cells were incubated in fresh medium containing exemplary compound or control as indicated, for 24 hours. Images of wounded area were captured at 0 hour and after 24 hours using a camera attached to a light microscope. Images were analyzed by ImageJ software and wounded area was quantified. The area of the remaining wound at 24 hours was subtracted from the area of the wound at 0 hour. Percent wound healing (migration) was calculated and data presented as a bar graph using Prism 7.0 (GraphPad). Differences between groups were analyzed by Welch's t-test and considered significant when p<0.05.

Transwell Invasion Assay.

On the first day, 0.2× Basement Membrane Extract (BME) working solution was prepared by diluting 5×BME stock solution in 1× Travigen Inc. coating buffer. Briefly, 100 μL of 10× coating buffer was diluted in 900 μL of sterile water to make 1× coating buffer. Then 960 μL of 1× coating buffer was mixed with 40 μL of 5×BME to make working 0.2×BME solution. Corning Transwell permeable inserts (Costar Transwell chambers, Corning) were placed on a 24-well plate and 100 μL of 0.2×BME solution was added to each Transwell insert and incubated for 16 hours. Following day, MDA-MB-231 cells were trypsinized and cells were suspended in serum free medium. Approximately 100 μL from cell suspension (˜3×10⁵ cells) was added to each Transwell insert followed by another 100 μL of exemplary compound or control containing serum free RPMI medium. The lower chamber was filled with 10% FBS containing RPMI medium and the whole setup was incubated at 37° C. 1-5% CO₂ for 24 hours. After 24 hours, cell culture medium was removed from both lower and upper chambers and Transwell inserts were washed three times with PBS. Non-invasive cells were removed using a cotton swab and bottom side of the membrane of Transwell inserts were fixed with 4% formaldehyde for 10 minutes at room temperature followed by permeabilization with PBST (pH-7.4, 50 mM Tris-HCl, 150 mM NaCl, 0.1% Triton-X100) for another 10 minutes. Inserts were washed once with PBS and stained with 0.2% (W/V) crystal violet solution for 20 minutes at room temperature. Inserts were then extensively washed with PBS and once with water to remove all excess dye and salts. Cells migrated through the membrane were captured using a camera attached to a light microscope. Images were then analyzed by ImageJ software and number of cells on the bottom side of the membrane were counted and presented as a bar graph using Prism 7.0 (GraphPad). Differences among groups were analyzed by Welch's t-test and considered significant when P<0.05.

Examination of Target Protein (Fak) Degradation.

The following exemplary compounds demonstrated target protein degradation when tested under the conditions described above:

TABLE 1
Exemplary compounds of the present disclosure:

| Ex. | | |
|---|---|---|
| No. | Chemical Structure | Compound Name |
| 100 | 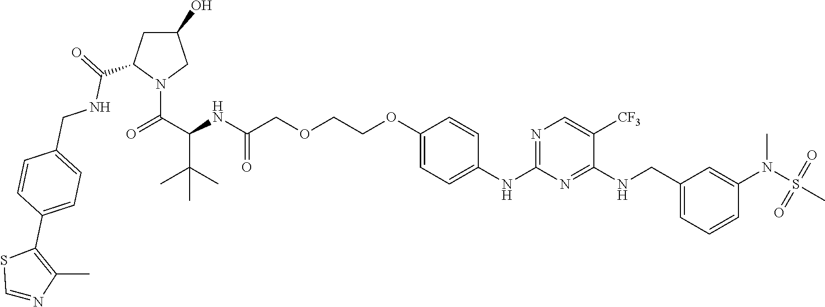 [SMILES: Cc1ncsc1-c1ccc(CNC(=O)[C@@H]2C[C@@H](O)CN2C(=O)[C@@H](NC(=O)COCCOc2ccc(Nc3ncc(C(F)(F)F)c(NCc4cccc(N(C)S(C)(=O)=O)c4)n3)cc2)C(C)(C)C)cc1] | (2S,4R)-1-((S)-3,3-dimethyl-2-(2-(2- (4-((4-((3-(N- methylmethylsulfonamido)benzyl) amino)-5-(trifluoromethyl)pyrimidin-2- yl)amino)phenoxy)ethoxy)acetamido) butanoyl)-4-hydroxy-N-(4-(4- methylthiazol-5-yl)benzyl)pyrrolidine- 2-carboxamide |
| 101 | 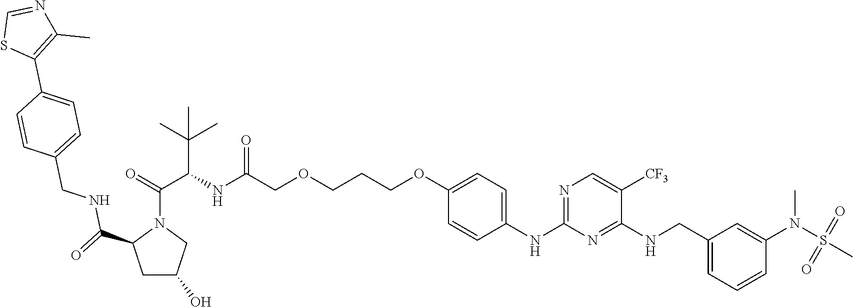 [SMILES: Cc1ncsc1-c1ccc(CNC(=O)[C@@H]2C[C@@H](O)CN2C(=O)[C@@H](NC(=O)COCCCOc2ccc(Nc3ncc(C(F)(F)F)c(NCc4cccc(N(C)S(C)(=O)=O)c4)n3)cc2)C(C)(C)C)cc1] | (2S,4R)-1-((S)-3,3-dimethyl-2-(2-(3- (4-((4-((3-(N- methylmethylsulfonamido)benzyl) amino)-5-(trifluoromethyl)pyrimidin-2- yl)amino)phenoxy)propoxy)acetamido) butanoyl)-4-hydroxy-N-(4-(4- methylthiazol-5-yl)benzyl)pyrrolidine- 2-carboxamide |
| 102 | 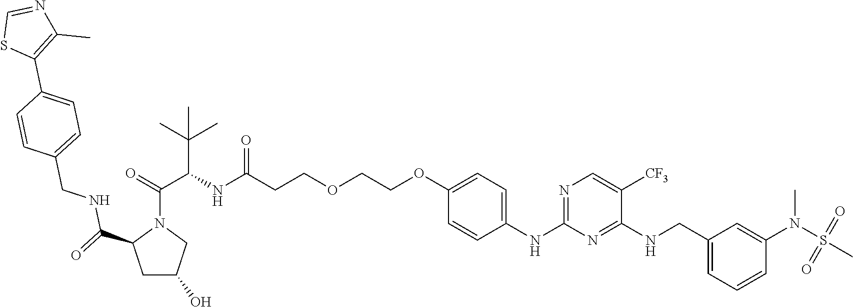 [SMILES: Cc1ncsc1-c1ccc(CNC(=O)[C@@H]2C[C@@H](O)CN2C(=O)[C@@H](NC(=O)CCOCCOc2ccc(Nc3ncc(C(F)(F)F)c(NCc4cccc(N(C)S(C)(=O)=O)c4)n3)cc2)C(C)(C)C)cc1] | (2S,4R)-1-((S)-3,3-dimethyl-2-(3-(2- (4-((4-((3-(N- methylmethylsulfonamido)benzyl) amino)-5-(trifluoromethyl)pyrimidin-2- yl)amino)phenoxy)ethoxy)propanamido) butanoyl)-4-hydroxy-N-(4-(4- methylthiazol-5-yl)benzyl)pyrrolidine- 2-carboxamide |
| 103 | 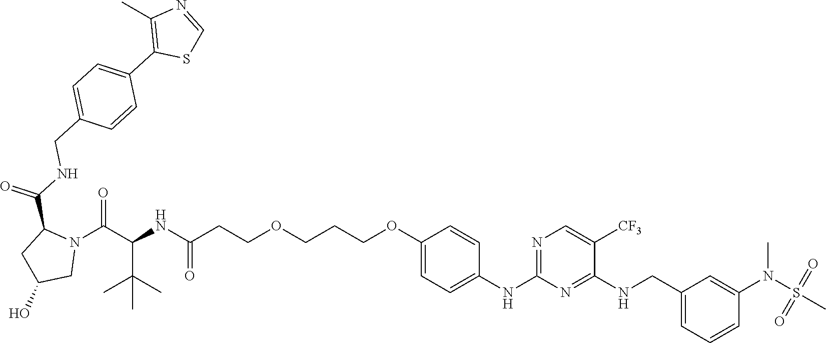 [SMILES: Cc1ncsc1-c1ccc(CNC(=O)[C@@H]2C[C@@H](O)CN2C(=O)[C@@H](NC(=O)CCOCCCOc2ccc(Nc3ncc(C(F)(F)F)c(NCc4cccc(N(C)S(C)(=O)=O)c4)n3)cc2)C(C)(C)C)cc1] | (2S,4R)-1-((S)-3,3-dimethyl-2-(3-(3- (4-((4-((3-(N- methylmethylsulfonamido)benzyl) amino)-5-(trifluoromethyl)pyrimidin-2- yl)amino)phenoxy)propoxy)propanamido) butanoyl)-4-hydroxy-N-(4-(4- methylthiazol-5-yl)benzyl)pyrrolidine- 2-carboxamide |
| 104 | 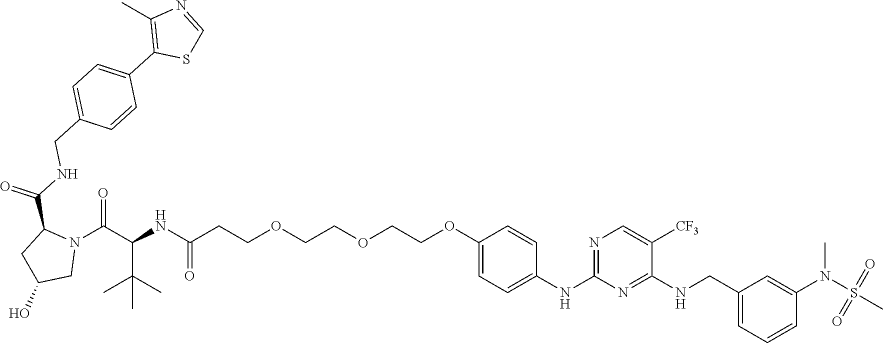 [SMILES: Cc1ncsc1-c1ccc(CNC(=O)[C@@H]2C[C@@H](O)CN2C(=O)[C@@H](NC(=O)CCOCCOCCOc2ccc(Nc3ncc(C(F)(F)F)c(NCc4cccc(N(C)S(C)(=O)=O)c4)n3)cc2)C(C)(C)C)cc1] | (2S,4R)-1-((S)-3,3-dimethyl-2-(3-(2- (2-(4-((4-((3-(N- methylmethylsulfonamido)benzyl) amino)-5-(trifluoromethyl)pyrimidin-2- yl)amino)phenoxy)ethoxy)ethoxy)propan- amido)butanoyl)-4-hydroxy-N-(4-(4- methylthiazol-5-yl)benzyl)pyrrolidine- 2-carboxamide |
| 105 | 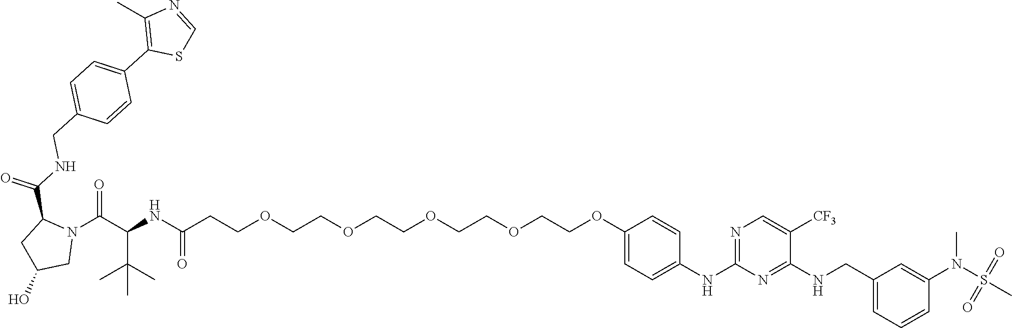 [SMILES: Cc1ncsc1-c1ccc(CNC(=O)[C@@H]2C[C@@H](O)CN2C(=O)[C@@H](NC(=O)CCOCCOCCOCCOCCOc2ccc(Nc3ncc(C(F)(F)F)c(NCc4cccc(N(C)S(C)(=O)=O)c4)n3)cc2)C(C)(C)C)cc1] | (2S,4R)-1-((S)-17-(tert-butyl)-1-(4-((4- ((3-(N- methylmethylsulfonamido)benzyl) amino)-5-(trifluoromethyl)pyrimidin-2- yl)amino)phenoxy)-15-oxo-3,6,9,12- tetraoxa-16-azaoctadecan-18-oyl)-4- hydroxy-N-(4-(4-methylthiazol-5- yl)benzyl)pyrrolidine-2-carboxamide |
| 106 | 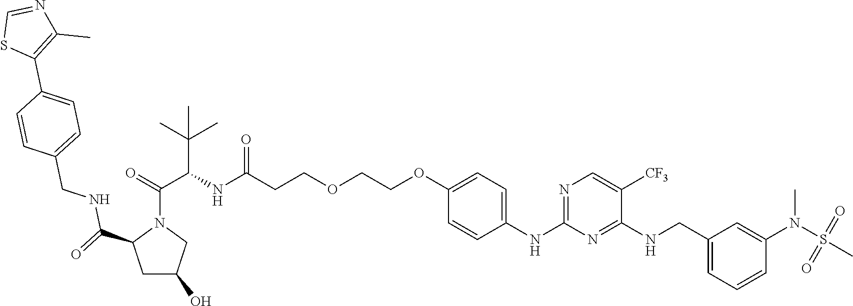 [SMILES: Cc1ncsc1-c1ccc(CNC(=O)[C@@H]2C[C@H](O)CN2C(=O)[C@@H](NC(=O)CCOCCOc2ccc(Nc3ncc(C(F)(F)F)c(NCc4cccc(N(C)S(C)(=O)=O)c4)n3)cc2)C(C)(C)C)cc1] | (2S,4S)-1-((S)-3,3-dimethyl-2-(3-(2-(4- ((4-((3-(N- methylmethylsulfonamido)benzyl) amino)-5-(trifluoromethyl)pyrimidin-2- yl)amino)phenoxy)ethoxy)propanamido) butanoyl)-4-hydroxy-N-(4-(4- methylthiazol-5-yl)benzyl)pyrrolidine- 2-carboxamide |
| 107 | 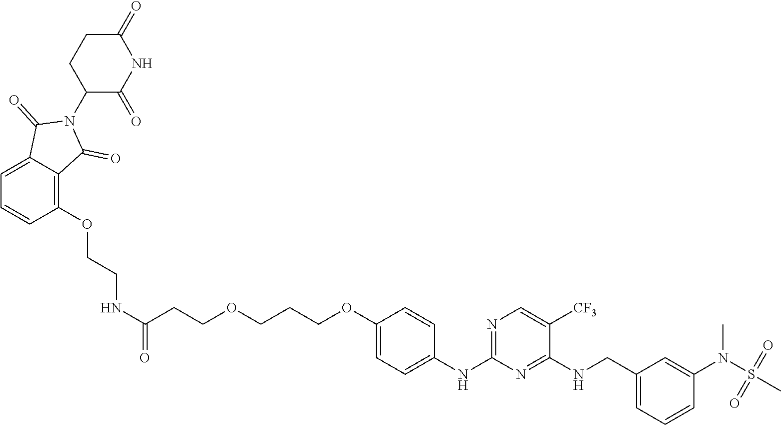 [SMILES: CN(c1cccc(CNc2nc(Nc3ccc(OCCCOCCC(=O)NCCOc4cccc5c4C(=O)N(C4CCC(=O)NC4=O)C5=O)cc3)ncc2C(F)(F)F)c1)S(C)(=O)=O] | N-(2-((2-(2,6-dioxopiperidin-3-yl)-1,3- dioxoisoindolin-4-yl)oxy)ethyl)-3-(2- (2-(4-((4-((3-(N- methylmethylsulfonamido)benzyl) amino)-5-(trifluoromethyl)pyrimidin-2- yl)amino)phenoxy)ethoxy)ethoxy)propan- amide |
| 108 | 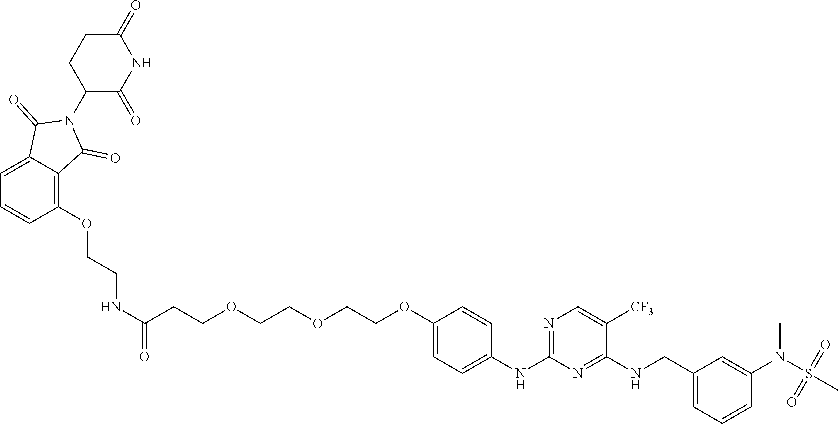 [SMILES: CN(c1cccc(CNc2nc(Nc3ccc(OCCOCCOCCC(=O)NCCOc4cccc5c4C(=O)N(C4CCC(=O)NC4=O)C5=O)cc3)ncc2C(F)(F)F)c1)S(C)(=O)=O] | N-(2-((2-(2,6-dioxopiperidin-3-yl)-1,3- dioxoisoindolin-4-yl)oxy)ethyl)-3-(3- (4-((4-((3-(N- methylmethylsulfonamido)benzyl) amino)-5-(trifluoromethyl)pyrimidin-2- yl)amino)phenoxy)propoxy)propanamide |
| 109 | 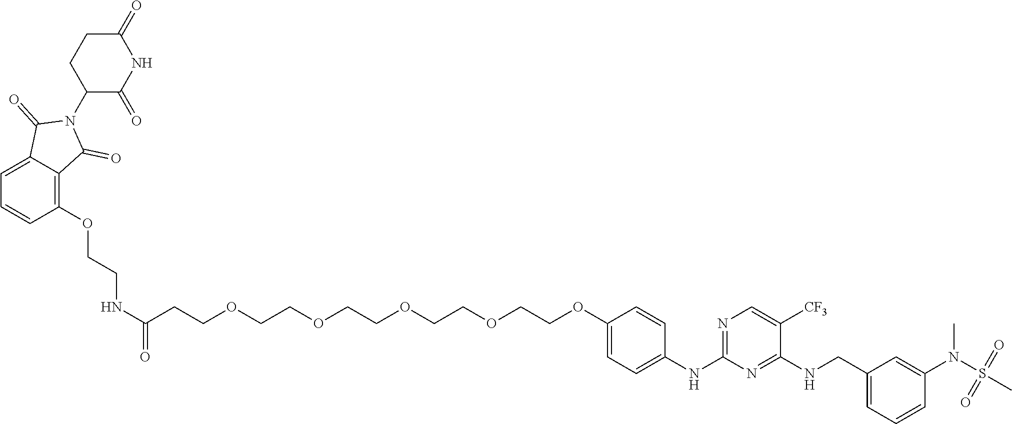 [SMILES: CN(c1cccc(CNc2nc(Nc3ccc(OCCOCCOCCOCCOCCC(=O)NCCOc4cccc5c4C(=O)N(C4CCC(=O)NC4=O)C5=O)cc3)ncc2C(F)(F)F)c1)S(C)(=O)=O] | N-(2-((2-(2,6-dioxopiperidin-3-yl)-1,3- dioxoisoindolin-4-yl)oxy)ethyl)-1-(4- ((4-((3-(N- methylmethylsulfonamido)benzyl) amino)-5-(trifluoromethyl)pyrimidin-2- yl)amino)phenoxy)-3,6,9,12- tetraoxapentadecan-15-amide |

TABLE 2
Target protein degradation via Exemplary Compounds

| Ex No. or | | Liner | IC₅₀ (nM) | DC₅₀ | D_{Max} (%) | |
|---|---|---|---|---|---|---|
| Compound | Physical Data | (n − m) | (n = 2) | (nM) | (n = 3) | MH + (1) |
| 100 | Chemical Formula: C₄₆H₅₄F₃N₉O₈S₂ | 2 − 2 | 10.7 ± 0.3 | 9.9 | 99% | 982.3773 |
| | Exact Mass: 981.3489 | | | | | |
| | Molecular Weight: 982.1082 | | | | | |
| 101 | Chemical Formula: C₄₇H₅₆F₃N₉O₈S₂ | 2 − 3 | 14.7 ± 2.1 | 7.7 | 99% | 996.3954 |
| | Exact Mass: 995.3645 | | | | | |
| | Molecular Weight: 996.1352 | | | | | |
| 102 | Chemical Formula:C₄₇H₅₆F₃N₉O₈S₂ | 3 − 2 | 6.5 ± 0.5 | 3.3 | 99% | 996.3858 |
| | Exact Mass: 995.3645 | | | | | |
| | Molecular Weight: 996.1352 | | | | | |
| 103 | Chemical Formula: C₄₈H₅₈F₃N₉O₈S₂ | 3 − 3 | 14.5 ± 0.6 | 3.9 | 99% | 1010.3858 |
| | Exact Mass: 1009.3802 | | | | | |
| | Molecular Weight: 1010.1622 | | | | | |
| 104 | Chemical Formula: C₄₉H₆₀F₃N₉O₉S₂ | 3 − 2 − 2 | 12.7 ± 0.1 | 20.8 | 99% | 1040.4178 |
| | Exact Mass: 1039.3908 | | | | | |
| | Molecular Weight: 1040.1882 | | | | | |
| 105 | Chemical Formula:C₅₃H₆₈F₃N₉O₁₁S₂ | 3 − 2 − 2 − 2 − 2 | 6.1 ± 0.1 | 48.1 | 91% | 1128.4836 |
| | Exact Mass: 1127.4432 | | | | | |
| | Molecular Weight: 1128.2942 | | | | | |
| 106 | Chemical Formula:C₄₇H₅₆F₃N₉O₈S₂ | 3 − 2 | 11.2 ± 1.9 | N.D.* | 0% | 996.3807 |
| | Exact Mass: 995.3645 | | | | | |
| | Molecular Weight: 996.1352 | | | | | |
| 107 | Chemical Formula:C₄₁H₄₃F₃N₈O₁₀S | 3 − 3 | 11.3 ± 1.3 | 8.5 | 99% | 897.2910 |
| | Exact Mass: 896.2775 | | | | | |
| | Molecular Weight: 896.8962 | | | | | |
| 108 | Chemical Formula:C₄₂H₄₅F₃N₈O₁₁S | 3 − 2 − 2 | 4.7 ± 0.3 | 26.7 | 99% | 927.3005 |
| | Exact Mass: 926.2881 | | | | | |
| | Molecular Weight: 926.9222 | | | | | |
| 109 | Chemical Formula:C₄₆H₅₃F₃N₈O₁₃S | 3 − 2 − 2 − 2 − 2 | 9.7 ± 0.9 | 4.8 | 87% | 1015.3580 |
| | Exact Mass: 1014.3405 | | | | | |
| | Molecular Weight: 1015.0282 | | | | | |
| Defactinib | | | 3.9 ± 0.2 | N.D.* | 0% | |

*N.D. denotes that no degradation was observed.

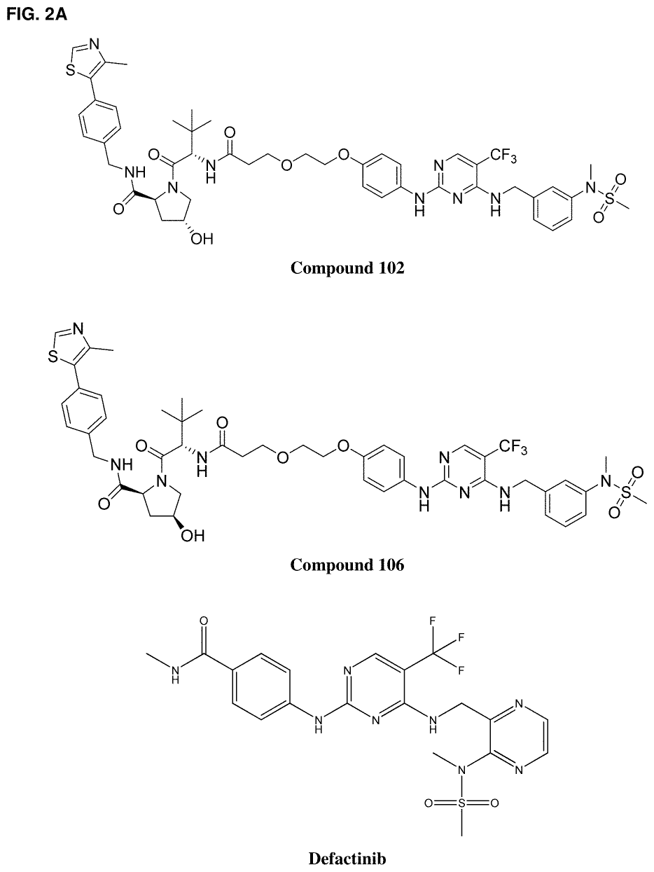

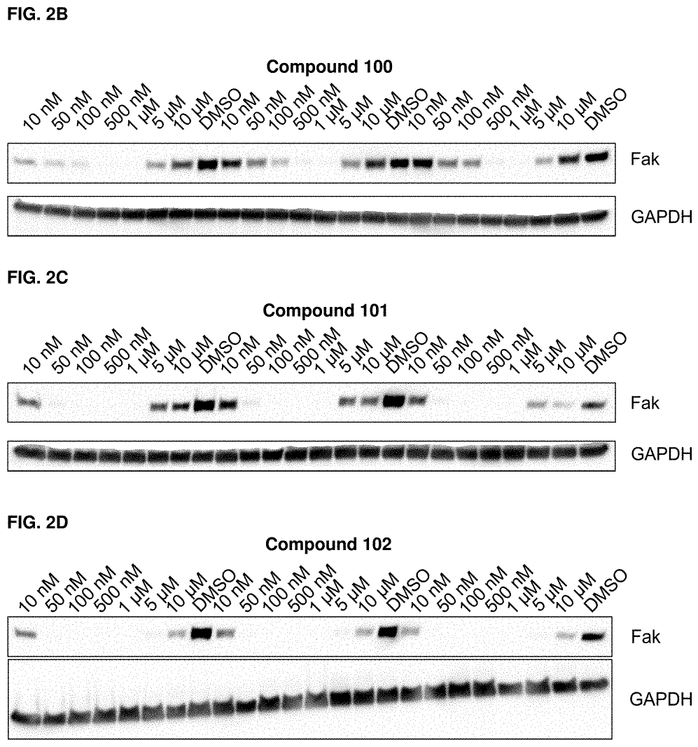

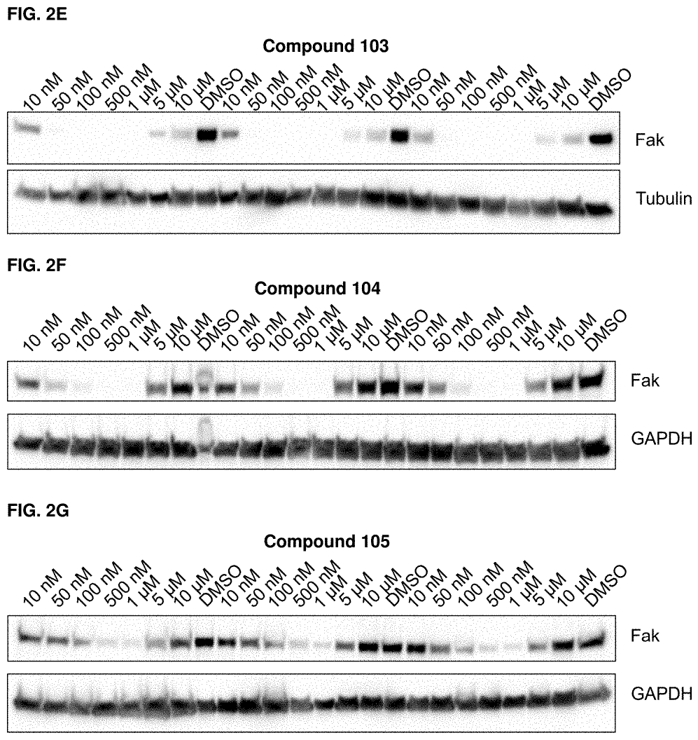

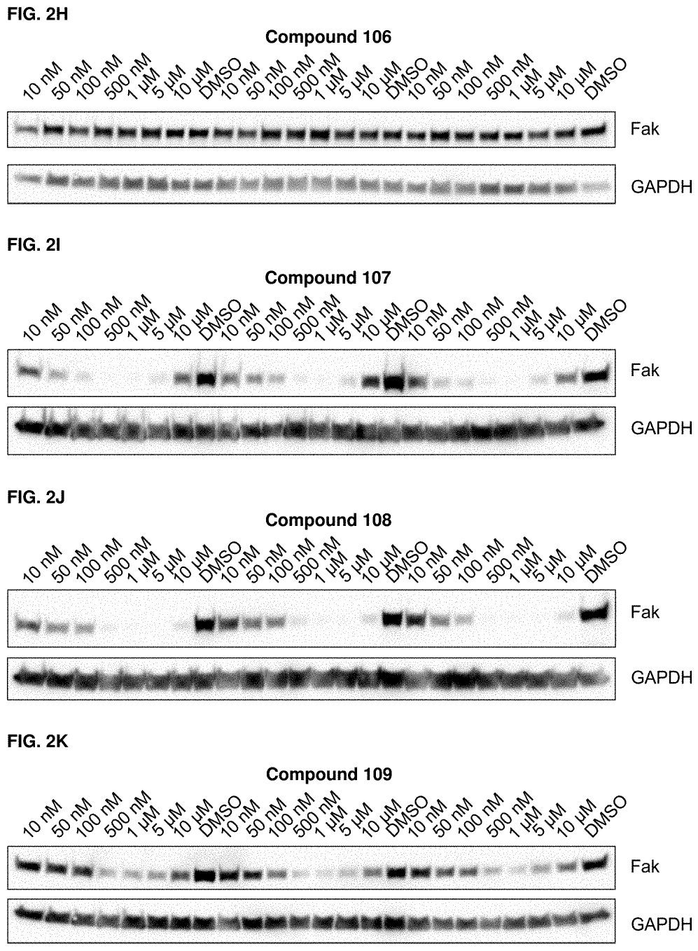

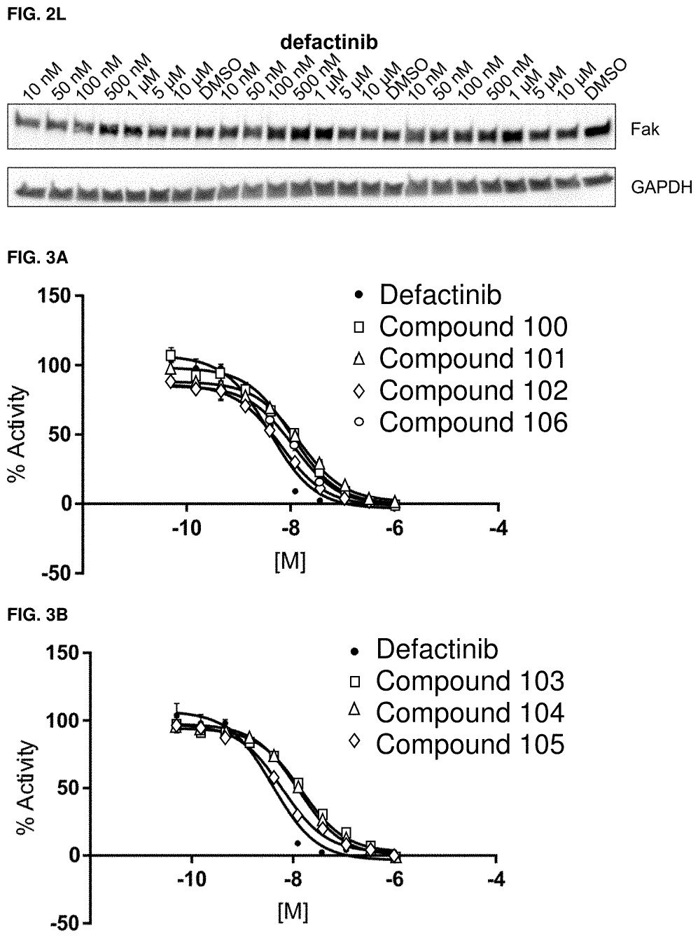

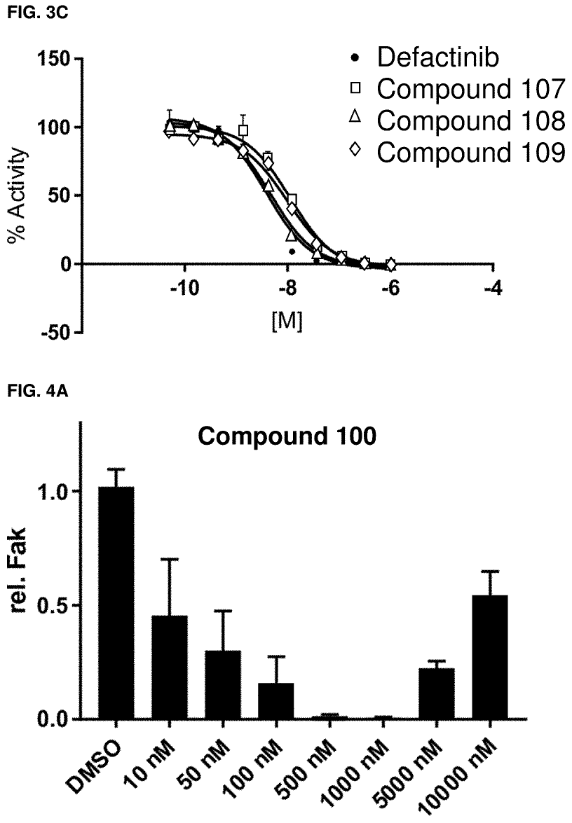

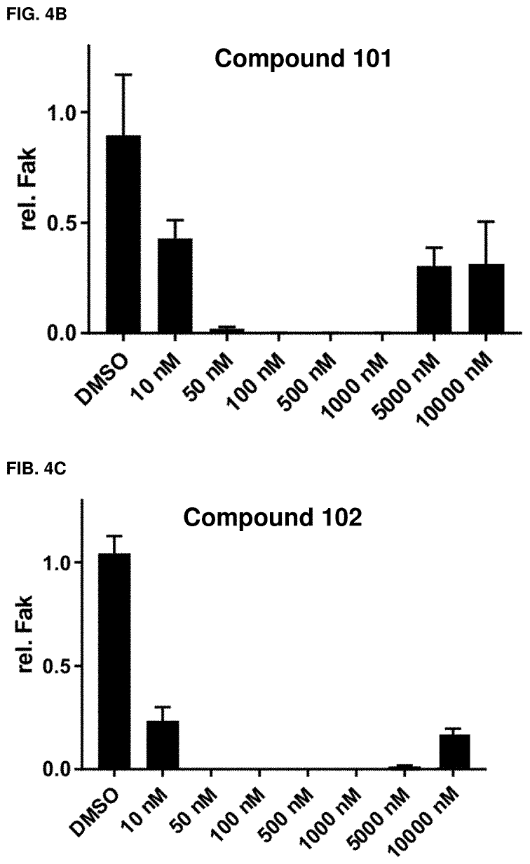

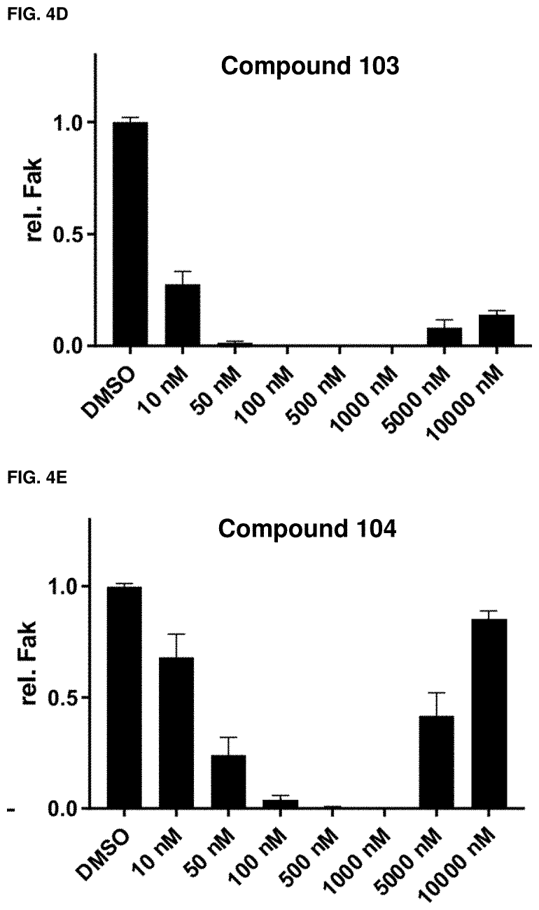

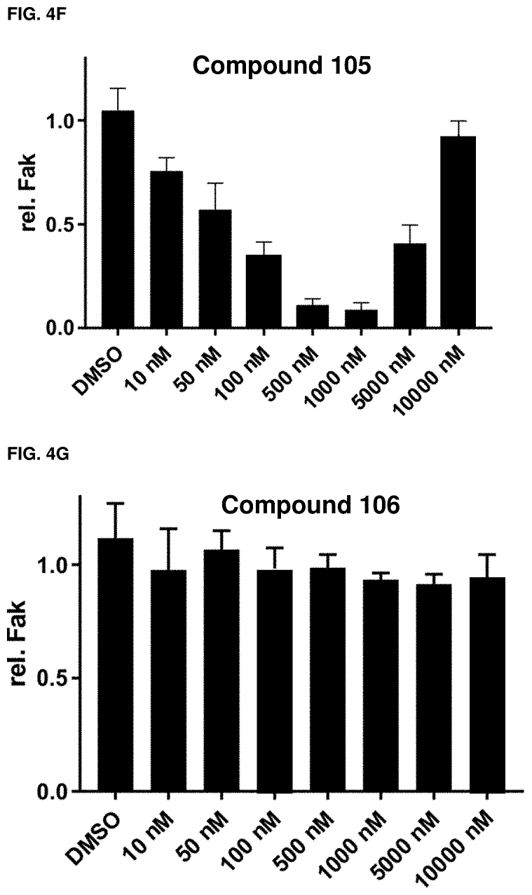

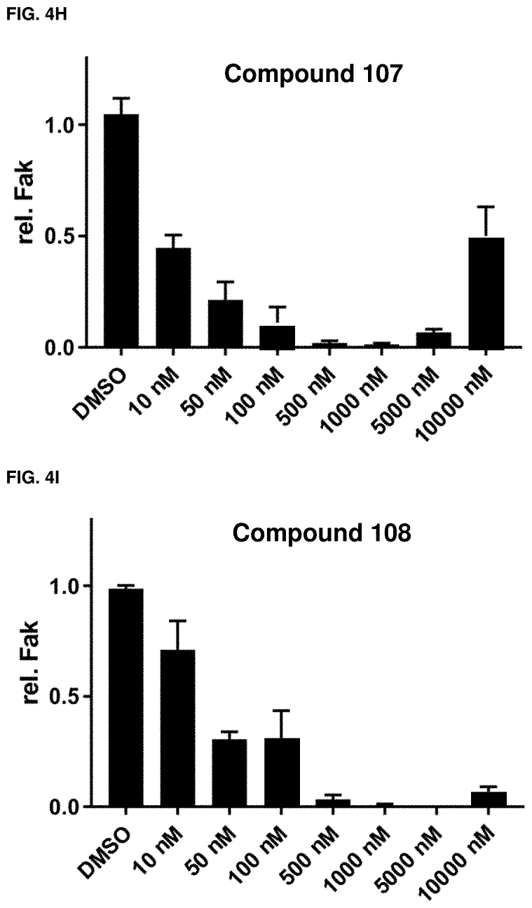

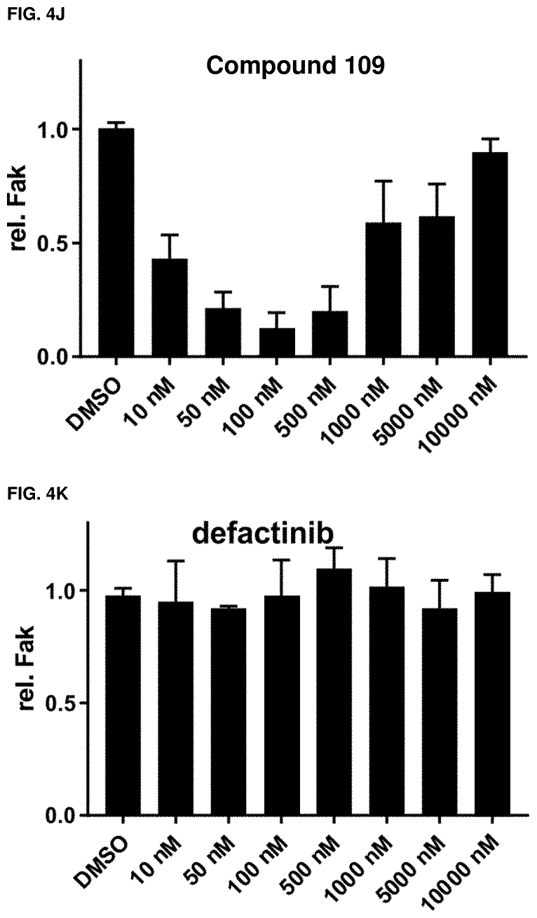

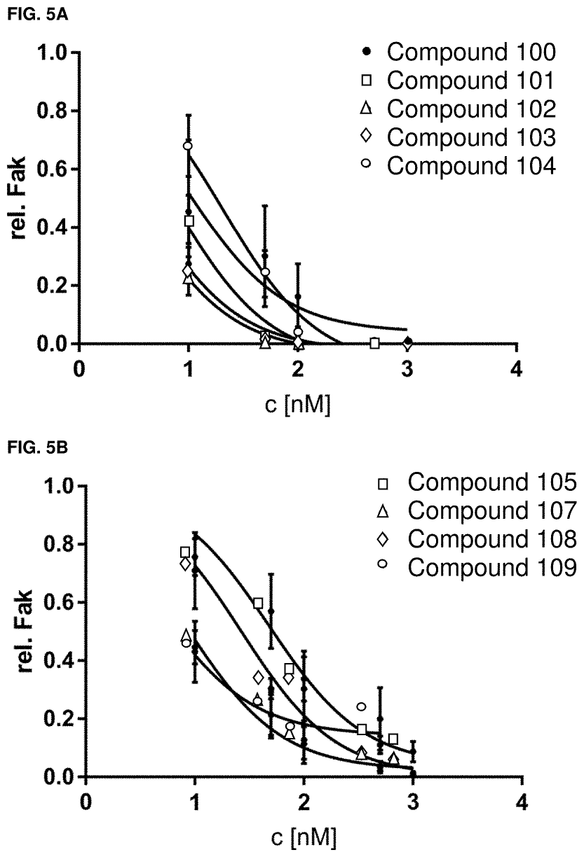

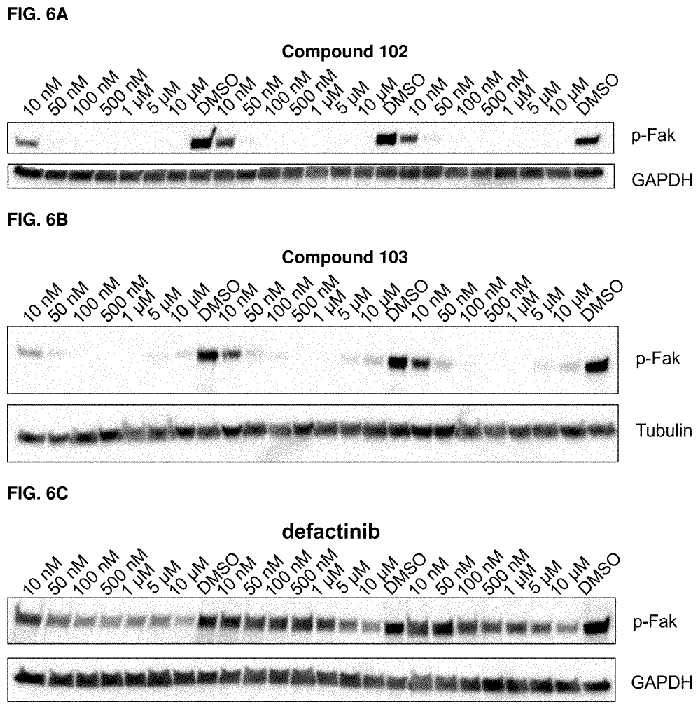

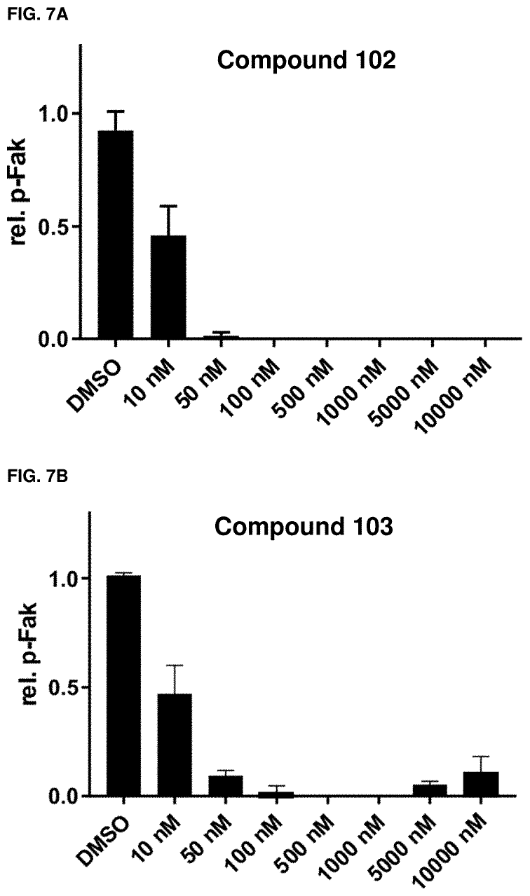

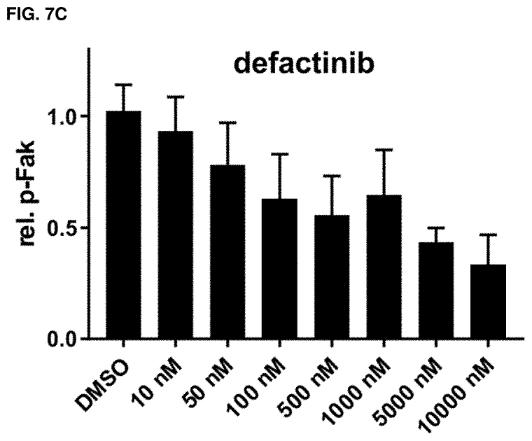

Exemplary Fak-degrading compounds were designed based on the most advanced clinical Fak inhibitor defactinib (FIG. 2A). Guided by previous SAR studies, the left part of the molecule was chosen for linker incorporation. The 4-amino-N-methylbenzamide was replaced by 4-aminophenol to facilitate linker attachment via the phenol. Due to synthetic challenges, the 2,3-substituted pyrazine was replaced by a 1,3-substituted benzyl that was previously reported to inhibit Fak with similar potency. A set of six different linkers that vary in length and composition was attached to the modified defactinib PTM (Tables 1 and 2). Coupling these different linkers with the reported VHL ligand yielded exemplar compounds 100-105 (Table 1). Based on the inhibition and degradation data, the diastereomeric exemplary compound 106 was synthesized as a negative control for exemplary compound 102. Exemplary compounds 107-109 were synthesized based on the linker composition of exemplary compounds 103-105, yet contain thalidomide as the E3 ligase recruiting element. Half-maximal inhibitory concentrations (IC₅₀) as well as half-maximal degradation concentrations (DC₅₀) and a degradation maximum (Dₘₐₓ) were calculated for exemplary compounds 100-109 and defactinib. As expected, the optimized Fak inhibitor defactinib displays the most potent IC₅₀ value (3.9 nM) of all tested compounds. Linker addition and coupling of the E3 recruiting element to this inhibitor does not have a major effect on Fak inhibition and no general trend was observed. All exemplary compounds of the present disclosure inhibit Fak kinase activity with low nanomolar IC₅₀s between 4.7 nM and 14.5 nM (Table 2, FIGS. 3A-3C). However, as already observed in previous studies, inhibition and degradation do not always correlate. For example, the best Fak-inhibiting exemplary compound, compound 108, is one of the least potent degraders (DC₅₀ 26.7 nM). On the contrary, exemplar compound 103 combines the least potent IC₅₀ (14.5 nM) with the second most potent DC₅₀ (3.9 nM). Inversion of the hydroxyproline stereo center on exemplary compound 106 (IC₅₀=11.2 nM) results in a minimal loss of potency compared to its diastereomer exemplary compound 102 (IC₅₀=6.5 nM). The maximum degradation efficacy (Dₘₐₓ) for most exemplary compounds is at the limit of detection (99%) (FIGS. 2B-2L, 4A-4K, 5A and 5B); only the two compounds containing the longest linkers exemplar compound 105 and exemplar compound 109 show slightly reduced Dₘₐₓ of 91% and 87%, respectively. As expected, the negative control molecules, defactinib and the non VHL binding diastereomer exemplar compound 106 induce no Fak degradation. As a general trend, VHL-recruiting exemplary compounds 100-105 appear to be more effective degraders than their CRBN-recruiting analogs exemplar compounds 107-109. In addition, linkers that are too long (exemplar compounds 104 and 105) or too short (exemplar compound 100) yield less effective compounds with DC₅₀s of 20.8 nM, 48.1 nM and 9.9 nM, respectively. A three carbon linkage on the VHL ligand appears to be preferred over a two carbon linkage: exemplar compound 102 and exemplar compound 103 display almost identical DC₅₀ values of 3.3 nM and 3.9 nM, respectively, combined with an excellent Dₘₐₓ of 99%, whereas exemplar compound 101 is slightly less potent with a DC₅₀ of 7.7 nM. As exemplary compound 102 shows very efficient Fak degradation (FIG. 2D), has the slightly better DC₅₀ and displays a stronger suppression of p-Fak(Y397) levels (FIGS. 6A-6C and 7A-7C) it was selected for all further characterization.

Examination of Target Protein Selectivity. To assess the target selectivity of exemplary compound 102 over a large panel of different kinases, a DiscoverX KINOMEScan was performed. KINOMEScan measures compound binding to individual kinases via the compounds ability to compete/displace the kinases from an immobilized support that non-selectively binds kinase active sites. Defactinib (1 μM) binds to 100 kinases such that less than 35% of the control (uncompeted) level of kinase remain attached to the support. However, exemplary compound 102 shows highly increased selectivity as it binds only 20 kinases to a comparable extent under identical conditions (Table 3). Surprisingly, Fak is the only kinase bound by exemplary compound 102 with less than 1% of control remaining, whereas defactinib binds a total of 9 kinases to this extent (Table 3). It appears that the slight loss in inhibitory potency due to linker and VHL ligand attachment results in greater selectivity.

TABLE 3
Selectivity scores of Exemplary Compound 102 and defactinib at
1 μμM compound concentration

| | | | Number | | |
|---|---|---|---|---|---|
| | Selectivity | | of Non- | Screening | |
| Compound | Score | Number | Mutant | Concentration | Selectivity |
| Name | Type | of Hits | Kinases | (nM) | Score |
| Defactinib | S(35) | 100 | 403 | 1000 | 0.248 |
| Defactinib | S(10) | 39 | 403 | 1000 | 0.097 |
| Defactinib | S(1) | 9 | 403 | 1000 | 0.022 |
| 102 | S(35) | 20 | 403 | 1000 | 0.05 |
| 102 | S(10) | 7 | 403 | 1000 | 0.017 |
| 102 | S(1) | 1 | 403 | 1000 | 0.002 |

Examination of Downstream Signaling Effects of Fak Degradation.

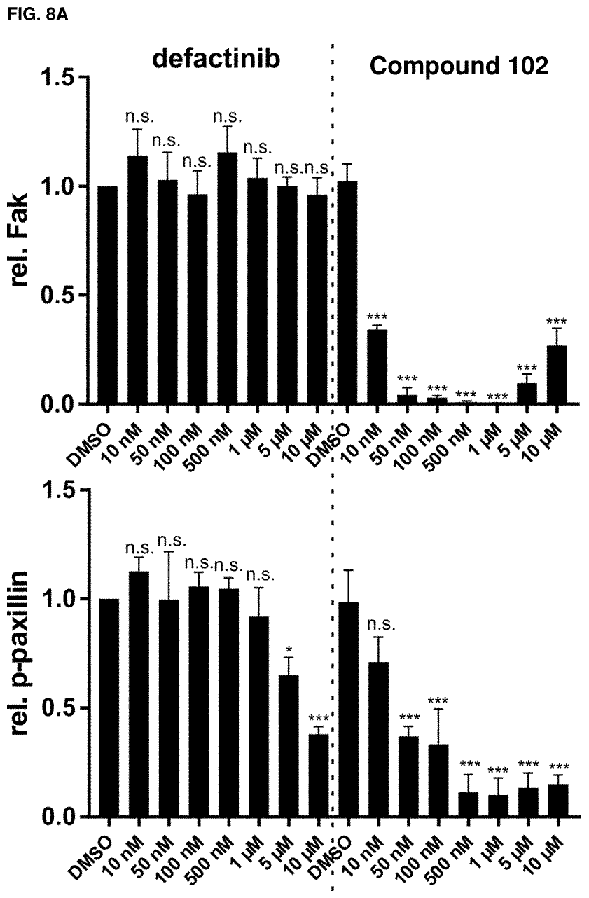

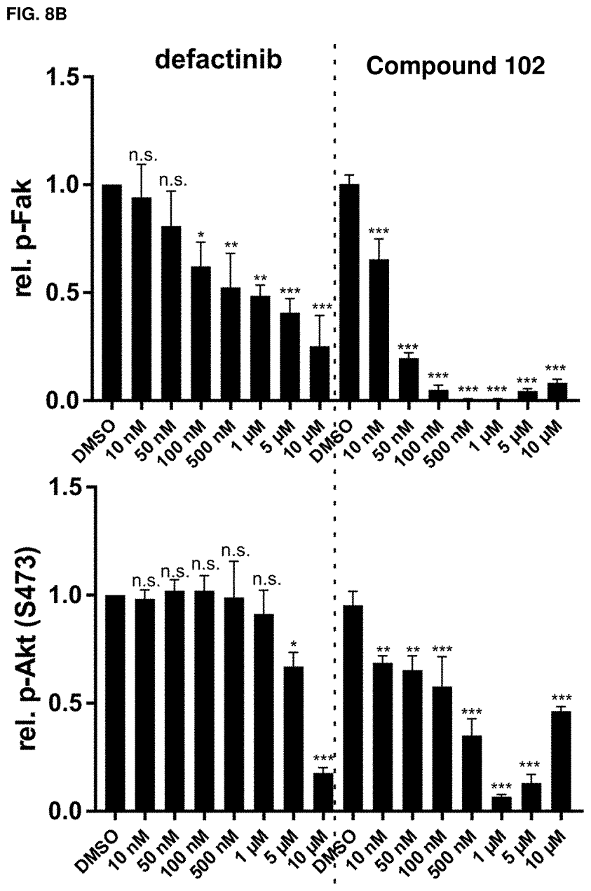

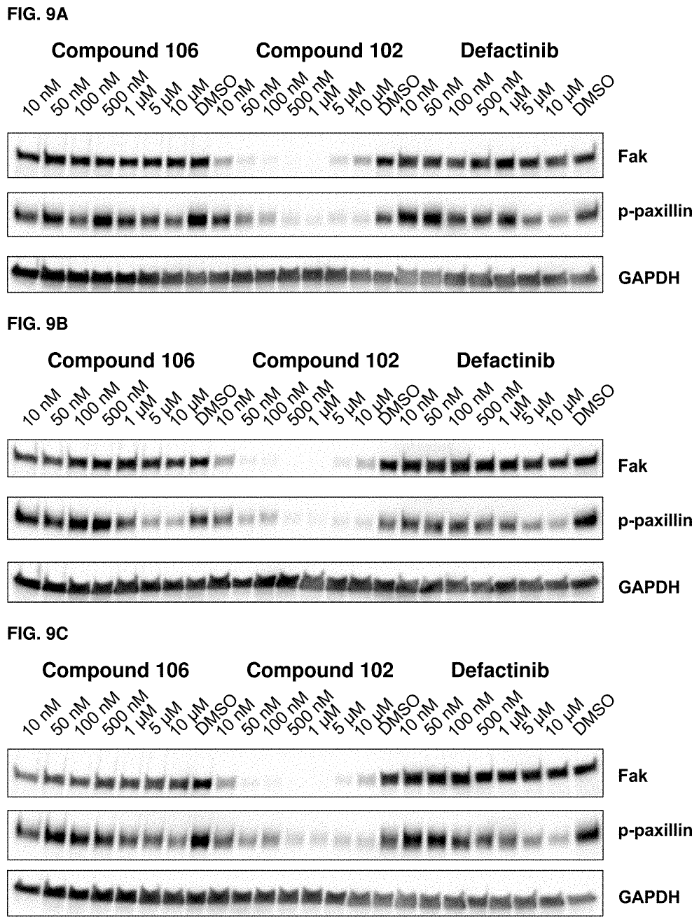

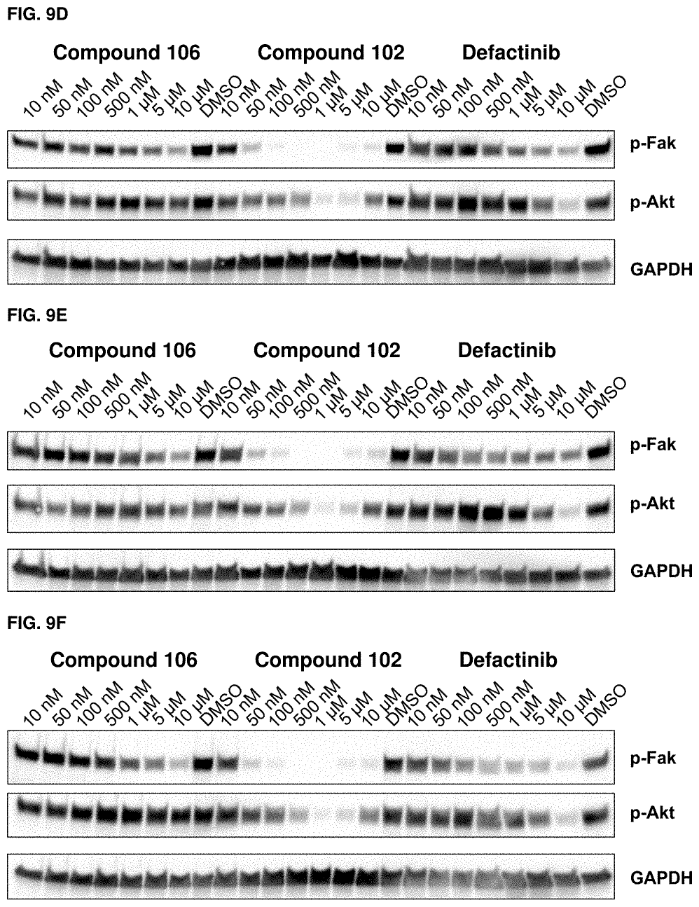

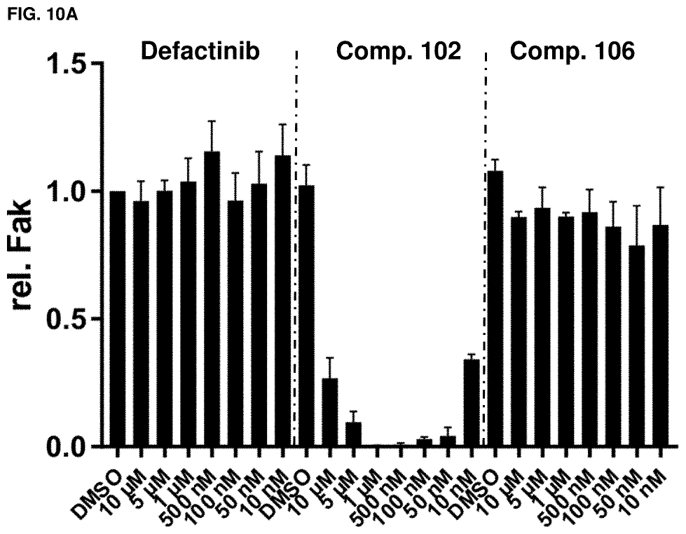

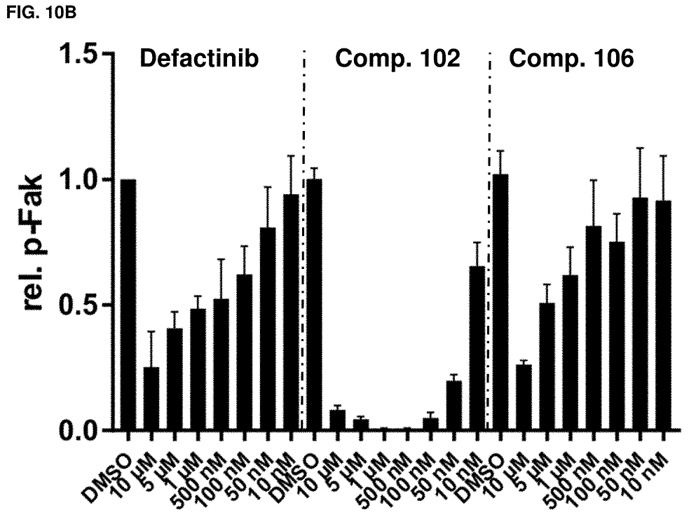

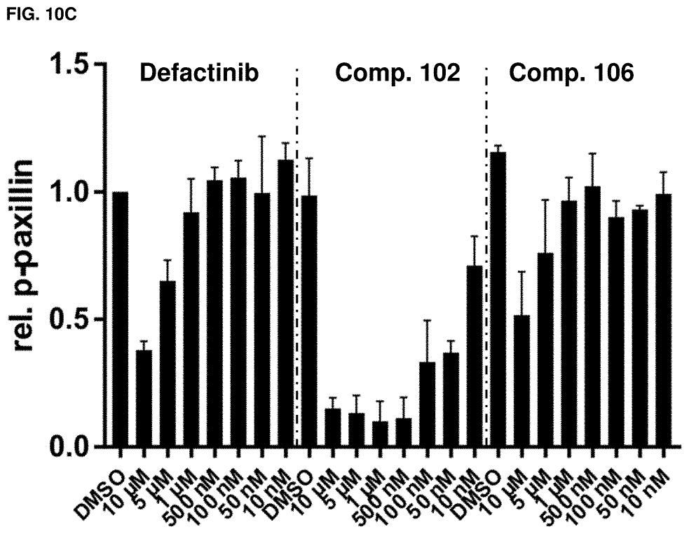

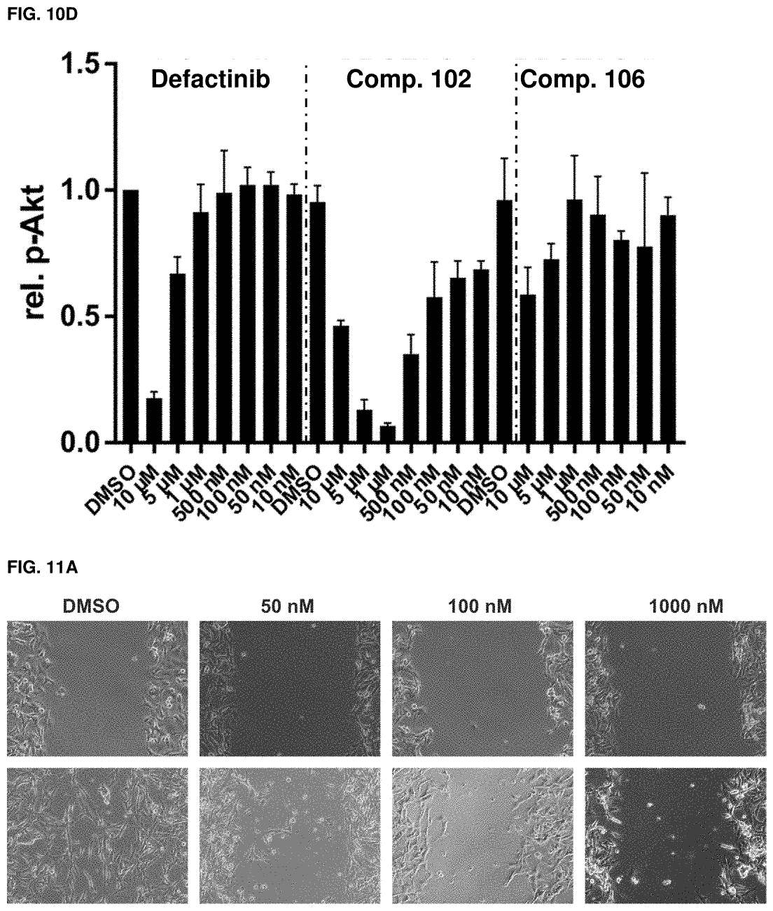

To evaluate the benefits of Fak degradation over inhibition on downstream signaling, a head-to-head comparison between exemplar compound 102 and defactinib was performed (FIGS. 8A-8D, 9A-9F, and 10A-10D). Human prostate tumor (PC3) cells were treated with increasing concentrations of exemplary compound 102 and defactinib and cellular effects were evaluated via western blotting for total Fak levels, Fak activity (autophosphorylation of Y397) as well as phosphorylation of two downstream targets of Fak: paxillin and Akt. As already evident in Table 2, exemplary compound 102 induces highly efficient Fak degradation in a dose-dependent manner with only 34% total Fak remaining at 10 nM and 5% at 50 nM (FIG. 10A). Fak levels are undetectable at concentrations of 100 nM through 1 μM of exemplary compound 102, and slightly rebound at concentrations of 5 μM (10%) and 10 μM (27%) due to an observed hook effect. In contrast, incubation with defactinib does not show any effect on Fak levels. Fak activation (p-Fak(Y397)) was significantly reduced at all exemplary compound 102 concentrations tested compared to DMSO: p-Fak levels of less than 5% were observed between 100 nM and 5 μM (FIG. 10B). Defactinib showed significantly reduced Fak activity only at concentrations above 100 nM, and at no concentration was defactinib able to outperform exemplary compound 102 with respect to p-Fak loss. The lowest level of p-Fak activity (26% remaining) was observed with 10 μM defactinib treatment, a concentration at which the inhibitor is predicted to show a high level of off-target activity (KINOMEScan). Paxillin, a downstream target of the Fak-Src complex, has been associated with cell migration. Paxillin interacts with the FAT domain and reduced levels of Fak result in a reduction of p-paxillin. Exemplary compound 102 treatment above 50 nM is able to significantly reduce p-paxillin levels by as much as 85-90% (FIG. 10C). Defactinib, on the other hand, reduces p-paxillin levels by a maximum of only 62%, and then solely at the high concentration of 10 μM. Akt is a kinase that is tied to the Fak signaling cascade via PI3K, but can be activated through other pathways as well. Consequently, the suppressive effect of exemplary compound 102 on p-Akt (S473) are not as pronounced as for paxillin and Fak, but nonetheless still significant at all treatment concentrations (FIG. 10D). A maximum p-Akt suppression of 93% is observed at 1 μM of exemplary compound 102. Conversely, defactinib shows no reduction of p-Akt at concentrations below 5 μM, and has a maximum reduction of p-Akt at 10 μM (88%). Judging by the high number of bound kinases at 1 μM (100 kinases, Table 3), it is very possible that the observed effects at 5 μM and 10 μM defactinib may be due to off-target binding. Evaluating the activation profile in FIGS. 10A-10D it is clear that exemplary compound 102-mediated Fak degradation has a more pronounced effect on the effector targets within the Fak signaling pathway compared to the clinical candidate defactinib. A similar differential can be observed when exemplary compound 102 is compared to its non-degrading diastereomer exemplary compound 106 (FIGS. 9A-9F and 10A-10D). These differences are the result of the distinct MOA Fak degraders are able to provide compared to inhibitors.

Examination of Cell Migration and Invasion.

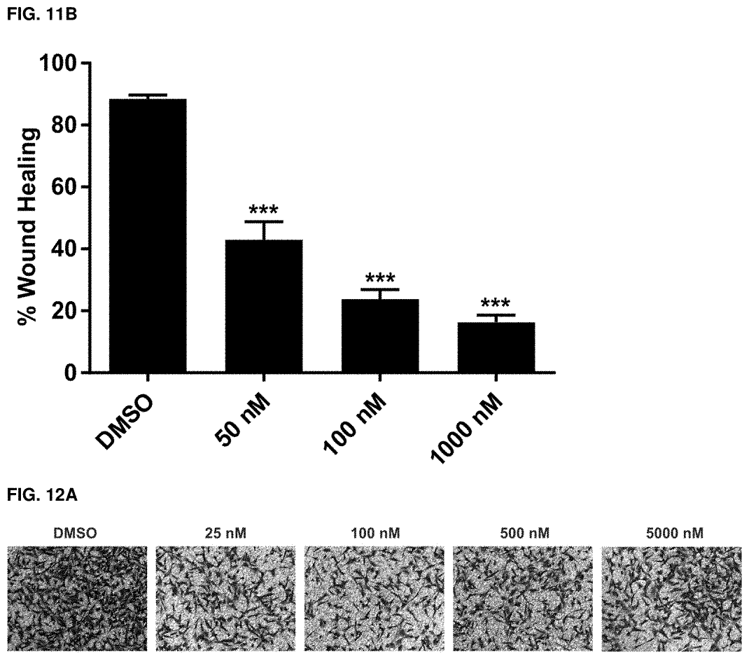

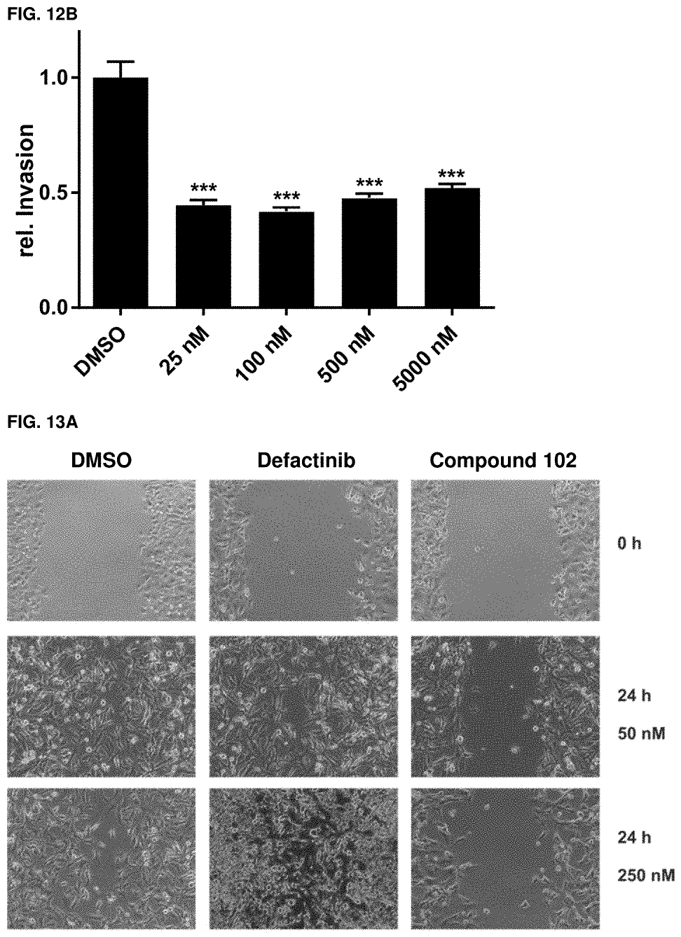

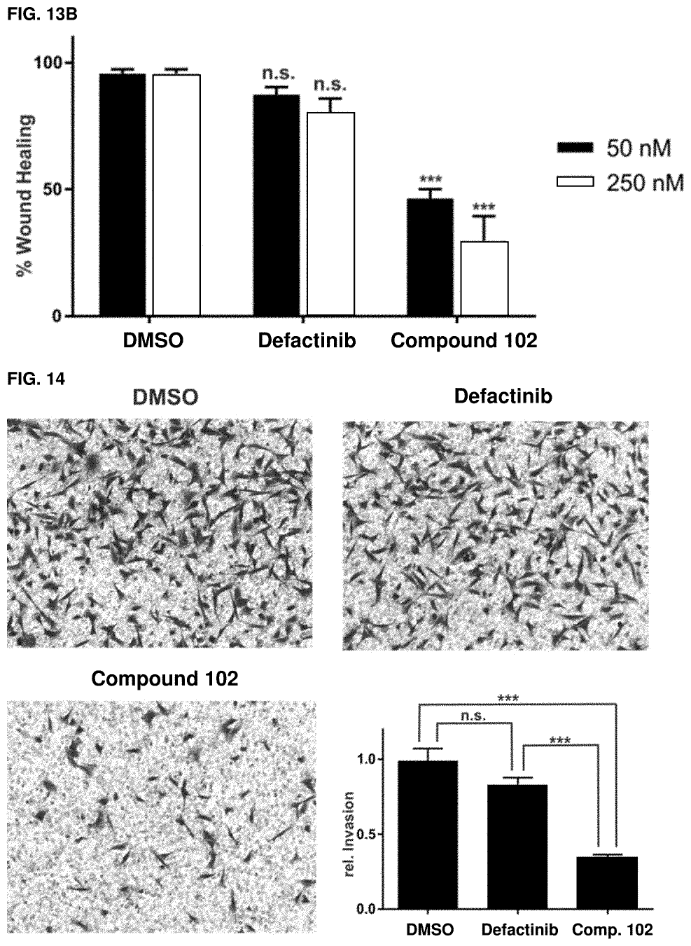

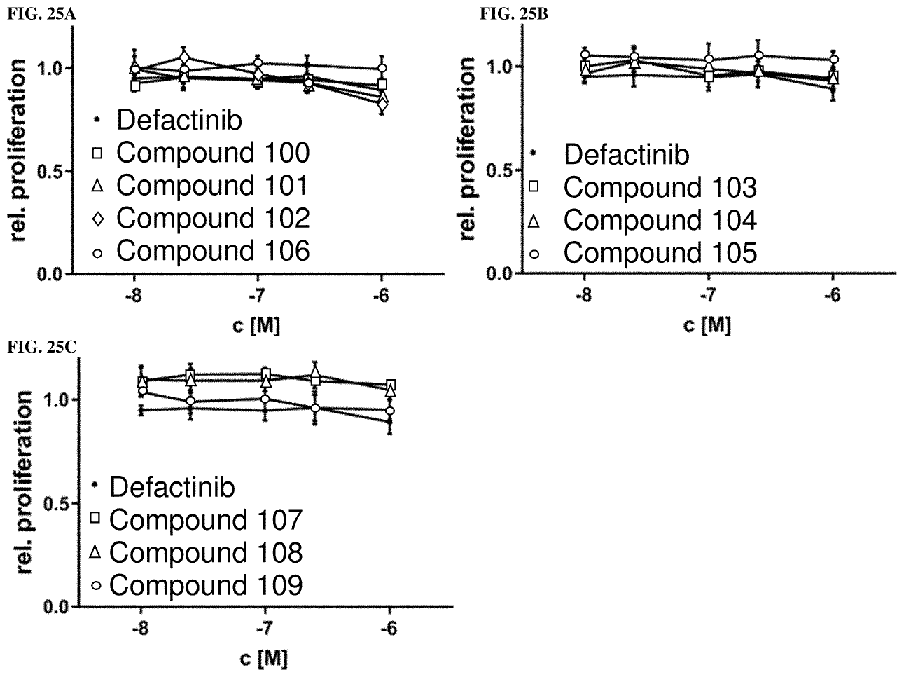

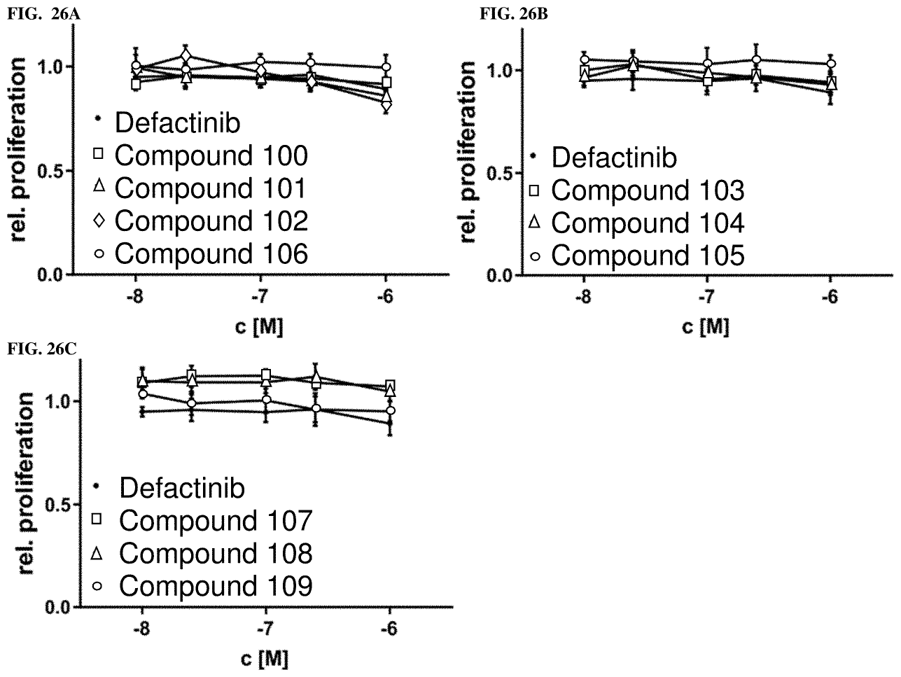

Since Fak is a key regulator of cell motility, exemplary compound 102 was evaluated for its effect on cell migration and invasion. Despite their previously described effects on Fak activation and signaling, exemplary compound 102 and defactinib do not affect cell viability or proliferation within four days (FIGS. 25A-25C, 26A-26C, 11A, 11B, 12A, and 12B). Effects on cell migration were analyzed in a wound healing assay using the aggressive and invasive human TNBC cell line MDA-MB-231. MDA-MD-231 cells were grown to confluency and a wound was created using a pipet tip. Wound closure was quantified after 24 hours (FIGS. 13A and 13B). While near-complete wound closure can be observed after 24 hours in cultures treated with 50 nM defactinib or vehicle equivalent (DMSO), treatment with 50 nM exemplary compound 102 significantly impairs cell migration and results in a 53% reduction of wound healing. Moreover, treatment with 250 nM of exemplary compound 102 further impairs wound closure by 70% (FIGS. 13B, 11A and 11B), while 250 nM defactinib treatment results in a non-significant suppression of wound healing. Since exemplary compound treatment did not affect cell proliferation at the concentrations applied, the observed effects result from reduced migratory properties of cancer cells due to Fak degradation.

To diminish the contribution of cell growth, a transwell cell invasion assay was performed (FIG. 14). MDA-MB-231 cells were treated with exemplary compound 102 or defactinib at 100 nM and transwell migration was quantified after 24 hours (FIGS. 14,12A and 12B). While exemplary compound 102 reduces MDA-MB-231 cell invasion by as much as 65%, no significant effect is observed for defactinib or DMSO. Treatment with exemplary compound 102 significantly impairs cell invasion compared to defactinib, underscoring the importance of Fak's scaffolding function in the context of cell migration and invasion.

Examination of the Molecular Signaling Events) and/or Downstream Pathways Responsible for the Migration and Invasion Effects Observed by Exemplary Compound Treatment. To pinpoint these observations to a molecular signaling event or specific downstream pathway, reverse phase protein array (RPPA) analysis was performed (Table 4).

TABLE 4
RPPA results. Relative protein levels after incubation of MDA-MB-231 cells with
DMSO (0.1%), exemplary compound 102 (500nM) or defactinib (1000nM). n = 2; error = SD.

| | DMSO % | | Compound 102 | | defactinib | |
|---|---|---|---|---|---|---|
| | rel. Protein | SD | rel. Protein | SD | rel. Protein | SD |
| Antibody Name | level [%] | | level [%] | | level [%] | |
| 14-3-3-zeta | 97.6 | 1.9 | 105.4 | 9.3 | 102.9 | 3.4 |
| 14-3-3-beta | 99.6 | 2.6 | 100.5 | 1.1 | 98.4 | 0.2 |
| 4E-BP1 | 97.9 | 3.0 | 110.6 | 0.3 | 115.6 | 15.7 |
| 4E-BP1_pS65 | 110.6 | 3.2 | 92.3 | 2.6 | 91.8 | 0.6 |
| 53BP1 | 94.2 | 2.9 | 111.3 | 2.0 | 111.6 | 9.5 |
| A-Raf | 98.1 | 2.7 | 106.5 | 3.0 | 108.3 | 5.3 |
| ACC1 | 100.0 | 0.1 | 103.3 | 9.5 | 95.9 | 10.9 |
| ACC_pS79 | 111.5 | 16.3 | 95.4 | 5.6 | 102.5 | 2.9 |
| ADAR1 | 106.4 | 15.6 | 121.2 | 7.4 | 111.8 | 10.3 |
| Akt | 96.4 | 2.1 | 101.7 | 2.9 | 99.2 | 8.5 |
| Akt_pS473 | 96.1 | 5.5 | 74.2 | 8.8 | 79.3 | 8.9 |
| Akt_pT308 | 112.7 | 7.4 | 94.2 | 0.2 | 97.2 | 1.0 |
| AMPK-a2_pS345 | 103.6 | 5.1 | 97.2 | 2.6 | 98.1 | 1.2 |
| AMPKa | 99.6 | 0.6 | 105.1 | 7.2 | 105.6 | 7.4 |
| AMPKa_pT172 | 110.4 | 2.6 | 92.7 | 3.1 | 101.3 | 4.6 |
| Annexin-I | 106.5 | 8.4 | 117.8 | 11.1 | 96.1 | 9.1 |
| Annexin-VII | 102.7 | 3.8 | 110.0 | 1.1 | 107.8 | 7.7 |
| AR | 118.8 | 16.0 | 85.0 | 26.3 | 103.1 | 1.5 |
| ARID1A | 96.4 | 22.2 | 121.1 | 5.4 | 100.8 | 16.4 |
| Atg3 | 98.0 | 2.9 | 98.3 | 0.2 | 98.1 | 3.1 |
| Atg7 | 95.6 | 1.6 | 100.8 | 1.2 | 99.5 | 1.8 |
| ATM | 97.7 | 1.9 | 106.8 | 0.3 | 93.9 | 3.7 |
| ATM_pS1981 | 99.9 | 0.2 | 99.7 | 0.2 | 99.4 | 1.2 |
| ATR_pS428 | 101.5 | 2.1 | 99.2 | 0.7 | 93.7 | 1.4 |
| Aurora-B | 104.5 | 5.1 | 92.3 | 2.6 | 91.9 | 4.2 |
| Axl | 100.3 | 0.4 | 94.7 | 3.3 | 76.0 | 2.8 |
| b-Actin | 97.3 | 3.9 | 97.4 | 0.2 | 101.1 | 1.3 |
| b-Catenin | 90.2 | 2.8 | 108.4 | 11.4 | 100.0 | 10.5 |
| b-Catenin_pT41_S45 | 127.0 | 4.1 | 96.8 | 0.5 | 98.3 | 2.6 |
| B-Raf | 102.0 | 3.5 | 110.9 | 5.2 | 104.8 | 2.2 |
| B-Raf_pS445 | 99.3 | 1.0 | 108.0 | 4.4 | 108.8 | 9.7 |
| B7-H4 | 98.8 | 1.7 | 97.5 | 2.6 | 102.6 | 2.8 |
| Bad_pS112 | 101.4 | 2.0 | 102.3 | 3.2 | 103.3 | 2.9 |
| Bak | 101.2 | 1.8 | 100.7 | 1.2 | 103.1 | 0.5 |
| BAP1 | 98.5 | 2.2 | 96.8 | 1.6 | 83.5 | 8.5 |
| Bax | 101.0 | 1.4 | 110.5 | 4.2 | 106.4 | 2.7 |
| Bc1-xL | 98.9 | 1.3 | 97.6 | 0.9 | 100.4 | 4.7 |
| Bc12 | 100.8 | 1.1 | 92.8 | 1.8 | 112.9 | 19.7 |
| Beclin_l | 100.4 | 2.9 | 100.0 | 2.3 | 99.2 | 2.3 |
| Bid | 98.1 | 2.7 | 102.8 | 5.6 | 109.2 | 6.5 |
| Bim | 101.8 | 2.6 | 102.9 | 0.3 | 105.6 | 6.6 |
| BiP-GRP78 | 99.3 | 1.0 | 103.0 | 0.3 | 103.7 | 1.2 |
| BRD4 | 102.9 | 4.2 | 118.7 | 14.1 | 108.7 | 14.3 |
| c-Abl | 97.9 | 5.1 | 95.2 | 0.4 | 109.1 | 9.9 |
| c-IAP2 | 92.8 | 0.8 | 100.4 | 5.1 | 94.3 | 3.1 |
| c-Jun_pS73 | 132.6 | 3.9 | 92.3 | 2.6 | 92.7 | 3.3 |
| c-Kit | 102.7 | 3.9 | 100.1 | 3.6 | 101.5 | 3.5 |
| c-Met_pY1234_Y1235 | 103.0 | 4.2 | 101.1 | 1.2 | 98.9 | 1.8 |
| c-Myc | 128.8 | 10.7 | 89.2 | 2.1 | 95.9 | 0.8 |
| C-Raf | 94.9 | 0.5 | 100.8 | 0.8 | 96.5 | 3.4 |
| C-Raf_pS338 | 108.7 | 8.2 | 90.7 | 3.8 | 98.1 | 2.3 |
| Caspase-3 | 100.3 | 0.5 | 106.7 | 7.0 | 108.3 | 5.3 |
| Caspase-7-cleaved- | 108.1 | 11.4 | 113.1 | 2.2 | 107.0 | 3.4 |
| Caspase-8 | 103.2 | 1.2 | 102.4 | 4.1 | 97.2 | 1.1 |
| Caveolin-1 | 97.4 | 3.6 | 141.0 | 19.5 | 135.0 | 43.1 |
| CD134 | 102.9 | 0.0 | 99.0 | 4.7 | 100.5 | 5.7 |
| CD20 | 102.2 | 0.5 | 92.3 | 2.6 | 96.8 | 0.2 |
| CD29 | 99.2 | 1.1 | 104.7 | 3.2 | 101.9 | 1.5 |
| CD31 | 105.2 | 7.3 | 98.2 | 6.4 | 100.7 | 5.4 |
| CD4 | 98.4 | 2.2 | 101.8 | 3.1 | 103.7 | 1.2 |
| CD44 | 105.5 | 3.5 | 106.6 | 12.1 | 100.5 | 5.7 |
| CD45 | 97.7 | 3.3 | 97.9 | 4.6 | 117.4 | 18.1 |
| CD49b | 107.3 | 10.4 | 116.5 | 12.4 | 105.5 | 1.3 |
| cdc25C | 92.8 | 0.9 | 99.5 | 0.7 | 98.2 | 3.0 |
| cdc2_pY15 | 103.7 | 3.2 | 92.3 | 2.6 | 90.1 | 6.2 |
| CDK1_pT14 | 99.6 | 0.5 | 94.6 | 7.4 | 84.4 | 7.8 |
| Chkl | 104.0 | 0.5 | 92.3 | 2.6 | 84.1 | 8.0 |
| Chkl_pS296 | 106.1 | 2.7 | 96.6 | 0.7 | 98.2 | 2.4 |
| Chk2 | 99.7 | 5.3 | 94.4 | 4.6 | 97.0 | 0.7 |
| Chk2_pT68 | 99.3 | 0.9 | 103.4 | 0.0 | 106.0 | 2.1 |
| Claudin-7 | 94.1 | 4.7 | 101.6 | 1.2 | 97.3 | 1.2 |
| COG3 | 101.8 | 2.6 | 107.9 | 3.5 | 105.5 | 1.3 |
| Collagen-VI | 98.4 | 2.3 | 93.4 | 0.4 | 102.5 | 6.6 |
| Connexin-43 | 105.2 | 6.0 | 92.3 | 2.6 | 86.5 | 4.4 |
| Cox-IV | 91.6 | 0.9 | 104.5 | 4.4 | 101.8 | 5.7 |
| Cox2 | 96.1 | 5.5 | 171.3 | 14.2 | 144.9 | 10.2 |
| Creb | 103.9 | 5.5 | 103.4 | 3.0 | 103.7 | 3.1 |
| Cyclin-B1 | 98.6 | 2.0 | 96.7 | 3.7 | 86.8 | 8.2 |
| Cyclin-D3 | 102.8 | 3.9 | 94.7 | 4.6 | 97.2 | 0.5 |
| Cyclin-E1 | 100.8 | 1.9 | 110.5 | 8.8 | 101.8 | 4.5 |
| Cyclin-D1 | 103.0 | 4.1 | 105.2 | 3.5 | 100.5 | 5.7 |
| Cyclophilin-F | 96.1 | 5.5 | 97.6 | 3.4 | 85.5 | 0.1 |
| D-a-Tubulin | 101.6 | 1.4 | 95.2 | 4.1 | 98.2 | 2.4 |
| DJ1 | 95.5 | 3.1 | 103.5 | 2.0 | 105.3 | 6.7 |
| DM-Histone-H3 | 97.7 | 3.2 | 108.5 | 6.6 | 106.7 | 3.1 |
| DM-K9-Histone-H3 | 95.9 | 2.7 | 111.9 | 1.2 | 108.9 | 6.2 |
| DUSP4 | 89.6 | 3.6 | 142.6 | 8.4 | 135.6 | 22.0 |
| E-Cadherin | 86.9 | 7.5 | 92.7 | 5.7 | 122.5 | 5.7 |
| E2F1 | 99.1 | 1.2 | 98.2 | 1.6 | 98.4 | 0.6 |
| eEF2 | 96.4 | 0.7 | 98.9 | 0.6 | 94.8 | 4.2 |
| eEF2K | 95.7 | 5.0 | 110.8 | 4.6 | 116.4 | 12.3 |
| EGFR | 99.2 | 1.1 | 98.2 | 2.7 | 93.7 | 7.0 |
| EGFR_pY1173 | 101.4 | 2.0 | 101.5 | 5.4 | 100.8 | 1.7 |
| eIF4E | 101.1 | 1.6 | 102.9 | 1.5 | 105.5 | 2.8 |
| eIF4E_pS209 | 96.4 | 3.4 | 95.8 | 1.4 | 95.7 | 1.7 |
| eIF4G | 95.0 | 2.7 | 96.7 | 3.7 | 95.8 | 7.9 |
| Elk1_pS383 | 100.7 | 0.9 | 97.5 | 0.7 | 97.3 | 1.0 |
| EMA | 96.0 | 1.2 | 109.4 | 9.0 | 102.2 | 5.1 |
| ENY2 | 95.9 | 0.1 | 95.1 | 1.4 | 95.1 | 2.0 |
| ER | 102.3 | 3.2 | 98.1 | 6.1 | 99.6 | 3.1 |
| ER-a_pS118 | 96.3 | 5.2 | 109.9 | 17.5 | 98.5 | 8.5 |
| ERCC1 | 99.5 | 0.6 | 93.6 | 0.7 | 85.6 | 2.4 |
| ERCC5 | 96.9 | 4.4 | 103.3 | 3.3 | 110.3 | 8.2 |
| Ets-1 | 107.0 | 0.5 | 97.7 | 0.7 | 90.6 | 2.8 |
| FAK | 106.2 | 8.2 | 55.4 | 8.6 | 116.1 | 12.4 |
| FAK_pY397 | 136.7 | 0.2 | 86.4 | 1.0 | 93.5 | 1.6 |
| FASN | 99.2 | 3.3 | 92.6 | 2.1 | 98.1 | 1.9 |
| Fibronectin | 98.2 | 8.5 | 103.9 | 19.0 | 89.9 | 15.4 |
| FOXM1 | 98.2 | 2.5 | 92.4 | 2.4 | 90.3 | 2.6 |
| FoxO3a | 99.6 | 0.0 | 100.5 | 1.6 | 104.9 | 0.5 |
| FoxO3a_pS318_S321 | 101.4 | 1.9 | 96.0 | 1.4 | 99.0 | 0.8 |
| G6PD | 96.6 | 2.6 | 97.7 | 3.9 | 102.3 | 5.3 |
| Gab2 | 91.7 | 0.7 | 100.5 | 0.4 | 94.6 | 3.1 |
| GAPDH | 84.2 | 22.2 | 99.4 | 0.8 | 104.0 | 10.6 |
| GATA-6 | 103.3 | 2.0 | 99.4 | 4.8 | 99.4 | 4.1 |
| GATA3 | 102.0 | 2.9 | 97.2 | 4.4 | 91.1 | 2.3 |
| GCLM | 99.1 | 1.1 | 99.9 | 0.1 | 97.0 | 1.3 |
| GCN5L2 | 100.2 | 0.3 | 99.4 | 0.2 | 94.5 | 0.2 |
| Glutamate-D1-2 | 98.9 | 0.3 | 95.5 | 2.1 | 100.6 | 1.6 |
| Glutaminase | 112.5 | 17.7 | 111.5 | 19.8 | 100.6 | 5.3 |
| Granzyme-B | 101.6 | 6.2 | 101.4 | 2.0 | 102.7 | 0.3 |
| GSK-3a-b | 95.7 | 0.1 | 107.2 | 1.1 | 101.2 | 3.3 |
| GSK-3a-b_pS21_S9 | 114.9 | 3.4 | 97.0 | 3.6 | 98.8 | 3.3 |
| Gys | 96.1 | 2.3 | 111.6 | 2.1 | 107.0 | 3.6 |
| Gys_pS641 | 96.7 | 2.6 | 105.2 | 9.0 | 106.2 | 4.4 |
| H2AX_pS140 | 98.3 | 2.5 | 117.7 | 11.1 | 101.9 | 0.8 |
| HER2 | 97.8 | 3.2 | 106.9 | 0.7 | 91.7 | 0.1 |
| HER2_pY1248 | 104.9 | 6.9 | 97.5 | 7.5 | 101.5 | 3.1 |
| HER3 | 100.5 | 0.8 | 102.8 | 1.3 | 98.5 | 0.3 |
| HER3_pY1289 | 99.4 | 0.8 | 96.9 | 3.1 | 98.2 | 2.7 |
| Heregulin | 96.1 | 5.5 | 113.3 | 9.9 | 109.2 | 4.5 |
| HES1 | 96.1 | 4.1 | 92.3 | 2.6 | 97.2 | 0.0 |
| Hexokinase-II | 98.8 | 1.7 | 99.4 | 1.9 | 98.0 | 0.9 |
| Hif-l-alpha | 104.7 | 6.6 | 130.8 | 0.1 | 116.1 | 16.4 |
| Histone-H3 | 96.1 | 5.5 | 131.4 | 1.0 | 123.3 | 20.0 |
| HLA-DR-DP-DQ-DX | 100.0 | 0.0 | 101.7 | 2.4 | 98.9 | 6.0 |
| HSP27 | 98.3 | 2.3 | 96.5 | 2.1 | 101.4 | 4.4 |
| HSP27_pS82 | 96.2 | 5.4 | 95.4 | 1.8 | 114.2 | 2.3 |
| HSP70 | 101.3 | 1.8 | 105.2 | 0.2 | 103.2 | 0.1 |
| IGF1R_pY1135_Y1136 | 99.9 | 3.0 | 97.2 | 4.2 | 98.7 | 3.1 |
| IGFBP2 | 102.3 | 12.3 | 89.0 | 2.0 | 90.9 | 4.5 |
| IGFRb | 93.2 | 1.4 | 98.5 | 3.5 | 99.7 | 2.1 |
| INPP4b | 99.3 | 1.0 | 98.0 | 1.4 | 99.6 | 1.4 |
| IR-b | 93.3 | 1.1 | 111.4 | 5.8 | 110.0 | 7.7 |
| IRF-1 | 103.7 | 5.2 | 111.6 | 1.7 | 104.1 | 0.7 |
| IRS1 | 94.8 | 1.1 | 101.4 | 1.1 | 96.1 | 7.7 |
| Jagged1 | 100.0 | 0.1 | 111.0 | 4.9 | 107.3 | 3.9 |
| Jak2 | 100.6 | 3.9 | 107.2 | 0.3 | 104.3 | 7.7 |
| JNK2 | 99.4 | 0.9 | 103.8 | 7.1 | 111.4 | 9.7 |
| JNK_pT183_Y185 | 106.2 | 5.4 | 98.5 | 5.3 | 99.7 | 4.5 |
| LC3A-B | 114.8 | 21.0 | 106.5 | 9.0 | 102.5 | 0.6 |
| Lck | 99.2 | 1.1 | 96.8 | 3.9 | 101.9 | 0.0 |
| LDHA | 95.0 | 3.7 | 123.8 | 11.9 | 121.2 | 23.5 |
| LRP6_pS1490 | 98.3 | 2.4 | 128.4 | 13.3 | 109.2 | 6.6 |
| MAPK_pT202-Y204 | 97.9 | 0.1 | 93.7 | 0.6 | 98.0 | 1.5 |
| Mcl-1 | 105.2 | 7.3 | 89.4 | 6.6 | 101.9 | 2.0 |
| MCT4 | 97.1 | 0.5 | 126.7 | 16.3 | 119.5 | 21.2 |
| MDM2_pS166 | 100.6 | 0.8 | 94.8 | 1.0 | 92.3 | 1.3 |
| MEK1 | 98.6 | 1.9 | 106.4 | 4.0 | 110.2 | 8.0 |
| MEK1_p_S217-S221 | 95.9 | 3.0 | 95.0 | 0.4 | 98.0 | 0.9 |
| MERIT40_pS29 | 103.3 | 4.0 | 92.3 | 2.6 | 91.1 | 3.4 |
| Merlin | 97.7 | 2.8 | 94.1 | 0.1 | 99.3 | 3.2 |
| MIF | 96.1 | 5.5 | 107.4 | 12.9 | 118.6 | 15.0 |
| MIG6 | 96.1 | 5.5 | 106.7 | 5.3 | 133.9 | 35.5 |
| MMP-14 | 98.4 | 2.2 | 120.9 | 28.2 | 139.1 | 48.8 |
| MMP14-[EP1264Y] | 96.1 | 5.5 | 137.5 | 23.3 | 138.0 | 36.8 |
| MMP2 | 104.8 | 6.8 | 103.9 | 8.4 | 103.4 | 1.6 |
| Mnk1 | 97.9 | 2.3 | 97.2 | 2.8 | 97.5 | 2.3 |
| MSH6 | 101.0 | 1.4 | 97.8 | 0.7 | 99.2 | 1.2 |
| MSI2 | 106.4 | 13.5 | 93.5 | 0.9 | 100.7 | 2.7 |
| mTOR | 94.9 | 0.1 | 98.9 | 0.5 | 92.1 | 6.5 |
| mTOR_pS2448 | 111.4 | 4.4 | 92.3 | 2.6 | 88.3 | 1.1 |
| Myosin-11 | 96.1 | 5.5 | 101.0 | 7.4 | 100.9 | 4.7 |
| Myosin-IIa_pS1943 | 86.7 | 1.6 | 113.7 | 3.7 | 108.6 | 5.7 |
| Myt1 | 102.1 | 3.0 | 93.2 | 1.3 | 94.5 | 0.8 |
| N-Cadherin | 103.5 | 4.9 | 104.0 | 8.5 | 107.8 | 4.7 |
| N-Ras | 100.2 | 0.1 | 98.6 | 0.5 | 93.7 | 2.1 |
| NAPSIN-A | 99.5 | 0.6 | 99.4 | 0.5 | 99.4 | 1.1 |
| NDRG1_pT346 | 112.3 | 17.4 | 118.1 | 17.2 | 103.5 | 1.5 |
| NDUFB4 | 102.9 | 2.8 | 100.5 | 5.1 | 96.3 | 0.3 |
| NF-kB-p65_pS536 | 102.4 | 3.3 | 108.3 | 1.0 | 105.0 | 0.6 |
| Notch1 | 98.2 | 2.6 | 105.8 | 0.2 | 96.2 | 6.3 |
| Notch3 | 93.1 | 1.3 | 112.1 | 4.7 | 105.9 | 3.5 |
| Oct-4 | 103.7 | 0.5 | 102.3 | 4.0 | 100.5 | 5.7 |
| P-Cadherin | 99.8 | 0.3 | 108.3 | 9.8 | 104.3 | 0.4 |
| p21 | 98.4 | 8.6 | 98.7 | 4.1 | 100.5 | 5.7 |
| p27-Kip-1 | 100.6 | 0.8 | 101.1 | 3.6 | 96.7 | 0.9 |
| p27_pT198 | 112.9 | 6.7 | 100.1 | 0.1 | 96.4 | 0.2 |
| p38-MAPK | 95.3 | 2.6 | 93.4 | 1.0 | 94.3 | 3.2 |
| p38_pT180_Y182 | 124.3 | 3.8 | 93.4 | 4.2 | 100.6 | 5.5 |
| p44-42-MAPK | 99.8 | 0.3 | 112.7 | 1.2 | 108.2 | 5.2 |
| p53 | 102.2 | 3.1 | 113.6 | 2.0 | 93.7 | 4.4 |
| p70-S6K1 | 96.8 | 2.9 | 109.6 | 5.1 | 111.0 | 9.2 |
| p70-S6K_pT389 | 110.1 | 3.6 | 92.3 | 2.6 | 95.5 | 4.3 |
| p90RSK_pT573 | 114.9 | 2.5 | 98.5 | 5.2 | 98.9 | 3.4 |
| PAI-1 | 94.4 | 7.5 | 106.1 | 15.6 | 106.1 | 2.1 |
| PAICS | 96.1 | 1.5 | 98.1 | 1.2 | 98.7 | 8.8 |
| PAK1 | 96.5 | 3.2 | 106.0 | 0.1 | 107.0 | 3.5 |
| PAK4 | 95.7 | 10.6 | 95.1 | 0.4 | 95.5 | 2.1 |
| PAR | 72.3 | 6.1 | 158.2 | 5.4 | 135.9 | 15.3 |
| PARP | 100.2 | 0.2 | 104.5 | 6.5 | 98.0 | 2.4 |
| Paxillin | 93.1 | 0.7 | 91.8 | 8.0 | 105.5 | 1.3 |
| PCNA | 101.8 | 2.5 | 97.5 | 6.7 | 88.7 | 1.8 |
| PD-1 | 99.1 | 1.3 | 98.8 | 2.7 | 107.0 | 3.4 |
| PD-L1 | 99.3 | 1.0 | 95.8 | 1.5 | 92.6 | 5.3 |
| Pdcd4 | 96.2 | 5.4 | 120.2 | 9.6 | 117.5 | 18.4 |
| PDGFR-b | 96.1 | 5.5 | 124.5 | 24.1 | 138.5 | 35.4 |
| PDHK1 | 101.4 | 2.0 | 102.2 | 3.6 | 102.5 | 2.9 |
| PDK1 | 102.7 | 3.9 | 99.6 | 1.5 | 99.8 | 2.6 |
| PDK1_pS241 | 103.8 | 5.8 | 103.2 | 0.4 | 97.4 | 1.3 |
| PEA-15 | 98.5 | 1.8 | 97.9 | 0.3 | 98.7 | 1.0 |
| PEA-15_pS116 | 110.2 | 6.1 | 98.0 | 3.8 | 97.0 | 0.7 |
| PI3K-p110-a | 99.1 | 1.2 | 107.4 | 4.5 | 103.7 | 4.7 |
| PI3K-p110-b | 100.2 | 0.2 | 94.8 | 0.1 | 98.5 | 3.0 |
| PI3K-p85 | 101.7 | 5.6 | 100.1 | 5.2 | 100.0 | 4.0 |
| PKA-a | 96.1 | 5.5 | 105.1 | 10.5 | 99.6 | 18.7 |
| PKC-b-II_pS660 | 102.7 | 0.4 | 93.7 | 0.6 | 98.6 | 3.0 |
| PKC-delta_pS664 | 102.7 | 3.8 | 98.8 | 3.7 | 100.9 | 0.5 |
| PKCa | 100.7 | 1.1 | 113.3 | 3.9 | 110.1 | 7.9 |
| PKM2 | 99.1 | 0.8 | 105.8 | 2.2 | 106.2 | 3.1 |
| PLC-gamma2_pY759 | 106.1 | 8.7 | 97.9 | 2.9 | 97.2 | 0.6 |
| PLK1 | 99.7 | 0.4 | 95.0 | 1.4 | 82.1 | 3.8 |
| PMS2 | 98.3 | 1.5 | 96.4 | 3.0 | 98.6 | 0.7 |
| Porin | 98.9 | 1.5 | 97.8 | 1.8 | 95.2 | 1.2 |
| PR | 100.1 | 0.1 | 96.8 | 2.2 | 100.3 | 5.4 |
| PRAS40 | 103.2 | 2.5 | 104.0 | 3.8 | 98.3 | 2.5 |
| PRAS40_pT246 | 114.9 | 0.7 | 92.3 | 2.6 | 90.7 | 2.2 |
| PREX1 | 101.0 | 1.4 | 102.5 | 3.6 | 105.2 | 0.9 |
| PTEN | 98.6 | 0.2 | 124.6 | 1.6 | 108.6 | 6.8 |
| Rab11 | 103.0 | 4.3 | 102.8 | 4.1 | 101.7 | 3.4 |
| Rab25 | 100.7 | 1.0 | 100.2 | 0.4 | 97.8 | 0.5 |
| Rad50 | 100.8 | 1.2 | 103.9 | 0.3 | 104.7 | 0.2 |
| Rad51 | 102.4 | 3.4 | 89.6 | 6.4 | 102.1 | 2.8 |
| Raptor | 99.7 | 1.3 | 103.3 | 0.4 | 98.9 | 2.2 |
| Rb | 109.0 | 11.0 | 106.3 | 14.6 | 93.9 | 0.7 |
| RBM15 | 102.3 | 3.2 | 105.1 | 3.8 | 103.9 | 0.9 |
| Rb_pS807_S811 | 115.0 | 4.7 | 92.3 | 2.6 | 84.3 | 3.5 |
| Rheb | 100.6 | 4.1 | 103.3 | 3.5 | 91.7 | 6.8 |
| Rictor | 99.5 | 0.8 | 126.9 | 13.9 | 111.8 | 10.2 |
| Rictor_pT1135 | 119.5 | 3.5 | 92.3 | 2.6 | 90.0 | 3.5 |
| RIP | 102.4 | 3.3 | 105.6 | 5.6 | 103.7 | 0.8 |
| RPA32 | 99.1 | 0.3 | 98.4 | 2.1 | 96.8 | 4.4 |
| RPA32_pS4-S8 | 93.8 | 4.7 | 102.8 | 11.2 | 93.9 | 17.7 |
| RSK | 96.9 | 1.4 | 99.0 | 5.0 | 104.4 | 3.7 |
| S6 | 104.4 | 6.2 | 112.2 | 11.4 | 102.7 | 13.8 |
| S6_pS235_S236 | 215.8 | 23.6 | 57.3 | 6.2 | 50.9 | 7.9 |
| S6_pS240_S244 | 208.1 | 26.6 | 51.9 | 6.9 | 49.1 | 8.8 |
| SCD | 97.2 | 0.8 | 94.3 | 0.3 | 98.9 | 0.3 |
| SDHA | 103.0 | 4.3 | 102.0 | 0.5 | 103.8 | 1.1 |
| SF2 | 107.4 | 0.5 | 105.3 | 0.3 | 95.8 | 3.0 |
| Shc_pY317 | 98.6 | 1.1 | 100.3 | 5.2 | 104.3 | 3.2 |
| SHP-2_pY542 | 98.1 | 0.4 | 96.4 | 3.2 | 97.2 | 2.8 |
| SLC1A5 | 99.7 | 0.2 | 100.8 | 5.7 | 111.6 | 9.9 |
| Slfn11 | 100.0 | 3.4 | 96.5 | 3.4 | 95.9 | 3.8 |
| Smac | 96.1 | 6.6 | 100.9 | 0.8 | 99.9 | 6.5 |
| Smad1 | 100.7 | 1.0 | 96.6 | 2.6 | 97.8 | 1.6 |
| Smad3 | 99.3 | 0.4 | 110.8 | 3.1 | 102.3 | 3.1 |
| Smad4 | 104.8 | 5.5 | 100.2 | 2.2 | 95.2 | 1.8 |
| Snail | 101.9 | 2.7 | 107.4 | 8.6 | 101.3 | 4.7 |
| SOD1- | 102.1 | 4.8 | 101.4 | 1.8 | 97.7 | 1.7 |
| SOD2 | 96.1 | 5.5 | 103.5 | 2.5 | 106.0 | 3.7 |
| Sox2 | 102.0 | 2.6 | 105.3 | 2.6 | 100.5 | 5.7 |
| Src | 99.0 | 1.4 | 103.5 | 2.1 | 103.9 | 1.0 |
| Src_pY419 | 100.4 | 0.6 | 98.9 | 5.1 | 99.6 | 0.4 |
| Src_pY527 | 99.4 | 0.8 | 91.0 | 0.1 | 93.7 | 5.7 |
| Stat3 | 95.6 | 1.7 | 99.2 | 1.3 | 92.9 | 6.9 |
| Stat3_pY705 | 105.1 | 6.6 | 119.9 | 10.8 | 100.5 | 5.7 |
| Stat5a | 99.8 | 2.2 | 111.4 | 1.0 | 108.2 | 7.6 |
| Stathmin-1 | 100.9 | 1.3 | 101.8 | 7.5 | 99.3 | 3.3 |
| STING | 96.1 | 5.5 | 136.3 | 14.0 | 135.4 | 23.0 |
| Syk | 98.9 | 1.6 | 92.8 | 1.8 | 95.8 | 0.5 |
| Tau | 100.8 | 1.2 | 101.7 | 0.6 | 102.9 | 2.3 |
| TAZ | 121.6 | 14.2 | 89.0 | 7.2 | 100.5 | 5.6 |
| TFAM | 105.8 | 8.2 | 107.8 | 2.4 | 101.4 | 2.7 |
| TFRC | 108.6 | 6.8 | 99.8 | 2.0 | 96.3 | 0.3 |
| TIGAR | 99.3 | 0.9 | 96.8 | 5.1 | 100.5 | 1.9 |
| Transglutaminase | 103.3 | 4.7 | 107.8 | 13.4 | 93.5 | 3.2 |
| TRIM25 | 91.9 | 0.4 | 108.0 | 7.4 | 99.8 | 4.5 |
| TSC1 | 96.9 | 2.1 | 106.5 | 0.1 | 102.5 | 5.7 |
| TTF1 | 102.0 | 2.8 | 102.7 | 4.1 | 95.4 | 1.8 |
| Tuberin | 99.6 | 0.6 | 106.3 | 1.3 | 101.7 | 2.9 |
| Tuberin_pT1462 | 109.1 | 13.3 | 93.5 | 1.1 | 93.6 | 2.2 |
| TUFM | 103.4 | 0.8 | 96.9 | 2.7 | 98.4 | 2.7 |
| TWIST | 99.2 | 1.1 | 96.9 | 0.2 | 97.7 | 1.6 |
| Tyro3 | 101.9 | 1.5 | 98.8 | 5.5 | 98.5 | 2.8 |
| U-Histone-H2B | 86.4 | 9.5 | 108.7 | 0.8 | 113.8 | 13.1 |
| UBAC1 | 95.7 | 0.8 | 100.4 | 1.3 | 99.7 | 3.0 |
| UGT1A | 95.5 | 1.1 | 94.3 | 0.2 | 98.6 | 2.8 |
| ULK1_pS757 | 101.7 | 2.4 | 100.9 | 0.0 | 102.1 | 0.5 |
| VASP | 83.8 | 8.1 | 108.6 | 7.7 | 100.5 | 5.7 |
| VEGFR-2 | 93.7 | 1.5 | 93.7 | 0.5 | 93.5 | 4.2 |
| VHL-EPPK1 | 96.1 | 5.5 | 90.7 | 1.1 | 88.7 | 4.5 |
| Wee1 | 110.5 | 3.4 | 92.3 | 2.6 | 95.3 | 4.7 |
| Wee1_pS642 | 109.3 | 4.6 | 94.7 | 3.3 | 95.2 | 4.6 |
| WIPI1 | 328.3 | 322.8 | 112.5 | 16.1 | 102.3 | 3.1 |
| WIPI2 | 102.0 | 3.5 | 108.8 | 2.8 | 106.2 | 2.4 |
| XBP-1 | 101.1 | 1.6 | 92.8 | 1.9 | 89.5 | 0.2 |
| XPA | 96.9 | 4.3 | 96.4 | 3.3 | 83.5 | 8.5 |
| XPF | 101.1 | 4.0 | 109.0 | 1.5 | 100.5 | 3.3 |
| XRCC1 | 99.9 | 1.0 | 104.0 | 5.4 | 102.3 | 2.7 |
| YAP | 100.4 | 1.4 | 95.4 | 1.9 | 93.7 | 3.6 |
| YAP_pS127 | 98.8 | 1.8 | 106.2 | 0.4 | 103.1 | 2.1 |
| YB1_pS102 | 112.1 | 0.4 | 92.3 | 2.6 | 82.7 | 3.6 |
| ZAP-70 | 100.4 | 0.6 | 92.5 | 2.2 | 95.1 | 2.8 |

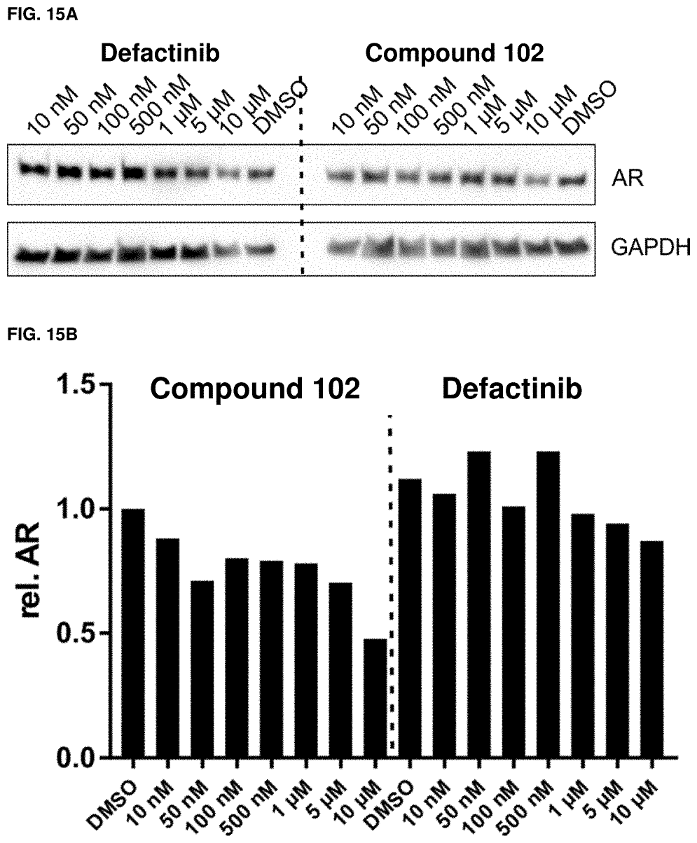

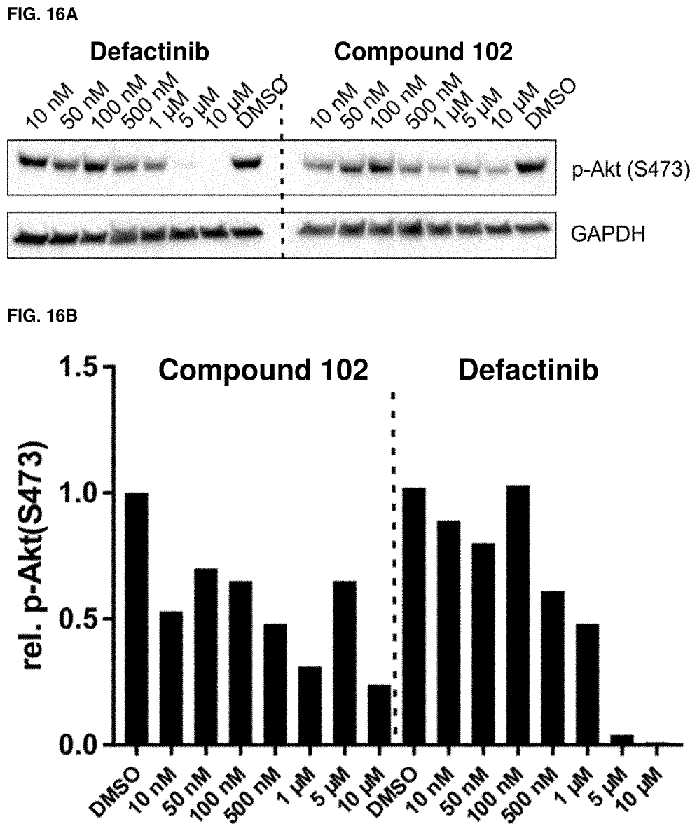

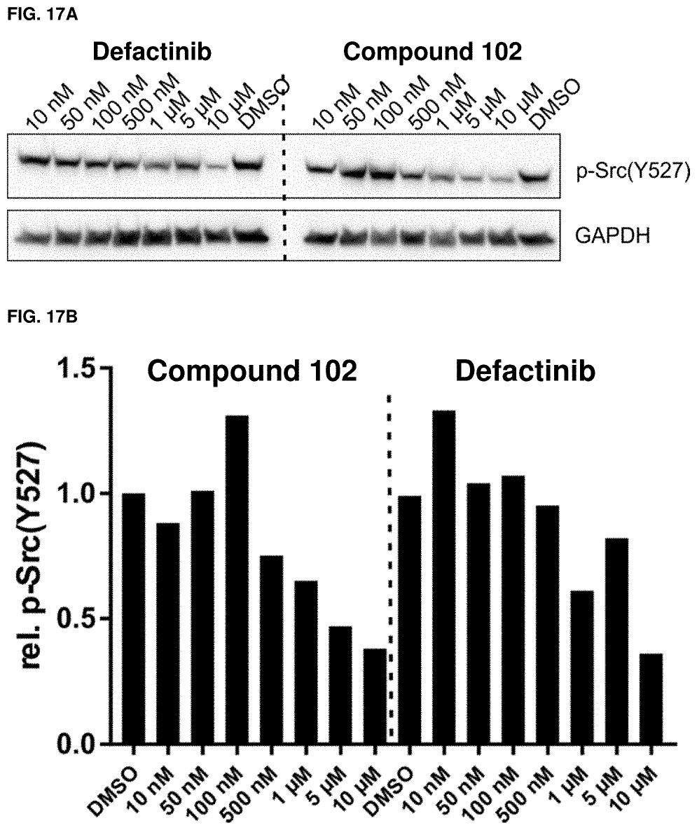

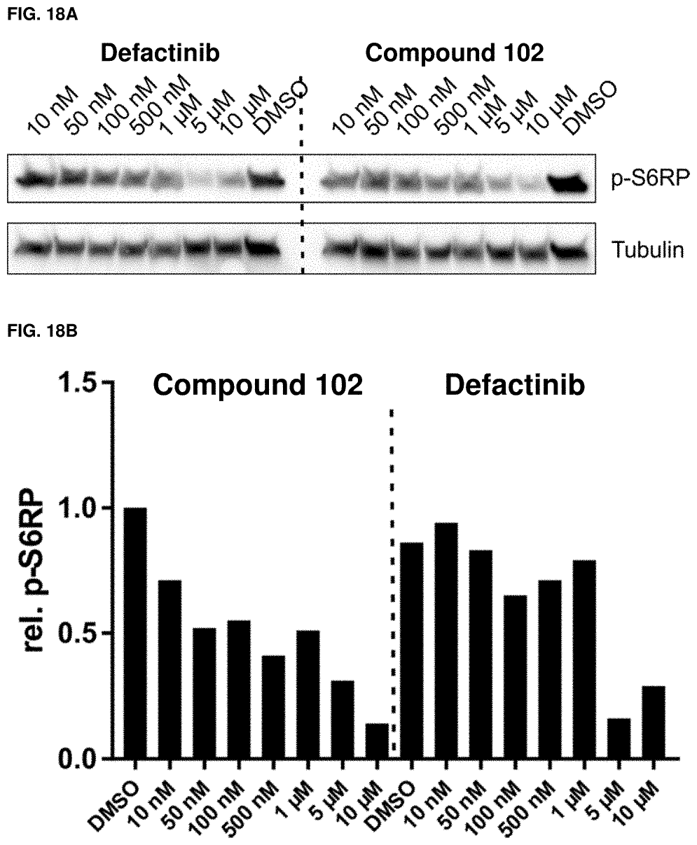

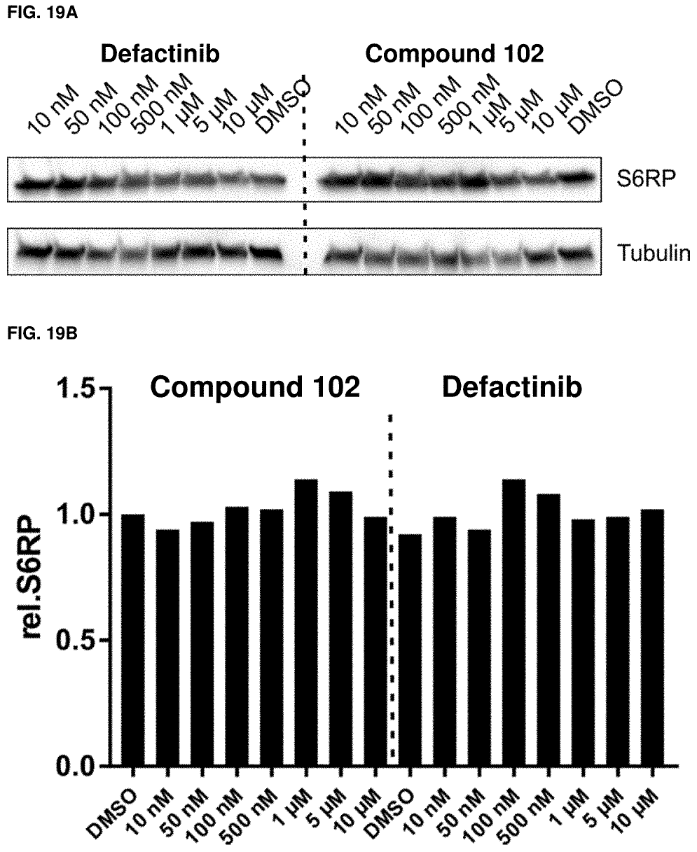

RPPA results confirmed Fak degradation and reduced levels of p-Fak in exemplary compound 102 treated cells as well as reduced p-Fak levels after defactinib treatment. As discussed below, the RPPA results suggest several pathways and/or scaffolding event that may be responsible for the effects on migration and invasion. Changes in protein levels observed by RPPA were validated by western blotting from cell lysates after incubation of MDA-MB-231 cell with varying concentrations of exemplary compound 102 and defactinib in the presence of serum (FIGS. 15A, 15B, 16A, 16B, 17A, 17B, 18A, 18B, 19A, and 19B). The most surprising effect was observed for the androgen receptor (AR) (FIGS. 15A and 15B). It has been previously shown that extranuclear AR is involved in cell migration and forms a multiprotein complex comprised of filamin A/β-1 integrin/Fak/AR in NIH3T3 fibroblasts that facilitates Fak activation. Based on the obtained RPPA data and verified from MDA-MB-231 cell lysates, a reduction of AR levels after exemplary compound 102 treatment was observed by western blotting (FIGS. 15A and 15B). As no similar effect on AR in defactinib-treated cells is observed, this suggests a specific involvement of extranuclear AR in Fak scaffold signaling and Fak mediated cell motility. Besides the changes in AR, reduced levels of p-Akt(S473) and p-Src(Y527) can be detected as well (FIGS. 16A, 16B, 17A, and 17B). While p-Akt was already characterized previously in PC3 cells (FIGS. 8A-8D), differences in p-Src(Y527) may arise from a disruption of the Fak-Src complex upon exemplary compound 102 mediated Fak degradation. The effect of defactinib on p-Src(Y527) at high concentrations might be based on off-target Src binding (KINOMEScan data not shown). Additionally, reduced phosphorylation of the S6 ribosomal protein (S6RP) in exemplary compound 102 treated cells can be observed while total S6RP levels remained unchanged (FIGS. 18A, 18B, 19A, and 19B). Phosphorylation of S6RP occurs via the Src-Fak-PI3K pathway and p-S6RP is required for the initiation of translation in response to cell growth and proliferation.

Discussion.

The data of the present disclosure highlight the advantages of protein degradation over protein inhibition for proteins like Fak that act via kinase-dependent, as well as kinase-independent, signaling. While defactinib has no effect at nanomolar concentrations on downstream signaling, cell migration and invasion, exemplary compound 102 significantly reduces signaling as well as the ability of TNBC cells to migrate and invade. These results exemplify the differential biology that results from different MOAs based on the selected modality.

Within the past decade, medicinal chemistry has increasingly faced the challenges of expanding the druggable space as more promising therapeutic targets are proposed that are yet out of reach of the traditional approaches. In this context, PROTACs are taking a leading role in advancing the druggable space as they facilitate effective degradation of a protein target using small molecule like chemical entities. PROTACs not only allow the targeting of novel proteins that thus far out of reach, but they also allow targeting of additional functions of already established drug targets due to a different MOA. To the inventors knowledge, exemplary compound 102 is the first degrader that outperforms an optimized kinase inhibitor due to its orthogonal MOA, allowing the exemplary compound to modulate effects that are unobtainable with an inhibitor.

Pharmacokinetic Data of Exemplary Compounds.

PK-data was obtained and is summarized in Table 5 below.

TABLE 5
Summary of PK-data for Exemplary Compounds
100, 101, 102, 103, and 108

| | | | AUClast | AUClast | |
|---|---|---|---|---|---|
| Compound | T ½ (h) | T ½ (h) | (h * ng/mL) | (h * ng/mL) | PPB |
| Number | IV | IP | IV | IP | (%) |
| 108 | 0.058 | 1.10 | 3.80 | 208 | — |
| 103 | 2.44 | 1.87 | 747 | 10364 | 99.78 |
| 100 | 1.95 | 2.15 | 175 | 5868 | 99.20 |
| 101 | 1.26 | 1.22 | 268 | 6505 | 99.35 |
| 102 | 0.905 | 1.46 | 264 | 6264 | 99.34 |

In vivo degradation in black6 mice was observed and is summarized in table 6 below.

TABLE 6
In vivo Fak degradation

| Compound | | | Tissue % degradation | | |
|---|---|---|---|---|---|
| Name | C mg/kg | IP every x h | spleen | lung | thymus |
| 103 | 95 | 24 | 45 | 60 | 75 |

Pharmacokinetics of Intraperitoneal or Intravenous Injection of Exemplary Compounds 103, 107, 100, 101, and 102.

The pharmacokinetics of exemplary compounds 103, 107, 100, 101, and 102 was examined for intraperitoneal and intravenous administration. The desired serial concentrations of working solutions were achieved by diluting stock solution (1 mg/mL in DMSO) of analyte with methanol. In particular, 20 μL of working solutions (1, 2, 5, 10, 50, 100, 500, 1000, 2000 ng/mL) were added to 20 μL of the blank CD1 mice plasma to achieve calibration standards of 1-2000 ng/mL (1, 2, 5, 10, 50, 100, 500, 1000, 2000 ng/mL) in a total volume of 40 μL. Dose of IV and IP were diluted by 1000 fold and 10000 with methanol, 20 μL of the diluted dose solutions were added to 20 μL of the blank CD1 mice plasma to achieve dose samples. Next, 40 μL standards and 40 μL dose samples were added to 20 μL of IS (100 ng/mL) and 200 μL of acetonitrile for precipitating protein respectively. Then the samples were vortexed for 30 seconds. After centrifugation at 4 degree Celsius, 4000 rpm for 15 minutes, the supernatant was diluted 3 times with water. 10 μL of the diluted supernatant was injected into the LC/MS/MS system for quantitative analysis.

Exemplary compounds 103, 107, 100, 101, and 102 were administered intravenously (5 second injection of 10% HP-b-CD and 40 mM sodium acetate and 40 mM NaCl in water, pH4.5) or intraperitoneally (5% EtOH and 5% Solutol HS15 in D5W(ESD-2)) to 6-8 week old male CD1 mice (3 mice per treatment group for a total of 12 mice) at the time points indicated below in Table 7. The mice had free access to food and water.

TABLE 7
Pharmacokinetics study design for Exemplary Compounds
103, 107, 100, 101, and 102

| | | | Dosing | |
|---|---|---|---|---|
| | | Dosing | Concen- | Dosing |
| Route of | | Level | tration | Volume |
| Administration | Time Points | (mg/kg) | (mg/mL) | (mL/kg) |
| IV | 0.033 h, 0.083 h, | 1.0 | 0.2 | 5 |
| | 0.25 h, 0.5 h, 1 h, | | | |
| | 2 h, 4 h, 8 h, 24 h | | | |
| IP | 0.25 h, 0.5 h, 1 h, 2 h, | 10 | 1.0 | 10 |
| | 4 h, 8 h, 24 h | | | |

Dorsal metatarsal vein samples (30 μL), except that the final sample was acquired via a heart puncture, were taken from the mice at the same the administration time points as shown in Table 7.

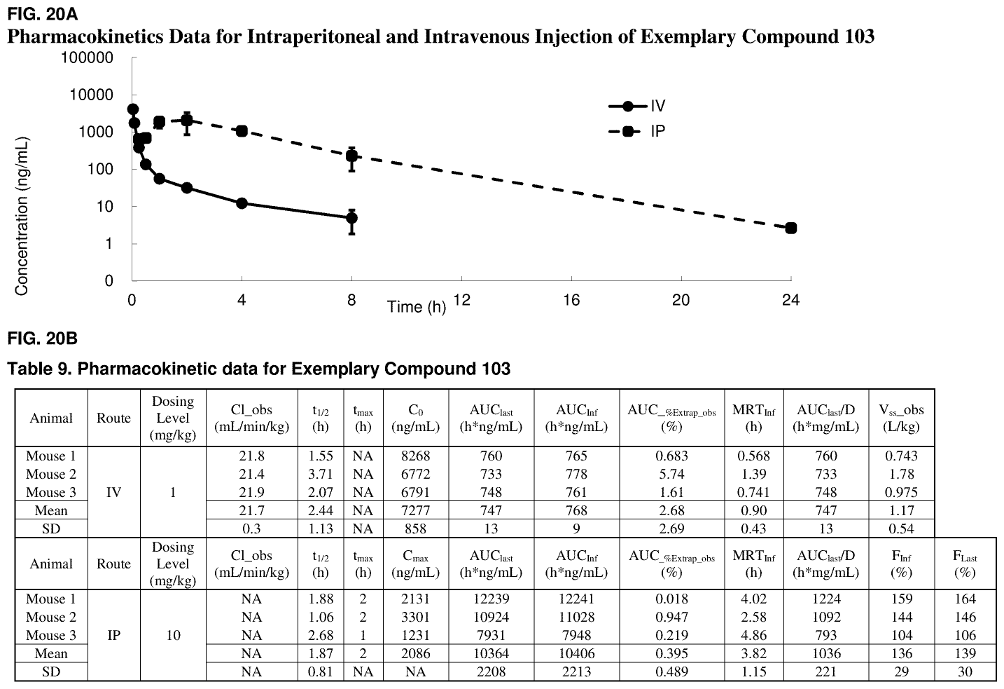

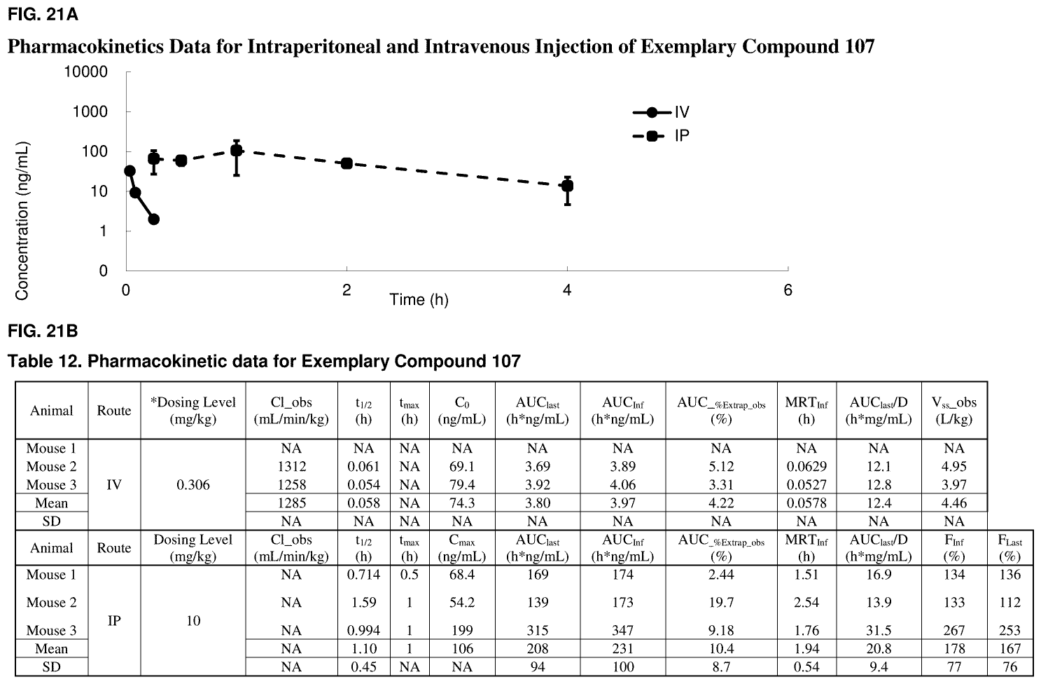

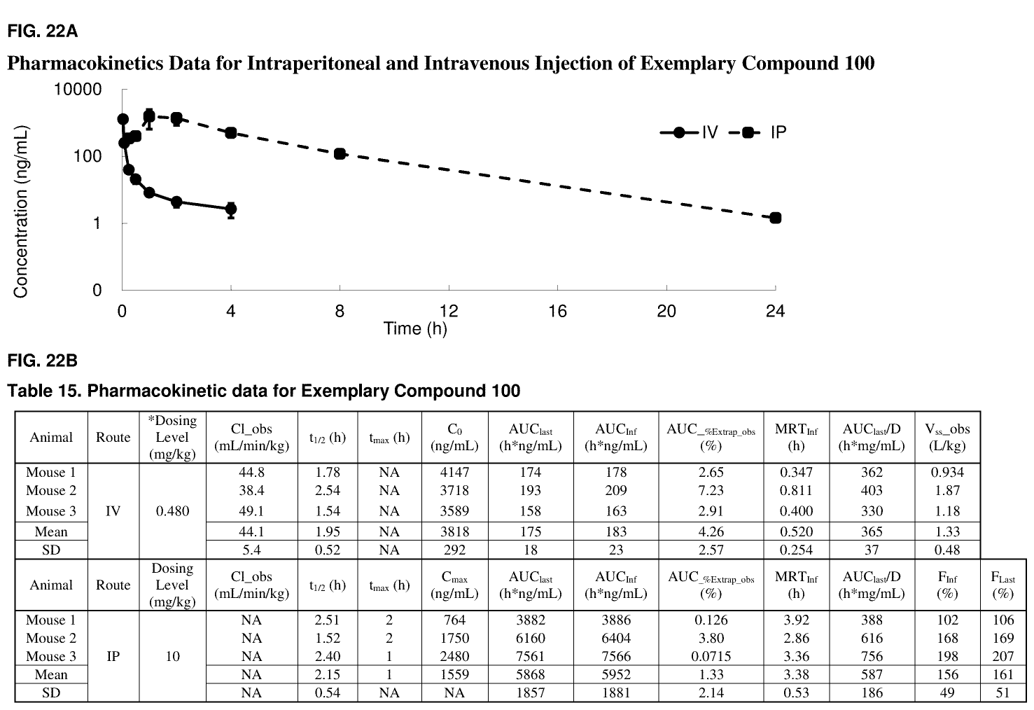

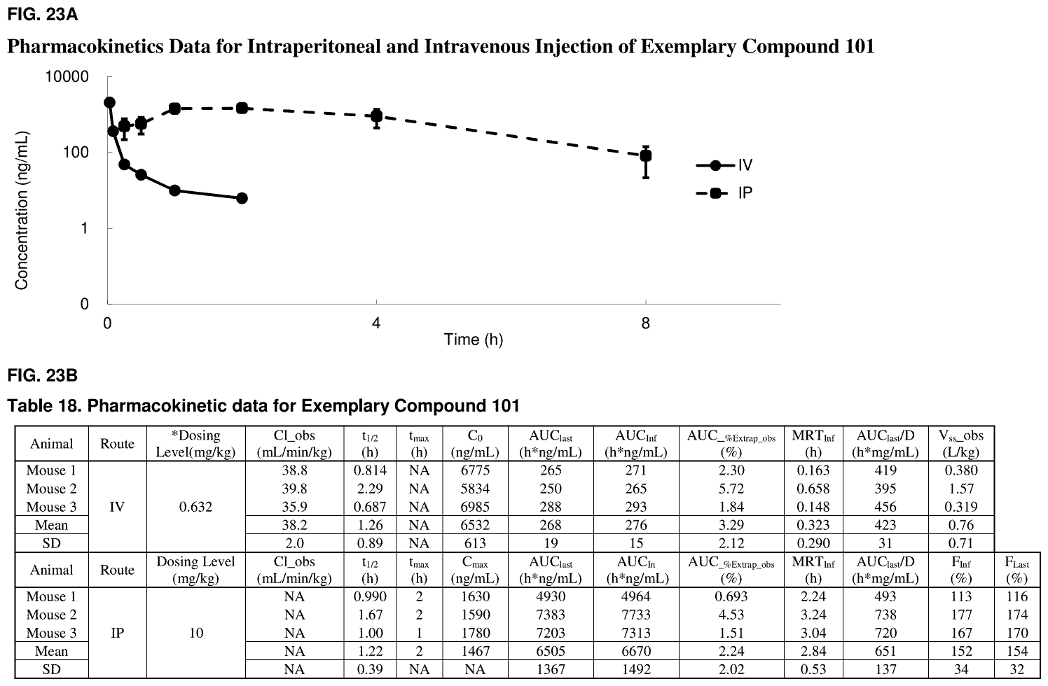

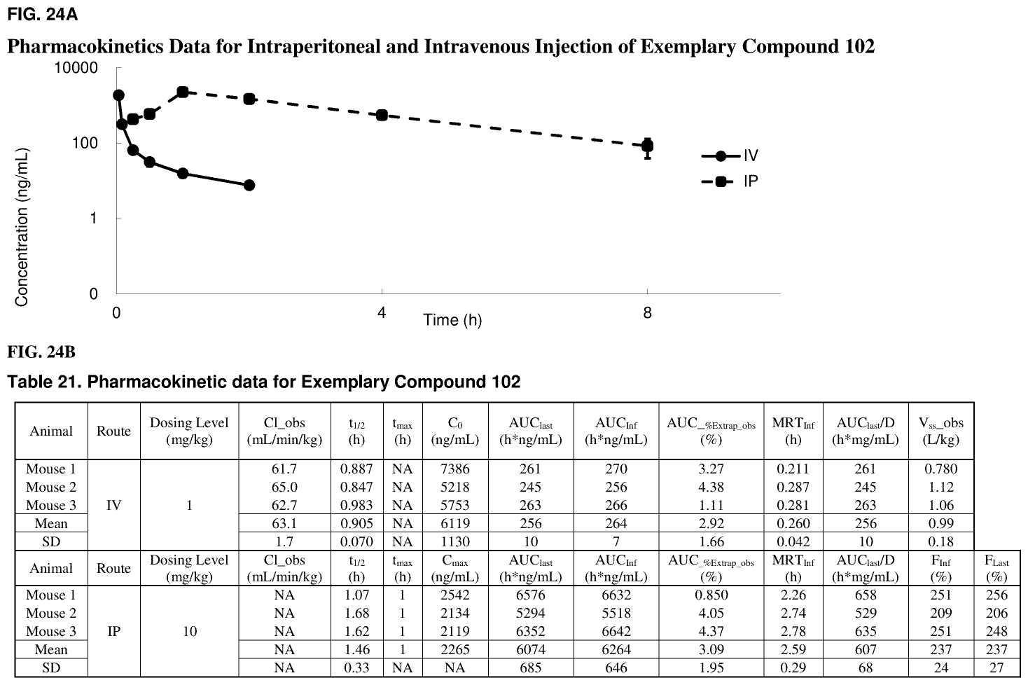

The bioanalytical assay using an internal standard was performed on the samples as shown in Table 8 below. The data for exemplary compound 103 is shown below in FIG. 20A and Tables 9 (FIG. 20B), 10 and 11. The data for exemplary compound 107 is shown in FIG. 21A and Tables 12 (FIG. 21B), 13 and 14. The data for exemplary compound 100 is shown in FIG. 22A and Tables 15 (FIG. 22B), 16 and 17. The data for exemplary compound 101 is shown in FIG. 23A and Table 18 (FIG. 23B), 19 and 20. The data for exemplary compound 102 is shown in FIG. 24A and Table 21 (FIG. 24B), 22 and 23.

TABLE 8
Bioanalysis of the pharmacokinetic examination of
Example 1 and Example 2

| | | |
|---|---|---|
| Bioanalytical | HPLC | Instrument: SHIMADZU (LC-30A D; |
| Assay: | | DGU-20A5R; CBM-20A; SIL-30AC; |
| | | CTO-30A); Rack Changer II |
| | MS | LCMS-8050 instrument |
| | Column | Phenomenex Kinetex 5u C18 |
| | | (50 * 2.1 mm) |
| | Mobile Phase | 95% Water (0.1% Formic Acid) and 95% |
| | | Acetonitrile(0.1% Formic Acid) |
| | Quantification | Internal Standard Method |

TABLE 10
Pharmacokinetic data for intravenously
injected Exemplary Compound 103

| Time | Conc. (ng/mL) | | | Mean | SD |
|---|---|---|---|---|---|
| (h) | Mouse 1 | Mouse 2 | Mouse 3 | (ng/mL) | (ng/mL) |
| 0.033 | 4372 | 3938 | 4025 | 4112 | 230 |
| 0.083 | 1665 | 1732 | 1822 | 1740 | 78 |
| 0.25 | 376 | 382 | 395 | 384 | 10 |
| 0.5 | 140 | 132 | 135 | 136 | 4 |
| 1 | 52.1 | 58.8 | 57.5 | 56.2 | 3.5 |
| 2 | 36.1 | 27.7 | 32.1 | 32.0 | 4.2 |
| 4 | 10.8 | 12.9 | 13.0 | 12.3 | 1.2 |
| 8 | 2.33 | 8.35 | 4.11 | 4.93 | 3.09 |
| 24 | BLOQ | BLOQ | BLOQ | NA | NA |

TABLE 11
Pharmacokinetic data for intraperitoneally
injected Exemplary Compound 103

| Time | Conc. (ng/mL) | | | Mean | SD |
|---|---|---|---|---|---|
| (h) | Mouse 1 | Mouse 2 | Mouse 3 | (ng/mL) | (ng/mL) |
| 0.25 | 877 | 494 | 562 | 645 | 204 |
| 0.5 | 842 | 639 | 608 | 696 | 127 |
| 1 | 2006 | 2460 | 1231 | 1899 | 621 |
| 2 | 2131 | 3301 | 826 | 2086 | 1238 |
| 4 | 1262 | 1209 | 768 | 1080 | 271 |
| 8 | 321 | 68.5 | 306 | 232 | 142 |
| 24 | 0.824 | BLOQ | 4.50 | 2.66 | NA |

TABLE 13
Pharmacokinetic data for intravenously
injected Exemplary Compound 107

| Time | Conc. (ng/mL) | | | Mean | SD |
|---|---|---|---|---|---|
| (h) | Mouse 1 | Mouse 2 | Mouse 3 | (ng/mL) | (ng/mL) |
| 0.033 | 31.4 | 31.6 | 34.3 | 32.4 | 1.6 |
| 0.083 | 8.44 | 9.65 | 9.61 | 9.23 | 0.69 |
| 0.25 | BLOQ | 2.25 | 1.73 | 1.99 | NA |
| 0.5 | BLOQ | BLOQ | BLOQ | NA | NA |
| 1 | BLOQ | BLOQ | BLOQ | NA | NA |
| 2 | BLOQ | BLOQ | BLOQ | NA | NA |
| 4 | BLOQ | BLOQ | BLOQ | NA | NA |
| 8 | BLOQ | BLOQ | BLOQ | NA | NA |
| 24 | BLOQ | BLOQ | BLOQ | NA | NA |

TABLE 14
Pharmacokinetic data for intraperitoneally
injected Exemplary Compound 107

| Time | Conc. (ng/mL) | | | Mean | SD |
|---|---|---|---|---|---|
| (h) | Mouse 1 | Mouse 2 | Mouse 3 | (ng/mL) | (ng/mL) |
| 0.25 | 44.8 | 42.6 | 111 | 66 | 39 |
| 0.5 | 68.4 | 41.9 | 69.3 | 59.9 | 15.6 |
| 1 | 64.7 | 54.2 | 199 | 106 | 81 |
| 2 | 53.2 | 38.1 | 59.8 | 50.4 | 11.1 |
| 4 | 4.11 | 14.9 | 22.2 | 13.7 | 9.1 |
| 8 | BLOQ | BLOQ | BLOQ | NA | NA |
| 24 | BLOQ | BLOQ | BLOQ | NA | NA |

TABLE 16
Pharmacokinetic data for intravenously
injected Exemplary Compound 100

| Time | Conc. (ng/mL) | | | Mean | SD |
|---|---|---|---|---|---|
| (h) | Mouse 1 | Mouse 2 | Mouse 3 | (ng/mL) | (ng/mL) |
| 0.033 | 1320 | 1390 | 1150 | 1287 | 123 |
| 0.083 | 233 | 313 | 205 | 250 | 56 |
| 0.25 | 34.7 | 48.2 | 36.4 | 39.8 | 7.4 |
| 0.5 | 17.4 | 27.1 | 18.0 | 20.8 | 5.4 |
| 1 | 6.22 | 9.77 | 8.79 | 8.26 | 1.83 |
| 2 | 3.27 | 5.86 | 3.98 | 4.37 | 1.34 |
| 4 | 1.84 | 4.11 | 2.13 | 2.69 | 1.24 |
| 8 | BLOQ | BLOQ | BLOQ | NA | NA |
| 24 | BLOQ | BLOQ | BLOQ | NA | NA |

TABLE 17
Pharmacokinetic data for intraperitoneally
injected Exemplary Compound 100

| Time | Conc. (ng/mL) | | | Mean | SD |
|---|---|---|---|---|---|
| (h) | Mouse 1 | Mouse 2 | Mouse 3 | (ng/mL) | (ng/mL) |
| 0.25 | 252 | 402 | 374 | 343 | 80 |
| 0.5 | 362 | 467 | 373 | 401 | 58 |
| 1 | 648 | 1550 | 2480 | 1559 | 916 |
| 2 | 764 | 1750 | 1610 | 1375 | 533 |
| 4 | 362 | 625 | 510 | 499 | 132 |
| 8 | 95.4 | 111 | 151 | 119 | 29 |
| 24 | 1.35 | BLOQ | 1.56 | 1.46 | NA |

TABLE 19
Pharmacokinetic data for intravenously
injected Exemplary Compound 101

| Time | Conc. (ng/mL) | | | Mean | SD |
|---|---|---|---|---|---|
| (h) | Mouse 1 | Mouse 2 | Mouse 3 | (ng/mL) | (ng/mL) |
| 0.033 | 2100 | 1800 | 2310 | 2070 | 256 |
| 0.083 | 356 | 303 | 432 | 364 | 65 |
| 0.25 | 50.1 | 47.7 | 47.0 | 48.3 | 1.6 |
| 0.5 | 20.9 | 28.6 | 27.7 | 25.7 | 4.2 |
| 1 | 8.68 | 11.6 | 9.52 | 9.9 | 1.5 |
| 2 | 5.32 | 7.86 | 5.45 | 6.21 | 1.43 |
| 4 | BLOQ | 4.59 | BLOQ | NA | NA |
| 8 | BLOQ | BLOQ | BLOQ | NA | NA |
| 24 | BLOQ | BLOQ | BLOQ | NA | NA |

TABLE 20
Pharmacokinetic data for intraperitoneally
injected Exemplary Compound 101

| Time | Conc. (ng/mL) | | | Mean | SD |
|---|---|---|---|---|---|
| (h) | Mouse 1 | Mouse 2 | Mouse 3 | (ng/mL) | (ng/mL) |
| 0.25 | 800 | 407 | 271 | 493 | 275 |
| 0.5 | 860 | 523 | 335 | 573 | 266 |
| 1 | 1040 | 1520 | 1780 | 1447 | 375 |
| 2 | 1630 | 1590 | 1180 | 1467 | 249 |
| 4 | 378 | 1090 | 1250 | 906 | 464 |
| 8 | 24.1 | 145 | 77.1 | 82 | 61 |
| 24 | BLOQ | BLOQ | BLOQ | NA | NA |

TABLE 22
Pharmacokinetic data for intravenously
injected Exemplary Compound 102

| Time | Conc. (ng/mL) | | | Mean | SD |
|---|---|---|---|---|---|
| (h) | Mouse 1 | Mouse 2 | Mouse 3 | (ng/mL) | (ng/mL) |
| 0.033 | 1919 | 1818 | 1853 | 1863 | 51 |
| 0.083 | 249 | 368 | 333 | 317 | 61 |
| 0.25 | 55.9 | 67.4 | 71.8 | 65.1 | 8.2 |
| 0.5 | 23.7 | 33.2 | 37.6 | 31.5 | 7.1 |
| 1 | 11.8 | 16.7 | 18.4 | 15.6 | 3.4 |
| 2 | 6.90 | 9.20 | 6.75 | 7.62 | 1.38 |
| 4 | BLOQ | BLOQ | 2.09 | NA | NA |
| 8 | BLOQ | BLOQ | BLOQ | NA | NA |
| 24 | BLOQ | BLOQ | BLOQ | NA | NA |

TABLE 23
Pharmacokinetic data for intraperitoneally
injected Exemplary Compound 102

| Time | Conc. (ng/mL) | | | Mean | SD |
|---|---|---|---|---|---|
| (h) | Mouse 1 | Mouse 2 | Mouse 3 | (ng/mL) | (ng/mL) |
| 0.25 | 521 | 432 | 338 | 431 | 92 |
| 0.5 | 674 | 546 | 551 | 591 | 72 |
| 1 | 2542 | 2134 | 2119 | 2265 | 240 |
| 2 | 1689 | 1073 | 1698 | 1487 | 358 |
| 4 | 560 | 529 | 559 | 549 | 17 |
| 8 | 36.4 | 92.3 | 124 | 84 | 44 |
| 24 | BLOQ | BLOQ | BLOQ | NA | NA |

The IV and IP routes of administration were effective at increasing plasma levels of Exemplary Compounds 103, 107, 100, 101, and 102. As can be seen in the data, when an order of magnitude more is adminstered via IP, there is a concomitant increase in Cmax and AUC.

Focal adhesion kinase (Fak/PTK2) represents a promising cancer target as it is involved in tumor growth, invasion and metastasis. In the Examples and results above the use of FAK protein degradation via small molecule bifunctional molecules is shown as an effective approach to impeding tumor cell mobility and metastasis.

The contents of all references, patents, pending patent applications and published patents, cited throughout this application are hereby expressly incorporated by reference.

Those skilled in the art will recognize, or be able to ascertain using no more than routine experimentation, many equivalents to the specific embodiments of the disclosure described herein. Such equivalents are intended to be encompassed by the following claims. It is understood that the detailed examples and embodiments described herein are given by way of example for illustrative purposes only, and are in no way considered to be limiting to the disclosure. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present disclosure will be apparent from the appended claims. Moreover, those skilled in the art will recognize, or be able to ascertain using no more than routine experimentation, many equivalents to the specific embodiments of the disclosure described herein. Such equivalents are intended to be encompassed by the following claims.

### REFERENCES

(1) Lee, B. Y.; Timpson, P.; Horvath, L. G.; Daly, R. J. FAK signaling in human cancer as a target for therapeutics. Pharmacol. Ther. 2015, 146, 132-149.
(2) Sulzmaier, F. J.; Jean, C.; Schlaepfer, D. D. FAK in cancer: mechanistic findings and clinical applications. Nat. Rev. Cancer 2014, 14, 598-610.
(3) Cance, W. G.; Kurenova, E.; Marlowe, T.; Golubovskaya, V. Disrupting the scaffold to improve focal adhesion kinase-targeted cancer therapeutics. Sci. Signal 2013, 6, pe10.
(4) Schaller, M. D. Cellular functions of FAK kinases: insight into molecular mechanisms and novel functions. J. Cell. Sci. 2010, 123, 1007-1013.
(5) Mitra, S. K.; Hanson, D. A.; Schlaepfer, D. D. Focal adhesion kinase: in command and control of cell motility. Nat. Rev. Mol. Cell Biol., 2005, 6, 56-68.
(6) Frame, M. C.; Patel, H.; Serrels, B.; Lietha, D.; Eck, M. J. The PERM domain: organizing the structure and function of FAK. Nat. Rev. Mol. Cell Biol. 2010, 11, 802-814.
(7) Pylayeva, Y.; Gillen, K. M.; Gerald, W.; Beggs, H. E.; Reichardt, L. F.; Giancotti, F. G. Ras- and PDK-dependent breast tumorigenesis in mice and humans requires focal adhesion kinase signaling. J. Clin. Invest. 2009, 119, 252-266.
(8) Ott, G. R.; Cheng, M.; Learn, K. S.; Wagner, J.; Gingrich, D. E.; Lisko, J. G.; Curry, M.; Mesaros, E. F.; Ghose, A. K.; Quail, M. R. et al. Discovery of Clinical Candidate CEP-37440, a Selective Inhibitor of Focal Adhesion Kinase (FAK) and Anaplastic Lymphoma Kinase (ALK). J. Med. Chem. 2016.
(9) Beraud, C.; Dormoy, V.; Danilin, S.; Lindner, V.; Bethry, A.; Hochane, M.; Coquard, C.; Barthelmebs, M.; Jacqmin, D.; Lang, H. et al. Targeting FAK scaffold functions inhibits human renal cell carcinoma growth, Int J Cancer 2015, 137, 1549-1559.
(10) Fan, H.; Zhao, X.; Sun, S.; Luo, M.; Guan, J.-L. Function of focal adhesion kinase scaffolding to mediate endophilin A2 phosphorylation promotes epithelial-mesenchymal transition and mammary cancer stem cell activities in vivo. J. Biol. Chem. 2013, 288, 3322-3333.
(11) Burslem, G. M.; Crews, C. M. Small-Molecule Modulation of Protein Homeostasis. Chem. Rev. 2017.
(12) Cromm, P. M.; Crews, C. M. Targeted Protein Degradation: From Chemical Biology to Drug Discovery. Cell Chem. Biol. 2017, 24, pi 181-1190.
(13) Ottis, P.; Crews, C. M. Proteolysis-Targeting Chimeras: Induced Protein Degradation as a Therapeutic Strategy. ACS Chem. Biol. 2017, 12, 892-898.
(14) Sakamoto, K. M.; Kim, K. B.; Kumagai, A.; Mercuric, F.; Crews, C. M.; Deshaies, R. J. Protacs: chimeric molecules that target proteins to the Skp1-Cullin-F box complex for ubiquitination and degradation. Proc. Natl. Acad. Sci. U.S.A. 2001, 98, 8554-8559.
(15) Schneekloth, J. S., JR; Fonseca, F. N.; Koldobskiy, M.; Mandal, A.; Deshaies, R.;
   Sakamoto, K.; Crews, C. M. Chemical genetic control of protein levels: selective in vivo targeted degradation. J. Am. Chem. Soc. 2004, 126, 3748-3754.
(16) Schneekloth, A. R.; Pucheault, M.; Tae, H. S.; Crews, C. M. Targeted intracellular protein degradation induced by a small molecule: En route to chemical proteomics. Bioorg. Med. Chem. Lett. 2008, 18, 5904-5908.
(17) Itoh, Y.; Ishikawa, M.; Naito, M.; Hashimoto, Y. Protein knockdown using methyl bestatin-ligand hybrid molecules: design and synthesis of inducers of ubiquitination-mediated degradation of cellular retinoic acid-binding proteins. J. Am. Chem. Soc. 2010, 132, 5820-5826.
(18) Itoh, Y.; Ishikawa, M.; Kitaguchi, R.; Sato, S.; Naito, M.; Hashimoto, Y. Development of target protein-selective degradation inducer for protein knockdown. Bioorg. Med. Chem. 2011, 19, 3229-3241.
(19) Bondeson, D. P.; Mares, A.; Smith, I. E. D.; Ko, E.; Campos, S.; Miah, A. H.; Mulholland, K. E.; Routly, N.; Buckley, D. L.; Gustafson, J. L. et al. Catalytic in vivo protein knockdown by small-molecule PROTACs. Nat. Chem. Biol. 2015, 11, 611-617.
(20) Winter, G. E.; Buckley, D. L.; Paulk, J.; Roberts, J. M.; Souza, A.; Dhe-Paganon, S.; Bradner, J. E. Phthalimide conjugation as a strategy for in vivo target protein degradation. Science 2015, 348, 1376-1381.
(21) Lu, J.; Qian, Y.; Altieri, M.; Dong, H.; Wang, J.; Raina, K.; Hines, J.; Winkler, J. D.; Crew, A. P.; Coleman, K. et al. Hijacking the E3 Ubiquitin Ligase Cereblon to Efficiently Target BRIM. Chem. Biol. 2015, 22, 755-763.
(22) Galdeano, C.; Gadd, M. S.; Soares, P.; Scaffidi, S.; van Molle, I.; Birced, I.; Hewitt, S.; Dias, D. M.; Ciulli, A. Structure-guided design and optimization of small molecules targeting the protein-protein interaction between the von Hippel-Lindau (VHL) E3 ubiquitin ligase and the hypoxia inducible factor (HIE) alpha subunit with in vitro nanomolar affinities. J. Med. Chem. 2014, 57, 8657-8663.
(23) Buckley, D. L.; Gustafson, J. L.; van Molle, I.; Roth, A. G.; Tae, H. S.; Gareiss, P. C.; Jorgensen, W. L.; Ciulli, A.; Crews, C. M. Small-molecule inhibitors of the interaction between the E3 ligase VHL and HIE 1 alpha. Angew. Chem. Int. Ed. 2012, 57, 11463-11467.
(24) Buckley, D. L.; van Molle, I.; Gareiss, P. C.; Tae, H. S.; Michel, J.; Noblin, D. J.; Jorgensen, W. L.; Ciulli, A.; Crews, C. M. Targeting the von Hippel-Lindau E3 ubiquitin ligase using small molecules to disrupt the VHL/HIF-1 alpha interaction. J. Am. Chem. Soc. 2012, 134, 4465-4468.
(25) Luzzio, M. J.; Autry, C. L.; Bhattacharya, S. K.; Freeman-Cook, K. D.; Hayward, M. M.; Hulford, C. A.; Nelson, K. L.; Xiao, J.; Zhao, X. Sulfonyl amide derivatives for the treatment of abnormal cell growth; WO Patent, 2008 (WO2008/129380).
(26) Bondeson, D. P.; Smith, B. E.; Burslem, G. M.; Buhimschi, A. D.; Hines, J.; Jaime-Figueroa, S.; Wang, J.; Hamman, B. D.; Ishchenko, A.; Crews, C. M. Lessons in PROTAC Design from Selective Degradation with a Promiscuous Warhead. Cell Chem. Biol. 2017.
(27) Douglass, E. F.; Miller, C. J.; Sparer, G.; Shapiro, H.; Spiegel, D. A. A comprehensive mathematical model for three-body binding equilibria. J. Am. Chem. Soc. 2013, 135, 6092-6099.
(28) Lahlou, H.; Sanguin-Gendreau, V.; Frame, M. C.; Muller, W. J. Focal adhesion kinase contributes to proliferative potential of ErbB2 mammary tumour cells but is dispensable for ErbB2 mammary tumour induction in vivo. Breast Cancer Res 2012, 14, R₃₆.
(29) Castoria, G.; D'Amato, L.; Ciociola, A.; Giovannelli, P.; Giraldi, T.; Sepe, L.; Paolella, G.; Barone, M. V.; Migliaccio, A.; Auricchio, F. Androgen-Induced Cell Migration: Role of Androgen Receptor/Filamin A Association. PLoS ONE 2011, 6, e17218.
(30) Castoria, G.; Auricchio, F.; Migliaccio, A. Extranuclear partners of androgen receptor: at the crossroads of proliferation, migration, and neuritogenesis. FASEB J. 2017, 31, 1289-1300.
(31) Hutchinson, J. A.; Shanware, N. P.; Chang, H.; Tibbetts, R. S. Regulation of Ribosomal Protein S6 Phosphorylation by Casein Kinase 1 and Protein Phosphatase 1. J. Biol. Chem. 2011, 286, 8688-8696.
(32) Lepin, E. J.; Zhang, Q.; Zhang, X.; Jindra, P. T.; Hong, L. S.; Ayele, P.; Peralta, M. V. P.; Gjertson, D. W.; Kobashigawa, J. A.; Wallace, W. D. et al. Phosphorylated S6 Ribosomal Protein: A Novel Biomarker of Antibody-Mediated Rejection in Heart Allografts. Am J Transplant 2006, 6, 1560-1571.
(33) Valeur, E.; Jimonet, P. New Modalities, Technologies, and Partnerships in Probe and Lead Generation: Enabling a Mode-of-Action Centric Paradigm. J. Med. Chem. 2018, DOI: 10.1021/acs.jmedchem.8b00378.
(34) Waldmann, H.; Valeur, E.; Guéret, S. M.; Adihou, H.; Gopalakrishnan, R.; Lemurell, M.; Grossmann, T. N.; Plowright, A. T. New Modalities for Challenging Targets in Drug Discovery. Angew. Chem. Int. Ed. 2017, 56, 10294-10323.
(35) Cromm, P. M.; Spiegel, J.; Grossmann, T. N. Hydrocarbon Stapled Peptides as Modulators of Biological Function. ACS Chem. Biol. 2015, 10, 1362-1375.
(36) Spiegel, J.; Cromm, P. M.; Zimmermann, G.; Grossmann, T. N.; Waldmann, H. Small-molecule modulation of Ras signaling. Nat. Chem. Biol. 2014, 10, 613-622.
(37) Cromm, P. M.; Spiegel, J.; Grossmann, T. N.; Waldmann, H. Direct Modulation of Small GTPase Activity and Function. Angew. Chem. Int. Ed. 2015, 54, 13516-13537.
(38) Buckley, D. L.; Raina, K.; Darricarrere, N.; Hines, J.; Gustafson, J. L.; Smith, I. E.; Miah, A. H.; Harling, J. D.; Crews, C. M. HaloPROTACS: Use of Small Molecule PROTACs to Induce Degradation of HaloTag Fusion Proteins. ACS Chem. Biol. 2015, 10, 1831-1837.
(39) Turk, B. E.; Jiang, H.; Liu, J. O. Binding of thalidomide to alpha1-acid glycoprotein may be involved in its inhibition of tumor necrosis factor alpha production. Proc. Natl. Acad. Sci. U.S.A. 1996, 93, 7552-7556.

What is claimed is:

1. A bifunctional compound having the chemical structure:
ULM-L-PTM,
or a pharmaceutically acceptable salt, enantiomer, stereoisomer, solvate, polymorph or prodrug thereof,
wherein:
the ULM is a small molecule E3 ubiquitin ligase binding moiety that binds an E3 ubiquitin ligase;
the PTM is a small molecule comprising a focal adhesion kinase protein targeting moiety; and
the L is a bond or a chemical linking moiety connecting the ULM and the PTM.

2. The bifunctional compound according to claim 1, wherein the E3 ubiquitin ligase binding moiety that targets an E3 ubiquitin ligase selected from the group consisting of Von Hippel-Lindau (VLM), cereblon (CLM), mouse double-minute homolog2 (MLM), and IAP (ILM).

3. The compound according to claim 1, wherein PTM is represented by: 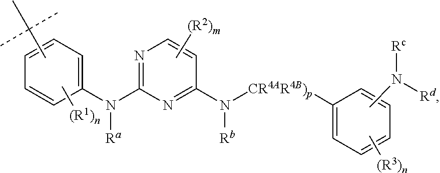
wherein:
each of R¹, R², R³, R^{4A}, R^{4B} is independently hydrogen, halogen, C₁-C₈ alkyl, C₁-C₈ haloalkyl, C₂-C₈ alkenyl, C₂-C₈ alkynyl, C₁-C₈ alkloxy, C₃-C₈ cycloalkyl, heterocycloalkyl, aryl, heteroaryl, primary amino, secondary amino, amido, carboxyl, acetyl or cyano;
each of R^{a}, R^{b}, R^{c}, and R^{d} is independently hydrogen, C₁-C₈ alkyl, C₁-C₈ haloalkyl, C₂-C₈ alkenyl, C₂-C₈ alkynyl, C₃-C₈ cycloalkyl, heterocycloalkyl, aryl, heteroaryl, or SO2-R^{e};
each R^{e} is independently C₁-C₄ alkyl;
each n is independently an integer from 0 to 4;
m is an integer from 0 to 2;
p is an integer from 1 to 8; and
the 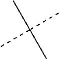
indicates the site of attachment of at least one of a linker, ULM, ULM′, CLM, CLM′, VLM, VLM′, ILM, ILM′, MLM, MLM′, or a combination thereof.

4. The compound according to claim 1, wherein the PTM is represented by: 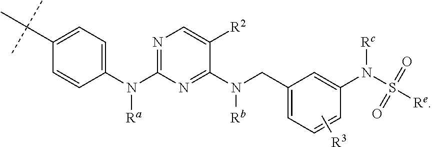
wherein:
R² is hydrogen, halogen, C₁-C₄ alkyl, or C₁-C₄ haloalkyl;
R³ is hydrogen, halogen, C₁-C₄ alkyl, C₁-C₄ alkoxy, or —CN;
each of R^{a}, R^{b}, and R^{c} are independently, hydrogen or C₁-C₄ alkyl;
R^{e} is C₁-C₁ alkyl; and
the 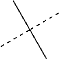
indicates the site of attachment of at least one of a linker, ULM, ULM′, CLM, CLM′, VLM, VLM′, ILM, ILM′, MLM, MLM′, or a combination thereof.

5. The compound according to claim 1, wherein PTM is represented by chemical structure: 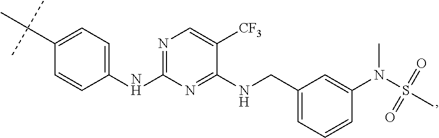
wherein the 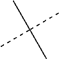
indicates the site of attachment of at least one of a linker, ULM, ULM′, CLM, CLM′, VLM, VLM′, ILM, ILM′, MLM, MLM′, or a combination thereof.

6. The compound according to claim 1, wherein ULM is a Von Hippel-Lindau (VHL) ligase-binding moiety (VLM) with a chemical structure represented by: 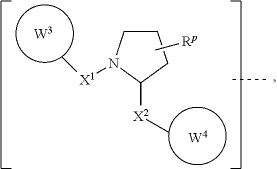
wherein:
X¹, X² are each independently selected from the group of a bond, O, NR^{Y3}, CR^{Y3}R^{Y4}, C═O, C═S, SO, and SO₂;
R^{Y3}, R^{Y4} are each independently selected from the group of H, optionally substituted linear or branched C₁₋₆ alkyl, optionally substituted C₁₋₆ alkoxyl optionally;
R^{p} is 0, 1, 2, or 3 groups independently selected from the group H, halo, —OH, C₁₋₃ alkyl, C═O;
W³ is selected from the group of an optionally substituted T, an optionally substituted -T-N(R^{1a}R^{1b})X³, an optionally substituted -T-N(R^{1a}R^{1b}), an optionally substituted -T-Aryl, an optionally substituted -T-Heteroaryl, an optionally substituted T-biheteroaryl, an optionally substituted -T-Heterocycle, an optionally substituted -T-biheterocycle, an optionally substituted —NR¹-T-Aryl, an optionally substituted —NR¹-T-Heteroaryl, or an optionally substituted —NR¹-T-Heterocycle;
X³ is C═O, R¹, R^{1a}, R^{1b};
each of R¹, R^{1a}, R^{1b} is independently selected from the group consisting of H, linear or branched C₁-C₆ alkyl group optionally substituted by 1 or more halo or —OH groups, R^{Y3}C═O, R^{Y3}C═S, R^{Y3}SO, R^{Y3}SO₂, N(R^{Y3}R^{Y4})C═O, N(R^{Y3}R^{Y4})C═S, N(R^{Y3}R^{Y4})SO, and N(R^{Y3}R^{Y4})SO₂;
T is selected from the group of an optionally substituted alkyl, —(CH₂)ₙ— group, wherein each one of the methylene groups is optionally substituted with one or two substituents selected from the group of halogen, methyl, optionally substituted alkoxy, a linear or branched C₁-C₆ alkyl group optionally substituted by 1 or more halogen, C(O) NR¹R^{1a}, or NR¹R^{1a} or R¹ and R^{1a} are joined to form an optionally substituted heterocycle, or —OH groups or an amino acid side chain optionally substituted; and
n is 0 to 6,
W⁴ is 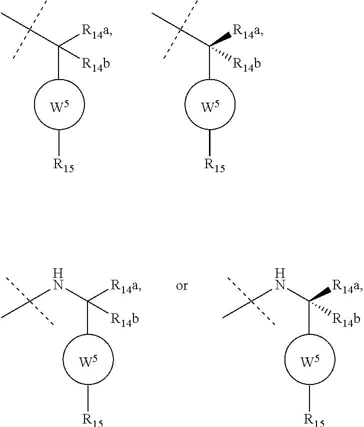
R₁₄ₐ, R_{14b}, are each independently selected from the group of H, haloalkyl, or optionally substituted alkyl;
W⁵ is selected from the group of an optionally substituted phenyl or an optionally substituted 5-10 membered heteroaryl,
R₁₅ is selected from the group of H, halogen, CN, OH, NO₂, NR₁₄ₐR_{14b}, OR₁₄ₐ, CONR₁₄ₐR_{14b}, NR₁₄ₐCOR_{14b}, SO₂NR₁₄ₐR_{14b}, NR₁₄ₐSO₂R_{14b}, optionally substituted alkyl, optionally substituted haloalkyl, optionally substituted haloalkoxy, optionally substituted aryl, optionally substituted heteroaryl, optionally substituted cycloalkyl, or optionally substituted cycloheteroalkyl; and
the dashed line indicates the site of attachment of at least one PTM, another ULM (ULM′) or a chemical linker moiety coupling at least one PTM or a ULM′ or both to ULM.

7. The compound according to claim 1, wherein ULM is a Von Hippel-Lindau (VHL) ligase-binding moiety (VLM) with a chemical structure represented by: 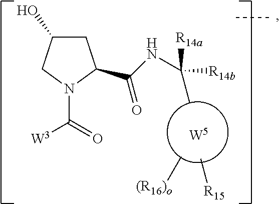
wherein:
W³ is selected from the group of an optionally substituted aryl, optionally substituted heteroaryl, or 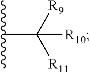
R₉ and R₁₀ are independently hydrogen, optionally substituted alkyl, optionally substituted cycloalkyl, optionally substituted hydroxyalkyl, optionally substituted heteroaryl, or haloalkyl, or R₉, R₁₀, and the carbon atom to which they are attached form an optionally substituted cycloalkyl;
R₁₁ is selected from the group of an optionally substituted heterocyclic, optionally substituted alkoxy, optionally substituted heteroaryl, optionally substituted aryl, 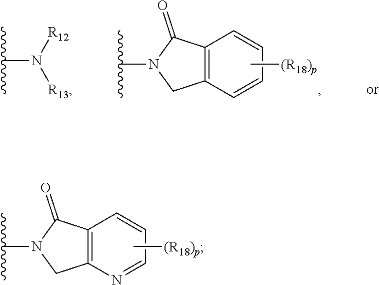
R₁₂ is selected from the group of H or optionally substituted alkyl;
R₁₃ is selected from the group of H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl;
R₁₄ₐ, R_{14b}, are each independently selected from the group of H, haloalkyl, or optionally substituted alkyl;
W⁵ is selected from the group of an optionally substituted phenyl or an optionally substituted 5-10 membered heteroaryl,
R₁₅ is selected from the group of H, halogen, CN, OH, NO₂, NR₁₄ₐR_{14b}, OR₁₄ₐ, CONR₁₄ₐR_{14b}, NR₁₄ₐCOR_{14b}, SO₂NR₁₄ₐR_{14b}, NR₁₄ₐSO₂R_{14b}, optionally substituted alkyl, optionally substituted haloalkyl, optionally substituted haloalkoxy, optionally substituted aryl, optionally substituted heteroaryl, optionally substituted cycloalkyl, or optionally substituted cycloheteroalkyl;
each R₁₆ is independently selected from the group of halo, optionally substituted alkyl, optionally substituted haloalkyl, hydroxy, or optionally substituted haloalkoxy;
o is 0, 1, 2, 3, or 4;
R₁₈ is independently selected from the group of halo, optionally substituted alkoxy, cyano, optionally substituted alkyl, haloalkyl, haloalkoxy or a linker; and
p is 0, 1, 2, 3, or 4, and wherein the dashed line indicates the site of attachment of at least one PTM, another ULM (ULM′) or a chemical linker moiety coupling at least one PTM or a ULM′ or both to ULM.

8. The compound according to claim 1, wherein the ULM has a chemical structure selected from the group of: 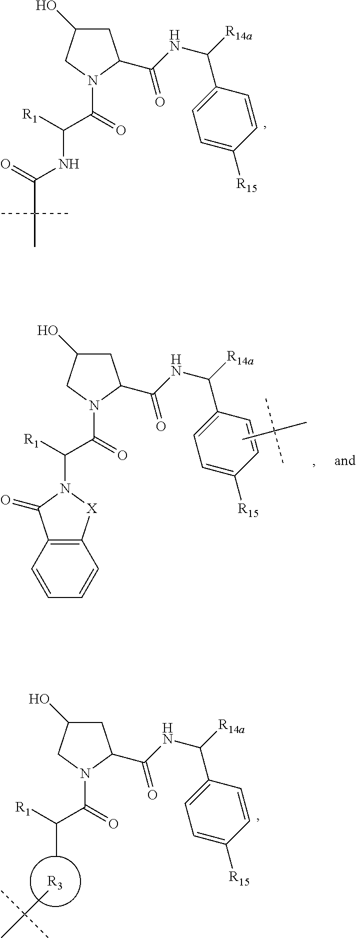
wherein:
R₁ is H, ethyl, isopropyl, tert-butyl, sec-butyl, cyclopropyl, cyclobutyl, cyclopentyl, or cyclohexyl; optionally substituted alkyl, optionally substituted hydroxyalkyl, optionally substituted heteroaryl, or haloalkyl;
R₁₄ₐ is H, haloalkyl, optionally substituted alkyl, methyl, fluoromethyl, hydroxymethyl, ethyl, isopropyl, or cyclopropyl;
R₁₅ is selected from the group consisting of H, halogen, CN, OH, NO₂, optionally substituted heteroaryl, optionally substituted aryl; optionally substituted alkyl, optionally substituted haloalkyl, optionally substituted haloalkoxy, optionally substituted cycloalkyl, or optionally substituted cycloheteroalkyl;
X is C, CH₂, or C═O
R₃ is absent or an optionally substituted 5 or 6 membered heteroaryl; and
the dashed line indicates the site of attachment of at least one PTM, another ULM (ULM′) or a chemical linker moiety coupling at least one PTM or a ULM′ or both to the ULM.

9. The compound according to claim 1, wherein the ULM is a cereblon E3 ligase-binding moiety (CLM) selected from the group consisting of a thalidomide, lenalidomide, pomalidomide, analogs thereof, isosteres thereof, or derivatives thereof.

10. The compound according to claim 9, wherein the CLM has a chemical structure represented by: 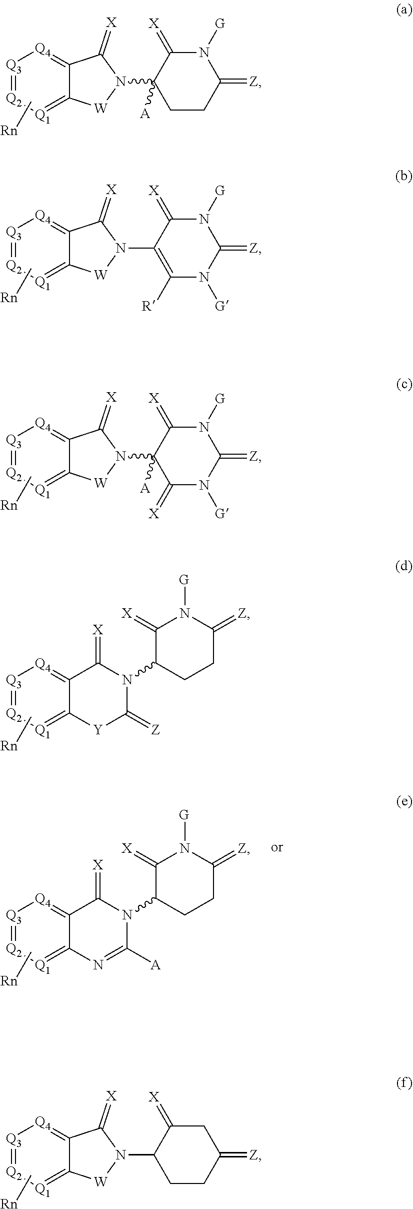
wherein:
W is selected from the group consisting of CH₂, CHR, C═O, SO₂, NH, and N-alkyl;
each X is independently selected from the group consisting of absent, O, S, and CH₂;
Y is selected from the group consisting of CH₂, —C═CR′, NH, N-alkyl, N-aryl, N-hetaryl, N-cycloalkyl, N-heterocyclyl, O, and S;
Z is selected from the group consisting of absent, O, S, and CH₂;
G and G′ are independently selected from the group consisting of H, optionally substituted linear or branched alkyl, OH, R′OCOOR, R′OCONRR″, CH₂-heterocyclyl optionally substituted with R′, and benzyl optionally substituted with R′;
Q₁, Q₂, Q₃, and Q₄ represent a carbon C substituted with a group independently selected from R′, N or N-oxide;
A is independently selected from the group H, optionally substituted linear or branched alkyl, cycloalkyl, Cl and F;
R comprises —CONR′R″, —OR′, —NR′R″, —SR′, —SO₂R′, —SO₂NR′R″, —CR′R″—, —CR′NR′R″—, (—CR′O)_{n′}R″, optionally substituted heterocyclyl, optionally substituted-aryl, optionally substituted-heteroaryl, -optionally substituted linear or branched alkyl, optionally substituted-cycloalkyl, -optionally substituted heterocyclyl, —P(O)(OR′)R″, —P(O)R′R″, —OP(O)(OR′)R″, —OP(O)R′R″, —C₁, —F, —Br, —I, —CF₃, —CN, —NR′SO₂NR′R″, —NR′CONR′R″, —CONR′COR″, —NR′C(═N—CN)NR′R″, —C(═N—CN)NR′R″, —NR′C(═N—CN)R″, —NR′C(═C—NO₂)NR′R″, —SO₂NR′COR″, —NO₂, —CO₂R′, —C(C═N—OR′)R″, —CR′═CR′R″, —CCR′, —S(C═O)(C═N—R′)R″, —SF₅ and —OCF₃ wherein at least one R is modified to be covalently joined to a PTM, a chemical linker group (L), a ULM, a CLM′ (e.g., CLM′ is an additional CLM that has the same or different structure as a first CLM), or a combination thereof;
n and n′ are independently an integer from 1 to 10;
R′ and R″ are independently selected from the group consisting of a bond, H, optionally substituted linear or branched alkyl, optionally substituted cycloalkyl, optionally substituted aryl, optionally substituted hetaryl, optionally substituted heterocyclyl; and [symbol]
represents a bond that may be stereospecific ((R) or (S)) or non-stereospecific.

11. The compound according to claim 9, wherein the CLM has a chemical structure represented by: 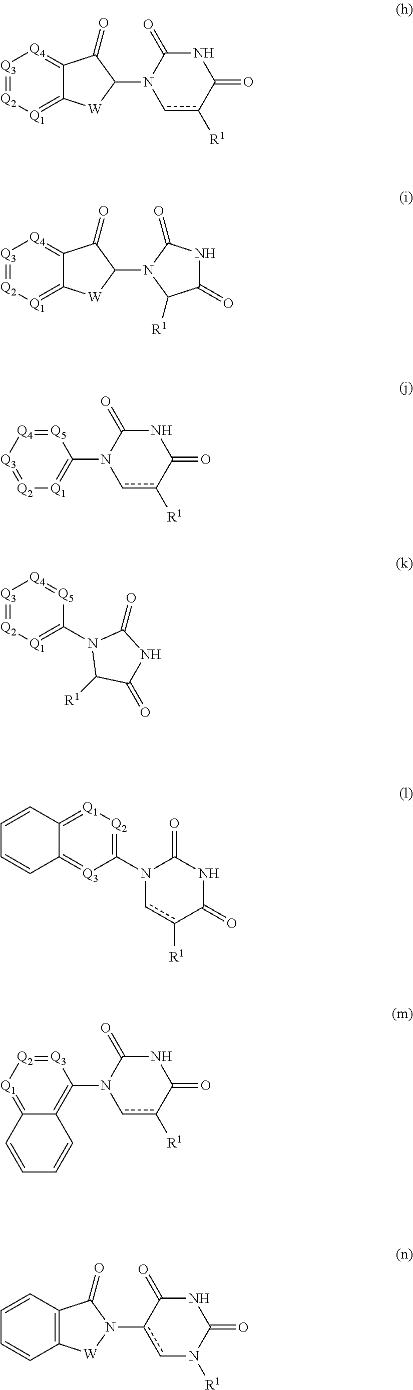 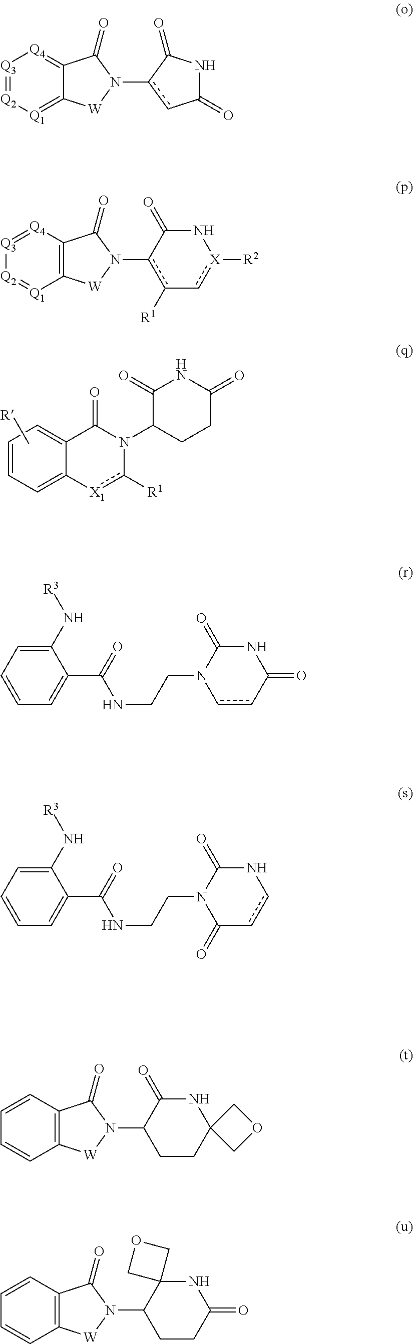 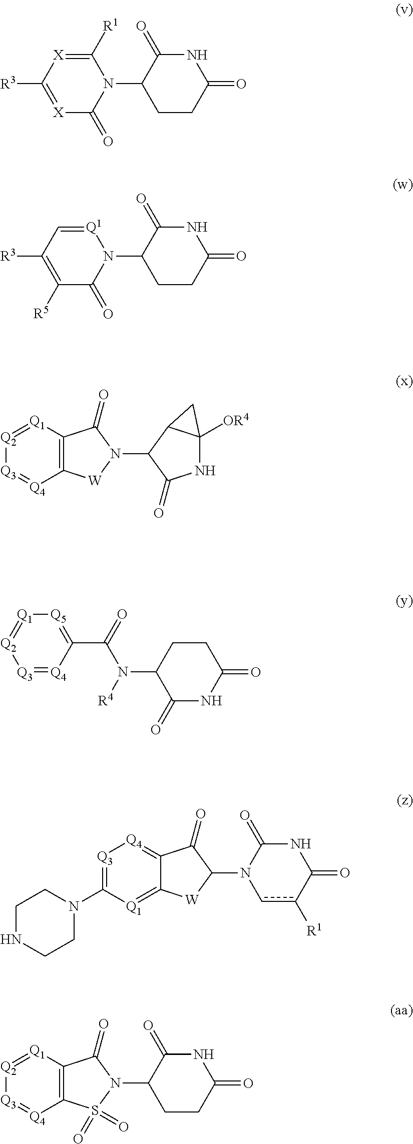 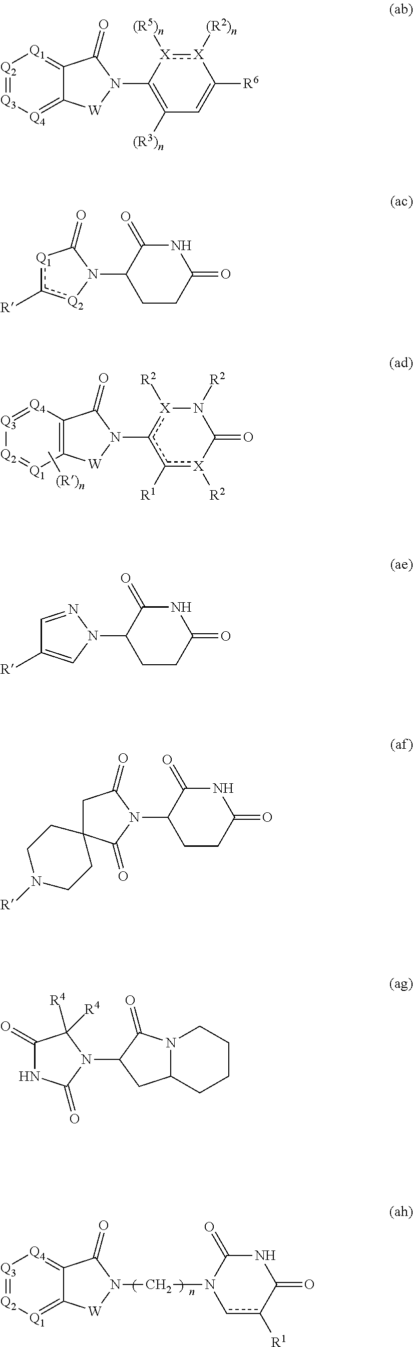 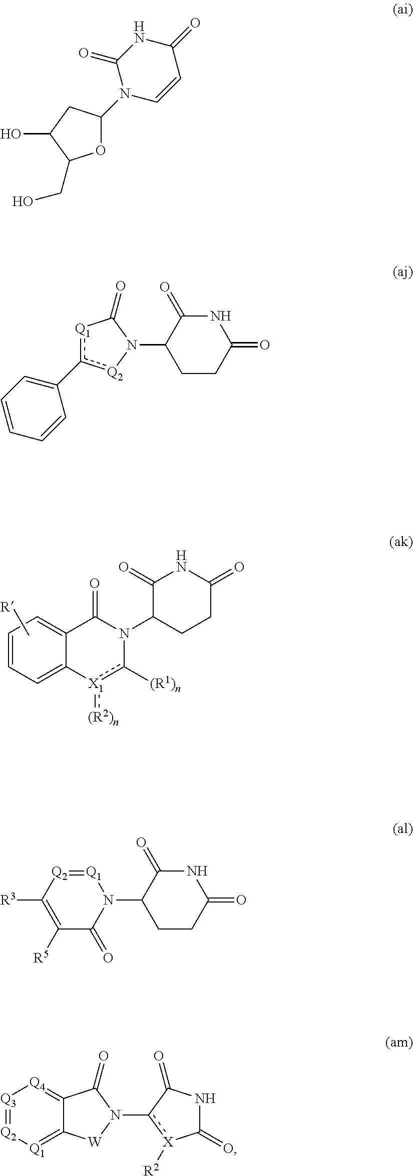
wherein:
W is independently selected from CH₂, CHR, C═O, SO₂, NH, and N-alkyl;
Q₁, Q₂, Q₃, Q₄, Q₅ are each independently represent a carbon C or N substituted with a group independently selected from R′, N or N-oxide;
R¹ is selected from absent, H, OH, CN, C1-C3 alkyl, C═O;
R² is selected from the group absent, H, OH, CN, C1-C3 alkyl, CHF₂, CF₃, CHO, C(═O)NH₂;
R³ is selected from H, alkyl (e.g., C1-C6 or C1-C3 alkyl), substituted alkyl (e.g., substituted C1-C6 or C1-C3 alkyl), alkoxy (e.g., C1-C6 or C1-C3 alkoxyl), substituted alkoxy (e.g., substituted C1-C6 or C1-C3 alkoxyl);
R⁴ is selected from H, alkyl, substituted alkyl;
R⁵ and R⁶ are each independently H, halogen, C(═O)R′, CN, OH, CF₃;
X is C, CH, C═O, or N;
X₁ is C═O, N, CH, or CH₂;
R′ is selected from H, halogen, amine, alkyl (e.g., C1-C3 alkyl), substituted alkyl (e.g., substituted C1-C3 alkyl), alkoxy (e.g., C1-C3 alkoxyl), substituted alkoxy (e.g., substituted C1-C3 alkoxyl), NR²R³, C(═O)OR², optionally substituted phenyl;
each n is independently an integer from 0 to 4; [symbol]
is a single or double bond; and
the CLM is covalently joined to a PTM, a chemical linker group (L), a ULM, CLM (or CLM′) or combination thereof.

12. The compound according to claim 1, wherein the ULM is a (MDM2) binding moiety (MLM) with a chemical moiety selected from the group consisting of a substituted imidazolines, a substituted spiro-indolinones, a substituted pyrrolidines, a substituted piperidinones, a substituted morpholinones, a substituted pyrrolopyrimidines, a substituted imidazolopyridines, a substituted thiazoloimidazoline, a substituted pyrrolopyrrolidinones, and a substituted isoquinolinones.

13. The compound according to claim 1, wherein the ULM is a IAP E3 ubiquitin ligase binding moiety (ILM) comprising the amino acids alanine (A), valine (V), proline (P), and isoleucine (I) or their unnatural mimetics.

14. The compound according to claim 1, wherein the ULM is a IAP E3 ubiquitin ligase binding moiety (ILM) comprising a AVPI tetrapeptide fragment or derivative thereof.

15. The compound according to claim 1, wherein the linker (L) comprises a chemical structural unit represented by the formula:
-(A^{L})_{q}-,
wherein:
-(A^{L})_{q}- is a group which is connected to the ULM, the PTM, or both;
q is an integer greater than or equal to 1;
each A is independently selected from the group consisting of, a bond, CR^{L1}R^{L2}, O, S, SO, SO₂, NR^{L3}, SO₂NR^{L3}, SONR^{L3}, CONR^{L3}, NR^{L3}CONR^{L4}, NR^{L3}SO₂NR^{L4}, CO, CR^{L1}═CR^{L2}, C≡C, SiR^{L1}R^{L2}, P(O)R^{L1}, P(O)OR^{L1}, NR^{L3}C(═NCN)NR^{L4}, NR^{L3}C(═NCN), NR^{L3}C(═CNO₂)NR^{L4}, C₃₋₁₁cycloalkyl optionally substituted with 0-6 R^{L1} and/or R^{L2} groups, C₃₋₁₁heterocyclyl optionally substituted with 0-6 R^{L1} and/or R^{L2} groups, aryl optionally substituted with 0-6 R^{L1} and/or R^{L2} groups, heteroaryl optionally substituted with 0-6 R^{L1} and/or R^{L2} groups, where R^{L1} or R^{L2}, each independently are optionally linked to other groups to form cycloalkyl and/or heterocyclyl moiety, optionally substituted with 0-4 R^{L5} groups; and
R^{L1}, R^{L2}, R^{L3}, R^{L4} and R^{L5} are, each independently, H, halo, C₁₋₈alkyl, OC₁₋₈alkyl, SC₁₋₈alkyl, NHC₁₋₈alkyl, N(C₁₋₈alkyl)₂, C₃₋₁₁cycloalkyl, aryl, heteroaryl, C₃₋₁₁heterocyclyl, OC₃₋₈cycloalkyl, SC₃₋₈cycloalkyl, NHC₃₋₈cycloalkyl, N(C₃₋₈cycloalkyl)₂, N(C₃₋₈cycloalkyl)(C₁₋₈alkyl), OH, NH₂, SH, SO₂C₁₋₈alkyl, P(O)(OC₁₋₈alkyl)(C₁₋₈alkyl), P(O)(OC₁₋₈alkyl)₂, CC—C₁₋₈alkyl, CCH, CH═CH(C₁₋₈alkyl), C(C₁₋₈alkyl)═CH(C₁₋₈alkyl), C(C₁₋₈alkyl)═C(C₁₋₈alkyl)₂, Si(OH)₃, Si(C₁₋₈alkyl)₃, Si(OH)(C₁₋₈alkyl)₂, COC₁₋₈alkyl, CO₂H, halogen, CN, CF₃, CHF₂, CH₂F, NO₂, SF₅, SO₂NHC₁₋₈alkyl, SO₂N(C₁₋₈alkyl)₂, SONHC₁₋₈alkyl, SON(C₁₋₈alkyl)₂, CONHC₁₋₈alkyl, CON(C₁₋₈alkyl)₂, N(C₁₋₈alkyl)CONH(C₁₋₈alkyl), N(C₁₋₈alkyl)CON(C₁₋₈alkyl)₂, NHCONH(C₁₋₈alkyl), NHCON(C₁₋₈alkyl)₂, NHCONH₂, N(C₁₋₈alkyl)SO₂NH(C₁₋₈alkyl), N(C₁₋₈alkyl) SO₂N(C₁₋₈alkyl)₂, NHSO₂NH(C₁₋₈alkyl), NHSO₂N(C₁₋₈alkyl)₂, NHSO₂NH₂.

16. The compound according to claim 15, wherein the unit A^{L} of linker (L) comprises a group represented by a general structure selected from the group consisting of:
—N(R)—(CH₂)ₘ—O(CH₂)ₙ—O(CH₂)ₒ—O(CH₂)ₚ—O(CH₂)_{q}—O(CH₂)ᵣ—OCH₂—,
—O—(CH₂)ₘ—O(CH₂)ₙ—O(CH₂)ₒ—O(CH₂)ₚ—O(CH₂)_{q}—O(CH₂)ᵣ—OCH₂—,
—O—(CH₂)ₘ—O(CH₂)ₙ—O(CH₂)ₒ—O(CH₂)ₚ—O(CH₂)_{q}—O(CH₂)ᵣ—O—;
—N(R)—(CH₂)ₘ—O(CH₂)ₙ—O(CH₂)ₒ—O(CH₂)ₚ—O(CH₂)_{q}—O(CH₂)ᵣ—O—;
—(CH₂)ₘ—O(CH₂)ₙ—O(CH₂)ₒ—O(CH₂)ₚ—O(CH₂)_{q}—O(CH₂)ᵣ—O—;
—(CH₂)ₘ—O(CH₂)ₙ—O(CH₂)ₒ—O(CH₂)ₚ—O(CH₂)_{q}—O(CH₂)ᵣ—OCH₂—; 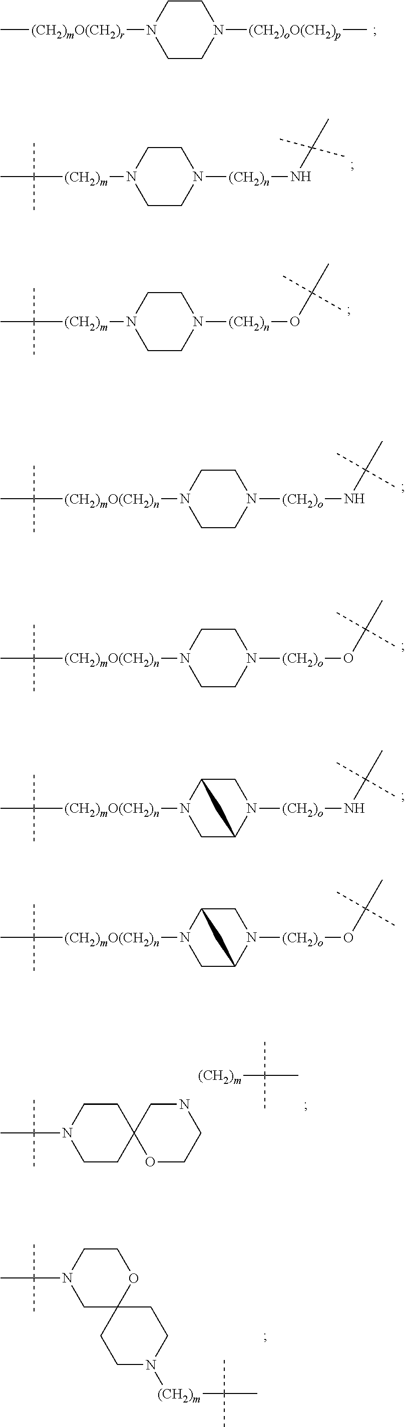 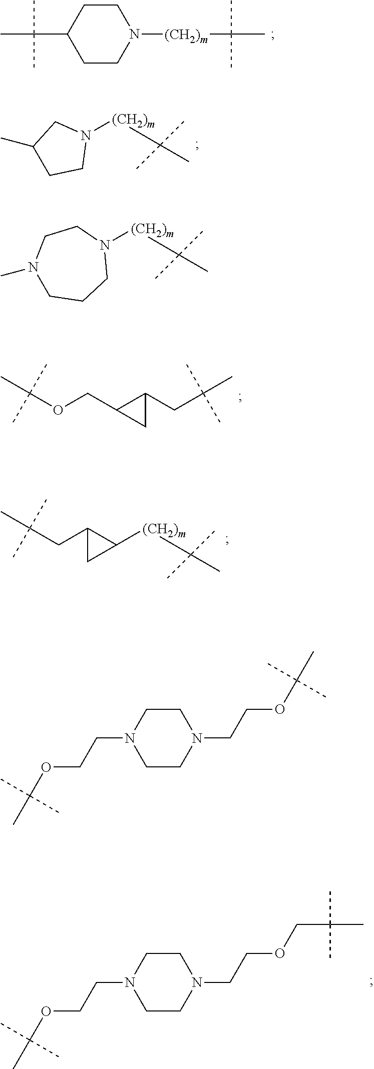 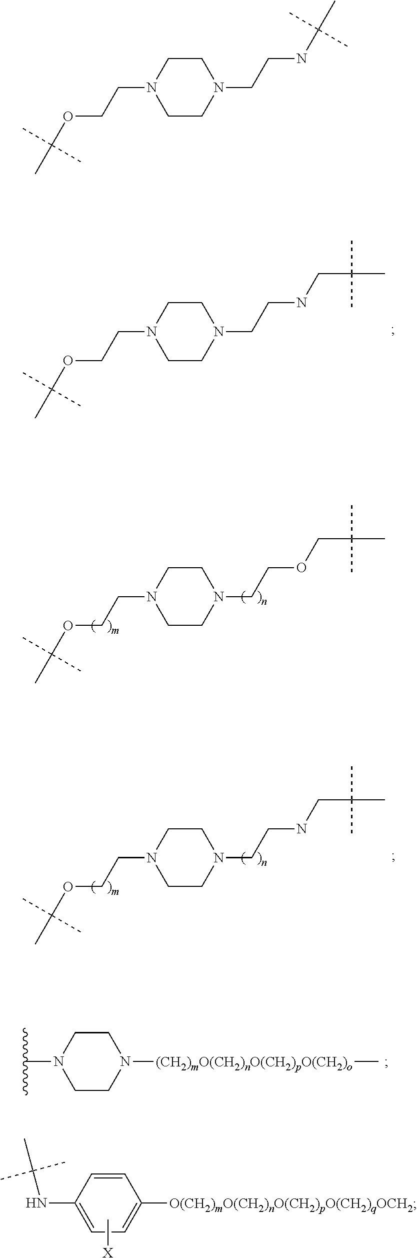 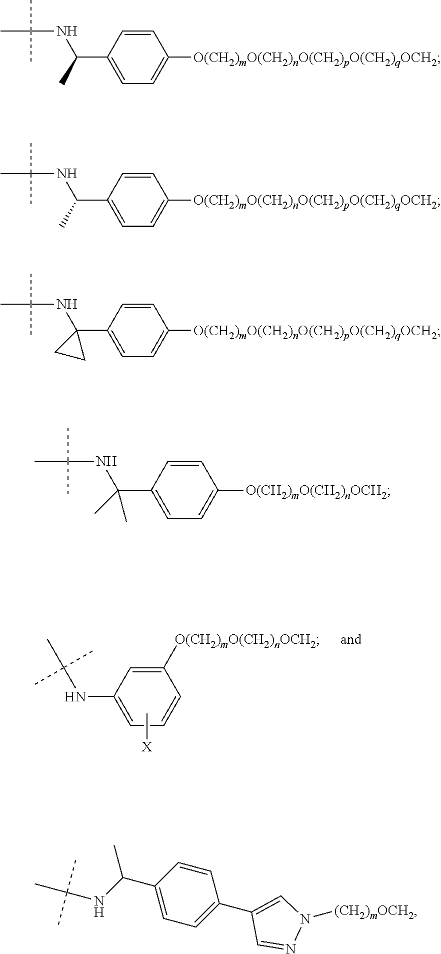
wherein each m, n, o, p, q, and r, is independently 0, 1, 2, 3, 4, 5, 6, 7, 8, 9, 10, 11, 12, 13, 14, 15, 16, 17, 18, 19, or 20 with the proviso that when the number is zero, there is no N—O or O—O bond, R is selected from the group H, methyl and ethyl, and X is selected from the group H and F;

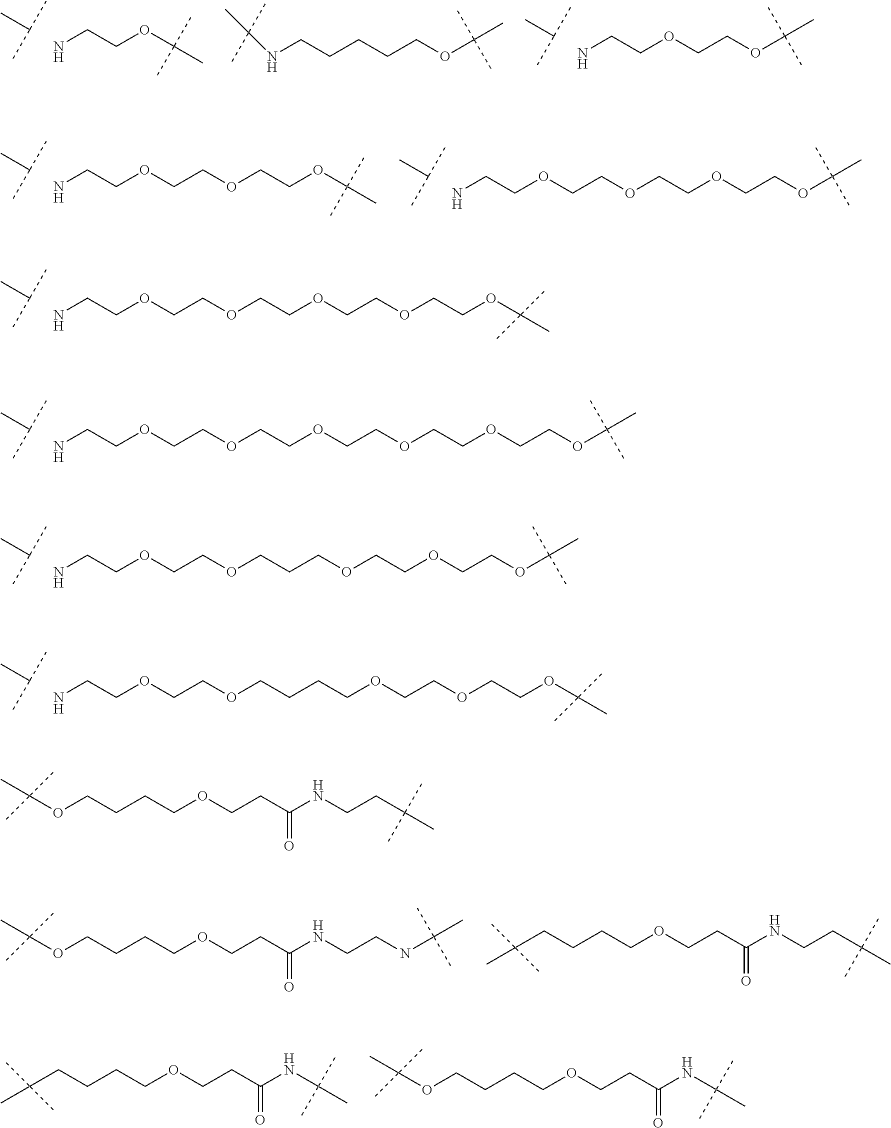

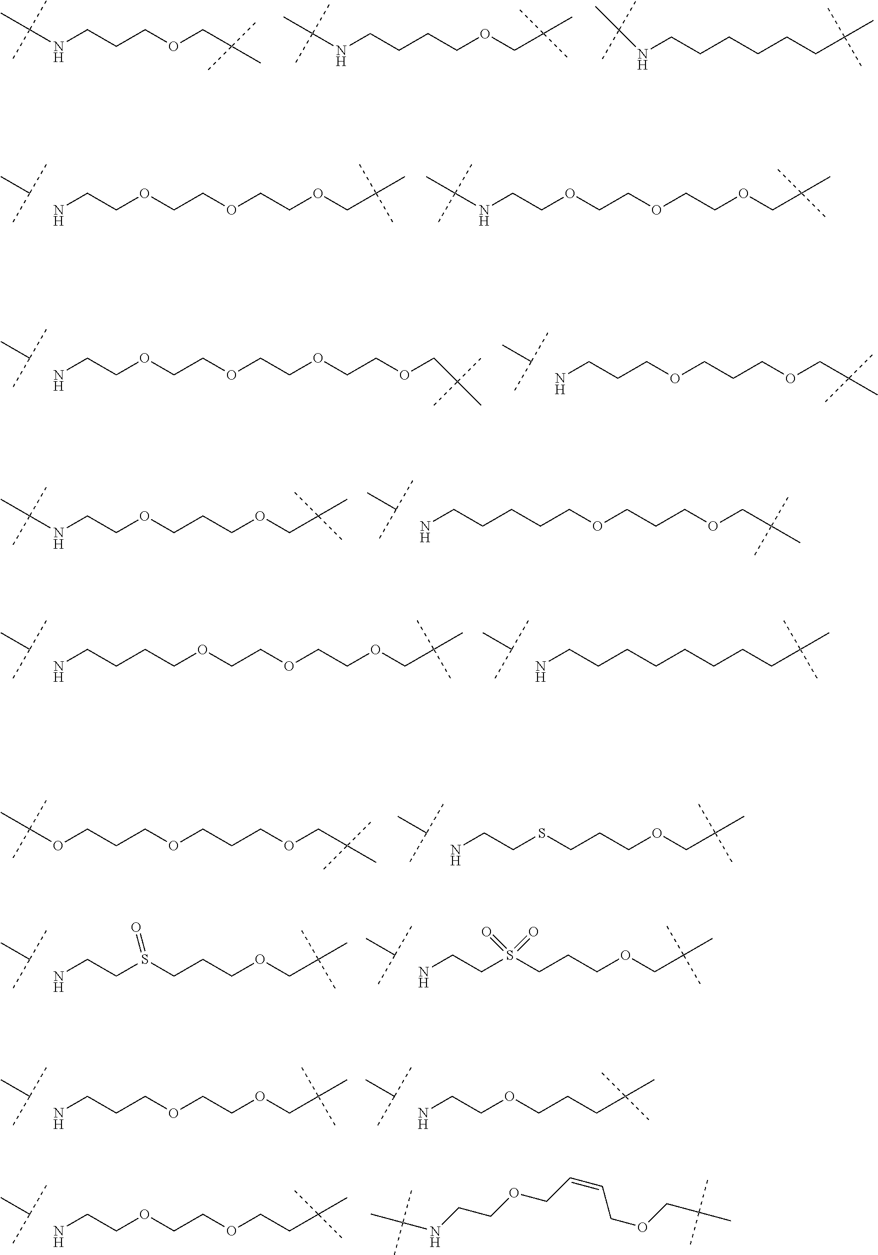

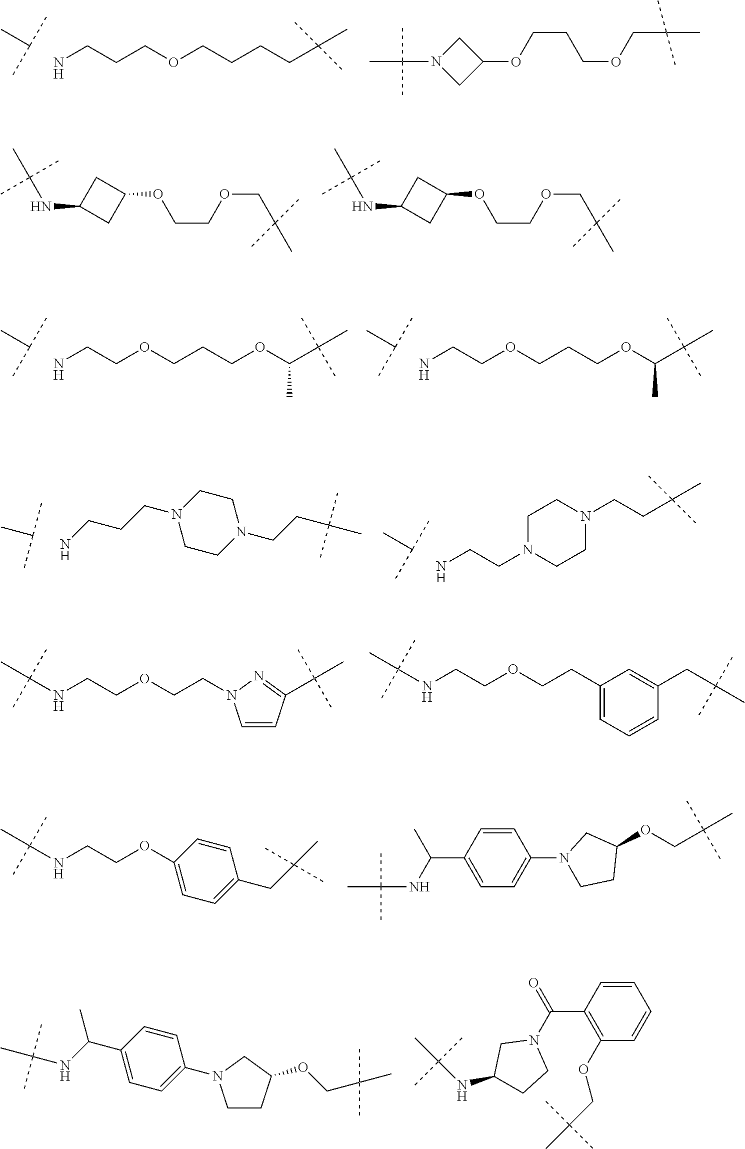

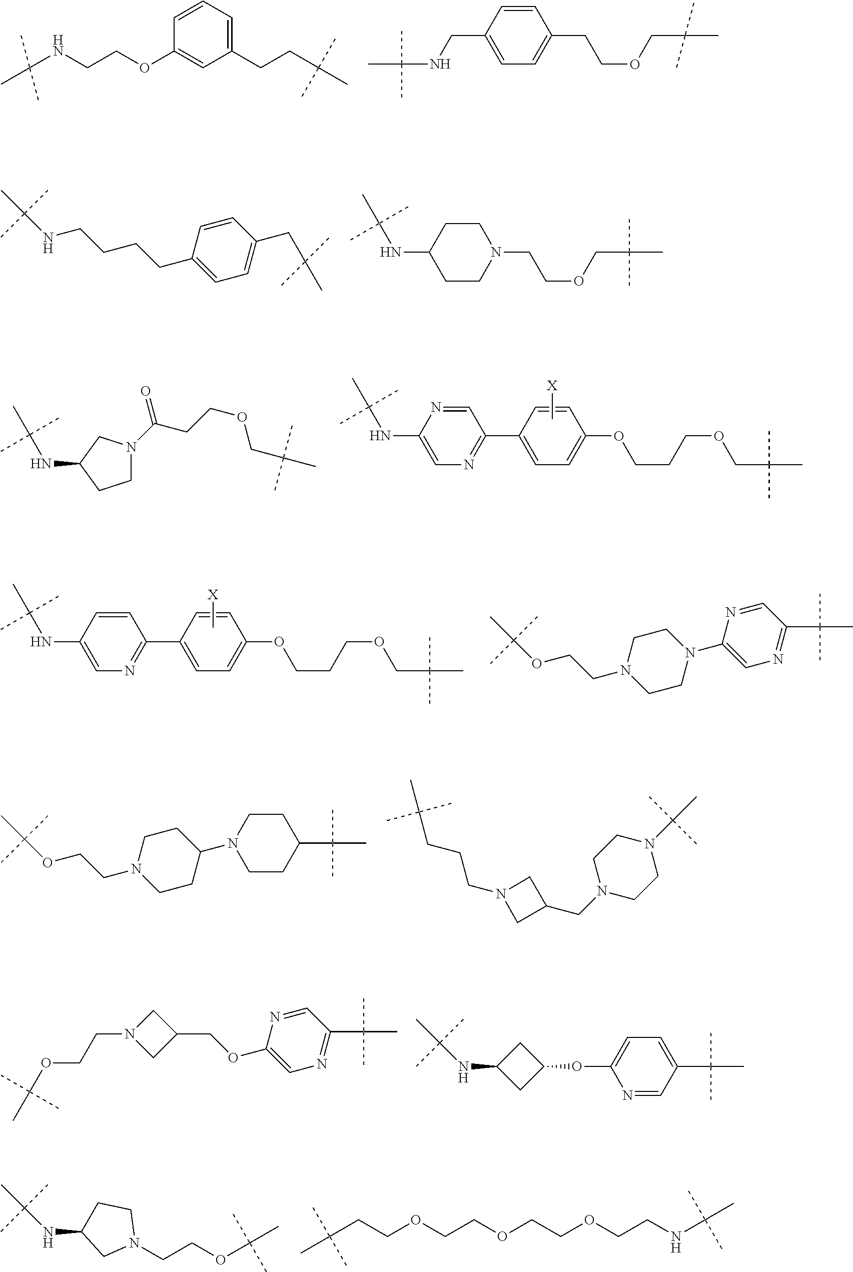

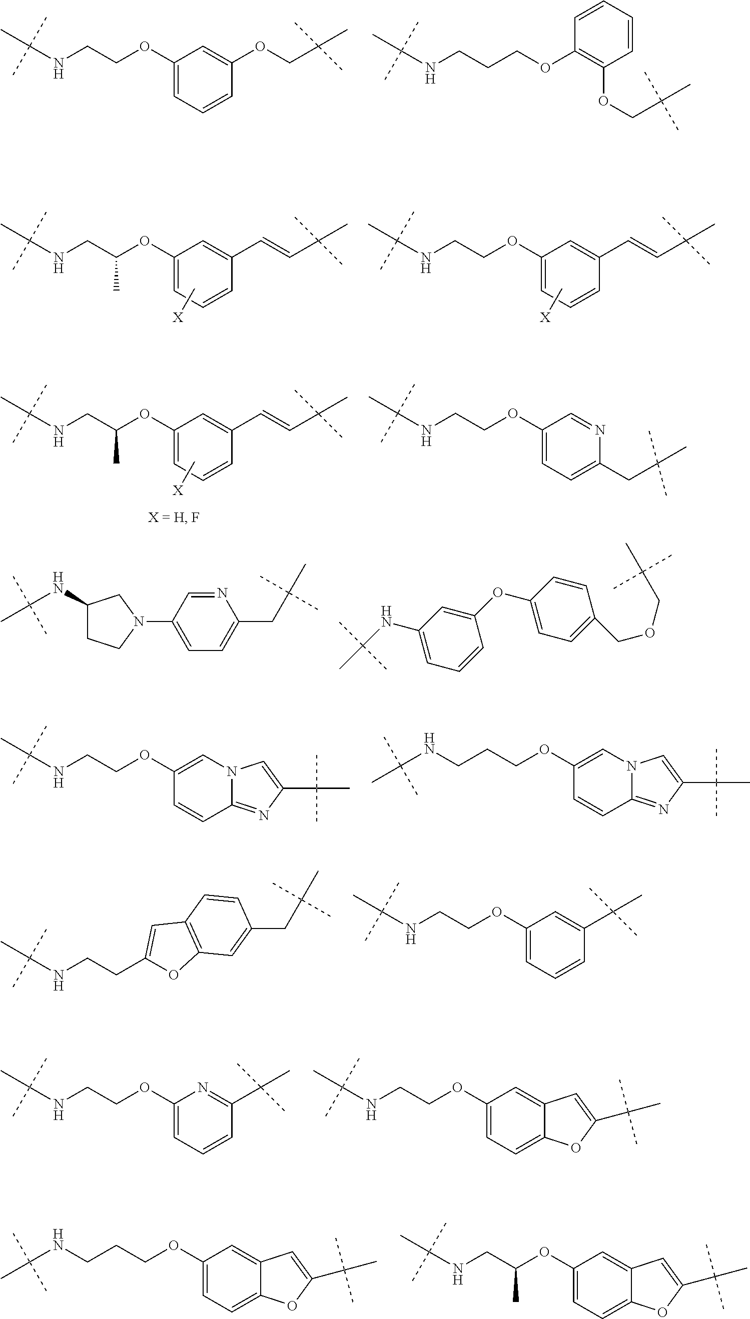

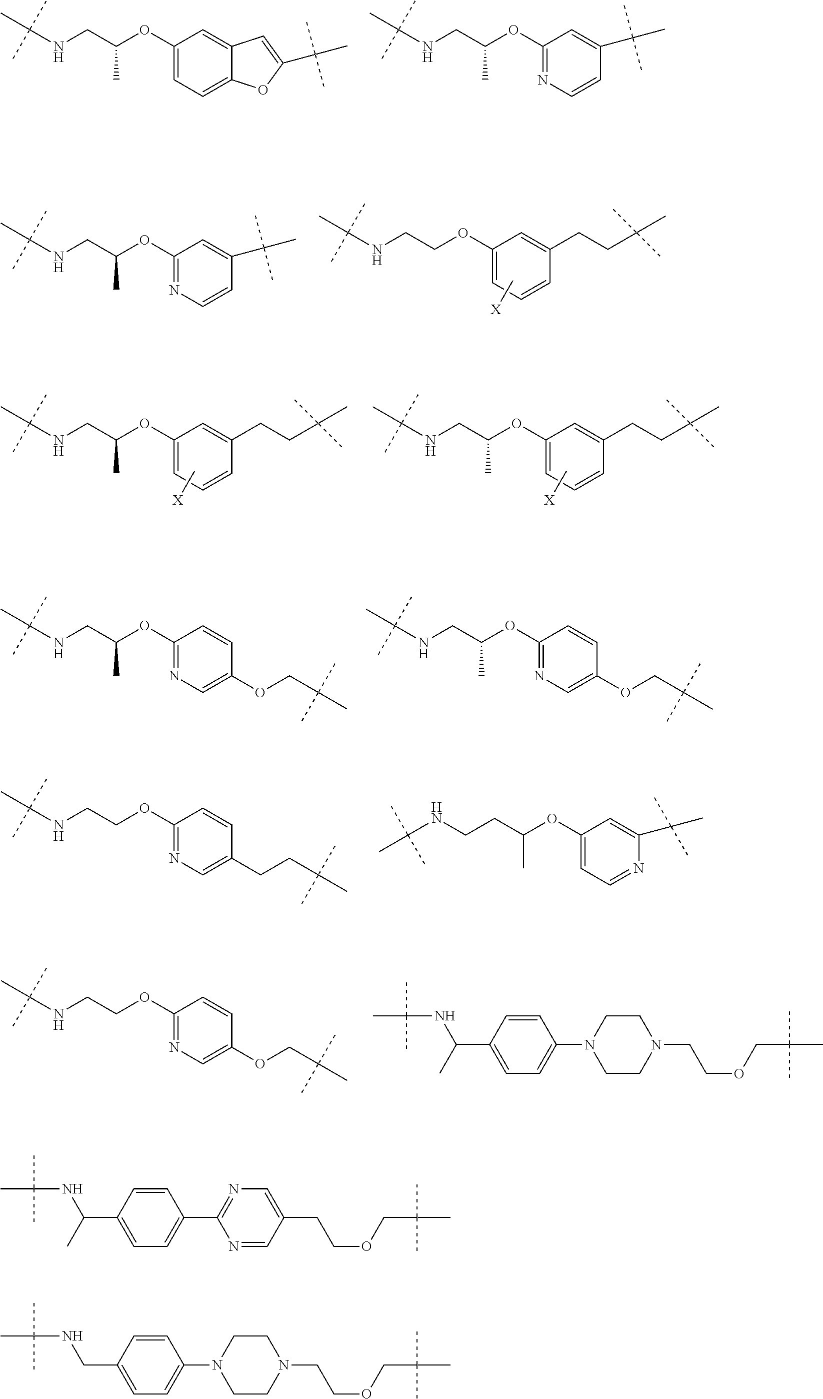

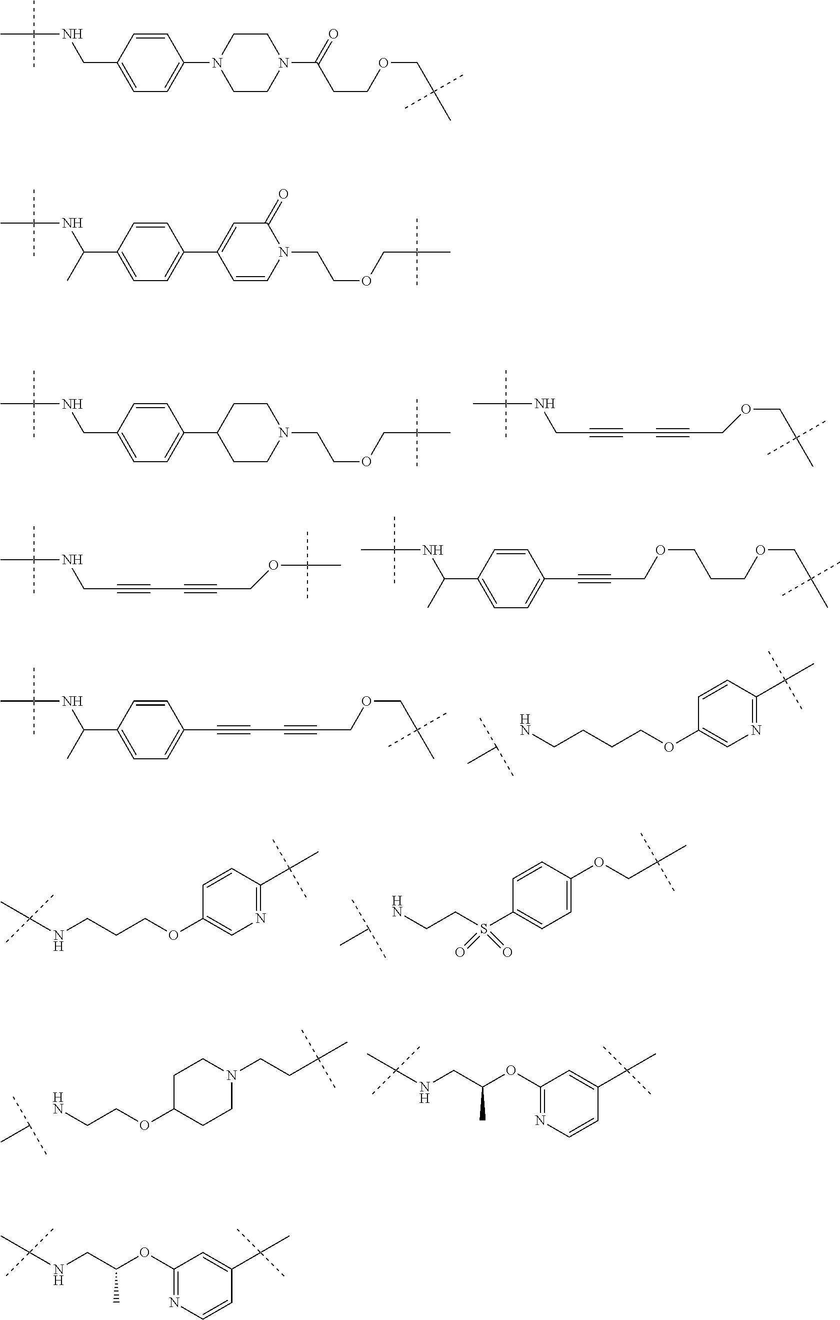

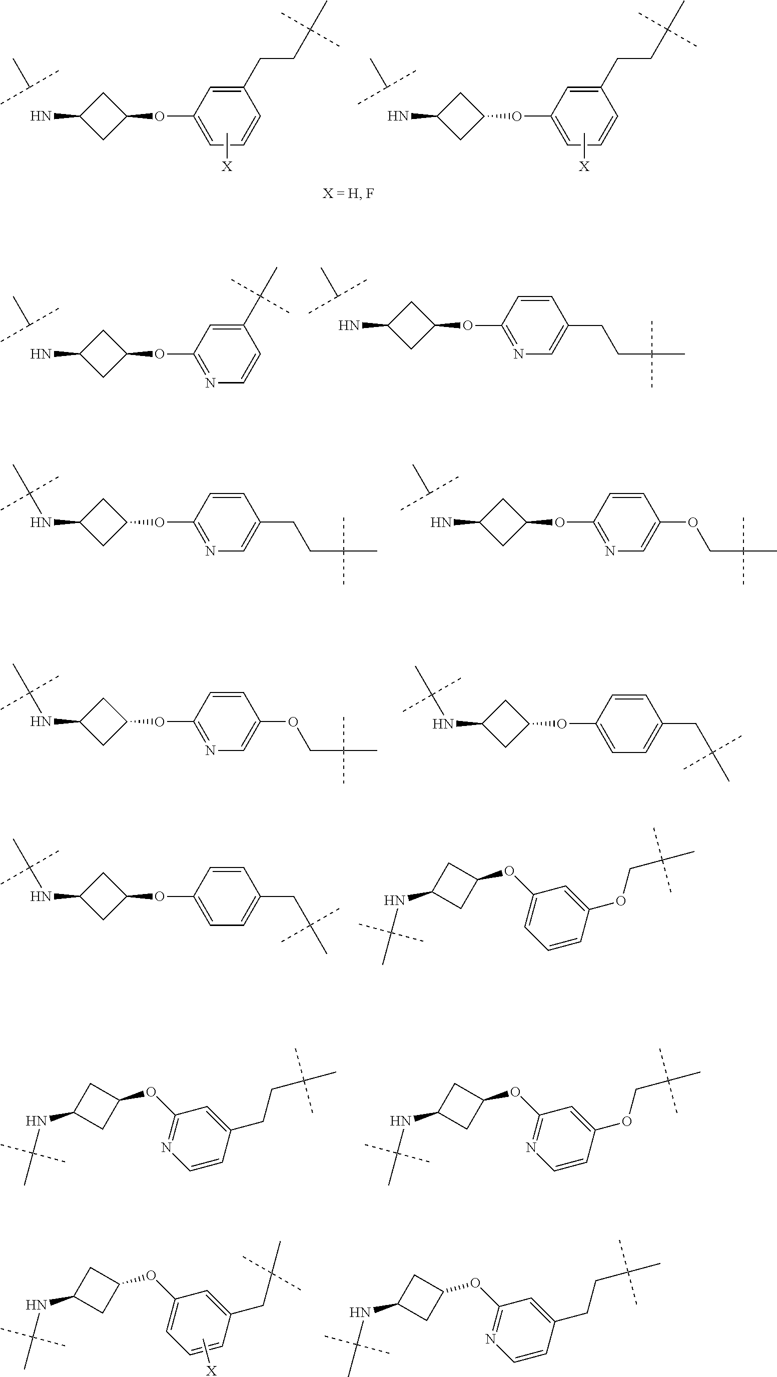

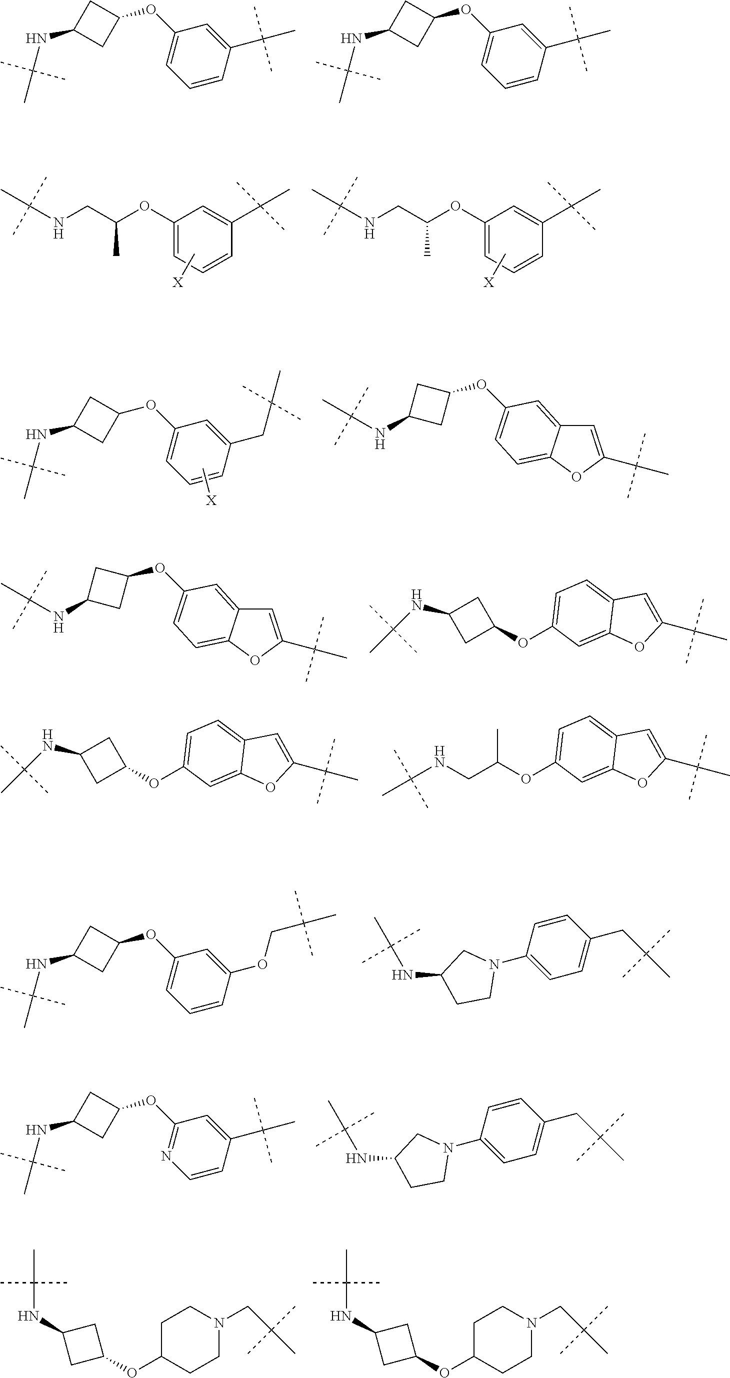

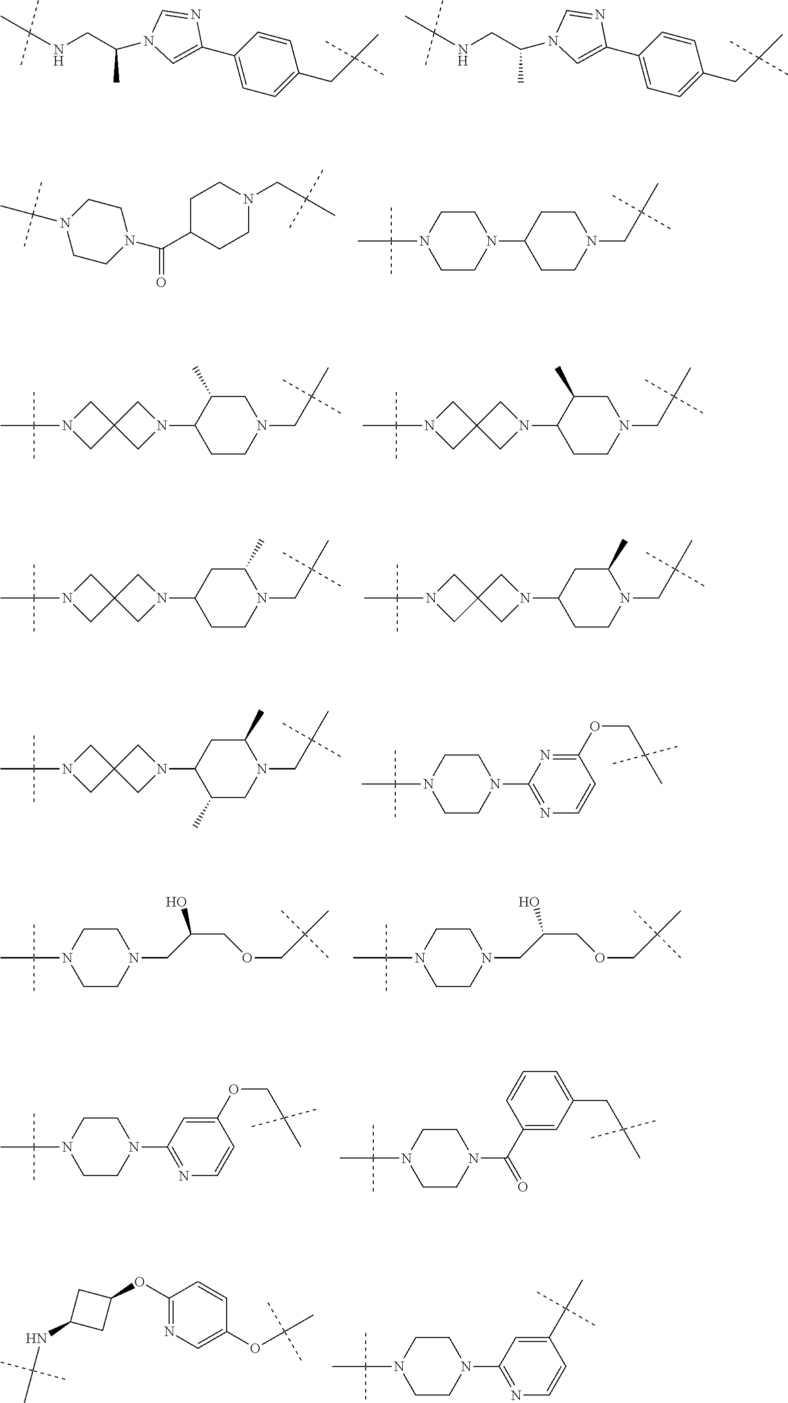

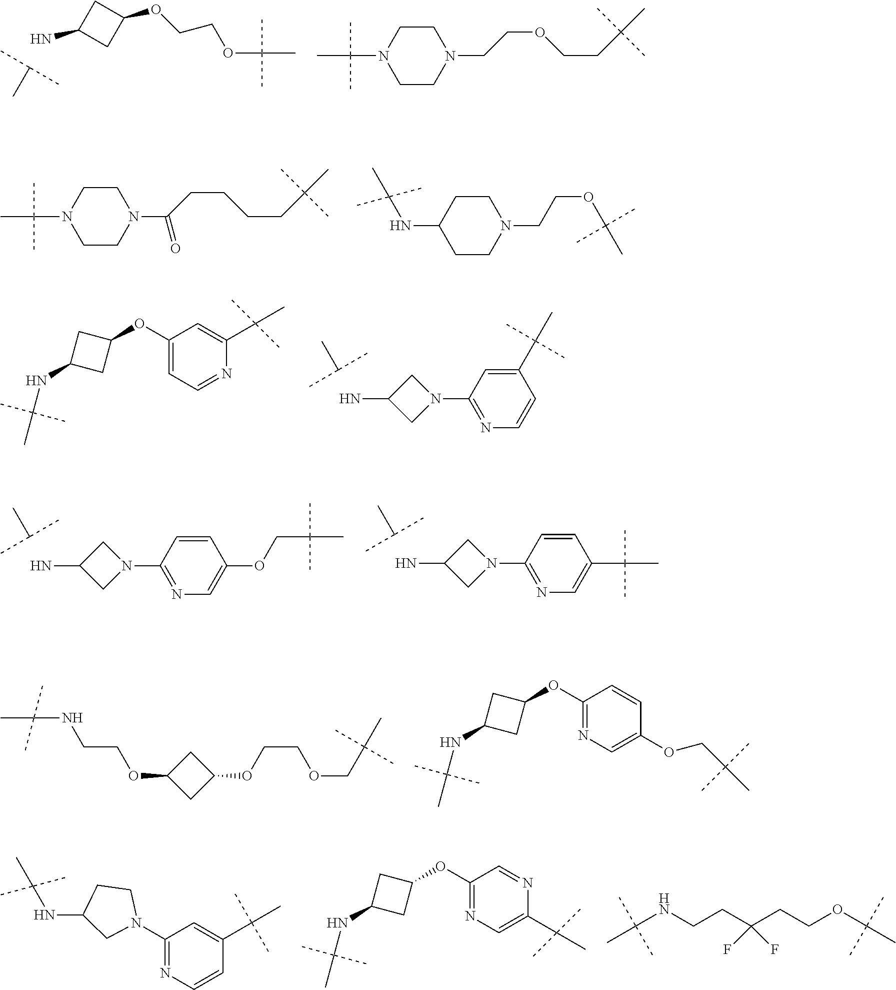

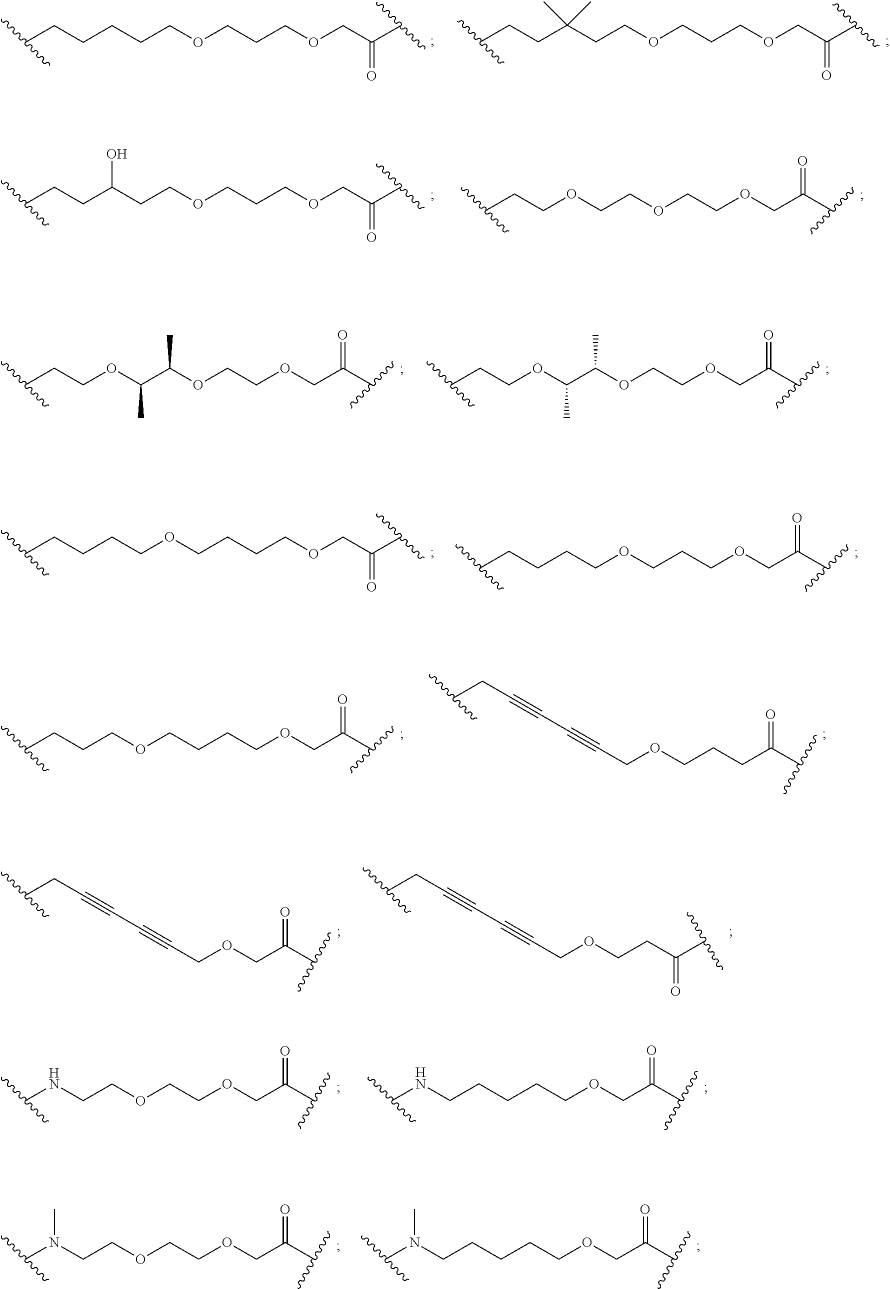

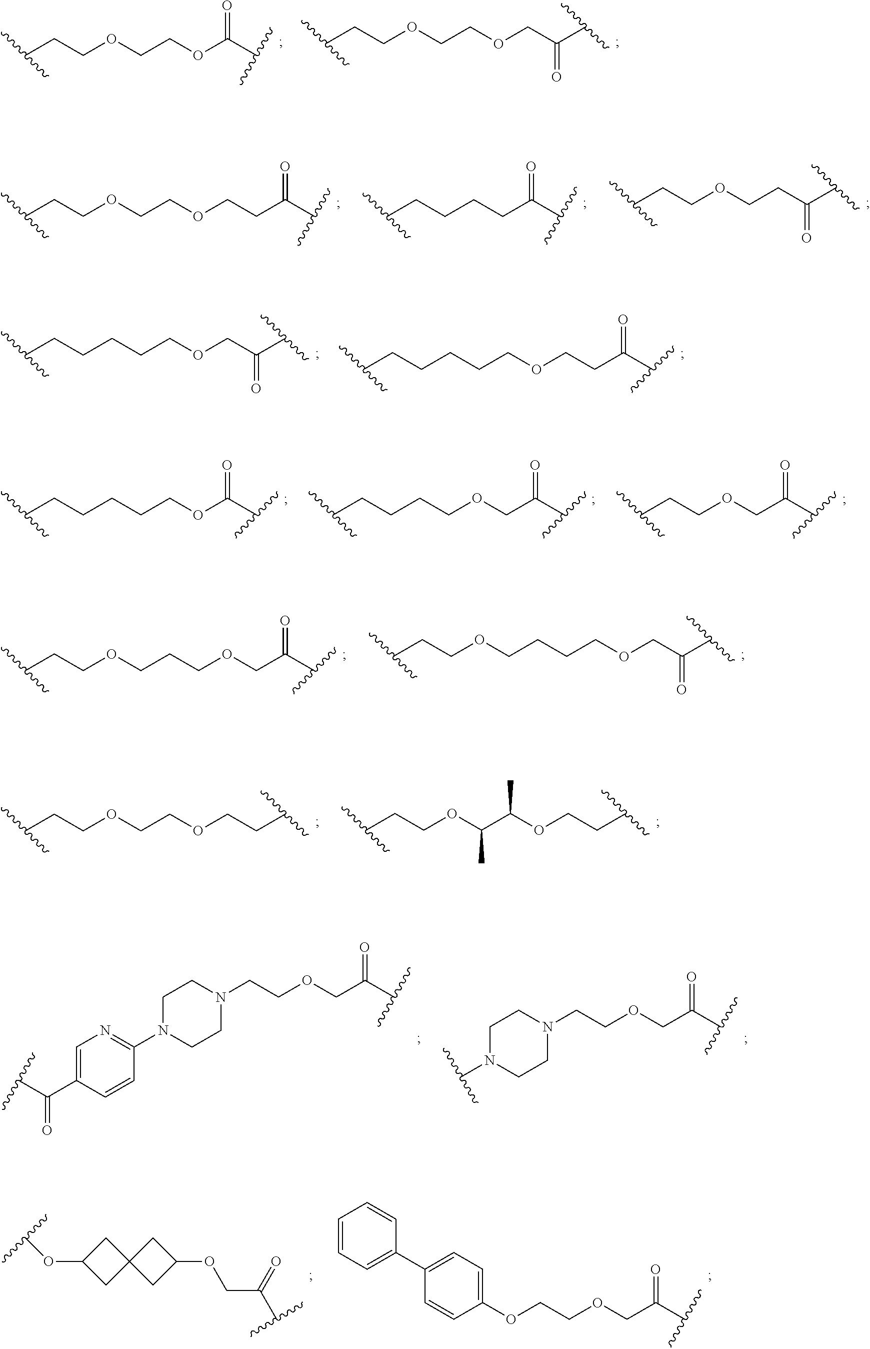

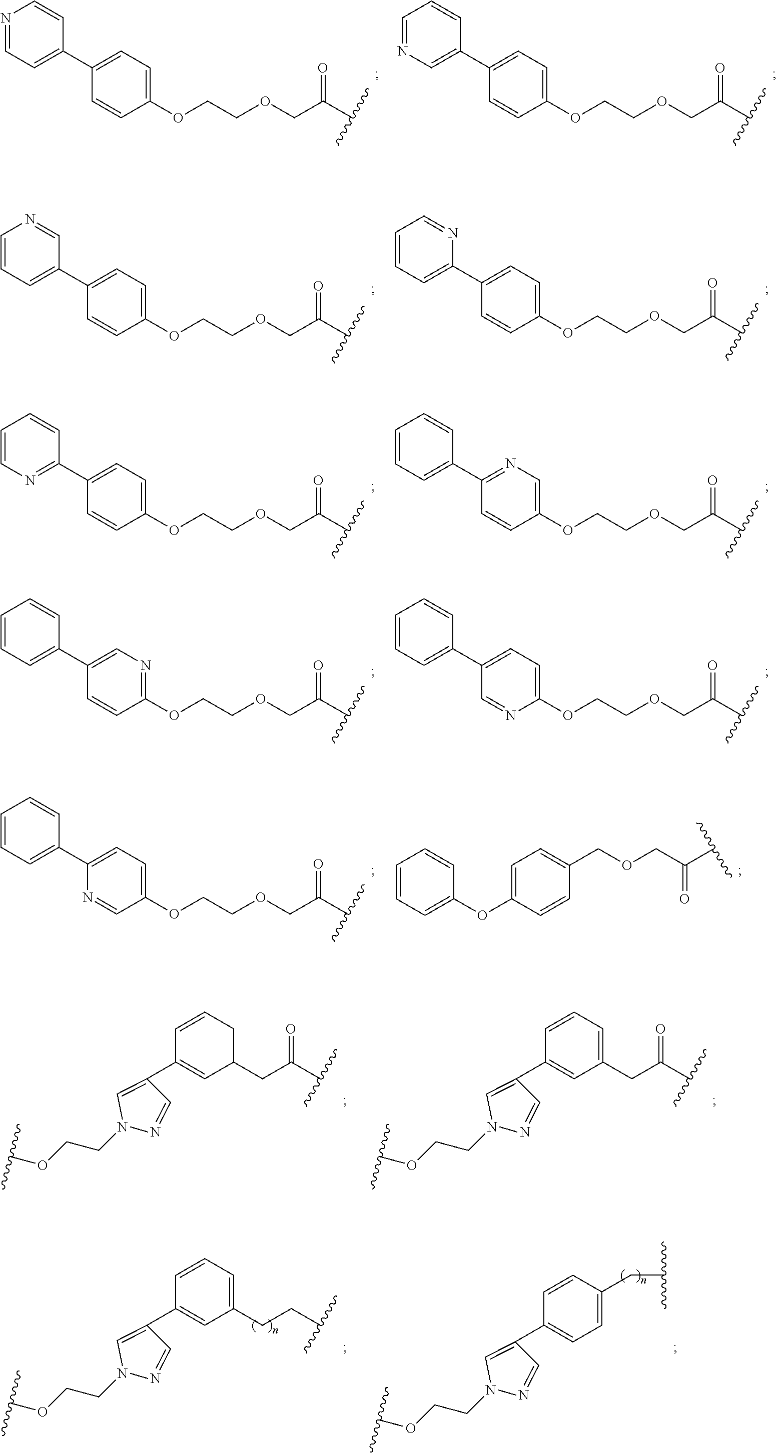

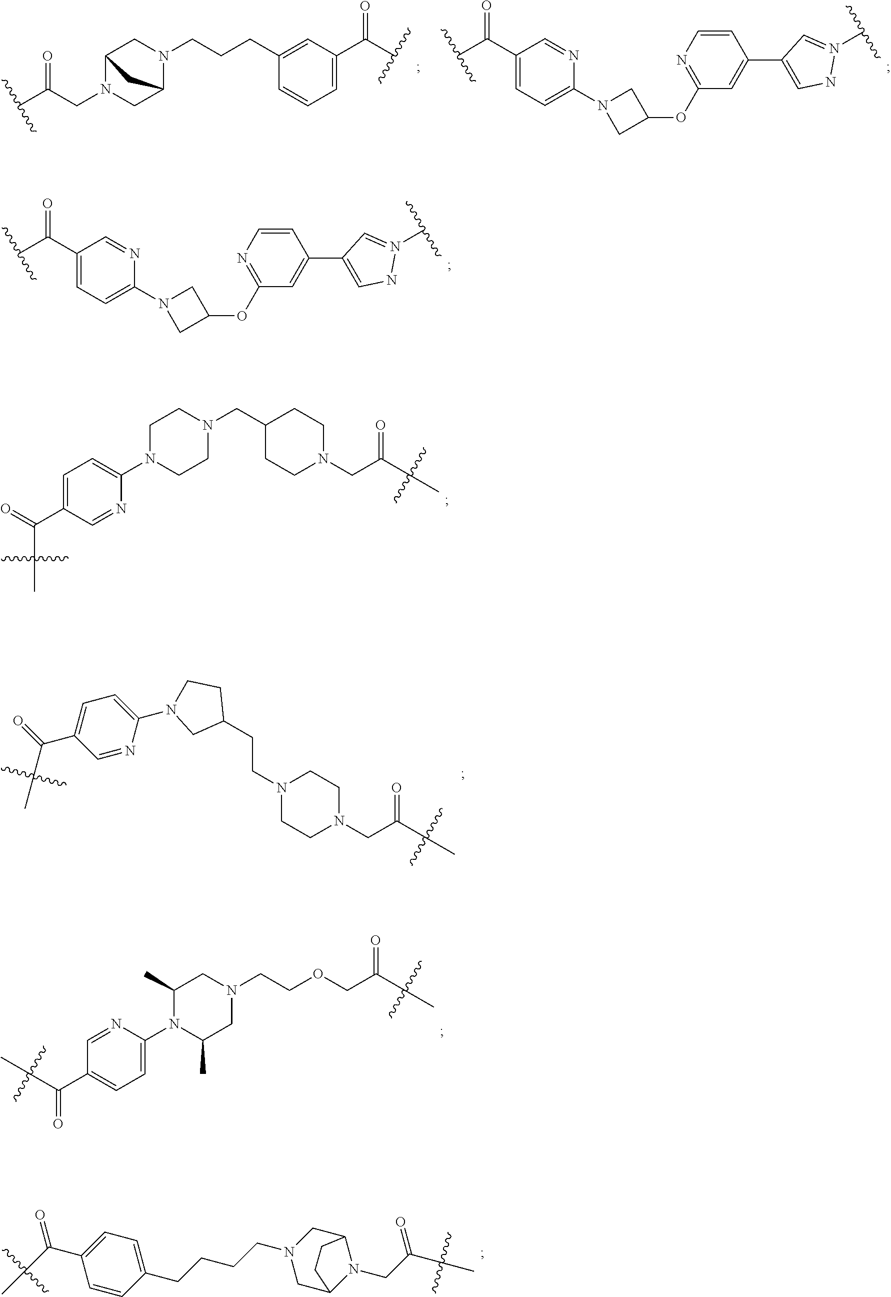

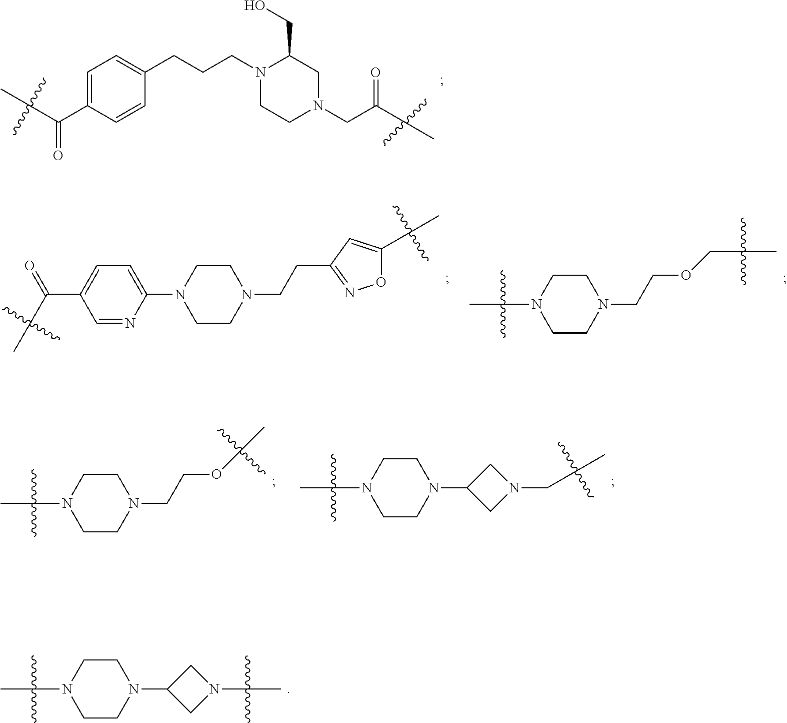 [SMILES: *C(=O)CN1CCN(CCCc2ccc(C(*)=O)cc2)[C@@H](CO)C1 ; *C(=O)c1ccc(N2CCN(CCc3cc(*)on3)CC2)nc1 ; *COCCN1CCN(*)CC1 ; *OCCN1CCN(*)CC1 ; *CN1CC(N2CCN(*)CC2)C1 ; *N1CCN(C2CN(*)C2)CC1]

17. The compound according to claim 15, wherein the unit A^{L} of linker (L) is selected from the group consisting of: 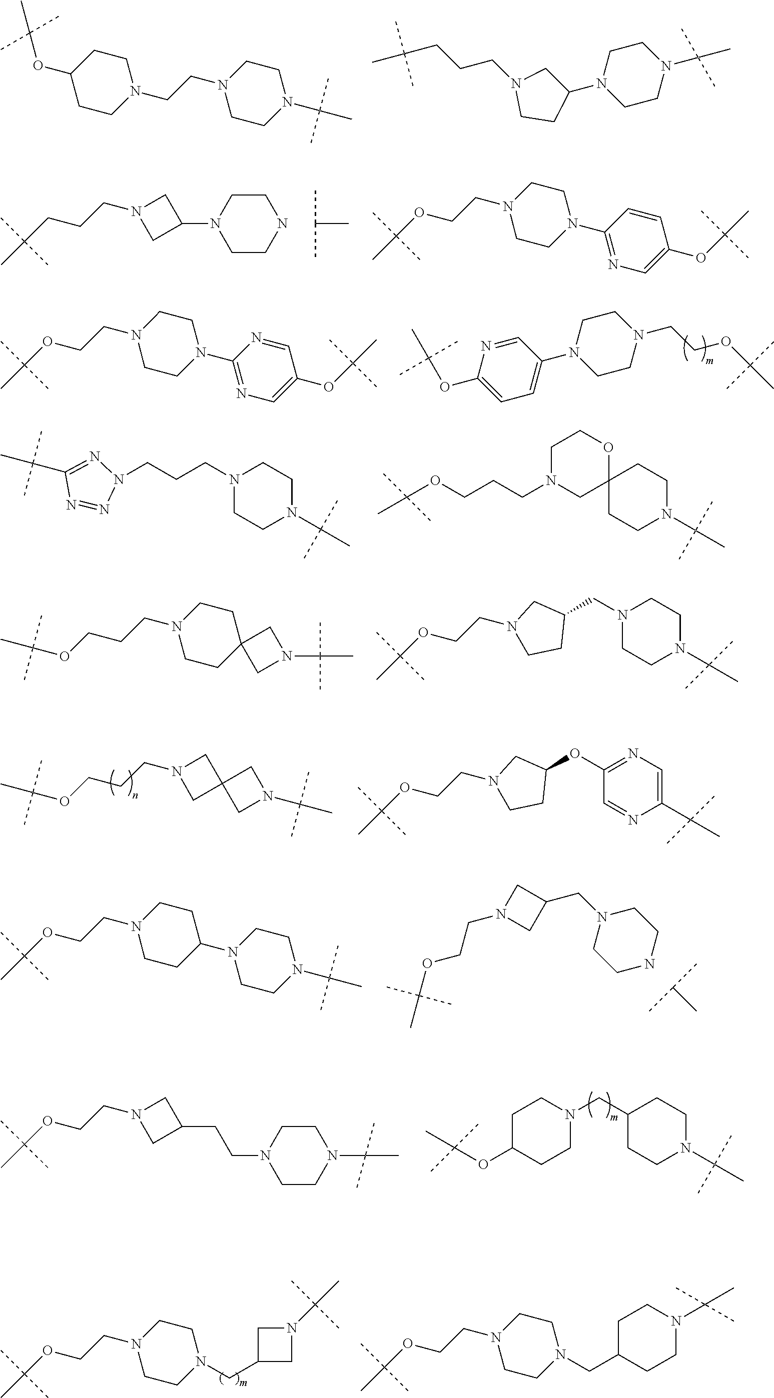 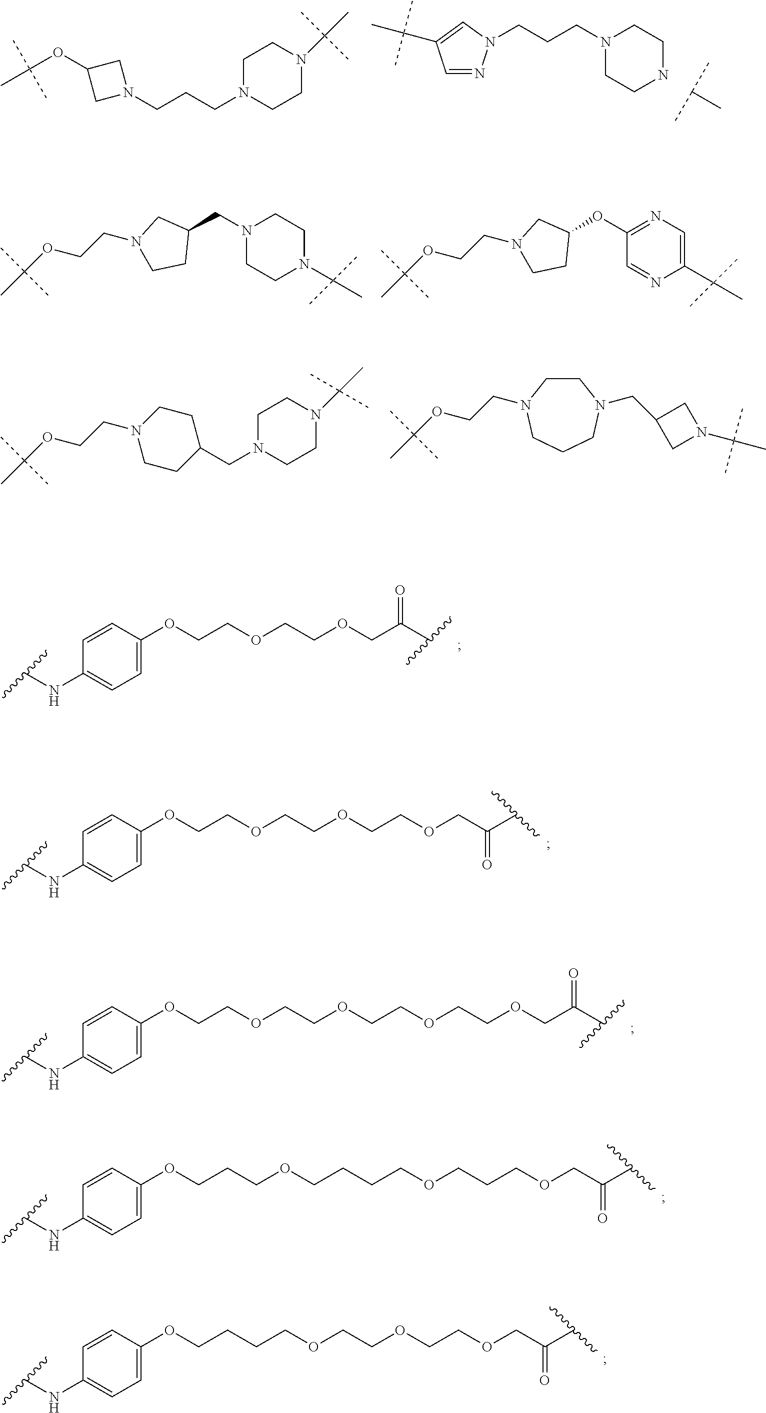 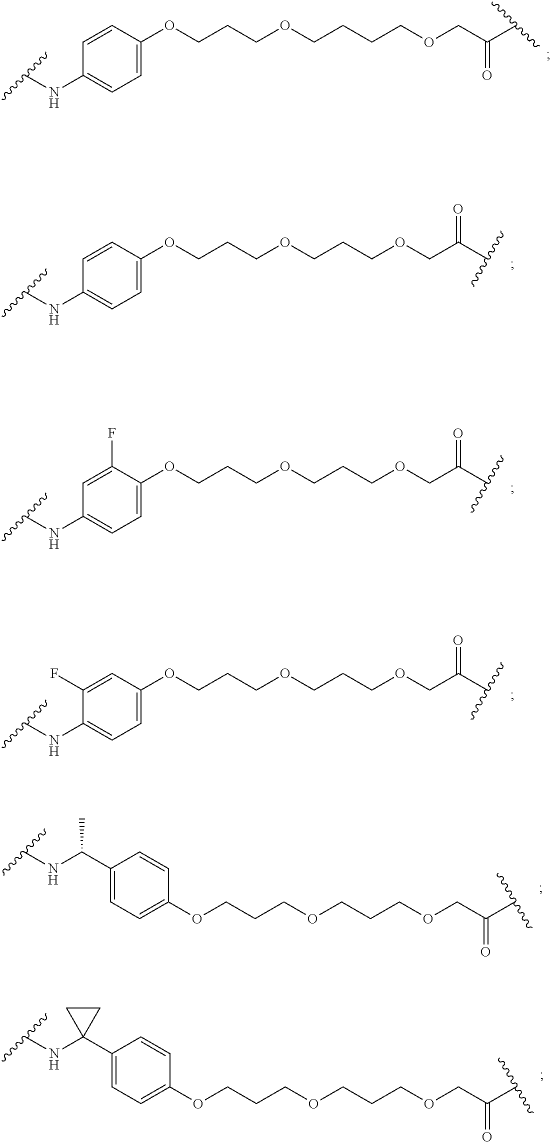 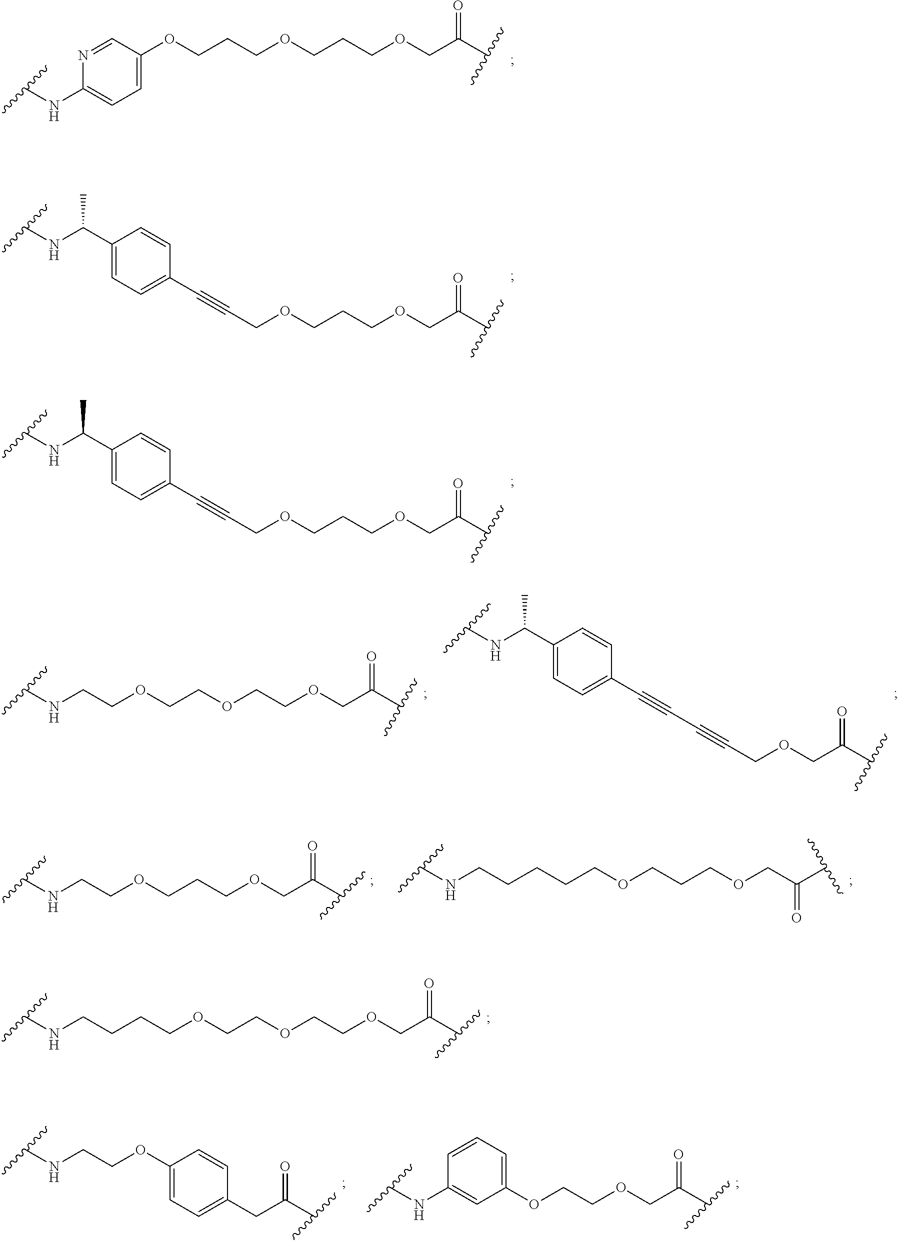 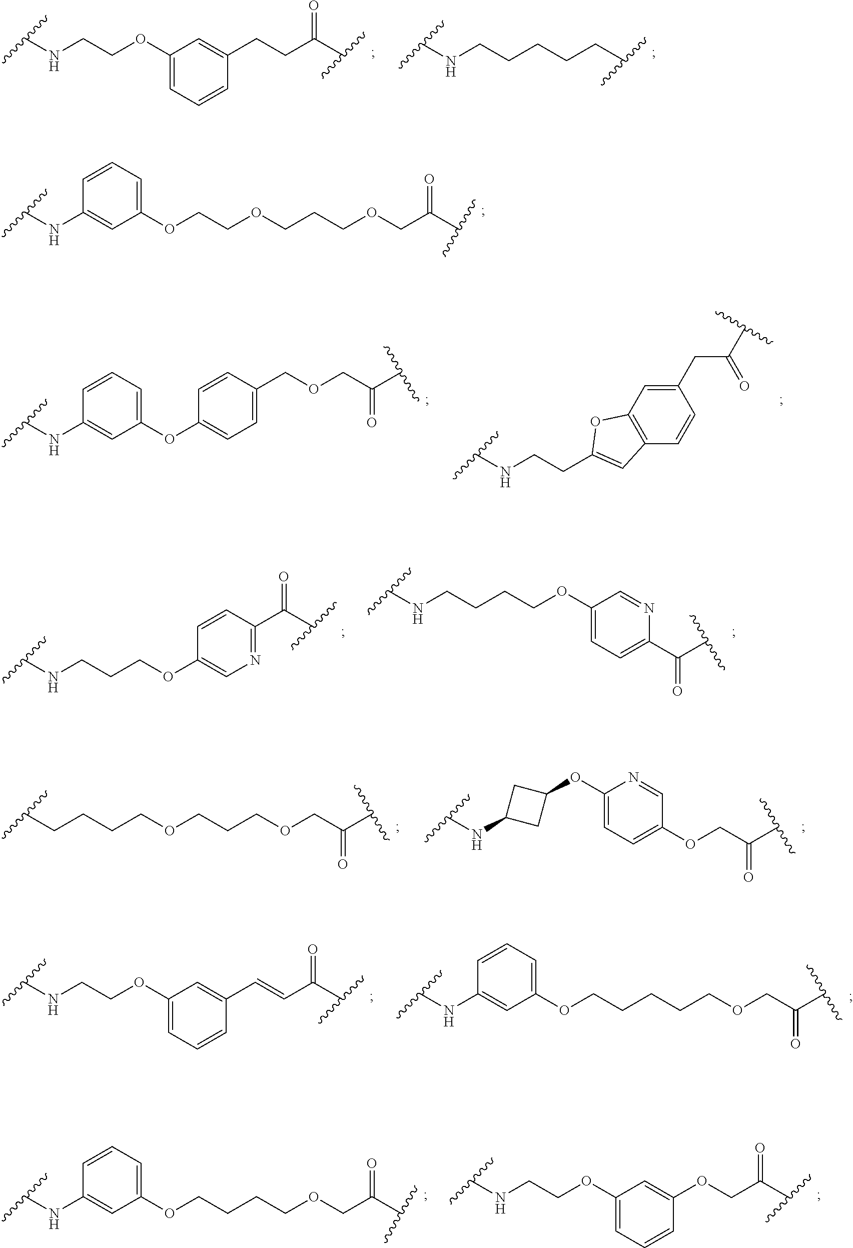 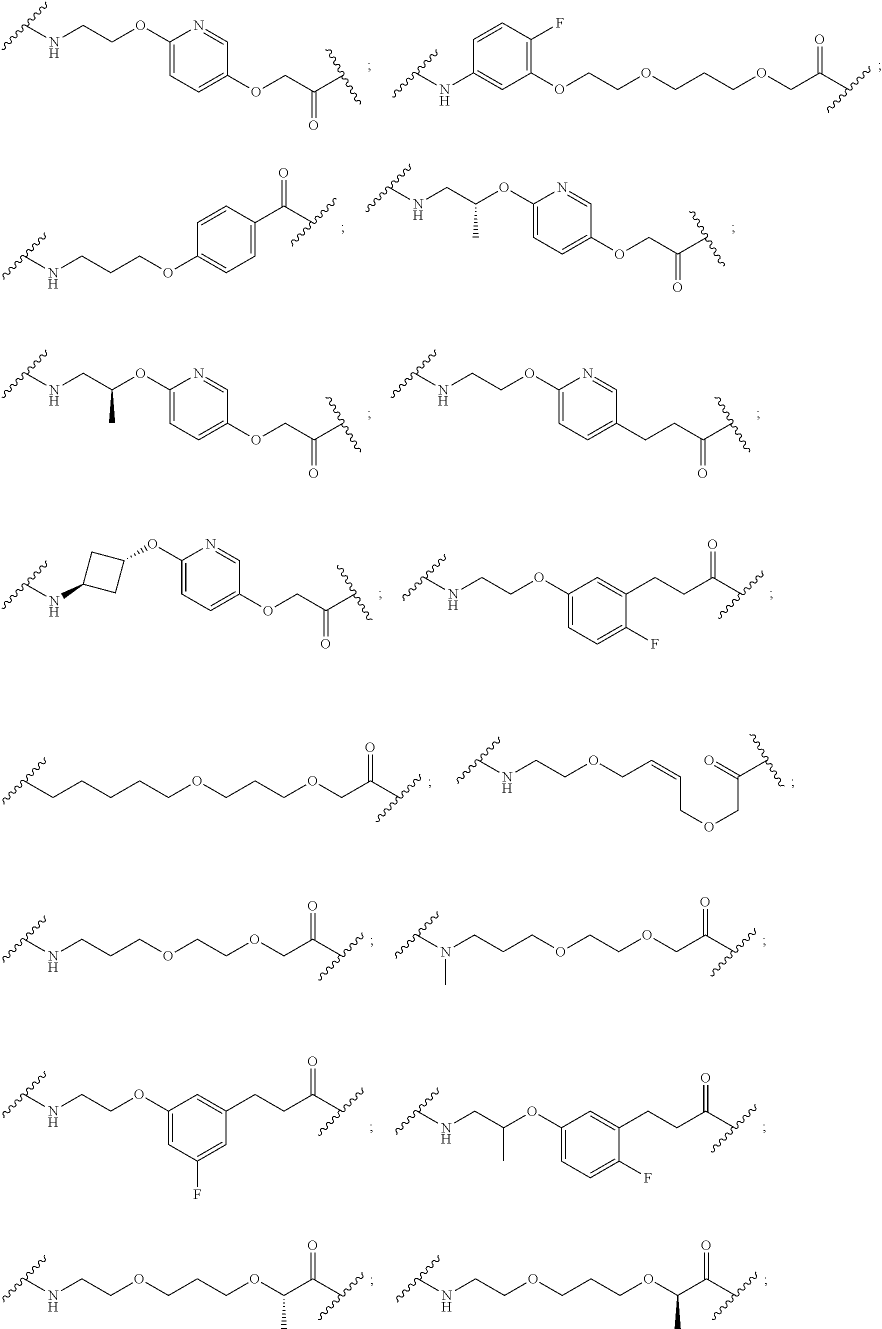 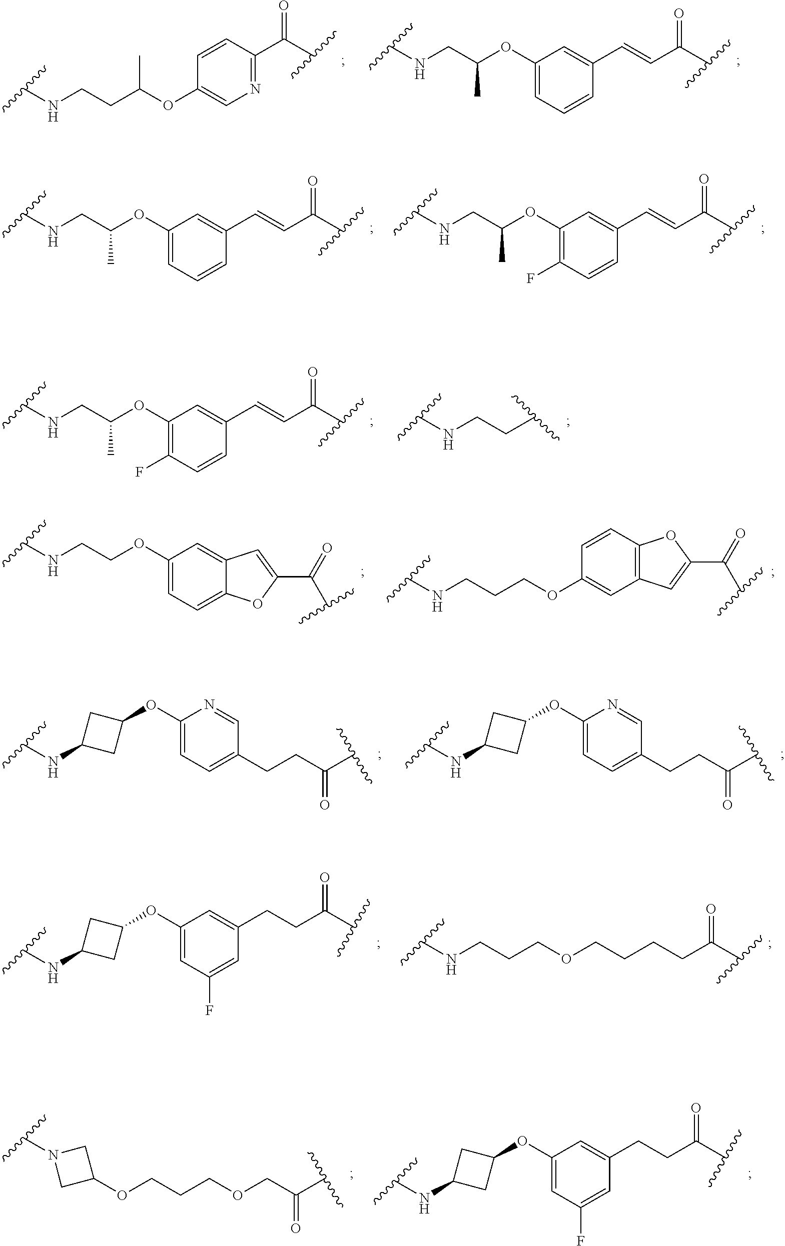 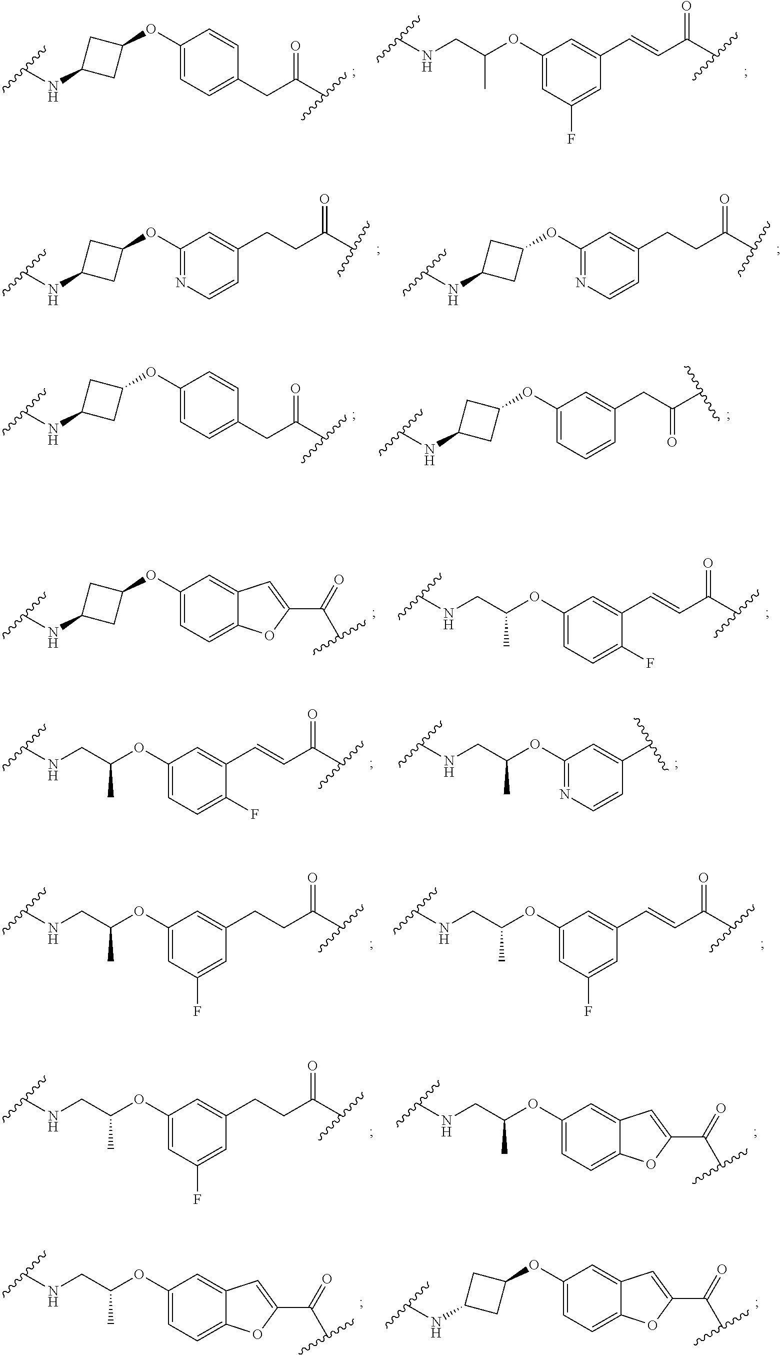 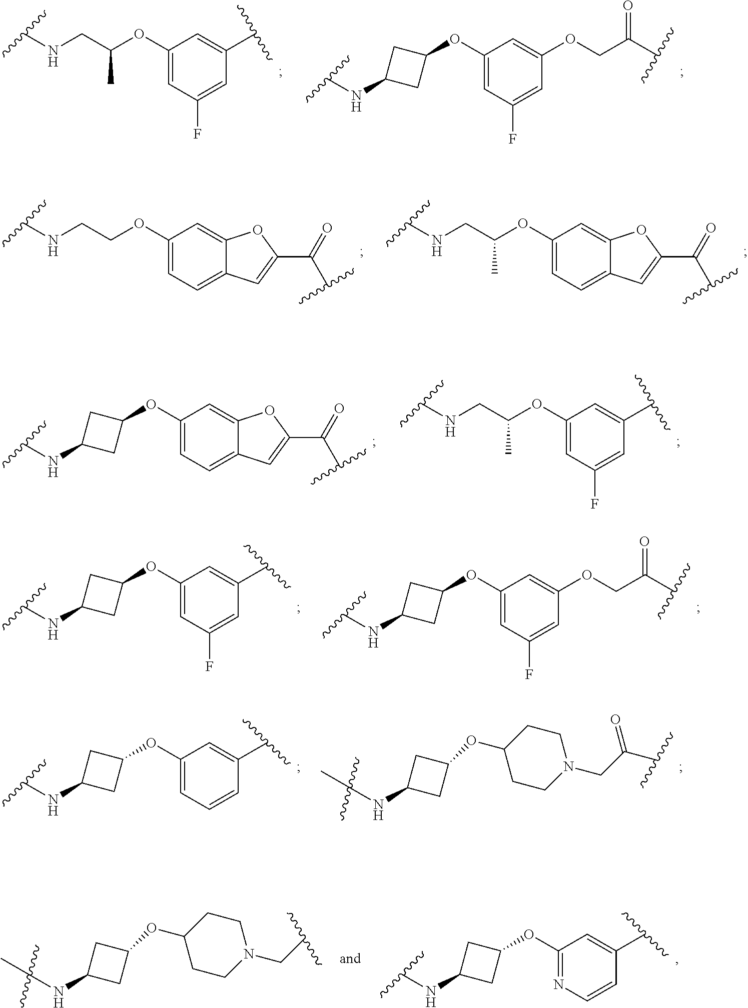
wherein each m and n is independently selected from 0, 1, 2, 3, 4, 5, 6, 7, 8, 9, 10, 11, 12, 13, 14, 15, 16, 17, 18, 19, or 20.

18. The compound according to claim 15, wherein the unit A^{L} of linker (L) is selected from the group consisting of: 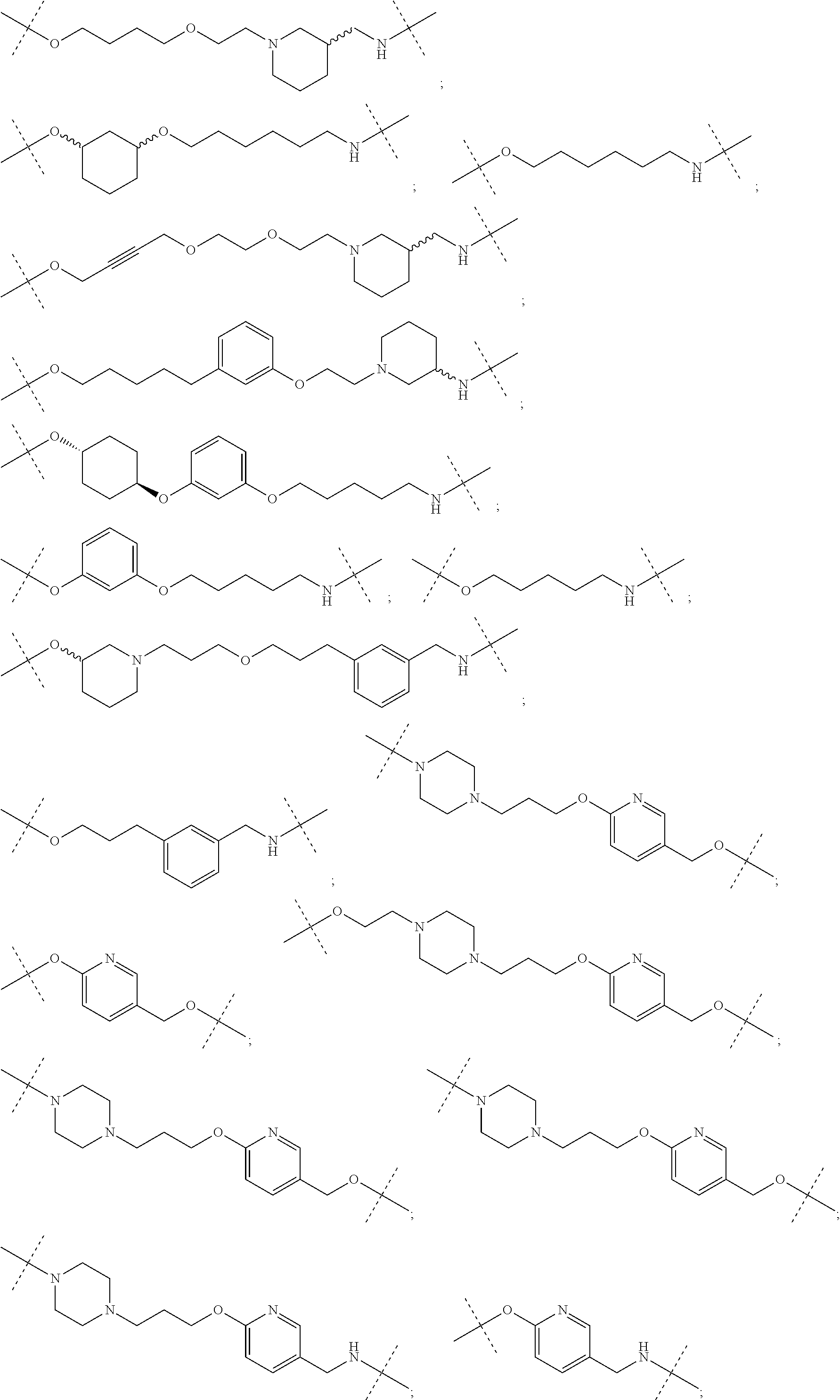 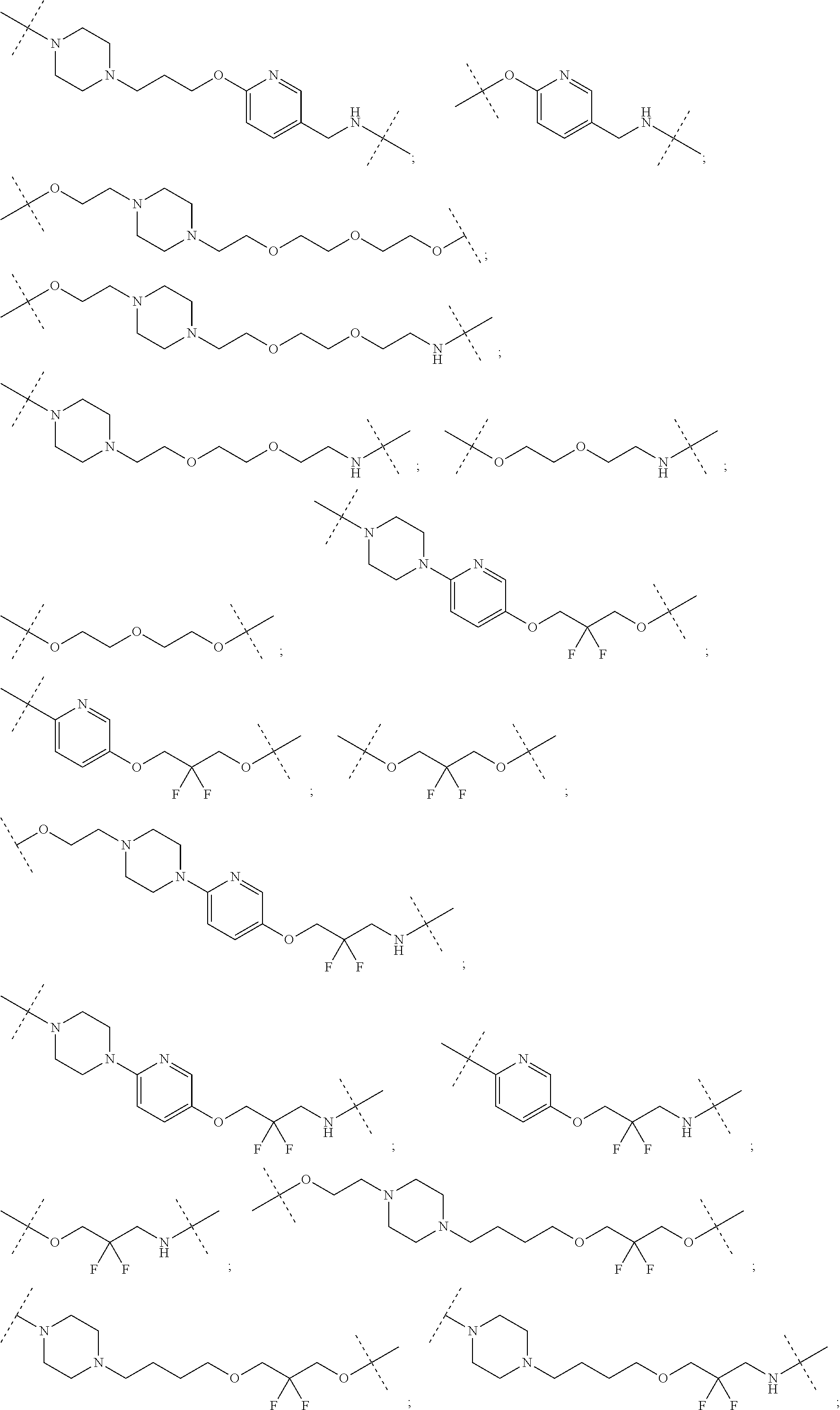 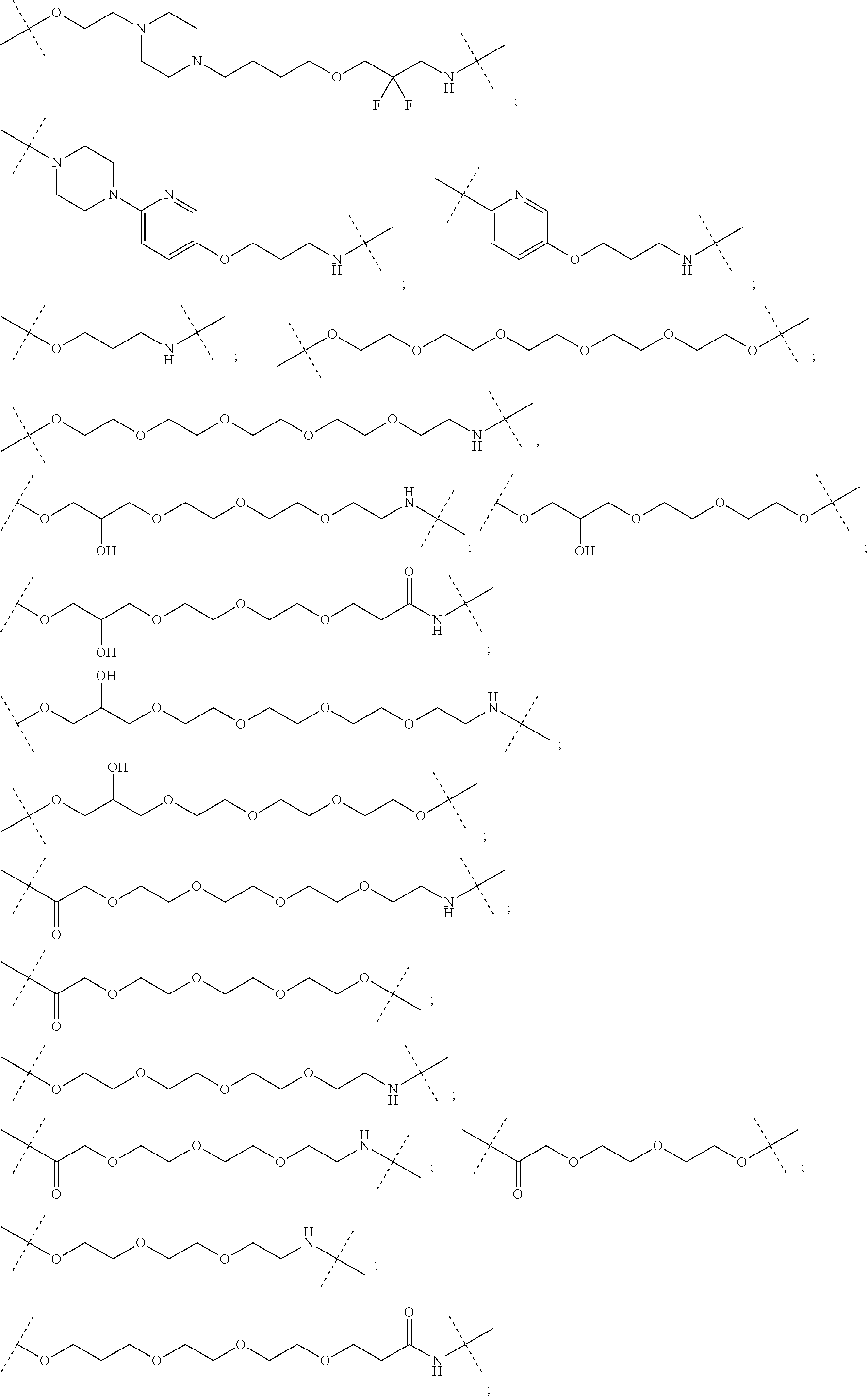 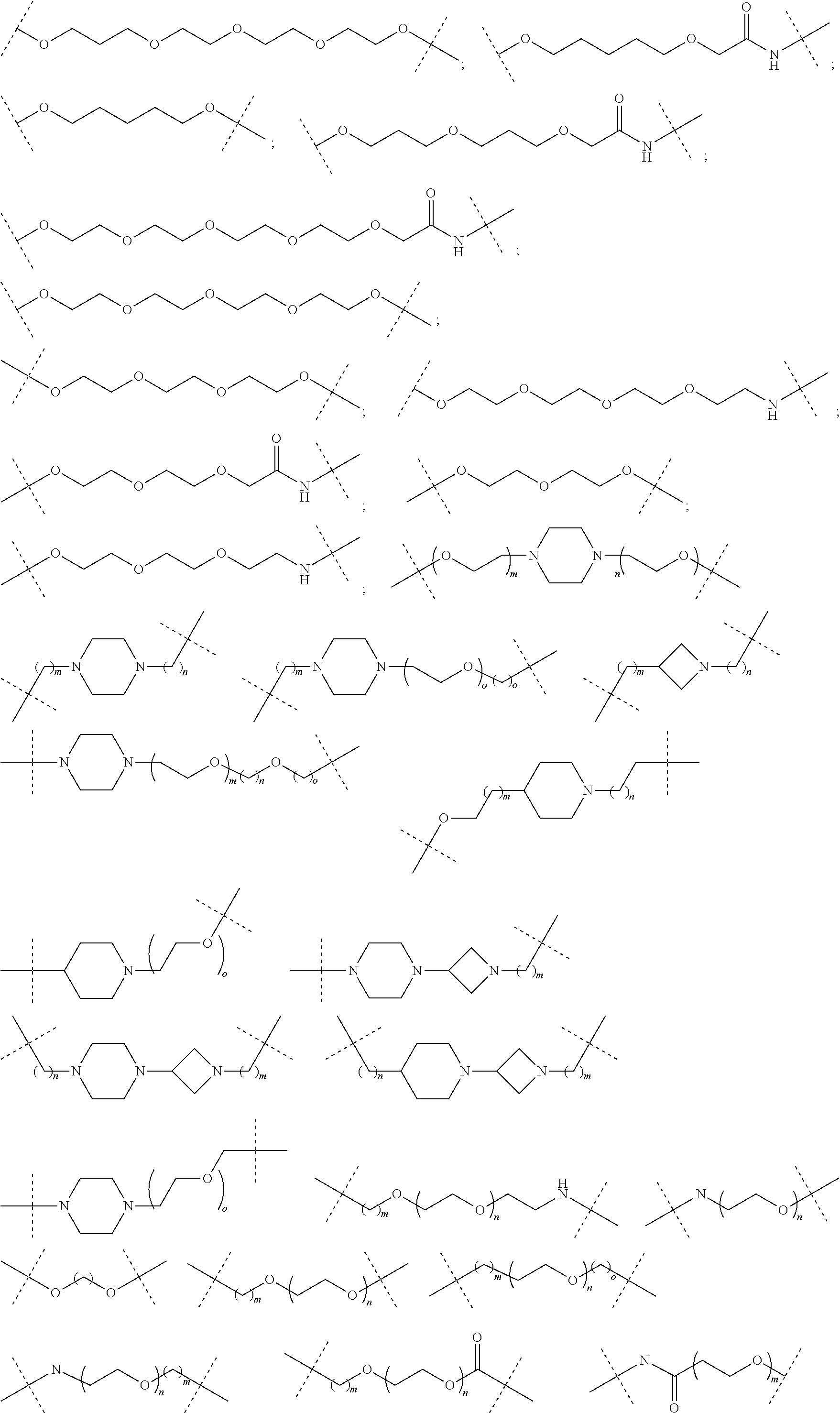 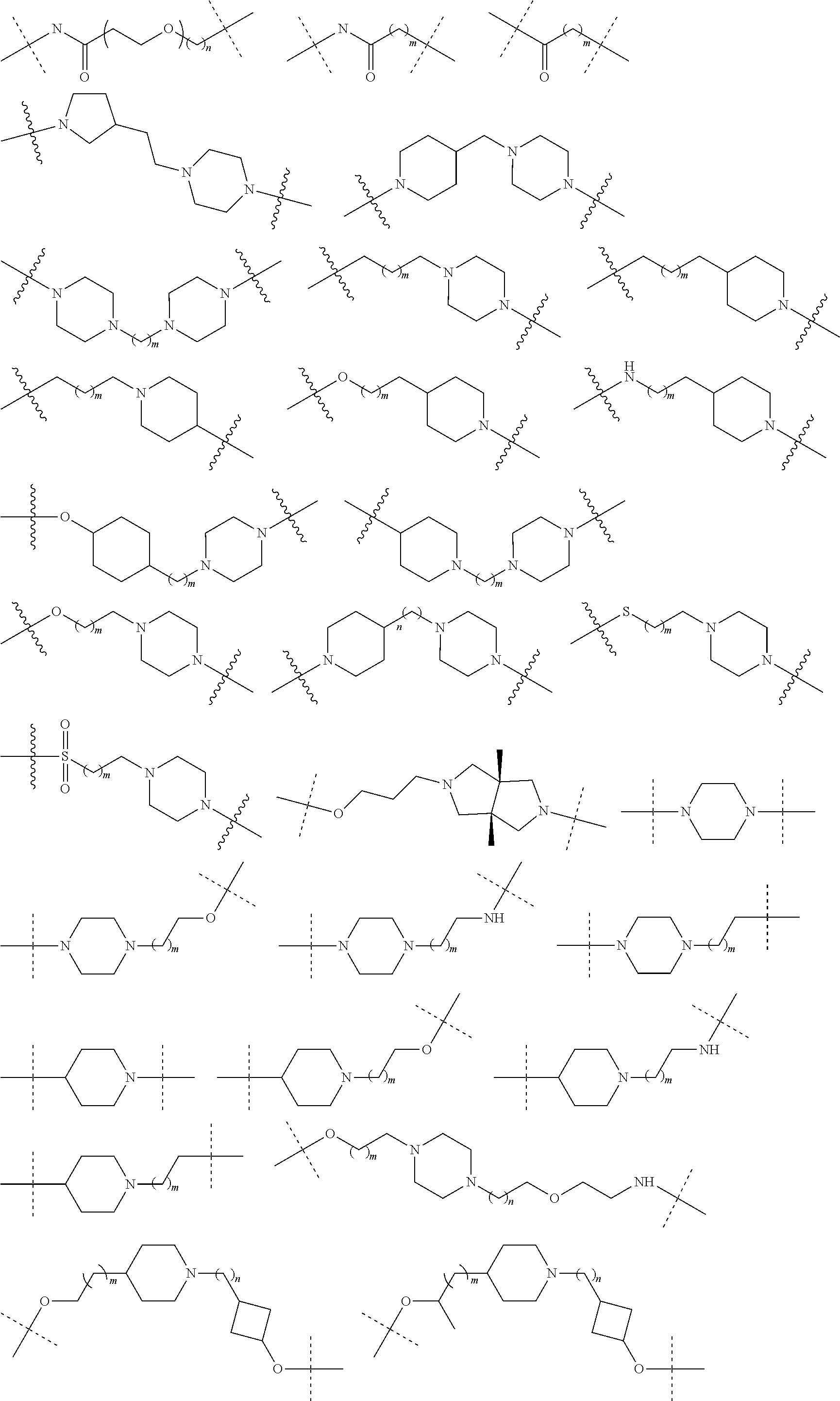 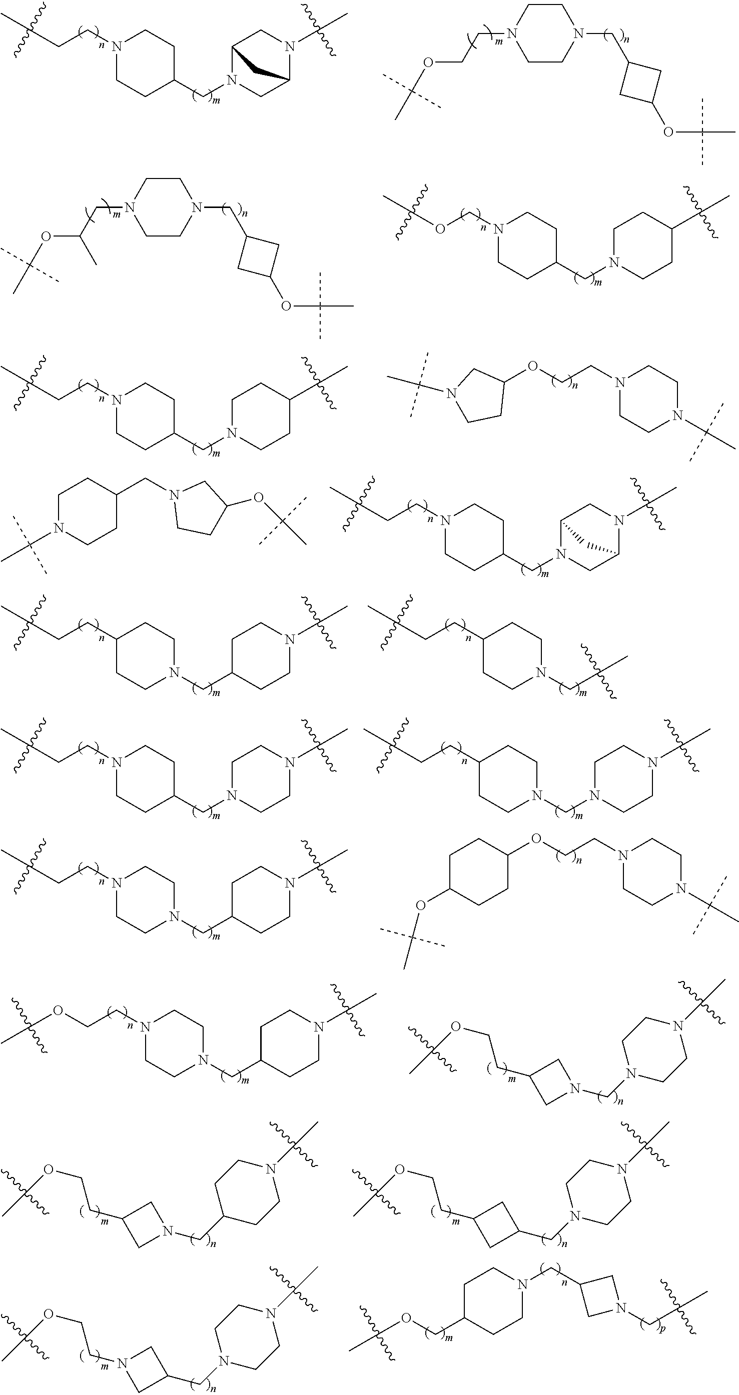 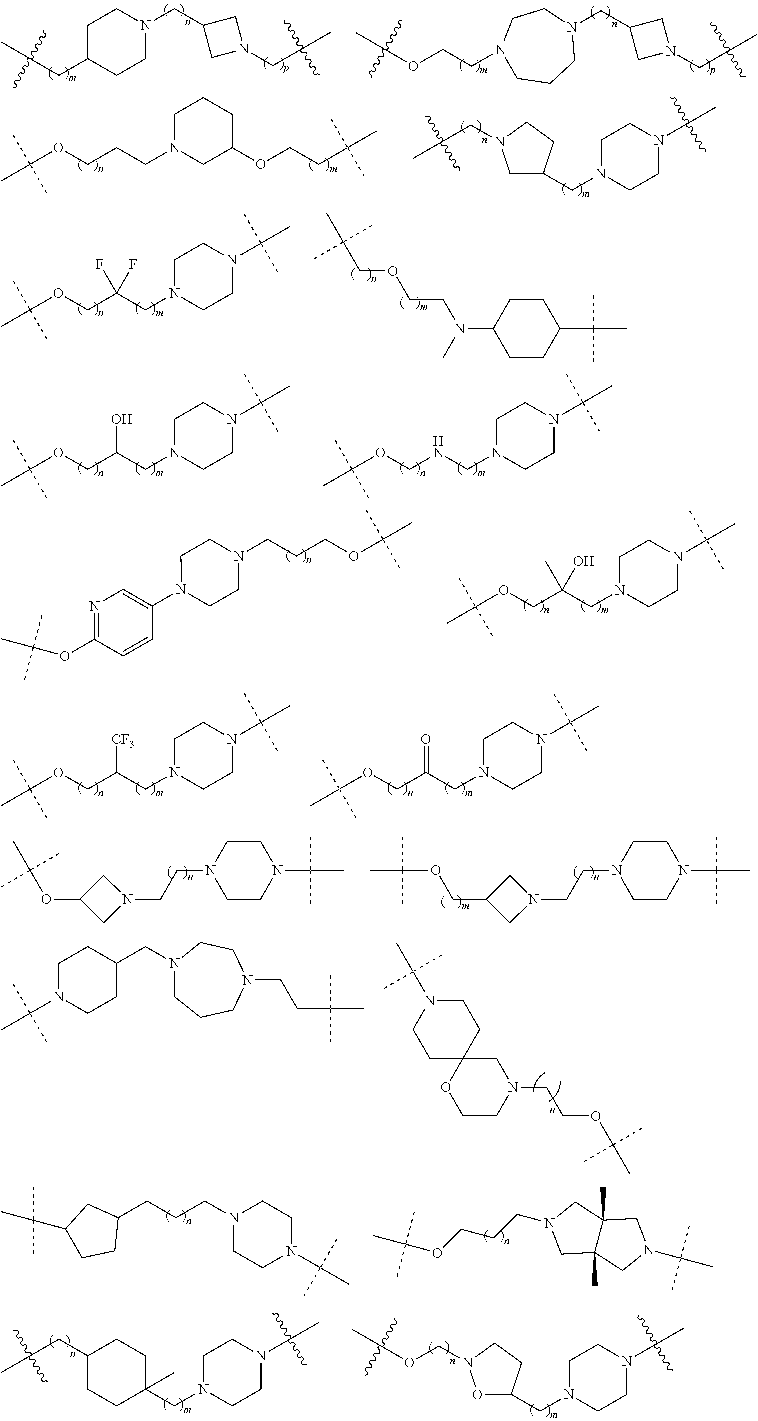 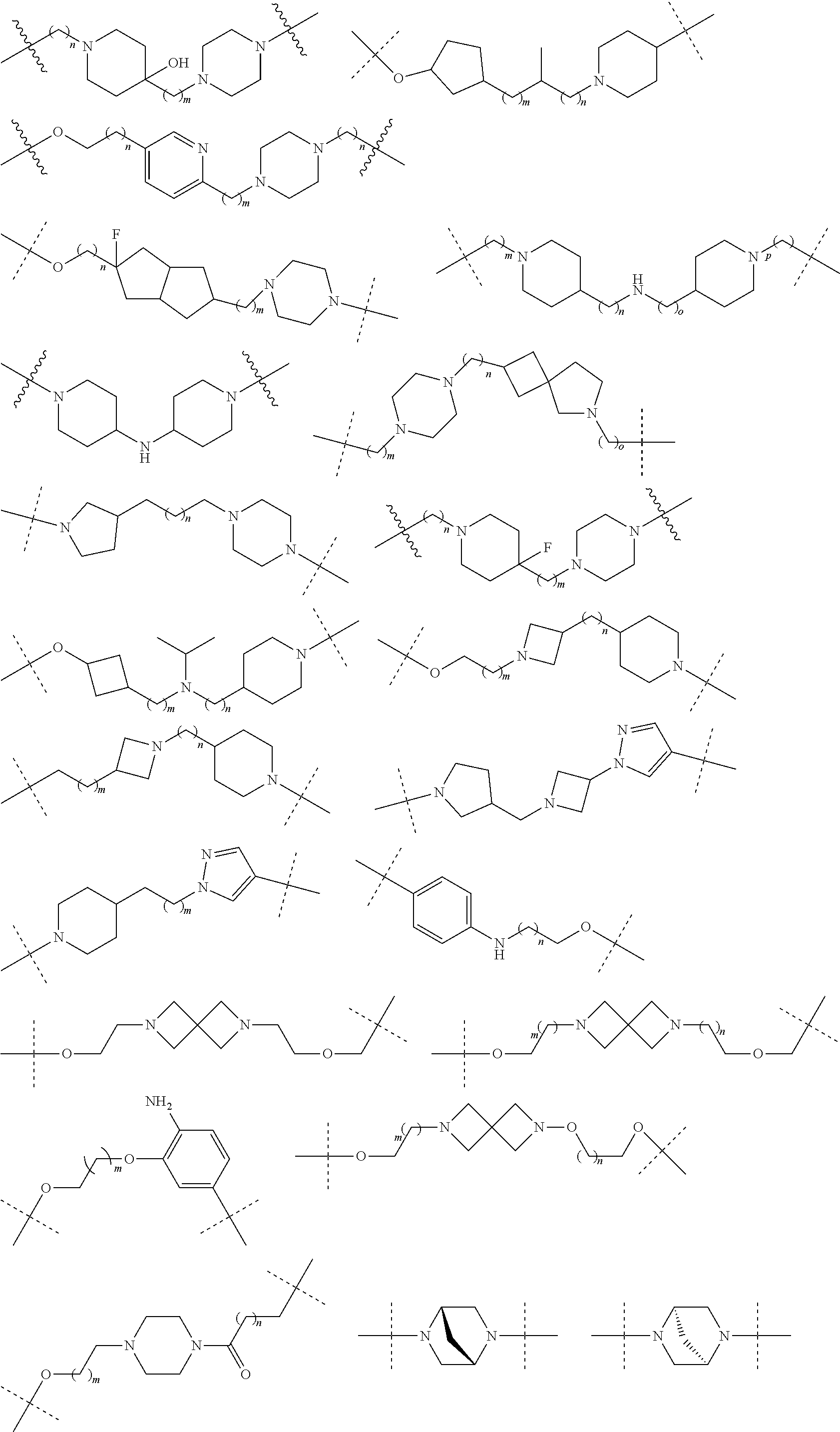 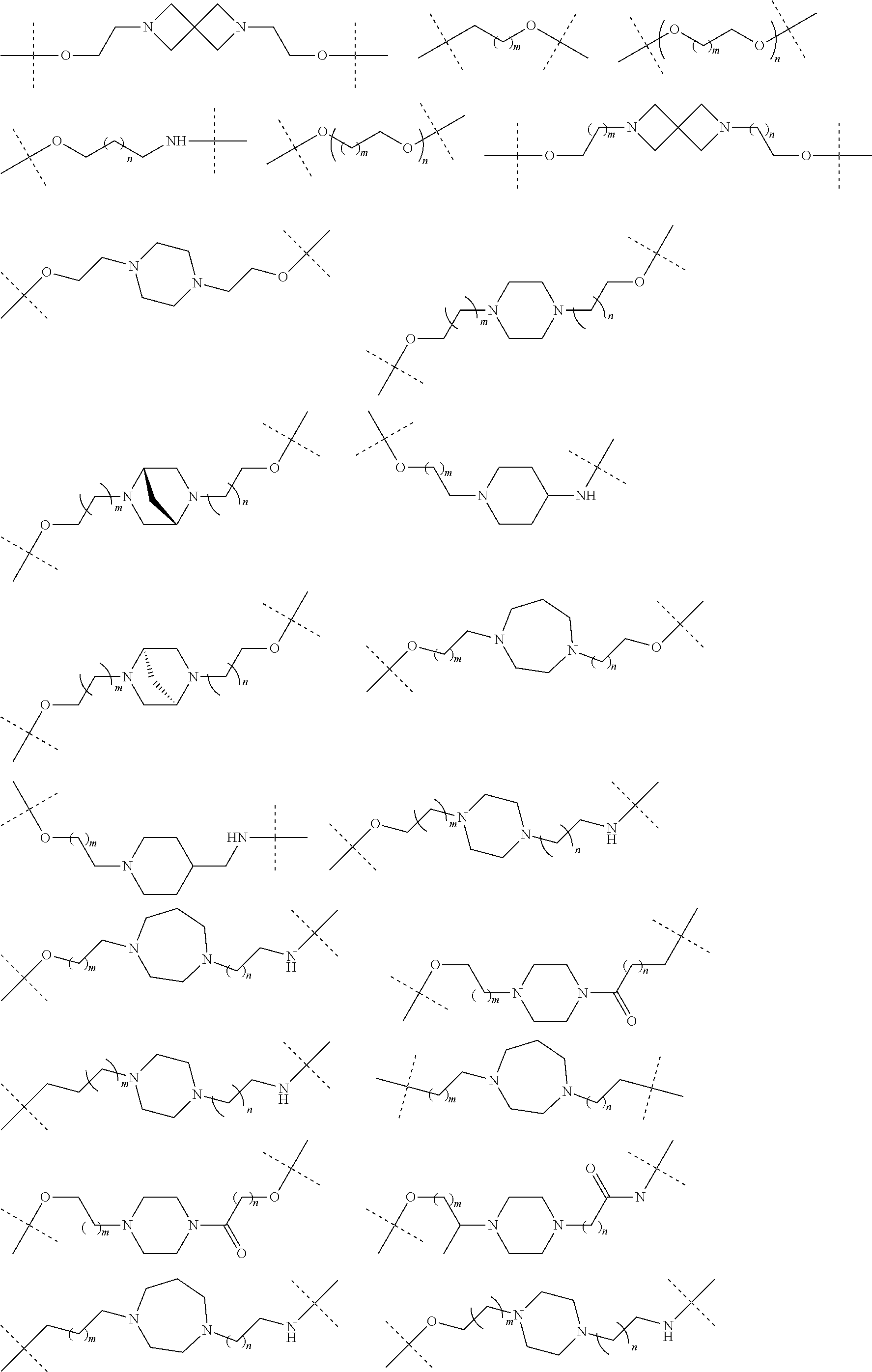 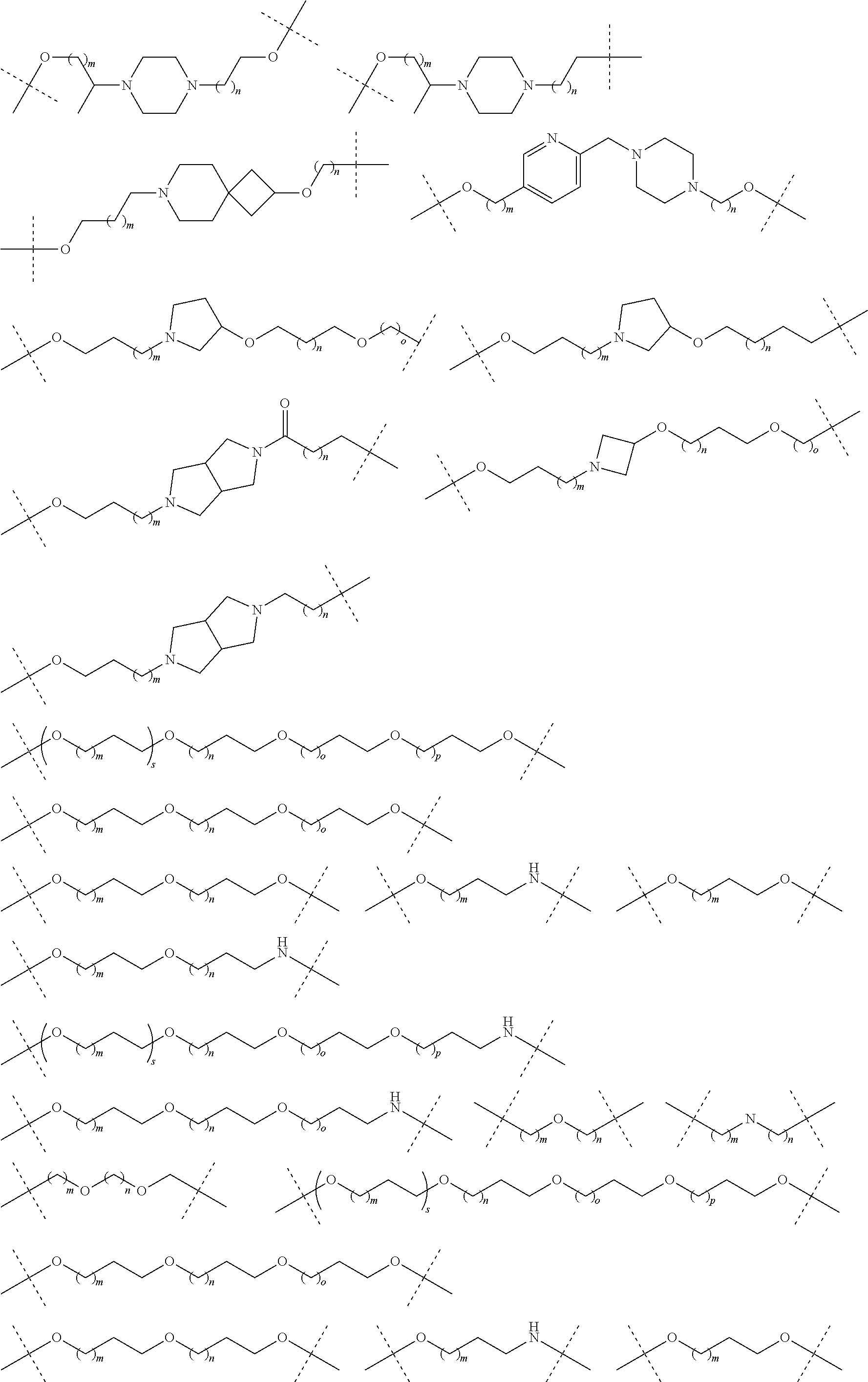 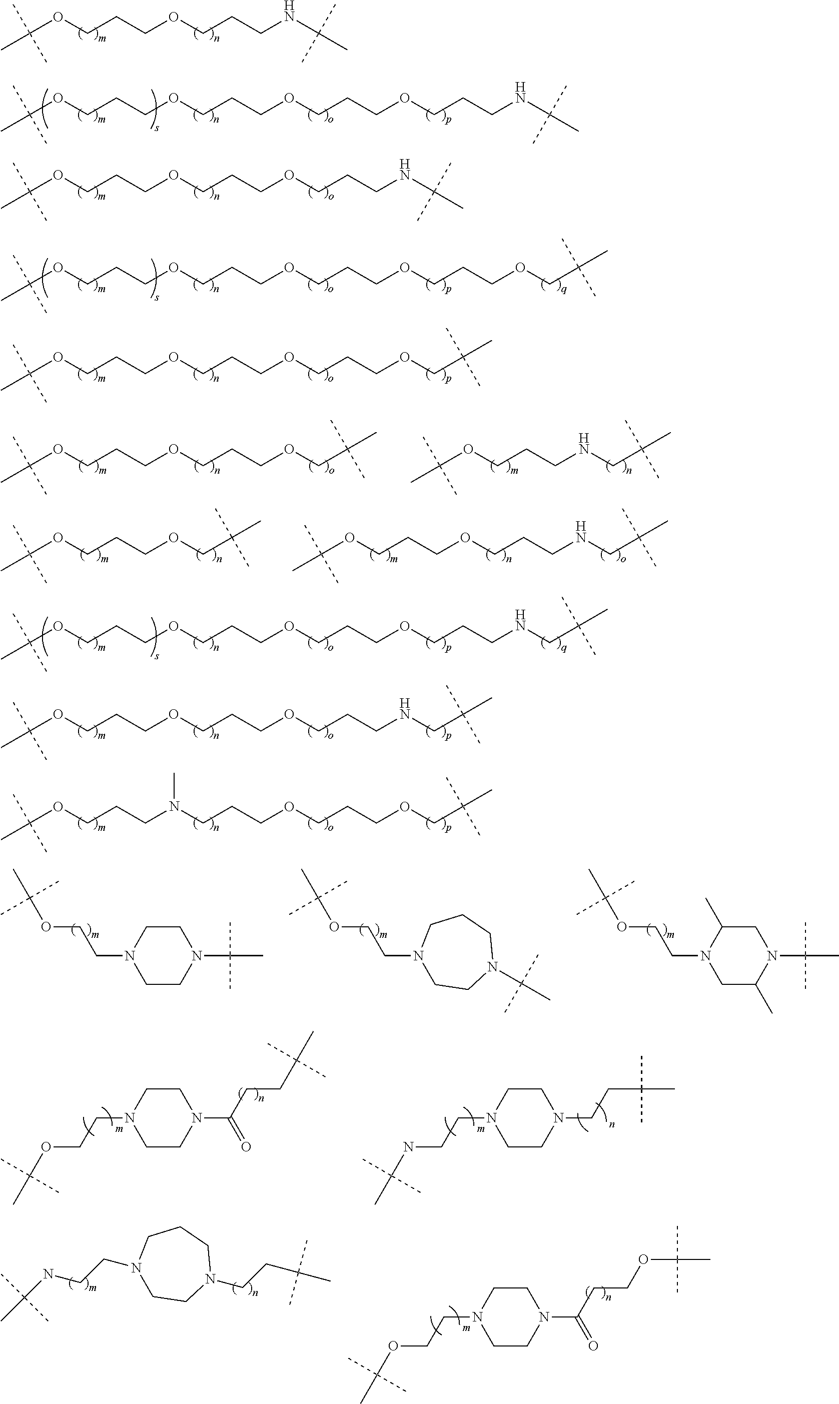 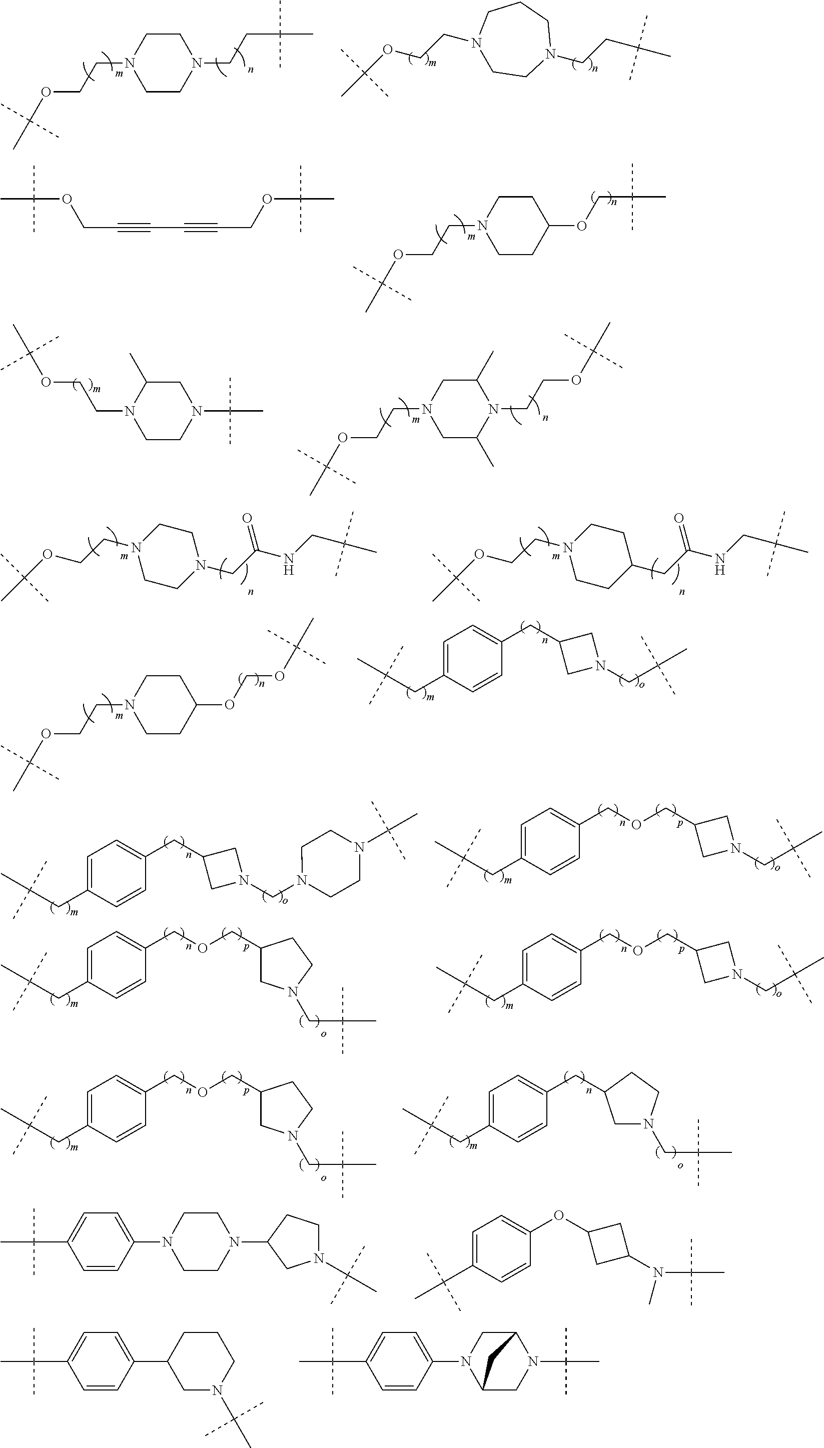 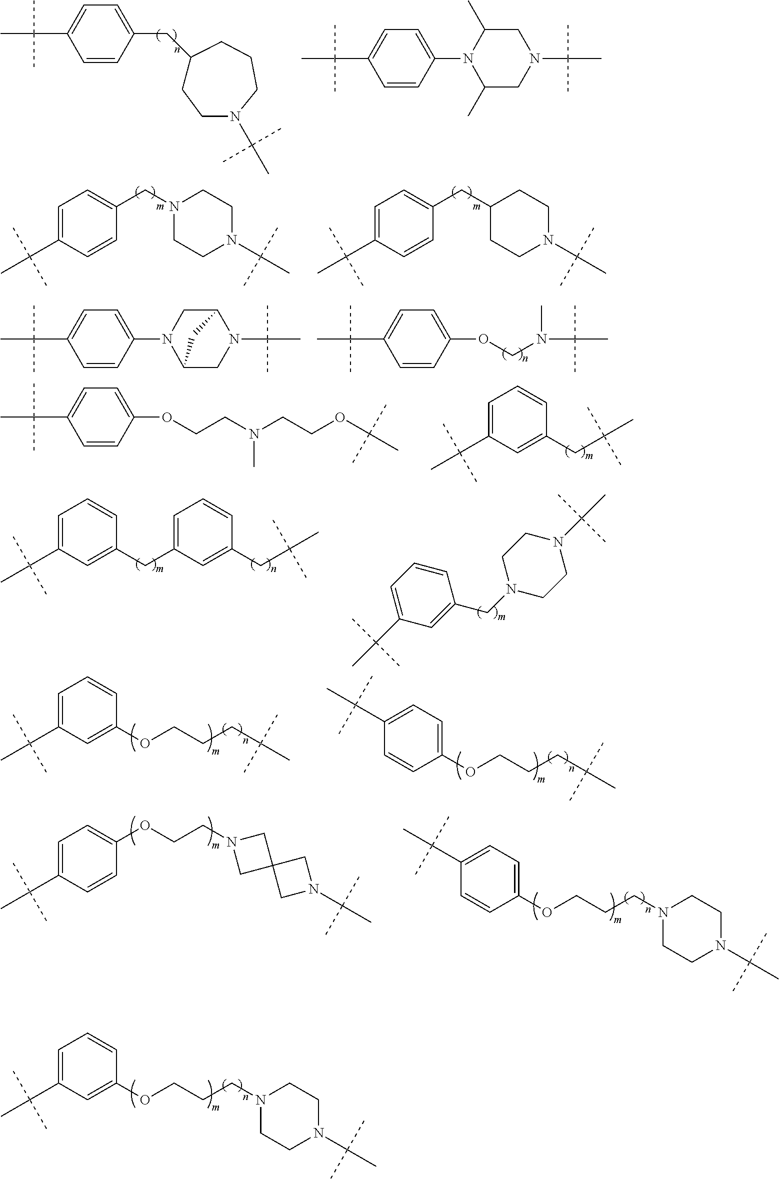 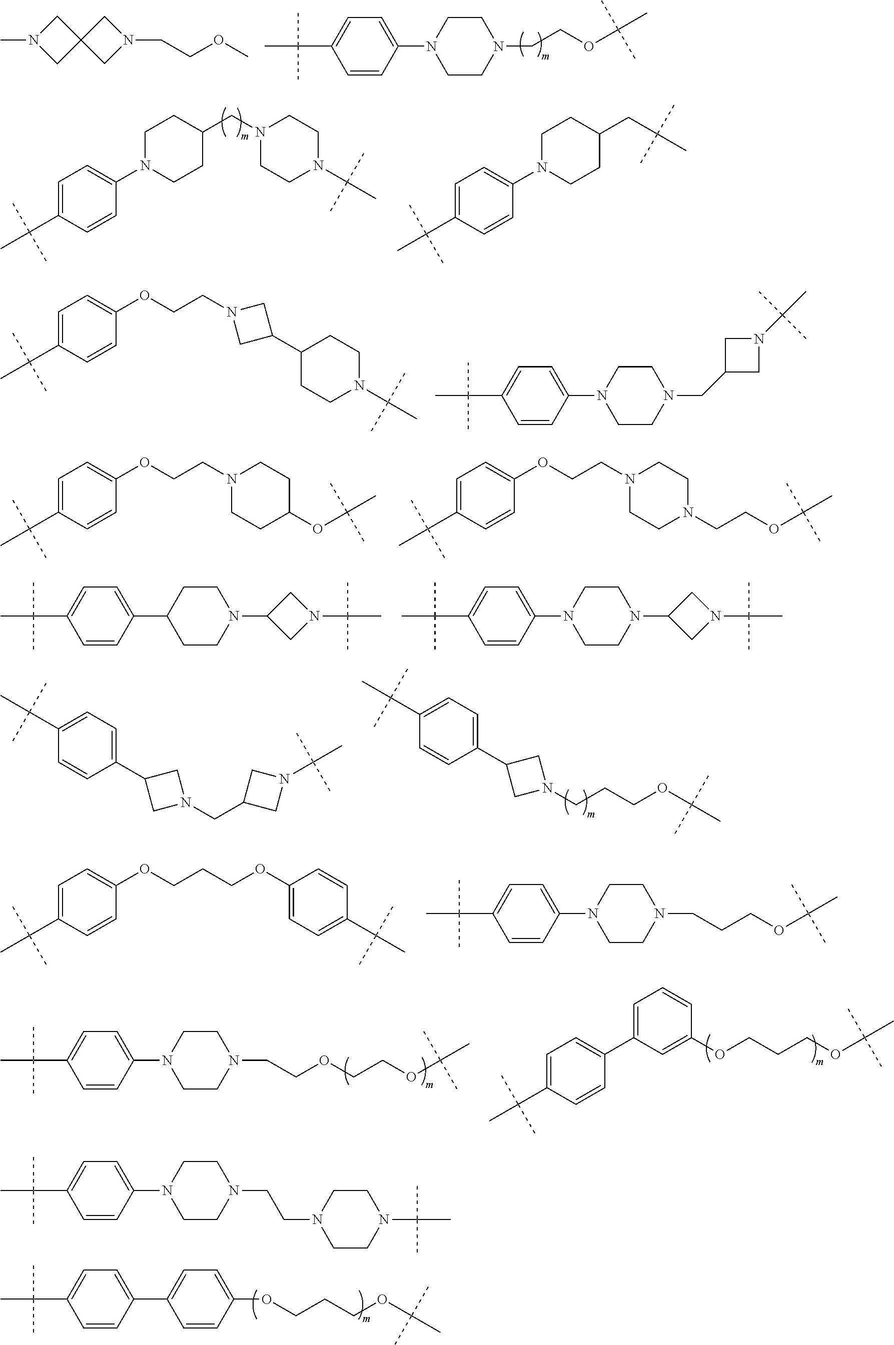 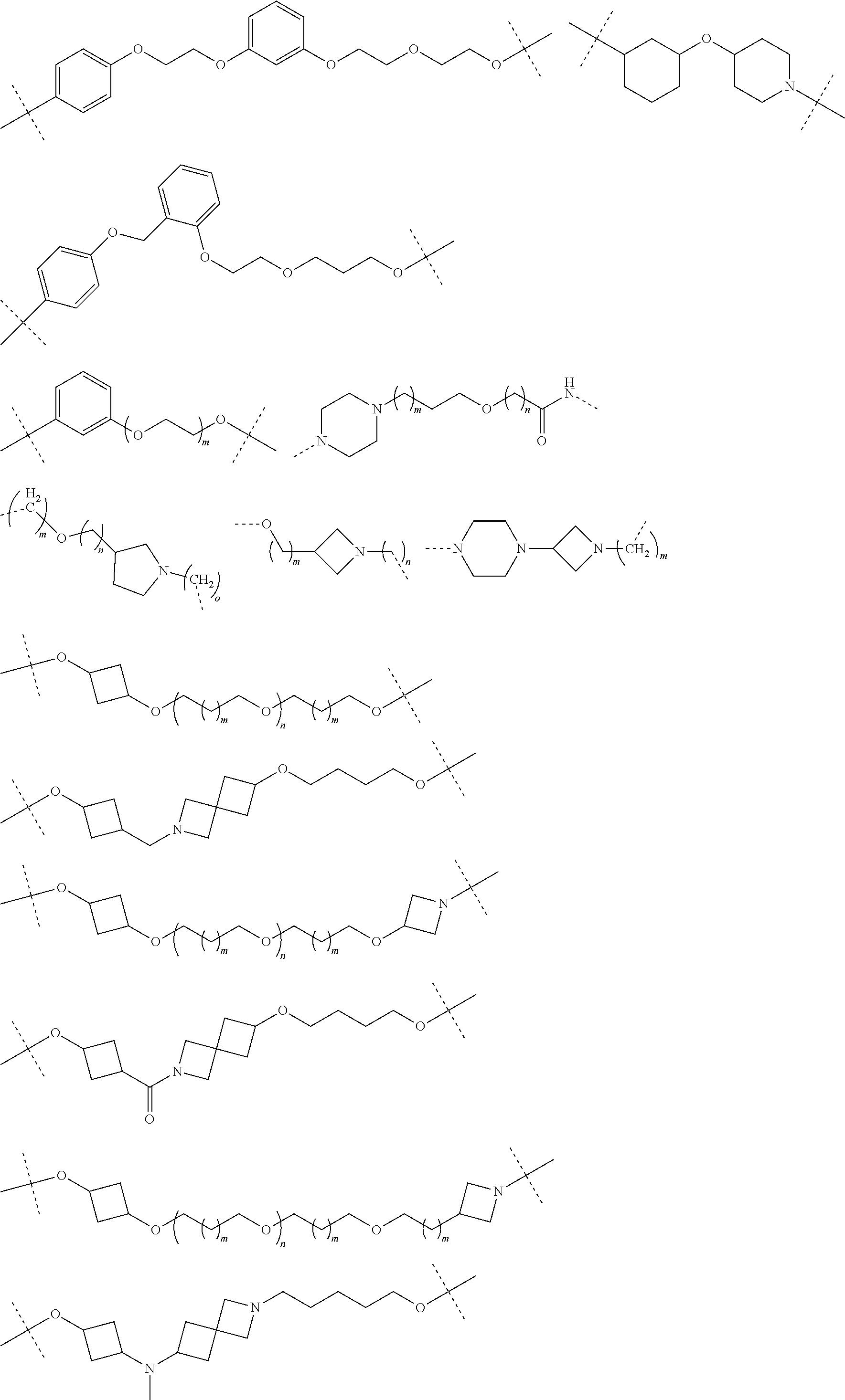 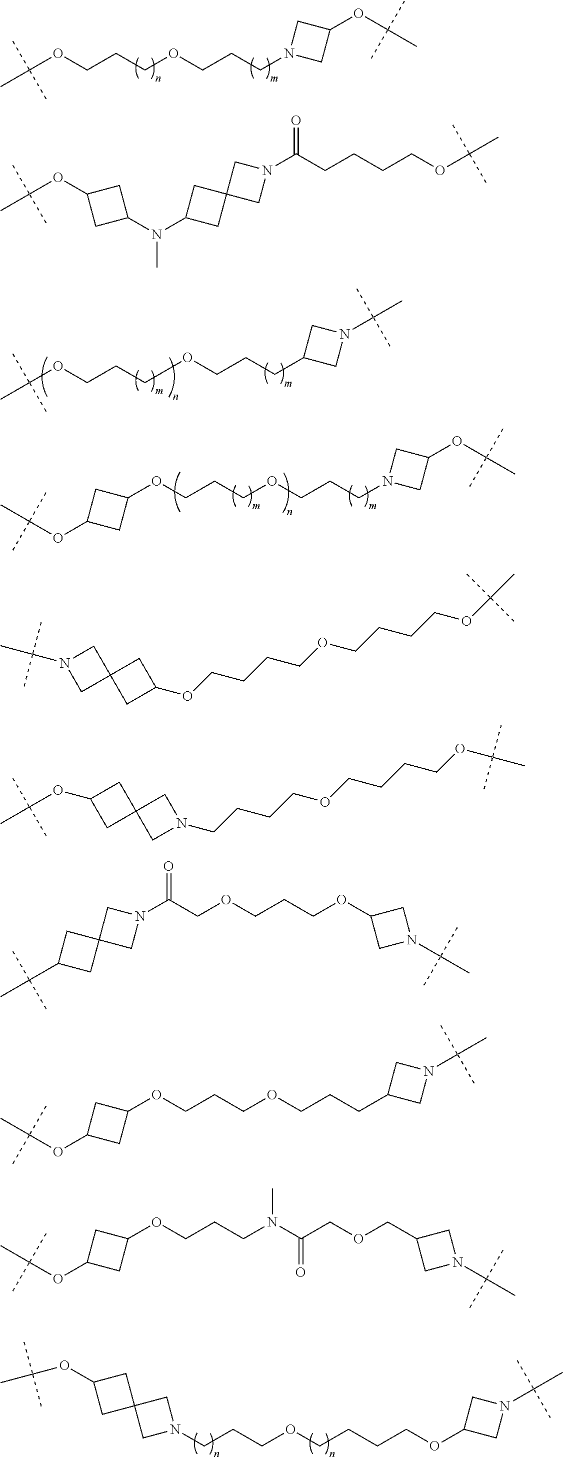 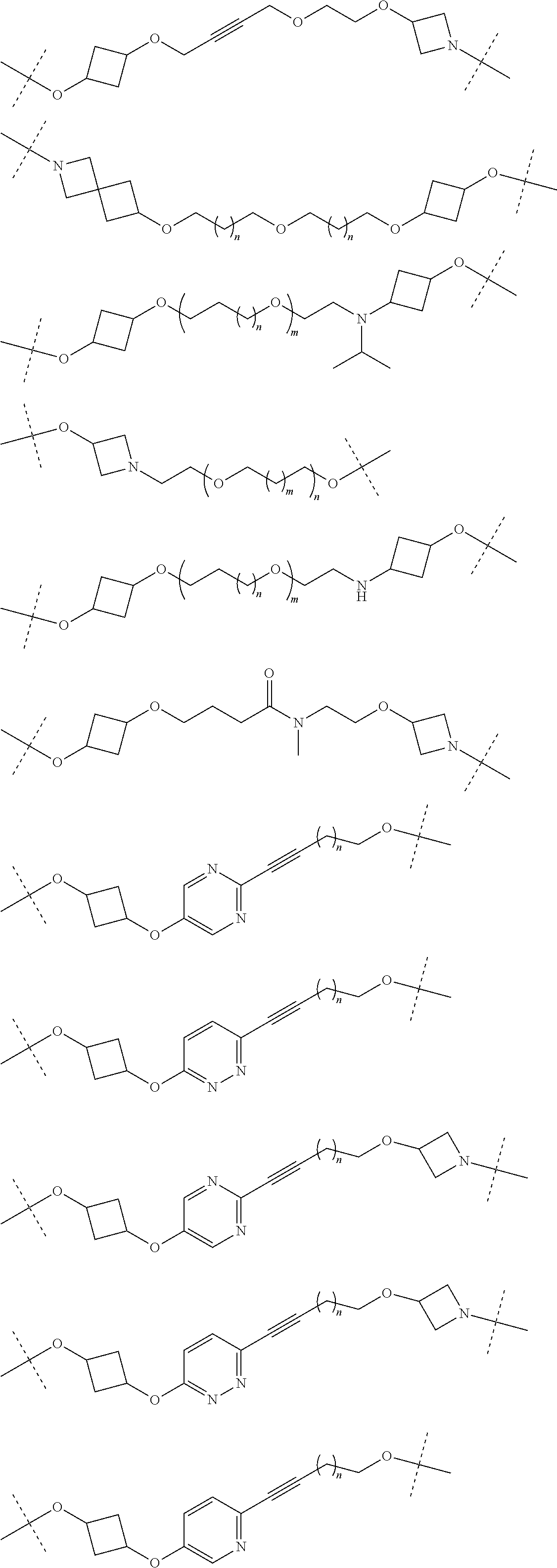 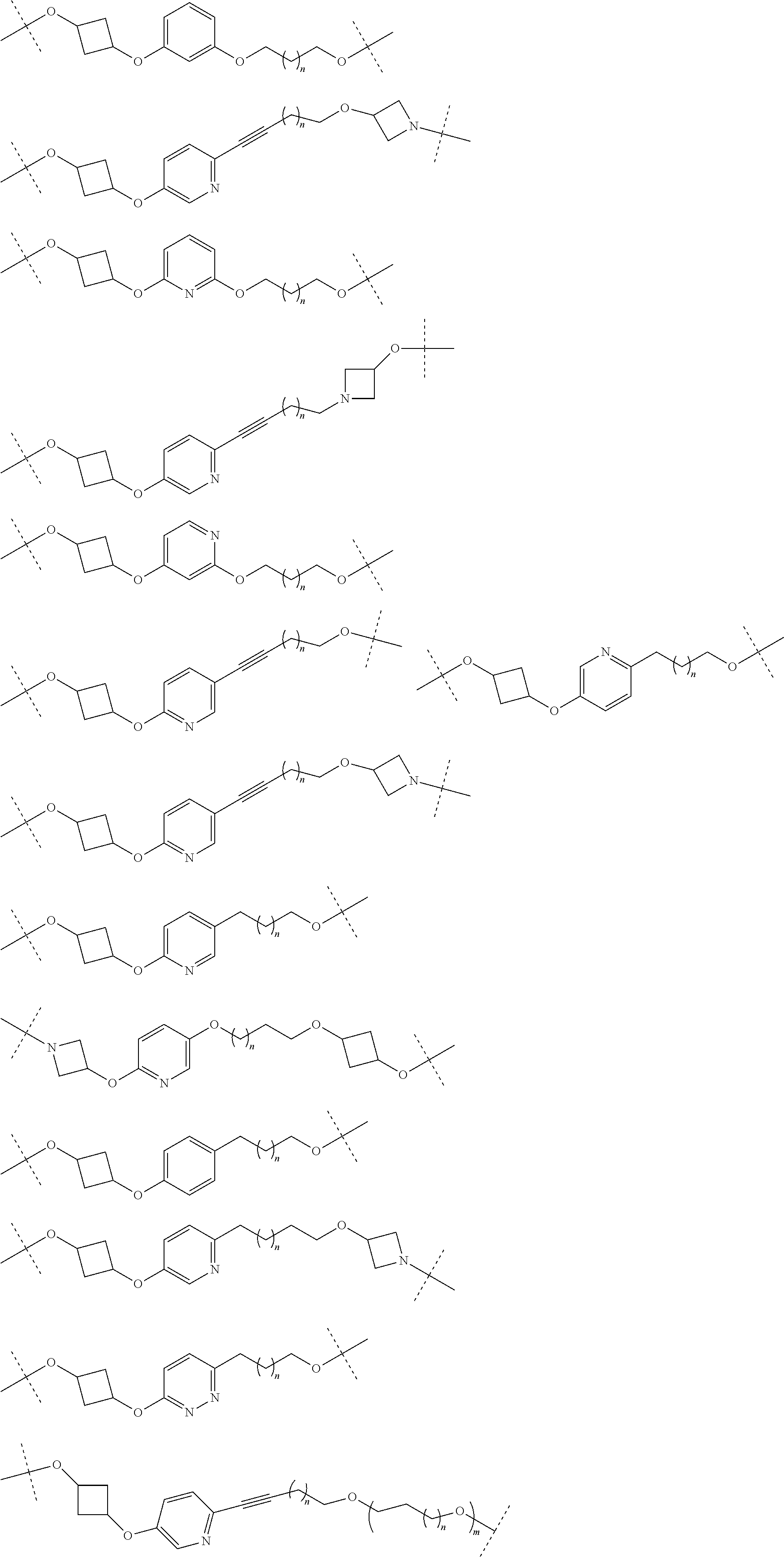 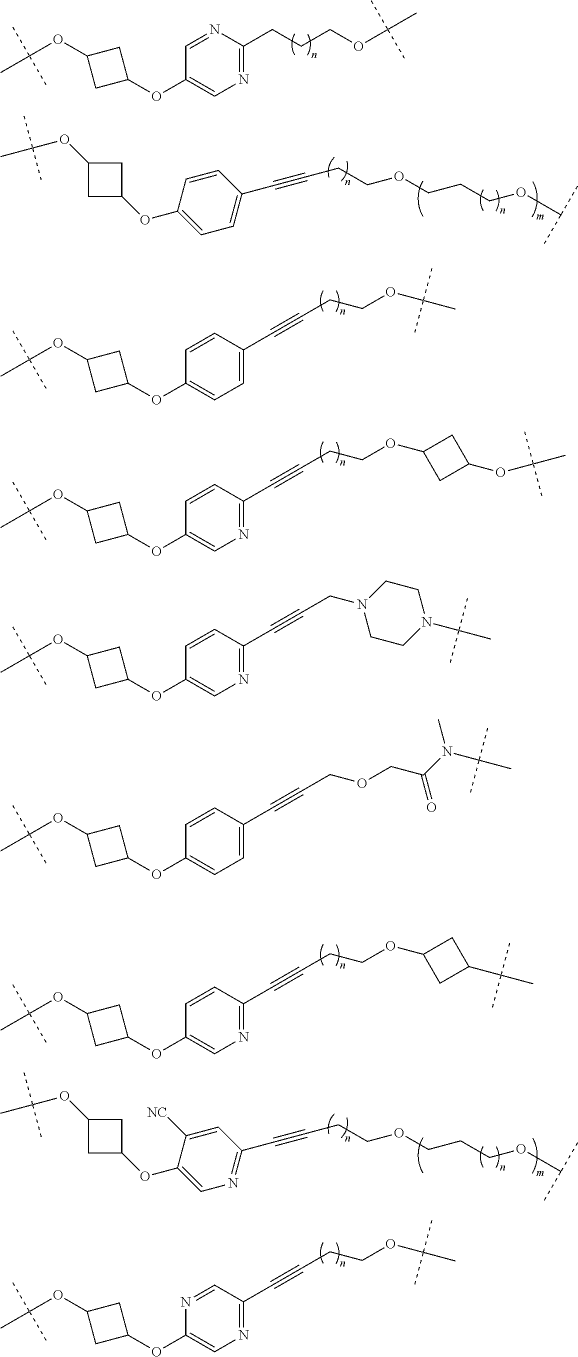 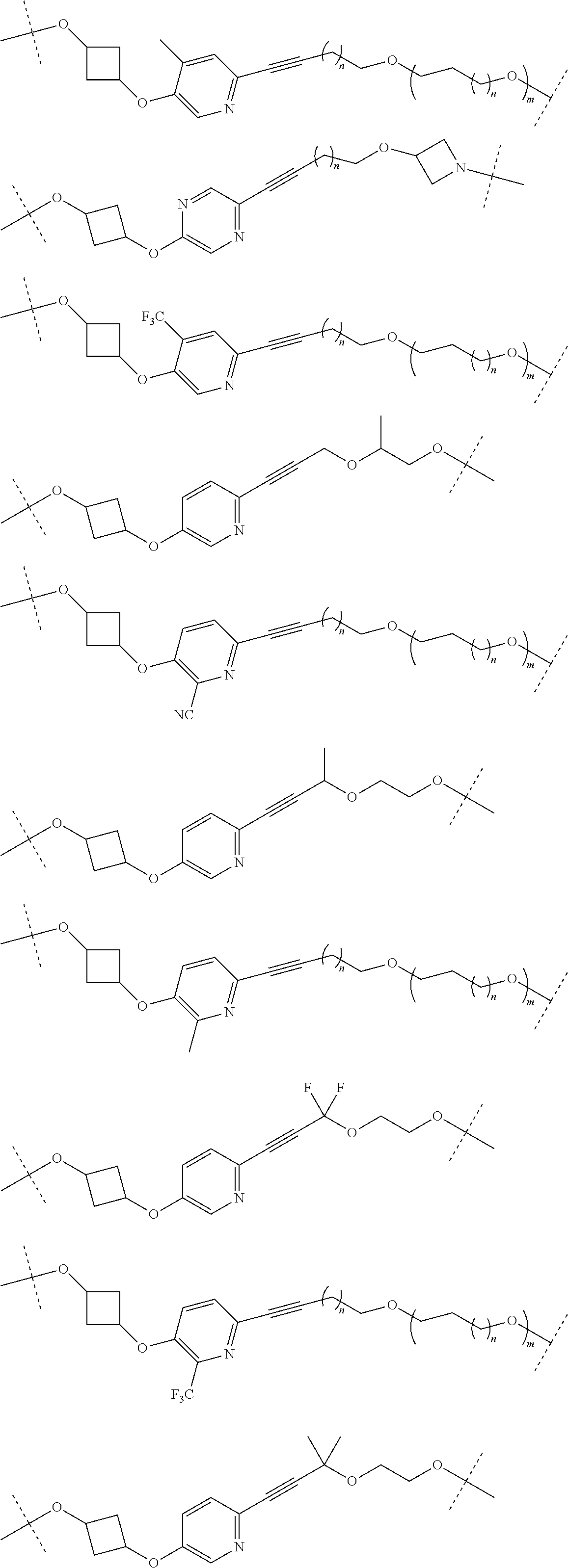 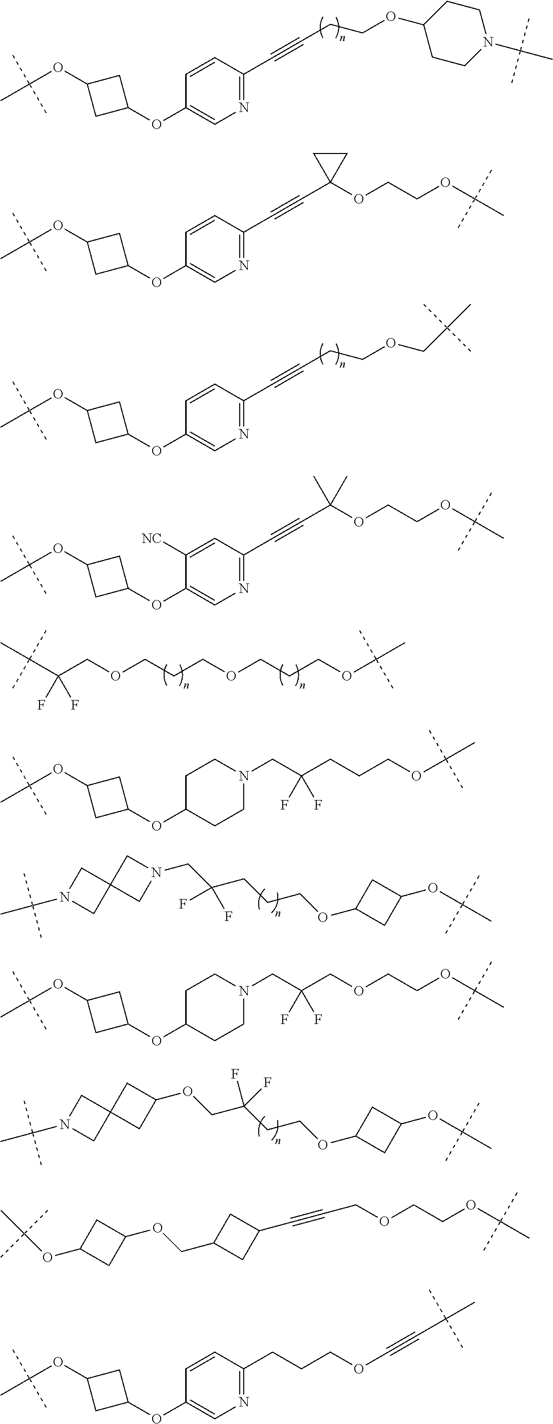 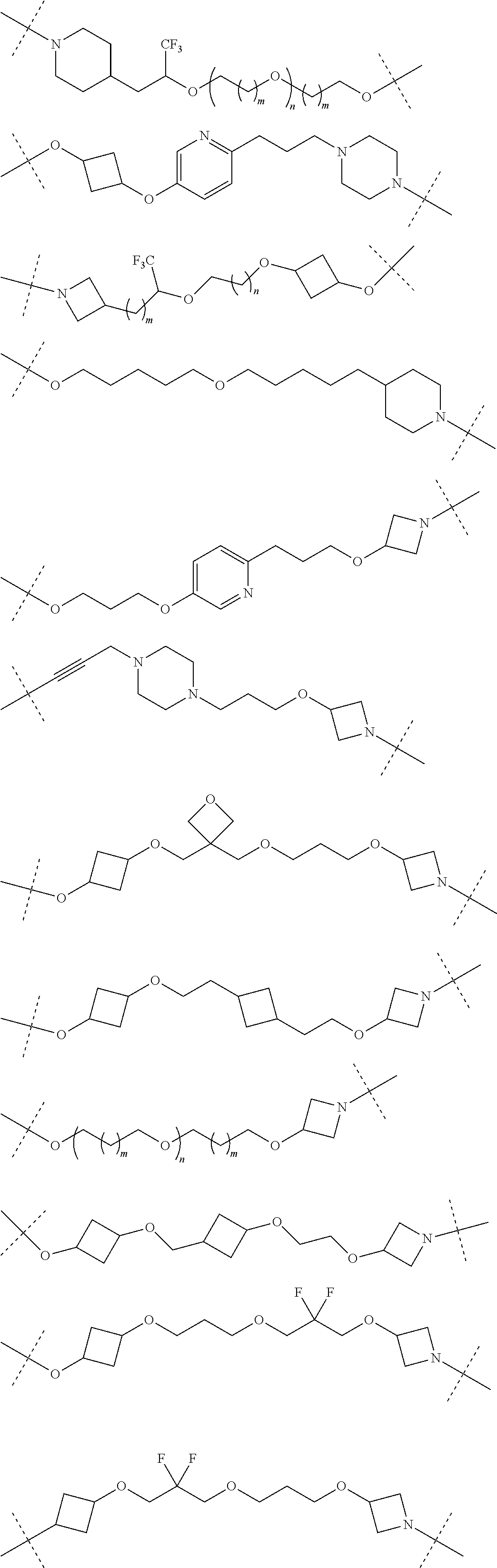 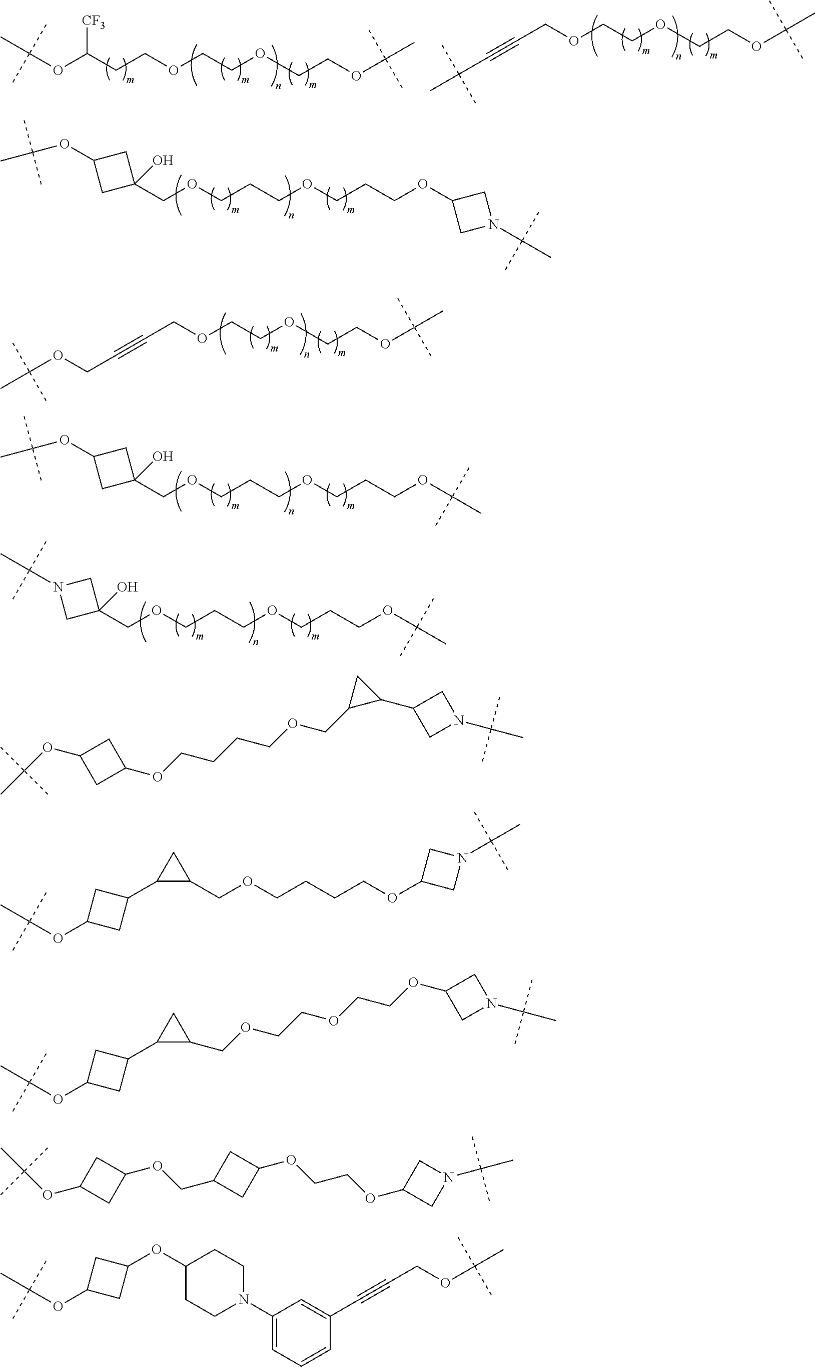 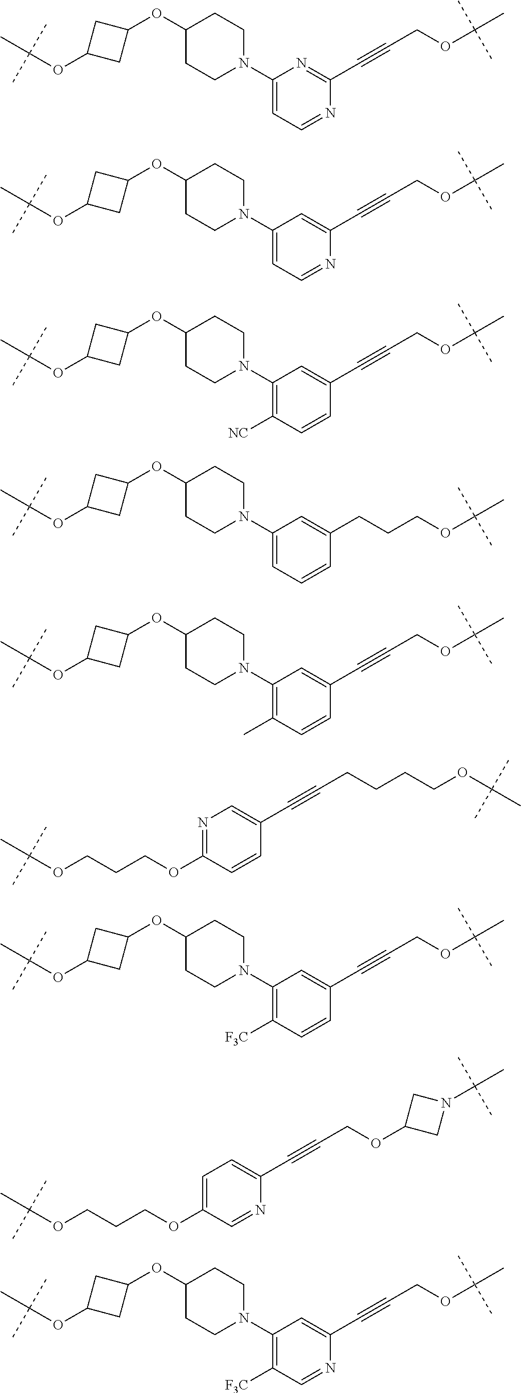 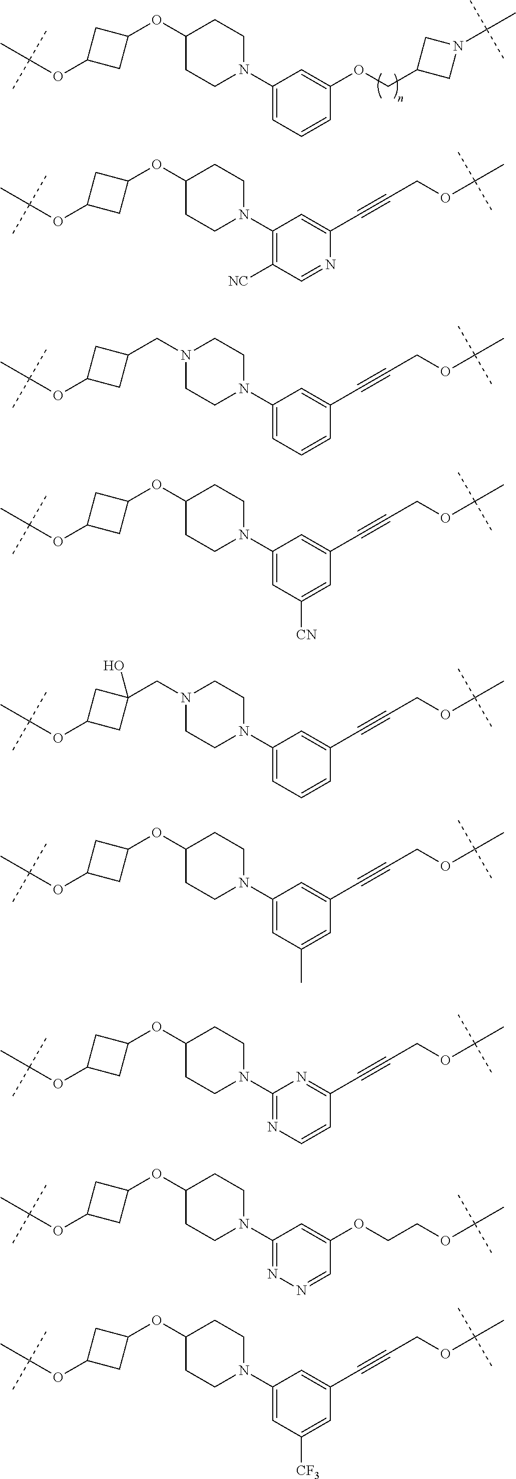 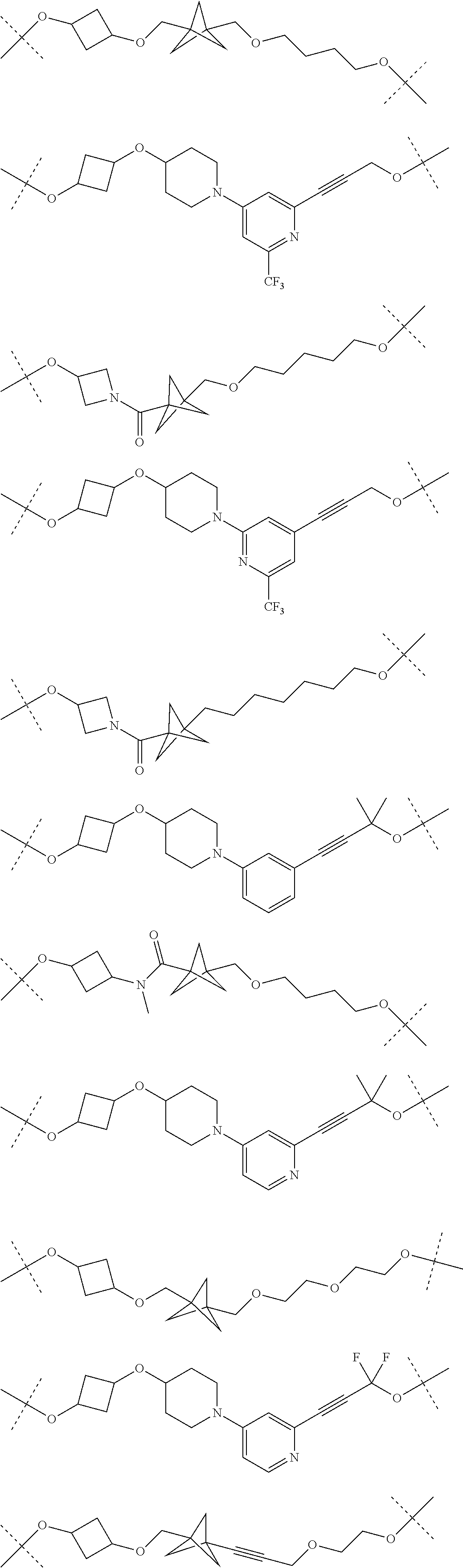 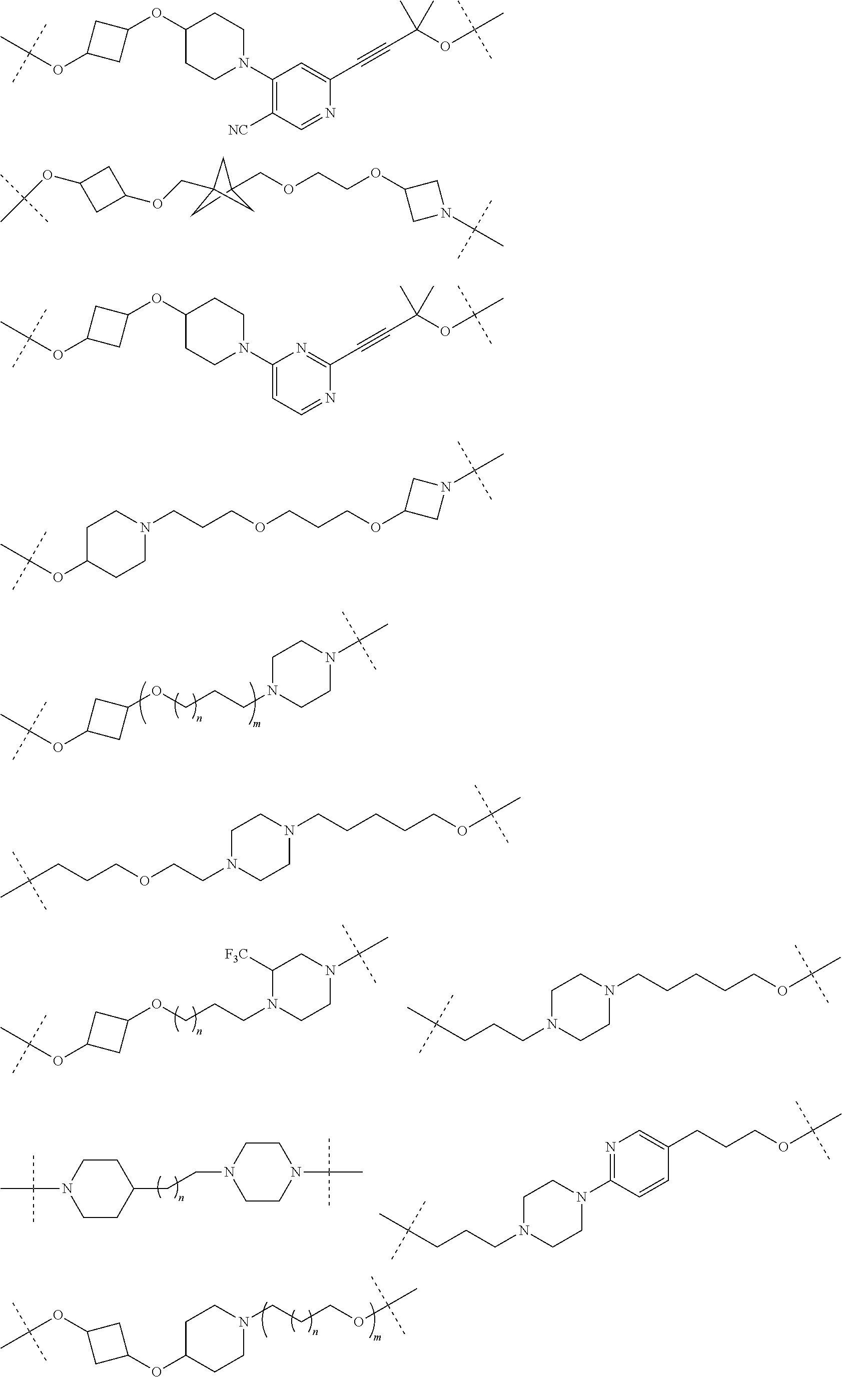 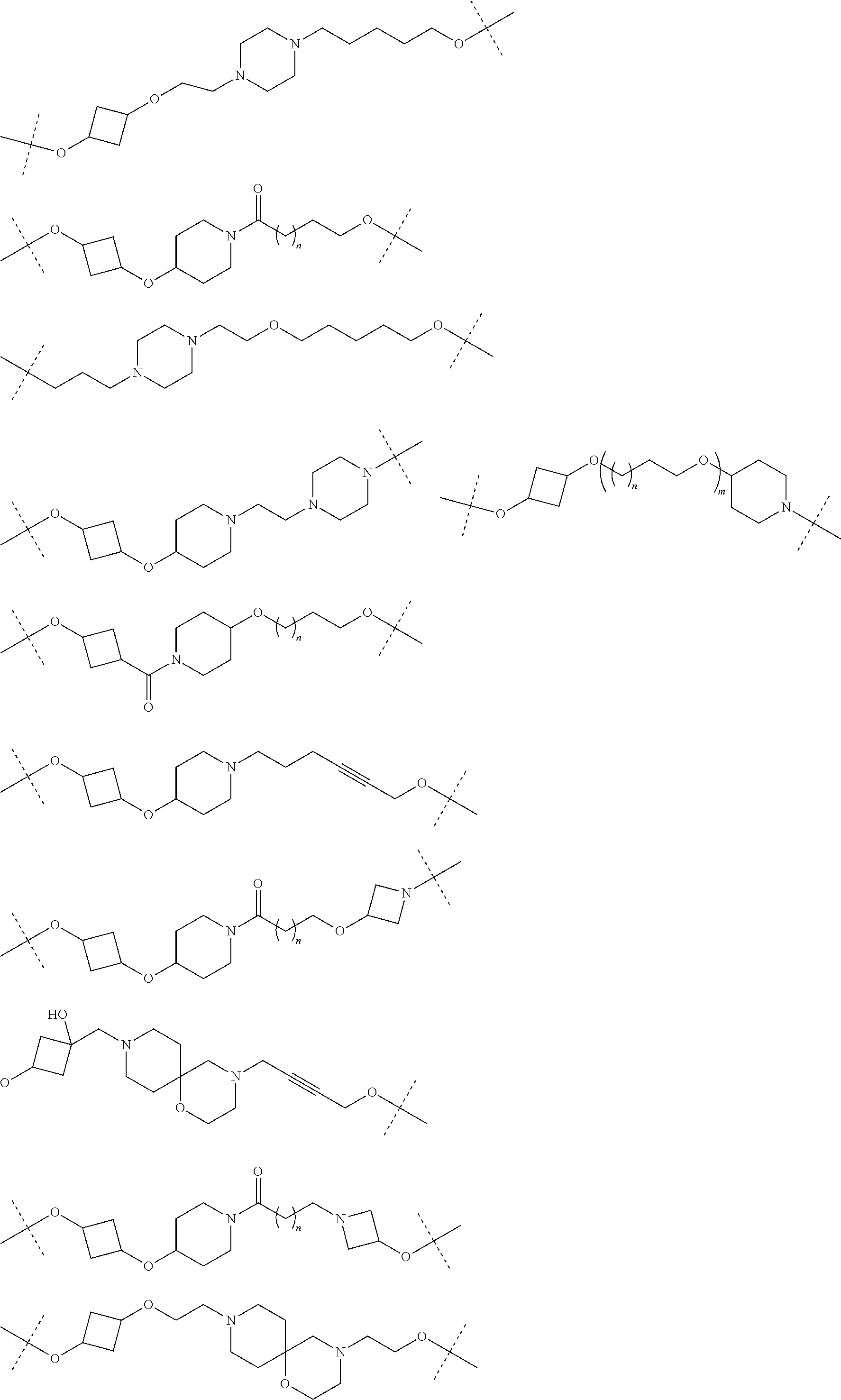 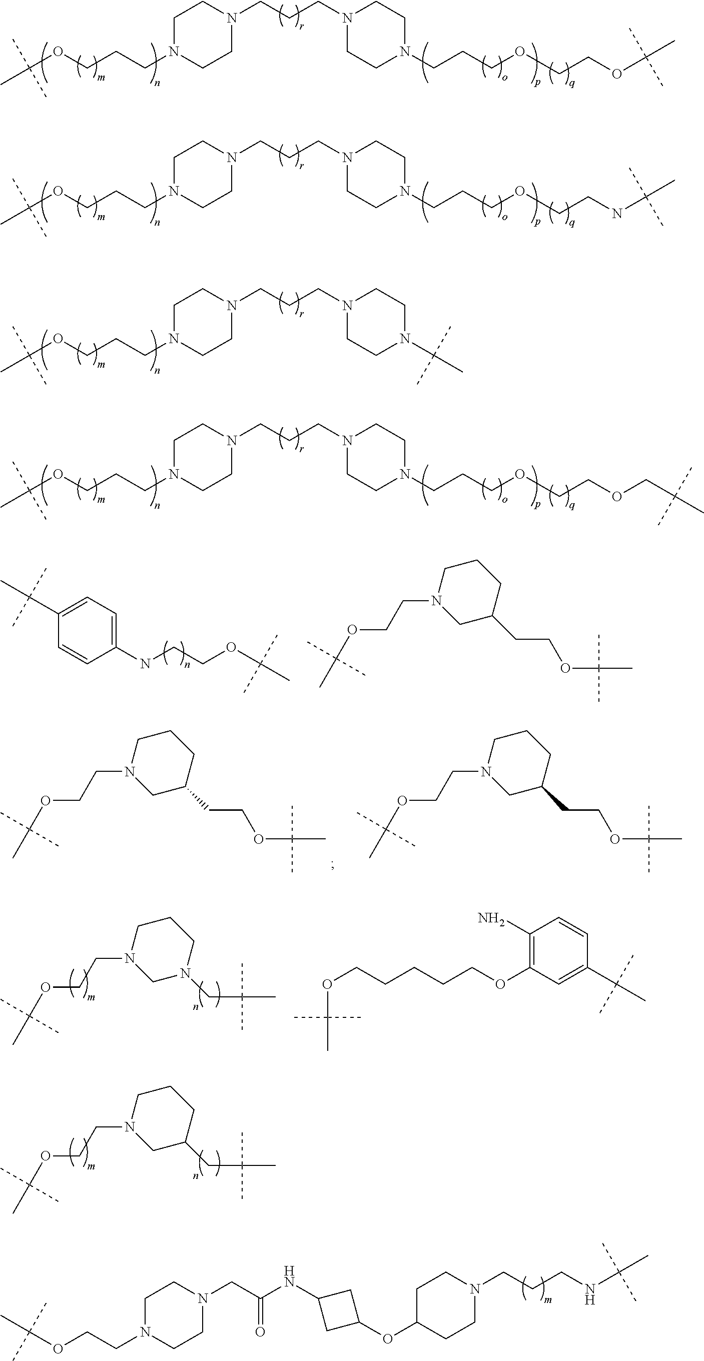 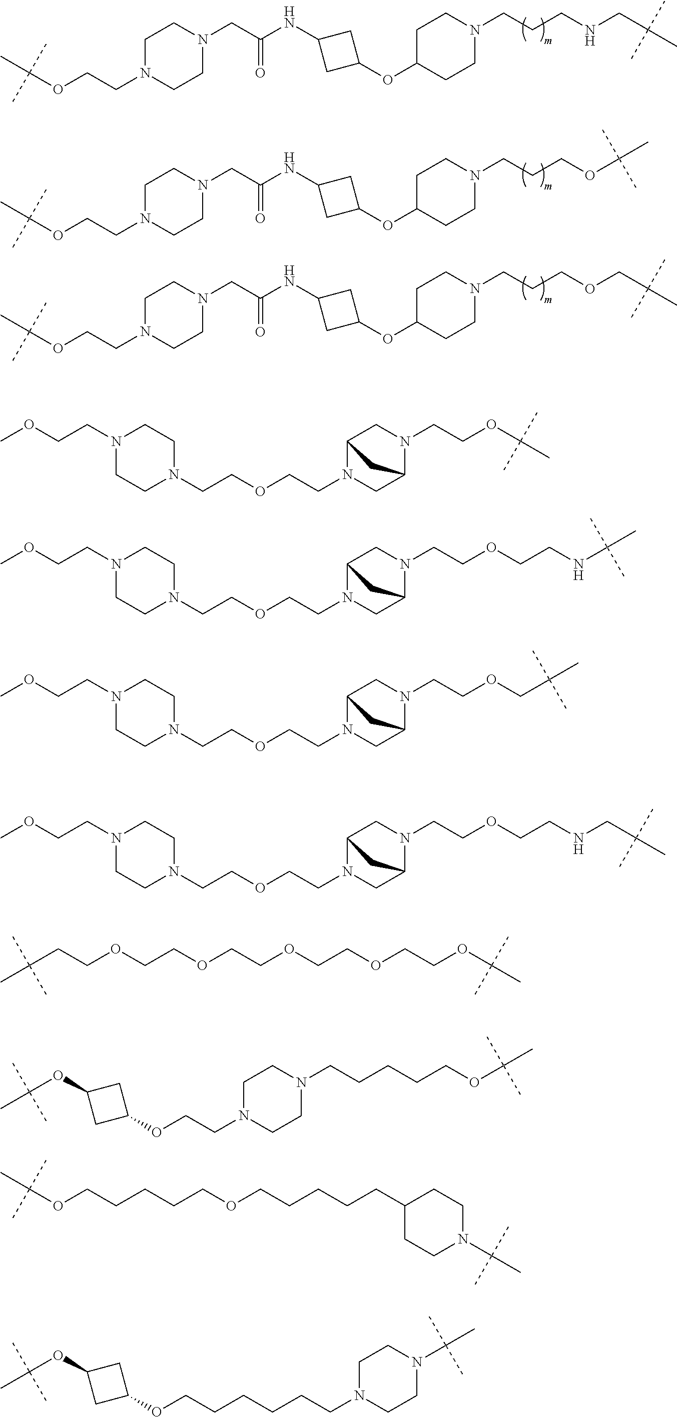 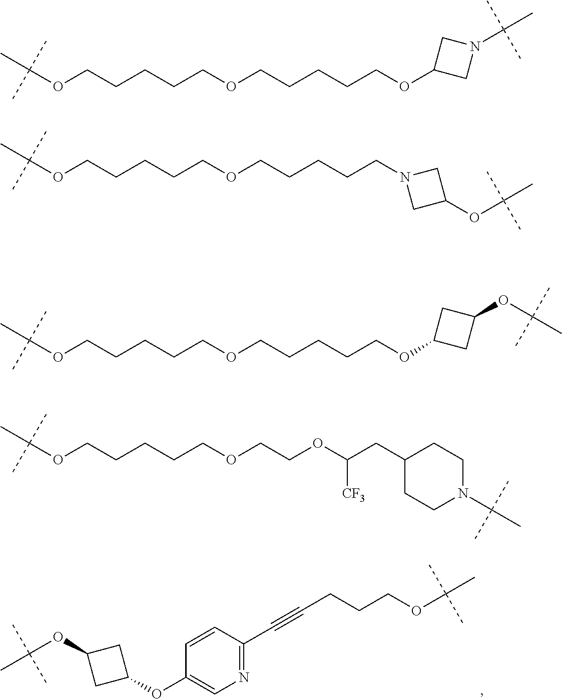
wherein each m, n, o, p, q, r, and s is independently 0, 1, 2, 3, 4, 5, 6, 7, 8, 9, 10, 11, 12, 13, 14, 15, 16, 17, 18, 19, or 20.

19. The compound according to claim 15, wherein the unit A^{L} of linker (L) is selected from:

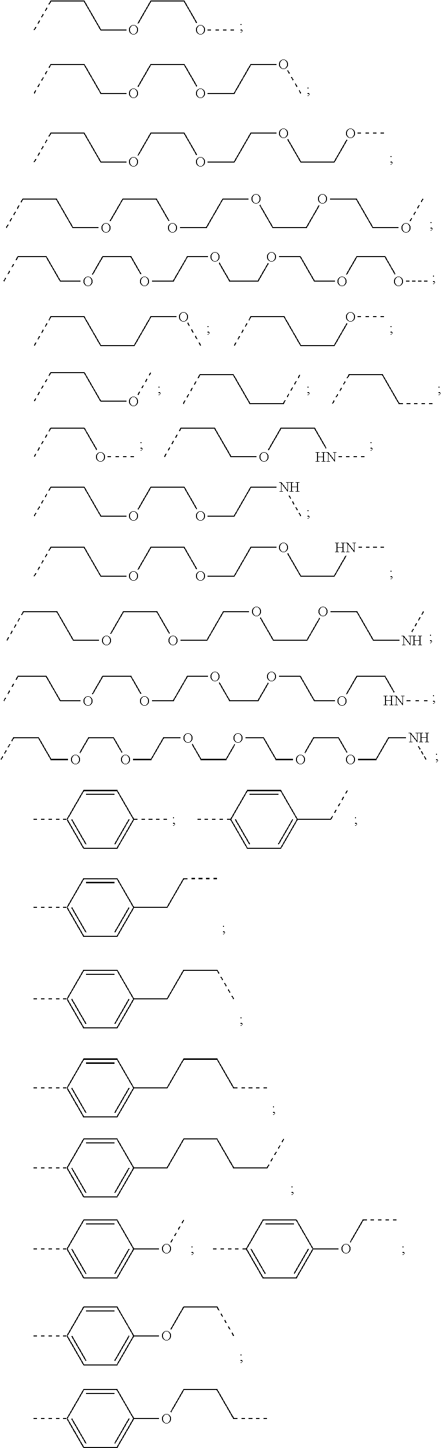

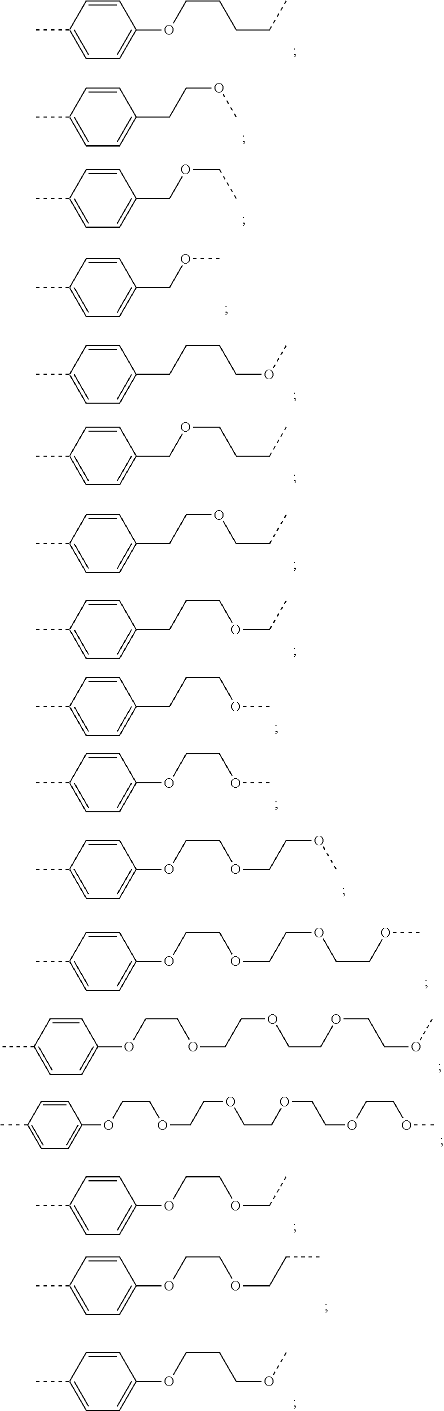

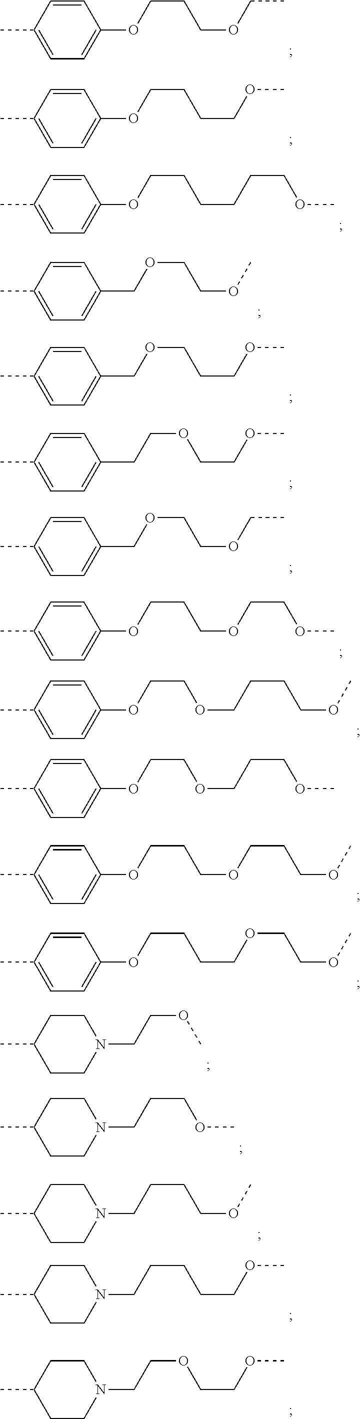

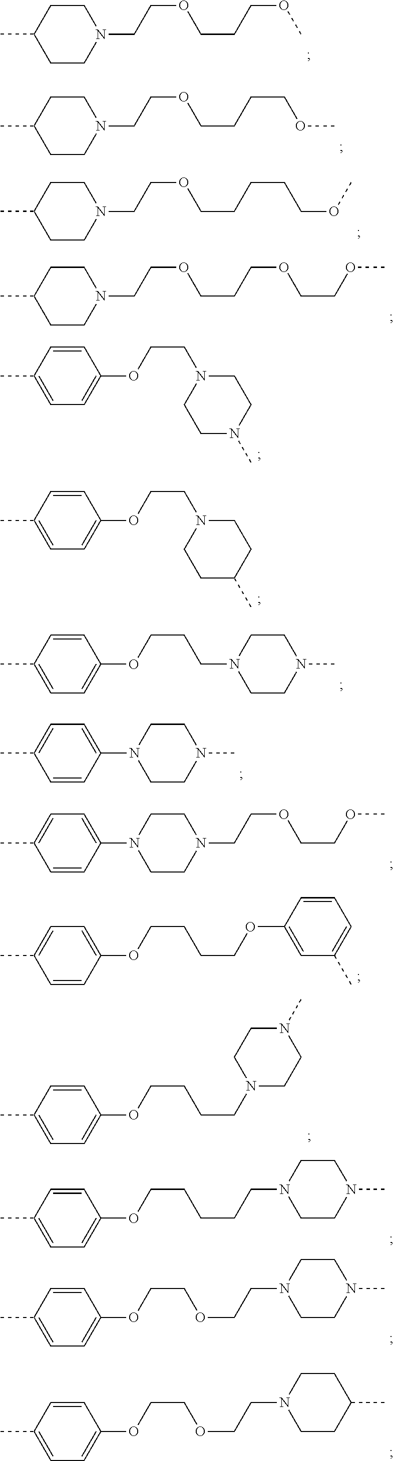

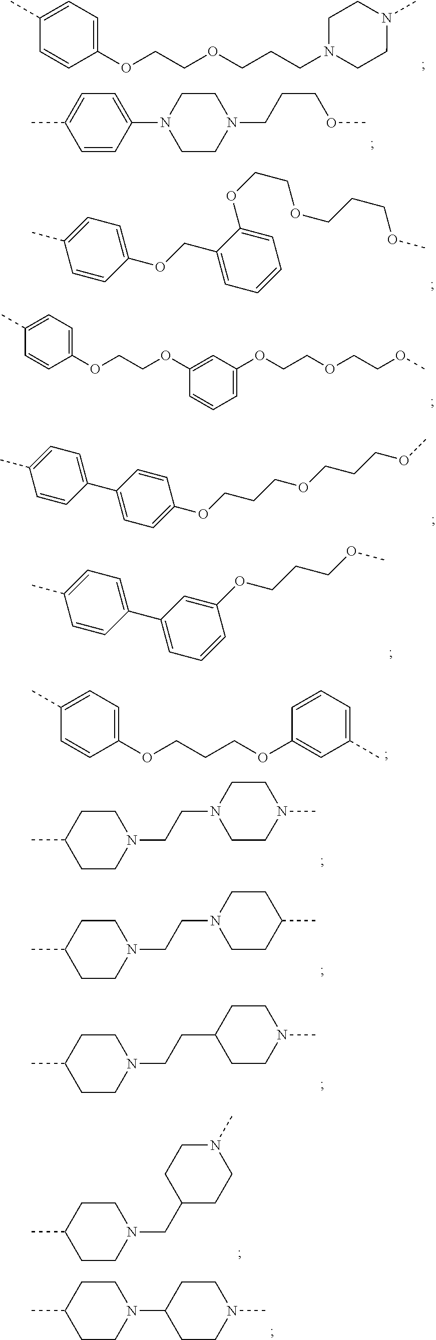

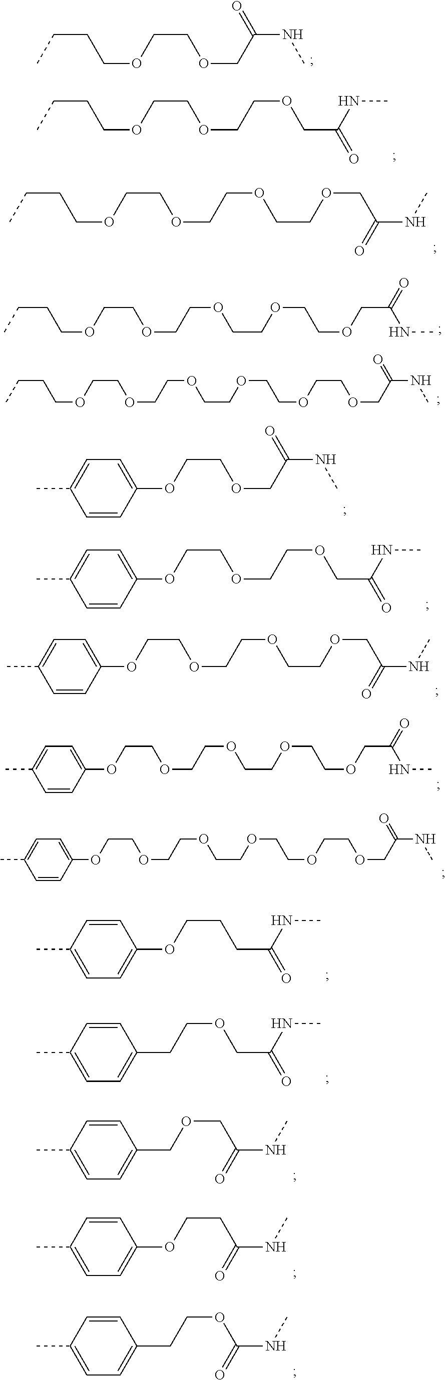

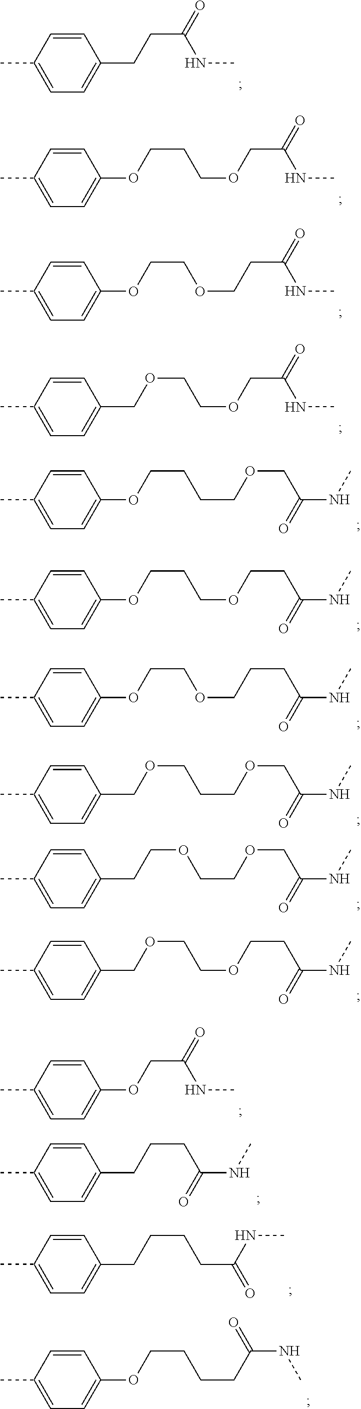

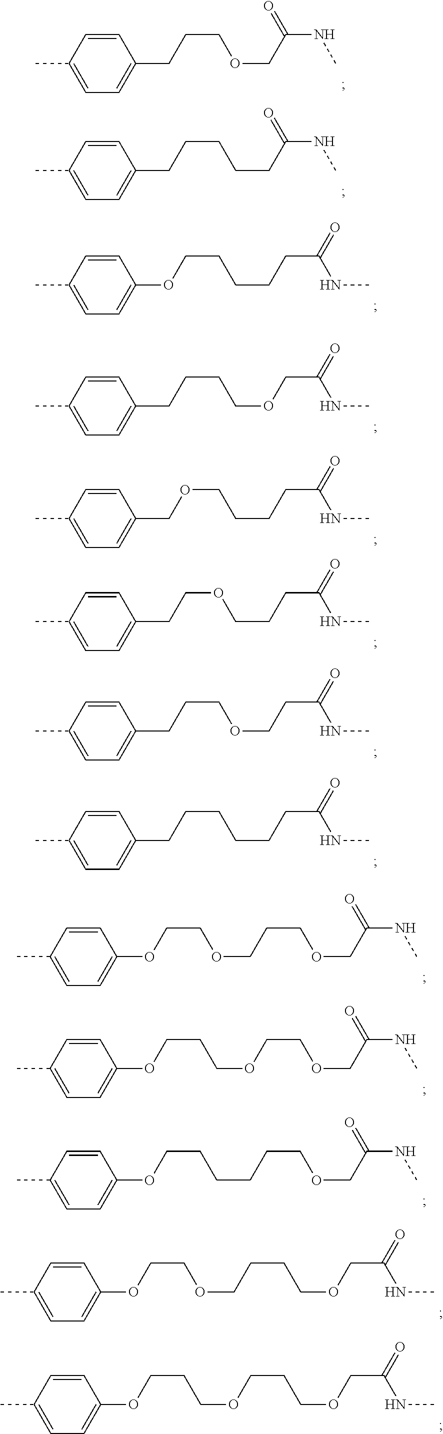

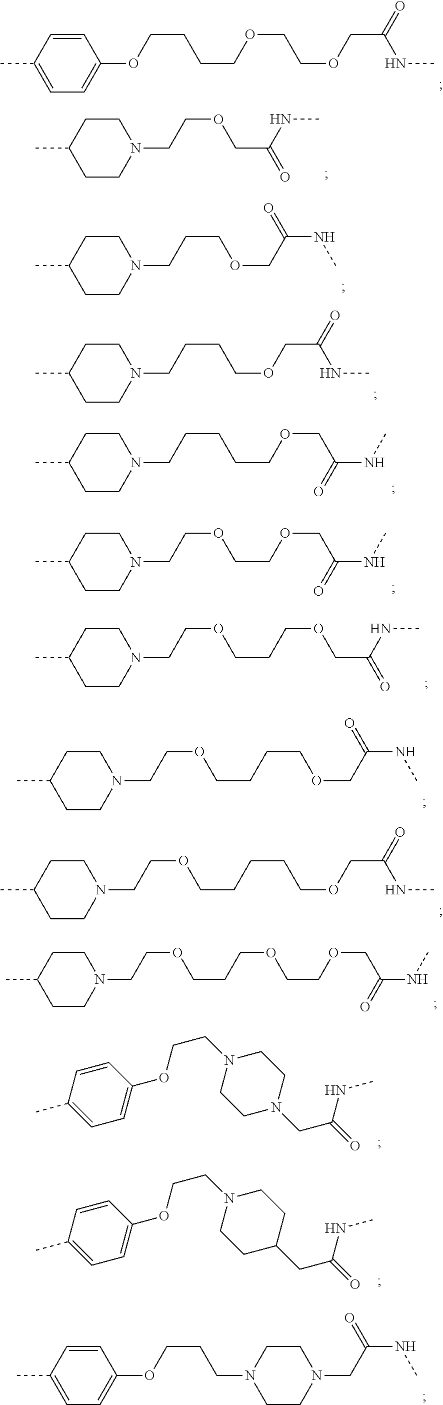

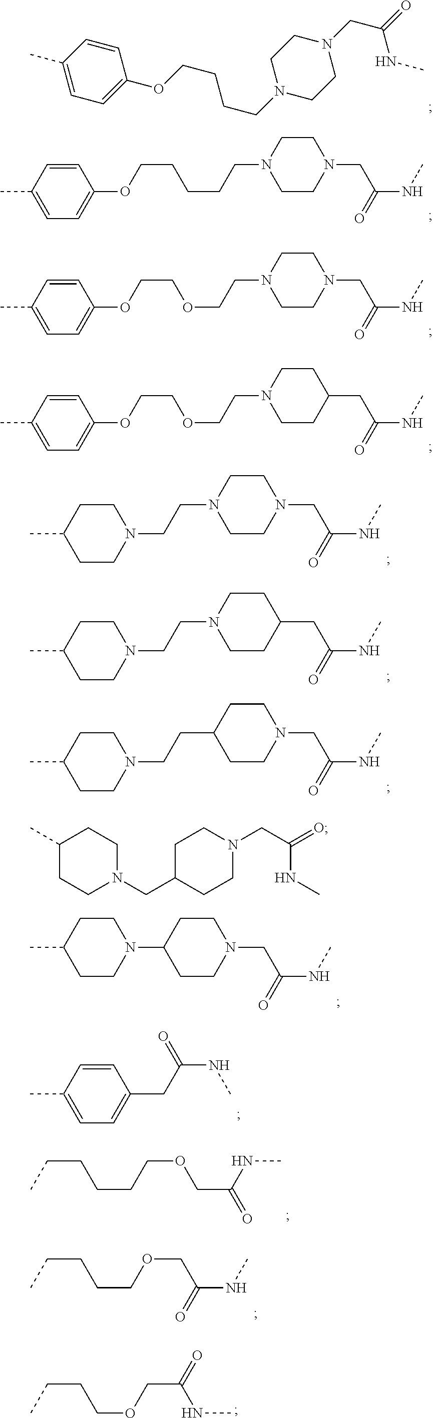

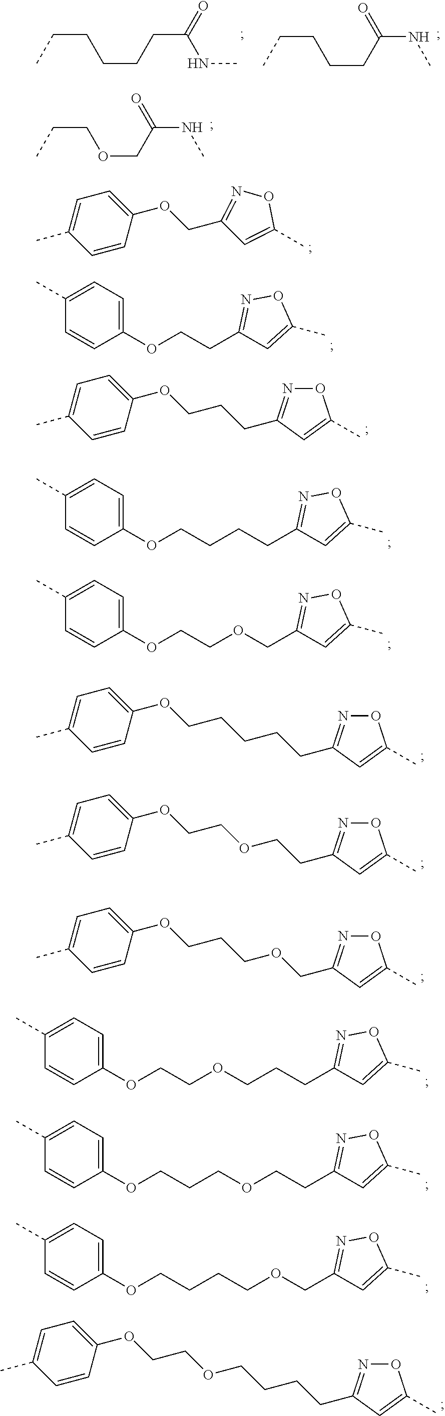

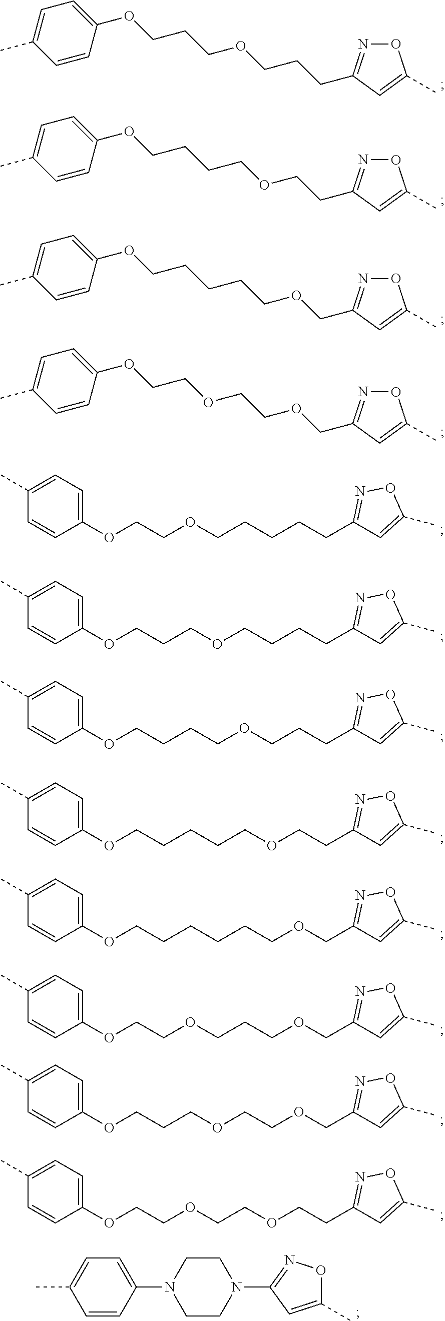

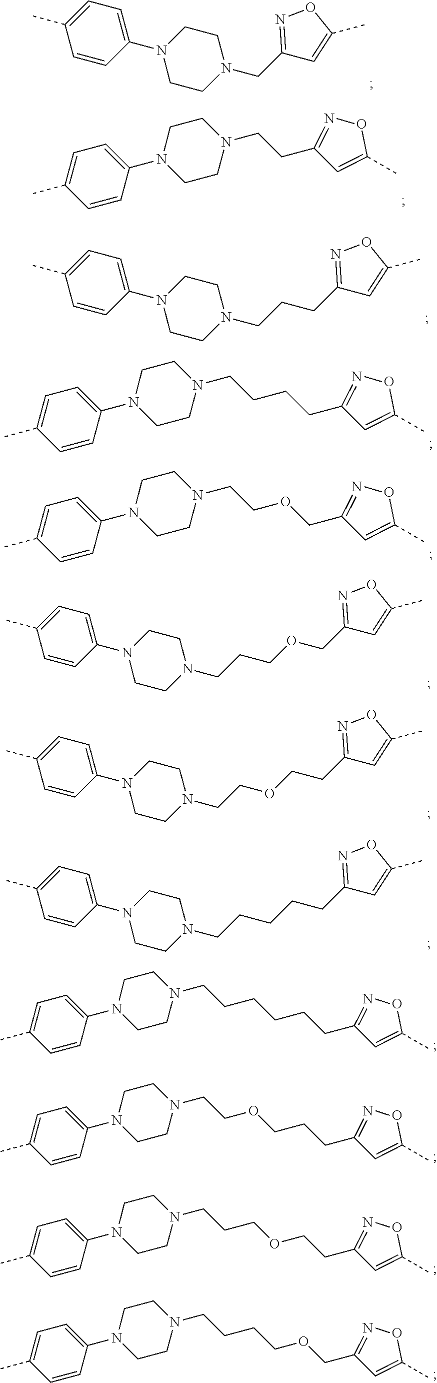

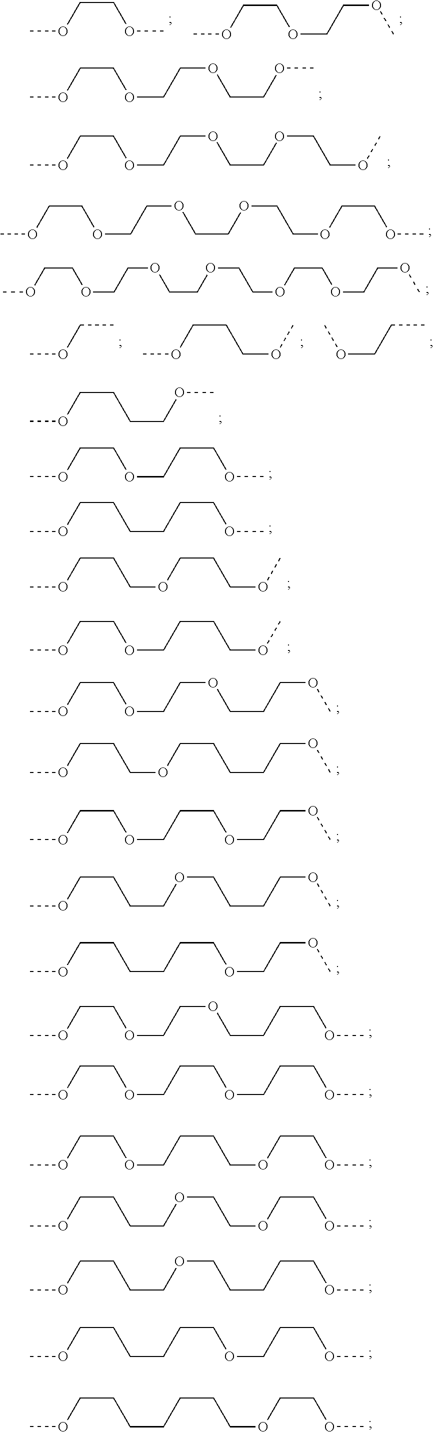

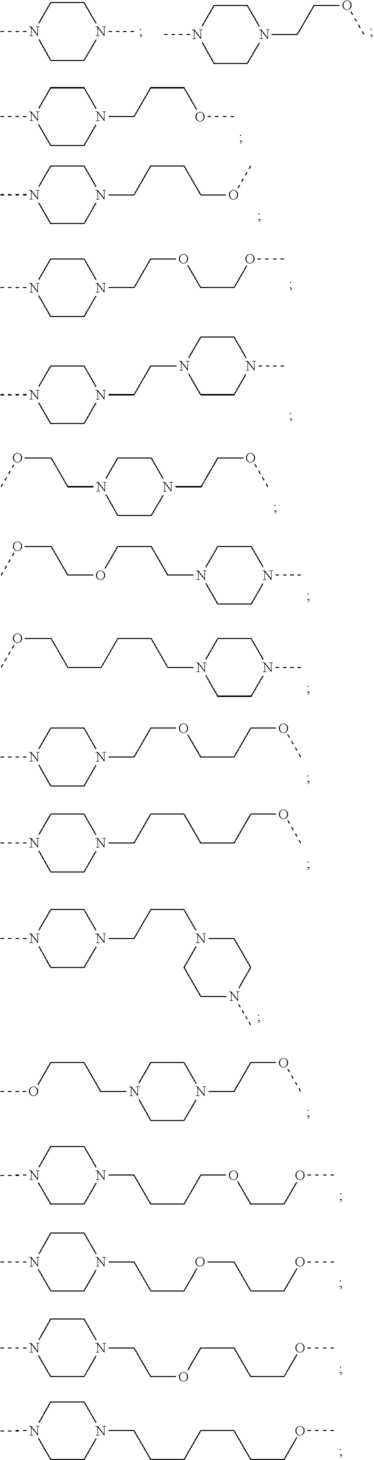

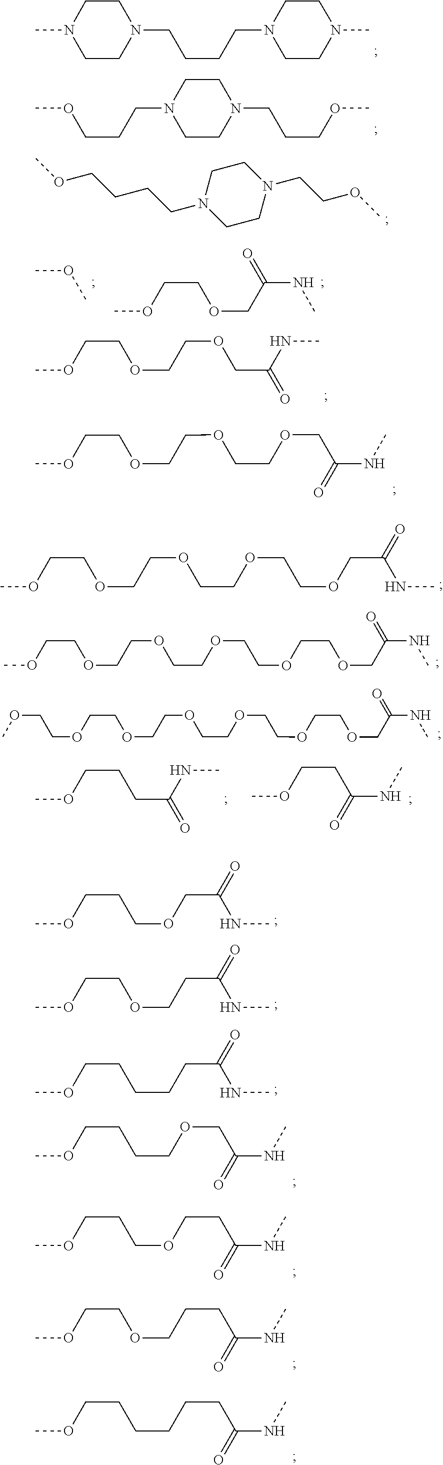

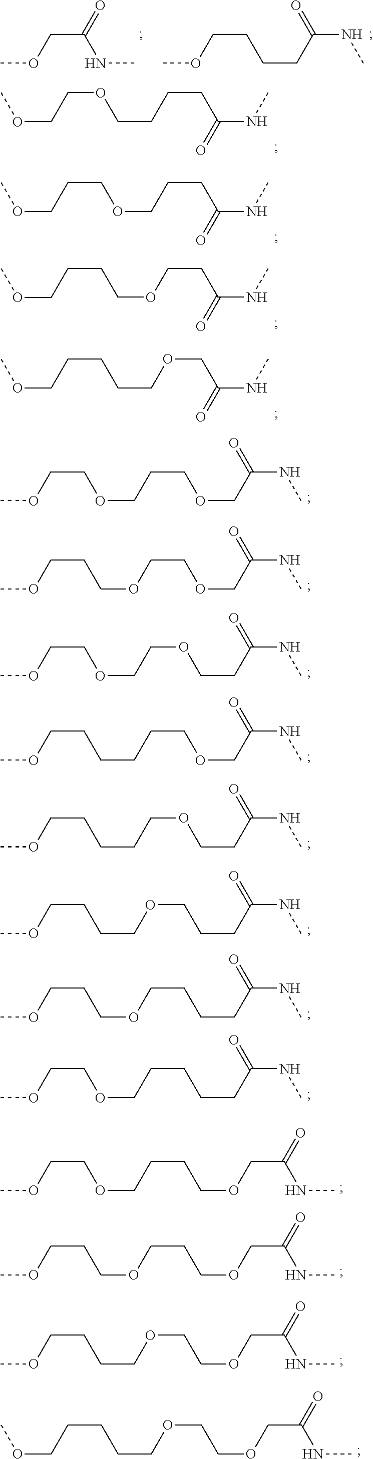

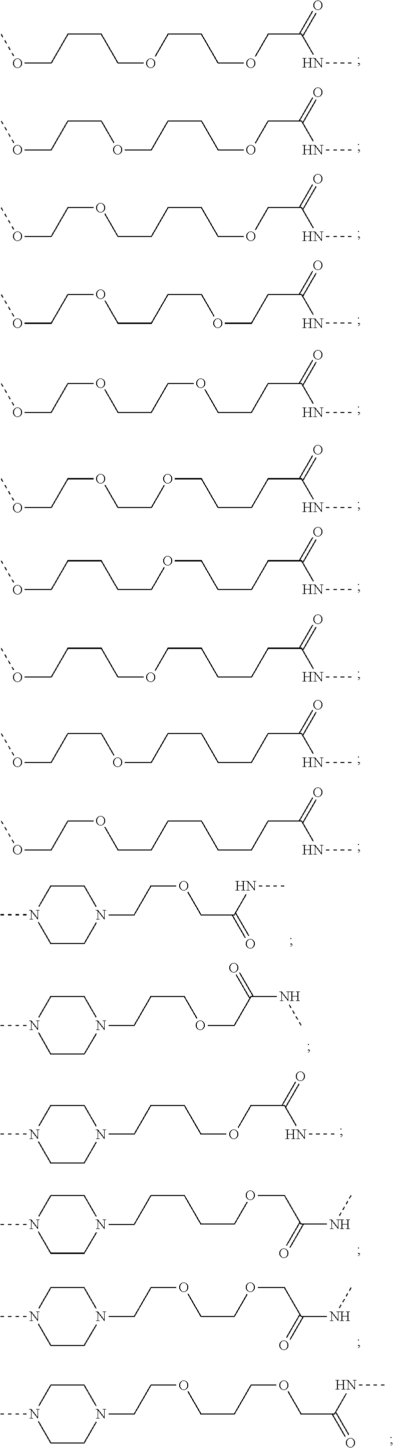

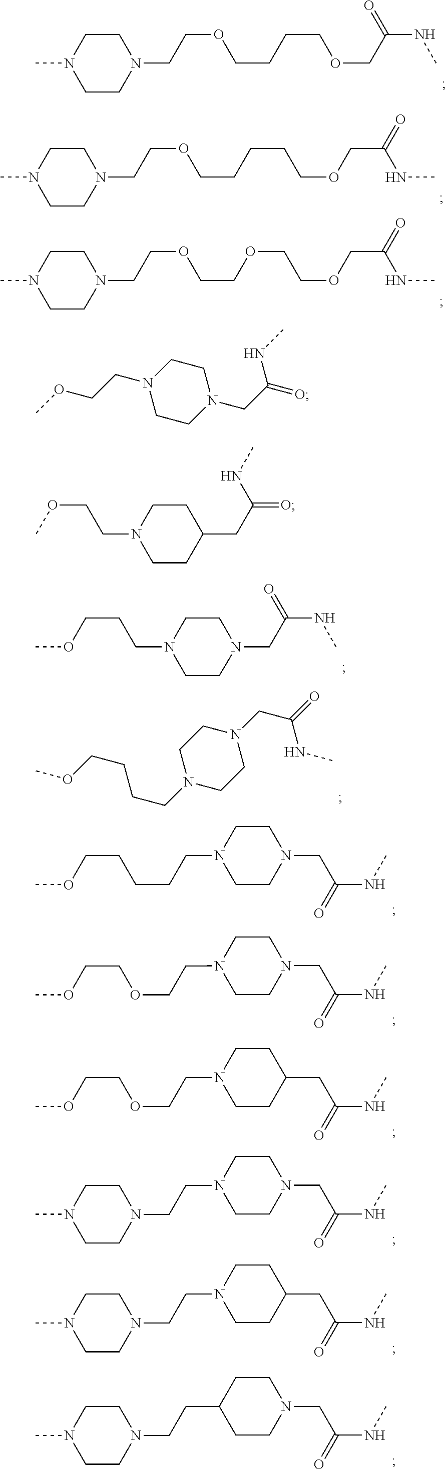

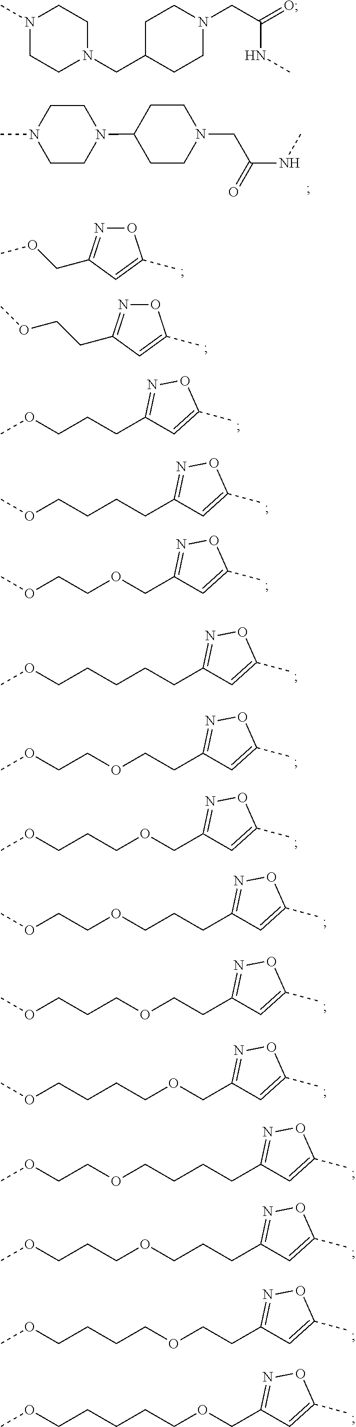

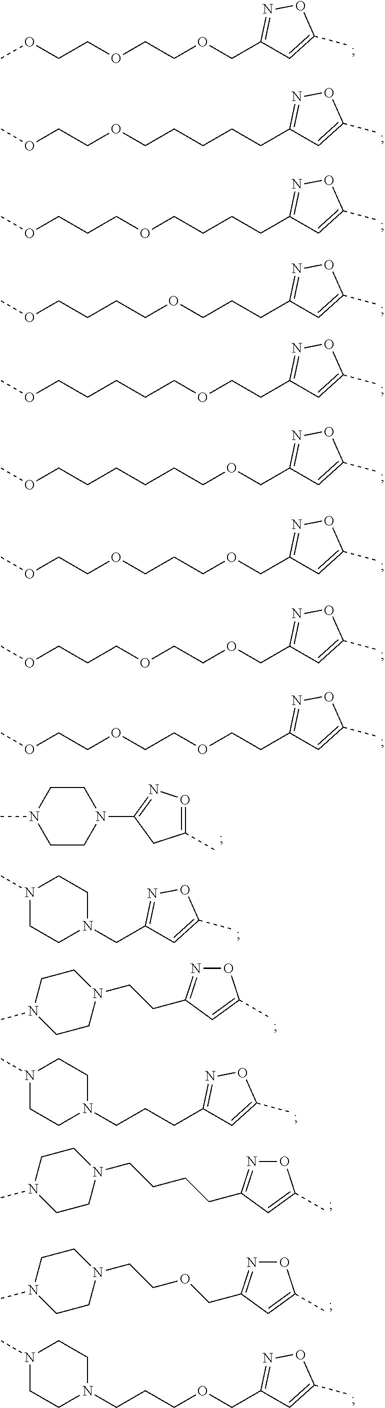

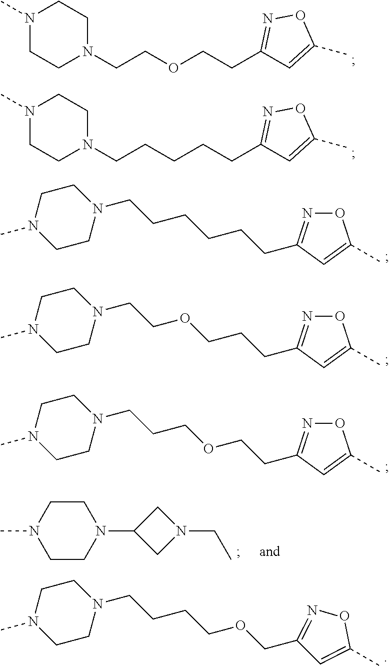

20. The compound according to claim 15, wherein the linker (L) is a polyethylenoxy group optionally substituted with aryl or phenyl comprising from 1 to 10 ethylene glycol units.

21. The compound according to claim 1, wherein the linker (L) comprises the following chemical structure: 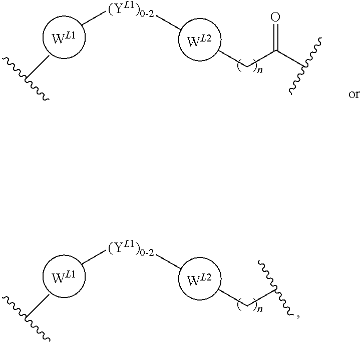
wherein:
W^{L1} and W¹² are each independently absent, a 4-8 membered ring with 0-4 heteroatoms, optionally substituted with RQ, each RQ is independently a H, halo, OH, CN, CF3, optionally substituted linear or branched C1-C6 alkyl, optionally substituted linear or branched C1-C6 alkoxy, or 2 RQ groups taken together with the atom they are attached to, form a 4-8 membered ring system containing 0-4 heteroatoms;
Y^{L1} is each independently a bond, optionally substituted linear or branched C1-C6 alkyl and optionally one or more C atoms are replaced with O; or optionally substituted linear or branched C1-C6 alkoxy;
n is 0-10; and 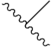
indicates the attachment point to the PTM or ULM moieties.

22. The compound according to claim 1, wherein the linker (L) comprises the following chemical structure: 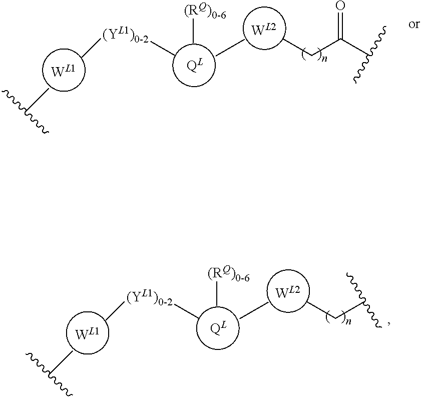
wherein:
W^{L1} and W^{L2} are each independently absent, aryl, heteroaryl, cyclic, heterocyclic, C₁₋₆ alkyl and optionally one or more C atoms are replaced with O, C₁₋₆ alkene and optionally one or more C atoms are replaced with O, C₁₋₆ alkyne and optionally one or more C atoms are replaced with O, bicyclic, biaryl, biheteroaryl, or biheterocyclic, each optionally substituted with R^{Q}, each R^{Q} is independently a H, halo, OH, CN, CF₃, hydroxyl, nitro, C≡CH, C₂₋₆ alkenyl, C₂₋₆ alkynyl, optionally substituted linear or branched C₁-C₆ alkyl, optionally substituted linear or branched C₁-C₆ alkoxy, OC₁₋₃alkyl, OH, NH₂, NR^{Y1}R^{Y2}, CN, or 2R^{Q} groups taken together with the atom they are attached to, form a 4-8 membered ring system containing 0-4 heteroatoms;
Y^{L1} is each independently a bond, NR^{YL1}, O, S, NR^{YL2}, CR^{YL1}R^{YL2}, C═O, C═S, SO, SO₂, optionally substituted linear or branched C₁-C₆ alkyl and optionally one or more C atoms are replaced with O; optionally substituted linear or branched C₁-C₆ alkoxy;
Q^{L} is a 3-6 membered alicyclic or aromatic ring with 0-4 heteroatoms, optionally bridged, optionally substituted with 0-6 R^{Q}, each R^{Q} is independently H, optionally substituted linear or branched C₁₋₆ alkyl, or 2 R^{Q} groups taken together with the atom they are attached to, form a 3-8 membered ring system containing 0-2 heteroatoms;
R^{YL1}, R^{YL2} are each independently H, OH, optionally substituted linear or branched C₁₋₆ alkyl, or R¹, R² together with the atom they are attached to, form a 3-8 membered ring system containing 0-2 heteroatoms;
n is 0-10; and 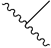
indicates the attachment point to the PTM or ULM moieties.

23. The compounds according to claim 15, wherein the linker (L) or the unit A^{L} of linker (L) is selected from the group consisting of:

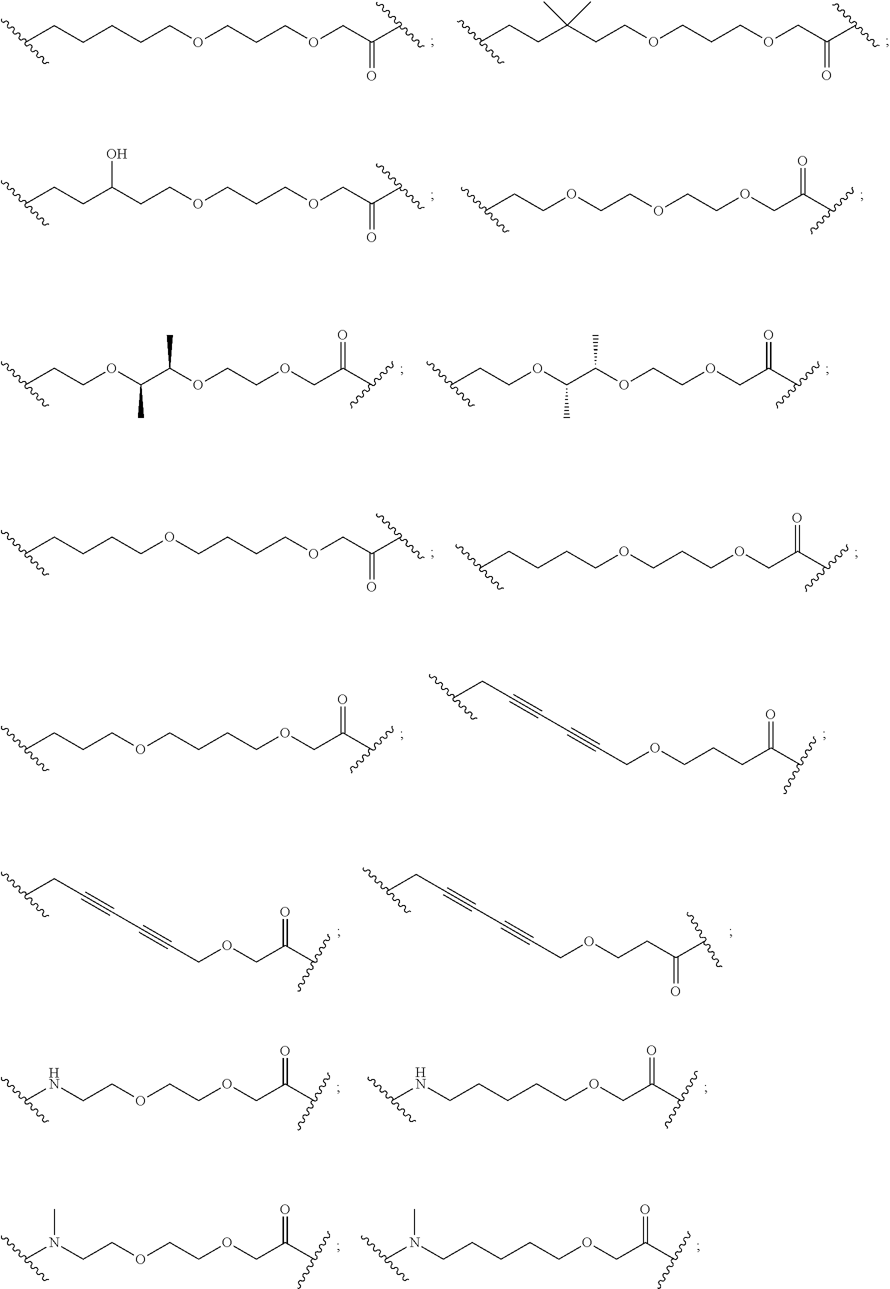

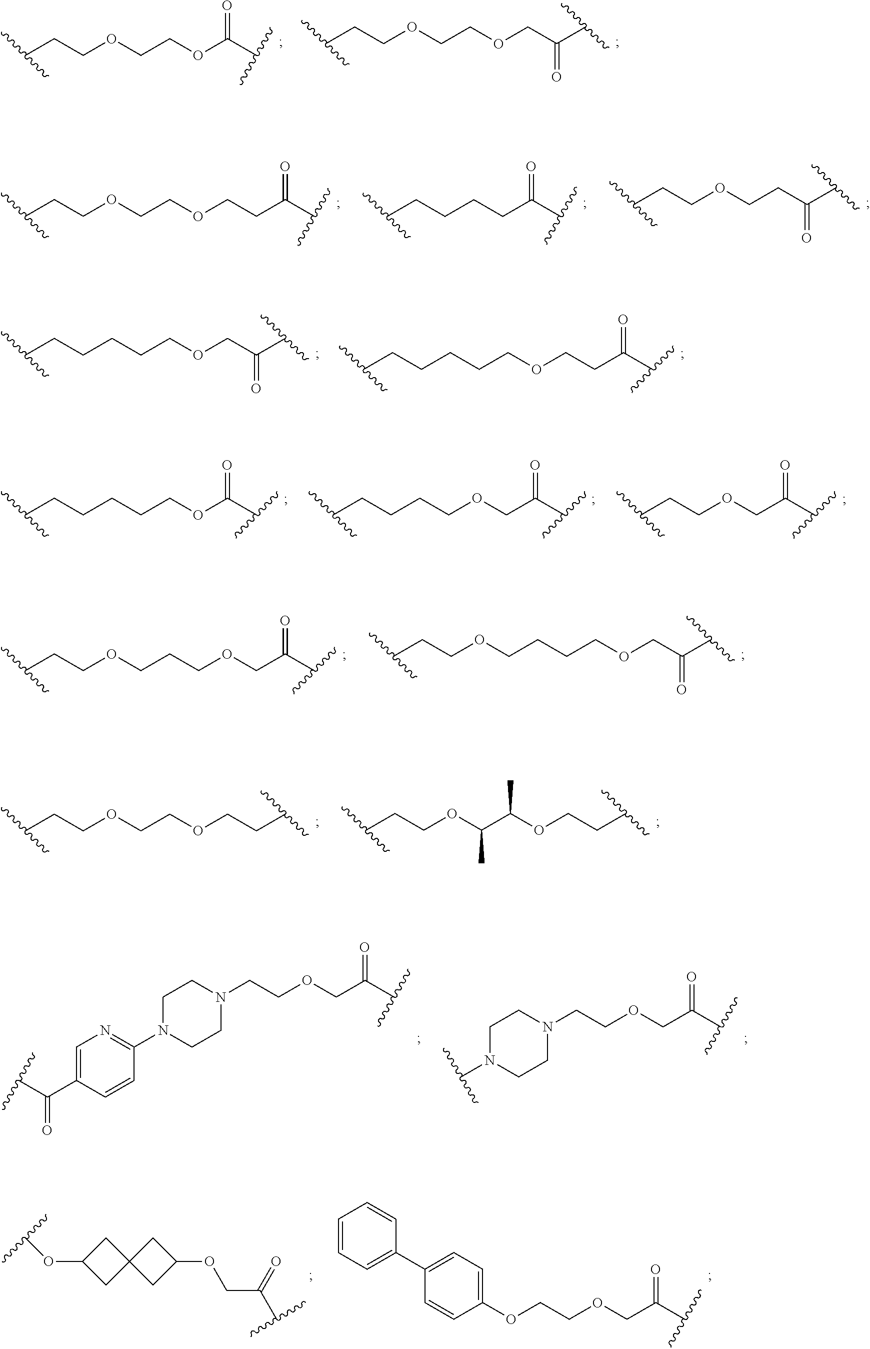

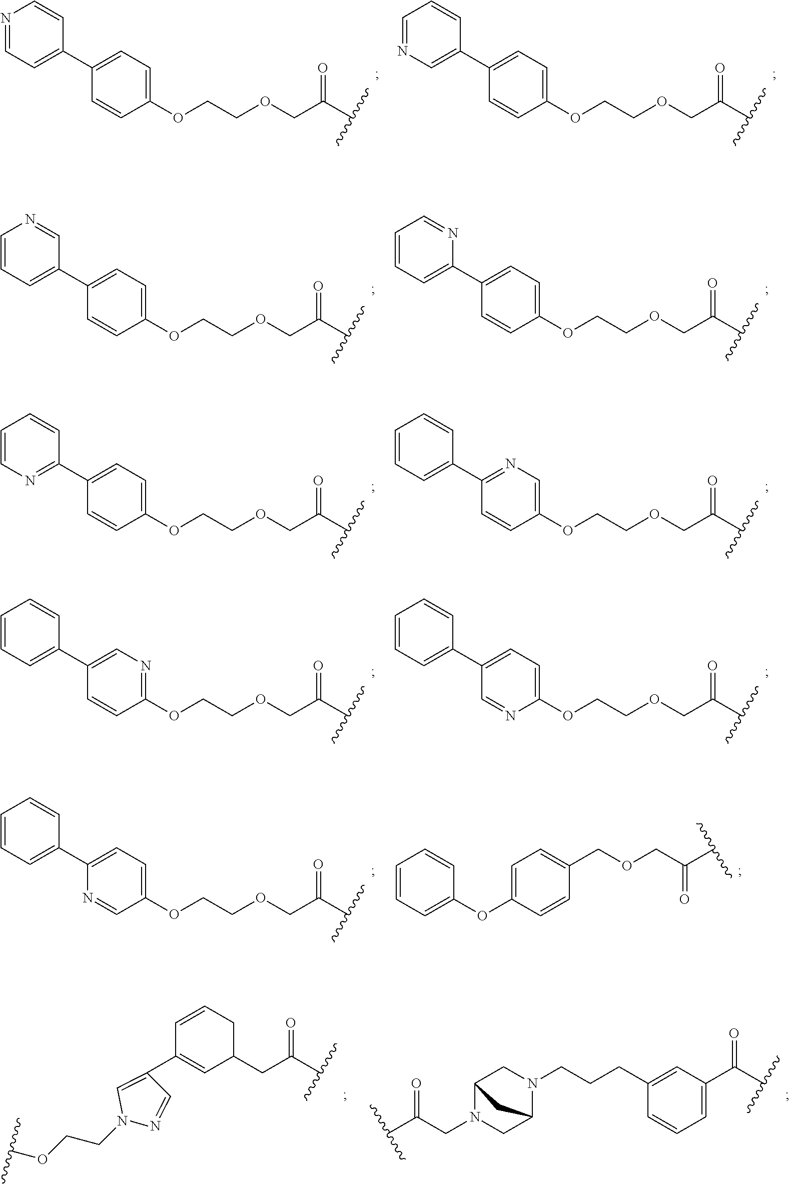

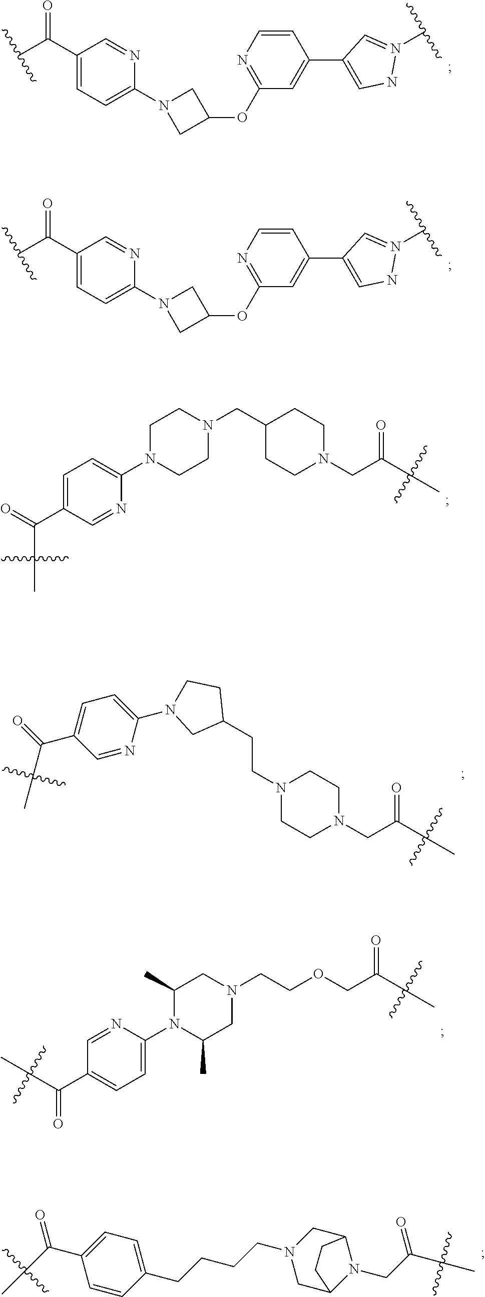

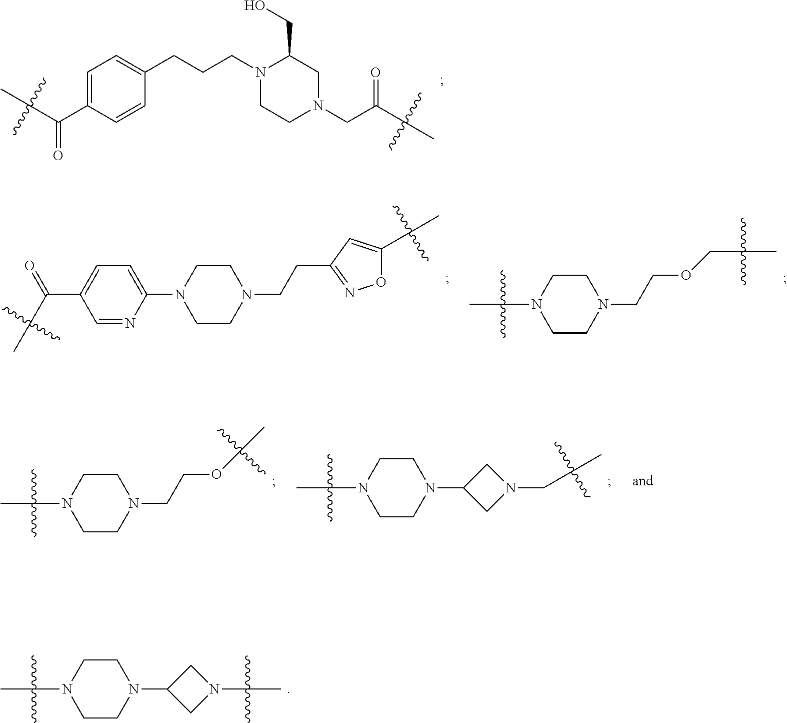 [SMILES: *C(=O)CN1CCN(CCCc2ccc(C(*)=O)cc2)[C@@H](CO)C1 ; *C(=O)c1ccc(N2CCN(CCc3cc(*)on3)CC2)nc1 ; *COCCN1CCN(*)CC1 ; *OCCN1CCN(*)CC1 ; *CN1CC(N2CCN(*)CC2)C1 ; *N1CCN(C2CN(*)C2)CC1]

24. The compound according claim 1, wherein the compound comprises multiple ULMs, multiple PTMs, multiple linkers or any combinations thereof.

25. The bifunctional compound of claim 1, wherein the compound is selected from the group consisting of:

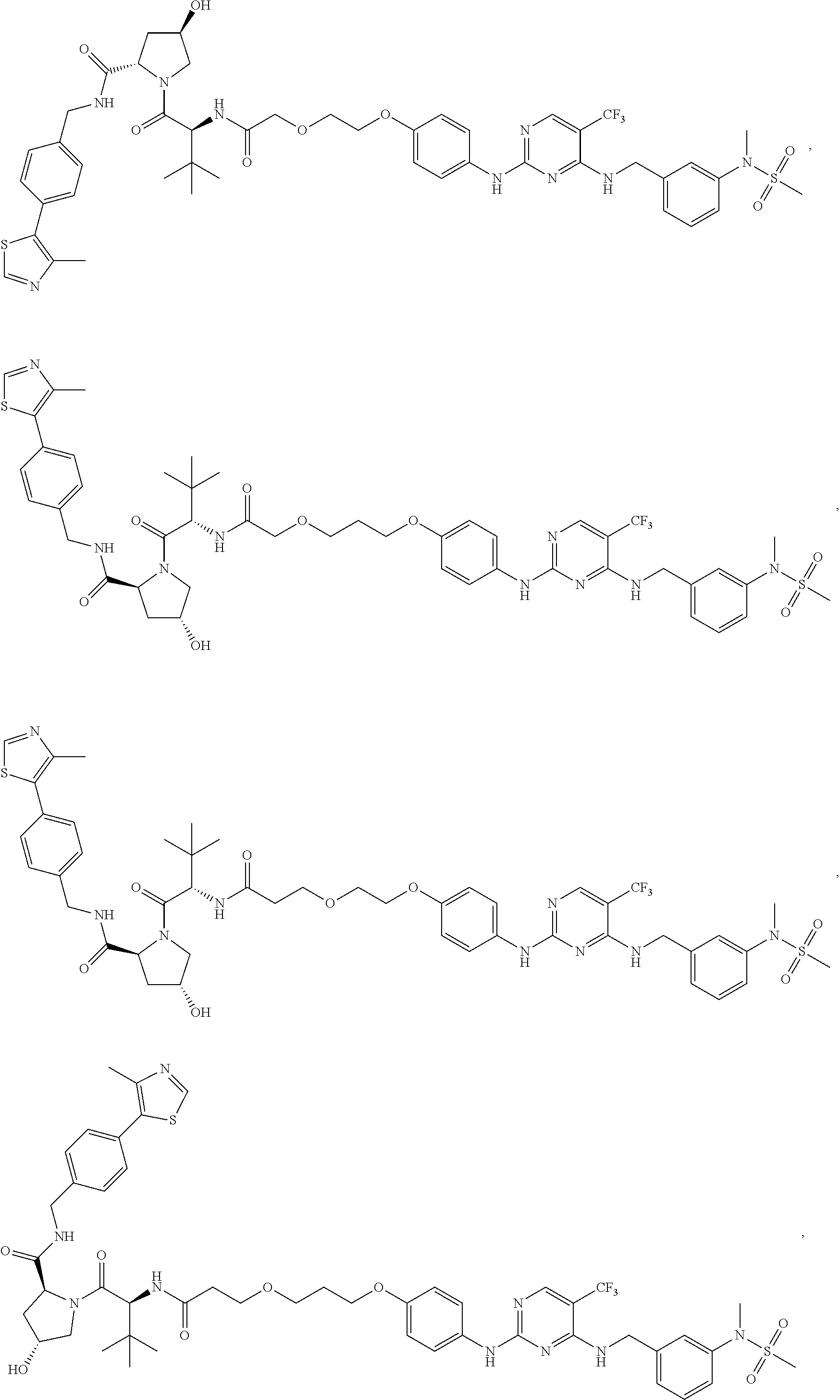

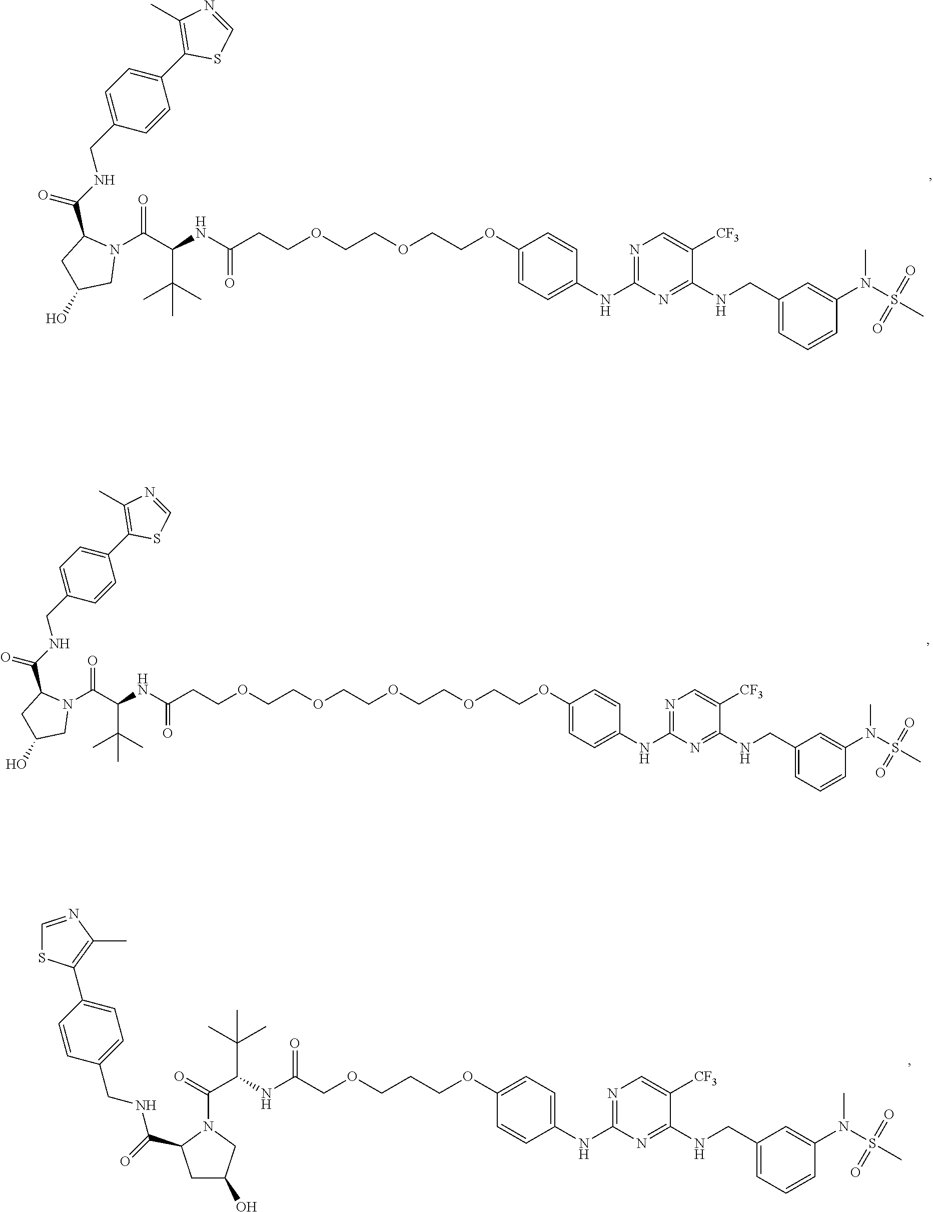 [SMILES: Cc1ncsc1-c1ccc(CNC(=O)[C@@H]2C[C@@H](O)CN2C(=O)[C@@H](NC(=O)CCOCCOCCOc2ccc(Nc3ncc(C(F)(F)F)c(NCc4cccc(N(C)S(C)(=O)=O)c4)n3)cc2)C(C)(C)C)cc1 ; Cc1ncsc1-c1ccc(CNC(=O)[C@@H]2C[C@@H](O)CN2C(=O)[C@@H](NC(=O)CCOCCOCCOCCOCCOc2ccc(Nc3ncc(C(F)(F)F)c(NCc4cccc(N(C)S(C)(=O)=O)c4)n3)cc2)C(C)(C)C)cc1 ; Cc1ncsc1-c1ccc(CNC(=O)[C@@H]2C[C@H](O)CN2C(=O)[C@@H](NC(=O)COCCCOc2ccc(Nc3ncc(C(F)(F)F)c(NCc4cccc(N(C)S(C)(=O)=O)c4)n3)cc2)C(C)(C)C)cc1]

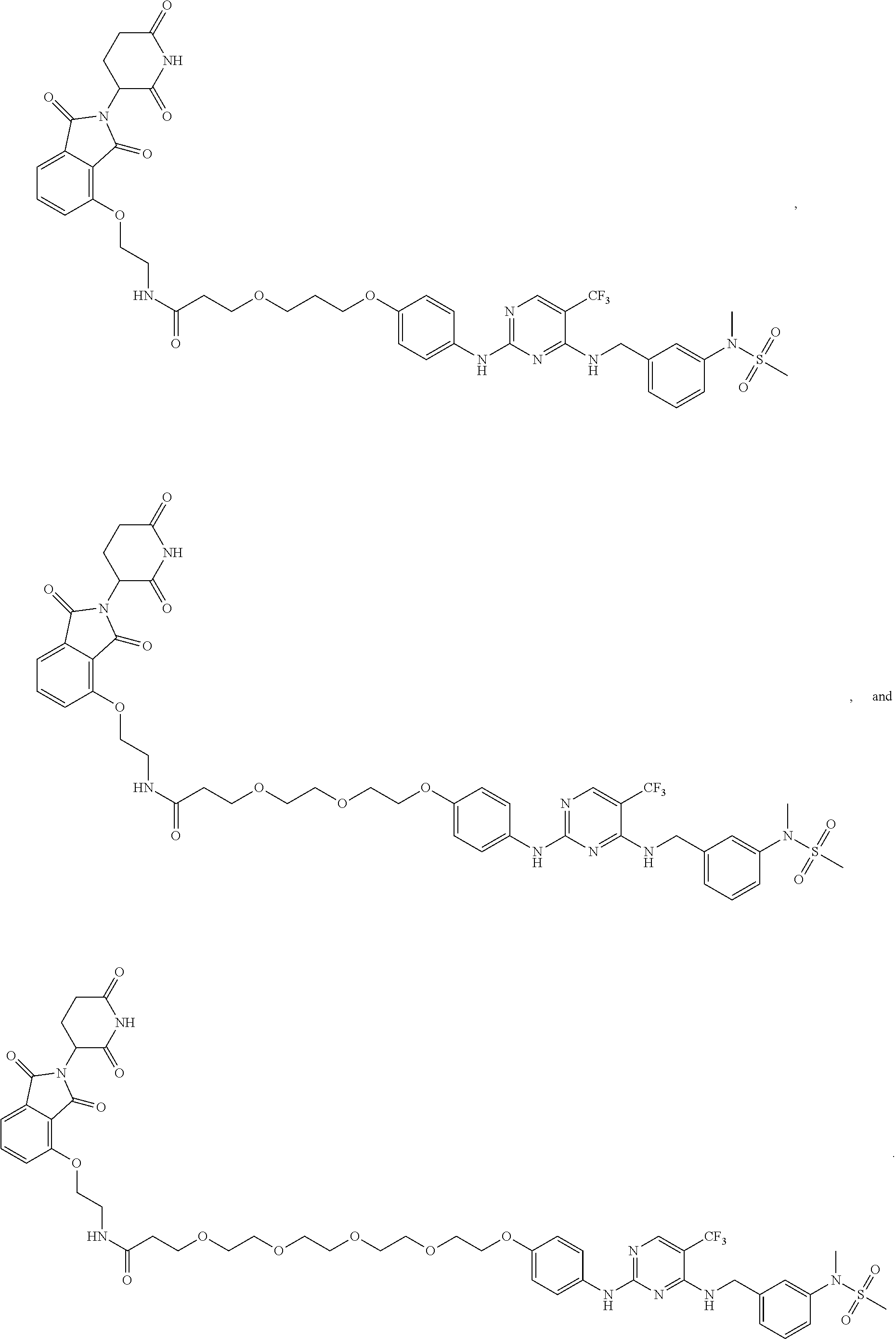 [SMILES: CN(c1cccc(CNc2nc(Nc3ccc(OCCCOCCC(=O)NCCOc4cccc5c4C(=O)N(C4CCC(=O)NC4=O)C5=O)cc3)ncc2C(F)(F)F)c1)S(C)(=O)=O ; CN(c1cccc(CNc2nc(Nc3ccc(OCCOCCOCCC(=O)NCCOc4cccc5c4C(=O)N(C4CCC(=O)NC4=O)C5=O)cc3)ncc2C(F)(F)F)c1)S(C)(=O)=O ; CN(c1cccc(CNc2nc(Nc3ccc(OCCOCCOCCOCCOCCC(=O)NCCOc4cccc5c4C(=O)N(C4CCC(=O)NC4=O)C5=O)cc3)ncc2C(F)(F)F)c1)S(C)(=O)=O]

26. A composition comprising an effective amount of a bifunctional compound of claim 1, and a pharmaceutically acceptable carrier.

27. The composition of claim 26, wherein the composition further comprises at least one of additional bioactive agent or another bifunctional compound of claim 1.

28. The composition of claim 27, wherein the additional bioactive agent is an anti-cancer agent.

29. A method of treating a disease or disorder in a subject, the method comprising administering a composition comprising a pharmaceutically acceptable carrier and an effective amount of at least one compound of claim 1 to a subject in need thereof, wherein the compound is effective in treating or ameliorating at least one symptom of the disease or disorder.

30. The method of claim 29, wherein the disease or disorder is associated with at least one of one of accumulation, aggregation, overactivation, or combinations thereof, of FAKs.

31. The method of claim 29, wherein the disease or disorder is cancer that is associated with the accumulation, aggregation, and/or overactivation of FAKs.

32. The method of claim 29, wherein the disease or disorder is a solid tumor, carcinoma, adenocarcinoma, cystadenocarcinoma, endometriod carcinoma, mesothelioma, sarcoma, breast cancer, ovarian cancer, lung cancer, head and neck cancer, colorectal cancer, bladder cancer, uterine cancer, prostate cancer, squamous cell carcinoma, leukemia, glioblastoma and renal cancer.

33. The method of claim 29, wherein the disease or disorder is ovarian cancer or breast cancer.
